(19) United States
(12) Patent Application Publication
(10) Pub. No.: US 2023/0052369 A1
(43) Pub. Date: Feb. 16, 2023

(54) ANTIBODY CONSTRUCTS BINDING 4-1BB AND TUMOR-ASSOCIATED ANTIGENS AND USES THEREOF

(71) Applicant: Zymeworks Inc., Vancouver (CA)

(72) Inventors: Daniel T. PATTON, Vancouver (CA); David M. MILLS, Seattle, WA (US); Thomas SPRETER VON KREUDENSTEIN, Vancouver (CA); Gesa VOLKERS, Vancouver (CA); Dunja UROSEV, Vancouver (CA); Lee FREIBURGER, Vancouver (CA); Zhuang DUAN, Vancouver (CA); Elizabeth HALVORSEN, Vancouver (CA); Harsh PRATAP, Vancouver (CA); Brandon CLAVETTE, Vancouver (CA); Anna VON ROSSUM, Vancouver (CA); Duncan BROWMAN, Vancouver (CA); Peter Wing Yiu CHAN, Vancouver (CA); Danny CHUI, Vancouver (CA); Robert William GENE, Vancouver (CA); Sylwia JANCOWSKI, Vancouver (CA); Sukhbir Singh KANG, Vancouver (CA); Patricia ZWIERZCHOWSKI, Vancouver (CA)

(21) Appl. No.: 17/226,834

(22) Filed: Apr. 9, 2021

Related U.S. Application Data
(63) Continuation of application No. PCT/CA2019/051448, filed on Oct. 10, 2019.
(60) Provisional application No. 62/744,059, filed on Oct. 10, 2018.

(51) Int. Cl. C07K 16/28; C07K 16/46; C07K 16/30; C07K 16/32

(52) U.S. Cl. CPC C07K 16/2878; C07K 2317/94; C07K 16/28; C07K 16/30; C07K 16/32; C07K 2317/31; C07K 2317/35; C07K 2317/526; C07K 2317/622; C07K 2317/71; C07K 2317/64; C07K 2317/55; C07K 2317/24; C07K 2317/92; C07K 2317/33; C07K 2317/75; C07K 2317/624; C07K 16/468

(57) ABSTRACT

Described herein are antibody constructs comprising a 41-1BB binding domain and an antigen-binding domain that binds to a tumor-associated antigen (TAA), wherein the 4-1BB-binding domain and the TAA antigen-binding domain are linked directly or indirectly to a scaffold. The scaffold may be an Fc construct with modifications that reduce its ability to mediate effector function.

### CROSS REFERENCE TO RELATED APPLICATIONS

This application is a bypass continuation of International Application No. PCT/CA2019/051448, filed on Oct. 10, 2019, which claim the benefit of U.S. Provisional Application No. 62/744,059 filed on Oct. 10, 2018, which are all hereby incorporated in its entirety by reference.

### SEQUENCE LISTING

The instant application contains a Sequence Listing which will be submitted via EFS-Web and is hereby incorporated by reference in its entirety. Said ASCII copy, created on Sep. 3, 2021, is named ZWI-054C1_Sequence Listing.txt, and is 940,968 bytes in size.

### BACKGROUND

4-1BB is a member of the TNF receptor superfamily that is expressed on in several types of immune cells including, but not limited to, activated NK and NKT regulatory T cells, dendritic cells, B cells, and stimulated mast cells. Resting T cells do not express high levels of 4-1BB; it is upregulated after activation through the T cell receptor (TCR). Also known as CD137 or TNFRSF9, 4-1BB is expressed on non-immune cells as well, including populations of neural cells found in the brain (Bartkowiak and Curran (2015), Front. Oncol. 5:117). 4-1BB is a transmembrane receptor that is activated by binding to its ligand (4-1BBL or CD137L), which is expressed on cells such as macrophages and activated B cells. Once activated, 4-1BB functions to promote division and survival of cells, enhance the effector function of activated T cells, and generate immunological memory.

Given its pivotal role in modulating T cell function, 4-1BB and 4.-1BB agonists in particular have become an attractive target for the development of cancer immunotherapies. In fact, a number of clinical trials have examined the efficacy of different 4-1BB targeting therapies, including anti-4-1BB antibodies alone, or in combination with tumor-targeting antibodies, checkpoint inhibitors, or chemotherapy. The majority of these clinical trials have been carried out using the anti-4-1BB antibody urelumab. Developed by Bristol-Myers Squibb, urelumab is a human IgG4 antibody currently in a number of Phase 1 and Phase 2 clinical trials designed to examine efficacy in treatment of cancers, However, urelumab administration is limited to doses of 0.1 mg/kg, as higher doses resulted in severe liver toxicity (Segal et al, Clin Cancer Res, 2017 Apr. 15; 23(8):1929-1936),

Utomilumab, developed by Pfizer, is a human IgG2 antibody that is currently in various Phase 1, Phase 2 and Phase 3 clinical trials, also designed to assess efficacy in treatment of cancers. Although utomilumab does not appear to induce dose-limiting toxicities, early clinical data indicated that its effectiveness as a monotherapy did not appear to be significant (Makkouk, et al. (2016) European Journal of Cancer 54:112-119).

This background information is provided for the purpose of making known information believed by the applicant to be of possible relevance to the present disclosure. No admission is necessarily intended, nor should be construed, that any of the preceding information constitutes prior art against the claimed invention.

### SUMMARY OF THE INVENTION

Described herein are bi specific antibody constructs binding 4-1BB and tumor-associated antigens and uses thereof. One aspect of the present disclosure relates to an antibody construct comprising: a) a first 4-1BB-binding domain that binds to a 4-1BB extracellular domain (4-1BB ECD), and b) a tumor-associated antigen (TAA) antigen binding domain (TAA antigen-binding domain) that binds to a TAA, wherein the first 4-1BB-binding domain and the TAA antigen-binding domain are linked directly or indirectly to a scaffold.

Another aspect of the present disclosure relates to an antibody construct or antigen-binding fragment thereof, that specifically binds to 4-1BB, comprising: a heavy chain variable sequence comprising three CDRs and light chain variable sequence comprising three CDRs and the heavy chain variable sequence and the light chain variable sequence is from any one of variants v28726, v28727, v28728, v28730, v20022, v28700, v28704, v28705, v28706, v28711, v28712, v28713, v20036, v28696, v28697, v28698, v28701, y28702, v28703, v28707, v20023, v28683, v28684, v28685, v28686, v28687, v28688, v28689, v28690, v28691, v28692, v28694, or v28695.

Another aspect of the present disclosure relates to pharmaceutical composition comprising the antibody constructs described herein.

Another aspect of the present disclosure relates to one or more nucleic acids encoding the antibody constructs described herein.

Another aspect of the present disclosure relates to one or more vectors comprising the one or more nucleic acids encoding the antibody constructs described herein.

Another aspect of the present disclosure relates to an isolated cell comprising the one or more nucleic acids encoding the antibody constructs described herein, or the one or more vectors comprising the one or more nucleic acids.

Another aspect of the present disclosure relates to a method of preparing the antibody constructs described herein, comprising culturing the isolated cells described herein under conditions suitable for expressing the antibody construct, and purifying the antibody construct.

Another aspect of the present disclosure relates to method of treating a subject having a cancer, comprising administering to the subject an effective amount of the antibody constructs described herein.

Another aspect of the present disclosure relates to use of an effective amount of the antibody construct described herein for the treatment of cancer in a subject in need thereof.

Another aspect of the present disclosure relates to use of the antibody constructs described herein in the preparation of a medicament for the treatment of cancer.

### BRIEF DESCRIPTION OF THE FIGURES

These and other features of the claimed invention will become more apparent in the following detailed description in which reference is made to the appended drawings.

FIG. 1 A-FIG. 1E depict exemplary antibody formats. FIG. 1 A provides a representation of a naturally-occurring antibody format (FSA format); FIG. 1B provides a representation of a one-armed antibody format (OAA) where the antigen-binding domain is in the Fab format; FIG. 1C provides a representation of a one-armed antibody format (OAA) where the antigen-binding domain is in the scFv format; FIG. 1D provides a representation of a bivalent antibody format where one antigen-binding domain is in the scFv format and the other is in a Fab format (also referred to as a hybrid format); and FIG. 1E provides a representation of a bivalent antibody format where both antigen-binding domains are in the scFv format (also referred to as a dual scFv format). In FIG. 1, the Fc portion of the exemplary antibodies are identified in white, while the antigen-binding domains are identified in grey.

FIG. 2A-FIG. 2G depict a number of additional exemplary formats contemplated for the 4-1BB×TAA antibody constructs described herein. FIG. 2A provides an example of a 1×1 format, where the antibody construct comprises one 4-1BB binding domain (depicted here as 4-1BB ligand), and one TAA antigen-binding domain (depicted here in the Fab format). FIG. 2B provides an example of a 2×1 format, where the antibody construct comprises two 4-1BB antigen-binding domains (both depicted here in the Fab format), and one TAA antigen-binding domain (depicted here in the scFv format). FIG. 2C provides an example of a 2×2 format, where the antibody construct comprises two 4-1BB antigen-binding domains (both depicted here in the Fab format), and two TAA antigen-binding domains (both depicted here in the scFv format). FIG. 2D provides another example of a 1×1 format, where the antibody construct comprises one 4-1BB binding domain (depicted here in the Fab format), and one TAA antigen-binding domain (depicted here in the scFv format). FIG. 2E provides another example of a 2×1 format, where the antibody construct comprises two 4-LBB antigen-binding domains (both depicted here in the Fab format), and one TAA antigen-binding domain (depicted here in the scFv format), linked to one of the 4-1BB antigen-binding domains. FIG. 2F provides another example of a 1×1 format, where the antibody construct comprises one 4-1BB binding domain (depicted here in the Fab format), and one TAA antigen-binding domain (depicted here in the scFv format); this type of antibody construct is also referred to as a hybrid format. FIG. 2F provides another example of a 1×1 format, where the antibody construct comprises one 4-1BB binding domain (depicted here in the Fab format), and one TAA antigen-binding domain (depicted here in the scFv format), linked to the 4-1BB antigen-binding domain. The representations in FIG. 2 are exemplary only, and it is to be understood that although the 4-1BB antigen-binding domains are depicted in the Fab format here, they may also be in the scFv or sdAb format, or if there are two 4-1BB antigen-binding domains, they may be in different formats or may bind to different epitopes of 4-1BB. Likewise, although the TAA antigen-binding domains are depicted here in the scFv format, they may also be in the Fab or sdAb format, or if there are two TAA antigen-binding domains, they may be in different formats, or may bind to different TAAs.

FIG. 3 depicts the formats of exemplary 4-1BB×HER2 antibody constructs that were constructed as described in Example 1.

FIG. 4A-FIG. 41 show the ability of 4-1BB×HER2 antibody constructs in different formats and controls to activate 4-1BB in a co-culture experiment using 4-1BB-NFkB-Luciferase Jurkat reporter cells and SKOV3 or MBA-MB-468 tumour cells. Shown is the amount of luminescence induced by each antibody construct: v16675 (FIG. 4A); v16679 (FIG. 4B); v15534 (FIG. 4C); v16601 (FIG. 4D); v16605 (FIG. 4E); v19353 (FIG. 4F); v1040 (FIG. 4G); v16992 (FIG. 4H), and v12952 (FIG. 4I).

FIG. 5 shows the ability of 4-1BB×HER2 antibody constructs in different formats and controls to activate 4-1BB in a primary CD+ T cell co-culture experiment with and without SKBR3 tumour cells. Production of IL-2 by the T cells was measured by ELISA.

FIG. 6 compares the ability of 4-1BB×HER2 antibody construct v16679, the 4-1BB antibody v12592, the 4-1BB×HER2 anticalin construct v19353, and the negative control antibody v13725 to stimulate IFNγ production in an assay in which CD4⁺, CD8⁺ or pan-T cells were co-cultured with HER2⁺ SKBR3 cells.

FIG. 7 depicts the ability of chimeric 4-1BB antibodies to stimulate 4-1BB activity in a 4-1BB-NF-κB reporter gene assay when crosslinked with an anti-Fc antibody. The four columns for each variant correspond (in order right-to-left) to the concentrations of antibody construct tested: 2.5 μg/ml, 0.833 μg/ml, 0.277 μg/ml, 0.092 μg/ml.

FIG. 8A-FIG. 8B show the constructs used for domain-mapping of antibody binding to 4-1BB. FIG. 8A shows the human, dog and dog-human chimeric 4-1BB constructs; FIG. 8B shows the full-length transmembrane human 4-1BB and the truncated human domain 3 and 4 construct.

FIG. 9A-FIG. 9I show the ability of chimeric 4-1BB antibodies and controls to bind to human and dog 4-1BB. The results are shown for v12592 in FIG. 9A; for v12593 in FIG. 9B; for v20022 in FIG. 9C; for v20023 in FIG. 9D; for v20025 in FIG. 9E; for v20029 in FIG. 9F; for v20032 in FIG. 9G; for v20036 in FIG. 9H, and for v20037 in FIG. 9I.

FIG. 10 depicts the ability of chimeric antibodies to bind to various 4-1BB proteins expressed in 293E6 cells.

FIG. 11A shows the ability of chimeric antibodies to bind to cynomolgus 4-1BB. FIG. 11B depicts the ability of these chimeric antibodies to bind to mouse 4-1BF.

FIG. 12A-FIG. 12F show the sequences of the mouse heavy chain variable domain CDRs of A) 1C8, B) 1G1 and C) 5G8 ported onto a human framework, as well as mouse light chain variable domain CDRs of D) 1C8, E) 1G1 and F) 5G8 ported onto a human framework. The sequences are numbered according to Kabat and the CDRs were assigned with the AbM definition and are identified by “*”.

FIG. 13 depicts the SPR sensorgrarns of representative humanized 4-1BB antibodies derived from mouse 1C8 antibody.

FIG. 14 depicts the SPR sensorgrams of representative humanized 4-1BB antibodies derived from mouse 1G1 antibody.

FIG. 15 depicts the SPR sensorgrams of representative humanized 4-1BB antibodies derived from mouse 5G8 antibody.

FIG. 16A depicts the ability of humanized 4-1BB antibodies derived from 1C8 to bind cells expressing 4-1BB as measured by flow cytometry. FIG. 16B depicts the ability of humanized 4-1BB antibodies derived from 1G1 to bind cells expressing 4-1BB as measured by flow cytometry. FIG. 16C depicts the ability of humanized 4-1BB antibodies derived from 5G8 to bind cells expressing 4-1BB as measured by flow cytometry.

FIG. 17 depicts the DSC thermograms of humanized antibodies derived from 1C8.

FIG. 18 depicts the DSC thermograms of humanized antibodies derived from 1G1.

FIG. 19 depicts the DSC thermograms of humanized antibodies derived from 5G8.

FIG. 20 depicts the LC-MS profile of a representative purified humanized antibody derived from 1C8.

FIG. 21A shows the ability of humanized 4-1BB antibodies derived from 1C8 to stimulate 4-1BB activity in the 4-1BB-NF-κB-luc reporter assay. FIG. 21B shows the ability of humanized 4-1BB antibodies derived from 1G1 to stimulate 4-1BB activity in the 4-1BB-NF-κB-luc reporter assay. FIG. 21C shows the ability of humanized 4-1BB antibodies derived fr©m 5G8 to stimulate 4-1BB activity in the 4-1BB-NF-κB-luc reporter assay.

FIG. 22 depicts the formats of exemplary 4-1BB×TAA antibody constructs prepared as described in Example 17.

FIG. 23A depicts the ability of 4-1BB×MSLN antibody constructs v2.2329 and v22639 to stimulate 4-1BB activity in a co-culture experiments using 4-1BB-NF-κB-Luciferase Jurkat reporter cells and MSLN^{high} H226 tumour cells or MSLN^{low} A549 cells. FIG. 23B depicts the ability of 4-1BB×MSLN antibody constructs v22353 and v22630 to stimulate 4-1BB activity in the same assay.

FIG. 24 shows the ability of the 4-1BB×FRα antibody construct v22638 to stimulate 4-1BB activity in the 4-1BB-NF-κB-Luciferase Jurkat reporter cell co-culture assay.

FIG. 25A shows the ability of the 4-1BB×NaPi2b construct v22345 to enhance IFNγ production by CD8 cells when co-cultured with tumor cells expressing NaPi2b at different levels. FIG. 25B shows the ability of the 4-1BB×MSLN construct v22630 to enhance IFNγ production by CD8 cells when in co-culture with various tumor cells expressing MSLN at varying levels. FIG. 25C shows the ability of the 4-1BB×FRα construct v22638 to enhance IFNγ production by CD8 T cells when in co-culture with various tumor cells expressing FRα at varying levels. FIG. 25D shows that the control 4-1BB monospecific antibody v12592 is unable to enhance IFNγ production by CD8 T cells when in co-culture with various tumor cells expressing varying levels of TAA.

FIG. 26 shows the formats of exemplary 4-1BB×FRα antibody constructs prepared as described in Example 20. The scFv orientation depicted in this Figure is for illustration only; the scFvs in the constructs may be in VH-VL or VL-VH orientation as described in Table 11.

FIG. 27A-FIG. 27F depict the ability of 4-1BB×FRα antibody constructs to bind to 4-1BB-expressing Jurkat cells as measured by flow cytometry. FIG. 27A shows the data for antibody constructs having a 4-1BB paratope derived from mouse antibody 1C8; FIG. 27B from mouse antibody 2E8; FIG. 27C from mouse antibody 4E6; FIG. 27D from mouse antibody 5G8; FIG. 27E from mouse antibody 6B3, and FIG. 27F from antibody MOR7480.1.

FIG. 28A-FIG. 28C depict the ability of the 4-1BB×FRα antibody constructs to bind to FRα expressed on 293E cells as measured by flow cytometry. FIG. 28A depicts the ability of v23656, v23657, v23658, v23659, and v23660 to bind to cells; FIG. 28B depicts the ability of v23661, v23662, v23663, v23664, and v23665 to bind to cells, and FIG. 28C depicts the ability of v23651, v17721, and IgG1 to bind to cells.

FIG. 29A depicts the ability of 4-1BB×FRα antibody constructs having FRα paratope 8K22 to stimulate IFNγ production in a CD8+ T cell co-culture assay with FRα^{high} IGROV1 cells or FRα^{low} A549 cells. FIG. 29B depicts the ability of 4-1BB×FRα antibody constructs having FRα paratope 1H06 to stimulate IFNγ production in a CD8+ T cell co-culture assay. FIG. 29C depicts the ability of monospecific 4-1BB antibodies v20022, v20036 or v12592 and the monospecific FRα antibody v17721 to stimulate IFNγ production in the CD8+ T cell co-culture assay.

FIG. 30A and FIG. 30B depict UPLC-SEC and Caliper profiles, respectively, of purified parental chimeric 8K22 variant 23820, while FIG. 30C and FIG. 30D depict UPLC-SEC and Caliper profiles, respectively, of purified representative humanized 8K22 variant 23807.

FIG. 31A depicts the BLI sensorgrams for the parental chimeric 8K22 antibody v23820 and two representative humanized 8K22 antibodies v23801 and v23807 in the supernatant. FIG. 31B depicts the BLI sensorgrams for the parental chimeric 8K22 antibody v23820 and the two representative humanized 8K22 antibodies v23801 and v23807 after purification.

FIG. 32A depicts the DSC thermograms of purified representative humanized 8K22 antibodies exhibiting a single transition. FIG. 32B depicts the DSC thermograms of purified representative humanized 8K22 antibodies exhibiting a two-state transition.

FIG. 33A depicts the LC/MS profile of purified representative humanized 8K22 antibody v23801. FIG. 33B depicts the LC/MS profile of purified representative humanized 8K22 antibody v23807.

FIG. 34 depicts the DSC thermograms of antibodies having 8K22 binding arms.

FIG. 35A depicts the BLI sensorgram for v29675; FIG. 35B depicts the BLI sensorgram for v29677; FIG. 35C depicts the BLI sensorgram for v29680.

FIG. 36 depicts representations of the additional4-1BB×FRα bispecific antibodies prepared and tested as described in Example 33.

FIG. 37 depicts the ability of various 4-1BB×FRα bispecific antibodies to stimulate IFNγ production in a primary T cell:tumour cell co-culture assay with IGROV1 cells.

FIG. 38A depicts the ability of 4-1BB×FRα bispecific antibodies to activate 4-1BB in an NFκB reporter gene assay with IGROV1 cells. FIG. 38B depicts the ability of 4-1BB×FRα bispecific antibodies to activate 4-1BB in an NFκB reporter gene assay with A431 cells. FIG. 38C depicts the ability of 4-1BB×FRα bispecific antibodies to activate 4-1BB in an NFκB reporter gene assay with HCC827 cells. FIG. 38D depicts the ability of 4-1BB×FRα bispecific antibodies to activate 4-1BB in an NFκB reporter gene assay with OVKATE cells. FIG. 38E depicts the ability of 4-1BB×FRα bispecific antibodies to activate 4-1BB in an NFκB reporter gene assay with OVCAR3 cells. FIG. 38F depicts the ability of 4-1BB×FRα bispecific antibodies to activate 4-1BB in an NFκB reporter gene assay with H661 cells. FIG. 38G depicts the ability of 4-1BB×FRα bispecific antibodies to activate 4-1BB in an NFκB reporter gene assay with H441 cells. FIG. 38H depicts the ability of 4-1BB×FRα bispecific antibodies to activate 4-1BB in an INFκB reporter gene assay with H1975 cells.

FIG. 39A demonstrates the ability of the mouse anti 4-1BB paratope 1C8 to bind to cyno 4-1BB; FIG. 39D demonstrates the ability of the humanized anti-4-1BB paratope 1C8 to bind to cyno 4-1BB FIG. 39B demonstrates the ability of the mouse anti 4-1BB paratope 1G1 to bind to cyno 4-1BB; FIG. 39E demonstrates the ability of the humanized anti-4-1BB paratope 1G1 to bind to cyno 4-1BB. FIG. 39C demonstrates the ability of the mouse anti 4-1BB paratope 5G8 to bind to cyno 4-1BB; FIG. 39F demonstrates the ability of the humanized anti-4-1BB paratope 5G8 to bind to cyno 4-1BB.

FIG. 40A shows the sequence of the rabbit heavy chain variable domain CDRs of 8K22 ported onto a human framework; FIG. 40B shows the sequence of the rabbit light chain variable domain CDRs of 8K22 ported onto a human framework. The sequences are numbered according to Kabat and the CDRs were assigned with the AbM definition and are identified by “*”.

### DETAILED DESCRIPTION

The present disclosure relates to 4-1BB×TAA antibody constructs that specifically hind to a 4-1BB extracellular domain (ECD) and to a tumor-associated antigen (TAA). In some embodiments, the TAA may be a folate receptor-α (FRα), Solute Carrier Family 34 Member 2 (SLC34A2 or NaPi2b), FEER2, mesothelin (MSLN), or Solute Carrier Family 39 Member 6 (SLC39A6 or LIV-1). In some embodiments, the 4-1BB×TAA antibody constructs may be capable of conditionally enhancing the activity of T cells within a tumor. In some embodiments, the 4-1BB×TAA antibody constructs are capable of promoting conditional agonism of 4-1BB. In some embodiments, the 4-1BB×TAA antibody constructs may be more effective in activating 4-1BB on T cells in the presence of TAA-expressing cells compared to a monospecific, monovalent anti-4-1BB antibody, as measured by cytokine production. In some embodiments, the 4-1BB×TAA antibody constructs may be more effective in activating 4-1BB on T cells in the presence of tumor cells expressing the TAA at medium to high levels compared to in the presence of tumor cells expressing the TAA at low levels. Thus, in related embodiments, the 4-1BB×TAA antibody constructs may be used to treat cancer.

The present disclosure further provides antibody sequences that specifically bind 4-1BB (anti-4-1BB antibody sequences). These anti-4-1BB antibody sequences may be used in the preparation of monospecific, bispecific, or multispecific antibody constructs that bind to 4-1BB (4-1BB antibody constructs). These monospecific, bispecific, or multispecific 4-1BB antibody constructs may also be used in the treatment of cancer, either alone or in combination with other anti-cancer therapies.

### Definitions

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±1%, 2%, 3%, 4%, 5%, 6%, 7%, 8%, 9% or 10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives.

As used herein, the terms “include,” “have,” “contain,” “comprise,” and grammatical variations thereof are used synonymously. These terms are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is also to be understood that the positive recitation of a feature in one embodiment serves as a basis for excluding the feature in a particular embodiment. For example, where a list of options is presented for a given embodiment or claim, it is to be understood that one or more option may be deleted from the list and the shortened list may form an alternative embodiment, whether or not such an alternative embodiment is specifically referred to.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose

It is to be understood that the methods and compositions described herein are not limited to the particular methodology, protocols, cell lines, constructs, and reagents described herein and as such may vary. It is also to be understood that the terminology used herein is for the purpose of describing particular embodiments only and is not intended to limit the scope of the methods and compositions described herein.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

In the present application, amino acid names and atom names (e.g. N, O, C, etc.) are used as defined by the Protein DataBank (PDB) (www.pdb.org), which is based on the IUPAC nomenclature (OUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). The term “amino acid residue” is primarily intended to indicate an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al, Eds., Antibodies: A Laboratory Manual, Chapter 14 (Cold Spring Harbor Laboratory, Cold Spring Harbor, 1988).

As used herein, the terms “antibody” and “immunoglobulin” are used interchangeably. An “antibody” refers to a polypeptide substantially encoded by an immunoglobulin gene or immunoglobulin genes, or one or more fragments thereof, which specifically bind an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin isotypes, IgG, IgA, IgD, and IgE, respectively. Further, the antibody can belong to one of a number of subtypes, for instance, the human IgG can belong to the IgG1, IgG2, 1gG3, or IgG4 subtypes.

An exemplary immunoglobulin (antibody) structural unit is composed of two pairs of polypeptide chains, each pair having one immunoglobulin “light” (about 25 kD) and one immunoglobulin “heavy” chain (about 50-70 kD). This type of immunoglobulin or antibody structural unit is considered to be “naturally occurring,” or in a “naturally occurring format.” The term “light chain” includes a full-length light chain and fragments thereof having sufficient variable domain sequence to confer binding specificity. A full-length light chain includes a variable domain, VL, and a constant domain, CL. The variable domain of the light chain is at the amino-terminus of the polypeptide. Light chains include kappa chains and lambda chains. The term “heavy chain” includes a full-length heavy chain and fragments thereof having sufficient variable region sequence to confer binding specificity. A full-length heavy chain includes a variable domain, VH, a hinge region, and constant domains, CH1, CH2, and CH3, optionally a CH4 domain. The VH domain is at the amino-terminus of the polypeptide, and the CH domains are at the carboxyl-terminus, with the CH3 (or CH4 where present) domain being closest to the carboxy-terminus of the polypeptide. Heavy chains can be of any isotype, including IgG (including IgG2, IgG3 and IgG4 subclasses), IgA (including IgA1 and IgA2 subclasses), IgM, IgD and IgE. The term “variable region” or “variable domain” refers to a portion of the light and/or heavy chains of an antibody generally responsible for antigen recognition, typically including approximately the amino-terminal 120 to 130 amino acids in the heavy chain (VH) and about 100 to 110 amino terminal amino acids in the light chain (VL).

The terms “antigen,” “immunogen,” “antibody target,” “target analyte,” and like terms are used herein to refer to a molecule, compound, or complex that is recognized by an antibody, i.e. can be specifically bound by the antibody. The term can refer to a molecule that can be specifically recognized by an antibody, e.g., a polypeptide, polynucleotide, carbohydrate, lipid, chemical moiety, or combinations thereof (e.g., phosphotylated or glycosylated polypeptides, etc.). One of skill will understand that the term does not indicate that the molecule is immunogenic in every context, but simply indicates that it can be targeted by an antibody.

An “antigen-binding domain” is that portion of an antibody that is capable of specifically binding to an epitope or antigen. The epitope- or antigen-binding function of an antibody can be performed by fragments of an antibody in a naturally occurring format. Examples of antigen-binding domains include (i) a Fab fragment, a monovalent fragment consisting of the VH, VL, CH1 and CL domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VH and VL domains of a single arm of an antibody, (v) a sdAb fragment (Ward et al., (1989) Nature 341:544-546), which comprises a single variable domain; and (vi) an isolated complementarity determining region (CDR). Exemplary formats of the antigen-binding domains described herein include but are not limited to the Fab, scFv, VHH, or sdAb formats. Furthermore, methods of converting between types of antigen-binding domains are known in the art (see, for example, methods for converting an scFv to a Fab format described in Zhou et al (2012) Mol Cancer Ther 11:1167-1476). Thus, if an antibody is available in a format that includes an antigen-binding domain that is an scFv, but it is desired that the antibody construct comprise an antigen-binding domain in a Fab format, one of skill in the art would be able to make such conversion, and vice-versa.

A “Fab fragment” (also referred to as fragment antigen-binding, Fab format) includes the constant domain (CL) sequences of the light chain and the constant domain 1 (CH1) of the heavy chain along with the variable domains VL and VH on the light and heavy chains, respectively. The variable domains comprise the CDRs, which are involved in antigen-binding. Fab′ fragments differ from Fab fragments by the addition of a few amino acid residues at the C-terminus of the heavy chain CH1 domain, including one or more cysteines from the antibody hinge region.

A “single-chain Fv” or “scFv” format includes the VH and VL domains of an antibody in a single polypeptide chain. The scFv polypeptide may optionally further comprise a polypeptide linker between the VH and VL domains which enables the scFv to form a desired structure for antigen binding. For a review of says see Pluckthun in The Pharmacology of Monoclonal Antibodies, vol. 113, Rosenburg and Moore eds., Springer-Verlag, New York, pp. 269-315 (1994).

A “single domain antibody” or “sdAb” format refers to a single immunoglobulin domain. The sdAb may be, for example, of camelid origin. Camelid antibodies lack light chains and their antigen-binding sites consist of a single domain, termed a “VHH.” An sdAb comprises three CDR/hypervariable loops that form the antigen-binding site: CDR1, CI)R2 and CDR3. SdAbs are fairly stable and can be expressed as a fusion with the Fc region of an antibody (see, for example, Harmsen M M, De Haard H J (2007) “Properties, production, and applications of camelid single-domain antibody fragments,” Appl. Microbiol Biotechnol. 77(1): 13-22).

Antibodies bind to an “epitope” on an antigen. The epitope is the localized site on the antigen that is recognized and bound by the antibody. Epitopes can include a few amino acids, e.g., 5 or 6, or more, e.g., 20 or more amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

An epitope may be determined by obtaining an X-ray crystal structure of an antibody:antigen complex and determining which residues on the antigen are within a specified distance of residues on the antibody of interest, wherein the specified distance is, 5 Å or less, e.g., 5 Å, 4 Å, 3 Å, 2 Å, 1 Å or less, or any distance in between. In some embodiments, the epitope is defined as a stretch of 8 or more contiguous amino acid residues along the antigen sequence in which at least 50%, 70% or 85% of the residues are within the specified distance of the antibody or binding protein in the X-ray crystal structure. Mapping of epitopes recoginzed by antibodies can also be performed as described in detail in “Epitope Mapping Protocols” (Methods in Molecular Biology) by Glenn E. Morris ISBN-089603-375-9 (1996), and in “Epitope Mapping: A Practical Approach” Practical Approach Series, 248 by Olwyn M. R. Westwood, Frank C. Hay, (2001). For exam pie, X-ray co-crystallography cryogenic electron microscopy, array-based oligo-peptide scanning, site-directed mutagenesis mapping, hydrogen-deuterium exchange, cross-linking coupled mass spectrometry, may be used to determine or map epitopes. These methods are well-known in the art.

The term “specifically binds” as used herein, refers to a binding agent's ability to discriminate between possible partners in the environment in which binding is to occur. A binding agent may be an antibody, antibody construct or antigen-binding domain, for example. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations. The term “specifically binds” as used herein in relation to antigen-binding domains, antibodies or antibody constructs means that the antigen-binding domains, antibodies or antibody constructs bind to their target antigen with no or insignificant binding to other antigens.

A “complementarity determining region” or “CDR” is an amino acid sequence that contributes to antigen-binding specificity and affinity. “Framework” regions (FR) can aid in maintaining the proper conformation of the CDRs to promote binding between the antigen-binding region and an antigen. Structurally, framework regions can be located in antibodies between CDRs. The variable regions typically exhibit the same general structure of relatively conserved framework regions (FR) joined by three hyper variable regions, also known as CDRs. The CDRs from the variable domains of the heavy chain and light chain typically are aligned by the framework regions, which can enable binding to a specific epitope. From N-terminal to C-terminal, both light and heavy chain variable domains typically comprise the domains FR1, CDR1, FR2, CDR2, FR3, CDR3, and FR4. The assignment of amino acids to each domain is typically in accordance with the definitions of Kabat Sequences of Proteins of Immunological Interest (National Institutes of Health, Bethesda, Md. (1987 and 1991)), unless stated otherwise. Typically, there are three heavy chain and three light chain CDRs (or CDR regions) in the variable portion of an immunoglobulin. The three heavy chain CDRs are referred to herein as HCDR1, HCDR2, and HCDR3, while the three light chain CDRs are referred to as LCDR1, LCDR2, and LCDR3. Thus, “CDRs” as used herein may refer to all three heavy chain CDRs, or all three light chain CDRs (or both all heavy and all light chain CDRs, if appropriate). CDRs provide the majority of contact residues for the binding of the antibody to the antigen or epitope. Often, the three heavy chain CDRs and the three light chain CDRs are required to bind antigen. However, in some instances, even a single variable domain can confer binding specificity to the antigen. Furthermore, as is known in the art, in some cases, antigen-binding may also occur through a combination of a minimum of one or more CDRs selected from the VH and/or VL domains, for example HCDR3.

A number of different definitions of the CDR sequences are in common use, including those described by Kabat et al, (1983, Sequences of Proteins of Immunological Interest, NIH Publication No. 369-847, Bethesda, Md.), by Chothia et al. (1987. J Mol Biol, 196:901-917), as well as the IMGT, AbM (University of Bath) and Contact (MacCallum R. M., and Martin A. C. R. and Thornton J. M, (1996), Journal of Molecular Biology, 262 (5), 732-745) definitions. By way of example, CDR definitions according to Kabat, Chothia, IMGT, AbM and Contact are provided in Table A below. Accordingly, as would be readily apparent to one skilled in the art, the exact numbering and placement of CDRs may differ based on the numbering system employed. However, it is to be understood that the disclosure herein of a VH includes the disclosure of the associated (inherent) heavy chain CDRs (HCDRs) as defined by any of the known numbering systems. Similarly, disclosure herein of a VH includes the disclosure of the associated (inherent) heavy chain CDRs (HCDRs) as defined by any of the known numbering systems.

TABLE A
Common CDR Definitions¹

| | Heavy Chain | | | Light Chain | | |
|---|---|---|---|---|---|---|
| Definition | CDR1² | CDR2 | CDR3 | CDR1 | CDR2 | CDR3 |
| Kabat | H31-H35B | H50-H65 | H95-H102 | L24-L34 | L50-L56 | L89-L97 |
| Chothia | H26-H32, | H52-H56 | H95-H102 | L24-L34 | L50-L56 | L89-L97 |
| | H33 or H34 | | | | | |
| IMGT | H26-H33, | H51-H57 | H93-H102 | L27-L32 | L50-L52 | L89-L97 |
| | H34, H35, | | | | | |
| | H35A or H35B | | | | | |
| AbM | H26-H35B | H50-H58 | H95-H102 | L24-L34 | L50-L56 | L89-L97 |
| Contact | H30-H35B | H47-H58 | H93-H101 | L30-L36 | L46-L55 | L89-L96 |

¹Either the Kabat or Chothia numbering system may be used for HCDR2, HCDR3 and the light chain CDRs for all definitions except Contact, which uses Chothia numbering
²Using Kabat numbering. The position in the Kabat numbering scheme that demarcates the end of the Chothia and IMGT CDR-H1 loop varies depending on the length of the loop because Kabat places insertions outside of those CDR definitions at positions 35A and 35B. However, the IMGT and Chothia CDR-H1 loop can be unambiguously defined using Chothia numbering. CDR-H1 definitions using Chothia numbering: Kabat H31-H35, Chothia H26-H32, AbM H26-H35, IMGT H26-H33, Contact H30-H35.

Throughout this specification, amino acid residues in VH and VL sequences are numbered according to the Kabat scheme, unless otherwise indicated.

“Chimeric antibody,” as used herein, refers to an antibody whose amino acid sequence includes VH and VL sequences that are found in a first species and constant domain sequences that are found in a second species, different from the first species. In many embodiments, a chimeric antibody has murine VH and VL sequences linked to human constant domain sequences.

“Humanized” forms of non-human (e.g., rodent) antibodies are antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fe), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“CDR-grafted antibody,” or “CDR-ported antibody” as used herein, refers to an antibody whose amino acid sequence comprises heavy and light chain variable region sequences from one species but in which the sequences of one or more of the CDR sequences of VH and/or VL sequences are replaced with CDR sequences of another species, such as antibodies having mouse VH and VL sequences in which one or more of the mouse CDRs (e.g., HCDR3) has been replaced with human CDR sequences. Likewise, a “CDR-grafted antibody” may also refer to antibodies having human VH and VL regions in which one or more of the human CDRs (e.g., CDR3) has been replaced with mouse CDR sequences.

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The alternative, where the binding of the first antibody to the target is also detectably decreased in the presence of the second antibody, can, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present disclosure. The term “competitor” antibody can be applied to the first or second antibody as can be determined by one of skill in the art. In some cases, the presence of the competitor antibody (e.g., the first antibody) reduces binding of the second antibody to the target by at least 10%, e.g., at least any of 20%, 30%, 40%, 50%, 60%, 70%, 80%, or more, e.g., so that binding of the second antibody to target is undetectable in the presence of the first (competitor) antibody.

The term “dissociation constant (K_{D} or K_{d})” as used herein, is intended to refer to the equilibrium dissociation constant of a particular ligand-protein interaction. As used herein, ligand-protein interactions refer to, but are not limited to protein-protein interactions or antibody-antigen interactions. The K_{D} measures the propensity of two proteins complexed together (e.g. AB) to dissociate reversibly into constituent components (A+B), and is defined as the ratio of the rate of dissociation, also called the “off-rate (k_{off})”, to the association rate, or “on-rate (kₒₙ)”. Thus, K_{D} equals k_{off}/kₒₙ and is expressed as a molar concentration (M). It follows that the smaller the K_{D}, the stronger the affinity of binding, and thus a decrease in K_{D} indicates an increase in affinity. Therefore, a K_{D} of 1 mM indicates weak binding affinity compared to a K_{D} of 1 nM. Affinity is sometimes measured in terms of a K_{A} or Kₐ, which is the reciprocal of the K_{D} or K_{d}. K_{D} values for antigen-binding constructs can be determined using methods well established in the art. One method for determining the K_{D} of an antigen-binding construct is by using surface plasmon resonance (SPR), typically using a biosensor system such as a Biacore® system. Isothermal titration calorimetry (ITC) is another method that can be used to measure K_{D}. The Octet™ system may also be used to measure the affinity of antibodies for a target antigen.

As used herein, the term “conditional agonism” is intended to refer to the ability of the 4-1BB×TAA antibody constructs to agonize 4-1BB activity in immune cells such as for example, T cells or NK cells, predominantly when the immune cells are in the proximity of TAA expressing cells. In one embodiment, conditional agonism” refers to the ability of the 4-1BB×TAA antibody constructs to agonize 4-1BB activity in immune cells only when the immune cells are in the proximity of TAA expressing cells.

The term “amino acid modifications” as used herein includes, but is not limited to, amino acid insertions, deletions, substitutions, chemical modifications, physical modifications, and rearrangements. In some embodiments, the amino acid modification is an amino acid substitution.

The amino acid residues for the immunoglobulin heavy and light chains may be numbered according to several conventions including Kabat (as described in Kabat and Wu, 1991; Kabat et al, Sequences of proteins of immunological interest. 5th Edition-US Department of Health and Human Services, NIH publication no. 91-3242, p 647 (1991)), IMGT (as set forth in Lefranc, M.-P., et al. IMGT®, the international ImMunoGeneTics information system® Nucl. Acids Res, 37, D1006 D1012 (2009), and Lefranc, M.-P., IMGT, the International ImMunoGeneTics Information System, Cold Spring Harb Protoc. 2011 Jun. 1; 2011(6)), 1JPT (as described in Katja Faelber, Daniel Kirchhofer, Leonard Presta, Robert F Kelley, Yves A Muller. The 1.85 Å resolution crystal structures of tissue factor in complex with humanized fab d3h44 and of free humanized fab d344: revisiting the solvation of antigen combining sites1, Journal of Molecular Biology, Volume 313, Issue 1, Pages 83-97,) and EU (according to the EU index as in Kabat referring to the numbering of the EU antibody (Edelman et al., 1969, Proc Natl Acad Sci USA 63:78-85)). Kabat numbering is used herein for the VH, CH1, CL, and VL domains unless otherwise indicated. EU numbering is used herein for the CH3 and CH2 domains, and the hinge region unless otherwise indicated.

### Antibody Constructs

“Antibody construct,” as used herein, refers to a polypeptide or a set of polypeptides that specifically bind to an epitope or antigen and include one or more immunoglobulin structural features. In general, an antibody construct is a polypeptide or set of polypeptides whose amino acid sequence includes elements characteristic of an antigen-binding domain (e.g., an antibody light chain or variable region or one or more complementarity determining regions (“CDRs”) thereof, or an antibody heavy chain or variable region or one more CDRs thereof, optionally in presence of one or more framework regions). In some embodiments, an antibody construct is or comprises an antibody in a naturally occurring format. In some embodiments, the term “antibody construct” encompasses a protein having a binding domain which is homologous or largely homologous to an immunoglobulin-binding domain. In some embodiments, an antibody construct comprises a fragment of a naturally occurring antibody including at least one antigen-binding domain. In some embodiments, the antibody construct may further comprise a binding domain that is other than an antigen-binding domain, for example a ligand for a target protein.

In particular embodiments, an “antibody construct” encompasses polypeptides having an antigen-binding domain that shows at least 99% identity with an immunoglobulin binding domain. In some embodiments, an “antibody construct” is any polypeptide having a binding domain that shows at least 70%, 75%, 80%, 85%, 90%, 95% or 98% identity with an immunoglobulin binding domain, for example a reference immunoglobulin binding domain. An “antibody construct” may have an amino acid sequence identical to that of an antibody (or a fragment thereof, e.g., an antigen-binding fragment thereof) that is found in a natural source. An “antigen-binding fragment” of an antibody includes a fragment of an antibody having an antigen-binding domain with the required specificity. Thus, an antigen-binding fragment includes antibody fragments, derivatives, functional equivalents and homologues of antibodies, humanized antibodies, including any polypeptide comprising an immunoglobulin binding domain, whether natural or wholly or partially synthetic Chimeric molecules comprising an immunoglobulin binding domain, or equivalent, fused to another polypeptide are also included.

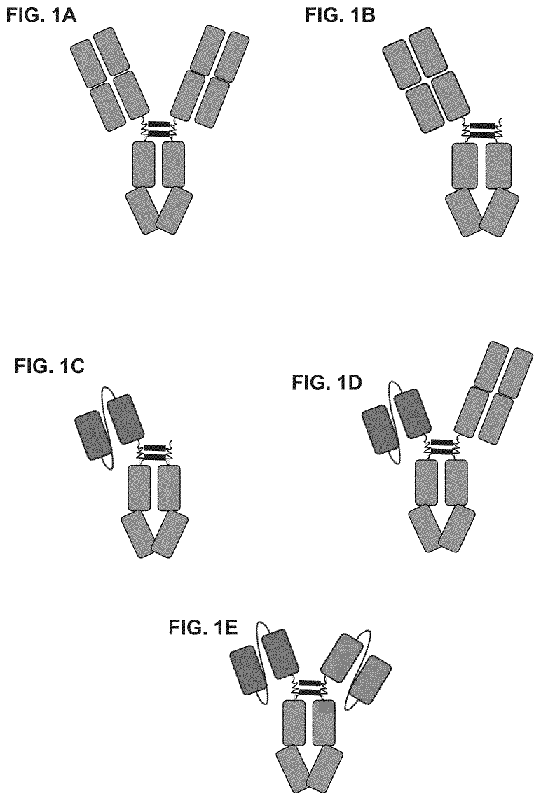

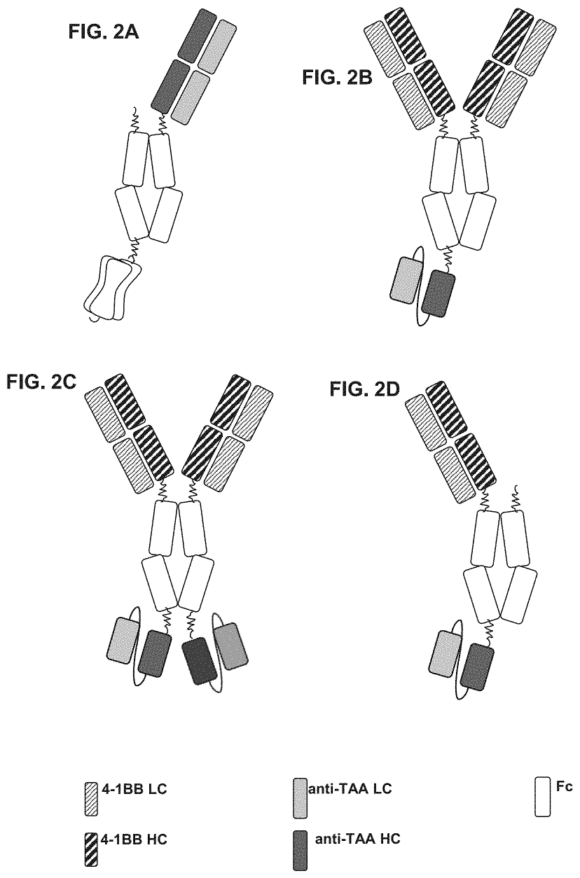

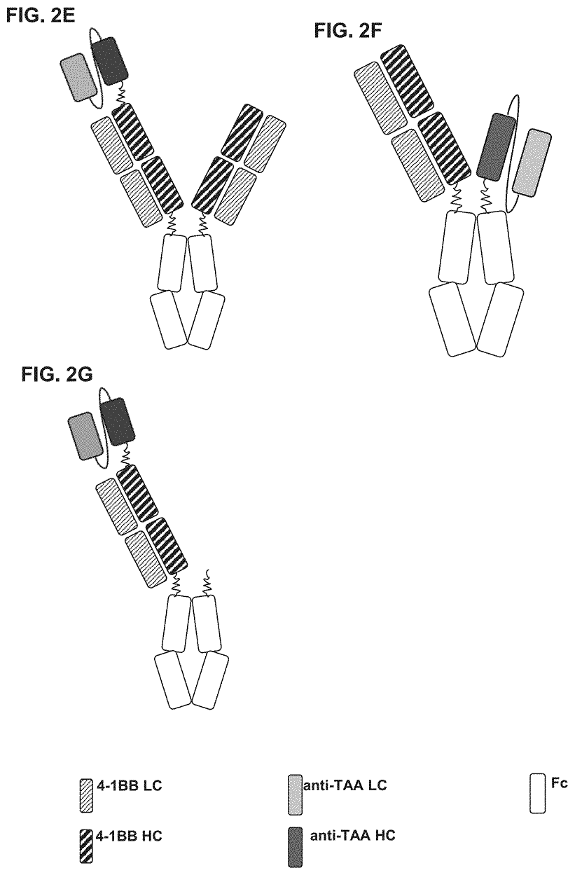

An antibody construct may be monospecific, bi specific, or multispecific, and may bind to at least one distinct target, antigen or epitope. Antibody constructs can be prepared in many formats, and exemplary antibody construct formats are described in FIGS. 1 and 2, and elsewhere throughout the application. The term “antibody construct” as used herein is meant to encompass monospecific, bispecific, or multispecific antibody constructs. A “monospecific” antibody construct is a species of antibody construct that binds to one target, antigen, or epitope. Monospecific antibody constructs may comprise one or more antigen-binding domains, each binding to the same epitope. Monospecific antibody constructs may be monovalent (i.e. having only one arm or paratope), bivalent (i.e. having two arms or paratopes, both binding to the same epitope) or multivalent (i.e. having multiple arms or paratopes, all binding to the same epitope). A “bispecific” antibody construct is a species of antibody construct that targets two different antigens or epitopes. In general, a bispecific antibody construct can have two antigen-binding domains, although, in some embodiments, a bispecific antibody construct may have more than two antigen-binding domains, provided that no more than two unique epitopes are recognized by the antigen-binding domains. The two or more antigen-binding domains of a bispecific antibody construct will bind to two different epitopes, which can reside on the same or different molecular targets. Where the two different epitopes reside on the same molecular target, the bispecific antibody construct is referred to herein as “biparatopic.” In some embodiments, the monospecific or bispecific antibody constructs are in a naturally occurring format, also referred to herein as a full-sized (FSA) format. In other words, in the latter embodiment, the monospecific or bispecific antibody construct has the same format as a naturally occurring IgG, IgA, IgD, or IgE antibody.

A multispecific antibody construct can include three or more antigen-binding domains, each capable of binding to a different target or epitope. In some embodiments, the multispecific antibody construct comprises a format that is the same as a naturally occurring IgG, IgA, IGM, IgD, or IgE antibody, but further includes one or more additional antigen-binding domains.

In some embodiments, an antibody construct may have structural elements characteristic of chimeric or humanized antibodies or may have amino acid sequences derived from chimeric or humanized antibodies. In some embodiments, an antibody construct may have structural elements characteristic of a human antibody.

Described herein are antibody constructs capable of binding to the extracellular domain (M) of 4-1BB and to a tumor-associated antigen (TAA). Also described herein are antibody constructs comprising sequences that specifically bind to the ECD of 4-1BB.

Antibody Constructs that Bind to 4-1BB and to a TAA (4-1BB×TAA Antibody Constructs)

Antibody constructs capable of binding to the ECD of 4-1BB and to a TAA comprise a 4-1BB binding domain that binds to a 4-1BB ECD and a TAA antigen-binding domain, wherein the 4-1BB binding domain and the TAA antigen-binding domain are linked directly or indirectly to a scaffold. Accordingly, in certain embodiments, the 4-1BB×TAA antibody constructs described herein are bispecific antibody constructs that bind to two distinct targets. In certain other embodiments, the 4-1BB×TAA antibody construct may be a multi specific antibody construct where the 4-1BB×TAA antibody construct binds to 4-1BB and to two or more distinct TAAs. In related embodiments, the scaffold is an Fc construct. In certain embodiments, the scaffold is an Fc construct with modifications that reduce its ability to mediate effector function.

In some embodiments, the 4-1BB×TAA antibody constructs are capable of binding to 4-1BB-expressing cells. In some embodiments, the 4-1BB×TAA antibody constructs are capable of binding to TAAs expressed on the surface of cancer cells. In some embodiments, the 4-1BB×TAA antibody constructs are capable of activating 4-1BB signalling in 4-1BB-expressing cells. In some embodiments, the 4-1BB×TAA antibody constructs are capable of enhancing CD3-stimulated T cell activation.

### 4-1BB-Binding Domains

4-1BB (also known as TNFRSF9 or CD137) is a member of the TNF receptor superfamily. Human 4-1BB is a 255 amino acid protein (Accession Nos. NM_001561 and UniProt Q07011 for mRNA and polypeptide sequences respectively). The complete human 4-1BB amino acid sequence is provided in SEQ ID NO:79. The sequence shown in SEQ ID NO:1 includes a signal sequence (amino acid residues 1-23), an extracellular domain (ECD, amino acid residues 23-187), a transmembrane region (amino acids 188 to 213), and an intracellular domain (amino acids 214 to 255) (Bitra. et al. J. Biol. Chem. (2018) 293(26) 9958 9969).

The 4-1BB receptor is expressed on the cell surface in monomeric and dimeric forms and likely trimerizes with 4-1BB ligand to allow signalling. The structure of mammalian 4-1BB protein consists of four Cysteine-Rich Domains (CRDs) which show homology to other TNFR superfamily members. CRD1 consists of amino acids 24 to 45, and both the mouse and human 4-1BB lack a disulphide found in other TNFR superfamily members. CRD2 and CRD3 extend from amino acids 47 to 86 and 87 to 118, respectively, and are the domains that contact 4-1BBL (Bitra et al, supra). CRD4 is comprised of amino acids 119 to 159 and is followed by a short stalk region comprised on amino acids 160 to the transmembrane domain at amino acid 187 (with reference to SEQ ID NO:79). CRD1, CRD2, CRD3 and CDR4 are also referred to herein as domains 1, 2, 3, and 4, respectively.

In one embodiment, the 4-1BB×TAA antibody construct comprises one 4-1BB binding domain. In some embodiments, the 4-1BB×TAA antibody construct may comprise more than one 4-1BB-binding domain. In certain embodiments, the 4-1BB×TAA antibody construct comprises two 4-1BB-binding domains, and a TAA antigen-binding domain, wherein the 4-1BB-binding domain and the TAA antigen-binding domain are linked directly or indirectly to a scaffold. In related embodiments, the 4-1BB×TAA antibody construct comprises two 4-1BB-binding domains, and a TAA antigen-binding domain, wherein the 4-1BB-binding domain and the TAA antigen-binding domain are linked directly or indirectly to a scaffold, and wherein at least one of the 4-1BB-binding domains is a 4-1BB antigen-binding domain. In related embodiments, where the 4-1BB×TAA antibody construct comprises two or more 4-1BB antigen-binding domains, each 4-1BB antigen-binding domain may bind to the same epitope of the ECD of 4-1BB, or they may bind to different epitopes of the ECD of 4-1BB. In other embodiments, the 4-1BB×TAA antibody construct comprises a 4-1BB binding domain that is an antigen-binding domain, and a TAA antigen-binding domain.

In yet other embodiments, the 4-1BB×TAA antibody construct comprises three or more 4-1BB-binding domains that bind to the ECD of 4-1BB. In one embodiment, the three or more 4-1BB-binding domains include at least one 4-1BB antigen-binding domain. In one embodiment, the three or more 4-1BB-binding domains include at least two 4-1BB antigen-binding domains. In the latter embodiment, the two 4-1BB antigen-binding domains may bind to the same epitope of 4-1BB, or they may bind to different epitopes of 4-1BB.

The 4-1BB antigen-binding domains may be in say, Fab or sdAb formats. Thus, in one embodiment, the 4-1BB antigen-binding domain of the 4-1BB×TAA antibody construct is in a Fab format. In alternate embodiments, the 4-1BB antigen-binding domain is in a scFv format. In additional embodiments, the 4-1BB×TAA antibody construct comprises more than one 4-1BB antigen-binding domain, wherein at least one 4-1BB antigen-binding domain is in a Fab format. In other embodiments where the 4-1BB×TAA comprises more than one 4-1BB antigen-binding domain, at least two of the antigen-binding domains are in the Fab format.

The 4-1BB×TAA antibody construct comprises a 4-1BB-binding domain that binds to the ECD of 4-1BB. In certain embodiments, the 4-1BB×TAA antibody construct comprises a 4-1BB-binding domain that can bind to an ECD of human 4-1BB. Suitable 4-1BB-binding domains include naturally occurring molecules such as ligands or 4-1BB-binding fragments thereof. Examples of such molecules include 4-1BB ligand (see NP_003802.1, for example), also known as TNFSF9 or CD137L. Thus, in one embodiment, the antibody construct comprises a 4-1BB-binding domain that binds to a 4-1BB ligand and a TAA antigen-binding domain, wherein the 4-1BB-binding domain and the TAA antigen-binding domain are linked directly or indirectly to a scaffold.

As indicated above, in some embodiments, the 4-1BB×TAA antibody construct comprises a 4-1BB-binding domain that is an antigen-binding domain. Antigen-binding domains can be constructed from the sequences of antibodies that bind to the ECD of 4-1BB. Suitable antibodies include those that are known in the art, commercially available, or those that are identified and prepared according to methods well known in the art and described herein. 4-1BB antigen-binding domains may be constructed from mouse, human, humanized, or chimeric anti-4-1BB antibodies. In some embodiments, the 4-1BB antigen-binding domain is derived from an agonistic anti-4-1BB antibody, Agonistic anti-4-1BB antibodies bind to 4-1BB and are able to stimulate 4-1BB signalling activity. 4-1BB signalling activity refers to at least one of the activities that can be exhibited by 4-1BB in vitro or in vivo. For example, these activities may include stimulation of cytokine release from T or NK cells, or an increase in metabolic activity by T or NK cells, or enhancement of cytotoxic activity by T or NK cells.

Numerous antibodies that bind human 4-1BB are known in the art, for example and not limited to, utomilumab (described in WO2012/032433, Pfizer), urelumab (described in WO2004/010947 and WO2005/035584, BMS), and the antibodies described in WO 2018/156740 (Macrogenics), U.S. Pat. No. 8,337,850 (Pfizer), US 2018/0258177 (Eutilex) WO2017/077085 (Cancer Research Technologies), and WO2006126835 (University of Ulsan). Urelumab and utomilumab are exemplary agonistic anti-4-1BB antibodies.

In one embodiment, the 4-1BB×TAA antibody construct comprises a 4-1BB antigen-binding domain that can compete with one of the antibodies described in the preceding paragraph for binding to an epitope of 4-1BB ECD. In one embodiment, the 4-1BB×TAA antibody construct comprises a 4-1BB antigen-binding domain that can compete with utomilumab for binding to an epitope of 4-1BB ECD. In another embodiment, the 4-1BB×TAA antibody construct comprises a 4-1BB antigen-binding domain that can compete with urelumab for binding to an epitope of 4-1BB ECD, In yet another embodiment, the 4-1BB×TAA antibody construct comprises a 4-1BB antigen-binding domain that can compete with the anti-4-1BB antibodies described in US 8,337,850 for binding to an epitope of 4-1BB ECD. In a still further embodiment, the 4-1BB×TAA antibody construct comprises a 4-1BB antigen-binding domain that can compete with the anti-4-1BB antibodies described in US 2018/0258177 for binding to an epitope of 4-1BB ECD. In a still further embodiment, the 4-1BB×TAA antibody construct comprises a 4-1BB antigen-binding domain that can compete with the anti-4-1BB antibodies described in WO2018/156740 for binding to an epitope of 4-1BB ECD. In other embodiments, the 4-1BB×TAA antibody construct comprises a 4-1BB antigen-binding domain that binds to the same epitope of 4-1BB ECD as utomilumab, or urelumab, or any one of the anti-4-1BB antibodies described in U.S. Pat. No. 8,337,850, US 2018/0258177 or WO2018/156740.

In one embodiment, the 4-1BB×TAA antibody construct comprises a 4-1BB antigen-binding domain that binds to a 4-1BB ECD other than domain 3 or domain 4. In another embodiment, the 4-1BB×TAA antibody construct comprises a 4-1BB antigen-binding domain that binds to an epitope at least partially within amino acid residues 24-85 of the mature 4-1BB protein (SEQ ID NO:79). In one embodiment, the 4-1BB×TAA antibody construct comprises a 4-1BB antigen-binding domain that binds to domain 1 of 4-1BB. In another embodiment, the 4-1BB×TAA antibody construct comprises a 4-1BB antigen-binding domain that binds to domain 2 of 4-1BB. In one embodiment, the 4-1BB×TAA antibody construct comprises a 4-1BB antigen-binding domain that binds to domain 3 of 4-1BB. In one embodiment, the 4-1BB×TAA antibody construct comprises a 4-1BB antigen-binding domain that binds to domain 4 of 4-1BB.

In some embodiments, the 4-1BB×TAA antibody construct comprises a 4-1BB antigen-binding domain that binds to the ECD of human and cynomolgus 4-1BB.

In one embodiment, the 4-1BB×TAA antibody construct comprises a 4-1BB antigen-binding domain that comprises at least one, two, or all three heavy chain CDRs and/or at least one, two, or all three light chain CDRs of urelumab, utomilumab, or any one of the anti-4-1BB antibodies described in U.S. Pat. No. 8,337,850, US 2018/0258177 or WO2018/156740. In an alternate embodiment, the 4-1BB×TAA antibody construct comprises a 4-1BB antigen-binding domain that comprises at least one, two, or all three heavy chain CDRs and at least one, two, or all three light chain CDRs of urelumab, utomilumab, or any one of the anti-4-1BB antibodies described in U.S. Pat. No. 8,337,850, US 2018/0258177 or WO2018/156740. In other embodiments, the 4-1BB×TAA construct comprises a 4-1BB antigen-binding domain comprising a VH sequence that is at least about 80%, 85%, 90%, 95%, 96%, 97%, 98%, 99%, or 100% identical to the VH sequence of urelumab, utomilumab, or any one of the anti-4-1BB antibodies described in U.S. Pat. No. 8,337,850, US 2018/0258177 or WO2018/156740. The specific VH and VL sequences of MOR7480.1, one of the antibodies described in U.S. Pat. No. 8,337,850, are provided as SEQ ID NOs: 71 and 72, respectively, in Table 15. The CDRs of MOR7480.1 are provided in Table B below. The VH, VL and CDR sequences of the other 4-1BB antigen-binding domains described above can readily be determined by one of skill in the art with reference to the disclosures of U.S. Pat. No. 8,337,850, US 2018/0258177, WO2018/156740 WO2004/010947, WO2005/035584, US 2018/0258177, WO2017/077085, or WO2006126835.

TABLE B
MOR7480.1 CDRs

| | Defi- | | | |
|---|---|---|---|---|
| | nition | CDR1 | CDR2 | CDR3 |
| Heavy | Kabat | TYWIS | KIYPGDSYTNYSPSFQ | GYGIFDY |
| Chain | | (SEQ ID NO: | G | (SEQ ID |
| | | 358) | (SEQ ID NO: 359) | NO: 360) |
| | Chothia | GYSFSTY | YPGDSY | GYGIFDY |
| | | (SEQ ID NO: | (SEQ ID NO: 362) | (SEQ ID |
| | | 361) | | NO: 360) |
| | IMGT | GYSFSTYW | IYPGDSYT | ARGYGIFDY |
| | | (SEQ ID NO: | (SEQ ID NO: 364) | (SEQ ID |
| | | 363) | | NO: 365) |
| | AbM | GYSFSTYWIS | KIYPGDSYTN | GYGIFDY |
| | | (SEQ ID NO: | (SEQ ID NO: 367) | (SEQ ID |
| | | 366) | | NO: 360) |
| Light | Kabat | SGDNIGDQYAH | QDKNRPS | ATYTGFGSL |
| Chain | | (SEQ ID NO: | (SEQ ID NO: 369) | AV |
| | | 368) | | (SEQ ID |
| | | | | NO: 370) |
| | Chothia | SGDNIGDQYAH | QDKNRPS | ATYTGFGSL |
| | | (SEQ ID NO: | (SEQ ID NO: 369) | AV |
| | | 368) | | (SEQ ID |
| | | | | NO: 370) |
| | IMGT | NIGDQY | QDK | ATYTGFGSL |
| | | (SEQ ID NO: | | AV |
| | | 371) | | (SEQ ID |
| | | | | NO: 370) |
| | AbM | SGDNIGDQYAH | QDKNRPS | ATYTGFGSL |
| | | (SEQ ID NO: | (SEQ ID NO: 369) | AV |
| | | 368) | | (SEQ ID |
| | | | | NO: 370) |

Additional VH, VL and CDR sequences of antibodies that bind 4-1BB are described below and in Table 13; these antibodies are identified as 1B2, 1C3, I C8, 1G1, 2A7, 2E8, 2H9, 3D7, 3H1, 3E7, 3G4, 4B11, 4E6, 4F9, 4G10, 5E2, 5G8, and 6B3. In one embodiment, the 4-1BB×TAA antibody construct comprises a 4-1BB antigen-binding domain comprising a VH sequence and a VL sequence that is at least 85%, 90%, 95%, 96%, 97%, 98%, or 99% identical to the VH sequence and VL sequence of any one of antibodies 1B2, 1C3, 1C8, 1G1, 2A7, 2E8, 2H9, 3D7, 3H1, 3E7, 3G4, 4B11, 4E6, 4F9, 4F10, 5E2, 5G8, or 6B3 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 1B2 as set forth in Table 13 and a VL sequence that is at least 85%, 90%, 95%, 96%, 97%, 98%, or 99% identical to the VL sequence of antibody 1B2 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 1C3 as set forth in Table 13 and a VL sequence that is at least 85%, 90%. 95%, 96%, 97%, 98%, or 99% identical to the VL sequence of antibody 1C3 as set forth in Table 13, In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 1C8 as set forth in Table 13 and a VL sequence that is at least 85%, 90%, 95%, 96%, 97%, 98%, or 99% identical to the VL sequence of antibody 1C8 as set forth in Table 13. In one embodiment, the 4-1BB×TAA antibody construct comprises a 4-1BB antigen-binding domain comprising a VH sequence that is at least 85%, 90%, 95%, 96%, 97%, 98% or 99% identical to the VH sequence of antibody 1G1 as set forth in Table 13 and a VL sequence that is at least 85%, 90%, 95%, 96%, 97%, 98%, or 99% identical to the VL sequence of antibody 1G1 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 2A7 as set forth in Table 13 and a VL sequence that is at least 85%, 90%, 95%, 96%, 97%, 98%, or 99% identical to the VL sequence of antibody 2A7 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 2E8 as set forth in Table 13 and a VL sequence that is at least 85%, 90%, 95%, 96%, 97%, 98%, or 99% identical to the VL sequence of antibody 2E8 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 2H9 as set forth in Table 13 and a VL sequence that is at least 85%, 90%, 95%, 96%, 97%, 98%, or 99% identical to the VL sequence of antibody 2H9 as set forth in Table 13. in one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 3D7 as set forth in Table 13 and a VL sequence that is at least 85%, 90%, 95%, 96%, 97%, 98%, or 99% identical to the VL sequence of antibody 3D7 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 3H1 as set forth in Table13 and a VL sequence that is at least 85%, 90%, 95%, 96%, 97%, 98%, or 99% identical to the VL sequence of antibody 3H1 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 3E7 as set forth in Table 13 and a VL sequence that is at least 85%, 90%, 95%, 96%, 97%, 98%, or 99% identical to the VL sequence of antibody 3E7 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 3G4 as set forth in Table 13 and a VL sequence that is at least 85%, 90%, 95%, 96%, 97%, 98%, or 99% identical to the VL sequence of antibody 3G4 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 4B11 as set forth in Table 13 and a VL sequence that is at least 85%, 90%, 95%, 96%, 97%, 98%, or 99% identical to the VL sequence of antibody 4B11 as set forth in Table 13, In one embodiment, the 4-1BB×TAA antibody construct comprises a 4-1BB antigen-binding domain comprising a VH sequence that is at least 85%, 90%, 95%. 96%, 97%, 98%, or 99% identical to the VH sequence of antibody 4E6 as set forth in Table 13 and a VL sequence that is at least 85%, 90%, 95%, 96%, 97%, 98%, or 99% identical to the VL sequence of antibody 4E6 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 4F9 as set forth in Table 13 and a VL sequence that is at least 85%, 90%, 95%, 96%, 97%, 98%, or 99% identical to the VL sequence of antibody 4F9 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 4G10 as set forth in Table 13 and a VL sequence that is at least 85%, 90%, 95%, 96%, 97%, 98%, or 99% identical to the VL sequence of antibody 4G10 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 5E2 as set forth in Table 13 and a VL sequence that is at least 85%, 90%, 96%, 97%, 98%, or 99% identical to the VL sequence of antibody 5E2 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 5G8 as set forth in Table 13 and a VL, sequence that is at least 85%, 90%, 95%, 96%, 97%, 98%, or 99% identical to the VL sequence of antibody 5G8 as set forth in Table 13. In one embodiment, the 4-1BB×TAA antibody construct comprises a 4-1BB antigen-binding domain comprising a VH sequence that is at least 85%, 90%, 95%, 96%, 97%, 98%, or 99% identical to the VH sequence of antibody 6B3 as set forth in Table 13 and a VL sequence that is at least 85%, 90%, 95%, 96%, 97%, 98%, or 99% identical to the VL sequence of antibody 6B3 as set forth in Table 13.

In one embodiment, the 4-1BB×TAA antibody construct comprises the heavy chain CDRs and light chain CDRs of one of the antibodies listed in Table 13. The CDRs of these antibodies can be found in Table 18. In related embodiments, the 4-1BB×TAA antibody construct comprises the heavy chain CDRs and light chain CDRs of any one of antibodies 1C3, 1C8, 1G1, 2E8, 3E7, 4E6, 5G-8, or 6B3, described in Table 13.

In one embodiment, the 4-1BB×TAA antibody construct comprises a 4-1BB antigen-binding domain comprising a humanized VH sequence and a humanized VL sequence of any one of antibodies 1B2, 1C3, 1C8, 1G1, 2A7, 2E8, 2H9, 3D7, 3H1, 3E7, 3G4, 4B11, 4E6, 4F9, 4G10, 5E2, 5G8, or 6B3. Several exemplary humanized VH and VL sequences are described in Table 14 and have been used in the construction of several 4-1BB antibody constructs comprising humanized VH and VL sequences based on the mouse VH and VL sequences of anti-4-1BB antibodies 1C8, 1G1, and 5G8,

In one embodiment, the 4-1BB×TAA antibody construct comprises a 4-1BB antigen-binding domain comprising a humanized VH sequence and humanized VL sequence of antibody 1C8. In a related embodiment, the 4-1BB×TAA antibody construct comprises a VH sequence that is at least 85%, 90%, 95%, 96%, 97%, 98%, or 99% identical to the VH sequence of v28726, and a VL sequence that is at least 85%, 90%, 95%, 96%, 97%, 98%, or 99% identical to the VL sequence of variants 28726. In another embodiment, the 4-1BB×TAA antibody construct comprises a VH sequence that is at least 85%, 90%, 95%, 96%, 97%, 98%, or 99% identical to the VH sequence of v28727, and a VL sequence that is at least 85%, 90%, 95%, 96%, 97%, 98%, or 99% identical to the VL sequence of variant 28727. In another embodiment, the 4-1BB×TAA antibody construct comprises a VH sequence that is at least 85%, 90%, 95%,96%, 97%, 98%, or 99% identical to the VH sequence of v28728, and a VL sequence that is at least 85%, 90%, 95%, 96%, 97%, 98%, or 99% identical to the VL sequence of variant 28728. In another embodiment, the 4-1BB×TAA antibody construct comprises a VH sequence that is at least 85%, 90%, 95%, 96%, 97%, 98%, or 99% identical to the VH sequence of v28730, and a VL sequence that is at least 85%, 90%, 95%, 96%, 97%, 98%, or 99% identical to the VL sequence of variant 28730.

In one embodiment, the 4-lBB×TAA antibody construct comprises a 4-1BB antigen-binding domain comprising a humanized VH sequence and humanized VL sequence of antibody 1G1. In a related embodiment, the 4-1BB×TAA antibody construct comprises a VH sequence that is at least 85%, 90%, 95%, 96%, 97%, 98%, or 99% identical to the VH sequence of v28683, and a VL sequence that is at least 85%, 90%, 95%, 96%, 97%, 98%, or 99% identical to the VL sequence of variants 28683. In another embodiment, the 4-1BB×TAA antibody construct comprises a VH sequence that is at least 85%, 90%, 95%, 96%. 97%, 98%, or 99% identical to the VH sequence of v28684, and a VL sequence that is at least 85%. 90%, 95%, 96%, 97%, 98%, or 99% identical to the VL sequence of variant 28684. In another embodiment, the 4-1BB×TAA antibody construct comprises a VH sequence that is at least 85%, 90%, 95%, 96%, 97%, 98%, or 99% identical to the VH sequence of v28685, and a VL sequence that is at least 85%, 90%, 95%, 96%, 97%, 98%, or 99% identical to the VL sequence of variant 28685. In another embodiment, the 4-1BB×TAA antibody construct comprises a VH sequence that is at least 85%, 90%, 95%, 96%, 97%, 98%, or 99% identical to the VH sequence of v28686, and a VL sequence that is at least 85%, 90%, 95%, 96%. 97%, 98%, or 99% identical to the VL sequence of variant 28686. In another embodiment, the 4-1BB×TAA antibody construct comprises a VH sequence that is at least 85%, 90%, 95%, 96%, 97%, 98%, or 99% identical to the VH sequence of v28687, and a VL sequence that is at least 85%, 90%, 95%, 96%, 97%, 98%, or 99% identical to the VL sequence of variant 28687. In another embodiment, the 4-1BB×TAA antibody construct comprises a VH sequence that is at least 85%, 90%, 95%, 96%, 97%, 98%, or 99% identical to the VH sequence of v28688, and a VL sequence that is at least 85%, 90%, 95%, 96%, 97%, 98%, or 99% identical to the VL sequence of variant 28688. In another embodiment, the 4-1BB×TAA antibody construct comprises a VH sequence that is at least 85%, 90%, 95%, 96%, 97%, 98%, or 99% identical to the VH sequence of v28689, and a VL sequence that is at least 85%, 90%, 95%, 96%, 97%, 98%, or 99% identical to the VL sequence of variant 28689. In another embodiment, the 4-1BB×TAA antibody construct comprises a VH sequence that is at least 85%, 90%, 95%, 96%, 97%, 98%, or 99% identical to the VH sequence of v28690, and a VL sequence that is at least 85%, 90%, 95%, 96%, 97%, 98%, or 99% identical to the VL sequence of variant 28690. In another embodiment, the 4-1BB×TAA antibody construct comprises a VH sequence that is at least 85%, 90%, 95%, 96%, 97%, 98%, or 99% identical to the VH sequence of v28691, and a VL sequence that is at least 85%, 90%, 95%, 96%, 97%, 98%, or 99% identical to the VL sequence of variant 28691. In another embodiment, the 4-1BB×TAA antibody construct comprises a VH sequence that is at least 85%, 90%, 95%, 96%, 97%, 98%, or 99% identical to the VH sequence of v28692, and a VL sequence that is at least 85%, 90%, 95%, 96%, 97%, 98%, or 99% identical to the VL sequence of variant 28692. In another embodiment, the 4-1BB×TAA antibody construct comprises a VH sequence that is at least 85%, 90%, 95%, 96%, 97%, 98%, or 99% identical to the VH sequence of v28693, and a VL sequence that is at least 85%, 90%, 95%, 96%, 97%, 98%, or 99% identical to the VL sequence of variant 28693. In another embodiment, the 4-1BB×TAA antibody construct comprises a VH sequence that is at least 85%, 90% 95%, 96%, 97%, 98%, or 99% identical to the VH sequence of v28694, and a VL sequence that is at least 85%, 90%, 95%, 96%, 97%, 98%, or 99% identical to the VL sequence of variant 28694.

In one embodiment, the 4-1BB×TAA antibody construct comprises a 4-1BB antigen-binding domain comprising a humanized VH sequence and humanized VL sequence of antibody 5C8. In a related embodiment, the 4-1BB×TAA antibody construct comprises a VH sequence that is at least 85%, 90%, 95%, 96%, 97%, 98%, or 99% identical to the VH sequence of v28700, and a VL sequence that is at least 85%, 90%, 95%, 96%, 97%, 98%, or 99% identical to the VL sequence of variants 28700. In another embodiment, the 4-1BB×TAA antibody construct comprises a VH sequence that is at least 85%, 90%, 95%, 96%, 97%, 98%, or 99% identical to the VH sequence of v28704, and a VL sequence that is at least 85%, 90%, 95%, 96%, 97%, 98%, or 99% identical to the VL sequence of variant 28704. In another embodiment, the 4-1BB×TAA antibody construct comprises a VH sequence that is at least 85%, 90%, 95%, 96%, 97%, 98%, or 99% identical to the VH sequence of v28705, and a VL sequence that is at least 85%, 90%, 95%, 96%, 97%, 98%, or 99% identical to the VL, sequence of variant 28705, In another embodiment, the 4-1BB×TAA antibody construct comprises a VH sequence that is at least 85%, 90%, 95%, 96%, 97%, 98%, or 99% identical to the VH sequence of v28706, and a VL sequence that is at least 85%, 90%, 95%, 96%, 97%, 98%, or 99% identical to the VL sequence of 28706. In another embodiment, the 4-1BB×TAA antibody construct comprises a VH sequence that is at least 85%, 90%, 95%, 96%, 97%, 98%, or 99% identical to the VH sequence of v28711, and a VL sequence that is at least 85%, 90%, 95%, 96%, 97%, 98%, or 99% identical to the VL sequence of variant 28711. In another embodiment, the 4-1BB×TAA antibody construct comprises a VH sequence that is at least 85%, 90%, 95%, 96%, 97%, 98%, or 99% identical to the VH sequence of v28712 and a VL sequence that is at least 85%, 90%, 95%, 96%, 97%, 98%, or 99% identical to the VL sequence of variant 28712. In another embodiment, the 4-1BB×TAA antibody construct comprises a VH sequence that is at least 85%, 90%, 95%, 96%, 97%, 98%, or 99% identical to the VH sequence of v28713, and a VL sequence that is at least 85%, 90%, 95%, 96%, 97%, 98%, or 99% identical to the VL sequence of variant 28713. In another embodiment, the 4-1BB×TAA antibody construct comprises a VH sequence that is at least 85%, 90%, 95%, 96%, 97%, 98%, or 99% identical to the VH sequence of v28696, and a VL sequence that is at least 85%, 90%, 95%, 96%, 97%, 98%, or 99% identical to the VL sequence of variant 28696. In another embodiment, the 4-1BB×TAA antibody construct comprises a VH sequence that is at least 85%, 90%, 95%, 96%, 97%, 98%, or 99% identical to the VH sequence of v28697, and a VL sequence that is at least 85%, 90%, 95%, 96%, 97%, 98%, or 99% identical to the VL sequence of variant 28697. In another embodiment, the 4-1BB×TAA antibody construct comprises a VH sequence that is at least 85%, 90%, 95%, 96%, 97%, 98%, or 99% identical to the VH sequence of v28698, and a VL sequence that is at least 85%, 90%, 95%, 96%, 97%, 98%, or 99% identical to the VL sequence of variant 28698. In another embodiment, the 4-1BB×TAA antibody construct comprises a VH sequence that is at least 85%, 90%, 95%, 96%, 97%, 98%, or 99% identical to the VH sequence of v28701, and a VL sequence that is at least 85%, 90%, 95%, 96%, 97%, 98%, or 99% identical to the VL sequence of variant 28701. In another embodiment, the 4-1BB×TAA antibody construct comprises a VH sequence that is at least 85%, 90%, 95%, 96%, 97%, 98%, or 99% identical to the VH sequence of v28702, and a VL sequence that is at least 85%, 90%, 95%, 96%, 97%, 98%, or 99% identical to the VL sequence of variant 28702, In another embodiment, the 4-1BB×TAA antibody construct comprises a VH sequence that is at least 85%, 90%, 95%, 96%, 97%, 98%, or 99% identical to the VH sequence of v28703, and a VL sequence that is at least 85%, 90%, 95%, 96%, 97%, 98%, or 99% identical to the VL sequence of variant 28703. In another embodiment, the 4-1BB×TAA antibody construct comprises a VH sequence that is at least 85%, 90%, 95%, 96%, 97%, 98%, or 99% identical to the VH sequence of v28707, and a VL sequence that is at least 85%, 90%, 95%, 96%, 97%, 98%, or 99% identical to the VL sequence of variant 28707.

In one embodiment, the 4-1BB×TAA antibody construct comprises the heavy chain CDRs and light chain CDRs of any one of the humanized antibodies v28726, v28727, v28728, v28730, v28700, v28704, v28705, v28706, v28711, v28712, v28713, v28696, v28697, v28698, v28701, v28702, v28703, v28707, v28683, v28684, v28685, v28686, v28687, v28688, v28689, v28690, v28691, v28692, v28694, and v28695. The CDRs of these antibodies can be found in Table 18.

In other embodiments, the 4-1BB×TAA antibody construct comprises a 4-1BB antigen-binding domain, and a TAA antigen-binding domain, wherein the 4-1BB-binding domain and the TAA antigen-binding domain are linked directly or indirectly to a scaffold, and the 4-1BB antigen-binding domain comprises one, two, or three heavy chain CDRs and/or one, two, or three light chain CDRs of v28726, v28727, v28728, v28730, v20022, v28700, v28704, v28705, v28706, v28711, v28712, v28713, v20036, v28696, v28697, v28698, v28701, v28702, v28703, v28707, v20023, v28683, v28684, v28685, v28686, v28687, v28688, v28689, v28690, v28691, v28692, v28694, and v28695.

In other embodiments, the 4-1BB×TAA antibody construct comprises a 4-1BB-binding domain that is capable of binding to an ECD of human 4-1BB and is cyno cross-reactive. The term “cyno cross-reactive” as used herein is meant to describe binding domains that bind to a target from one species (for example, human or mouse) and are able also to bind to the same target expressed in a cynomolgus monkey. In some embodiments, the antibody construct comprises a 4-1BB-binding domain that can bind to an ECD of mouse 4-1BB.

### TAAs and TAA Antigen-Binding Domains

The 4-1BB×TAA antibody constructs described herein comprise a 4-1BB-binding domain that binds to a 4-1BB extracellular domain (4-1BB ECD), and a tumor-associated antigen (TAA) antigen binding domain that binds to a TAA, wherein the 4-1BB-binding domain and the TAA antigen binding domain are linked directly or indirectly to a scaffold. In some embodiments, the 4-1BB×TAA antibody construct comprises a first TAA antigen-binding domain and a second TAA antigen-binding domain linked directly or indirectly to a scaffold.

As used herein “tumor-associated antigen” or “TAA” refers to an antigen that is expressed by cancer cells. A tumor-associated antigen may or may not be expressed by normal cells (non-tumor cells). When a TAA is not expressed by normal cells (i.e. when it is unique to tumor cells) it may also be referred to as a “tumor-specific antigen.” When a TAA is not unique to a tumor cell, it is also expressed on a normal cell under conditions that fail to induce a state of immunologic tolerance to the antigen. The expression of the antigen on the tumor may occur under conditions that enable the immune system to respond to the antigen. TAAs may be antigens that are normally present at low levels on normal cells but which are expressed at higher levels on tumor cells. Those TAAs of greatest clinical interest are differentially expressed compared to the corresponding normal tissue and allow for a preferential recognition of tumor cells by specific T-cells or immunoglobulins. In some embodiments, TAAs may be membrane-bound antigens, or antigens that are localized on the surface of a tumor cell.

In one embodiment, the 4-1BB×TAA antibody construct comprises a TAA antigen-binding domain that binds to a TAA that is expressed at high levels in tumor cells. For example, the tumor cells may express the TAA at greater than about 1 million copies per cell. In another embodiment, the 4-1BB×TAA antibody construct comprises at least one TAA antigen-binding domain that binds to a TAA that is expressed at medium levels in tumor cells. For example, the tumor cells may express the TAA at greater than about 100,000 to about 1 million copies per cell. In one embodiment, the 4-1BB×TAA antibody construct comprises at least one TAA antigen-binding domain that binds to a TAA that is expressed at low levels in tumor cells. For example, the tumor cells may express the TAA at less than about 100,000 copies per cell. In one embodiment, the 4-1BB×TAA antibody construct binds to a TAA that is expressed at higher levels on tumor cells than on normal cells.

In some embodiments the 4-1BB×TAA antibody construct binds to a TAA that is expressed on a breast cancer cell, a lung cancer cell, an ovarian cancer cell, a colon cancer cell, a skin cancer cell, a bladder cancer cell, a lymphoma or leukemic cell, a kidney cancer cell, a pancreatic cancer cell, a stomach cancer cell, an oesophageal cancer cell, a prostate cancer cell, a thyroid cancer cell or other non-liver cancer cell.

The 4-1BB×TAA antibody construct may comprise varying numbers of TAA antigen-binding domains. Accordingly, in certain embodiments, the 4-1BB×TAA antibody construct comprises a 4-1BB-binding domain and a TAA antigen-binding domain, wherein the 4-1BB-binding domains and the TAA antigen-binding domains are linked directly or indirectly to a scaffold. In other embodiments, the 4-1BB×TAA antibody construct comprises two 4-1BB-binding domains and one TAA antigen-binding domain, wherein the 4-1BB-binding domains and the TAA antigen-binding domains are linked directly or indirectly to a scaffold. In still other embodiments, the 4-1BB×TAA antibody construct comprises one or more 4-1BB-binding domains, and two TAA antigen-binding domains, wherein the 4-1BB-binding domains and the TAA antigen-binding domains are linked directly or indirectly to a scaffold. In related embodiments, where the antibody construct comprises two or more TAA antigen-binding domains, each TAA antigen-binding domain may bind to the same epitope of one TAA, or to different epitopes of the same TAA, or to different TAAs.

The TAA antigen-binding domains may be in scFv, Fab or sdAb formats. Thus, in one embodiment, the TAA antigen-binding domain of the 4-1BB×TAA antibody construct is in a Fab format. In alternate embodiments, the TAA antigen-binding domain is in a scFv format. In additional embodiments, the 4-1BB×TAA antibody construct comprises more than one TAA antigen-binding domain, wherein at least one TAA antigen-binding domain is in an scFv format. In other embodiments where the 4-1BB×TAA comprises more than one TAA antigen-binding domain, at least two of the antigen-binding domains are in the scFv format.

In one embodiment, the 4-1BB×TAA antibody construct comprises a TAA antigen-binding domain that binds to a TAA selected from, but not limited to, carbonic anhydrase IX, alpha-fetoprotein (AFP), alpha-actinin-4, A3, antigen specific for A33 antibody, ALK (anaplastic lymphoma receptor tyrosine kinase), ART-4, B7, B7, B7-H4, Ba 733, BAGE, BCMA, BrE3-antigen, CA125, CAMEL, CAP-1, CASP-8/m, CCL19, CCL21, CD1, CD1a, CD2, CD3, CD4, CD5, CD8, CD11A, CD14, CD15, CD16, CD18, CD19, CD20, CD21, CD22, Cd23. CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CSF1R, CTLA-4, CXCR4, CXCR7, CXCL12, HIF-1a, colon-specific antigen-p (CSAp), CEA, CEACAMS, CEACAM6, c-Met, DAM, DL3, EGFR, EGFRvIII, EGP-1 (TROP-2), EGP-2, ELF2-M, Ep-CAM, EphA2, fibroblast growth factor (FGF), Flt-1, Flt-3, folate receptor, G250 antigen, GAGE, GD2, gp100, GPC3, GRO-13, HLA-DR, HM1.24, human chorionic gonadotropin (HCG) and its subunits, HER2/neu, HMGB-1, hypoxia inducible factor (HIF-I), HSP70-2M, HST-2, Ia. IGF-1R, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR-/FUT macrophage migration inhibitory factor (MIF), MAGE, MAGE-3, MART-1, MART-2, mCRP, MCP-1, melanoma glycoprotein, mesothelin, MIP-1A, MIP-1B, MIF, MUC1, MUC2, MUC3, MUC4, MUC5ac, MUC 13, MUC16, MUM-1/2, MUM-3, NaPi2B, NCA66, NCA95, NCA90, NY-ESO-1, PAM4 antigen, pancreatic cancer mucin, PD-1, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, ROR1, T101, SAGE, 5100. survivin, survivin-2B, TAC, TAG-72, tenascin, TRAG-3, TRAIL receptors, TGFβ, TNF-alpha, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207).

In one embodiment, the 4-1BB×TAA antibody constructs described herein comprise a 4-1BB-binding, domain that binds to a 4-1BB extracellular domain (4-1BB ECD), and a TAA antigen-binding domain that binds to folate receptor (FRα), wherein the first 4-1BB-binding domain and the TAA antigen binding domain are linked directly or indirectly to a scaffold. In one embodiment, the 4-1BB×TAA antibody constructs described herein comprise a 4-1BB-binding domain that binds to a 4-1BB extracellular domain (4-1BB ECD), and a tumor-associated antigen (TAA)-antigen binding domain that binds to Solute Carrier Family 34 Member 2 (SLC34A2, NaPi2b), wherein the first 4-1BB-binding domain and the TAA antigen binding domain are linked directly or indirectly to a scaffold. In one embodiment, the 4-1BB×TAA antibody constructs described herein comprise a 4-1BB-binding domain that binds to a 4-1BB extracellular domain (4-1BB ECD), and a tumor-associated antigen (TAA)-antigen binding domain that binds to HER2, wherein the first 4-1BB-binding domain and the TAA antigen binding domain are linked directly or indirectly to a scaffold. In one embodiment, the 4-1BB×TAA antibody constructs described herein comprise a 4-1BB-binding domain that binds to a 4-1BB extracellular domain (4-1BB ECD), and a tumor-associated antigen (TAA)-antigen binding domain that binds to mesothelin, wherein the first 4-1BB-binding domain and the TAA antigen binding domain are linked directly or indirectly to a scaffold. In one embodiment, the 4-1BB×TAA antibody constructs described herein comprise a 4-1BB-binding domain that hinds to a 4-1BB extracellular domain (4-1BB ECD), and a tumor-associated antigen (TAA)-antigen binding domain that binds to Solute Carrier Family 39 Member 6 (SLC3A6, LIV-1), wherein the first 4-1BB-binding domain and the TAA antigen binding domain are linked directly or indirectly to a scaffold.

The TAA antigen-binding domains may be constructed from the sequences of known antibodies directed against TAAs. Many such antibodies are known in the art and may be commercially obtained from a number of sources. For example, a variety of antibody secreting hybridoma lines are available from the American Type Culture Collection (ATCC, Manassas, Va.) In addition, a number of antibodies against various TAAs have been deposited at the ATCC and/or have published variable domain sequences and may be used to prepare the TAA antigen-binding domains of the antibody constructs. The skilled artisan will appreciate that antibody sequences or antibody-secreting hybridomas against various TAAs may be obtained by a simple search of the ATCC, NCBI, and/or USPTO databases. Alternatively, antibodies that specifically bind to a desired TAA may be generated according to methods known in the art and described elsewhere herein.

### FRα Antigen-Binding Domains

In one embodiment, the 4-1BB×TAA antibody construct is a 4-1BB×FRα antibody construct comprising a 4-1.BB antigen-binding domain and a FRα antigen-binding domain wherein the 4-1BB binding domain and the Frα antigen-binding domain are linked directly or indirectly to a scaffold.

FRα is a member of the folate receptor family which functions to bind folic acid and transports 5-methyltetrahydrofolate into cells. FRα is also known as folate receptor 1, FOLR, FOLR1, FBP or MOv18 and is expressed in normal cells as well as tumor cells as a secreted protein that exists in soluble form or is anchored to the membrane of cells through a glycosyl-phosphatidylinositol (GPI) linkage. FRα is further described in Cheung et al. (2016) Oncotarget 7:52553-52574. The polypeptide sequence of this protein is described in GenBank Accession No, AAB05827.1 and UniProt P15328, and provided here as SEQ ID NO:80.

FRα antigen-binding domains may be derived from anti-FRα antibodies known in the art, including but not limited to: farletuzumab (Morphotek, described in WO2004/003388 and WO2005/080431), mirvetuximab (ImmunoGen, described in WO2011106528). Other anti-FRα antibodies are described in U.S. Pat. No. 8,388,972 (Advanced Accelerator Applications), WO2018/098277 (Eisai R&D Management Co.), U.S. Pat. No. 9,695,237 (Kyowa Hakko Kirin Co.), WO2015/196167 (Bioalliance), WO2016/079076 (Roche), and WO2018/071597 (Sutro), 1001321 In one embodiment, the 4-1BB×TAA antibody construct comprises a FRα antigen-binding domain that can compete with farletuzumab for binding to an epitope of FRα. In another embodiment, the 4-1BB×TAA antibody construct comprises a FRα antigen-binding domain that can compete with mirvetuximab for binding to an epitope of Rα. In still other embodiments, the 4-1BB×TAA antibody construct comprises a FRα antigen-binding domain that can compete for binding to an epitope of FRα with any one of the anti-FRα antibodies described in U.S. Pat. No. 8,388,972, WO2018/098277, U.S. Pat. No. 9,695,237, WO2015/196167, WO2016/079076, or WO2018/071597.

In other embodiments, the 4-1BB×TAA antibody construct comprises a FRα antigen-binding domain that binds to the same epitope of FRα as farletuzumab or mirvetuximab, or any one of the FRα antibodies described in U.S. Pat. No. 8,388,972, WO2018/098277, U.S. Pat. No. 9,695,237, WO2015/196167, WO2016/079076, or WO2018/071597.

In one embodiment, the 4-1BB×TAA antibody construct comprises an FRα antigen-binding domain that comprises at least one, two, or all three heavy chain CDRs and/or at least one, two, or all three light chain CDRs of farletuzumab, mirvetuximab, or any one of the anti-FRα antibodies described in U.S. Pat. No. 8,388,972, WO2018/098277, U.S. Pat. No. 9,695,237, WO2.015/196167, WO2016/079076, or WO2018/071597. In an alternate embodiment, the 4-1BB×TAA antibody construct comprises a FRα antigen-binding domain that comprises at least one, two, or all three heavy chain CDRs and at least one, two, or all three light chain CDRs of farletuzumab, mirvetuximab, or any one of the anti-FRα antibodies described in U.S. Pat. No. 8,388,972, WO2018/098277, U.S. Pat. No. 9,695,237, WO2015/196167, WO2016/079076, or WO2018/071597, In other embodiments, the 4-1BB×TAA construct comprises a FRα antigen-binding domain comprising a VH sequence that is at least about 80%, 85%, 90%, 95%, 96%, 97%, 98%, 99%, or 100% identical to the VH sequence of farletuzumab, mirvetuximab, or any one of the anti-FRα antibodies described in U.S. Pat. No. 8,388,972, WO2018/098277, U.S. Pat. No. 9,695,237, WO2015/196167, WO2016/079076, or WO2018/071597. The specific sequences of the CDRs for mirvetuximab and farletuzumab are described in Table C and the VH and VL sequences of these antibodies are provided in Table 17; the others can readily be determined by one of skill in the art with reference to the disclosures of U.S. Pat. No. 8,388,972, WO2018/098277, U.S. Pat. No. 9,695,237, WO2015/196167, WO2016/079076, or WO2018/071597.

TABLE C
CDR sequences of exemplary anti-FRα antibodies

| | | | |
|---|---|---|---|
| Defi- | | | |
| nition | CDR1 | CDR2 | CDR3 |

Mirvetuximab

| | | | | |
|---|---|---|---|---|
| Heavy | Kabat | GYFMN | RIHPYDGDTFY | YDGSRAMDY |
| chain | | (SEQ ID | NQKFQG (SEQ | (SEQ ID NO: |
| | | NO: 372) | ID NO: 373) | 374) |
| | Chothia | GYTFTGY | HPYDGD | YDGSRAMDY |
| | | (SEQ ID | (SEQ ID NO: | (SEQ ID NO: |
| | | NO: 375) | 376) | 374) |
| | IMGT | GYTFTGYF | IHPYDGDT | TRYDGSRAMDY |
| | | (SEQ ID | (SEQ ID NO: | (SEQ ID NO: |
| | | NO: 377) | 378) | 379) |
| | AbM | GYTFTGYFMN | RIHPYDGDTF | YDGSRAMDY |
| | | (SEQ ID | (SEQ ID NO: | (SEQ ID NO: |
| | | NO: 380) | 381) | 374) |
| Light | Kabat | KASQSVSFAG | RASNLEA | QQSREYPYT |
| chain | | TSLMH (SEQ | (SEQ ID NO: | (SEQ ID NO: |
| | | ID NO: 382) | 383) | 384) |
| | Chothia | KASQSVSFAG | RASNLEA | QQSREYPYT |
| | | TSLMH (SEQ | (SEQ ID NO: | (SEQ ID NO: |
| | | ID NO: 382) | 383) | 384) |
| | IMGT | QSVSFAGTSL | RAS | QQSREYPYT |
| | | (SEQ ID NO: | (SEQ ID NO: | |
| | | 385) | 384) | |
| | AbM | KASQSVSFAG | RASNLEA | QQSREYPYT |
| | | TSLMH (SEQ | (SEQ ID NO: | (SEQ ID NO: |
| | | ID NO: 382) | 383) | 384) |

Farletuzumab

| | | | | |
|---|---|---|---|---|
| Heavy | Kabat | GYGLS | MISSGGSYTYY ADSVKG | HGDDPAWFAY |
| chain | | (SEQ ID | (SEQ ID NO: | (SEQ ID NO: |
| | | NO: 386) | 387) | 388) |
| | Chothia | GFTFSGY | SSGGSY | HGDDPAWFAY |
| | | (SEQ ID | (SEQ ID NO: | (SEQ ID NO: |
| | | NO: 389) | 390) | 388) |
| | IMGT | GFTFSGYG | ISSGGSYT | ARHGDDPAWFAY |
| | | (SEQ ID | (SEQ ID NO: | (SEQ ID NO: |
| | | NO: 391) | 392) | 393) |
| | AbM | GFTFSGYGLS | MISSGGSYTY | HGDDPAWFAY |
| | | (SEQ ID | (SEQ ID NO: | (SEQ ID NO: |
| | | NO: 394) | 395) | 388) |
| Light | Kabat | SVSSSISSNNLH | GTSNLAS | QQWSSYPYMYT |
| chain | | (SEQ ID | (SEQ ID NO: | (SEQ ID NO: |
| | | NO: 396) | 397) | 398) |
| | Chothia | SVSSSISSNNLH | GTSNLAS | QQWSSYPYMYT |
| | | (SEQ ID | (SEQ ID NO: | (SEQ ID NO: |
| | | NO: 396) | 397) | 398) |
| | IMGT | SSISSNN | QQWSSYPYMYT | |
| | | (SEQ ID | GTS | (SEQ ID NO: |
| | | NO: 399) | | 398) |
| | AbM | SVSSSISSNNLH | GTSNLAS | QQWSSYPYMYT |
| | | (SEQ ID NO: | (SEQ ID NO: | (SEQ ID NO: |
| | | 396) | 397) | 398) |

Alternatively, FRα antigen-binding domains may be derived from novel antibodies that are generated according to methods known in the art.

Additional anti-FRα antibody VH and VL sequences are provided in Table 17. In one embodiment, the 4-1BB×FRα antibody construct comprises a FRα antigen-binding domain having a sequence that is at least 85%, 90%, 95%, 96%. 97%, 98%, or 99% identical to the VH sequence of antibody 8K22 or 1H06 and a VL sequence that is at least 85%, 90%, 95%, 96%, 97%. 98%, or 99% identical to the VL sequence of antibody 8K22 or 1H06. The CDRs of these antibodies are provided in Table 18. In one embodiment, the 4-1BB×TAA antibody construct comprises the heavy chain CDRs and light chain CDRs of antibody 8K22 or 1H06.

In one embodiment, the 4-1BB×FRα antibody construct comprises a 4-1BB antigen-binding domain comprising the CDRs of any one of the humanized antibodies v28726, v28727, y28728, v28730, v28700, y28704, v28705, v28706, y28711, v28712, v28713, v28696, y28697, v28698, v28701, y28702, v28703, v28707, y28683, v28684, v28685, v28686, v28687, v28688, v28689, v28690, v28691, v28692, v28694, and v28695 and a FRα antigen-binding domain linked to scaffold. In one embodiment, the 4-1BB×antibody construct comprises a 4-1BB antigen-binding domain comprising the CDRs of any one of the humanized antibodies v28726, v28727, v28728, v28730, v28700, v28704, v28705, v28706, v28711, v28712, v28713, v28696, v28697, v28698, v28701, v28702, v28703, v28707, v28683, v28684, v28685, v28686, v28687, v28688, v28689, v28690, v28691, v28692, v28694, and v28695 and a FRα antigen-binding domain comprising the CDRs of 8K22 or 1H06.

### SLC34A2/NaPi2b Antigen-Binding Domains

In one embodiment, the 4-1BB×TAA antibody construct is a 4-1BB×NaPi2b antibody construct comprising a 4-1BB antigen-binding domain and a NaPi2b antigen-binding domain wherein the 4-1BB binding domain and the NaPi2b antigen-binding domain are linked directly or indirectly to a scaffold.

SLC34A2 is a pH-sensitive sodium-dependent phosphate transporter. Also known as NaPi2b as well as NAPI-3B, NAPI-IIb, NPTIIb, this protein is expressed in some normal epithelial cells in the lung, gut and mammary gland and has a function in transporting phosphate ions. NaPi2b is found highly expressed on tumor cells, primarily in lung and ovarian cancers (Lin K et al, Clin Cancer Res. 2015 Nov. 15; 21(22):5139-50). NaPi2b is a multispan membrane protein, with extracellular domains of 14, 129, 57 and 6 amino acids. The polypeptide sequence of this protein is described in NGBI Reference Sequence: NP_001171470.1 and UniProt O95436, and provided herein as SEQ ID NO:81

NaPi2b antigen-binding domains may be derived from antibodies known in the art, including but not limited to: lifastuzumab (Genentech, Seattle Genetics, described in WO2011/066503), MX-35 (Ludwig Institute, described in WO2009/097128), and the antibodies described by Mersana Therapeutics in US2017/0266311. Alternatively, NaPi2b antigen-binding domains may be derived from novel antibodies that are generated according to methods known in the art and described elsewhere herein.

In one embodiment, the 4-1BB×TAA antibody construct comprises an NaPi2b antigen-binding domain that can compete with lifastuzumab for binding to an epitope of NaPi2b. In another embodiment, the 4-1BB×TAA antibody construct comprises an NaPi2b antigen-binding domain that can compete with MX-35 for binding to an epitope of NaPi2b. In still other embodiments, the 4-1BB×TAA antibody construct comprises an NaPi2b antigen-binding domain that can compete for binding to an epitope of NaPi2b with any one of the anti-NaPi2b antibodies described in WO2011/066503, WO2009/097128, or US2017/0266311.

In other embodiments, the 4-lBB×TAA antibody construct comprises an NaPi2b antigen-binding domain that binds to the same epitope of NaPi2b as lifastuzumab or MX-35, or any one of the anti-NaPi2b antibodies described in WO2011/066503, WO2009/097128, or US2017/0266311.

In one embodiment, the 4-1BB×TAA antibody construct comprises an NaPi2b antigen-binding domain that comprises at least one, two, or all three heavy chain CDRs and/or at least one, two, or all three light chain CDRs of lifastuzumab or MX-35, or any one of the anti-NaPi2b antibodies described in WO2011/066503, WO2009/097128, or US2017/0266311. In an alternate embodiment, the 4-1BB×TAA antibody construct comprises an NaPi2b antigen-binding domain that comprises at least one, two, or all three heavy chain CDRs and at least one, two, or all three light chain CDRs of lifastuzumab or MX-35, or any one of the anti-NaPi2b antibodies described in WO2011/066503, WO2009/097128, or US2017/026631 In other embodiments, the 4-1BB×TAA construct comprises an NaPi2b antigen-binding domain comprising a VH sequence that is at least about 80%, 85%, 90%, 95%, 96%, 97%, 98%, 99%, or 100% identical to the VH sequence of lifastuzumab or MX-35, or any one of the anti-NaPi2b antibodies described in WO2011/066503, WO2009/097128, or US2017/0266311. The specific sequences of the CDRs of exemplary anti-NaPi2b antibodies are described in Table D; the VH and VL sequences of these antibodies are found in Table 17. Other anti-NaPi2b antibody sequences can readily be determined by one of skill in the art with reference to the disclosures of WO2011/066503, WO2009/097128, or US2017/0266311.

TABLE D
CDR sequences of exemplary anti-NaPi2b antibodies

| | | | |
|---|---|---|---|
| Defi- | | | |
| nition | CDR1 | CDR2 | CDR3 |

Lifastuzumab

| | | | | |
|---|---|---|---|---|
| Heavy | Kabat | DFAMS | TIGRVAFHTY | HRGFDVGHFDF |
| chain | | (SEQ ID | YPDSMKG (SEQ | (SEQ ID NO: |
| | | NO: 400) | ID NO: 401) | 402) |
| | Chothia | GFSFSDF | GRVAFH | HRGFDVGHFDF |
| | | (SEQ ID | (SEQ ID NO: | (SEQ ID NO: |
| | | NO: 403) | 404) | 402) |
| | IMGT | GFSFSDFA | IGRVAFHT | ARHRGFDVGHFDF |
| | | (SEQ ID | (SEQ ID NO: | (SEQ ID NO: |
| | | NO: 405) | 406) | 407) |
| | AbM | GFSFSDFAMS | TIGRVAFHTY | HRGFDVGHFDF |
| | | (SEQ ID | (SEQ ID NO: | (SEQ ID NO: |
| | | NO: 408) | 409) | 402) |
| Light | Kabat | RSSETLVHSS | RVSNRFS | FQGSFNPLT |
| chain | | GNTYLE | (SEQ ID NO: | (SEQ ID NO: |
| | | (SEQ ID | 411) | 412) |
| | | NO: 410) | | |
| | Chothia | RSSETLVHSS | RVSNRFS | FQGSFNPLT |
| | | GNTYLE | (SEQ ID NO: | (SEQ ID NO: |
| | | (SEQ ID | 411) | 412) |
| | | NO: 410) | | |
| | IMGT | ETLVHSSGN | RVS | FQGSFNPLT |
| | | TY (SEQ ID | | (SEQ ID NO: |
| | | NO: 468) | | 412) |
| | AbM | RSSETLVHSS | RVSNRFS | FQGSFNPLT |
| | | GNTYLE | (SEQ ID NO: | (SEQ ID NO: |
| | | (SEQ ID | 411) | 412) |
| | | NO: 410) | | |

MX-35

| | | | | |
|---|---|---|---|---|
| Heavy | Kabat | GYNIH | AIYPGNGDTS | GETARATFAY |
| chain | | (SEQ ID | YKQKFRG (SEQ | (SEQ ID NO: |
| | | NO: 413) | ID NO: 414) | 415) |
| | Chothia | GYTFTGY | YPGNGD | GETARATFAY |
| | | (SEQ ID | (SEQ ID NO: | (SEQ ID NO: |
| | | NO: 416) | 417) | 415) |
| | IMGT | GYTFTGYN | IYPGNGDT | ARGETARATFAY |
| | | (SEQ ID | (SEQ ID NO : | (SEQ ID NO: |
| | | NO: 418) | 419) | 420) |
| | AbM | GYTFTGYNIH | AIYPGNGDTS | GETARATFAY |
| | | (SEQ ID | (SEQ ID NO: | (SEQ ID NO: |
| | | NO: 421) | 422) | 415) |
| Light | Kabat | SASQDIGNF | YTSSLYS | QQYSKLPLT |
| chain | | LN (SEQ ID | (SEQ ID NO: | (SEQ ID NO: |
| | | NO: 423) | 424) | 425) |
| | Chothia | SASQDIGNF | YTSSLYS | QQYSKLPLT |
| | | LN (SEQ ID | (SEQ ID NO: | (SEQ ID NO: |
| | | NO: 423) | 424) | 425) |
| | IMGT | QDIGNF | YTS | QQYSKLPLT |
| | | (SEQ ID | | (SEQ ID NO: |
| | | NO: 469) | | 425) |
| | AbM | SASQDIGNF | YTSSLYS | QQYSKLPLT |
| | | LN (SEQ | (SEQ ID NO: | (SEQ ID NO: |
| | | ID NO: | 424) | 425) |
| | | 423) | | |

Alternatively, NaPi2b antigen-binding domains may be derived from novel antibodies that are generated according to methods known in the art.

### HER2 Antigen-Binding Domains

In one embodiment, the 4-1BB×TAA antibody construct is a 4-1BB×HER2 antibody construct comprising a 4-1BB antigen-binding domain and a HER2 antigen-binding domain wherein the 4-1BB binding domain and the RER2 antigen-binding domain are linked directly or indirectly to a scaffold.

HER2 (also known as ErbB2) is a receptor protein tyrosine kinase which belongs to the human epidermal growth factor receptor (HER) family which includes EGFR, HER2, HER3 and HER4 receptors. The extracellular (ecto) domain of HER2 comprises four domains, Domain I (ECD1, amino acid residues from about 1-195), Domain II (ECD2, amino acid residues from about 196-319), Domain III (ECD3, amino acid residues from about 320-488), and Domain IV (ECD4, amino acid residues from about 489-630) (residue numbering without signal peptide). See Garrett et al. Mol. Cell. 11: 495-505 (2003), Cho et al. Nature 421: 756-760 (2003), Franklin et al. Cancer Cell 5:317-328 (2004). Tse et al. Cancer Treat Rev. 2012 April; 38(2):133-42 (2012), or Proc. Natl. Acad. Sci. 90:1746-1750 (1993). The polypeptide sequence of HER2 is described in UniProt P04626 and included herein as SEQ ID NO:82.

HER2 antigen-binding domains may be derived from antibodies known in the art, including but not limited to: trastuzumab (Genentech, described for example in U.S. Pat. Nos. 5,821.337. and 6,528,624), or pertuzurnab (Genentech, U.S. Pat. No. 7,862,217). The online Therapeutic Antibodies Database (Tabs, hosted by Craic Computing LLC, tabs.craic.com) identifies many additional anti-HER2 antibodies that provide suitable sequences for preparing; the anti-HER2 antigen-binding domains of the 4-1BB×TAA antibody construct.

In one embodiment, the 4-1BB×TAA antibody construct comprises a HER2 antigen-binding domain that can compete with trastuzumab for binding to an epitope of HER2. In another embodiment, the 4-1BB×TAA antibody construct comprises a HER2 antigen-binding domain that can compete with pertuzumab for binding to an epitope of HER2. In still other embodiments, the 4-1BB×TAA antibody construct comprises a HER2 antigen-binding domain that can compete for binding to an epitope of HER2 with any one of the anti-HER2 antibodies described in U.S. Pat. Nos. 5,821,337, 6,528,624, or 7,862,217.

In other embodiments, the 4-1BB×TAA antibody construct comprises a HER2 antigen-binding domain that binds to the same epitope of HER2 as trastuzumab or pertuzumab, or any one of the anti-HER2 antibodies described in U.S. Pat. Nos. 5,821,337, 6,528,624, or 7,862,217.

In one embodiment, the 4-1BB×TAA antibody construct comprises a HER2 antigen-binding domain that comprises at least one, two, or all three heavy chain CDRs and/or at least one, two, or all three light chain CDRs of trastuzumab or pertuzumab or margetuximab, or any one of the anti-HER2 antibodies described in U.S. Pat. Nos. 5,821,337, 6,528,624, or 7,862,217. In an alternate embodiment, the 4-1BB×TAA antibody construct comprises HER2 antigen-binding domain that comprises at least one, two, or all three heavy chain CDRs and at least one, two, or all three light chain CDRs of trastuzumab or pertuzumab or margetuximab, or any one of the anti-HER2 antibodies described in U.S. Pat. Nos. 5,821,337, 6,528,624, or 7,862.217. 1n other embodiments, the 4-1BB×TAA construct comprises a HER2 antigen-binding domain comprising a VH sequence that is at least about 80%, 85%, 90%, 95%, 96%, 97%, 98%, 99%, or 100% identical to the VH sequence of trastuzumab or pertuzumab or margetuximab, or any one of the anti-HER2 antibodies described in U.S. Pat. Nos. 5,821,337, 6,528,624, or 7,862,217. The specific sequences of the CDRs of exemplary anti HER2 antibodies are described in Table E; the VH and VL sequences of these antibodies are found in Table 17. Other anti-HER2 antibody sequences can readily be determined by one of skill in the art with reference at least to the disclosures of WO2011/066503, WO2009/097128, or US2017/0266311.

TABLE E
CDR sequences of exemplary anti-HER2 antibodies

| | | | |
|---|---|---|---|
| Defi- | | | |
| nition | CDR1 | CDR2 | CDR3 |

trastuzumab

| | | | | |
|---|---|---|---|---|
| Heavy | Kabat | DTYIH | RIYPTNGYTR | WGGDGFYAMDY |
| chain | | (SEQ ID | YADSVKG (SEQ | (SEQ ID NO: |
| | | NO: 426) | ID NO: 427) | 428) |
| | Chothia | GFNIKDT | YPTNGY | WGGDGFYAMDY |
| | | (SEQ ID | (SEQ ID NO: | (SEQ ID NO: |
| | | NO: 429) | 430) | 428) |
| | IMGT | GFNIKDTY | IYPTNGYT | SRWGGDGFYAMDY |
| | | (SEQ ID | (SEQ ID NO: | (SEQ ID NO: |
| | | NO: 431) | 432) | 433) |
| | AbM | GFNIKDTY | RIYPTNGYTR | WGGDGFYAMDY |
| | | IH (SEQ | (SEQ ID NO: | (SEQ ID NO: |
| | | ID NO: | 435) | 428) |
| | | 434) | | |
| Light | Kabat | RASQDVNT | SASFLYS | QQHYTTPPT |
| chain | | AVA (SEQ | (SEQ ID NO: | (SEQ ID NO: |
| | | ID NO: | 437) | 438) |
| | | 436) | | |
| | Chothia | RASQDVNT | SASFLYS | QQHYTTPPT |
| | | AVA (SEQ | (SEQ ID NO: | (SEQ ID NO: |
| | | ID NO: | 437) | 438) |
| | | 436) | | |
| | IMGT | QDVNTA | QQHYTTPPT | |
| | | (SEQ ID | SAS | (SEQ ID NO: |
| | | NO: 439) | | 438) |
| | AbM | RASQDVNT | SASFLYS | QQHYTTPPT |
| | | AVA (SEQ | (SEQ ID NO: | (SEQ ID NO: |
| | | ID NO: | 437) | 438) |
| | | 436) | | |

pertuzumab

| | | | | |
|---|---|---|---|---|
| Heavy | Kabat | DYTMD | DVNPNSGGSI | NIGPSFYFDY |
| chain | | (SEQ ID | YNQRFKG (SEQ | (SEQ ID NO: |
| | | NO: 440) | ID NO: 441) | 442) |
| | Chothia | GFTFTDY | NPNSGG | NLGPSFYFDY |
| | | (SEQ ID | (SEQ ID NO: | (SEQ ID NO: |
| | | NO: 443) | 444) | 442) |
| | IMGT | GFTFTDYT | VNPNSGGS | ARNLGPSFYFDY |
| | | (SEQ ID | (SEQ ID NO: | (SEQ ID NO: |
| | | NO: 445) | 446) | 447) |
| | AbM | GFTFTDYT | DVNPNSGGSI | NLGPSFYEDY |
| | | MD (SEQ | (SEQ ID NO: | (SEQ ID NO: |
| | | ID NO: | 449) | 442) |
| | | 448) | | |
| Light | Kabat | KASQDVSI | SASYRYT | QQYYIYPYT |
| chain | | GVA (SEQ | (SEQ ID NO: | (SEQ ID NO: |
| | | ID NO: | 451) | 452) |
| | | 450) | | |
| | Chothia | KASQDVSI | SASYRYT | QQYYIYPYT |
| | | GVA (SEQ | (SEQ ID NO: | (SEQ ID NO: |
| | | ID NO: | 451) | 452) |
| | | 450) | | |
| | IMGT | QDVSIG | SAS | QQYYIYPYT |
| | | (SEQ ID | | (SEQ ID NO: |
| | | NO: 453) | | 452) |
| | AbM | KASQDVSI | SASYRYT | QQYYIYPYT |
| | | GVA (SEQ | (SEQ ID NO: | (SEQ ID NO: |
| | | ID NO: | 451) | 452) |
| | | 450) | | |

Alternatively, HER2 antigen-binding domains may be derived from novel antibodies that are generated according to methods known in the art.

### SLC39A6/LIV-1 Antigen-Binding Domains

In one embodiment, the 4-1BB x TAA antibody construct is a 4-1BB×LIV-1 antibody construct comprising a 4-1BB antigen-binding domain and a LIV-1 antigen-binding domain wherein the 4-1BB binding domain and the LIV-1 antigen-binding domain are linked directly or indirectly to a scaffold.

SLC39A6, also known as LIV-1 or ZIP6, belongs to a family of proteins that function as zinc transporters. It is expressed at low levels on normal cells throughout the body but is expressed at high levels on some tumor cells, particularly breast cancers (Takatani-Nakase et al., (2016) Biomed Res Clin Prac 1:71-75). The polypeptide sequence of LI V-1 is described in UniProt Accession Number Q13433 and included herein as SEQ ID NO:83.

LIV-1 antigen-binding domains may be derived from antibodies known in the art, including but not limited to those described in WO 2012/078688 (Seattle Genetics), WO 2004/067564 (Abbvie), and WO 2001/055178 (Genentech). Other antibodies that bind to LIV-1 are described in US2008/0175839.

In one embodiment, the 4-1BB×TAA antibody construct comprises a LIV-1 antigen-binding domain that can compete for binding to an epitope of LIV-1 with any one of the antibodies described in WO 2012/078688, WO 2004/067564, WO 2001/055178, or US2008/0175839. In another embodiment, the 4-1BB×TAA antibody construct comprises a LIV-1 that can compete for binding to an epitope of LIV-1 with any one of the anti-LIV-1 antibodies described in WO 2012/078688, WO 2004/067564, WO 2001/055178, or US2008/0175839.

In other embodiments, the 4-1BB×TAA antibody construct comprises a LIV-1 antigen-binding domain that binds to the same epitope of LIV-1. as any one of the anti-LIV-1 antibodies described in WO 2012/078688, WO 2004/067564, WO 2001/055178, or US2008/0175839.

In one embodiment, the 4-1BB×TAA antibody construct comprises an LIV-1 antigen-binding domain that comprises at least one, two, or all three heavy chain CDRs and/or at least one, two, or all three light chain CDRs of any one of the anti-LIV-1 antibodies described in WO 2012/078688, WO 2004/067564, WO 2001/055178, or US2008/0175839. In an alternate embodiment, the 4-11BB×TAA antibody construct comprises an LIV-1 antigen-binding domain that comprises at least one, two, or all three heavy chain CDRs and at least one, two, or all three light chain CDRs of any one of the anti-LIV-1 antibodies described in WO 2012/078688, WO 2004/067564, WO 2001/055178, or US2008/0175839. In other embodiments, the 4-1BB×TAA construct comprises a LIV-1 antigen-binding domain comprising a VH sequence that is at least about 80%, 85%, 90%, 95%, 96%, 97%, 98%, 99%, or 100% identical to the VH sequence of any one of the anti-LIV-1 antibodies described in WO 2012/078688, WO 2004/067564, WO 2001/055178, or US2008/0175839. The specific sequences of the CDRs, VHs, and VLs for exemplary anti-LIV-1 antibodies are described in the disclosures of WO2011/066503, WO2009/097128, or US2017/0266311.

Alternatively, LIV-1 antigen-binding domains may be derived from novel antibodies that are generated according to methods known in the art and described elsewhere herein.

### Mesothelin (MSLN) Antigen-Binding Domains

In one embodiment, the 4-1BB×TAA antibody construct is a 4-1BB×MSLN antibody construct comprising a 4-1BB antigen-binding domain and a MSLN antigen-binding domain wherein the 4-1BB binding domain and the MSLN antigen-binding domain are linked directly or indirectly to a scaffold.

Mesothelin (MSLN), also known as CAK antigen or Pre-pro-megakaryocyte-potentiating factor, is expressed in normal lung mesothelial cells and at low levels in other normal organs. Mesothelin is expressed at high levels in ovarian and lung cancers. The polypeptide sequence of mesothelin is described in UniProt Accession Number Q13421 and included herein as SEQ ID NO:84.

MSLN antigen-binding domains may be derived from anti-MSLN antibodies known in the art, including but not limited to: anetumab (Bayer, described in WO2009/068204), 6A4/BMS-986148 (BMS, described in WO2009/045957), or the Mab Designs anti-MSLN antibody described in WO2018/060480.

In one embodiment, the 4-1BB×TAA antibody construct comprises a MSLN antigen-binding domain that competes for binding with anetumab for binding to MSLN. In another embodiment, the 4-1BB×TAA antibody construct comprises a MSLN antigen-binding domain that competes for binding with 6A4/BMS-986148 for binding to MSLN. In yet another embodiment, the 4-1BB×TAA antibody construct comprises a MSLN antigen-binding domain that competes for binding with the Mab Designs anti-MSLN antibody for binding to MSLN.

In some embodiments, the 4-1BB×TAA antibody construct comprises a MSLN antigen-binding domain that binds to the same epitope as anetumab for binding to MSLN. In another embodiment, the 4-1BB×TAA antibody construct comprises a MSLN antigen-binding domain that binds to the same epitope as 6A4/BMS-986148 for binding to MSLN. In yet another embodiment, the 4-1BB×TAA antibody construct comprises a MSLN antigen-binding domain that binds to the same epitope as the Mab Designs anti-MSLN antibody for binding to MSLN.

In one embodiment, the 4-1BB×TAA antibody construct comprises a MSLN antigen-binding domain that comprises at least one, two, or all three heavy chain CDRs and/or at least one, two, or all three light chain CDRs of any one of the anti-MSLN antibodies described in WO2009/068204, WO2009/045957, or WO2018/060480. In an alternate embodiment, the 4-1BB×TAA antibody construct comprises a MSLN antigen-binding, domain that comprises at least one, two, or all three heavy chain CDRs and at least one, two, or all three light chain CDRs of any one of the anti-MSLN antibodies described in WO2009/068204, WO2009/045957, or WO2018/060480. In other embodiments, the 4-1BB×TAA construct comprises a MSLN antigen-binding domain comprising a VH sequence that is at least about 80%, 85%, 90%, 95%, 96%, 97%, 98%, 99%, or 100% identical to the VH sequence of any one of the anti-MSLN antibodies described in WO2009/068204, WO2009/045957, or WO2018/060480. The specific sequences of the CDRs of exemplary anti-MSLN antibodies are described in Table the VH and VL sequences of these antibodies are found in Table 17. Other anti-MSLN antibody sequences can readily be determined by one of skill in the art with reference to the disclosures of WO2009/068204, WO2009/045957, or WO2018/060480.

TABLE F
CDR sequences of an exemplary anti-MSLN antibody
RG7787

| | Defi- | | | |
|---|---|---|---|---|
| | nition | CDR1 | CDR2 | CDR3 |
| Heavy | Kabat | GYTMN | LITPYNGASS | GGYDGRGFDY |
| Chain | | (SEQ ID | NYQKFRG | (SEQ ID NO: |
| | | NO: 454) | (SEQ ID NO: | 456) |
| | | | 455) | |
| | Chothia | GYSFTGY | TPYNGA | GGYDGRGFDY |
| | | (SEQ ID | (SEQ ID NO: | (SEQ ID NO: |
| | | NO: 457) | 458) | 456) |
| | IMGT | GYSFTGYT | ITPYNGAS | ARGGYDGRGFDY |
| | | (SEQ ID | (SEQ ID NO: | (SEQ ID NO: |
| | | NO: 459) | 460) | 461) |
| | AbM | GYSFTGYTMN | LITPYNGASS | GGYDGRGFDY |
| | | (SEQ ID | (SEQ ID NO: | (SEQ ID NO: |
| | | NO: 462) | 463) | 456) |
| Light | Kabat | SASSSVSYMH | DTSKLAS | QQWSKHPLT |
| Chain | | (SEQ ID | (SEQ ID NO: | (SEQ ID NO: |
| | | NO: 464) | 465) | 466) |
| | Chothia | SASSSVSYMH | DTSKLAS | QQWSKHPLT |
| | | (SEQ ID | (SEQ ID NO: | (SEQ ID NO: |
| | | NO: 464) | 465) | 466) |
| | IMGT | SSVSY | DTS | QQWSKHPLT |
| | | (SEQ ID | (SEQ ID NO: | |
| | | NO: 467) | 466) | |
| | AbM | SASSSVSYMH | DTSKLAS | QQWSKHPLT |
| | | (SEQ ID | (SEQ ID NO: | (SEQ ID NO: |
| | | NO: 464) | 465) | 466) |

### Scaffolds

As described herein, the 4-1BB×TAA antibody construct comprises a 4-1BB binding domain that binds to a 4-1BB ECD and a TAA antigen-binding domain, wherein the first 4-MB binding domain and the TAA antigen-binding domain are linked directly or indirectly to a scaffold. Direct linkage of the 4-1BB-binding domain and the TAA antigen-binding domain results when each of these domains is directly linked to the scaffold, without linkers. Thus, in one embodiment, the 4-1BB-binding domain is linked to the scaffold without a linker and the TAA antigen-binding domain is also linked to the scaffold without a linker. Methods of accomplishing direct linkage are known in the art and include recombinant DNA methodology and/or chemical conjugation, for example.

Indirect linkage can be achieved by using a linker to link one or both of the 4-1BB-binding domain and the TAA antigen-binding domain to the scaffold. Accordingly, in one embodiment, the 4-1BB-binding domain is linked to the scaffold with a linker and the TAA antigen-binding domain is also linked to the scaffold with a linker. In other embodiments, one of the 4-1BB-binding domain and the TAA antigen-binding domain is linked to the scaffold with a linker and the other is directly linked to the scaffold without a linker. In still other embodiments, the 4-1BB-binding domain is linked to the scaffold with a linker, and the TAA antigen-binding domain is linked to the 4-1BB-binding domain with a linker. In the latter embodiment, the TAA antigen-binding domain is considered to be indirectly linked to the scaffold. In an alternate embodiment, the TAA antigen-binding domain is linked to the scaffold with a linker, and the 4-1BB-binding domain is linked to the TAA antigen-binding domain with a linker. In the latter embodiment, the 4-1BB-binding domain is considered to be indirectly linked to the scaffold.

### Linkers and Linker Polypeptides

As indicated above, in some embodiments, indirect linkage of the 4-1BB-binding domains and the TAA antigen-binding domain to the scaffold is accomplished by the use of linkers. The linker may be a linker peptide, a linker polypeptide, or a non-polypeptide linker. In some embodiments, the antibody constructs described herein include a 4-1BB-binding domain and a TAA antigen-binding domain that are each operatively linked to a linker polypeptide wherein the linker polypeptides are capable of forming a complex or interface with each other. In some embodiments, the linker polypeptides are capable of forming a covalent linkage with each other. The spatial conformation of the constructs with the linker polypeptides is similar to the relative spatial conformation of the paratopes of a F(ab′)2 fragment generated by papain digestion, albeit in the context of an antibody construct with two antigen-binding domains.

In one embodiment, the linker polypeptides are selected from IgG1, IgG2, IgG3, or IgG4 hinge regions.

In some embodiments, the linker polypeptides are selected such that they maintain the relative spatial conformation of the paratopes of a F(ab′) fragment and are capable of forming a covalent bond equivalent to the disulphide bond in the core hinge of IgG. Suitable linker polypeptides include IgG hinge regions such as, for example those from IgG1IgG2, or IgG4. Modified versions of these exemplary linkers can also be used. For example, modifications to improve the stability of the IgG4 hinge are known in the art (see for example, Labrijn et al. (2009) Nature Biotechnology 27, 767-771),

A number of suitable scaffolds are known in the art, including peptides, polypeptides, polymers, nanoparticles or other chemical entities. In one embodiment, the scaffold is an Fc construct, A number of scaffolds based on alternate protein or molecular domains are known in the art and can be used to form selective pairs of two different target-binding polypeptides. Examples of such alternate domains include the cohesin-dockerin scaffolds described International Patent Publication No. WO2008/097817, and the split albumin scaffolds described in WO 2012/116453 and WO 2014/012082. A further example is the leucine zipper domains such as Fos and Jun that selectively pair together [S A Kostelny et al. J Immunol 1992 148:1547-53; Bernd J. Wranik, et al. J. Biol. Chem. 2012 287: 43331-43339]. Alternately, other selectively pairing molecular pairs such as the barnase barstar pair [Deyev, et al. (2003). Nat Biotechnol 21, 1486-1492], or split fluorescent protein pairs [WO 2011135040] can also be employed.

In other embodiments, the linker polypeptides are operatively linked to scaffolds other than an Fc, A number of scaffolds based on alternate protein or molecular domains are known in the art and can be used to form selective pairs of two different target-binding polypeptides. Examples of such alternate domains are the split albumin scaffolds described in WO 2012/116453 and WO 2014/012082. A further example is the leucine zipper domains such as Fos and Jun that selectively pair together [S A Kostelny, M S Cole, and J Y Tso. Formation of a bispecific antibody by the use of leucine zippers. J Immunol 1992 148:1547-53; Bernd. J. Wranik, Erin L. Christensen, Gabriele Schaefer, Janet K. Jackman, Andrew C. Wendel, and Eaton. LUZ-Y, a Novel Platform for the Mammalian Cell Production of Full-length IgG-bispecific Antibodies J. Biol. Chem. 2012 287: 43331-43339], Alternately, other selectively pairing molecular pairs such as the barnase barstar pair [Deyev, S. M., Waibel, R., Lebedenko, E. N., Schubiger, A. P., and Plïckthunk. (2003). Design of multivalent complexes using the barnase*barstar module, Nat Biotechnol 21, 1486-1492], DNA strand pairs [Zahida N. Chaudri, Michael Bartlet-Jones, George Panayotou, Thomas Klonisch, Ivan M. Roitt, Torben Lund, Peter J. Delves, Dual specificity antibodies using a double-stranded oligonucleotide bridge, FES Letters, Volume 450, Issues 1-2, 30 Apr. 1999, Pages 23-26], split fluorescent protein pairs [Ulrich Brinkmann, Alexander Haas. Fluorescent antibody fusion protein, its production and use, WO 2011135040 A1] can also be employed.

In embodiments where the scaffold is a peptide or polypeptide, the 4-1BB-binding domain and/or the TAA antigen-binding domain of the antibody construct may be linked directly or indirectly to the scaffold by genetic fusion. In other embodiments, where the scaffold is a polymer or nanoparticle, the 4-1BB-binding domain and/or the TAA antigen-binding domain of the antibody construct may be linked to the scaffold by chemical conjugation.

In one embodiment, the antibody construct described herein comprises a 4-1BB-binding domain, and a tumor-associated antigen (TAA)-antigen binding domain, wherein the first 4-1BB-binding domain and the TAA antigen binding domain are linked directly or indirectly to an Fc construct.

The term “Fc” or “Fc construct” as used herein refers to a C-terminal region of an immunoglobulin heavy chain that contains at least a portion of the constant region (also referred to as an “Fc domain” or “Fc region”), including the CH3 domain. The term includes native sequence Fc regions and variant Fc regions, Unless otherwise specified herein, numbering of amino acid residues in the Fc region or constant region is according to the EU numbering system, also called the EU index, as described in Edelman, G. M. of al., Proc. Natl. Acad. USA, 63, 78-85 (1969).

A “dimeric Fc construct” comprises two Fc polypeptides. An “Fc polypeptide” of a dimeric Fc construct refers to one of the two polypeptides forming the construct, i.e. a polypeptide comprising C-terminal constant regions of an immunoglobulin heavy chain that is capable of stable self-association. The Fc polypeptides are derived from heavy chain isotypes including IgG, IgA, IgM, 1gD, and IgE. The Fc polypeptides may also he derived from the heavy chain subtypes IgG1, IgG2, IgG3, IgG4, IgA 1, or IgA2. In some embodiments, the Fc construct is a human Fc construct. In some embodiments, the Fc construct is a human IgG Fc construct. In other embodiments, the Fc construct is a human IgG1 Fc construct.

Each Fc polypeptide comprises a CH3 sequence and may optionally comprise a CH2 sequence. In some embodiments, each Fc polypeptide comprises a CH3 sequences having one or more amino acid modifications. In some embodiments, each Fc polypeptide comprises a CH2 sequence comprises one or more amino acid modifications. In some embodiments, an Fc construct is composed of a single polypeptide, for example where the Fc polypeptides are linked by a linker. In other embodiments, the Fc construct is a heterodimeric Fc construct, wherein the Fc polypeptides that make up the Fc construct have different CH3 or CH2 sequences.

### CH3 Sequence Modifications

In certain embodiments, the scaffold is a heterodimeric Fc construct comprising CH3 sequence modifications that promote the formation of a heterodimeric Fc construct compared to a homodimeric Fc, as described in International Patent Application No. PCT/CA2011/001238 or International Patent Application No. PCT/CA2012/050780, the entire disclosure of each of which is hereby incorporated by reference in its entirety for all purposes.

Table G provides the amino acid sequence of the human IgG1 Fc sequence, corresponding to amino acids 231 to 447 of the full-length human IgG1 heavy chain. The CH3 sequence comprises amino acid 341-447 of the full-length human IgG1 heavy chain.

Typically, an Fc includes two contiguous heavy chain sequences or Fc polypeptide sequences (A and B) that are capable of dimerizing. In some embodiments, one or both sequences of these sequences may include one or more mutations or modifications at the following locations: L351, F405, Y407, T366, K392, T394, T350, S400, and/or N390, using EU numbering. In some embodiments, an Fc may include a mutant sequence as shown in Table G. In some embodiments, an Fc may include the mutations of Variant 1 A-B. In some embodiments, an Fc may include the mutations of Variant 2 A-B. In some embodiments, an Fc may include the mutations of Variant 3 A-B. In some embodiments, an Fc may include the mutations of Variant 4 A-B. In some embodiments, an Fc may include the mutations of Variant 5 A-B.

TABLE G
IgG1 Fc sequences

| Human IgG1 Fc | APELLGGPSVFLFPPKPKDTLMISRTPEVTCVVVDVS | |
|---|---|---|
| sequence 231- | HEDPEVKFNWYVDGVEVHNAKTKPREEQYNSTYRVVS | |
| 447 (EU- | VLTVLHQDWLNGKEYKCKVSNKALPAPIEKTISKAKG | |
| numbering) | QPREPQVYTLPPSRDELTKNQVSLTCLVKGFYPSDIA | |
| | VEWESNGQPENNVKTTPPVLDSDGSFFLYSKLTVDKS | |
| | RWQQGNVFSCSVMHEALHNHYTQKSLSLSPGK | |
| | (SEQ ID NO: 85) | |
| Variant IgG1 | | |
| Fc sequence | | |
| (231-447) | Chain | Mutations |
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

Additional methods for modifying the Fc polypeptides of the Fc construct to promote heterodimeric Fc formation are known in the art and include, for example, those described in International Patent Publication No. WO 96/027011 (knobs into holes), in Gunasekaran et al. (Gunasekaran K. et al. (2010) J Biol Chem. 285, 19637-46, electrostatic design to achieve selective heterodimerization), in Davis et al. (Davis, J H et al, (2010) Prot Eng Des Sel :23(4): 195-202, strand exchange engineered domain (SEED) technology), and in Labrijn et al [Efficient generation of stable bispecific IgG1 by controlled Fab-arm exchange. Labrijn A F, Meesters J I, de Goeij B E, van den Bremer Neijssen J, van Kampen M D, Strumane K, Verploegen S, Kundu A, Gramer M J, van Berkel P H, van de Winkel J G, Schuurman J, Parren P W. Proc Natl Acad Sci U S A. 2013 Mar. 26; 1.10(13):5145-50.

### CH2 Sequence Modifications

In some embodiments, the scaffold is an construct wherein each Fc polypeptide of the Fc construct comprises a CH2 sequence and a CH3 sequence. One example of a CH2 sequence of an Fc is amino acids 231-340 of the sequence shown in Table B. Several effector functions are mediated by Fc receptors (FcRs), which bind to the Fc of an antibody.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor” and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab, Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 sequence can affect the binding of FcRs to the Fc construct. A number of amino acid modifications in the Fc region are known in the art for selectively altering the affinity of the Fc for different Fcgamma receptors. In some aspects, the Fc comprises one or more modifications to promote selective binding of Fc-gamma receptors.

Exemplary mutations that alter the binding of FcRs to the Fc are listed below:
S298A/E333A/K334A, S298A/E333A/K334A/K326A (Lu. Y, Venues J M, Chiang N, et al. J Immunol Methods. 2011 Feb. 28; 365(1-2):132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13(6):R123);
F243L (Stewart R, Thom G, Levens M, et al. Protein Eng Des Sel. 2011 September; 24(9):671-8.)
S298A/E333A/K334A (Shields R L, Namenuk A K, Hong K, et al. J Biol Chem. 2001 Mar. 2; 276(9):6591-604);
S239D/I1332E/A330L, S239D/I332E (Lazar G A, Dang W, Karki S, et al. Proc Natl Acad Sci USA. 2006 Mar. 14; 103(11):4005-10);
S239D/S267E, S267E/L328F (Chu S Y, Vostiar I, Karki S. et al. Mol Immunol. 2008 September; 45(15):3926-33);
S239D/D265S/S298A/I332E, S239E/S298A/K326A/A327H, G237F/S298A1A330L/I 332, S239D/I332E/S298A, S239D/K326E/A330L/I332E/S298A, G236A/S239D/D27 0LI332E, S239E/S267E/H268D, L234F/S267E/N325L, G237F/V266L/S267D and other mutations listed in WO2011/120134 and WO2011/120135, herein incorporated by reference.

Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, the heterodimeric Fc comprises Fc polypeptides having CH2 sequences comprising one or more asymmetric amino acid modifications. Exemplary asymmetric amino acid modifications are described in international Patent Application No. PCT/CA2014/050507. In one embodiment the heterodimeric Fc comprises Fc polypeptides having the amino acid substitutions L234A, L235A, and D265S which reduce FcγR binding.

### Additional Modifications to Improve Effector Function

In some embodiments, the Fc construct includes amino acid modifications that improve its ability to mediate effector function. Such modifications are known in the art and include afucosylation, or engineering of the affinity of the towards an activating receptor, mainly FCγRIIIa for ADCC, and towards Clq for CDC.

Methods of producing antibody Fc regions with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody Fc region production. This prevents the addition of the sugar “fucose” to the N-linked antibody carbohydrate part by cells. (von Horsten et al. (2.010) Glycobiology. 20 (12):1607-18). Another approach to obtaining antibody constructs having Fc constructs with lowered levels of fucosylation can be found in U.S. Pat. No. 8,409,572, which teaches selecting cell lines for antibody production based on their ability to yield lower levels of fucosylation on antibodies. In some embodiments, the Fc of the antibody constructs or antibody constructs can be fully afucosylated (meaning they contain no detectable fucose) or they can be partially afucosylated, meaning that the TAA presentation inducer in bispecific antibody format contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

Thus, in some embodiments, the antibody constructs described herein can include a dimeric Fc that comprises one or more amino acid modifications as noted in Table H that confer improved effector function. In some embodiments, the construct can be afucosylated to improve effector function.

TABLE H
CH2 domains and effector function engineering

| Reference | Mutations | Effect |
|---|---|---|
| Lu, 2011, | Afucosylated | Increased ADCC |
| Ferrara 2011, | | |
| Mizushima 2011 | | |
| Lu, 2011 | S298A/E333A/K334A | Increased ADCC |
| Lu, 2011 | S298A/E333A/K334A/K326A | Increased ADCC |
| Stavenhagen, 2007 | F243L/R292P/Y300L/V305I/ | Increased ADCC |
| | P396L | |
| Nordstrom, 2011 | F243L/R292P/Y300L/L235V/ | Increased ADCC |
| | P396L | |
| Stewart, 2011 | F243L | Increased ADCC |
| Shields, 2001 | S298A/E333A/K334A | Increased ADCC |
| Lazar, 2006 | S239D/I332E/A330L | Increased ADCC |
| Lazar, 2006 | S239D/I332E | Increased ADCC |
| Bowles, 2006 | AME-D, not specified mutations | Increased ADCC |
| Heider, 2011 | 37.1, mutations not disclosed | Increased ADCC |
| Moore, 2010 | S267E/H268F/S324T | Increased CDC |

Fc modifications reducing FcγR and/or complement binding and/or effector function are known in the art. Various publications describe strategies that have been used to engineer antibodies with reduced or silenced effector activity (see Strobl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al ((2012) J. Mol. Biol. 420: 204-219) describe specific modifications to reduce FcγR or complement binding to the Fc.

Specific, non-limiting examples of known amino acid modifications to reduce FcγR or complement binding to the Fc include those identified in Table I.

TABLE I
Modifications to reduce FcγR or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glyco |
| Medimune | L234F/L235E/P331S |
| Trubion | Hinge mutant, possibly C226S/P230S |

In some embodiments, the Fc comprises at least one amino acid modification identified in Table I. In some embodiments, the Fc comprises amino acid modification of at least one of L234, L235, or D265. In some embodiments, the Fc comprises amino acid modification at L234, L235 and D265. in some embodiments, the Fc comprises the amino acid modification L234A, L235A and D265S.

In embodiments where the scaffold is an Fc, the 4-1BB-binding domain may be linked to the N-terminus of one of the Fc polypeptides. In other embodiments, the 4-1BB-binding domain may be linked to the C-terminus of one of the Fc polypeptides. In certain embodiments, the 4-1BB×TAA antibody construct can comprise a 4-1BB-binding domain linked to the N-terminus of one of the Fc polypeptides and another 4-1BB-binding domain linked to the N-terminus of the other Fc polypeptide. In yet other embodiments, the 4-1BB×TAA antibody construct can comprise a 4-MB-binding domain that is linked to the C-terminus of one of the Fc polypeptides. In certain embodiments, the 4-1BB×TAA antibody construct can comprise a 4-1BB-binding domain linked to the C-terminus of one of the Fc polypeptides and another 4-1RB-binding domain linked to the C-terminus of the other Fc polypeptide.

In additional embodiments where the scaffold is an Fc, the TAA antigen-binding domain may be linked to the N-terminus of one of the Fc polypeptides. In other embodiments, the TAA antigen-binding domain may be linked to the C-terminus of one of the Fc polypeptides. In certain embodiments, the 4-1BB×TAA antibody construct can comprise a TAA antigen-binding domain linked to the N-terminus of one of the Fc polypeptides and another TAA antigen-binding domain linked to the N-terminus of the other Fc polypeptide. In yet other embodiments, the 4-1BB×TAA can comprise a TAA antigen-binding domain that is linked to the C-terminus of one of the Fc polypeptides. In certain embodiments, the 4-1BB×TAA antibody construct can comprise a TAA antigen-binding domain linked to the C-terminus of one of the Fc polypeptides and another TAA antigen-binding domain linked to the C-terminus of the other Fc polypeptide.

As would be understood by one of skill in the art, in some embodiments combinations of the above linkages are also possible. Specific exemplary combinations are described as follows.

### Formats of Antibody Constructs

### 4-1.BB×TAA Antibody Constructs

The 4-1BB×TAA antibody constructs described herein comprise a 4-1BB-binding domain and a TAA antigen-binding domain wherein the 4-1BB-binding domain and the TAA antigen-binding domain are linked directly or indirectly to a scaffold. As is known in the art, these 4-1BB×TAA antibody constructs may be constructed in many formats; exemplary, non-limiting formats are described below.

In embodiments where the 4-1BB-binding domain of the 4-1BB×TAA antibody construct is a 4-1BB antigen-binding domain, the 4-1BB antigen-binding domain may be in Fab format, scFv format, or sdAb format. In one embodiment, the antibody construct comprises a 4-1BB antigen-binding domain that is in the Fab format, and a TAA antigen-binding domain, wherein the 4-1BB antigen-binding domain and the TAA antigen-binding domain are linked directly or indirectly to a scaffold. In another embodiment, the antibody construct comprises a 4-1BB antigen-binding domain that is in the scFv format, and a TAA antigen-binding domain, wherein the 4-1BB antigen-binding domain and the TAA antigen-binding domain are linked directly or indirectly to a scaffold. In one embodiment, the antibody construct comprises a 4-1BB antigen-binding domain that is in the sdAb format, and a TAA antigen-binding domain, wherein the 4-1BB antigen-binding domain and the TAA antigen-binding domain are linked directly or indirectly to a scaffold. In some of these embodiments, the 4-1BB antigen-binding domain is linked to the N-terminus of the scaffold and the TAA antigen-binding domain is linked to the C-terminus of the scaffold. In other embodiments, both the 4-1BB antigen-binding domain and the TAA antigen-binding domain are linked to the N-terminus of the scaffold.

In some embodiments, the scaffold is an Fc construct. In one such embodiment, the 4-1BB×TAA antibody construct comprises a first 4-1BB antigen-binding domain linked to the N-terminus of a first Fc polypeptide, a second 4-1BB antigen-binding domain linked to the N-terminus of a second Fc polypeptide, and a TAA antigen-binding domain linked to the C-terminus of the first Fc polypeptide. In some embodiments, the first and second 4-1BB antigen-binding domains are both in the Fab format and the TAA antigen-binding domain is in the scFv format. FIG. 2B provides a representation of an exemplary construct related to these embodiments.

In other embodiments where the scaffold is an Fc construct, the 4-1BB×TAA antibody construct comprises a first 4-1BB antigen-binding domain linked to the N-terminus of a first Fc polypeptide, a second 4-i BB antigen-binding domain linked to the N-terminus of a second Fc polypeptide, a first TAA antigen-binding domain linked to the C-terminus of the first Fc polypeptide, and a second TAA linked to the C-terminus of the second Fc polypeptide. In some embodiments, the first and second 4-1BB antigen-binding domains are both in the Fab format and the first and second TAA antigen-binding domain are both in the scFv format. FIG. 2C provides a representation of an exemplary construct related to these embodiments.

In other embodiments where the scaffold is an Fc construct, the 4-1BB×TAA antibody construct comprises a 4-1BB antigen-binding domain linked to the N-terminus of one of the Fc polypeptides of the Fc construct, and a first TAA antigen-binding domain linked to the C-terminus of the same Fc polypeptide. In some embodiments, the 4-1BB antigen-binding domain is in the Fab format and the TAA antigen-binding domain is in the scFv format. FIG. 2D provides a representation of an exemplary construct related to these embodiments.

In still other embodiments where the scaffold is an Fc construct, the 4-1BB×TAA antibody construct comprises a first 4-1BB antigen-binding domain linked to the N-terminus of a first Fc polypeptide, a second 4-1BB antigen-binding domain linked to the N-terminus of a second Fc polypeptide, and a TAA antigen-binding domain linked to the N-terminus of the VH domain of the first 4-1BB antigen-binding domain. In some embodiments, the first and second 4-1BB antigen-binding domains are both in the Fab format and the TAA antigen-binding domain is in the scFv format. FIG. 2E provides a representation of an exemplary construct related to these embodiments.

In other embodiments where the scaffold is an Fc construct, the 4-1BB×TAA antibody construct comprises a 4-1BB antigen-binding domain linked to the N-terminus of a first Fc polypeptide, and a TAA antigen-binding domain linked to the N-terminus of the second Fc polypeptide. In some embodiments, the 4-1BB antigen-binding domains are both in the Fab format and the TAA antigen-binding domain is in the scFv format. FIG. 2F provides a representation of an exemplary construct related to these embodiments.

In other embodiments where the scaffold is an Fc construct, the 4-1BB×TAA antibody construct comprises a 4-1BB antigen-binding domain linked to the N-terminus of one of the Fc polypeptides of the Fc construct and a TAA antigen-binding domain linked to the N-terminus of the VH region of the 4-1BB antigen-binding domain. In some embodiments, the 4-1BB antigen-binding domain is in the Fab format and the TAA antigen-binding domain is in the say format. FIG. 2G provides a representation of an exemplary construct related to these embodiments.

In further embodiments where the scaffold is an Fc construct, the 4-1BB×TAA antibody construct comprises a 4-1BB antigen-binding domain linked to the N-terminus of one of the Fc polypeptides of the Fc construct, a TAA antigen-binding domain linked to the C-terminus of the same Fc polypeptide, and a TAA antigen-binding domain linked to the C-terminus of the second Fc polypeptide. In some embodiments, the 4-1BB antigen-binding domain is in the Fab format and the TAA antigen-binding domains are in the scFv format. FIG. 2G provides a representation of an exemplary construct related to these embodiments.

In yet other embodiments where the scaffold is an Fc construct, the 4-1BB×TAA antibody construct comprises a 4-1BB antigen-binding domain linked to the N-terminus of one of the Fc polypeptides of the Fc construct, and a TAA antigen-binding domain linked to the C-terminus of the second Fc polypeptide. In some embodiments, the 4-1BB antigen-binding domain is in the Fab format and the TAA antigen-binding domain is in the scFv format.

In one embodiment, the 4-1BB×TAA antibody construct comprises a 4-1BB-binding domain that is a 4-1BB ligand, the 4-1BB ligand linked to the C-terminus of one of the Fc polypeptides of an Fc construct, and a TAA antigen-binding domain that is in a Fab format, linked to the N-terminus of the other Fc polypeptide of the Fc construct.

### Functional Activity of the 4-1BB×TAA Antibody Constructs

The 4-1BB×TAA antibody constructs provided herein can bind 4-1BB and a TAA with a range of affinities. The affinity or avidity of an antibody for an antigen can be determined experimentally using methods known in the art (see, for example, Berzofsky, et al., “Antibody-Antigen Interactions,” In Fundamental Immunology, Paul. W. Ed., Raven Press: New York, N.Y. (1984); Kuby, Janis Immunology, W.H. Freeman and Company: New York, N.Y. (1992); and methods described herein).

The measured affinity of a particular antibody-antigen interaction can vary if measured under different conditions (e.g., salt concentration, pH). Thus, measurements of affinity and other antigen-binding parameters are preferably made with standardized solutions of antibody and antigen, and a standardized buffer, such as the buffer described herein. The affinity, K_{D} is a ratio of kₒₙ/k_{off}. Generally, a K_{D} in the micromolar range is considered low affinity for monospecific bivalent antibodies. Generally, a K_{D} in the picomolar range is considered high affinity for monospecific bivalent antibodies. As is known in the art, the affinity of an antibody measured as a monovalent binder, is generally lower than that measured as a bivalent binder.

In some embodiments, the 4-11.13B x TAA antibody construct comprises a 4-1BB antigen-binding domain that has an K_{D} for human 4-1BB between about 15 nM and 100 nM, about 15 nM and 200 nM, or about 15 nM and 500 nM, between about 100 pM and 1 μM, measured as a monovalent binder. In additional embodiments, the 4-1BB×TAA antibody construct comprises a 4-1BB antigen-binding domain that has an KD for human 4-1BB between about 1 nM and about 1000 nM, or between about 10 nM to about 500nM, or between about 20 nM and about 400 nM measured as a monovalent binder. In other embodiments, the 4-1BB×TAA antibody construct comprises a TAA antigen-binding domain having a K_{D} for the TAA of between about 0.1 nM to about 50 nM, or about 1 nM to about 20 nM, or about 1 nM to about 10 nM. The KD may be measured by a number of known methods, for example SPR, as described elsewhere herein. As used in this section, the term “about” means 10% of the value for K_{D} identified in each range.

In some embodiments, the 4-1BB×TAA antibody construct binds to one or more TAA-expressing cell lines as determined by for example, ELBA, BiaCore™, and/or flow cytometry, or as described in the Examples. In certain embodiments, the TAA-expressing cell line is an ovarian adenocarcinoma cell line, such as, for example, IGROV1, SKOV3, or OVCAR3. In certain embodiments, the TAA-expressing cell line is a lung carcinoma cell line. In certain embodiments, the lung carcinoma cell line is a lung squamous cell line such as H226; or a lung adenocarcinoma cell line such as H441, HCC827, H1573, H1975, orE11563; or a lung carcinoma cell line such as H1299, or a lung large cell carcinoma, such as H661. In some embodiments the TAA-expressing cell line is a HER2-expressing cell line such as SKBr3, a FRα-expressing cell line, a LIV-1-expressing cell line, an NaPi2b-expressing cell line, or a mesothelin-expressing cell line.

In some embodiments, the 4-1BB×TAA antibody construct may be able to stimulate 4-1BB activity in T cells as measured by cytokine production, in the presence of TAA expressing cells. In some embodiments, the TAA-expressing cell expresses the TAA on the cell surface at greater than about 500,000 molecules per cell, as measured by quantitative flow cytometry or other quantitative method. In some embodiments, the TAA-expressing cell expresses the TAA on the cell surface at greater than about 100,000 molecules per cell, as measured by quantitative flow cytometry or other quantitative method. In some embodiments, the TAA-expressing cell expresses the TAA on the cell surface at between about 100,000 and 500,000 molecules per cell, as measured by quantitative flow cytometry or other quantitative method. In some embodiments, the TAA-expressing cell expresses the TAA on the cell surface at between about 50,000 and 500,000 molecules per cell, as measured by quantitative flow cytometry or other quantitative method.

In some embodiments, the 4-1BB×TAA antibody construct binds to 4-1BB-expressing cells as determined by the methods described above. In some embodiments, the 4-1BB-expressing cells are primary T, NK or NKT cells, activated primary T, NK or NKT cells, regulatory T cells, or T, NKT or NKT cells extracted from tumours.

In some embodiments, the 4-1BB×TAA antibody constructs described herein may be capable of stimulating 4-1BB signalling in 4-1BB expressing cells. Methods of testing for 4-1BB activity are known in the art. For example, an NF-κB reporter gene assay as described in the examples may be used to assess the ability of the 4-1BB×TAA antibody constructs to promote NF-κB activation and translocation to the nucleus, subsequently driving reporter gene expression. As another example, a primary T cell coculture assay as described in the examples may be employed to assess the ability of the 1-1BB×TAA antibody constructs to stimulate T cell activation by measuring the increase or decrease in the production of a cytokine (such as IL-2, IL-4, IL-5, IL-6, IL-9, IL-10, IL-12, IL-13, IL-15, IL-21, IL-22, IL-35, IFNγ, TNF-α, TGF-β), increase or decrease in the expression of chemokine receptors (CXCR3, CXCRS, CXCR6, CCR1, CCR2, CCR4, CCRS, CCR7, CCR8, CCR9, CCR10), increase or decrease in expression of key transcription factors (Tbet, GATA3, FOXP3, EOMES, TOX), increase or decrease in metabolic activity or proteins regulating metabolic activity, increase or decrease in expression of anti- or pro-apoptotic proteins (Bcl2, Bcl-Xl, Bim, Mcl1), increase or decrease in expression of surface markers (PD1, TIGIT, LAG3, ICOS, CD45RA, CD45RO, CD44, :D69, CD44, KLRG1), increase or decrease in the ability of T cells to kill tumour cells, or the phosphorylation, localization or activity of signaling proteins (Akt/PkB, PI3K, CD3zeta, LAT, SLP76, IκK, NFκB, TRAF family MEK, MEKK, NIK, ERK, p38 MAPK, c-fos, c-jun, ATF, Foxo) or proteins regulating cell cycle (CyclinD3, p27kip1). This could be assessed either at the level of protein, mRNA or chromosomal availability. Activity in the primary T cell assay could also be assessed by examining increase or decrease in total cellular DNA contents (by measuring incorporation of ³H-thymidine, bromodeoxyuridine or analogous trackers) or by increase or decrease in levels of a tracking dye which is able to determine number of divisions in assays where the cells are labelled with the dye (CFDA-SE, Cell Tracker Violet, PKH26). These assays are well known to one of skill in the art, and in many cases, reagents and kits for carrying out these assays are commercially available, such as for example, CellTracker™Violet BMQC Dye (Thermofisher Scientific) or CellTrace™ Violet Cell proliferation Kit, Thermofisher Scientific/Invitrogen™.

### 4-1BB Antibody Constructs

The present disclosure further provides antibody constructs or antigen-binding fragments thereof that specifically bind to 4-1BB ECD (4-1BB antibody constructs). In some embodiments, these 4-1BB antibody constructs comprise VH and VL sequences as set forth in Tables 13 and 14, and the CDR sequences of these VH and VL sequences can be found in Table 18. In certain embodiments, the 4-1BB antibody construct is capable of agonizing 4-1BB activity as described elsewhere herein. In some embodiments the 4-1BB antibody construct binds to any one of CRD1, CRD2, CRD3, or CDR4 of human 4-1BB.

In one embodiment, a 4-1BB antibody construct or antigen-binding fragment thereof, comprises a heavy chain variable sequence comprising three heavy chain CDRs and a light chain variable sequence comprising three light chain CDRs, wherein the heavy chain CDRs and the light chain CDRs are from any one of antibodies IG1, 1B2, 1C3, 1C8, 2A7, 2E8, 2H9, 3D7, 3H1, 3E7, 3G4, 4B11 4E6, 4F9, G10, 5E2, 5G8, or 6B3 and the 4-1BB antibody construct binds to human 4-1BB. In some embodiments, the 4-1BB antibody construct or antigen-binding fragment comprises a heavy chain variable (VH) sequence comprising three CDRs and a light chain variable (VL) sequence comprising three CDRs, wherein the heavy chain CDRs and the light chain CDRs are from any one of antibodies 1G1, 1C3, 1C8, 2E8, 3E7, 4E6, 5G8, or 6B3, and the 4-1BB antibody construct binds to human 4-1BB.

In certain embodiments, the 4-1BB antibody construct comprises VH and VL sequences that are human or humanized. In other embodiments, the 4-1BB antibody construct comprises a VH sequence and a VL sequence having at least 85%, 90%, 95%, 96%, 97%, 98%, or 99% sequence identity to the VH and VL sequences of any one of variants v28726, v28727, v28728, v28730, v28700, v28704, v28705, v28706, v28711, v28712, v28713, v28696, v28697, v28698, v28701, v28702, v28703, v28707, v28683, v28684, v28685, v28686, v28687, v28688, v28689, v28690, v28691, v28692, v28694, or v28695.

The anti-4-1BB CDR, VH and VL sequences may be used to construct various formats of antibody constructs as is known in the art. For example, these sequences may be used to construct Fab fragments or says, which may be linked to a scaffold such as an Fc or other scaffolds as described herein. Exemplary formats for antibody constructs comprising these CDR, VH and VL sequences are depicted in FIG. 1. The antibody constructs may be monovalent, bivalent or multivalent. In some embodiments, the antibody constructs are monospecific. In some embodiments, the antibody constructs are monospecific and in the naturally occurring format (FSA).

The anti-4-1BB VH and VI, sequences as set forth in Tables 13, 14 and the anti-4-1BB CDR sequences found in Table 18 may further be used in the construction of bispecific or multispecific antibodies, such as the 4-1BB×TAA antibody constructs described here, or other antibody constructs comprising at least one antigen-binding domain that binds to the ECD of 4-1BB.

In some embodiments, the 4-1BB antibody constructs in monovalent form bind human 4-1BB with a K_{D} for 4-1BB between about 15 nM and 100 nM, about 15 nM and 200 nM, or about 15 nM and 500 nM, between about 100 pM and 1 μM. In additional embodiments, the 4-1BB antibody construct in monovalent form comprises a 4-1BB antigen-binding domain that has an K_{D} for human 4-1BB between about 1 M and about 1000 nM, or between about 10 nM to about 500 nM, or between about 20 nM and about 400 nM. As indicated above, the KD may be measured by a number of known methods, for example SPR, as described elsewhere herein. As used in this section, the term “about” means±10% of the value for K_{D} identified in each range. In a related embodiment, the term“about” means±20% of the value for K_{D} as measured by SPR.

The 4-1BB antibody constructs described herein may be prepared, tested and used as described elsewhere herein.

### FRα Antibody Constructs

The present disclosure further provides antibody constructs or antigen-binding fragments thereof that specifically bind to FRα (FRα antibody constructs). These FRα antibody constructs comprise VH and VL sequences as set forth in Tables 17 and 20, and the CDR sequences of these VH and VL sequences can be found in Table 18. In one embodiment, the FRα antibody constructs bind to human FRα.

In some embodiments, the FRα antibody constructs comprise a heavy chain variable sequence comprising three heavy chain CDRs and a light chain comprising three light chain CDRs, wherein the heavy chain CDRs and the light chain CDRs are from antibody 8K22 or antibody 1H06.

In certain embodiments, the FRα antibody constructs comprise VH and VL sequences that are human or humanized. In related embodiments, the FRα antibody construct comprises a VH sequence and a VL sequence having at least 85%, 90%, 95%, 96%, 97%, 98%, or 99% sequence identity to the VH and VL sequences of any one of variants 23794, 23795, 23796, 23797, 23798, 23799, 23800, 23801, 23802, 23803, 23804, 23805, 23806, 23807, 23808, 23809, 23810, 23811, 23812, 23813, 23814, 23815, 23816, 23817, or 23818, derived from the 8K22 antibody.

In other embodiments, the FRα antibody constructs comprise humanized VH and VL sequences comprising the heavy chain CDRs and the light chain CDRs of antibody 1H06.

In some embodiments, humanized FRα antibody constructs comprise a humanized FRα Fab domain that is more stable that than of the parental Fab from which the humanized Fab domain is derived. In related embodiments, the humanized FRα Fab domain can exhibit a Tm that is up to 10° C. higher than that of the parental Fab, In some embodiments, the humanized FRα Fab domain can exhibit a Tm that is up to 5° C. higher than that of the parental Fab.

In certain embodiments, the FRα antibody construct has a binding affinity or K_{D} for human FRα ranging from 100 pM to 100 nM. In some embodiments, the FRα antibody construct has a binding affinity or K_{D} for human FRα ranging from 10 pM to 100 nM. In related embodiments, the FRα antibody construct has a K_{D} for human FRα ranging from 1 nM to 50 nM. In additional related embodiments, the affinity of the FRα antibody constructs for human FRα is measured by Bio-layer interferometry (BLI).

These anti-FRα CDR, VH and VL sequences described in Tables 17, 18, and 20 may be used to construct various formats of antibody constructs as is known in the art. For example, these sequences may be used to construct Fab fragments or scFvs, which may be linked to a scaffold such as an Fc or other scaffold as described herein. Exemplary formats for antibody constructs comprising these CDR, VH and VL sequences are depicted in FIG. 1. The antibody constructs may be monovalent, bivalent or multivalent. In some embodiments, the antibody constructs are monospecific. In some embodiments, the antibody constructs are monospecific and in the naturally occurring format (FSA).

The anti-FRα VH and VL sequences as set forth in Tables 17, 20 and the anti-FRα CDR sequences found in Table 18 may further be used in the construction of hi specific or multispecific antibodies, such as the 4-1BB×FRα antibody constructs described here, or other antibody constructs comprising at least one antigen-binding domain that binds to the FRα.

The FRα antibody constructs described herein may be prepared, tested and used as described elsewhere herein.

Methods of Preparing the Antibody Constructs

The 4-1BB×TAA antibody constructs FRα antibody constructs and 4-1BB antibody constructs described herein may be produced using recombinant methods and compositions, e.g., as described in U.S. Pat. No. 4,816,567. This method and other methods for producing these constructs are described as follows.

Certain embodiments thus relate to one or more nucleic acids encoding an antibody construct described herein. Such nucleic acids may encode the amino acid sequences corresponding to the 4-1BB×TAA antibody constructs or the 4-1BB antibody constructs.

Certain embodiments relate to one or more vectors (e.g., expression vectors) comprising nucleic acid encoding the antibody constructs described herein. In some embodiments, the nucleic acid encoding the antibody construct is included in a multicistronic vector. In other embodiments, each polypeptide chain of the antibody construct is encoded by a separate vector. It is further contemplated that combinations of vectors may comprise nucleic acid encoding a single antibody construct.

Certain embodiments relate to host cells comprising such nucleic acid or one or more vectors comprising the nucleic acid. In some embodiments, for example, where the antibody construct is a multispecific or bispecific antibody, a host cell comprises e.g., has been transformed with): (1) a vector comprising a nucleic acid that encodes an amino acid sequence comprising the VL of the antigen-binding domain and an amino acid sequence comprising the VH of the antigen-binding domain, or (2) a first vector comprising a nucleic acid that encodes an amino acid sequence comprising the VL of the antigen-binding domain and a second vector comprising a nucleic acid that encodes an amino acid sequence comprising the VH of the antigen-binding domain. In some embodiments, the host cell is eukaryotic, e.g. a Chinese Hamster Ovary (CHO) cell, or human embryonic kidney (HEK) cell, or lymphoid cell (e.g., YO, NS0, Sp20 cell).

Certain embodiments relate to a method of making an antibody construct, wherein the method comprises culturing a host cell comprising nucleic acid encoding the antibody construct, as described above, under conditions suitable for expression of the antibody construct, and optionally recovering the antibody construct from the host cell (or host cell culture medium).

For recombinant production of the antibody construct, nucleic acid encoding an antibody construct, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody construct).

The term “substantially purified” refers to a construct described herein, or variant thereof, that may be substantially or essentially free of components that normally accompany or interact with the protein as found in its naturally occurring environment, i.e. a native cell, or host cell in the case of recombinantly produced construct. In certain embodiments, a construct that is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the construct is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the construct is recombinantly produced by the host cells, the protein, in certain embodiments, is present in the culture medium at about 5 g/L, about 4 g/L, about 3 about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells.

In certain embodiments, the term “substantially purified” as applied to a construct comprising a heteromultimer Fc and produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody construct-encoding vectors include prokaryotic or eukaryotic cells described herein,

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, and the like.

As used herein, the term “prokaiyote” refers to prokaryotic organisms. For example, a non-eukaryotic organism can belong to the Eubacteria (including but not limited to, Escherichia coil, Thermus thermophilus, Bacillus stearothermophilus, Pseudomonas fluorescens, Pseudomonas aeruginosa, Pseudomonas putida, and the like) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, and the like) phylogenetic domain,

For example, an antibody construct may be produced in bacteria, in particular when glycosylation and Fc effector function are not needed. For expression of antigen-binding construct fragments and polypeptides in bacteria, see, e.g., U.S. Pat. Nos. 5,648,237, 5,789,199, and 5,840,523. (See also Charlton Methods in Molecular Biology, Vol. 248 (B.K.C. Lo, ed., Humana Press, Totowa, N.J., 2003), pp. 245-254, describing expression of antibody fragments in E. coli.) After expression, the antigen-binding construct may be isolated from the bacterial cell paste in a soluble fraction and can be further purified.

In addition to prokaryotes, eukaryotic microbes such as filamentous fungi or yeast are suitable cloning or expression hosts for Antibody construct-encoding vectors, including fungi and yeast strains whose glycosylation pathways have been “humanized,” resulting in the production of an antigen-binding construct with a partially or fully human glycosylation pattern. See Gerngross, Nat. Biotech. 22:1409-4414 (2004), and Li et al., Nat. Biotech, 24:210-215 (2006).

Suitable host cells for the expression of glycosylated antigen-binding constructs are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antigen-binding constructs in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Viral. 36:59 (1977)); baby hamster kidney cells (BHK); mouse sertoli cells (TM4 cells as described, e.g., in Mather, Biol. Reprod. 23:243-251 (1980)); monkey kidney cells (C V1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻CHO cells (Urlaub et al, Proc. Natl. Acad. Sci. USA 77:4216 (1980)); and myeloma cell lines such as V0, NS0 and Sp2/0. For a review of certain mammalian host cell lines suitable for antigen-binding construct production, see, e.g., Yazaki and Wu Methods in Molecular Biology, Vol. 248 (B.K.C. Lo, ed., Humana Press, Totowa, N.J.), pp. 255-268 (2003).

In some embodiments, the antibody constructs described herein are produced in stable mammalian cells, by a method comprising: transfecting at least one stable mammalian cell with: nucleic acid encoding the antibody construct, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antigen-binding construct in the expressed product.

In some embodiments, in the method of producing an antibody construct in stable mammalian cells, the expression product of the stable mammalian cell comprises a larger percentage of the desired glycosylated antigen-binding construct as compared to the monomeric heavy or light chain polypeptides, or other antibodies.

If required, the antibody constructs can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can used for purification of antigen-binding constructs. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, affinity chromatography if a His-tag is employed, or immobilized anti-flag antibody if a flag-tag is used. For general guidance in suitable purification techniques, see, e.g. incorporated entirely by reference Protein Purification: Principles and Practice, 3^{rd} Ed., Scopes, Springer-Verlag, NY, 1994, incorporated entirely by reference. The degree of purification necessary will vary depending on the use of the antigen-binding constructs. In some instances no purification is necessary.

In certain embodiments, the antibody constructs may be purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAF, Fractogel Q and DEAF columns.

In some embodiments, the antibody constructs are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In some embodiments, the antibody constructs are expressed using cell-free translation or expression systems. Suitable systems are known in the art, such as for example the method described by Stech et al, in Nature Scientific Reports 7:12030, or those described by Gregorio et al, in Methods Protoc. 2019 2:24.

In addition, the antibody constructs can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4-aminobutyric acid, Abu, 2-amino butyric acid, g-Abu, eAhx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as α-methyl amino acids, C α-methyl amino acids, N α-methyl amino acids, and amino acid analogs in general. Furthermore, the amino acid can be D (dextrorotary) or L (levorotary).

### Post-Translational Modifications

In certain embodiments, the antibody constructs described herein are differentially modified during or after translation.

The term “modified,” as used herein, refers to any changes made to a given polypeptide, such as changes to the length of the polypeptide, the amino acid sequence, chemical structure, co-translational modification, or post-translational modification of a polypeptide.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

In some embodiments, the antibody constructs may comprise a modification that is: glycosylation, acetylation, phosphorylation, amidation, derivatization by known protecting/blocking groups, proteolytic cleavage or linkage to an antibody molecule or antigen-binding construct or other cellular ligand, or a combination of these modifications. In some embodiments, the antibody construct is chemically modified by known techniques, including but not limited, to specific chemical cleavage by cyanogen bromide, trypsin, chymotrypsin, papain, V8 protease, NaBH₄; acetylation, formylation, oxidation, reduction; and metabolic synthesis in the presence of tunicamycin.

Additional optional post-translational modifications of antigen-binding constructs include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion f an N-terminal methionine residue as a result of procaryotic host cell expression. The antigen-binding constructs described herein are modified with a detectable label, such as an enzymatic, fluorescent, isotopic or affinity label to allow for detection and isolation of the protein. In certain embodiments, examples of suitable enzyme labels include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

In some embodiments, antigen-binding constructs described herein may be attached to macrocyclic chelators that associate with radiometal ions.

In some embodiments, the antibody constructs described herein may be modified by either natural processes, such as post-translational processing, or by chemical modification techniques which are well known in the art. In certain embodiments, the same type of modification may be present in the same or varying degrees at several sites in a given polypeptide. In certain embodiments, polypeptides from antigen-binding constructs described herein are branched, for example, as a result of ubiquitination, and in some embodiments are cyclic, with or without branching. Cyclic, branched, and branched cyclic polypeptides are a result from posttranslation natural processes or made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company. New York (1993); POST-TRANSLATIONAL COVALENT MODIFICATION OF PROTEINS, B. C. Johnson, Ed., Academic Press, New York, pgs. 1-12 (1983); Sealer et al., Meth. Enzymol. 182:626-646 (1990); Rattan et al., Ann, N.Y. Acad. Sci. 663:48-62 (1992)).

In certain embodiments, antigen-binding constructs described herein may be attached to solid supports, which are particularly useful for immunoassays or purification of polypeptides that are bound by, that bind to, or associate with proteins described herein. Such solid supports include, but are not limited to, glass, cellulose, polyacrylamide, nylon, polystyrene, polyvinyl chloride or polypropylene.

### Additional Optional Modifications

In one embodiment, the antibody constructs described herein can be further modified (i.e., by the covalent attachment of various types of molecules) such that covalent attachment does not interfere with or affect the ability of the 4-1BB×TAA antibody construct to bind to 4-1BB or to the TAA, or affect the ability of the 4-1BB antibody construct to bind to 4-1BB, or negatively affect the stability of these antibody constructs. Similarly, the 4-1BB antibody constructs and FRα antibody constructs may be modified by covalent attachment such that their stability or ability to bind to their target is not significantly affected. Such modifications include, for example, but not by way of limitation, glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formyl anon, metabolic synthesis of tunicamycin, etc.

Certain embodiments contemplate conjugation of the antibody construct to a drug moiety, for example, a toxin, a chemotherapeutic agent, an immune modulator, or a radioisotope. Numerous methods of preparing antibody drug conjugates (ADCs) are known in the art, Examples include methods described in U.S. Pat. No. 8,624,003 (pot method), U.S. Pat. No. 8,163,888 (one-step), and U.S. Pat. No. 5,208,020 (two-step method). See also, Antibody-Drug Conjugates, Series: Methods in Molecular Biology, Laurent Ducty (Ed.), Humana Press, 2013.

The drug moiety of the ADCs is typically a compound or moiety having a cytostatic or cytotoxic effect. In some embodiments, the drug comprised by the ADC is a cytotoxic agent. The term “cytotoxic agent” as used herein refers to a substance that inhibits or prevents the function of cells and/or causes destruction of cells. The term is intended to include radioactive isotopes (for example, ³¹¹At, ¹³¹I, ¹²⁵, ⁹⁰Y, ¹⁸⁶Re, ¹⁸⁸Re, ¹⁵³Sm, ²¹²Bi, ³²P and ¹⁷⁷Lu), chemotherapeutic agents, and toxins such as small molecule toxins or enzymatically active toxins of bacterial, fungal, plant or animal origin, including fragments and/or variants thereof. One skilled in the art will appreciate that some of these categories of drugs overlap and are thus not intended to be mutually exclusive. For example, toxins may also be considered as chemotherapeutic agents in the sense that they are chemical compounds that may be used to treat cancer. In some embodiments, the drug comprised by the ADC is an analog or derivative of a naturally occurring toxin. Examples of such naturally occurring toxins include, but are not limited to, maytansines, auristatins, dolastatins, tubulysins, hemiasterlins, calicheamicins, duocarmycins, pyrrolobenzodiazapenes, amatoxins, camptothecins, Pseudomonas exotoxin (PE), diphtheria toxin (DT), deglycosylated ricin A (dg.A) and gelonin.

In certain embodiments, the drug comprised by the ADCs is a microtubule disrupting agent or a DNA modifying agent. Examples of toxins that are microtubule disrupting agents include, but are not limited to, maytansines, auristatins, dolastatins, tubulysins, hemiasterlins, and analogs and derivatives thereof. Examples of toxins that are DNA modifying agents include, but are not limited to, calicheamicins and other enediyne antibiotics, duocarmycins, pyrrolobenzodiazapenes, amatoxins, camptothecins, and analogs and derivatives thereof.

Exemplary maytansinoids include DM1 (mertansine, emtansine, N₂′-deacetyl-N₂′-(3-mercapto-1-oxopropyl)maytansine), DM3 (N₂′-deacety)-N₂′-(4-mercapto-1-ipxopentyl)maytansine) and DMH4 (ravtansine, soravtansine, N₂′-deacetyl-N₂′-(4-methyl-4-mercapto-1-oxopentyl)maytansine) (see U.S. Patent Application Publication No. US 2009/0202536). Other examples of naturally occurring, synthetic and semi-synthetic maytansinoids are described in Cassady eta)., 2004. Chem. Pharr. Bull., 52(1):1-26, and in U.S. Pat. Nos.4,256,746; 4,361,650; 4,307,016; 4,294,757; 4,424,219; 4,331,598; 4,364,866; 4,313,946; 4,315,929; 4,362,663; 4,322,348 and 4,371,533.

Exemplary dolastatins and auristatins include auristatin E (also known in the art as a derivative of dolastatin-10) and auristatin F, and analogs and derivatives thereof. Auristatin analogs include, for example, esters formed between auristatin E and a keto acid. For example, auristatin E can be reacted with paraacetyl benzoic acid or benzoylvaleric acid to produce auristatin EB (AEB) and auristatin EVB (AEVB), respectively. Other typical auristatins include auristatin F phenylenediamine (AFP), monomethylauristatin F (MMAF) and nionomethylauristatin F (MMAE). The synthesis and structure of exemplary auristatins are described in U.S. Pat. Nos. 6,884,869; 7,098,308; 7,256,257; 7,423,116; 7,498,298 and 7,745.394. Other examples of auristatin analogs, in particular analogs suitable for conjugation via the C-terminus of the drug molecule, include those described in International Publication Nos. WO 2002/088172 and WO 2016/041082.

Exemplary hemiasterlins and herniasterlin analogs and derivatives include those described in International Publication Nos. WO 1996/33211 and WO 2004/026293; U.S. Pat. No. 7,579,323 (which describes the hemiasterlin analog, HTI-286) and International Publication No. WO 2014/144871.

Exemplary calicheamycins and calicheamycin analogs and derivatives include those described in International Publication No. WO 2015/063680, and U.S. Pat. Nos. 5,773,001; 5,714,586 and 5,770,701).

Exemplary duocarmycins and duocarmycin analogs and derivatives include naturally-occurring duocarmycins such as duocarmycins A, B1, B2, C1, C2, D and SA, as well as CC-1065, and analogs and derivatives such as adozelesin, bizelesin and centanamycin. Other calicheamicin analogs and derivatives are described in U.S. Pat. Nos. 4,912,227; 5,070,092; 5,084,468; 5,332,837; 5,641,780; 5,739,350 and 8,889,868.

Exemplary pyrrolobenzodiazapenes (PBD) include various PBD dimers such as those described in U.S. Pat. Nos. 6,884,799; 7,049,311; 7,511,032; 7,528,126; 7,557,099 and 9,056,914, and in International Publication Nos. WO 2007/085930, WO 2009/016516, WO 2011/130598, WO 2011/130613 and WO 2011/130616, and U.S. Patent Application Publication No. US 2011/0256157.

Exemplary amatoxins include a-Amanitin, b-Amanitin, g-Amanitin and e-Amanitin, and analogs and derivatives thereof. Various amatoxins and amatoxin analogues have been described (see, for example, European Patent No. EP 1 859 811 U.S. Pat. No. 9,233,173 and International Publication No. WO 2014/043403).

Exemplary camptothecins (CPT) include irinotecan (CPT-11), SN-38 (7-ethyl-10-hydroxy-camptothecin), 10-hydroxy camptothecin, topotecan, lurtotecan, 9-aminocamptothecin and 9-nitrocamptothecin. Other examples of CPT analogs and derivatives include 7-butyl-10-amino-camptothecin and 7-butyl-9-amino-10,11-methylenedioxy-camptothecin (see U.S. Patent Application Publication No. US 2005/0209263) and aniline containing derivatives of these compounds as described in Burke et al., 2009, Bioconj. Chem. 20(6):1242-1250 and Sharkey et al., 2012. Mol. Cancer Ther. 11:224-234.

In certain embodiments, the drug comprised by the ADC is a chemotherapeutic agent. In some embodiments, the drug comprised by the ADC is an anthracycline, such as doxorubicin, epirubicin, daunorubicin (also known as daunomycin), nemorubicin or an analog or derivative thereof. Derivatization of daunorubicin and doxontbicin for conjugation to antibodies has been described (see, for example, Kratz et al., 2006, Current Med. Chem. 13:477-523, and U.S. Pat. No. 6,630,579).

Additional examples of drugs for use in the ADCs include mTOR inhibitors such as rapamycin (sirolimus) and analogs thereof (“rapalogs”). Rapalogs are considered to be compounds that are structurally related to rapamycin that retain mTOR inhibiting activity and include, for example, esters, ethers, oximes, hydrazones, and hydroxylamines of rapamycin, as well as compounds in which functional groups on the rapamycin core structure have been modified, for example, by reduction or oxidation. Exemplary rapalogs include, but are not limited to, temsirolimus (CC1779), tacrolimus (FK-506), everolimus (RAD001), deforolimus (AP23573), AZD8055 (AstraZeneca), and OSI-027 (OSI).

The selected drug may be conjugated to the antibody construct with or without a linker by any of a variety of methods known in the art. Typically, drugs are conjugated to cysteine or lysine residues in the antibody construct via a linker, which may be cleavable or non-cleavable. Exemplary methods and linkers are provided in Antibody-Drug Coningates, Series: Methods in Molecular Biology, Laurent Ducry (Ed.), Humana Press, 2013.

In some embodiments, the antibody construct may be expressed as fusion proteins comprising a tag to facilitate purification and/or testing etc. As referred to herein, a “tag” is any added series of amino acids which are provided in a protein at either the C-terminus, the N-terminus, or internally that contributes to the identification or purification of the protein. Suitable tags include but are not limited to tags known to those skilled in the art to be useful in purification and/or testing such as albumin binding domain (ABD). His tag, FLAG tag, glutathione-s-transferase, hernagglutinin (HA) and maltose binding protein. Such tagged proteins can also be engineered to comprise a cleavage site, such as a thrombin, enterokinase or factor X cleavage site, for ease of removal of the tag before, during or after purification.

### Methods of Generating Antibodies

If desired, antibodies to a specific antigen of interest may be generated by standard techniques and used as a basis for the preparation of antigen-binding domains of the 4-1BB×TAA antibody constructs, for example for preparing 4-1BB, FRα, NaPi2B, HER2, ormesothelin or LIV1 antigen-binding domains. Briefly, an antibody to an antigen can be prepared by immunizing the purified antigen into rabbits, preparing serum from blood of the rabbits and absorbing the sera to a normal plasma fraction to produce an antibody specific to the antigen. Monoclonal antibody preparations to the antigen may be prepared by injecting with mouse myeloma cells and using the resultant hybridoma cells to produce the monoclonal antibody. Both of these methods are well-known in the art. In some embodiments, antibodies resulting from these methods may be humanized as described elsewhere herein.

As an alternative to humanization, human antibodies can be generated. For example, transgenic animals (e.g., mice) can be used that are capable, upon immunization, of producing a full repertoire of human antibodies in the absence of endogenous immunoglobulin production. See, e.g., Jakobovits et al., 1993, Proc. Natl. Acad. Sci. USA 90:2551; Jakobovits et al 1993, Nature 362:255-258; Bruggermann et al., 1993, Year in Immuno. 7:33; and U.S. Pat. Nos. 5,591,669; 5,589,369; 5,545,807; 6,075,181; 6,150,584; 6,657,103; and 6,713,610.

Alternatively, phage display technology (see, e.g., McCafferty et al., 1990, Nature 348:552-553) can be used to produce human antibodies and antibody fragments in vitro, from immunoglobulin variable (V) domain gene repertoires from unimmunized donors. Phage display can be performed in a variety of formats; for their review see, e.g, Johnson and Chiswell, 1993, Current Opinion in Structural Biology 3:564-571. Human antibodies may also be generated by in vitro activated B cells (see U.S. Pat. Nos. 5,567,610 and 5,229,275). Novel antibody sequences may also be generated de novo using platforms such as the HuTarg™ platform (Innovative Targeting Solutions Inc Vancouver Canada).

The affinity of antibodies for an antigen may be altered according to methods known in the art.

### Testing the Antibody Constructs

The ability of the 4-1BB×TAA antibody constructs to bind to 4-1BB and the TAA can be tested according to methods known in the art including antigen-binding assays or cell binding assays. Antigen-binding assays are carried out by incubating the antibody construct with antigen (4-1E113 or TAA), either purified, or in a mixture and assessing the amount of 4-1BB×TAA antibody construct bound to the antigen, compared to controls. The amount of 4-1BB×TAA antibody construct bound to the antigen can by assessed by ELISA, or SPR (surface plasmon resonance), for example. Cell binding assays are carried out by incubating the antibody construct with cells that express 4-1BB or the TAA of interest (such cells are commercially available). The amount of 4-1BB×TAA antibody construct bound to the cells can be assessed by flow cytometry, for example, and compared to binding observed in the presence of controls. Methods for carrying out these types of assays are well known in the art. Similar methods may be used to assess the ability of 4-1BB antibody constructs to bind to 4-1BB. Likewise, the ability of FRα antibody constructs to bind to purified FRα or FRα expressed on cells may be determined.

The 4-1BB×TAA antibody constructs or 4-1BB antibody constructs may also be tested to determine if they promote activation of cells expressing 4-1BB. Suitable assays include the co-culture assays described in the examples, such as the NR-κB-luciferase/4-1BB-expressing Jurkat cell assay, or the primary T cell co-culture assay. TAA expressing cell lines suitable for use in these assays are readily identified by one of skill in the art. For example, to assess the ability of a 4-1BB×FRα antibody construct to promote activation of 4-1BB in the presence of cells expressing FRα, a number of cell line may be used, for example but not limited to IGROV1, OVCAR3, OVKATE, NCI-H441, NCI-H661, NCI-H1975, or HCC827. These FRα-expressing cell lines can be divided into FRα^{high}, FRα^{mid} and FRα^{low} based on numbers of receptor expressed in these cells as measured by binding of a reference antibody to these cells via, for example, quantitative flow cytometry experiments. In some embodiments, FRα^{high} cells may express greater than about 500,000 FRα molecules per cell, FRα^{mid} between about 200,00( )and about 500,000 FRα molecules per cell and FRα^{low} below about 200,00FRα molecules per cell. In some embodiments, FRα^{neg} cells are those where binding of a reference antibody to FRα cannot be detected by flow cytometry.

In vivo efficacy of the 4-1BB×TAA antibody constructs, 4-1BB antibody constructs, or FRα antibody constructs may also be evaluated by standard techniques. For example, the effect of the antibody constructs on tumor growth can be examined in various tumor models. Several suitable animal models are known in the art to test the ability of candidate therapies to treat cancers, such as, for example, breast cancers or gastric cancers. Some models are commercially available. Suitable models include syngeneic or xenograft models (see below). The construct to be tested is generally administered after the tumor has been established in the animal, but in some cases, the construct can be administered with the cell line. The volume of the tumor, survival of the animal and/or a response which may correlate with function is monitored in order to determine if the construct is able to treat the tumor. The construct may be administered intravenously (i.v.), intraperitoneally (i.p.) or subcutaneously (s.c.). Dosing schedules and amounts vary but can be readily determined by the skilled person. An exemplary dosage would be 10 mg/kg once weekly. Tumor growth can be monitored by standard procedures. For example, when labelled tumor cells have been used, tumor growth may be monitored by appropriate imaging techniques. For solid tumors, tumor size may also be measured by caliper. Other responses which may be indicative of efficacy of the construct may include increases or decreases in systemic or localized cytokine or chemokine responses (such as but not limited to IFNγ, IL-2, TNFα, CXCL8, IP-10, RANTES), increases or decreases in the number of immune cells (such as NK, NKT, B, DCs, Macrophages, Neutrophils), increase or decrease of the expression of key surface, intracellular or nuclear proteins on or in either the immune cells (such as but not limited to PD1, Tim3, Lag3, 4-1BB, CD163, EOMeS, TOX) or on the surface of the tumour (PDL1). It is further contemplated that these responses can also be assessed in in vitro assays such as the immune cell co-culture assays described herein and in the Examples in order to test the activity of candidate 4-1BB×TAA antibody constructs.

In vivo mouse tumour models may be syngeneic or xenograft models. Syngeneic models involve grafting a tumour from one mouse to another where the genetic background of the two mice is sufficiently close that the recipient mouse immune system does not reject the tumour (Teicher, B A Tumor models in cancer research, Springer 2011). This can be done either directly from mouse to mouse, or via a cell line that is stable in culture. The cell lines can be engineered using standard molecular biology techniques to express the TAA if they do not naturally, which enables control over expression levels.

Xenograft tumour models involve the grafting of a tumour from another species (usually human) into a mouse. The mouse would normally reject the tumour as non-self, but is engineered to lack a functional adaptive immune system by a set of mutations which prevent T, B and NK cell development and impair myeloid cell function. Common strains of mice which are suitable for engraftment of human tumour cells are NSG™, NOG™ and NRG mice, which have combined either Prkdc^{scid} or Rag1^{-/-} with IL2rg mutations on a NOD background (Morton et al, Cancer Research 2016:76:21 pp61.53-6158). These mice can be implanted with human tumours, but lack an adaptive immune system. The human tumour cells can come from cells which are stable in cell culture (such as OVCAR3, HCC827, IGROV1 or H1975) or from patients which have had their tumours removed. The immune system can be then be recapitulated by addition of either PBMC, I cells or CD34⁺ HSC from human donors. These immune cells can then reconstitute the host with T cells to act as effectors during an experiment.

### Competitive Binding Analyses and Epitope Mapping of Antibody Sequences

The 4-1BB epitopes bound by the 4-1BB antibody constructs or the epitopes bound by the FRα antibody constructs described herein can be determined by standard competitive binding analysis (Fendly et al, Cancer Research 50: 1550-1558 (1990)). For example, for 4-1BB, cross-blocking studies may be done on antibodies by direct fluorescence on intact cells engineered to express 4-1BB using suitable methods to quantitate fluorescence. Each test antibody is conjugated with fluorescein isothiocyanate (FITC), using established procedures (Wofsy et al, Selected Methods in Cellular Immunology, p. 287, Mishel and Schiigi (eds.) San Francisco: W.J. Freeman Co. (1980)). Antibodies are considered to share an epitope if each blocked binding of the other by 40% or greater in comparison to an irrelevant antibody control and at the same antibody concentration. Using this assay, one of ordinary skill in the art can identify other antibodies that bind to the same epitope as those described herein. Deletion analysis may be conducted to identify the approximate location in the polypeptide sequence of 4-1BB of the antigenic epitopes. In a similar manner, the epitopes bound by the TAA antigen-binding domains of the 4-1BB×TAA antibody construct, or the FRα antibody construct may also be identified.

### Pharmaceutical Compositions

Certain embodiments relate to pharmaceutical compositions comprising an antibody construct described herein and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, vehicle, or combination thereof, with which the construct is administered. Such pharmaceutical carriers can be sterile liquids, such as water and oils, including those of petroleum, animal, vegetable or synthetic origin, such as peanut oil, soybean oil, mineral oil, sesame oil and the like. In some aspects, the carrier is a man-made carrier not found in nature. Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical carriers are described in “Remington's Pharmaceutical Sciences” by E. W. Martin.

The pharmaceutical compositions may be in the form of solutions, suspensions, emulsion, tablets, pills, capsules, powders, sustained-release formulations and the like. The composition may be formulated as a suppository, with traditional binders and carriers such as triglycerides. Oral formulations may include standard carriers such as pharmaceutical grades of mannitol, lactose, starch, magnesium stearate, sodium saccharine, cellulose, magnesium carbonate, and the like.

Pharmaceutical compositions will contain a therapeutically effective amount of the antibody construct, together with a suitable amount of carrier so as to provide the form for proper administration to a patient. The formulation should suit the mode of administration.

In certain embodiments, the composition comprising the antibody construct is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, and the like.

### Methods of Using the Antibody Constructs

In certain embodiments, provided is a method of treating a cancer comprising administering to a subject in which such treatment, prevention or amelioration is desired, a 4-1BB×TAA antibody construct described herein, in an amount effective to treat or ameliorate the cancer. In other embodiments, there is provided a method of using the 4-1BB×TAA antibody construct in the preparation of a medicament for the treatment, prevention, or amelioration of cancer in a subject. In additional embodiments, the 4-1BB×TAA antibody constructs may be used for the treatment of cancer in a subject in need thereof. The 4-1BB antibody construct and FRα antibody constructs may also be used in the treatment of cancer as described below.

In some embodiments, the 4-1BB×TAA antibody construct, 4-1BB antibody construct, or FRα antibody construct may be used in a subject to treat, prevent or ameliorate a cancer selected from breast cancer, bladder cancer, colorectal cancer, head and neck cancer, liver cancer, lung cancer, ovarian cancer, pancreatic cancer, skin cancer, prostate cancer, kidney cancer or thyroid cancer. In specific embodiments, the 4-1BB×TAA antibody construct may be used to treat, prevent or ameliorate lung or ovarian cancers in a subject. In certain embodiments, the 4-1BB×TAA antibody construct may be used in the treatment of solid tumors, in embodiments where the 4-1BB×TAA is a 4-1BB×FRα antibody construct, the construct may be used to treat, prevent or ameliorate lung or ovarian cancers in a subject. In embodiments where the 4-1BB×TAA is a 4-1BB×NaPi2b antibody construct, the construct may be used to treat, prevent or ameliorate lung cancer in a subject.

The term “subject” refers to an animal, in some embodiments a mammal, which is the object of treatment, observation or experiment. An animal may be a human, a non-human primate, a companion animal (e.g., dogs, cats, and the like), farm animal (e.g., cows, sheep, pigs, horses, and the like) or a laboratory animal (e.g., rats, mice, guinea pigs, and the like).

The term “mammal” as used herein includes but is not limited to humans, non-human primates, canines, felines, murines, bovines, equines, and porcines.

“Treatment” or “treat” refers to clinical intervention in an attempt to alter the natural course of the individual or cell being treated, and can be performed during the course of clinical pathology. Desirable effects of treatment include preventing recurrence of disease, alleviation of symptoms, diminishing of any direct or indirect pathological consequences of the disease, preventing metastasis, decreasing the rate of disease progression, amelioration or palliation of the disease state, and remission or improved prognosis. In some embodiments, the 4-1BB×TAA antibody constructs described herein are used to delay development of a disease or disorder in a subject. In one embodiment, 4-1BB×TAA antibody constructs and methods described herein effect tumor regression in a subject. In one embodiment, the 4-1BB×TAA antibody constructs and methods described herein effect inhibition of tumor/cancer growth in a subject.

The term “effective amount” as used herein refers to that amount of antibody construct being administered, which will accomplish the goal of the recited method, e.g., relieve to some extent one or more of the symptoms of the disease, condition or disorder being treated. The amount of the composition described herein which will be effective in the treatment, inhibition and prevention of a disease or disorder associated with aberrant expression and/or activity of a therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

“Therapeutically effective amount,” as used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

The 4-1BB×TAA antibody construct can be administered to a subject. Various delivery systems are known and can be used to administer an antibody construct formulation described herein, e.g., encapsulation in liposomes, microparticles, microcapsules, recombinant cells capable of expressing the compound, receptor-mediated endocytosis (see, e.g., Wu and Wu, J. Biol. Chem. 262:44294432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the antibody construct compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the antibody constructs, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody construct, described herein, care must be taken to use materials to which the protein does not absorb.

In another embodiment, the antibody constructs or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the antibody constructs or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, vol. 2, pp. 115-138 (1984)).

In a specific embodiment comprising a nucleic acid encoding the antibody constructs described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The amount of the antibody construct which will be effective in the treatment, inhibition or prevention of a disease or disorder can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

The antibody constructs described herein may be administered alone or in combination with other alternate forms of treatments or anti-cancer therapy (e.g., radiation therapy, chemotherapy, hormonal therapy, immunotherapy, bi specific antibodies, and anti-tumor agents). Generally, administration of products of a species origin or species reactivity (in the case of antibodies) that is the same species as that of the patient is preferred.

In some embodiments, the antibody construct may be used in the treatment of a patient who has undergone one or more alternate forms of anti-cancer therapy. In some embodiments, the patient has relapsed or failed to respond to one or more alternate forms of anti-cancer therapy, In other embodiments, the antibody construct is administered to a patient in combination with one or more alternate forms of anti-cancer therapy. In other embodiments, the antibody construct is administered to a patient that has become refractory to treatment with one or more alternate forms of anti-cancer therapy.

### Kits and Articles of Manufacture

Also described herein are kits comprising one or more antibody constructs. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the antibody construct.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

Certain embodiments relate to an article of manufacture containing materials useful for treatment of a patient as described herein. The article of manufacture comprises a container and a label or package insert on or associated with the container. Suitable containers include, for example, bottles, vials, syringes, intravenous solution bags, etc. The containers may be formed from a variety of materials such as glass or plastic. The container holds a composition comprising the antibody construct which is by itself or combined with another composition effective for treating the patient and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises an antibody construct described herein; and (b) a second container with a composition contained therein, wherein the composition in the second container comprises a further cytotoxic or otherwise therapeutic agent. In such embodiments, the article of manufacture may further comprise a package insert indicating that the compositions can be used to treat a particular condition, Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. The article of manufacture may optionally further include other materials desirable from a commercial and user standpoint, including other buffers, diluents, filters, needles, and syringes.

### Polypeptides and Polynucleotides

As described herein, the antibody constructs comprise at least one polypeptide. Certain embodiments relate to polynucleotides encoding such polypeptides described herein.

The antibody constructs, polypeptides and polynucleotides described herein are typically isolated. As used herein, “isolated” means an agent (e.g., a polypeptide or polynucleotide) that has been identified and separated and/or recovered from a component of its natural cell culture environment. Contaminant components of its natural environment are materials that would interfere with diagnostic or therapeutic uses for the antibody construct, and may include enzymes, hormones, and other proteinaceous or non-proteinaceaous solutes. Isolated also refers to an agent that has been synthetically produced, e.g., via human intervention.

The terms “polypeptide,” “peptide” and “protein” are used interchangeably herein to refer to a polymer of amino acid residues. That is, a description directed to a poly peptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full-length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the antibody constructs described herein may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Also included herein are polynucleotides encoding polypeptides of the antibody constructs. The term “polynucleotide” or “nucleotide sequence” is intended to indicate a consecutive stretch of two or more nucleotide molecules. The nucleotide sequence may be of genomic, cDNA, RNA, semisynthetic or synthetic origin, or any combination thereof.

The term “nucleotide sequence” or “nucleic acid sequence” is intended to indicate a consecutive stretch of two or more nucleotide molecules. The nucleotide sequence can be of genomic, cDNA, RNA, semisynthetic or synthetic origin, or any combination thereof.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing a nucleic acid sequence into a cell.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol, Chem. 260:2605-2608 (1985); Rossolini et al., Mol. Cell. Probes 8:91-98 (1994)).

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also encompasses every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid.

Conservative substitution tables providing functionally similar amino acids are known to those of ordinary skill in the art. Such conservatively modified variants are in addition to and do not exclude polymorphic variants, interspecies homologs, and alleles described herein. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139] 8) Cysteine (C), r′vlethionine (M) (see, e.g., Creighton, Proteins: Structures and Molecular Properties (W H Freeman & Co.; 2nd edition (December 1993).

The term “identical” in the context of two or more nucleic acids or polypeptide sequences, refers to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75100 amino acids or nucleotides in length, or, where not specified, across the entire sequence of a polynucleotide or polypeptide. A polynucleotide encoding a polypeptide described herein, including homologs from species other than human, may be obtained by a process comprising the steps of screening a library under stringent hybridization conditions with a labeled probe having a polynucleotide sequence described herein or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP. BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res, 25:3389-3402, and Altschul et al, (1990) J. Mol. Biol. 215:403-410, respectively. Software for performing BLAST analyses is publicly available through the National Center for Biotechnology Information available at the World Wide Web at nebi.nlm.nih.gov. The BLAST algorithm parameters W, T, and X determine the sensitivity and speed of the alignment. The BLASTN program (for nucleotide sequences) uses as defaults a wordlength (W) of 11, an expectation (F) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

As used herein, the term “engineer,” and grammatical variations thereof is considered to include any manipulation of a peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. The engineered proteins are expressed and produced by standard molecular biology techniques.

A derivative, or a variant of a polypeptide is said to share “homology” or be “homologous” with the polypeptide if the amino acid sequences of the derivative or variant has at least 50% identity with a 100 amino acid sequence from the original polypeptide. In certain embodiments, the derivative or variant is at least 75% the same as that of either the polypeptide or a fragment of the polypeptide having the same number of amino acid residues as the derivative. In various embodiments, the derivative or variant is at least 85%, 90%, 95% or 99% the same as that of either the polypeptide or a fragment of the polypeptide having the same number of amino acid residues as the derivative.

In some aspects, an antibody construct comprises an amino acid sequence that is at least 80, 85, 90, 91, 92, 93, 94, 95, 96, 97, 98, 99, or 100% identical to a relevant amino acid sequence or fragment thereof set forth in the Tables or Accession Numbers disclosed herein. In some aspects, an isolated antibody construct comprises an amino acid sequence encoded by a polynucleotide that is at least 80, 85, 90, 91, 92, 93, 94, 95, 96, 97, 98, 99, or 100% identical to a relevant nucleotide sequence or fragment thereof set forth in Tables or Accession Numbers disclosed herein.

### Embodiments

A1. An antibody construct comprising:
   a) a first 4-1BB-binding domain that binds to a 4-1BB extracellular domain (4-1BB ECD), and
   b) a tumor-associated antigen (TAA) antigen binding domain (TAA antigen-binding domain) that binds to a TAA.
      wherein the first 4-1BB-binding domain and the TAA antigen-binding domain are linked directly or indirectly to a scaffold.
A2. The antibody construct according to embodiment A1, wherein the first 4-1BB binding domain is a first 4-1BB antigen-binding domain or a 4-1BB ligand.
A3. The antibody construct according to embodiment A2, wherein the first 4-1BB antigen-binding domain binds to a first epitope of 4-1BB ECD.
A4. The antibody construct according to any one of embodiments A1 to A3, further comprising a second 4-1BB binding domain.
A5. The antibody construct according to embodiment A4, wherein the second 4-1BB binding domain is a second 4-1BB antigen-binding domain.
A6. The antibody construct according to embodiment A5, wherein the second 4-1BB antigen-binding domain binds to a second epitope of 4-1BB ECD.
A7. The antibody construct according to embodiment A5 or A6, wherein the first epitope of 4-1BB ECD is the same as the second epitope of 4-1BB ECD.
A8. The antibody construct according to embodiment A5 or A6, wherein the first epitope of 4-1BB ECD is different from the second epitope of 4-1.BB ECD.
A9. The antibody construct according to any one of embodiments A4 to A8, wherein the first or second 4-1BB antigen-binding domain binds to human and cynomolgus 4-1BB.
A10. The antibody construct according to any one of embodiments A4 to A9, wherein the 4-1BB antigen-binding domain binds to domain 1 or domain 2 of 4-1BB.
A11. The antibody construct according to any one of embodiments A4 to A9, wherein the 4-1BB antigen-binding domain binds to other than domains 3 and 4 or 4-1BB.
A12. The antibody construct according to any one of embodiments A1 to A9, wherein the first 4-1BB-binding domain is a first 4-1BB antigen-binding domain comprising a heavy chain variable sequence comprising three CDRs and light: chain variable sequence comprising three CDRs and the heavy chain variable sequence and the light chain variable sequence is from any one of variants v28726, v28727, v28728, v28730, v20022, v28700, v28704, v28705, v28706, v28711, v28712, v28713, v20036, v28696, v28697, v28698, v28701, v28702, v28703, v28707, v20023, v28683, v28684, v28685, v28686, v28687, v28688, v28689, v28690, v28691, v28692, v28694, or v28995.
A13, The antibody construct according to any one of embodiments A4 to A12, wherein the first 4-1BB antigen-binding domain and/or the second 4-1BB antigen-binding domain are in a Fab format.
A14. The antibody construct according to any one of embodiments A4 to A12, wherein one of the first 4-1BB antigen-binding domain or the second 4-1BB antigen-binding domain is in an scFv format.
A15. The antibody construct according to any one of embodiments A1 to A14, wherein the TAA antigen-binding domain is a folate receptor-α (FRα) antigen-binding domain, a Solute Carrier Family 34 Member 2 (NaPi2b) antigen-binding domain, a HER2 antigen-binding domain, a mesothelin antigen-binding domain, or a Solute Carrier Family 39 Member 6 (LIV-1) antigen-binding domain.
A16, The antibody construct according to any one of embodiments A1 to A15 wherein the TAA antigen-binding domain is a Ma antigen-binding domain.
A17, The antibody construct according to embodiment A16, wherein the FRα antigen-binding domain comprises the three heavy chain CDRs and the three light chain CDRs of antibody 8K22 or 1H06.
A18. The antibody construct according to embodiment A17, wherein the FRα antigen-binding domain is a human or humanized antigen-binding domain.
A19. The antibody construct according to any one of embodiments A1 to A18, wherein the TAA antigen-binding domain is in an scFv format.
A20. The antibody construct according to any one of embodiments A1 to A18, wherein the TAA antigen-binding domain is in a Fab format.
A21. The antibody construct according to any one of embodiments A1 to A20, wherein the scaffold is a dimeric Fc construct having a first Fc polypeptide and a second Fc polypeptide, each Fc polypeptide comprising a CH3 sequence, or wherein the scaffold is a linker or an albumin polypeptide.
A22. The antibody construct according to embodiment A21, wherein the scaffold is a heterodimeric Fc construct having a first Fc polypeptide that is different fr©m the second Fc polypeptide, and wherein the CH3 sequences of the first Fc polypeptide and the second. Fc polypeptide comprise amino acid substitutions that promote the formation of a heterodimeric Fc.
A23. The antibody construct according to embodiment A22, wherein:
   a) one Fc polypeptide comprises the amino acid substitutions T350V_L351Y_F405A_Y407V and the other Fc polypeptide comprises the amino acid substitutions T350V_T366L_K392L_T394W;
   b) one Fc polypeptide comprises the amino acid substitutions T350V_T366L_K392M_T394W and the other Fc polypeptide comprises the amino acid substitutions T350V_L351Y_F405A_Y407V;
   c) one Fc polypeptide comprises the amino acid substitutions L351Y_F405A_Y407V and the other Fc polypeptide comprises the amino acid substitutions T366L_K392M_T394W;
   d) one Fc polypeptide comprises the amino acid substitutions L351Y_F405A_Y407V and the other Fc polypeptide comprises the amino acid substitutions T366L_K392L_T394W; or
   e) one Fc polypeptide comprises the amino acid substitutions L351Y_S400E_F405A_Y407V and the other Fc polypeptide comprises the amino acid substitutions T366I_N390R_K392M_T394W,
   wherein the numbering of residues is according to the EU numbering system.
A24. The antibody construct according to any one of embodiments A21 to A23, further comprising one or more amino acid modifications that reduce effector function.
A25. The antibody construct according to any one of embodiments A21 to A24, wherein the first 4-1BB antigen-binding domain is linked to the N terminus of the first Fc polypeptide, and the TAA antigen-binding domain is linked to the C terminus of the first Fc polypeptide.
A26. The antibody construct according to embodiment A25, further comprising a second 4-1BB antigen-binding domain is linked to the N terminus of the second Fc polypeptide.
A27. An antibody construct or antigen-binding fragment thereof, that specifically binds to 4-1BB, comprising: a heavy chain variable sequence comprising three CDRs and light chain variable sequence comprising three CDRs and the heavy chain variable sequence and the light chain variable sequence is from any one of variants v28726, v28727, v28728, v28730, v20022, v28700, v28704, v28705, v28706, v28711, v28712, v28713, v20036, v28696, v28697, v28698, v28701, v28702, v28703, v28707, v20023, v28683, v28684, v28685, v28686, v28687, v28688, v28689, v28690, v28691, v28692, v28694, or v28995.
A28. The antibody construct according to embodiment A27, comprising a VH sequence and a VL sequence having at least 85% sequence identity to the VH and VL sequences of any one of variants v28726, v28727, v28728, v28730, v20022, v28700, v28704, v28705, v28706, v28711, v28712, v28713, v20036, v28696, v28697, v28698, v28701, v28702, v28703, v28707, v20023, v28683, v28684, v28685, v28686, v28687, v28688, v28689, v28690, v28691, v28692, v28694, or v28995.
A29. The antibody construct according to any one of embodiments A1 to A28, conjugated to a drug.
A30, A pharmaceutical composition comprising the antibody construct of any one of embodiments A1 to A29.
A31. One or more nucleic acids encoding the antibody construct according to any one of embodiments A1 to A28.
A32. One or more vectors comprising the one or more nucleic acids according to embodiment A31.
A33, An isolated cell comprising the one or more nucleic acid according to embodiment A31, or the one or more vectors according to embodiment A32.
A34. A method of preparing the antibody construct according to any one of embodiments A1 to A29, comprising culturing the isolated cell of embodiment A33 under conditions suitable for expressing the antibody construct, and purifying the antibody construct.
A35. A method of treating a subject having a cancer, comprising administering to the subject an effective amount of the antibody construct according to any one of embodiments A1 to A29.
A36, Use of an effective amount of the antibody construct according to any one of embodiments A1 to A29 for the treatment of cancer in a subject in need thereof.
A37. Use of the antibody construct according to any one of embodiments A1 to A29 in the preparation of a medicament for the treatment of cancer.
A38. The antibody construct according to any one of embodiments A1 to A29, for use in the treatment of cancer in a subject.
B1. An antibody construct comprising:

a) a first 4-1BB-binding domain that binds to a 4-1BB extracellular domain (4-1BB ECD), and
b) a first tumor-associated antigen (TAA) antigen binding domain (TAA antigen-binding domain) that binds to a TAA,
   wherein the first 4-MB-binding domain and the first TAA antigen-binding domain are linked directly or indirectly to a scaffold.
B2. The antibody construct according to embodiment B1, wherein the first 4-1BB binding domain is a first 4-1BB antigen-binding domain.
B3. The construct according to embodiment B1 or B2, wherein the first 4-1BB antigen-binding domain is derived from an agonistic anti-4-1BB antibody.
B4. The construct according to any one of embodiments B 1 to B3, wherein:
   a) the first 4-1BB antigen-binding domain in monovalent form has an KD for human 4-1BB between about 1 μM and 100 pM; and/or
   b) the 4-1BB×TAA antibody construct binds to one or more TAA-expressing cell lines as determined by flow cytometry; and/or
   c) the 4-1BB×TAA antibody construct binds to human 4-1BB as measured by SPR and binds to the TAA as measured by SPR; and/or
   d) the 4-1BB×TAA antibody construct stimulates 4-1BB activity in I cells as measured by cytokine production, in the presence of TAA expressing cells; and/or
   e) the 4-1BB×TAA antibody construct binds to 4-1BB-expressing cells and binds to TAA-expressing cells as measured by flow cytometry; and/or
   f) the 4-1BB×TAA antibody constructs are capable of stimulating 4-1BB signalling in 4-1BB-expressing cells in the presence of TAA-expressing cells.
B5, The antibody construct according to any one of embodiments B1 to B4, wherein the first 4-1BB antigen-binding domain comprises: a) a heavy chain variable domain comprising the three heavy chain CDRs of antibody 1C3, and a light chain variable domain comprising the three light chain CDRs of antibody 1C3; b) a heavy chain variable domain comprising the three heavy chain CDRs of antibody 1C8, and a light chain variable domain comprising the three light chain CDRs of antibody 1C8; c) a heavy chain variable domain comprising the three heavy chain CDRs of antibody 1G1, and a light chain variable domain comprising the three light chain CDRs of antibody 1G1; d) a heavy chain variable domain comprising the three heavy chain CDRs of antibody 2E8, and a light chain variable domain comprising the three light chain CDRs of antibody 2E8; e) a heavy chain variable domain comprising the three heavy chain CDRs of antibody 3E7, and a light chain variable domain comprising the three light chain CDRs of antibody 3E7; f) a heavy chain variable domain comprising the three heavy chain CDRs of antibody 4E6, and a light chain variable domain comprising the three light chain CDRs of antibody 4E6; g) a heavy chain variable domain comprising the three heavy chain CDRs of antibody 5G8, and a light chain variable domain comprising the three light chain CDRs of antibody 5G8; or h) a heavy chain variable domain comprising the three heavy chain CDRs of antibody 6B3, and a light chain variable domain comprising the three light chain CDRs of antibody 6B3.
B6. The antibody construct according to any one of embodiments B 1 to B5, wherein the first 4-1BB antigen-binding domain is a human or humanized antigen-binding domain.
B7. The antibody construct according to embodiment B6, wherein the first 4-1BB antigen-binding domain comprises:
a) a heavy chain variable domain (VH) sequence that is at least 85% identical to the VH sequence of v28726 and a light chain variable domain (VL) sequence that is at least 85% identical to the VL sequence of v28726;
b) a heavy chain variable domain (VH) sequence that is at least 85% identical to the VH sequence of v28727 and a light chain variable domain (VL) sequence that is at least 85% identical to the VL sequence of v28727;
c) a heavy chain variable domain (VH) sequence that is at least 85% identical to the VH sequence of v28728 and a light chain variable domain (VL) sequence that is at least 85% identical to the VL sequence of v28728;
d) a heavy chain variable domain (VH) sequence that is at least 85% identical to the VH sequence of v28730 and a light chain variable domain (VL) sequence that is at least 85% identical to the VL sequence of v28730;
e) a heavy chain variable domain (VH) sequence that is at least 85% identical to the VH sequence of v28700 and a light chain variable domain (VL) sequence that is at least 85% identical to the VL sequence of v28700;
f) a heavy chain variable domain (VH) sequence that is at least 85% identical to the VH sequence of v28704 and a light chain variable domain (VL) sequence that is at least 85% identical to the VL sequence of v28704;
g) a heavy chain variable domain (VH) sequence that is at least 85% identical to the VH sequence of v28705 and a light chain variable domain (VL) sequence that is at least 85% identical to the VL sequence of v28705;
h) a heavy chain variable domain (VH) sequence that is at least 85% identical to the VH sequence of v28706 and a light chain variable domain (VL) sequence that is at least 85% identical to the VL sequence of v28706;
i) a heavy chain variable domain (VH) sequence that is at least 85% identical to the VH sequence of v28711 and a light chain variable domain (VL) sequence that is at least 8590 identical to the VL sequence of v28711;
j) a heavy chain variable domain (VH) sequence that is at least 85% identical to the VH sequence of v28712 and a light chain variable domain (VL) sequence that is at least 85% identical to the VL sequence of v28712;
k) a heavy chain variable domain (VH) sequence that is at least 85% identical to the VH sequence of v28713 and a light chain variable domain (VL) sequence that is at least 85% identical to the VL sequence of v28713;
l) a heavy chain variable domain (VH) sequence that is at least 85% identical to the VH sequence of v28696 and a light chain variable domain (VL) sequence that is at least 85% identical to the VL sequence of v28696;
m) a heavy chain variable domain (VH) sequence that is at least 85% identical to the VH sequence of v28697 and a light chain variable domain (VL) sequence that is at least 85% identical to the VL sequence of v28697;
n) a heavy chain variable domain (VH) sequence that is at least 85% identical to the VH sequence of v28698 and a light chain variable domain (VL) sequence that is at least 85% identical to the VI: sequence of v28698;
o) a heavy chain variable domain (VH) sequence that is at least 85% identical to the VH sequence of v28701 and a light chain variable domain (VL) sequence that is at least 85% identical to the VL sequence of v28701;
p) a heavy chain variable domain (VH) sequence that is at least 85% identical to the VH sequence of v28702 and a light chain variable domain (VL) sequence that is at least 85% identical to the VL sequence of v28702;
q) a heavy chain variable domain (VH) sequence that is at least 85% identical to the VH sequence of v28703 and a light chain variable domain (VL) sequence that is at least 85% identical to the VL sequence of v28703;
r) a heavy chain variable domain (VH) sequence that is at least 85% identical to the VH sequence of v28707 and a light chain variable domain (VL) sequence that is at least 85% identical to the VL sequence of v28707;
s) a heavy chain variable domain (VH) sequence that is at least 85% identical to the VH sequence of v28683 and a light chain variable domain (VL) sequence that is at least 85% identical to the VL sequence of v28683;
t) a heavy chain variable domain (VH) sequence that is at least 85% identical to the VH sequence of v28684 and a light chain variable domain (VL) sequence that is at least 85% identical to the VL sequence of v28684;
u) a heavy chain variable domain (VH) sequence that is at least 85% identical to the VH sequence of v28685 and a light chain variable domain (VL) sequence that is at least 85% identical to the VL sequence of v28685;
v) a heavy chain variable domain (VH) sequence that is at least 85% identical to the VH sequence of v28686 and a light chain variable domain (VL) sequence that is at least 85% identical to the VL sequence of v28686;
w) a heavy chain variable domain (VH) sequence that is at least 85% identical to the VH sequence of v28687 and a light chain variable domain (VL) sequence that is at least 85% identical to the VL sequence of v28687;
x) a heavy chain variable domain (VH) sequence that is at least 85% identical to the VH sequence of v28688 and a light chain variable domain (VL) sequence that is at least 85% identical to the VL sequence of v28688;
y) a heavy chain variable domain (VH) sequence that is at least 85% identical to the VH sequence of v28689 and a light chain variable domain (VL) sequence that is at least 85% identical to the VL sequence of v28689;
z) a heavy chain variable domain (VH) sequence that is at least 85% identical to the VH sequence of v2869( )and a light chain variable domain (VL) sequence that is at least 85% identical to the VL sequence of v28690;
aa) a heavy chain variable domain (VH) sequence that is at least 85% identical to the VH sequence of v28691 and a light chain variable domain (VL) sequence that is at least 85% identical to the VL sequence of v28691;
ab) a heavy chain variable domain (VH) sequence that is at least 85% identical to the VH sequence of v28692 and a light chain variable domain (VL) sequence that is at least 85% identical to the VL sequence of v28692;
ac) a heavy chain variable domain (VH) sequence that is at least 85% identical to the VH sequence of v28694 and a light chain variable domain (VL) sequence that is at least 85% identical to the VL sequence of v28694; or
ad) a heavy chain variable domain (VH) sequence that is at least 85% identical to the VH sequence of v28695 and a light chain variable domain (VL) sequence that is at least 85% identical to the VL sequence of v28695.
B8. The antibody construct according to any one of embodiments B1 to B7, further comprising a second 4-1BB binding domain.
B9. The antibody construct according to any one of embodiments B1 to B8, wherein the second 4-1BB binding domain is a second 4-1BB antigen-binding domain.
B10. The antibody construct according to embodiment B9, wherein the second 4-1BB antigen-binding domain is the same as the first 4-1BB antigen-binding domain.
B11. The antibody construct according to embodiment B10, wherein the first 4-1BB antigen-binding domain and/or the second 4-1BB antigen-binding domain are in a Fab format.

B12. The antibody construct according to any one of embodiments B1 to B11, wherein the TAA antigen-binding domain is a folate receptor-α (FRα) antigen-binding domain, a Solute Carrier Family 34 Member 2 (NaPi2b) antigen-binding domain, a HER2 antigen-binding domain, a mesothelin antigen-binding domain, or a Solute Carrier Family 39 Member 6 (LIV-1) antigen-binding domain.
B13. The antibody construct according to any one of embodiments B1 to B12, wherein the antibody construct comprises a second TAA antigen-binding domain.
B14. The antibody construct according to embodiment B13, wherein the first and second TAA antigen-binding domain bind to the same TAA.
B15. The antibody construct according to any one of embodiments B1 to B14, wherein the first TAA antigen-binding domain is a FRα antigen-binding domain.
B16. The antibody construct according to embodiment B15, wherein the FRα antigen-binding domain comprises: a) a heavy chain variable domain comprising the three heavy chain CDR.s of antibody 8K22, and a light chain variable domain comprising the three light chain CDRs of antibody 8K22; or b) heavy chain variable domain comprising the three heavy chain CDRs of antibody 1H06, and a light chain variable domain comprising the three light chain CDRs of antibody 1H06.
B17. The antibody construct according to embodiment B16, wherein the FRα antigen-binding domain is a human or humanized antigen-binding domain.
B18. The antibody construct according to embodiment B17, wherein the FRα antigen-binding domain comprises:
a) a heavy chain variable domain (VH) sequence that is at least 85% identical to the VH sequence of v23794 and a light chain variable domain (VL) sequence that is at least 85% identical to the VI.: sequence of v23794;
b) a heavy chain variable domain (VH) sequence that is at least 85% identical to the VH sequence of v23795 and a light chain variable domain (VL) sequence that is at least 85% identical to the VL sequence of v23795;
c) a heavy chain variable domain (VH) sequence that is at least 85% identical to the VH sequence of v23796 and a light chain variable domain (VL) sequence that is at least 85% identical to the VL sequence of v23796;
d) a heavy chain variable domain (VH) sequence that is at least 85% identical to the VET sequence of v23797 and a light chain variable domain (VL) sequence that is at least 85% identical to the VL sequence of v23797;
e) a heavy chain variable domain (VH) sequence that is at least 85% identical to the VH sequence of v23798 and a light chain variable domain (VL) sequence that is at least 85% identical to the VL sequence of v23798;
f) a heavy chain variable domain (VH) sequence that is at least 85% identical to the VH sequence of v23799 and a light chain variable domain (VL) sequence that is at least 85% identical to the VL sequence of v23799;
g) a heavy chain variable domain (VH) sequence that is at least 85% identical to the VH sequence of v23800 and a light chain variable domain (VL) sequence that is at least 85% identical to the VL sequence of v23800;
h) a heavy chain variable domain (VH) sequence that is at least 85% identical to the sequence of v23801 and a light chain variable domain (VL) sequence that is at least 85% identical to the VL sequence of v23801;
i) a heavy chain variable domain (VH) sequence that is at least 85% identical to the VH sequence of v23802 and a light chain variable domain (VL) sequence that is at least 85% identical to the VL sequence of v23802;
j) a heavy chain variable domain (VH) sequence that is at least 85% identical to the VH sequence of v23803 and a light chain variable domain (VL) sequence that is at least 85% identical to the VL sequence of v23803;
k) a heavy chain variable domain (VH) sequence that is at least 85% identical to the sequence of v23804 and a light chain variable domain (VL) sequence that is at least 85% identical to the VL sequence of v23804;
l) a heavy chain variable domain (VH) sequence that is at least 85% identical to the VH sequence of v23805 and a light chain variable domain (VL) sequence that is at least 85% identical to the VL sequence of v23805;
m) a heavy chain variable domain (VH) sequence that is at least 85% identical to the VH sequence of v23806 and a light chain variable domain (VL) sequence that is at least 85% identical to the VL sequence of v23806;
n) a heavy chain variable domain (VH) sequence that is at least 85% identical to the VH sequence of v23807 and a light chain variable domain (VL) sequence that is at least 85% identical to the VL sequence of v23807;
o) a heavy chain variable domain (VH) sequence that is at least 85% identical to the VH sequence of v23808 and a light chain variable domain (VL) sequence that is at least 85% identical to the VL sequence of v23808;
p) a heavy chain variable domain (VH) sequence that is at least 85% identical to the VH sequence of v23809 and a light chain variable domain (VL) sequence that is at least 85% identical to the VL sequence of v23809;
q) a heavy chain variable domain (VH) sequence that is at least 85% identical to the VH sequence of v23810 and a light chain variable domain (VL) sequence that is at least 85% identical to the VL sequence of v23810;
r) a heavy chain variable domain (VH) sequence that is at least 85% identical to the VH sequence of v23811 and a light chain variable domain (VL) sequence that is at least 85% identical to the VL sequence of v23811;
s) a heavy chain variable domain (VH) sequence that is at least 85% identical to the VH sequence of v23812 and a light chain variable domain (VL) sequence that is at least 85% identical to the VL sequence of v23812;
t) a heavy chain variable domain (VH) sequence that is at least 85% identical to the VH sequence of v23813 and a light chain variable domain (VL) sequence that is at least 85% identical to the VL sequence of v23813;
u) a heavy chain variable domain (VH) sequence that is at least 85% identical to the VH sequence of v23814 and a light chain variable domain (VL) sequence that is at least 85% identical to the VI: sequence of v23814;
v) a heavy chain variable domain (VH) sequence that is at least 85% identical to the VH sequence of v23815 and a light chain variable domain (VL) sequence that is at least 85% identical to the VL sequence of v23815;
w) a heavy chain variable domain (VH) sequence that is at least 85% identical to the VH sequence of v23816 and a light chain variable domain (VL) sequence that is at least 85% identical to the VL sequence of v23816;
x) a heavy chain variable domain (VH) sequence that is at least 85% identical to the VH. sequence of v23817 and a light chain variable domain (VL) sequence that is at least 85% identical to the VL sequence of v23817; or
y) a heavy chain variable domain (VH) sequence that is at least 85% identical to the VH sequence of v23818 and a light chain variable domain (VL) sequence that is at least 85% identical to the VL sequence of v23818.
B19. The antibody construct according to any one of embodiments B1 to B18, wherein the TAA antigen-binding domain is in an scFv format.
B20. The antibody construct according to any one of embodiments B1 to 1318, wherein the TAA antigen-binding, domain is in a Fab format.

B21. The antibody construct according to any one of embodiments B1 to B20, wherein the scaffold is a dimeric Fc: construct having a first Fc polypeptide and a second. Fc polypeptide, each F′e, polypeptide comprising a CH3 sequence, or wherein the scaffold is a linker or an albumin polypeptide.

B22. The antibody construct according to embodiment B21, wherein the scaffold is a heterodimeric Fc construct having a first Fc polypeptide that is different from the second Fc polypeptide, and wherein the CH3 sequences of the first Fc polypeptide and the second Fc poly peptide comprise amino acid substitutions that promote the formation of a heterodimeric Fc.

B23. The antibody construct according to embodiment B22, wherein:
a) one Fc polypeptide comprises the amino acid substitutions T350V_L351Y_F405A_Y407V and the other Fc polypeptide comprises the amino acid substitutions 17350V_T366L_K392L_T394W;
b) one Fc polypeptide comprises the amino acid substitutions T350V_T366L_K392M_T394W and the other Fc polypeptide comprises the amino acid substitutions T350V_L351Y_F405A_Y407V;
c) one Fc polypeptide comprises the amino acid substitutions L351Y_F405A_Y407V and the other Fc polypeptide comprises the amino acid substitutions T366L_K392M_T394W;
d) one Fc polypeptide comprises the amino acid substitutions L351Y_F405A_Y407V and the other Fc polypeptide comprises the amino acid substitutions T366L_ K392L_T394W; or
e) one Fc polypeptide comprises the amino acid substitutions L351Y_S400E_F405A_Y407V and the other Fc polypeptide comprises the amino acid substitutions T366O_N390R_K392M_T394W,
   wherein the numbering of residues is according to the EU numbering system,
B24. The antibody construct according to any one of embodiments B21 or B23, further comprising one or more amino acid modifications that reduce effector function,
B25. The antibody construct according to embodiment B24, wherein the one or more amino acid modifications are L. 234A, L235A and D265S, wherein the numbering of residues is according to the EU numbering system.
B26. The antibody construct according to any one of embodiments B1 to B25, wherein the first 4-1BB antigen-binding domain is linked to the N terminus of the first Fc polypeptide, and the first TAA antigen-binding domain is linked to the C terminus of the first Fc polypeptide.
B27. The antibody construct according to any one of embodiments B1 to B25, wherein the first 4-1BB antigen-binding, domain is linked to the N terminus of the first Fc polypeptide, and the first TAA antigen-binding domain is linked to the C terminus of the second Fc polypeptide
B28. The antibody construct according to embodiment B26 or B27, further comprising a second 4-1BB antigen-binding domain linked to the N terminus of the second Fc polypeptide.
B29. The antibody construct according to any one of embodiments B1 to B25, comprising a first 4-1BB antigen-binding domain linked to the N terminus of the first Fc polypeptide, a second 4-1BB antigen-binding domain linked to the N terminus of the second Fc polypeptide, a first TAA antigen-binding domain linked to the C terminus of the first Fc polypeptide and a second TAA antigen-binding domain linked to the C terminus of the second Fc polypeptide.
B30. The antibody construct according to any one of embodiments B1 to B25, comprising a first 4-1BB antigen-binding domain linked to the N terminus of the first Fc polypeptide or to the N terminus of the second Fc polypeptide, a first TAA antigen-binding domain linked to the C terminus of the first Fc polypeptide and a second TAA antigen-binding domain linked to the C terminus of the second Fc polypeptide.
B31. The antibody construct according to any one of embodiments B1 to B30, wherein the first and or second 4-1BB antigen-binding domain comprises a heavy chain variable domain comprising the three heavy chain CDRs of antibody 1G1, and a light chain variable domain comprising the three light chain CDRs of antibody 1G1, and the first and/or second Frα antigen-binding domain comprises a heavy chain variable domain comprising the three heavy chain CDRs of antibody 8K22, and a light chain variable domain comprising the three light chain CDRs of antibody 8K22.
B32. The antibody construct according to embodiment B31, wherein the first and second 4-1BB antigen-binding domain comprises a heavy chain variable domain (VH) sequence that is at least 85% identical to the VH sequence of v28614 and a light chain variable domain (VL) sequence that is at least 85% identical to the VL sequence of v28614, and the first and/or second FRα antigen-binding domain comprises a heavy chain variable domain (VH) sequence that is at least 85% identical to the VH sequence of v23807 and a light chain variable domain (VL) sequence that is at least 85% identical to the VL sequence of v23807.
B33. The antibody construct according to embodiment B32, comprising a first heavy chain polypeptide sequence as set forth in SEQ ID NO:353, a second heavy chain polypeptide sequence as set forth in SEQ ID NO:349, and a light chain polypeptide sequence as set forth in SEQ NO:346.
B34. The antibody construct according to any one of embodiments B1 to B33, conjugated to a drug.
B35. A pharmaceutical composition comprising the antibody construct of any one of embodiments B1 to B33.
B36. One or more nucleic acids encoding the antibody construct according to any one of embodiments B1 to B34.
B37, One or more vectors comprising the one or more nucleic acids according to embodiment B36.
B38. An isolated cell comprising the one or more nucleic acid according to embodiment B36, or the one or more vectors according to embodiment B37,
B39. A method of preparing the antibody construct according to any one of embodiments B1 to B34, comprising culturing the isolated cell of embodiment B38 under conditions suitable for expressing the antibody construct, and purifying the antibody construct.
B40. A method of treating a subject having a cancer, comprising administering to the subject an effective amount of the antibody construct according to any one of embodiments B1 to B34.
B41. Use of an effective amount of the antibody construct according to any one of embodiments B1 to B34 for the treatment of cancer in a subject in need thereof.
B42. Use of the antibody construct according to any one of embodiments B1 to B34 in the preparation of a medicament for the treatment of cancer.

B43. The antibody construct according to any one of embodiments B1 to B34, for use in the treatment of cancer in a subject.
C1. An antibody construct or antigen-binding fragment thereof, that specifically binds to 4-1BB, comprising: a heavy chain variable sequence comprising three heavy chain CDRs and a light chain variable sequence comprising three light chain CDRs, wherein the heavy chain CDRs and the light chain CDRs are from any one of antibodies 1G1, 1B2, 1C3, 1C8, 2A7, 2E8, 2H9, 3D7, 3H1, 3E7, 3G4, 4B11, 4E6, 4F9, 4G10, 5E2, 5G8, or 6B3.
C2. The antibody construct according to embodiment C1, wherein the antibody construct agonizes 4-1BB.
C3. The antibody construct according to embodiment C2, comprising a heavy chain variable (VH) sequence comprising three CDRs and a light chain variable (VL) sequence comprising three CDRs, wherein the heavy chain CDRs and the light chain CDRs are from any one of antibodies 1G1, 1C3, 1C8, 2E8, 3E7, 4E6, 5G8, or 6B3.
C4. The antibody construct according to any one of embodiments C1 to C3, wherein the antibody or antigen-binding fragment is or comprises a humanized antibody.
C5. The antibody construct according to embodiment C1 or C2, comprising a VH sequence and a VL sequence having at least 85% sequence identity to the VH and VL sequences of any one of variants v28726, v28727, v28728, v28730, v28700, v28704, v28705, v28706, v28711, v28712, v28713, v28696, v28697, v28698, v28701, v28702, v28703, v28707, v28683, v28684, v28685, v28686, v28687, v28688, v28689, v28690, v28691, v28692, v28694, or v28695.
C6. The antibody construct according to any one of embodiments C1 to C5, wherein the antibody or antigen-binding fragment has a binding affinity (K_{D}) for a human 4-1BB molecule of about 10 nM to about 500 nM.
C7. The antibody construct according to any one of embodiments C1 to C6, wherein the antibody or antigen-binding fragment binds to an epitope within the extracellular domain of human 4-1BB polypeptide.
C8. The antibody construct according to any one of embodiments C1 to C7, wherein the antibody construct includes immunoglobulin constant domains, wherein the constant domains are from an IgG1 or a variant thereof, an IgG2 or a variant thereof, an IgG4 or a variant thereof, an IgA or a variant thereof, an IgE or a variant thereof, an IgM or a variant thereof, or an IgD or a variant thereof.
C9. The antibody construct according to any one of embodiments C1 to C8, wherein the antibody is or comprises a human IgG1.
C10. The antibody construct according to any one of embodiments C1 to C9, wherein the antibody or antigen-binding fragment is a monoclonal antibody.
C11.The antibody construct according to any one of embodiments C1 to C7, wherein the antibody fragment is a Fab fragment, a Fab′ fragment, a F(ab′)2 fragment, a Fv fragment, a scFv fragment, a single domain antibody, or a diabody.
C12. The antibody construct according to any one of embodiments C1 to C11, conjugated to a drug.
C13. A pharmaceutical composition comprising the antibody construct of any one of embodiments C1 to C12.
C14. One or more nucleic acids encoding the antibody construct according to any one of embodiments C1 to C11.
C15. One or more vectors comprising the one or more nucleic acids according to embodiment C14.
C16. An isolated cell comprising the one or more nucleic acids according to embodiment C14, or the one or more vectors according to embodiment C15.
C17. A method of preparing the antibody construct according to any one of embodiments C1 to C12, comprising culturing the isolated cell of embodiment C16 under conditions suitable for expressing the antibody construct, and purifying the antibody construct.
C18. A method of treating a subject having a cancer, comprising administering to the subject an effective amount of the antibody construct according to any one of embodiments C1 to C12.
C19. Use of an effective amount of the antibody construct according to any one of embodiments C1 to C12 for the treatment of cancer in a subject in need thereof.
C20. Use of the antibody construct according to any one of embodiments C1 to C12 in the preparation of a medicament for the treatment of cancer.
C21. The antibody construct according to any one of embodiments C1 to C12, for use in the treatment of cancer in a subject.
D1. An antibody construct or antigen-binding fragment thereof, that specifically binds to FRα, comprising: a heavy chain variable (VH) sequence comprising three CDRs and a light chain variable (VL) sequence comprising three CDRs, wherein the heavy chain CDRs and the light chain CDRs are from antibody 8K22 or 1H06.
D2. The anti-FRα antibody or antigen-binding fragment according to embodiment D1, wherein the antibody or antigen-binding fragment thereof is or comprises a humanized antibody.
D3. The anti-Frα antibody or antigen-binding fragment according to embodiment D1 or D2, comprising a VH sequence and a VL sequence having at least 85% sequence identity to the VH and VL sequences of any one of variants 23794, 23795, 23796, 23797, 23798, 23799, 23800, 23801, 23802, 23803, 23804, 23805, 23806, 23807, 23808, 23809, 23810, 23811, 23812, 23813, 23814, 23815, 23816, 23817, or 23818.
D4. The anti-FRα antibody or antigen-binding fragment according to embodiment D1 or D2, comprising a VH sequence having at least 85% sequence identity to the VH sequence as set forth in SEQ ID NO:300 and a VL sequence having at least 85% sequence identity to the VL sequence as set forth in SEQ ID NO:301.
D5. The anti-FRα antibody or antigen-binding fragment according to any one of embodiments D1 to D4, wherein the antibody or antigen-binding fragment has a binding affinity (K_{D}) fora human FRα molecule of between about 100 pM to about 100 nM.
D6. The anti-FRα antibody or antigen-binding fragment according to any one of embodiments D1 to D5, wherein the antibody includes an immunoglobulin constant domain, wherein the constant domain is selected from an IgG1 or a variant thereof, an IgG2 or a variant thereof, an IgG4 or a variant thereof, an IgA or a variant thereof, an IgE or a variant thereof, an IgM or a variant thereof, and an IgD or a variant thereof.
D7. The anti-FRα antibody or antigen-binding fragment according to any one of embodiments D1 to D6, wherein the antibody is or comprises a human IgG1.

D8. The anti-FRα antibody or antigen-binding fragment according to any one of embodiments D1 to D7, wherein the antibody or antigen-binding fragment is a monoclonal antibody.
D9. The anti-FRα antibody or antigen-binding fragment according to any one of embodiments D1 to D8, wherein the antibody fragment is a Fab fragment, a Fab′ fragment, a F(ab′)2 fragment, a Fv fragment, a scFv fragment, a single domain antibody, or a diabody.
D10. The antibody construct according to any one of embodiments D1 to D9, conjugated to a drug.
D11. A pharmaceutical composition comprising the antibody construct of any one of embodiments D1 to D10.
D12. One or more nucleic acids encoding the antibody construct according to any one of embodiments D1 to D9.
D13, One or more vectors comprising the one or more nucleic acids according to embodiment D12.
D14. An isolated cell comprising the one or more nucleic acids according to embodiment D12, or the one or more vectors according to embodiment D13.
D15. A method of preparing the antibody construct according to any one of embodiments D1 to D10, comprising culturing the isolated cell of embodiment D14 under conditions suitable for expressing the antibody construct, and purifying the antibody construct.
D16. A method of treating a subject having a cancer, comprising administering to the subject an effective amount of the antibody construct according to any one of embodiments D1 to D10.
D17. Use of an effective amount of the antibody construct according to any one of embodiments D1 to D10 for the treatment of cancer in a subject in need thereof.
D18. Use of the antibody construct according to any one of embodiments D1 to D10 in the preparation of a medicament for the treatment of cancer.
D19. The antibody construct according to any one of embodiments D1 to D10, for use in the treatment of cancer in a subject.

### EXAMPLES

Below are examples of specific embodiments related to the antibody constructs described herein. The examples are offered for illustrative purposes only, and are not intended to limit the scope of the disclosure in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers. Inc., current addition); Sambrook, et al., Molecular Cloning: A Laboratory Manual (2nd Edition, 1989); Methods In Enzymology (S. Colowick and N. Kaplan eds., Academic Press, Inc); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Design and Preparation of Exemplary 4-1BB×HER2 Bispecific Antibody Constructs

A number of exemplary bispecific antibody constructs (or bispecific antibodies) targeting 4-1BB and the TAA HER2, as well as controls were constructed as described below. The antibodies and controls were prepared in different exemplary formats, as described in FIG. 2. These antibody constructs were prepared in order to allow examination of the potential for conditional agonist of 4-1BB, and the optimal format for activity of the 4-1BB×HER2 constructs.

### Design of Exemplary Bispecific Antibody Constructs Targeting 4-1BB and HER2

Bispecific antibody constructs were prepared in a format in which the HER2 antigen-binding domain was an scFv and the 4-1BB antigen-binding domain was a Fab. Constructs, including controls, comprised an IgGI Fc, unless otherwise indicated (see Table 1). These bispecific antibody constructs comprised a human IgG1 heterodimeric Fc having sets of CH3 domain amino acid substitutions promoting the formation of a heterodimeric Fc These sets of amino acid substitutions are referred to herein as Het FcA (having the amino acid substitutions T350V/L351Y/F405A/Y407V) and Het FcB having the amino acid substitutions T350VIT366L/K392L/T394W). Variants having these sets of amino acid substitutions are referred to in Table 1 as having “Het Fv” modifications. Variants in Table 1 noted as having “FcKO” have the following CH2 amino acid substitutions which knock out FcγR binding: L234A, L235A and D265S. Amino acid residues in the Fc region are identified according to the EU index.

Bivalent, trivalent and tetravalent antibody constructs were made, all having three polypeptide chains one heavy chain containing Het FcA mutations, a second heavy chain containing Het FcB mutations and a single light chain. The heavy chains were constructed in a series of formats, all of which were comprised of one or two anti-4-1BB antigen-binding domains in the Fab format and a single anti-HER2 antigen-binding domain in the scFv format. These heavy chain formats are described below from N-terminus to C-terminus:

VL-VH-VH-CH1hinge-CH2-CH3

VH-CH 1-hinge-CH2-CH3-VL-VH

VH-CH1-hinge-CH2-CH3

VL-VH-hinge-CH2-CH3

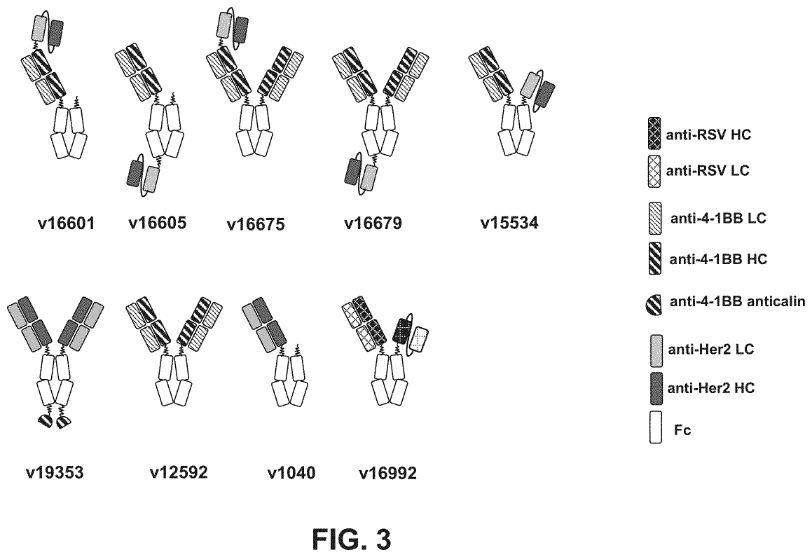

Table 1 provides a description of the 4-1BB×HER2 bispecific antibody constructs that were prepared. The number of 4-1BB-targeting domains and HER2-targeting domains are indicated in the “Format” column. For example, in Table 1, 1×1 indicates that the bispecific antibody construct has one 4-1BB binding domain and one HER2 binding domain, 2×1 indicates that the bispecific antibody construct has two 4-1BB binding domains and one HER2 binding domain, etc. The formats of the specific bispecific antibody constructs described below are also represented in FIG. 3.

TABLE 1
Exemplary bispecific antibody constructs targeting 4-1BB and HER2

| | | Format | Fc | |
|---|---|---|---|---|
| Variant | Paratopes | (4-1BB × HER2) | description | Description |
| v16601 | N-terminal trastuzumab scFv, | 1 × 1 | Het Fc, FcKO | 4-1BB × HER2 |
| | monovalent MOR7480.1 4-1BB | | | bispecific antibody |
| | | | | construct |
| v16605 | C-terminal trastuzumab scFv, | 1 × 1 | Het Fc, FcKO | 4-1BB × HER2 |
| | monovalent MOR7480.1 4-1BB | | | bispecific antibody |
| | | | | construct |
| v16675 | N-terminal trastuzumab scFv, | 2 × 1 | Het Fc, FcKO | 4-1BB × HER2 |
| | bivalent MOR7480.1 4-1BB | | | bispecific antibody |
| | | | | construct |
| v16679 | C-terminal trastuzumab scFv, | 2 × 1 | Het Fc, FcKO | 4-1BB × HER2 |
| | bivalent MOR7480.1 4-1BB | | | bispecific antibody |
| | | | | construct |
| v15534 | Trastuzumab scFv, | 1 × 1 | Het Fc | 4-1BB × HER2 |
| | monovalent MOR7480.1 4-1BB | | | bispecific antibody |
| | | | | construct |
| v19353 | Trastuzumab, C-terminal 4-1BB, | 2 × 2 | IgG4 with L234A, | control 4-1BB × |
| | 4-1BB binding domains | | L235A mutations | HER2 bispecific |
| | are anticalins | | | construct (described |
| | | | | in WO2016/177802) |
| v12592 | MOR7840.1, no HER2- | 2 × 0 | Het Fc | control construct, |
| | binding arm | | | monospecific anti- |
| | | | | 4-1BB antibody |
| v13725 | CR8071, negative control | 0 × 0 | HetFc | fully human anti- |
| | antibody | | | influenza HA |
| | | | | antibody |
| v16992 | Pavilizumab, negative control | 0 × 0 | HetFc, FcKO | human anti-RSV |
| | antibody | | | antibody |
| v1040 | trastuzumab, one-armed | 0 × 1 | Het Fc | control HER2- |
| | antibody (Fab format) | | | binding antibody |

The VH and VL sequences used to construct the 4-1BB antigen-binding domains of the constructs are provided in Table 15, as are the sclFv sequences used to construct HER2 scFv containing constructs. Table X identifies the clones that make up each of the antibody constructs. The polypeptide sequences of each done can be found in Table Y.

### Production of 4-1BB×HER2 Bispecific Antibodies

To allow the production of bi specific antibodies, heavy chain vectors having a 5′-EcoR1 restriction site signal peptide heavy chain clone terminating at G446 (EU numbering) of CH3-TGA stop-BamH1 cutsite-3′, were ligated into a pTT5 vector to produce heavy chain expression vectors. Light chain vectors, having a 5′-EcoRI cut site-signal peptide-light chain-TGA stop-cutsite-3′, were ligated into a pTT5 vector (Durocher Y et al., Nucl. Acids Res. 2002; 30, No.2 e9) to produce light chain expression vectors. The resulting heavy and light chain expression vectors were sequenced to confirm correct reading frame and sequence of the coding DNA. One of two signal peptides was used, either an artificially designed sequence, MRPTWAWWLFLVLLLALWAPARG [SEQ IU NO:1], Barash S et al., Biochem and Biophys Res. Comm, 2002; 294, 835-842) or the HLA-A signal peptide MAVMAPRTLVLLLSGALALTQTWAG [SEQ ID NO:2].

The heavy and light chains of variants, were expressed in 200 ml cultures of CHO-3E7 cells. CHO-3E7 cells, at a density of 1.7-2×10⁶ cells/ml, were cultured at 37° C. in FreeStyle™ F17 medium (Thermo Fisher, Watham, Mass.) supplemented with 4 mM glutamine (GE Life Sciences, Marlborough, Mass.) and 0.1% KoliphorP188 (Sigma Aldrich, St. Louis, Mo.). A total volume of 200 ml was transfected with a total of 200 μg DNA (100 μg of variant DNA and 100 μg of CFP/AKT/stuffer DNA) using PEI-max (Polyscience, Philadelphia, Pa.) at a DNA:PEI ratio of 1:4 (W/W), Twenty-four hours after the addition of the DNA-PEI mixture, 0.5 mM valproic acid (final concentration)+1% w/v Tryptone (final concentration)+1× antibiotic/antimycotics (GE Life Sciences, Marlborough, Mass.) were added to the cells which were then transferred to 32° C. and incubated for 7 days prior to harvesting.

Clarified supernatant samples were incubated in batch with MabSelect™ SuRe™ resin (GE Healthcare, Chicago, Ill.) cleaned with NaOH and equilibrated in DPBS. Resin was poured into cleaned columns, the columns were washed with DPBS and protein eluted with 100 mM sodium citrate buffer pH 3.0. The eluted antibodies were pH adjusted by adding 10% (v/v) 1M HEPES pH 8 to yield a final pH of 6-7. Samples were buffer exchanged into PBS and aseptically filtered. Protein was quantified based on A280 nm (NanoDrop™). Endotoxin levels were determined using the Endosafe® Portable system (Charles River, Wilmington, Mass.). For samples above 0.1 EU/mg, these underwent endotoxin removal with the Proteus NoEndo™ Spin columns (Charles River, Wilmington, Mass.).

Post protein-A purification, samples were either buffer exchanged into DPBS and aseptically filtered or, depending on their homogeneity assessed by UPLC-SEC, subjected to SEC purification. Samples were loaded onto a Superdex 200 10/30 Increase column (GE Healthcare Life Sciences, Marlborough, Mass.) on an Akta Avant 25 chromatography system (GE Healthcare Life Sciences, Marlborough, Mass.) in DBPS with a flow rate of 0.5 mL/min. Fractions of eluted protein were collected based on A280 nm and the fractions were assessed by non-reducing and reducing High Throughput Protein Express assay using Caliper LabChip GXII (Perkin Elmer, Waltham, Mass.). Procedures were carried out according to HT Protein Express LabChip User Guide version2 LabChip GXII User Manual, with the following modifications. antibody samples, at either 2 μl or 5 μl (concentration range 5-2000 ng/μl), were added to separate wells in 96 well plates (BioRad, Hercules, Calif.) along with 7 μl of HT Protein Express Sample Buffer (Perkin Elmer #760328). Antibody samples were then denatured at 70° C. for 15 mins. The LabChip instrument was operated using the HT Protein Express Chip (Perkin Elmer, Waltham, Mass.) and the Ab-200 assay setting.

Endotoxin levels were determined by the LAL (limulus amebocyte lysate) assay using the Endosafe® Portable Test System (PTS, Charles River, Wilmington, Mass.). Protein was quantified based on A280 nm (Nanodrop) post protein-A and SEC.

UPLC-SEC was performed using a Waters Acquity BEH200 SEC column (2.5 mL, 4.6×150 mm, stainless steel, 1.7 μm particles) (Waters LTD, Mississauga, ON) set to 30° C. and mounted on a Waters Acquity UPLC H-Class Bio system with a PDA detector. Run times consisted of 7 min and a total volume per injection of 2.8 mL with a running buffer of DPBS or DPBS with 0.02% Tween 20 pH 7.4 at 0.4 ml/min. Elution was monitored by UV absorbance in the range 210-50 nm, and chromatograms were extracted at 280 nm. Peak integration was performed using Empower 3 software.

### Purity Assessment of Bispecific Antibodies by LC/MS

The apparent purity of the variants was assessed using mass spectrometry after purification and non-denaturating deglycosylation as described below.

As the antibody contained Fc N-linked glycans only, the samples were treated with only one enzyme, N-glycosidase F (PNGase-F). The purified samples were de-glycosylated with PINGaseF as follows: 0.1U PINGaseF/μg of antibody in 50 mM Tris-HCl pH 7.0, overnight incubation at 37° C., final protein concentration of 0.48 mg/mL. After deglycosylation, the samples were stored at 4° C. prior to LC-MS analysis.

The deglycosylated protein samples were analyzed by intact LC-MS using an Agilent 1100 HPLC system coupled to an LTQ-Orbitrap XL mass spectrometer (ThermoFisher , Waltham, Mass.) via an Ion Max electrospray source. The samples (5 μg) were injected onto a 2.1×30 mm Poros R2 reverse phase column (Applied Biosystems) and resolved using the following gradient conditions: 0-3 min: 20% solvent B; 3-6 min: 20-90% solvent B; 6-7 min: 90-20% Solvent B; 7-9 min: 20% solvent B. Solvent A was degassed 0.1% formic acid aq. and solvent B was degassed acetonitrile. The flow rate was 3 mL/mire. The flow was split post-column to direct 100 μL/mL into the electrospray interface. The column was heated to 82.5° C. and solvents were heated pre-column to 80° C. to improve protein peak shape. Prior to analysis, the LTQ-Orbitrap XL was calibrated using ThermoFisher Scientific's LTQ Positive Ion ESI calibration solution (caffeine, MRFA and Ultramark 1621), and tuned for optimal detection of larger proteins (>50 kDa) using a 1 mg/mL solution of lactalbumin. The cone voltage (source fragmentation setting) was approximately 40 V. the FT resolution was 7,500 and the scan range was m/z 400-4,000. The LC-MS system was evaluated for IgG sample analysis using a deglycosylated IgG standard (Waters IgG standard) as well as a deglycosyated antibody standard mix (25:75 half full sized antibody).

For each LC-MS analysis the mass spectra acquired across the antibody peak (typically 3.6-4.1 minutes) were summed and the entire multiply charged ion envelope (m/z 1,400-4,000) was deconvoluted into a molecular weight profile using the MaxEnt 1 module of MassLynx™, the instrument control and data analysis software (Waters LTD, Missassaugua, ON). Briefly, the raw protein LC-MS data were first opened in QualBrowser, the spectrum viewing module of Xcalibur™ (Thermo Fisher, Waltham, Mass.) and converted to be compatible with Ma.ssLynx™ using Databridge™, a file conversion program provided by Waters. The converted protein spectra were viewed in the Spectrum module of MassLynx™ and deconvoluted using MaxEnt 1. The apparent amount of each antibody species in each sample was determined from their peak heights in the resulting molecular weight profiles. In the majority of cases, the antibodies comprised >95% of the desired construct, with no major glycovariants.

### Example2

### Ability of 4-1BB x HER2 Bispecific Antibody Constructs to Bind to 4-1BB and HER2 as Assessed by Surface Plasmon Resonance (SPR)

To check the production and characteristics of the 4-1BB×HER2 bispecific antibodies described in Example 1, the ability of these antibodies to bind to human 4-1BB and HER2 was assessed by SPR. 4-1BB×HER2 bispecific variants 16601, 16605, 16675, 16679 were assessed, as well as the control antibody variant 19353 (trastuzumab, having two anticalin domains at the C-termini of both heavy chains that bind to 4-1BB).

### Binding of 4-1BB to Antibodies by SPR

A surface plasmon resonance (SPR) binding assay for determination of 4-1BB binding affinity to 4-1BB antibody variants was carried out on Biacore™ T200 instrument (GE Healthcare, Mississauga, ON, Canada) with PBS-T (PBS+0.05% (v/v) Tween 20) running buffer (with 0.5 M EDTA stock solution added to 3.0 mM final concentration) at a temperature of 25° C. CMS Series S sensor chip, Biacore™ amine coupling kit (NHS, EDC and 1 M ethanolamine), and 10 mM sodium acetate buffers were all purchased from GE Healthcare. PBS running buffer with 0.05% Tween20 (PBS-71) was purchased from Teknova Inc. (Hollister, Calif.). Goat polyclonal anti-human Fc antibody was purchased from Jackson lmmunoResearch Laboratories Inc. (West Grove, Pa.).

The SPR binding of antibodies to 4-1BB antigen occurred in two steps: an indirect capture of antibodies onto the anti-human Fc-specific polyclonal antibody surface, followed by the injection of five concentrations of purified human monomeric 4-1BB (SEQ ID NO:70). Monomeric 4-1BB protein was produced by cleaving a 4-1BB-Fc fusion protein (v16730). 4-1BB-Fc was expressed and purified with Protein A in the same manner to antibodies in Example 1 above. The construct was made with a factor Xa cleavage site between the 4-1 BB and the Fc, and with a 10× His tag at the c-term of the Fc.

v16730 in dPBS was buffer exchanged into Factor Xa cleavage buffer (20 mM Tris, 100 mM NaCl, 2 mM CaCl2 pH 8) using a 5 mL Zeba spin column (ThermoFisher) and cleaved with 0.45% (w/w) of Factor Xa (New England Biolabs, Whitby, ON, Canada) overnight at room temp. The cleavage reaction was stopped by adding 0.372 μM final concentration of 1,5-Dansyl-Glu-Gly-Arg-chloromethyl ketone (Calbiochem, San Diego, Calif., USA) as an inhibitor. Satisfactory cleavage was verified by NR+R SDS−PAGE. The cleavage reaction was mixture was subsequently applied to a 1 mL HiTrap Ni Sepharose Excel (GE Heathcare) column equilibrated in dPBS and the column washed with 5×CV dPBS. The cleaved 41BB protein was collected in the flow-thru fractions. Residual Fc was removed using Protein A purification by applying the protein sample to mAb Select SuRe by gravity. The flow-through was applied to a Superdex 200 10/30 column equilibrated in dPBS. Fractions corresponding to the major 41BB product were collected and used for SPR.

The anti-human Fc surface was prepared on a CMS Series S sensorchip by standard amine coupling methods as described by the manufacturer (GE Healthcare). Briefly, immediately after EDC/NHS activation, a 25 μg/mL solution of anti-human Fc in 10 mM NaOAc pH 4.5 was injected at a flow rate of 10 μL/min for 420 s until approximately 2000 resonance units (RUs) were immobilized on all four flow cells. The remaining active groups were quenched by a 420 s injection of 1M ethanolamine at 10 μL/min.

Antibodies for analysis were captured onto the anti-Fc surface by injecting 5 μg/mL solutions at a flow rate of 10 μL/min for 60 s. Using single-cycle kinetics, five concentrations of a two-fold dilution series of 4-1BB starting at 40 nM (for both supernatant and purified antibody runs) with a blank buffer control were sequentially injected at 40 μL/min for 180 s with a 600 s dissociation phase, resulting in a set of sensorgrams with a buffer blank reference. Experiments were performed at a constant temperature of 25° C. The anti-human Fc surfaces were regenerated to prepare for the next injection cycle by one pulse of 10 mM Glycine/HCl pH 1.5 for 120 s at 40 μL/min. Blank-subtracted sensorgrams were analyzed using Biacore™ T200 Evaluation Software v3.0. The blank-subtracted sensorgrams were then fit to the 1:1 Langmuir binding model.

### Binding of HER2 to Antibodies by SPR

An anti-Fc capture chip was prepared in the same way as above, then antibodies for analysis at 50 μg/ml were injected into the chip at a flow rate of 10 μl/min for 60 s. Using single-cycle kinetics, five concentrations of a two-fold dilution series of HER2 (having recombinant human Her-2 amino acids 23-652; eBioscience) starting at 40nM (for both supernatant and purified antibody runs) with a blank buffer control were sequentially injected at 50 μL/min for 180 s with a 1800 s dissociation phase, resulting in a set of sensorgrams with a buffer blank reference. Experiments were performed at a constant temperature of 25° C. The anti-human Fc surfaces were regenerated to prepare for the next injection cycle by one pulse of 10 mM Glycine/HCl pH 1.5 for 120 s at 40 μL/min. Blank-subtracted sensorgrams were analyzed using Biacore™ T200 Evaluation Software v3.0. The blank-subtracted sensorgrams were then fit to the 1:1 Langmuir binding model to obtain the ka, kd and KD values shown in Table 2 below.

TABLE 2
Ability of bispecific antibody constructs to bind to target

| | HER2 | | | 4-1BB | | |
|---|---|---|---|---|---|---|
| Variant | ka (1/Ms) | kd (1/s) | KD (M) | ka (1/Ms) | kd (1/s) | KD (M) |
| 16601 | 9.44 × 10⁴ | 7.63 × 10⁻⁵ | 8.09 × 10⁻¹⁰ | 5.61 × 10⁵ | 1.15 × 10⁻² | 2.05 × 10⁻⁸ |
| 16605 | 4.18 × 10⁴ | 7.83 × 10⁻⁵ | 1.88 × 10⁻⁹ | 1.03 × 10⁶ | 1.32 × 10⁻² | 1.29 × 10⁻⁸ |
| 16675 | 1.02 × 10⁵ | 8.08 × 10⁻⁵ | 7.91 × 10⁻¹⁰ | 7.89 × 10⁵ | 1.35 × 10⁻² | 1.71 × 10⁻⁸ |
| 16679 | 4.96 × 10⁴ | 6.61 × 10⁻⁵ | 1.33 × 10⁻⁹ | 1.04 × 10⁶ | 1.34 × 10⁻² | 1.30 × 10⁻⁸ |
| 19353 | 1.1 × 10⁵ | 6.76 × 10⁻⁵ | 6.16 × 10⁻¹⁰ | 1.71 × 10⁴ | 6.01 × 10⁻³ | 3.51 × 10⁻⁷ |

Table 2 shows that the antibodies in different formats are still able to bind to their targets with similar affinity regardless of format. Variants with C-terminal Trastuzumab scFv, v16605 and v16679, showed about a 2-3 fold drop in KD compared to antibodies with N-terminal scFv, but this was judged to be a minor change and not expected to affect function. All 4-1BB antibodies showed similar KD values, with the anti-4-1BB anticalin v19353 showing a lower affinity for 4-1BB compared to the antibodies.

### Example 3

### Ability of 4-1BB×HER2 Bispecific Antibody Constructs to Stimulate 4-1BB Activity in an NF-kB-Luciferase Reporter Assay

To test the ability of bispecific 4-1BB×:HER2 antibodies to stimulate 4-1BB activity in the presence of a tumor cell expressing HER2, a reporter-gene assay was used as a measure of signaling downstream of 4-1BB to a NF-kB reporter driving luciferase. The 4-1BB×HER2 bispecific antibodies tested included variants 16675, 16679, 15534, 16601, and 16605. Control constructs 19353, 1040, 16992, and 12952 were also tested.

The ability of bispecific antibodies to activate 4-1BB in the context of HER+ tumour cells was measured using a co-culture assay. This assay used Jurkat cells engineered to express 4-1BB and a luciferase reporter gene driven by an NF-kB site. This assay measures signaling from 4-1BB on the surface of the cell down to the nucleus. Two different tumour lines were used; SKOV3, which express a high level of HER2, and MDA-MB-468 which express a low level of HER2. If the activation of 4-1BB is HER2 dependent, activation should be seen in co-culture with SKOV3 cells and not MDA-MB-468 cells.

The day prior to the assay white, TC-treated, polystyrene, 384-well plates (Coming) were treated with 40 μL/well of OKT3, mouse-anti-human-CD3 antibody (Biolegend) at 5 μg/mL in phosphate buffered saline (PBS) (Gibco). The plate was sealed to the plate lid by wrapping in parafilm. The plate was incubated overnight at 4° C. The next day, the contents of the plate were aspirated, and the plate was washed with 3 changes of distilled water (120 μL/well) using a 405HT ELISA plate washer (Biotek). The plate was then ready for use in the assay.

Bispecific antibodies were diluted in Assay Buffer (RPMI (Gibco)/1% FBS (Gibco)) to 400 nM (final assay concentration 100 nM). A volume of 15 μL was pipetted into the well of a 384-well plate treated with OKT3 as above receiving the top concentration of the variant. A volume of 5 μL was pipetted into a volume of 10 μL Assay Buffer in the next well for the second highest concentration well and mixed to give a 3-fold dilution. This was repeated for the transfer from the second highest well to the third highest until the lowest concentration well, where the residual 5 μL volume was removed. 10 μL of either SKOV3 or MDA-MB-468 tumour cells, at a density of 2×10⁶ cells/mL, was then added to give 2×10⁴ cells/well. NFκB luc2P/41 BB Thaw-and-Use Jurkat cells (Promega) were thawed at 37C according to manufacturer's instructions and diluted with 5.8 ML of Assay Buffer. A volume of 20 μL of the reporter cell suspension at approximately 1×10⁶ cells/mL (˜2×10⁴ cells) was added to each well containing the variant/effector-cell mixture.

The co-cultured cells with variants were then incubated at 37° C. in a 5% CO₂ atmosphere for 5 hours and then equilibrated to room temperature on the benchtop for 10 minutes. A volume of 40 μL of Bio-Glo™ (Promega) luciferase substrate reagent was added to each well of the plate and incubated for 10 minutes at room temperature. The plate was scanned on the Synergy™ H1(Biotek) multi-mode plate reader in luminescence mode. Data was analysed using Prism 7 (GraphPad) and four-parameter variable slope nonlinear fit.

### Results

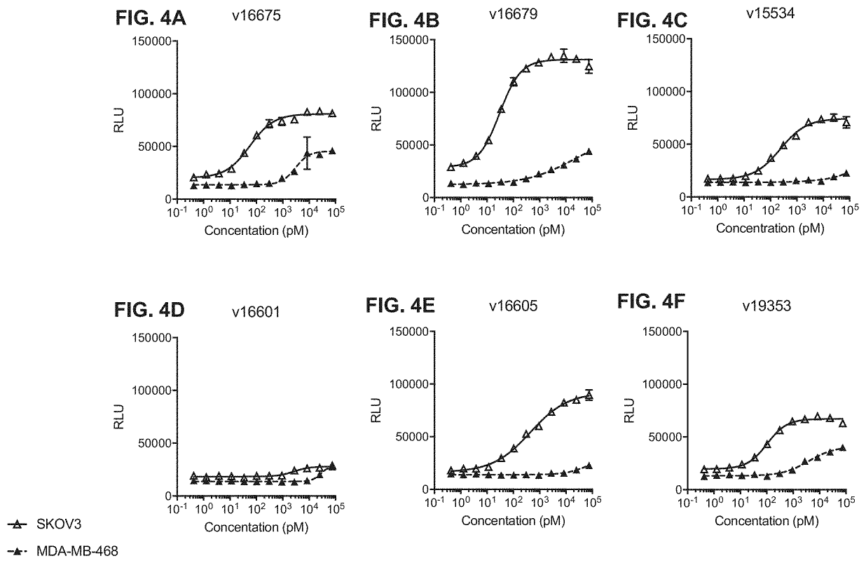

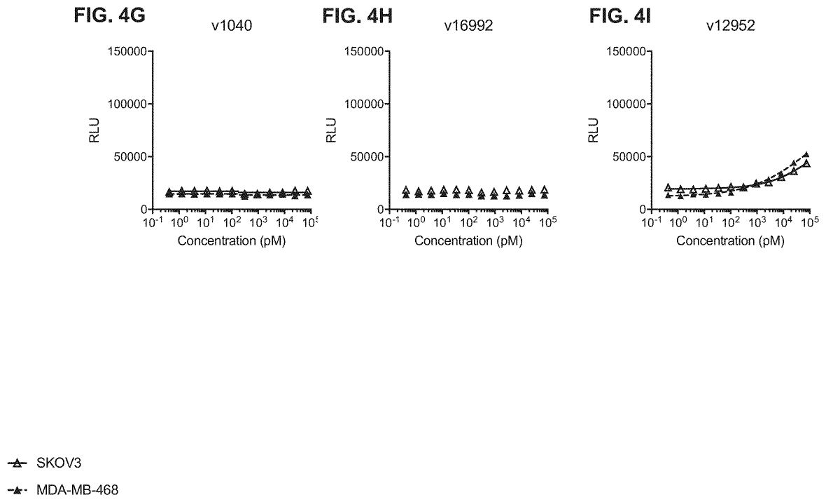

All variants having a 4-1BB binding arm induced dose-dependent NF-kB signalling downstream of 4-1BB as measured by production of luciferase in this assay when co-cultured with SKOV-3 cells (FIG. 4A to 4I). In comparison, variants having activity showed lower activity on MDA-MD-468 cells, suggesting that the presence of HER2 on the surface of the SKOV-3 cells induced cross-linking of the antibodies and enhanced 4-1BB signalling. The lowest activation of 4-1BB was seen with the v16601 variant. v16605 and v16675 showed higher activity. v16679 showed highest activity, as determined by greatest potency (EC50) and activity (max RLU). The positive control 4-1BB×HER2 bispecific antibody v19353 showed intermediate potency—higher than v16601 and v16605 but lower than v16675 and v16679. However, v19353 (having a lipocalin 4-1BB binding domain) showed low activity, as given by maximal RLU, compared to the antibody-based 4-1BB agonists. 4-1BB monospecific control variant v12592 showed low activity at lower concentrations, with activity increasing with concentration; however, activity of this antibody was not increased in the presence of SKOV3 cells compared to MDA-MD-468 cells. Control variant v1040 showed no activity in activating 4-1BB, suggesting that there was no direct effect of the HER2 binding arm on the experiment. v16992 similarly showed no effect of a non-binding control antibody. A summary of the results in provided in Table 3.

TABLE 3
Activity of 4-1BB × HER2 bispecific antibody constructs

| Variant | EC₅₀ (pM) | Max activity (RLU) |
|---|---|---|
| v16601 | 2624 | 27917 |
| v16605 | 400.7 | 92032 |
| v16675 | 56.92 | 80946 |
| v16679 | 30.09 | 131190 |
| v19353 | 101.9 | 67157 |
| v15534 | 237.3 | 74426 |

From this data, it appeared that constructs with two anti-4-1BB binding arms showed greater activity than constructs with one anti-4-1 BB arm. The constructs with a Her2-binding site close to the 4-1BB binding site (eg. v16601 and v16675) appeared less active than the constructs with the Her2 binding site distal from the 4-1BB binding site (eg. v16605 and v16679).

### Example 4

### Primary T Cell-Tumour Co-Culture Assay

The activity of the 4-1BB×HER2 constructs was also compared using primary T cells in co-culture with tumour cells. To look at activation of cells, and the effects of 4-1BB more broadly, production of cytokines such as IFNγ or IL-2 by T cells was used as a proxy for enhanced T cell activation and function. IL-2. is also a key cytokine produced by T cells after activation which promotes their survival and correlates with activation of T cells. This experiment examined the ability of 4-1BB×HER2 antibodies to enhance the activation of T cells as measured by 1L-2 production, where the T cells have been activated by a sub-optimal amount of anti-CD3 antibody. Bispecific 4-1BB×HER2 antibody variants 16601, 16605, 16675, and 16679, were tested in this example, along with the control variants 1040, 12592, and a human IgG1 negative control. The assay was carried out as described below, using CD4+ T cells.

Ahead of the experiment, 96 well plates were coated with anti-CD3 by adding 100 μl 1 μg/ml UCHT1 to wells. The plate was then incubated overnight at 4° C. Blood was obtained from healthy donors, centrifuged at 1500 rpm for 5 minutes and plasma discarded. The blood was then diluted in PBS, layered over Ficoll™ and centrifuged at 2000 rpm for 20 minutes at room temperature. The interface layer of PBMC was then taken, washed with PBS to remove platelets, and resuspended. Cells were then counted, diluted to 5×10⁷ cells per ml in PBS 2% FBS 1mM EDTA, and CD4+ T cell enrichment cocktail (Stemcell Technologies) added at 50 μl/ml cells. The cells were then left at room temperature for 10 minutes. EasySeptm™ D magnetic particles (Stemcell Technologies) were then added at 100 μl/ml cells, mixed and left at room temperature for 5 minutes. The cells were then diluted to a volume of 10 ml using PBS/2% FCS/1 mM EDTA and placed into an EasySep™ magnet. Non-selected cells were then decanted, and placed into a fresh tube in an EasySep™ magnet, and those cells decanted into a fresh tube.

CD4+ T cells were then washed twice in RPMI-1640 10% FCS 1% Penicillin-Streptomycin and diluted to 10⁶ cells/ml and 100 μl added per well to a 96 well plate that had been pre-coated with anti-CD3 (UCHT1). SKBR3 cells were obtained, diluted to 2×10⁵ cells/ml and 50 μl added to wells. Antibody samples were also diluted to 40 nM in RPMI-1640 10% FCS 1% Penicillin-Streptomycin and 50 μl of the resulting solution added per well (10 nM final concentration). In some cases, antibodies were cross-linked using an anti-Fc antibody. The plate was then incubated for three days at 37° C. in a 5% CO₂ atmosphere, and supernatants taken for analysis of IL-2 concentrations by ELISA.

### Results

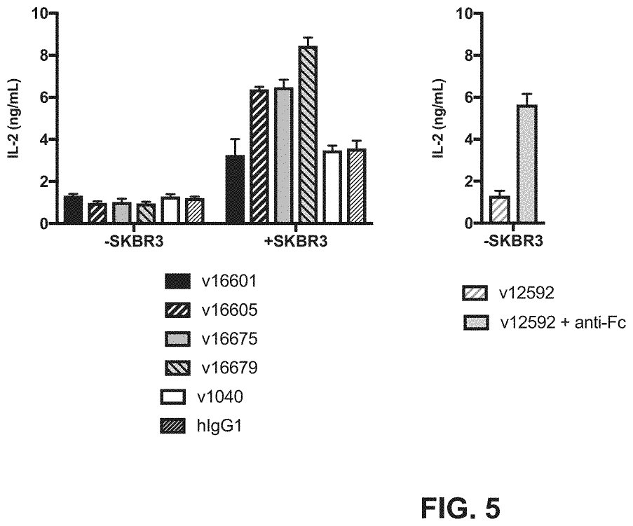

Similar to the 4-1BB NF-kB reporter gene assay, the greatest IL-2 production was seen with v16679, with v16675 and v16605 showing equal levels of IL-2 (FIG. 5, left panel). Without SKBR3 cells in the culture, no increase in IL-2 production was seen as a result of any of the 4-1BB bispecifics. v12592 cross-linked by anti-Fc was used as a positive control and represents the level of signaling induced by a fully cross-linked antibody (FIG. 5, right panel). This data indicated that of the variants tested, v16679 (having the format described in FIG. 2B) was able to induce a level of 4-1BB signaling in the T cells in excess of that stimulated by v12592.

### Example 5

### Comparison of Activation of T Cells by v 16679, v19353 and v12592 as Measured by IfN-γ Production

As v16679 appeared to be the most active 4-1BB×HER2 bispecific in both the reporter-gene assay as well as the primary T cell-tumour co-culture, the ability of this variant to stimulate cytokine production by T cells in co-culture with SKBR3 cells was compared to the positive control constructs v19353 and v12592. In this experiment, IFNγ production was used as a measure of T cell activation as described below.

4-1BB×HER2 bispecific antibodies were prepared at 150 nM in assay media (RPMI containing 5% human AB serum with 1% penicillin-streptomycin (Gibco)). 20 μl of diluted antibodies at 150 nM was then added to the top concentration well of a sterile 384-well cell culture plate (Thermo Scientific), and then the antibodies serially diluted 1:3 to generate the lower antibody concentrations.

SKBR3 tumor cells were cultured in RPMI 10% FCS, treated with 0.05% Trypsin-EDTA (Invitrogen) to remove them from the plate, collected and counted. After centrifugation, the tumor cells were resuspended in assay media at a concentration of 10⁶ cells per ml. 10⁴ tumor cells (10 μl) were added per well according to each condition. Artificial APCs (aAPC/CHO-K1 cells, Promega) were collected using cell dissociation buffer and counted. These cells expressed anti-CD3 (OKT3) and PD-L1 on the surface of the cell and were used to stimulate the T cells in a non-specific manner. After centrifugation, the artificial APC cells were resuspended in assay media at a concentration of 10⁶ cells per ml. T cells were thawed, pelleted, counted and resuspended in assay media at a concentration of 2×10⁶ cells per ml. CD8+ T cells, CD4+ T cells and pan-T cells were purchased from BioIVT, Westbury, N.Y., USA or Stemcell, Vancouver, BC, Canada.

aAPC/CHO-K1 and CD8+, CD4+ T cells or pan-T cells, each from separate donors, were then mixed in 1:2 ratio and 30 μl of cell mixture added to the 384 well plate along with the 10 μl SKBR3 tumour cells. The plate was then incubated for 37° C. in a 5% CO₂ atmosphere.

After four days, supernatant was collected to perform Homogenous Time Resolved Fluorescence ELISA (HTRF™). Either 5 μl supernatants, 5 μl serially diluted IFNγ standards or 5 μl PBS were added to wells of a white round-bottom 384 shallow-well plate (Thermo Scientific). Anti-IFNγ-Cryptate antibody (Cisbio, Bedford, Mass.) and anti-IFNγ-XL (Cisbio, Bedford, Mass.) antibody were diluted 20-fold in detection buffer #3 (Cisbio, Bedford, Mass.), and 20 of each diluted antibody mixed with 11 μl PBS per well. 15 μl of this antibody cocktail was added to wells of the 384 well plate alongside the 5 μl experimental supernatant or standards. The plate was then sealed and left overnight at room temperature. The next day, the plate was read at 665 and 620 nm on a Biotek reader and values reported as ratio of 665 nm/62 0nm readings, after correcting for the plate absorption using PBS-only wells. IFNγ concentrations were calculated using the standard curve. GraphPad Prism v7 was used for data analysis, using the non-linear four-parameter model.

### Results

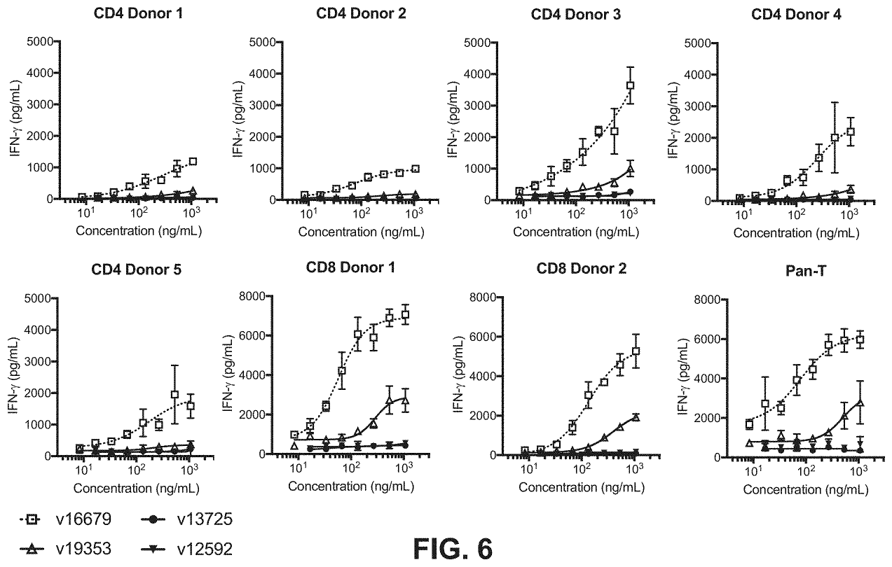

The results are shown in FIG. 6 with multiple independent CD4 and CD8 T cell donors as well as a single pan-T cell donor. This was done to test if the response was only found in a small number of donors. Across all CD4, CD8 and pan-T cell donors, v16679 showed a dose-dependent increase in IFNγ production by cells in co-culture with SKBR3 cells. v16679 also showed much greater maximal cytokine production and higher potency compared to v19353. v12592 was not active in this experiment, and did not show activity greater than that seen by the negative control v13725, suggesting that v16679 is active in conditions where v12592 is not.

### Example 6

### Generation of Antibodies which Bind 4-1BB and Preparation of Mouse-Human Chimeric Antibodies

Additional antibodies targeting 4-1BB were generated by ImmunoPrecise (Victoria, Canada) using their proprietary RapidPrime immunization strategy to immunize mice.

Briefly, Balb/c and NZB/W mice were immunized with human 4-1BB-His protein or a mixture of human 4-1BB-His and mouse 4-1BB-Fc (Acro Biosystems, Newark, Del.), and spleens taken and dissociated to obtain single cells. Splenocytes were then fused with a myeloma paltrier line to create hybridomas. The hybridoma cells were cloned by limiting dilution and the supernatants taken for screening.

Antibodies binding to human, cynomolgus (Macaca fascicularis) and/or mouse (Mus musculus) 4-1BB were identified by ELISA. 96-well plates were coated by adding 100 λL of a 0.1 μg/ml solution of human, cynomolgus or mouse 4-1BB in carbonate buffer (pH 9.6) overnight at 4° C. The wells were then blocked by using 3% skim milk powder in PBS for 1 hour at room temperature, followed by addition of neat hybridoma supernatant (100 μl/well) at 37° C. for 1 hour with shaking. The antibody was then detected using 1:10000 goat anti-mouse IgG/Ig,M (H+L)-HRP, 100 μl/well in PBS 0.05% Tween-20 for 1 hour at 37° C. with shaking. The presence of HRP in the well was then detected using TMB substrate (50 μl/well) for 3 minutes in the dark, followed by addition of 50 μl 1M HCl to stop the reaction. The plate was then read at 450 nm. Antibodies were also counter-screened to exclude antibodies that bound to TNF superfamily members Ox40 and CD40 and GITR, using the same method.

### Results

Twenty-four antibodies binding to human 4-1BB were taken forward to be sequenced and further characterized. Some of these antibodies also bound to cynomolgus or mouse 4-1BB.

### Antibody Recovery

The twenty-four antibodies selected by ELISA were then sequenced to obtain full VH and VL sequences. To prepare RNA from hybridoma cells, cells were washed once in cold phosphate-buffered saline (pH 7.4) and immediately processed through the RNeasy Plus Micro Kit (QIAgen). Total RNA was eluted in nuclease-free water and mRNA converted to cDNA using AMV reverse transcriptase (NEB), primed with oligo (dT₂₀).

Initial PCR of heavy and light chain antibody-coding sequences was performed using primers and methods modified from Babcook et al. (Proc. Natl Acad Sci USA 1996 Jul. 23; 93(15): 7843) and von Boehmer et al. (Nat Protoc. 2016 October; 11(10): 1908), with cDNA as the nucleic acid template. PCR products were cloned into the pCRTOPO4 vector using the Zero Blunt™ TOPO™ PCR Cloning kit (Thermofisher Scientific) and transformed into E. cloni™ cells (Lucigen). Antibiotic-resistant clones were sequenced and analyzed for unique antibody-coding sequences.

A nested PCR reaction was then performed on these unique sequences using V-segment family and J-segment family-specific primers. The resulting amplicons were then cloned into pTT5-based expression plasmids (National Research Council, Montreal, QC). Unique heavy chain sequences and light chain sequences emerging from a single hybridoma sample were co-expressed in HEK293-6E cells (National Research Council) in all possible combinations to determine the correct heavy and light chain pairing. Antibodies produced were assayed for binding to antigen that was transiently expressed on HEK293 cells.

### Results

Of the 24 antibodies initially identified as binding to human 4-1BB, a total of 18 paired antibody VH and VL sequences (shown in Table 13) were obtained from the hybridomas and cloned into the pTT5 vector as human-mouse chimeric antibodies, with mouse VH and VL domains and a human IG1 Fc. Mouse VH domains were cloned in frame with a human CH1-hinge-CH2-CH3 construct, and mouse VL domains cloned in frame with the human kappa CL domain.

### Expression of 4-1BB Chimeric Antibodies

The 18 chimeric mouse-human 4-1BB antibodies were produced by transfection of two plasmids into HEK293-6E cells, one plasmid containing the heavy chain and the other plasmid containing the light chain.

HEK293-6E cells were split 1:10 72 hours prior to transfection to ensure growth-phase cells. These cells were then counted and resuspended at 10⁶ cellsml⁻¹ in OptiMEM™ (Thermofisher). A transfection mix was made by mixing 30 μl 293fectin™ (Thermofisher) and 1.5 ml OptiMEM™. This mix was then incubated at room temperature. After five minutes, 1. 5ml OptiMEM™ and 15 μg of each of the plasmids containing the antibody heavy or light chains in the pTT5 vector were added. This mix was then left at room temperature for 20 minutes, and then added dropwise to cells, with a total volume of 3 ml. Cells were then left at 37C in a 5% CO2 atmosphere in a shaking incubator at 120 rpm for five days.

In some cases, the antibody levels within the supernatant were quantified using an Octet™ RED96 (ForteBio) with a Protein A tip, and the supernatants used immediately in assays.

The supernatants were also purified using Protein A. To purify the antibodies, cells were first removed from the antibody supernatants by centrifuging at 1000 rcf for 15 minutes. Protein A Gravitrap™ columns were then prepared by equilibration using 10 ml PBS, followed by application of antibody supernatant in batches of 10ml. Once all of the supernatant had flowed through the column, the column was washed twice, each time with 10 ml PBS. Elution of the antibody was performed by the addition of 3 ml 0.1IM glycine-HCl pH 2.7. The eluted antibody sample was then neutralized using 1M Tris-HCl, pH 9.

To concentrate the antibody sample and perform a buffer exchange, the antibody samples were loaded into a Vivaspin™ 30 kDa MWCO protein concentrator spin column (GE Healthcare). The columns were then spun at 3000 rcf for 7 minutes to concentrate the antibody. 4 ml PBS was then added to the column to exchange buffers, and the column was then spun again, to exchange buffers into PBS. The antibody levels in the resulting solution were then measured using 260 nm/280 nm absorbance ratio using a Nanodrop™ Spectrophotometer (Thermofisher).

### Example 7

### Activity of Chimeric 4-1BB Antibodies in 4-1BB NF-kB-Luciferase Reporter Assay

To test the ability of chimeric mouse-human 4-1BB antibodies to stimulate 4-1BB activation and downstream signaling, a reporter gene assay was used. The cells used in this experiment produce luciferase under the control of the NF-kB promoter when signaling is induced by ligation of the 4-1BB receptor.

This experiment was set up in a manner similar to that described in Example 3, except that antibody supernatants were used in place of bispecific antibodies, and no tumour cells were used. The antibody supernatants were diluted in Assay Buffer (RPMI (Gibco)/1% FBS (Gibco)) to 5000 ng/ml, 1666 ng/ml, 554 ng/ml and 184 ng/ml. Rabbit-anti-human IgG Fc (Thermofisher) polyclonal secondary antibody was then added to a concentration of 15000 ng/ml and the antibody mixture left at room temperature.

After 45 minutes, 30 μl of the antibody mix was added to wells. If the concentration of antibody in the supernatant was below 5000 ng/ml, supernatants were diluted from neat (v20023, v20025, v20028, v22033. v22034). As a positive control, v12592 was diluted either in supernatant (ESN) or RPMI. The negative control was v16992 diluted in ESN. 4-1BB Thaw-and-use Jurkat cells (Promega) were then added, followed by a 5 hour incubation and then Bio-Glo™ reagent (Promega) was added, as described in Example 3. The data was acquired and analyzed as in Example 3.

### Results

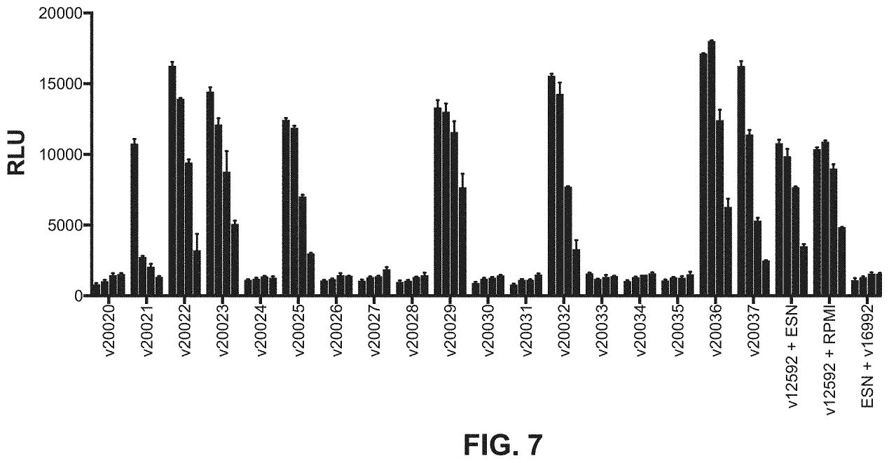

Eight antibodies induced the production of luciferase: v20021, v20022, v20023, v20025, v20029, v20032, v20036, v20037 suggesting that they agonise 4-1BB (FIG. 7). These antibodies were then purified from supernatant and taken forward to assess which 4-1.13B domains they bound to.

### Example 8

### Determination of 4-1BB Domain Binding

To determine which domains of human 4-1BB the chimeric antibodies recognized, the chimeric antibodies were tested for binding to human 4-1BB, dog 4-1BB and a chimeric human-dog 4-1BB protein. The human-dog 4-1BB protein included a set of mutations within domain 4 that modified human 4-1BB to dog.

### Preparation of Human, Dog and Human-Dog 4-1BB

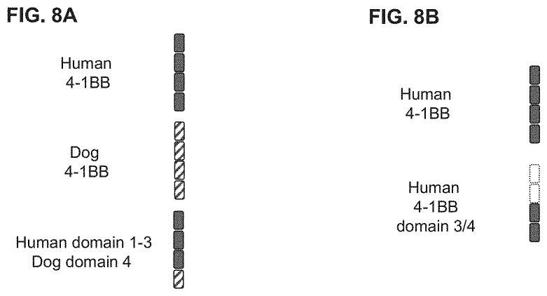

To generate expression constructs for soluble human, dog and human-dog chimeric 4-1BB, synthesized DNA having 4-1BB ECD-TEV-IgG1 hinge-CH2-CH3-10× His was made. Table 4 below provides the sequences of these constructs and FIG. 8A provides a representation of the 4-1BB portion of these constructs, Both dog and human 4-1BB extracellular domain included residues 24-186 of the 4-1BB protein sequence taken from Uniprot (IDs E2R1R9 and Q0107011 for dog and human 4-1BB, respectively). The mutations in the human-dog chimera to mimic dog 4-1BB in domain 4 were K115Q, C121R, R134Q, R154S and V156A (described in WO2012/032433).

TABLE 4
4-1BB domain-binding constructs

| | SEQ | |
|---|---|---|
| | ID | |
| Name | NO: | Sequence |
| 4-1BB | 39 | LQDPCSNCPAGTFCDNNRNQICSPCPNSFSSAGGQRTCDICRQCK |
| human | | GVFRTRKECSSTSNAECDCTPGFHCLGAGCSMCEQDCKQGQELTK |
| | | KGCKDCCFGTFNDQKRGICRPWTNCSLDGKSVLVNGTKERDVVCG |
| | | PSPADLSPGASSVTPPAPAREPGHSPQENLYFQSPKSCDKTHTCP |
| | | PCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGK |
| | | EYKCKVSNKALPAPIEKTISKAKGQPREPQVYTLPPSRDELTKNQ |
| | | VSLTCLVKGFYPSDIAVEWESNGQPENNYKTTPPVLDSDGSFFLY |
| | | SKLTVDKSRWQQGNVFSCSVMHEALHNHYTQKSLSLSPGKGGHHH |
| | | HHHHHHH |
| 4-1BB | 40 | LQDPCSNCPAGTFCDNNRNQICSPCPPNSFSSAGGQRTCDICRQC |
| dog- | | KGVFRTRKECSSTSNAECDCTPGFHCLGAGCSMCEQDCKQGQELT |
| human | | KQGCKDCRFGTFNDQKRGICQPWTNCSLDGKSVLVNGTKESDAVC |
| | | GPSPADLSPGASSVTPPAPAREPGHSPQENLYFQSPKSCDKTHTC |
| | | PPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNG |
| | | KEYKCKVSNKALPAPIEKTISKAKGQPREPQVYTLPPSRDELTKN |
| | | QVSLTCLVKGFYPSDIAVEWESNGQPENNYKTTPPVLDSDGSFFL |
| | | YSKLTVDKSRWQQGNVFSCSVMHEALHNHYTQKSLSLSPGKGGHH |
| | | HHHHHHHH |
| 4-1BB | 41 | IQDSCSKCPAGTFCGKNKSQICIPCPPNSFSSTSGQKACDICRQC |
| dog | | EGVFRTKKVCSPISNAECECISGFHCLGAGCTMCEQDCKQGQELT |
| | | KQGSCKDCRFGTFNDQKHGICQPWTNCSLDGKSVLVNGTKESDAV |
| | | CGPASAGFSPGTASATTPAPARDPGHTSQENLYFQSPKSCDKTHT |
| | | CPPCPAPELLGGPSVFLFPPKPKDTLMISRTPEVTCVVVDVSHED |
| | | PEVKFNWYVDGVEVHNAKTKPREEQYNSTYRVVSVLTVLHQDWLN |
| | | GKEYKCKVSNKALPAPIEKTISKAKGQPPEPQVYTLPPSRDELTK |
| | | NQVSLTCLVKGFYPSDIAVEWESNGQPENNYKTTPPVLDSDGSFF |
| | | LYSKLTVDKSRWQQGNVFSCSVMHEALHNHYTQKSLSLSPGKGGH |
| | | HHHHHHHHH |

The pTT5 vectors containing the 4-1BB expression constructs were then produced and purified in a manner similar to the antibodies in Example 1, except a 500 ml culture volume was used. Caliper results indicated that human, dog and chimeric human-dog, 4-1BB constructs were prepared in substantially pure form.

### Domain-Mapping of 4-1BB Antibodies by ELISA

A soluble antigen binding ELISA was performed to assess antibodies for their ability to bind outside of 4-1BB domain 4. The goal was to determine if samples have differential binding to hybrid or chimeric human-dog 4-1BB versus human 4-1BB to suggest whether or not binding is outside of domain 4. However, if the tested antibodies were able to bind to dog 4-1BB assessment of binding to domain 4 by this method would not be accomplished.

Soluble human, dog or human-dog 4-1BB-Fc proteins were prepared in PBS pH 7.4 (Thermo Fisher, Whetham, Mass.) at 400 ng/mL. 4-1BB-Fc proteins were added at 50 μL/well to wells of a 96-well flat bottom ELISA plates (Corning 3368). The plates were covered by a lid, sealed with parafilm, and left overnight at 4° C. The next day, the plates were washed three times with 300 μL/well distilled water using a BioTek 405 HT microplate washer (BioTek, Winooski, Vt.) and tapped to dry. Wells were then blocked by adding 200 μL/well blocking buffer (2% w/v skim milk powder in PBS) and left at room temperature for 1 hour. The plates were washed as previously and tapped to dry. Antibody samples were then diluted in assay buffer (2% w/v skim milk powder in PBS) to 10 μg/mL final or used neat if samples were below 10 μg/mL. Directly in assay plates, samples were serially diluted five times 1:8 in duplicate in assay buffer with a final volume of 50 μL/well. Similarly, control antibodies were prepared and diluted in assay buffer. For wells containing no antibody sample, assay buffer was added at 50 μL/well. The plates were then covered with a lid, sealed with parafilm, and incubated overnight at 4° C. The next day, plates were washed with the plate washer as previously and tapped dry. For detection of sample binding to soluble antigen, Peroxidase AffiniPure goat anti-human F(ab′)₂ (Jackson ImmunoResearch, West Grove, Pa.) was prepared in assay buffer at 0.4 μg/mL. For detection of coated antigen, Peroxidase AffiniPure goat anti-human Fc (Jackson ImmunoResearch, West Grove, Pa.) was prepared in assay buffer at 1 μg/mL. Both secondaries were added at 50 μL./well and plates were incubated at room temperature for 30 minutes. The plates were washed and dried as previous and TMB substrate (Cell Signaling Technology, Danvers, Mass.) was added at 50 μL/well. After incubation at room temperature for ten minutes, the reaction was neutralized with 1M HCl (VWR, Radnor, Pa.). The plate absorbance at OD450 was scanned on the BioTek Synergy™ H1 plate reader (BioTek, Winooski, Vt.).

### Results

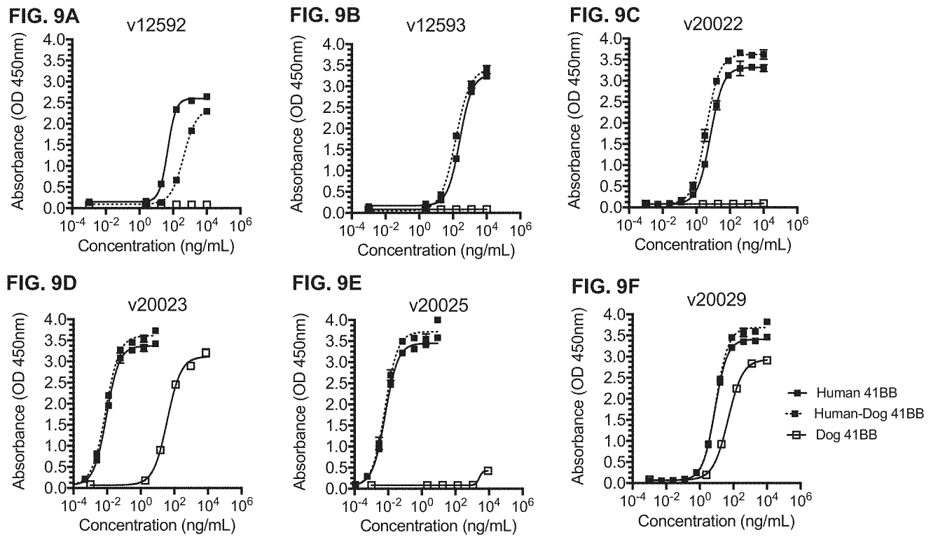

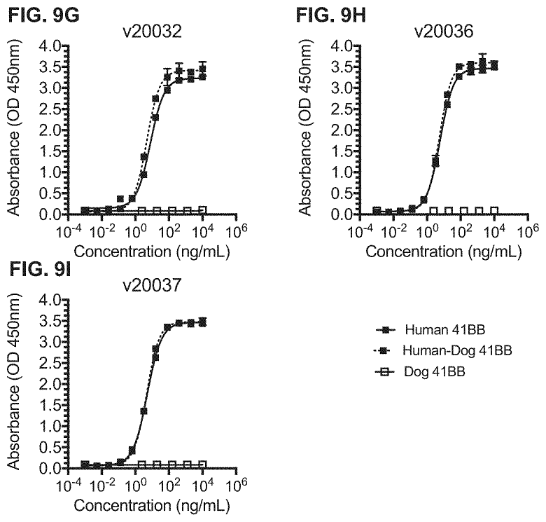

As shown in FIGS. 9A to FIG. 9I, all chimeric antibodies tested were able to bind human 4-1BB. However, v20023 and v20029 also bound dog 4-1BB, suggesting that the domain binding of those two antibodies cannot be assessed by this method. The remaining antibodies did not bind dog 4-1BB.

v20022, 20025, v20032, v20036 and v20037 showed equal binding on human and human-dog chimeric 4-1BB, suggesting that all these antibodies bound outside domain 4 (amino acids 115-156). MOR7480.1-IG1 (variant 12592), as expected, showed a reduction of binding to dog-human chimeric 4-1BB compared to human 4-1BB, suggesting that its binding domain is within the amino acids 115-156. v12593, a version of Urelumab with an IgG1 Fc, similar to the tested antibodies, bound human and human-dog chimeric 4-1BB equally, suggesting that its binding domain also lies outside amino acids 115-156. v20027 did not show binding in this experiment and was excluded from future analysis.

### Domain Binding of Antibodies Using Truncated 4-1BB Proteins

As some of the antibodies were cross-reactive to dog 4-1BB, another method was required to determine to which domain antibodies bound. As an alternative, truncated transmembrane 4-1BB constructs were made, where 4-1BB domains 3 and 4 only would be expressed. FIG. 8B provides a representation of the truncated transmembrane 4-1BB constructs that were made.

Construction of4-1BB Domain Vectors

Constructs were synthesized having either full-length human 4-1BB (residues 24-255) or extracellular domains 3 and 4 (residues 86-255) along with the native human transmembrane and intracellular parts of 4-1BB. Full-length mouse 4-1BB was also cloned. All vectors also contained the native signal peptide (MGNSCYNIVATLLLVLNFERTRS, SE ID NO:42) and were run through Signal 4.1 (www.cbs.dtu.dk/services/SignalP/) to predict the successful cleavage of the signal peptide. All constructs were synthesized in the form 3′-EcoRI-4-1BB-BamHI-5′ and cloned into an EcoRI-BamHI digested pTT5 vector.

To test binding of antibodies to 4-1BB, 293E6 cells were transfected as before, except that all human 4-1BB constructs were co-transfected with mouse 4-1BB, to act as a carrier protein. Twenty-four hours after transfection, 2×10⁵ cells were labelled with 2.5 μg of antibody for one hour on ice, and then analysed by flow cytometry using a Attune cytometer (ThermoFisher, Waltham, Mass., U.S.). The antibodies had been pre-complexed with Zenon-Alexa-647 reagent (ThermoFisher) using manufacturer's instructions.

### Results

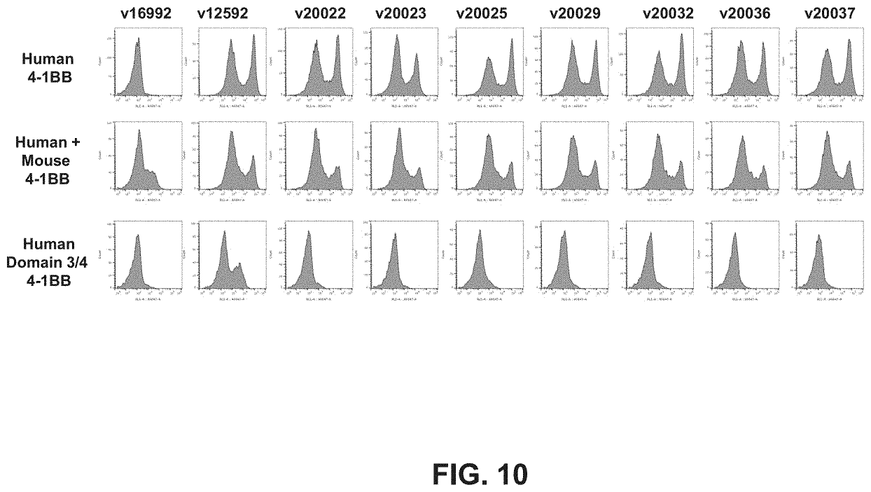

The results of this experiment are shown in FIG. 10, All antibodies showed binding to cells transfected with human 4-1BB and to cells transfected with human and mouse 4-1BB. Variant 16992, an anti-RSV antibody, was used as a negative control. As expected, v12592 showed binding to cells transfected with the 4-1BB domain 3 and 4 only construct (amino acids 86-255), as its hypothesized binding domain is between amino acids 115 and 156 (domain 4). v20022, v20023, v20025, v20029, v20032, v20036 and v20037 antibodies did not show binding to cells transfected with the domain 3 and 4 only construct, suggesting that all of the antibodies bind outside of those domains, and bind to an epitope at least partially within amino acids 24-85 of the mature 4-1BB protein. This data reinforces the conclusions of the human-dog chimera experiment, that v20022, v20025, v20029, v20036 and v20037 do not bind domain 4.

### Example 9

### Binding of Chimeric Anti-4-1BB Antibodies to Cynomolgus and Mouse 4-1BB

To assess the binding of v20022, v20023, v20025, v20029, v20032, v20036 and v20037 to native transmembrane Cynomolgus (Macaca fascicularis) and Mouse (Mus musculus) 4-1BB, a homogeneous cell binding assay was performed using the CellInsight CX5 platform (Thermo Fisher, Watham, Mass.). This experiment used cells transiently expressing either Cynomolgus or mouse 4-1BB.

To prepare cells for transfection, suspension HEK293-6e cells (National Research Council Canada, Montreal, QB) were cultured in 293 Freestyle Media (ThermoFisher, Waltham, Mass.), with 1% FBS (Corning, Corning, N.Y.) in 250 mL Erlenmeyer flasks (Corning, Corning, N.Y.) at 37 ° C., 5% CO2 in a humidified incubator with rotation at 115 rpm. Before transfection, HEK293-6e cells were re-suspended to 1×10⁶ cells/mL, in fresh 293 Freestyle media. Cells were then transfected using 293fectin™ transfection reagent (Thermo Fisher, Watham, Mass.) at a ratio of 1 μg DNA110⁶ cells in Opti-MEM™ Reduced Serum Medium (Thermo Fisher, Watham, Mass.). Cells were transfected with pTT5 DNA vectors containing either full length cynomolgus monkey 4-1BB with a flag-tag (CL#11070 SEQ ID NO:43), mouse 4-1BB-flag (CL#11063 SEQ ID NO:44) as shown in Table 5, or vector containing GFP as a control for transfection efficiency. The cells were incubated for 24 hours at 37° C., 5% CO2 in a humidified incubator with rotation at 115 rpm.

TABLE 5
Cynomolgus or mouse 4-1BB sequences

| | SEQ | |
|---|---|---|
| | ID | |
| Name | NO: | Sequence |
| Cyno 4- | | MGNSCYNIVATLLLVLNFERTRSLQDLCSNCPAGTFCDNN |
| 1BB- | 43 | RSQICSPCPPNSFSSAGGQRTCDICRQCKGVFKTRKECSS |
| Flag | | TSNAECDCISGYHCLGAECSMCEQDCKQGQELTKKGCKDC |
| | | CFGTFNDQKRGICRPWTNCSLDGKSVLVNGTKERDVVCGP |
| | | SPADLSPGASSATPPAPAREPGHSPQIIFFLALTSTVVLF |
| | | LLFFLVLDYKDDDDK |
| Mouse | 44 | MGNNCYNVVVIVLLLVGCEKVGAVQNSCDNCQPGTFCRKY |
| 4-1BB- | | NPVCKSCPPSTFSSIGGQPNCNICRVCAGYFRFKKFCSST |
| Flag | | HNAECECIEGFHCLGPQCTRCEKDCRPGQELTKQGCKTCS |
| | | LGTFNDQNGTGVCRPWTNCSLDGRSVLKTGTTEKDVVCGP |
| | | PVVSFSPSTTISVTPEGGPGGHSLQVLTLFLALTSALLLA |
| | | LIFITLLFDYKDDDDK |

Antibody samples were prepared at concentrations of neat, 1:4 and 1:16 final in PBS pH 7.4 (Thermo Fisher, Watham, Mass.)+2% FBS in Eppendorf tubes and 30 μl of antibody mix was added to wells of a 384-well black optical bottom plate (ThermoFisher). v12592 was used as a positive control for binding to Cynomolgus 4-1BB, and human IgG1 as a negative control. Anti-mouse 4-1BB antibody LOB12.3 (BioXCell, West Lebanon, N.H.) and its respective rat IgG1 isotype control (R&D Systems, Minneapolis, Minn.) were used as controls for mouse binding. A cell mixture of transfected HEK293-6e cells (10,000 cells per 30 μL), Vybrant™ DyeCycle™ Violet nuclear stain at 2 μM final (Thermo Fisher) and Goat anti Human IgG Fc A647 at 0.6 μg/mL (Jackson ImmunoResearch, Westgrove, Pa.) was prepared. The cells were vortexed briefly to mix added to wells at 30 μL/well. The plate was incubated at room temperature for 3 hours before scanning. Data analysis was performed on the CellInsight CXS with the HCS high content screening platform (ThermoFisher), using BioApplication “CellViability” with a 10× objective. Samples were scanned on the 385 nm channel to visualize nuclear staining and 650 nm channel to assess cell binding. The mean object average fluorescence intensity of A647 was measured on channel 2 to determine binding intensity. This intensity was then divided by the intensity of staining seen in the GFP-transfected wells to give a fold binding induced by the antibody.

### Results

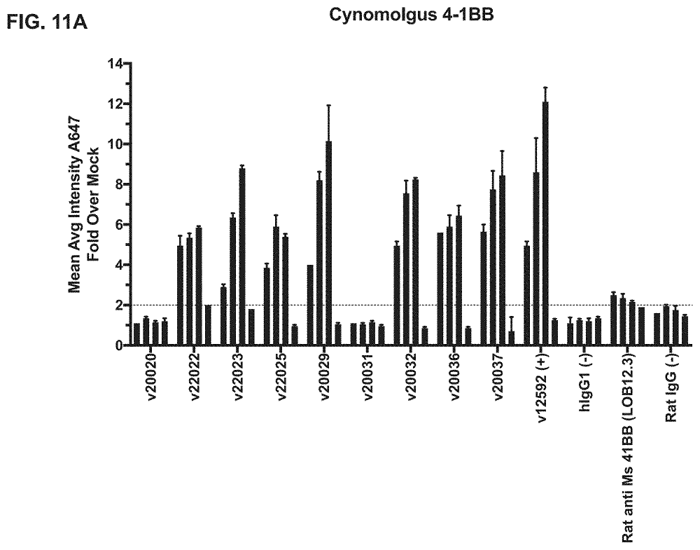

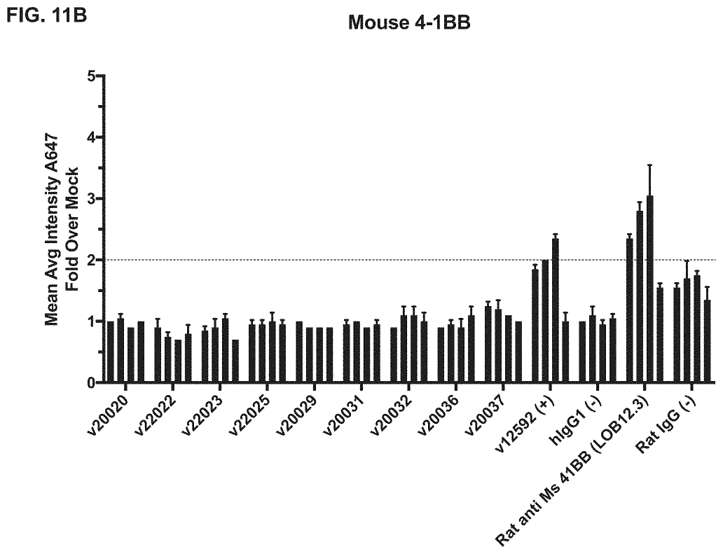

All antibodies with the exception of v20020 (1B2) and v20031 (4B1) appeared to bind Cynomolgus 4-1BB (FIG. 11A). v12952 was used as a positive control for cyno 4-1BB binding, with higG1 being its matched isotype control, which does not show binding. No antibodies bound mouse 4-1BB (FIG. 11B). LOB12.3 was used as a positive control for binding to mouse 4-1BB, with the Rat IgG being a matched isotype control, which does not show binding. cl Example 10

### Humanization of Mouse 5G8, 1G1 and 1C8 VH and NT Sequences

Humanized versions of three of the mouse anti-human 4-1BB antibodies generated in Example 6 were prepared as described below.

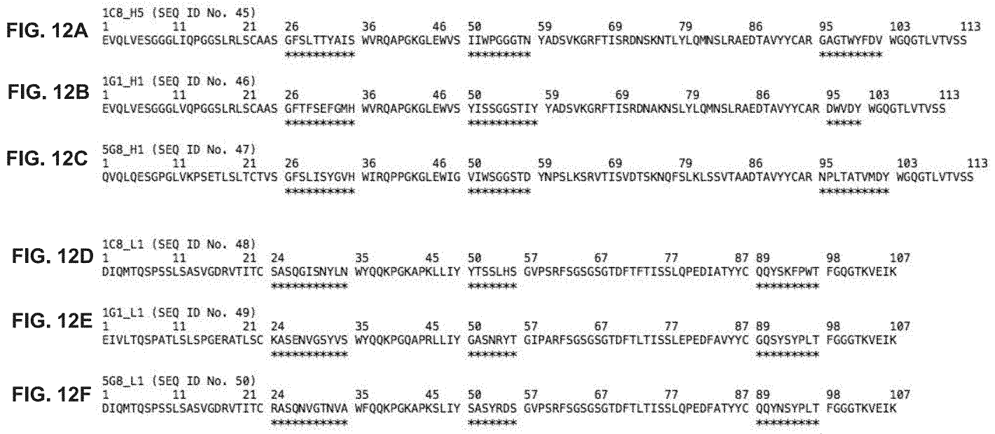

Humanization of mouse 1C8, 1G-1 and 5G8 variable light (VL) and variable heavy (VH) domains was performed as follows. Sequence alignment of mouse 1C8 VH and VL sequences to respective human germlines identified IGHV3-66*03 and IGKV1D-33*01 among the closest as well as relatively frequent germlines in humans. Sequence alignment of mouse 1G1 VH and VL sequences to respective human germlines identified IGHV3-48*03 and IGKV3-11.*01 among the closest, as well as relatively frequent germlines in humans. Sequence alignment of mouse 5G8 VH and VL sequences to respective human germlines identified IGHV4-59*08 and IGKV1-16*01 among the closest, as well as relatively frequent germlines in humans. CDRs, identified according to AbM definition (see Table A), were ported onto the framework of these selected human germlines. FIG. 12 provides the sequences of the resulting VH (FIGS. 12 A-C) and VL sequences (FIG. 12 D-F). Back mutations to mouse residues in such generated sequence, at positions judged to likely be important for the retention of binding affinity to human 4-1BB, were included in such way to create several humanized sequences in which next one builds on the previous one, and where the first humanized sequence contains minimal number of back mutations or no back mutations.

For 1C8 this process led to four variable heavy chain humanized sequences and three variable light chain humanized sequences. For 1G1 this process led to three variable heavy chain humanized sequences and four variable light chain humanized sequences. For 5G8 this process led to four variable heavy chain humanized sequences and four variable light chain humanized sequences. Full heavy chain sequence containing humanized heavy chain variable domain (VH) and higG1 heavy chain constant domains (CH1, hinge, CH2, CH3), as well as full light chain sequence containing humanized light chain variable domain (VL) and human kappa light chain constant domain (kappa CL) were created for 1C8, 1G1 and 5G8. Antibodies were then assembled such that each humanized heavy chain was paired with each of the humanized light chains, making a total of twelve humanized variants each for 1C8 and 1G1, and 16 variants for 5G8, (Table 6). The amino acid sequences for each of the humanized heavy chains and humanized light chains are provided in Table 14.

TABLE 6
Humanized 1C8, 1G1 and 5G8 variants and their composition.

| | Composition |
|---|---|
| antibody Variant | HL |
| v20022 1C8 mouse-human | |
| parental chimera | |
| v28717 | H5L1 |
| v28719 | H5L2 |
| v28720 | H5L3 |
| v28721 | H6L1 |
| v28722 | H6L2 |
| v28723 | H6L3 |
| v28724 | H7L1 |
| v28725 | H7L2 |
| v28726 | H7L3 |
| v28727 | H8L1 |
| v28728 | H8L2 |
| v28730 | H8L3 |
| v20023 1G1 mouse-human | |
| parental chimera | |
| v28683 | H1L1 |
| v28684 | H1L2 |
| v28685 | H1L3 |
| v28686 | H1L4 |
| v28687 | H2L1 |
| v28688 | H2L2 |
| v28689 | H2L3 |
| v28690 | H2L4 |
| v28691 | H3L1 |
| v28692 | H3L2 |
| v28693 | H3L3 |
| v28694 | H3L4 |
| v20036 5G8 mouse-human | |
| parental chimera | |
| v28695 | H1L1 |
| v28696 | H1L2 |
| v28697 | H1L3 |
| v28698 | H1L4 |
| v28699 | H2L1 |
| v28700 | H2L2 |
| v28701 | H2L3 |
| v28702 | H2L4 |
| v28703 | H3L1 |
| v28704 | H3L2 |
| v28705 | H3L3 |
| v28706 | H3L4 |
| v28707 | H4L1 |
| v28711 | H4L2 |
| v28712 | H4L3 |
| v28713 | H4L4 |

### Production of Humanized Antibodies

Each of the humanized 1C8, 1G1 and 5G8 VH and VL sequences described in Table 6 as well as the parental mouse VH and VL sequences were used to prepare humanized antibodies in the naturally occurring, or FSA antibody format, containing two identical full-length heavy chains and two identical kappa light chains. Table X identifies the clones that make up each of the antibody constructs. The polypeptide sequences of each clone can be found in Table Y.

Each of the humanized VH domain sequences (SEQ ID NOs:45, 46, 47, 51, 52, 53, 56, 57, 61, 62, and 63) was appended to the human CH1-hinge-CH2-CH13 domain sequence of IGHG1*01 (SEQ ID NO:68) to obtain a protein sequence for four humanized 1C8, three humanized 1G1 and four humanized 5G8 full heavy chain sequences. Each of the humanized VL domain sequences (SEQ ID NOs:48, 49, 50, 54, 55, 58, 59, 60, 64, 65, and 66) was appended to the human kappa CL sequence of IGKC*01 (SEQ ID NO:67) to obtain a protein sequence for three humanized 1C8, four humanized 1G1 and four humanized 5G8 full light chain sequences. In a similar manner, 1C8, 1G1 and 5G8 mouse-human parental antibody chimera heavy and light chain sequences were assembled, with the difference that variable domain sequences were mouse (SEQ ID Nils:7, 9, 35 (VH) and 8, 10, 36 (VL)) and constant domain sequences were human (SEQ ID NOs:68 and 67 correspondingly). These sequences were reverse translated to DNA, codon optimized for mammalian expression, and gene synthesized.

All mouse-human parental and humanized full heavy and full light chain sequences were preceded by a signal peptide which is an artificially designed sequence MRPTWAWWLFLVLLLALWAPARG [SEQ NO:1] (ref: Barash S et al., Biochem and Biophys Res. Comm. 2002; 294, 835-842). For all parental and humanized heavy and light chains, vector inserts were prepared as described in Example 1 and cloned into pTT5 to produce expression vectors.

The heavy and light chains of antibody variants were expressed in 100 mL CHO cultures and purified as described in Example 1. Following protein-A purification, purity of samples was assessed by non-reducing and reducing High Throughput Protein Express assay as described in Example 1.

Post protein-A purification, samples were either buffer exchanged into DPBS and aseptically filtered or, depending on their homogeneity assessed by UPLC-SEC, subjected to SEC purification as described in Example 1.

### Results

Yields of protein post protein-A purification were in the range of ˜3.5-9 mg for humanized 1C8 variants, ˜4.5-9.5 mg for humanized 1G1 variants and ˜4.5-8 mg for humanized 5GS variants. Non-reducing and reducing LabChip post protein-A reflected single species corresponding to full size antibody and intact heavy and light chains in all cases (data not shown). Endotoxin levels were within the specifications.

### Example 11

### Biophysical Assessment of Purified Humanized 1C8, 1G1 and 5G8 Antibodies

Samples of humanized antibody variants were subjected to UPLC-SEC in order to assess species homogeneity following protein-A purification. UPLC-SEC was performed as described in Example 1.

### Results

UPLC-SEC analysis of protein A purified humanized 1C8 antibody variants was reflective of high species homogeneity in the case of variants 28717. 28719, 28720, 28721 (data not shown). UPLC-SEC profiles of all other humanized 1C8 variants (data not shown) reflected good homogeneity, as judged by the presence of small peaks (likely reflective of aggregates) and shoulder to the main peak (possibly reflective of different antibody conformation).

UPLC-SEC analysis of protein A purified humanized 1G1 antibody variants was reflective of high species homogeneity for variants 28683, 28684, 28685 and 28686. UPLC-SEC profiles of all other humanized 1G1 variants reflected slightly lower homogeneity (data not shown).

UPLC-SEC analysis of protein A purified humanized 5G8 antibody variants showed good species homogeneity for all variants. UPLC-SEC analysis was repeated for final pools of samples post SEC purification and these samples showed homogeneity in the range of 99.2-100.0% (data not shown).

### Example 12

### Binding of Humanized 1C8, 1G1 and 5G8 Antibodies to Human 4-1BB by SPR

To compare the ability a the humanized antibodies to bind human 4-1BB, the affinity of humanized antibodies was compared to the parental chimeric antibodies by Surface Plasmon Resonance (SPR).

Protein material post protein-A or SEC was assessed for binding to human 4-1BB. The antigen-binding affinity was determined by SPR as described in Example 2.

### Results

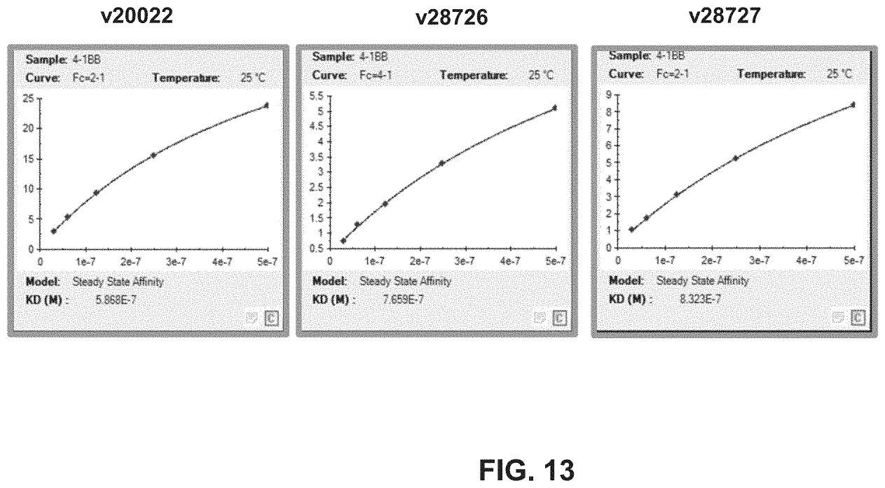

As can be seen from Table 7 (shown in Example 13), SPR binding assay performed on the humanized 1C8 variants revealed that four of the humanized 1C8 antibody variants (v28726, v28727, v28728, v28730) bound h4-1BB with comparable affinity to the parental chimera antibody (variant v20022) and eight variants did not bind h4-1BB. These variants have in common humanized 1C8 heavy chains H5 and H6 in combination with humanized 1C8 light chains L1, L2 or L3. Humanized IC8 light chains L1 and L2 also do not bind 4-1BB when in combination with humanized 1C8 heavy chain H7. These results suggest that back mutations to mouse residues at specific positions incorporated in humanized 1C8 heavy chain H8 as well as into humanized 1C8 light chain L3 are important to maintain CDR conformations such that binding to h4-1BB can be retained. FIG. 13 provides SPR sensorgrams for the parental chimera and representative humanized variants that were able to bind human 4-1BB.

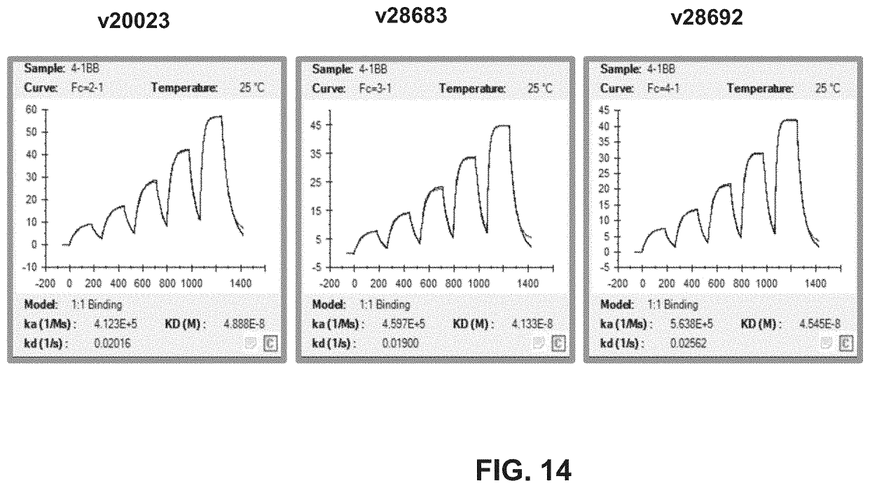

As can be seen from Table 7, SPR binding assay performed on the humanized 1G1 variants revealed that all humanized 1G-1 antibody variants bound h4-1BB with affinity within 2-fold of the KD of the parental chimera antibody (variant v20023). This suggests that the frameworks of humanized 1G1 heavy and light chain without back mutations to mouse residues are sufficient to maintain the CDR conformations necessary for binding to h4-1BB, FIG. 14 provides SPR sensorgrams for the parental chimera and representative humanized variants that were able to bind human 4-1BB.

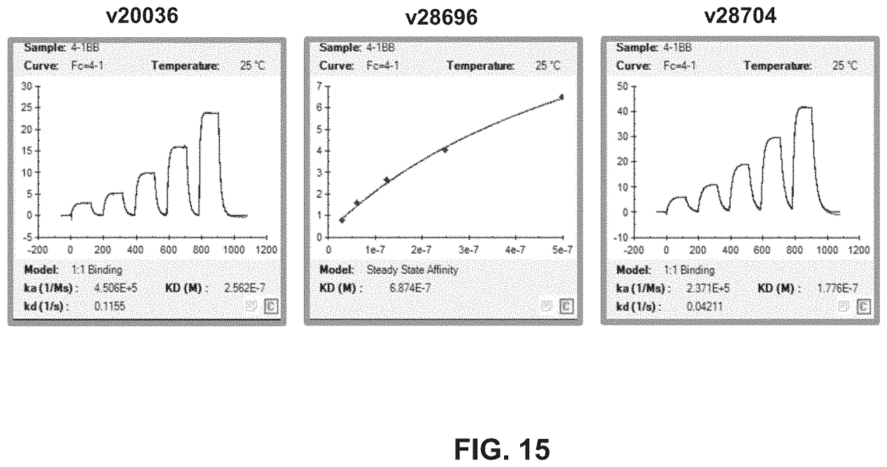

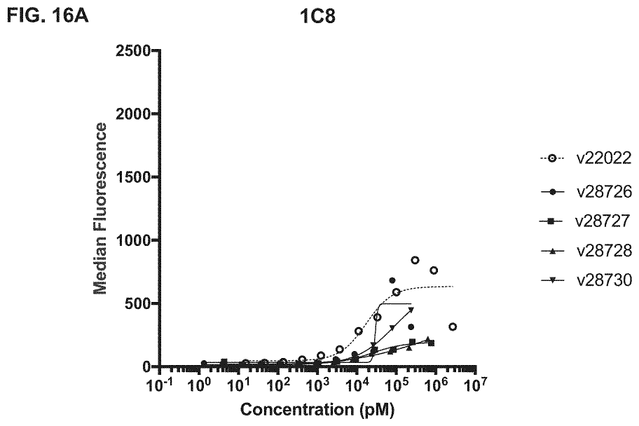

SPR binding assay performed on the humanized 5G8 variants showed, as can be seen in Table 7 and FIG. 15, that seven humanized 5G8 antibody variants (v28700, v28704, v28705, v28706, v28711, v28712, v28713) bound h4-1BB with affinity within 2-fold of the KD of the parental chimera antibody (variant v20036). Seven humanized 5G8 variants bound h4-1BB with 2-3× decreased affinity compared to the KD of the parental chimera antibody (v28696, v28697, v28698, v28701, v28702, v28703, v28707) and two variants did not bind h4-1BB. These two variants have in common humanized light chain L1. The seven 5G8 variants with slightly decreased affinity to 4-1BB have in common humanized heavy chain H1 or H2, or humanized heavy chain H3 or H4 in combination with humanized light chain 1. The results for humanized 5G8 antibody suggest that the back mutations to mouse residues at specific positions incorporated into L2, as well as into H3 are necessary to maintain the required CDR conformations to bind h4-1BB with a KD comparable to the parental chimera variant v20036.

### Example 13

### Comparison of Binding of Humanized Variants by Flow Cytometry

To examine binding of antibodies to native cell-surface expressed 4-1BB, a flow cytometry binding assay was carried out as described below.

Jurkat T cells engineered to stably express human 4-1BB were used to measure binding of antibodies to human 4-1BB. Antibodies were diluted in 5 μl FB (PBS 2% FCS) 1:3 from stock in wells of a 96 V-well plate, and cells added on top. The cells were then left on ice for 30 minutes for the antibodies to bind. The cells were then washed twice in FB, and then incubated in 50 μl FB containing 2 μg/ml goat anti-human Alexa647 antibody (Jackson Immunoresearch). The cells were then left on ice for a further 20 minutes, washed twice in FB, resuspended in 100 μl FB and analysed on a BD Fortessa™ X20. The subsequent data files were analysed using FlowJo™ and Prism 7 (GraphPad) using a four-parameter non-linear regression.

### Results

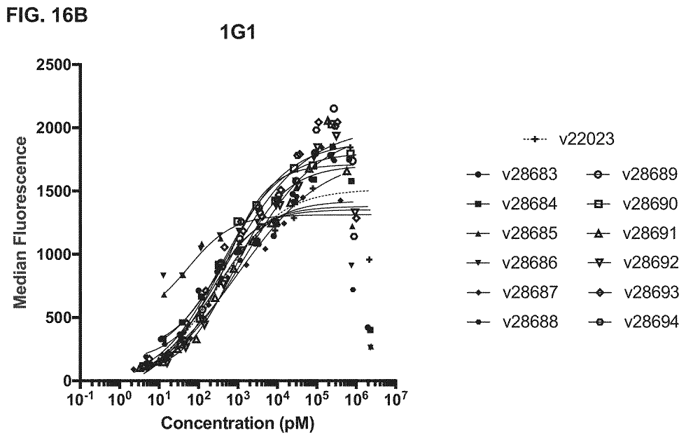

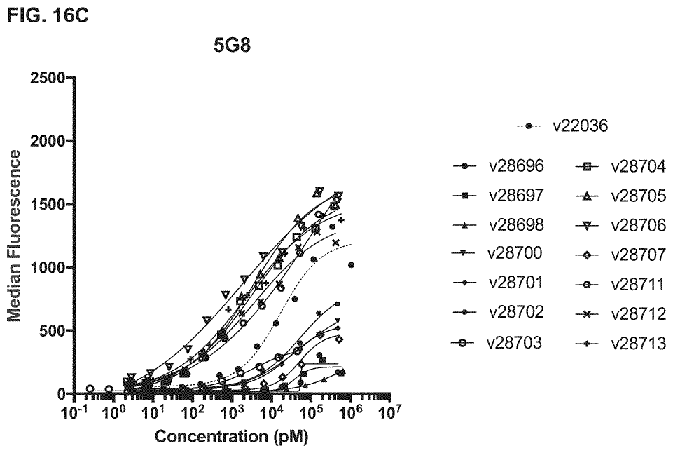

FIG. 16A, 16B and 16C depict the ability of the 1C8, 1G1 and 5G8 humanized antibodies, respectively, to bind to 4-1BB-expressing Jurkat T cells. Similar to the SPR results, antibodies which derive from the 1C8 paratope bound poorly, including the parental antibody, v20022. The original mouse 1G1 paratope, v22023, bound well to 4-1BB as would be expected from the SPR results. The humanized antibodies based on the 1G1 paratope also bound well, and there was little drop in binding seen as a result of humanization. Similarly, 5G8 antibodies also bound well, with some antibodies displaying greater binding to 4-1BB when compared to the parental mouse-human chimeric antibody v22036.

The results of the SPR and flow cytometry assays are summarized in Table 7 below.

TABLE 7
Antigen binding assessment of the humanized antibody variants by SPR

| | SPR | | Flow Cytometry | |
|---|---|---|---|---|
| | Mean KD | Std Dev | EC50 | EC50 |
| Variant | (M) n = 3 | KD (M) | (M) | Std Error (M) |
| v20022 1C8 mouse-human | 5.82E−07 | 1.38E−08 | 1.7298E−08 | 2.0338E−12 |
| parental chimera | | | | |
| v28717 | DNB | — | ND | — |
| v28719 | DNB | — | ND | — |
| v28720 | DNB | — | ND | — |
| v28721 | DNB | — | ND | — |
| v28722 | DNB | — | ND | — |
| v28723 | DNB | — | ND | — |
| v28724 | DNB | — | ND | — |
| v28725 | DNB | — | ND | — |
| v28726 | 7.63E−07 | 5.91E−08 | NF | NF |
| v28727 | 8.13E−07 | 3.59E−08 | 2.42E−08 | 1.40E−12 |
| v28728 | 1.42E−06 | 8.02E−08 | 7.13E−07 | 1.38E−11 |
| v28730 | 7.62E−07 | 4.69E−08 | 8.87E−08 | 1.12E−12 |
| v20023 1G1 mouse-human | 4.96E−08 | 8.08E−10 | 3.37E−10 | 8.69E−12 |
| parental chimera | | | | |
| v28683 | 4.23E−08 | 1.54E−09 | 2.23E−10 | 2.60E−11 |
| v28684 | 3.85E−08 | 9.29E−10 | 2.47E−10 | 1.78E−11 |
| v28685 | 3.12E−08 | 0 | 5.51E−11 | 1.71E−08 |
| v28686 | 3.11E−08 | 5.77E−11 | NF | NF |
| v28687 | 4.03E−08 | 3.51E−10 | 1.00E−09 | 2.39E−12 |
| v28688 | 6.92E−08 | 1.34E−09 | 3.60E−10 | 5.21E−12 |
| v28689 | 5.83E−08 | 5.69E−10 | 4.86E−10 | 2.32E−12 |
| v28690 | 3.44E−08 | 3.51E−10 | 3.76E−10 | 1.68E−12 |
| v28691 | 5.42E−08 | 1.8E−09 | 1.44E−09 | 1.89E−12 |
| v28692 | 4.64E−08 | 9.61E−10 | 7.71E−10 | 1.89E−12 |
| v28693 | 3.62E−08 | 7.77E−10 | 3.53E−10 | 3.34E−12 |
| v28694 | 3.47E−08 | 4.58E−10 | 4.55E−10 | 2.18E−12 |
| v20036 5G8 mouse-human | 2.66E−07 | 2.09E−08 | 1.69E−08 | 1.48E−12 |
| parental chimera | | | | |
| v28695 | DNB | — | ND | — |
| v28696 | 5.74E−07 | 9.76E−08 | NF | NF |
| v28697 | 7.35E−07 | 2.29E−08 | NF | NF |
| v28698 | 1.11E−06 | 1.25E−07 | 3.83E−08 | 1.35E−12 |
| v28699 | DNB | — | 4.71E−07 | 1.80E−12 |
| v28700 | 2.22E−07 | 3.95E−08 | 5.64E−08 | 1.64E−12 |
| v28701 | 6.10E−07 | 1.16E−08 | 3.00E−08 | 1.43E−12 |
| v28702 | 6.20E−07 | 6.35E−09 | 5.56E−08 | 1.46E−12 |
| v28703 | 8.11E−07 | 2.21E−08 | 3.58E−09 | 1.37E−12 |
| v28704 | 1.79E−07 | 2.31E−09 | 3.46E−09 | 1.35E−12 |
| v28705 | 1.95E−07 | 3.79E−09 | 3.32E−09 | 1.37E−12 |
| v28706 | 1.59E−07 | 5.77E−10 | 1.92E−09 | 2.05E−12 |
| v28707 | 7.32E−07 | 3.74E−08 | 5.16E−08 | 1.28E−12 |
| v28711 | 2.27E−07 | 3.51E−09 | 1.39E−07 | 6.05E−12 |
| v28712 | 2.29E−07 | 4.36E−09 | 3.56E−09 | 1.61E−12 |
| v28713 | 1.84E−07 | 6.43E−09 | 2.34E−09 | 1.41E−12 |

DNB = did not bind
ND = not tested
NF = no fit (for 4-parameter nonlinear regression model)

### Example 14

### Thermal Stability Assessment of Humanized Antibodies

In order to fully characterize humanized 1C8, 1G1 and 5G8 variants with affinity to human 4-1BB, the thermal stability of select antibody samples was assessed by differential scanning calorimetry (DSC) as described below.

The thermal stability of humanized 1C8, 1G1 and 5G8 antibody variants was measured using DSC as follows. 400 μL of purified samples primarily at concentrations of 0.4 mg/mL in PBS were used for DSC analysis with a VPT-Capillary DSC (GE Healthcare, Chicago, Ill.). At the start of each DSC run, 5 buffer blank injections were performed to stabilize the baseline, and a buffer injection was placed before each sample injection for referencing. Each sample was scanned from 20 to 100° C. at a 60° C./hr rate, with low feedback, 8 sec filter, 3 or 5 min pre-scan thermostat, and 70 psi nitrogen pressure. The resulting thermograms were referenced and analyzed using Origin 7 software to determine melting temperature (Tm) as an indicator of thermal stability.

### Results

The results are shown in Table 8 below.

TABLE 8
Thermal stability of humanized antibodies

| Variant Identifier | Fab Tm (° C.) |
|---|---|
| v20022 1C8 mouse-human | 88.1 |
| parental chimera | |
| v28726 | 92.0 |
| v28727 | 93.3 |
| v28730 | 94.0 |
| v20023 1G1 mouse-human | 73.2 |
| parental chimera | |
| v28683 | 84.0 |
| v28684 | 84.0 |
| v28685 | 83.4 |
| v28688 | 82.7 |
| v28689 | 81.8 |
| v28692 | 82.2 |
| v28693 | 81.1 |
| v20036 5G8 mouse-human | 82.6 |
| parental chimera | |
| v28696 | 90.8 |
| v28700 | 89.2 |
| v28704 | 89.9 |
| v28705 | 90.9 |
| v28711 | 90.5 |
| v28712 | 91.4 |

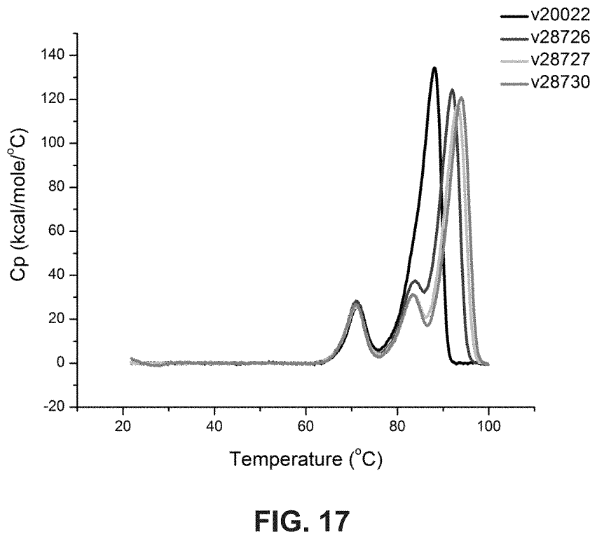

As can be seen in Table 8, determined Fab Tm values of select humanized 1C8 antibody variants are ˜-4-6° C. higher compared to the parental mouse chimera v20022. FIG. 17 shows the corresponding DSC thermograms of the 1C8 variants that were tested.

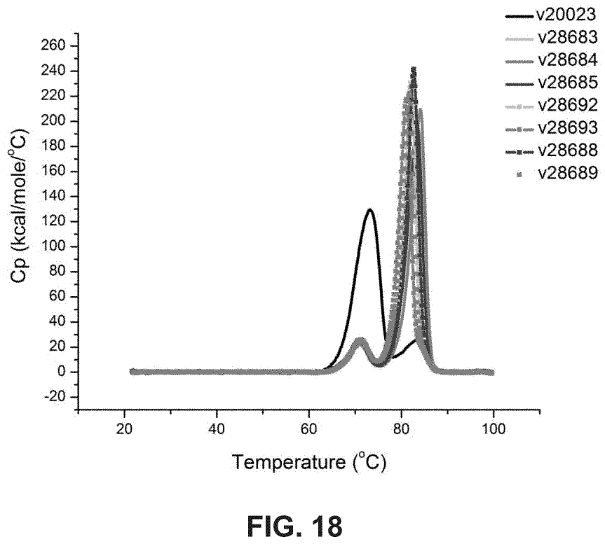

For humanized 1G1 antibody variants, as can be seen from Table 8, determined Fab Tm values of select variants are 9˜11° C. higher than the Tm of the parental mouse chimera v20023. FIG. 18 shows the corresponding DSC thermograms of the 1G1 variants that were tested.

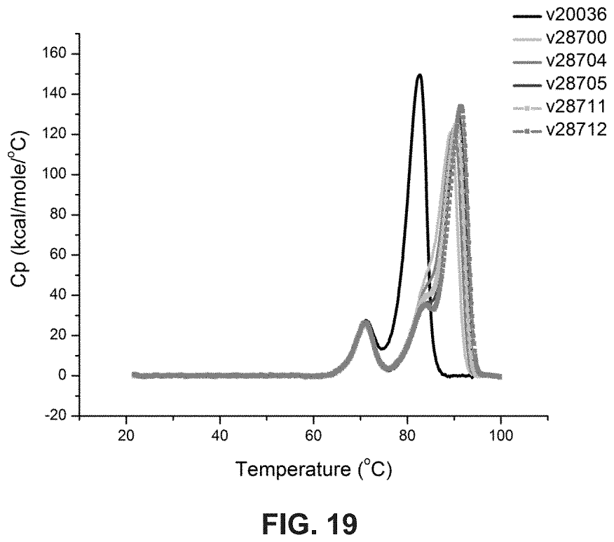

For humanized 5G8 antibody variants, as can be seen from Table 8, determined Fab Tm values of select variants are ˜7-9° C. higher than the Tm of the parental mouse chimera v20036. FIG. 19 shows the corresponding DSC thermograms of the 5G8 variants that were tested.

### Example 15

### Purity Assessment of Humanized 1C8, 1G1 and 5G8 Antibody Variants

The apparent purity of the humanized antibody variants prepared as described in Example 10 was assessed using mass spectrometry after non-denaturating deglycosylation. Samples of humanized variants were prepared and analyzed by LCMS as described in Example 1.

### Results

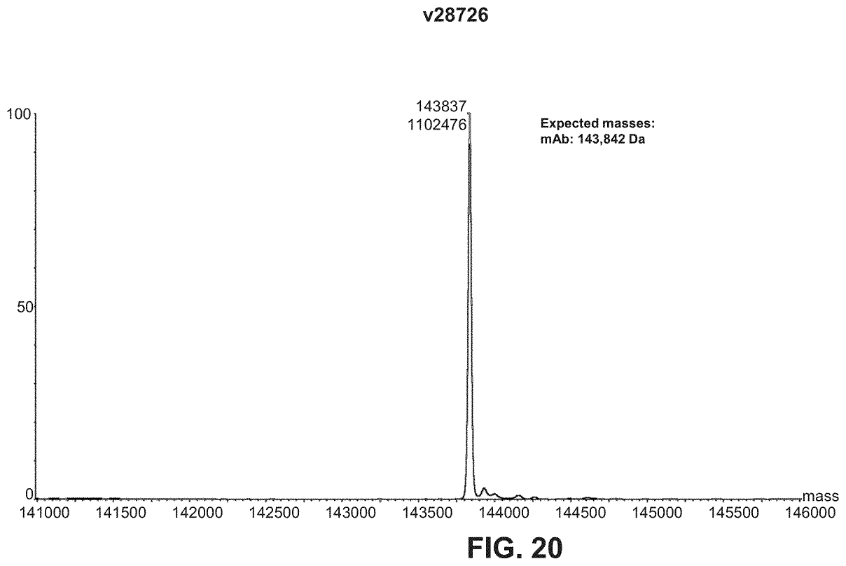

All humanized 1C8, 1G1 and 5G8 antibody variants were of 100% species purity. A representative LC-MS profile for one of the 1C8 humanized antibodies is shown in FIG. 20.

### Example 16

### Activation of 4-1BB by Humanized Antibodies

To determine if the humanized antibodies retained functionality post-humanization, they were tested in the 4-1BB NF-kB reporter gene assay according to the method described in Example 3.

### Results

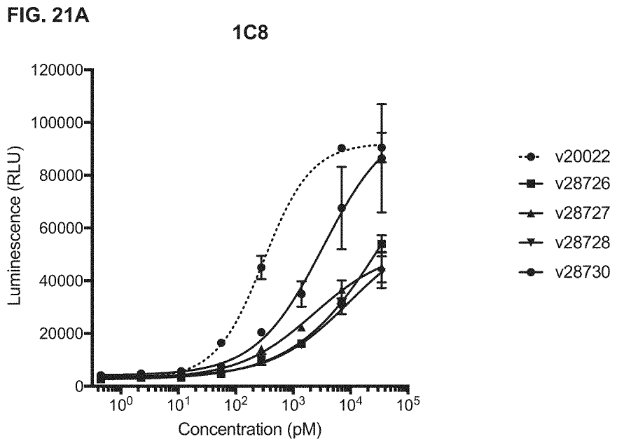

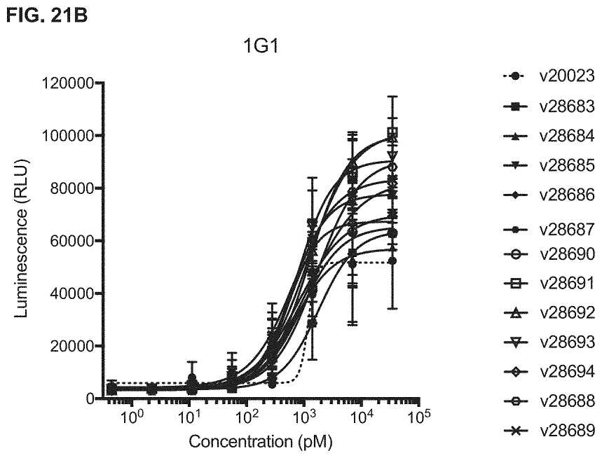

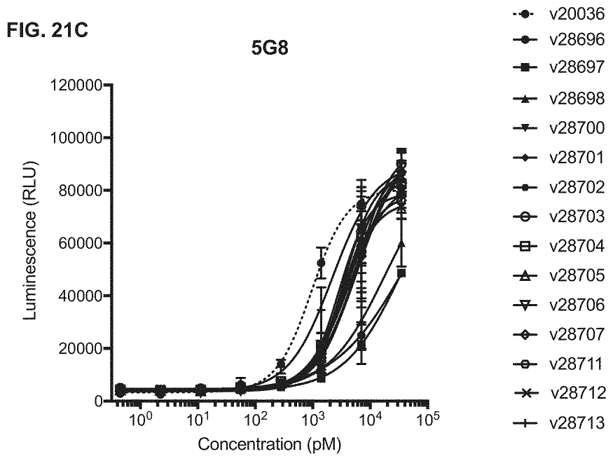

As seen in FIG. 21A, 1C8 showed a slight drop in potency compared to the parental v20022 antibody, as expected from the slight drop in binding seen in FIG. 16A. Similar to the flow cytometric binding in FIG. 16B, 1G1 retained functionality, with antibodies showing similar potency to the parental chimeric 4-1BB antibody v20023 (FIG. 21B). The humanized antibodies based on 5G8, similar to those based on 1C8, showed a slight decrease in potency compared to the parental antibody (FIG. 21C).

### Example 17

### Generation of Additional 4-1BB×TAA Antibody Constructs

The experiments described in Examples 1-3 identified a format in which 4-1BB×HER2 antibodies were able to cross-link 4-1BB and stimulate downstream 4-1BB signaling and the production of cytokine by T cells. To determine if this effect was specific to HER2 targeting or HER2-expressing tumours or if it can be also transferred to other tumour-associated antigens, 4-1BB×mesothelin (MSLN), 4-1BB×NaPi2b and 4-1BB×FRα antibodies were prepared.

### Design of 4-1BB×MSLN, 4-1BB×NaPi2b and 4-1BB×FRα Bispecific Antibodies

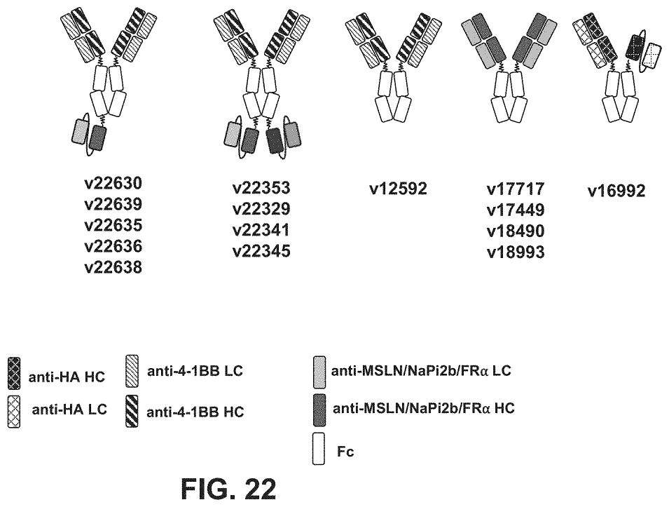

To allow testing of different tumour-associated antigens, bispecific antibody constructs were prepared in a similar format to the most active 4-1BB×HER2 bispecific constructs with two 4-1.BB Fab and one TAA scFv at the C-terminus of the Fc, as shown in FIG. 2B. Like the 4-1BB×HER2 bispecific antibody constructs described in Example 1, these bispecific antibody constructs comprised a human IgG1heterodimeric Fc having CH3 domain amino acid substitutions Het FcA and Het FcB, which drive association of the two component Fc polypeptides, Bispecific antibody constructs noted as “FcKO” included the following CH2 mutations designed to knock out or reduce FcγR binding: L234A, L235 A and D265S. Table 9 summarizes the antibody constructs that were prepared and FIG. 22 provides a representation of the formats of these antibody constructs. Control constructs 17717 (mirvetuximab), 17449 (farletuzumab), 18490 (RG7787), and 18993 (lifastuzumab) for each TAA paratope are also depicted in FIG. 22 and are described in Example 18.

TABLE 9
Description of 4-1BB × TAA antibodies

| | | Format | 4-1BB | TAA | Fc |
|---|---|---|---|---|---|
| Variant | TAA target | (4-1BB × TAA) | Paratope | Paratope | modifications |
| v22630 | Mesothelin | 2 × 1 | MOR7480.1 | Anetumab | FcKO |
| | | | | (VHVL) | |
| v22353 | Mesothelin | 2 × 2 | MOR7480.1 | Anetumab | FcKO |
| | | | | (VHVL) | |
| v22639 | Mesothelin | 2 × 1 | MOR7480.1 | RG7787 | FcKO |
| | | | | (VHVL) | |
| v22329 | Mesothelin | 2 × 2 | MOR7480.1 | RG7787 | FcKO |
| | | | | (VHVL) | |
| v22635 | NaPi2b | 2 × 1 | MOR7480.1 | MX-35 | FcKO |
| | | | | (VLVH) | |
| v22341 | NaPi2b | 2 × 2 | MOR7480.1 | MX-35 | FcKO |
| | | | | (VHVL) | |
| v22636 | NaPi2b | 2 × 1 | MOR7480.1 | Lifastuzumab | FcKO |
| | | | | (VHVL) | |
| v22345 | NaPi2b | 2 × 2 | MOR7480.1 | Lifastuzumab | FcKO |
| | | | | (VHVL) | |
| v22638 | FRα | 2 × 1 | MOR7480.1 | Mirvetuximab | FcKO |
| | | | | (VHVL) | |
| v12592 | 4-1BB | 2 × 0 | MOR7480.1 | None | FcWT |
| (control) | | | | | |
| v16992 | RSV | 0 × 0 | Palivizumab | None | FcKO |
| (control) | | | | | |

The sequences corresponding to the VH and VL of MOR7480.1 are provided in Table 15. The sequences of the scFvs used to construct the anti-TAA arm of the antibody constructs are provided in Table 16. Table X identifies the clones that make up each of the antibody constructs. The polypeptide sequences of each clone can be found in Table Y.

### Production of 4-1BB×TAA Antibodies

To allow the production of bispecific antibodies, constructs were made in a similar manner to Example 1.

### Production and Purification of Bispecific Antibodies

Antibodies were produced by transfecting CHO-2E7 cells, and purified using Protein A and prep-SEC, as described in Example 1. After purification, the antibodies were checked for purity and lack of aggregation using LC/MS and UPLC-SEC.

### Example 18

### Quantification of Surface TAA Protein on Tumor Cells

To determine what threshold of TAA expression is required on the tumour in order to stimulate 4-1BB signaling in T cells, the levels of mesothelia (MSLN), NaPi2b and FRα surface protein were measured in several tumour cell lines. This was achieved using quantitative flow cytometry using a set of beads with known levels of antibody bound as described below.

IGROV1, OVCAR3, H441, H661, H226. H1975 and A549 tumor cells were cultured in RPMI 10% FCS in 10 cm³ plates. These cell lines were chosen due to RNA data suggesting that they would be a representative set of Ovarian and bung cell lines expressing high, medium or low MSLN, NaPi2b and FRα. Cell dissociation buffer (Invitrogen) was added, and cells removed from the plate with mechanical means if necessary, using either a pipette or a cell scraper. Cells were left on ice with a pre-determined excess level of conjugated antibody, to ensure that the cells in the suspension are completely labelled. A series of beads with pre-determined levels of anti-human coating antibody were used as standards (816; Bangs Laboratories). Numbers of receptor per cell were calculated by comparing the level of AlexaFluor647 fluorescence on the tumour cells to a standard curve constructed using the calibration beads.

For conjugation with Alexa Fluor 647, antibodies were buffer exchanged into sodium bicarbonate buffer pH 8.4 using 40 kDa Zeba columns. An aliquot of each of the buffer exchanged material was then reacted with 10 eq. of NHS-Alexa Fluor 647 (Thermofisher A20006, 10 mM). Each reaction was allowed to proceed protected from light at room temperature for 90 minutes. Following incubation, each reaction was then purified. using a 40 kDa Zeba column, pre-equilibrated with PBS pH7.4. Conjugation was confirmed by SEC chromatography (Ex: 650 nm, Em: 665nm). SEC analysis also estimated the amount of unpurified NHS-Alexa Fluor 647.

| Target | Antibody Used | Anti-TAA paratope |
|---|---|---|
| FRα | v17717 | Mirvetuximab |
| FRα | v17449 | Farletuzumab |
| mesothelin (MSLN) | v18490 | RG7787 |
| NaPi2b | v18993 | Lifastuzumab |

### Results

Table 10 provides the results of surface TAA quantification and identifies tumour cell lines with high, medium and low expression of TAAs MSLN, FRα and NaPi2b.

TABLE 10
Surface TAA quantification on tumor cell lines

| | | MSLN | FRα | NaPi2b |
|---|---|---|---|---|
| | | (receptors/ | (receptors/ | (receptors/ |
| Cell line | Origin | cell) | cell) | cell) |
| IGROV1 | Ovarian Adenocarcinoma | 32917 | 1050311 | 995590 |
| OVCAR3 | Ovarian Adenocarcinoma | 288505 | 553495 | 1190046 |
| H226 | Lung Squamous | 1723332 | 307821 | 313349 |
| H441 | Lung Adenocarcinoma | 29239 | 186147 | 196188 |
| HCC827 | Lung Adenocarcinoma | 41161 | 177901 | 558926 |
| H661 | Lung Large Cell | 313615 | 176107 | 231227 |
| | Carcinoma | | | |
| H1573 | Lung Adenocarcinoma | 201484 | 136378 | 141558 |
| H1975 | Lung Adenocarcinoma | 156500 | 107420 | 103402 |
| H1563 | Lung Adenocarcinoma | 760672 | 75326 | 85979 |
| H1299 | Lung Carcinoma | 71333 | 39336 | 45395 |

### Example 19

### Ability of 4-1BB×TAA Bispecific Antibody Constructs to Stimulate 4-1BB Activity

To test the ability of bispecific 4-1BB×MSLN, 4-1BB×NaPi2b and 4-1BB×FRα antibodies to stimulate 4-1BB activity in the presence of a tumor cell, a co-culture reporter gene assay was employed.

### 4-1BB NF-kB-Lueiferase Reporter Assay

This experiment was undertaken similarly to the experiment in Example 3, except either H226, H661, H441, H1975, IGROV1, H1299 or A549 tumour cells were used. Briefly, NFκB-luc2P/4-1BB Jurkat cells were mixed with tumour cells in CD3-coated plates and left for 5 hours. The production of luciferase was then measured using Bio-Glo™ substrate, Data was analyzed using Prism 7 (GraphPad) and four-parameter variable slope nonlinear fit.

### Results

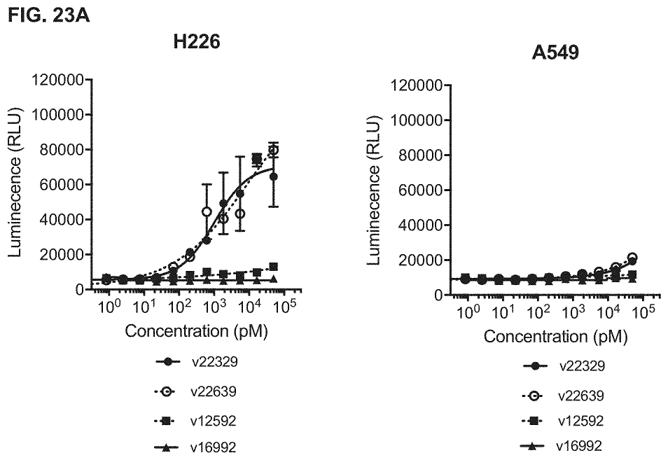

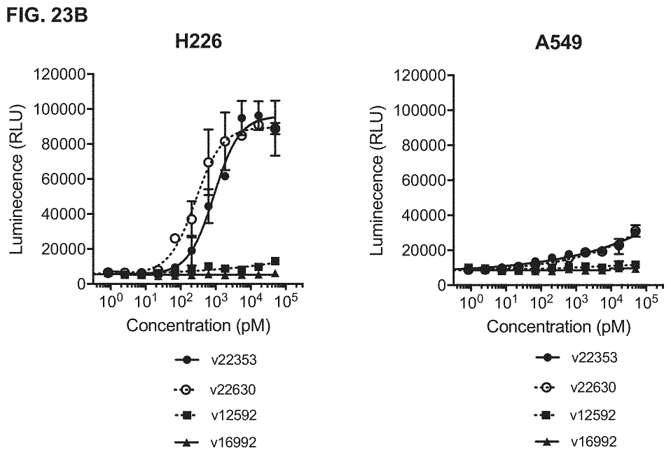

The results are shown in FIGS. 23A and 23B. 4-1BB×MSLN antibodies showed activity on H226 cells, but not A549 cells. v12592, which is a similar format antibody to the bispecific antibody construct but without the C-terminal anti-TAA say, does not show activity in this experiment on any of the cell lines, suggesting that crosslinking via the TAA may be necessary in order to function.

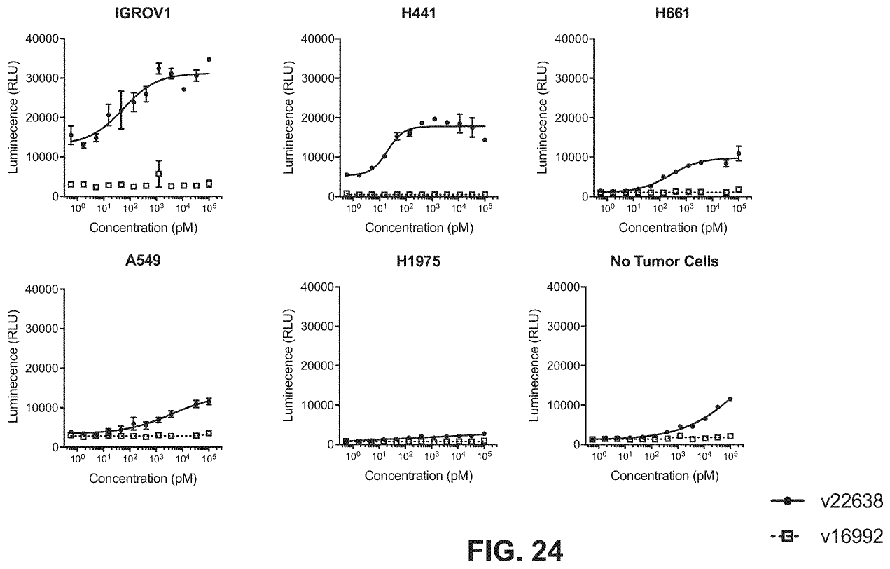

FIG. 24 shows the activity of the 4-1BB×FRα antibody v22638 on 4-1BB reporter cells in co-culture with a series of tumour lines representing a range of expression. When the 4-1BB reporter cells were cultured in presence of v22638 and tumour cells with greater than ˜150,000 FRα proteins per cell (IGROV1, H441, H661) an activation of the reporter genes was seen. In co-culture with tumour cells with lower levels of FRα, such as the H1299 cells, no activation of 4-1BB was seen. The ability of 4-1BB×FRα construct v22638 to stimulate 4-1BB activity appeared to be dependent on the level of FRα expression by tumour cells in this co-culture experiment. v22638 showed activity on FRα^{high} IGROV1 cells and FRα^{mid} H441 and H661 cells but did not shop: activity on FRα^{low} A549 or H1975 cells.

### Primary T Cell-Tumour Co-Culture Assay

Similar to Example 5, CD8+ cells were cultured with IGROV1, OVCAR3, H441, H661, H226, H1975 or A549 tumor cells and aAPC/CHO-K1 cells. After four days, supernatants were taken and IFNγ measured by HTRF. GraphPad Prism y7 was used for data analysis, using the non-linear four-parameter model.

### Results

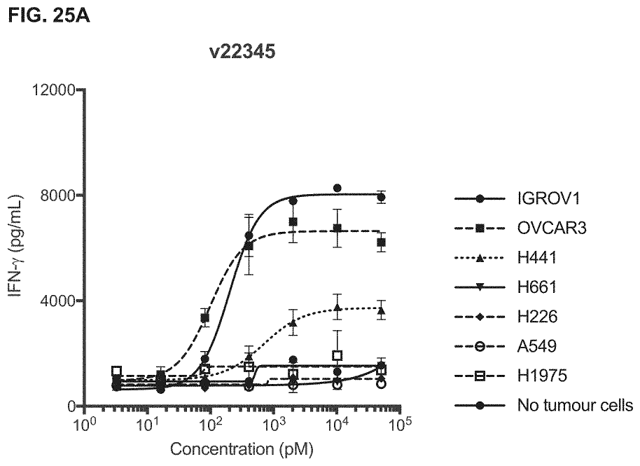

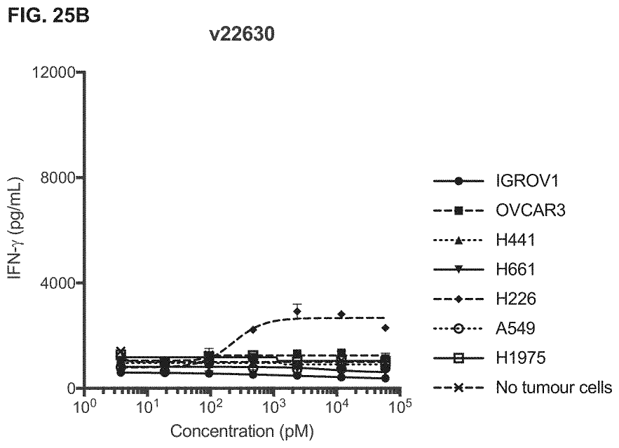

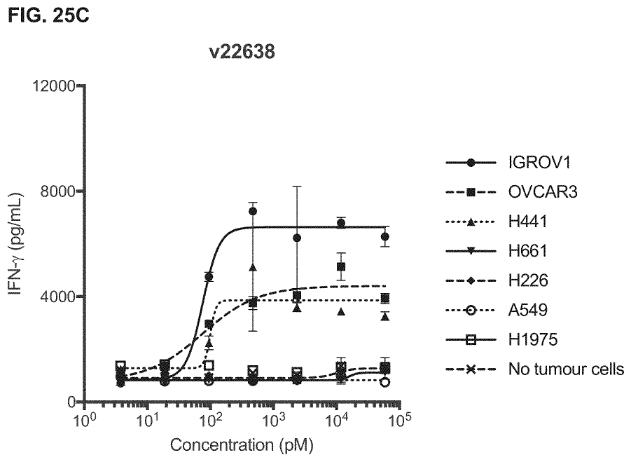

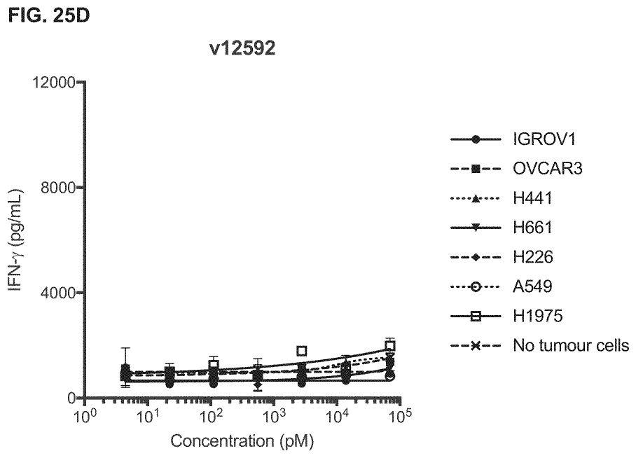

Similar to the results seen with the reporter gene assay, bispecific antibodies induced cytokine production by T cells when in co-culture with tumour cells expressing the cross-linking tumour antigen. The 4-1BB×MSLN antibody v22630 induced 1FNγ production by T cells when co-cultured with H226 cells which express high levels of MSLN, but not other tumour cells which express <300,000 MSLN molecules/cell (FIG. 25B). v22638, which is a bispecific antibody targeting 4-1BB and FRα shows activity on T cells in co-culture with IGROV1, OVCAR3 and H441 cells, suggesting a similar cut-off for expression of ˜200,000 FRα molecules/cell (FIG. 25C). The NaPi2b×4-1BB antibody construct v22345 was able to enhance IFNγ production by T cells co-cultured with NaPi2b^{high} IGROV1 or OVCAR3 cells and NaPi2b^{mid} H441 cells, but not NaPi2b^{mid-low} H661, F1226, A549 or H1975 cells. This suggests that a cut-off of ˜200,000-300,000 NaPi2b molecules/cell is required for function in vitro (FIG. 25A). No effect of v12592, the parental 4-1B.B antibody without a TAA cross-linking arm, was seen on the T cells in co-culture with any of the tumour cell lines, suggesting that cross-linking via the TAA arm was absolutely required for activity (FIG. 25D).

### Example 20

### Preparation of Additional 4-1BB×FRα Antibodies

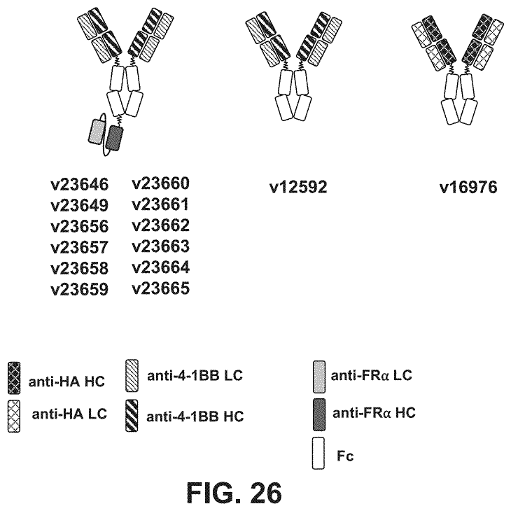

Additional 4-1BB×FRα antibody constructs (antibodies) were prepared according to the methods described in Example 1. Table 11 describes the compositions of these additional 4-1BB×FRα antibodies, while FIG. 26 provides a representation of the formats of exemplary antibodies. These 4-1BB×FRα antibody constructs were constructed using a subset of the mouse anti-4-1BB paratopes described in Example 7 that were shown to be agonistic to 4-1BB, and the anti-FRα paratopes mirvetuximab, rabbit paratope 1H06, and rabbit paratope 8K22. FRα paratopes 1H06 and 8K22 are novel rabbit anti-FRα paratopes generated as described in Example 23.

TABLE 11
Composition of 4-1BB × FRα antibodies

| | Format | 4-1BB | FRα |
|---|---|---|---|
| Variant | (4-1BB × FRα) | Paratope | Paratope |
| v23646 | 2 × 1 | 1C8 | 1H06 (VHVL) |
| v23649 | 2 × 1 | 5G8 | 1H06 (VHVL) |
| v23651 | 2 × 1 | 1C8 | Mirvetuximab |
| | | | (scFv) |
| v23656 | 2 × 1 | 1C8 | 8K22 (VLVH) |
| v23657 | 2 × 1 | 2E8 | 8K22 (VLVH) |
| v23658 | 2 × 1 | 4E6 | 8K22 (VLVH) |
| v23659 | 2 × 1 | 5G8 | 8K22 (VLVH) |
| v23660 | 2 × 1 | 6B3 | 8K22 (VLVH) |
| v23661 | 2 × 1 | 1C8 | 1H06 (VLVH) |
| v23662 | 2 × 1 | 2E8 | 1H06 (VLVH) |
| v23663 | 2 × 1 | 4E6 | 1H06 (VLVH) |
| v23664 | 2 × 1 | 5G8 | 1H06 (VLVH) |
| v23665 | 2 × 1 | 6B3 | 1H06 (VLVH) |
| v20022 | 2 × 0 | 1C8 | None |
| v20036 | 2 × 0 | 5G8 | None |
| v12592 (control | 2 × 0 | MOR7480.1 | None |
| monospecific anti- | | | |
| 4-1BB antibody) | | | |
| v16976 (negative | 0 × 0 | None | None |
| control, CR8071 | | | |
| antibody with Het | | | |
| FcA, FcB, and FcKO | | | |
| Fc modifications) | | | |
| v17721 (control | 0 × 1 | None | Mirvetuximab |
| one-armed anti- | | | (scFv) |
| FRα antibody) | | | |

Table X identifies the clones that make up each of the antibody constructs. The polypeptide sequences of each clone can be found in Table Y.

The expressed and purified antibodies were then tested as described in Examples 21 and 22.

### Example 21

### Characterization of 4-1BB×FRα Antibody Constructs Binding to 4-BB and FRα

To test the ability of the 4-1BB×FRα antibody constructs produced in Example 20 to bind 4-1BB, the affinity of these constructs for human 4-1BB was measured by SPR and by flow cytometry.

### SPR

Variants purified by SEC were assessed for binding to human 4-1BB. The antigen-binding affinity was determined by SPR according to the method described in Example 2. A summary of the SPR binding data is provided in Table 12.

TABLE 12
Binding data for 4-1BB × FRα bispecific antibodies

| Variant | KD (M) |
|---|---|
| v12592 | 1.87 × 10⁻⁸ |
| v23656 | 4.8 × 10⁻⁷ |
| v23657 | 1.75 × 10⁻⁷ |
| v23658 | 4.99 × 10⁻⁷ |
| v23659 | 2.08 × 10⁻⁷ |
| v23660 | 3.64 × 10⁻⁷ |
| v23661 | 5.35 × 10⁻⁷ |
| v23662 | 1.55 × 10⁻⁷ |
| v23663 | 5.02 × 10⁻⁷ |
| v23664 | 2.2 × 10⁻⁷ |
| v23665 | 4.64 × 10⁻⁷ |

All of the 4-1BB×FRα antibodies tested showed binding to 4-1BB, and had a KD representing affinity that was between approximately 20-100 fold lower than the control anti-4-1BB antibody MOR7480.1 (v12592) as measured by SPR.

### Binding of 4-1BB×FRα Antibody Constructs to 4-1BB-Expressing Jurkat T Cells by Flow Cytometry

To examine binding of these antibodies to native cell surface-expressed 4-1BB, a flow cytometry binding assay was carried out.

Jurkat T cells engineered to stably express human 4-1BB were used to measure binding of antibodies to human 4-1BB. Antibodies were diluted in 50 μl FB (PBS 2% FCS) 1:3 from stock in wells of a 96 V-well plate, and cells added on top. The cells were then left on ice for 30 minutes for the antibodies to bind. The cells were then washed twice in FB, and incubated in 50 μl FB containing 2 μg/ml goat anti-human Alexa647 antibody (Jackson Immunoresearch). The cells were then left on ice for a further 20 minutes, washed twice in FB, resuspended in 100 μl FB and analysed on a BD Fortessa™ X20. The subsequent data files were analysed using FlowJo™ and Prism™ 7 (GraphPad) using a four-parameter non-linear regression.

### Results

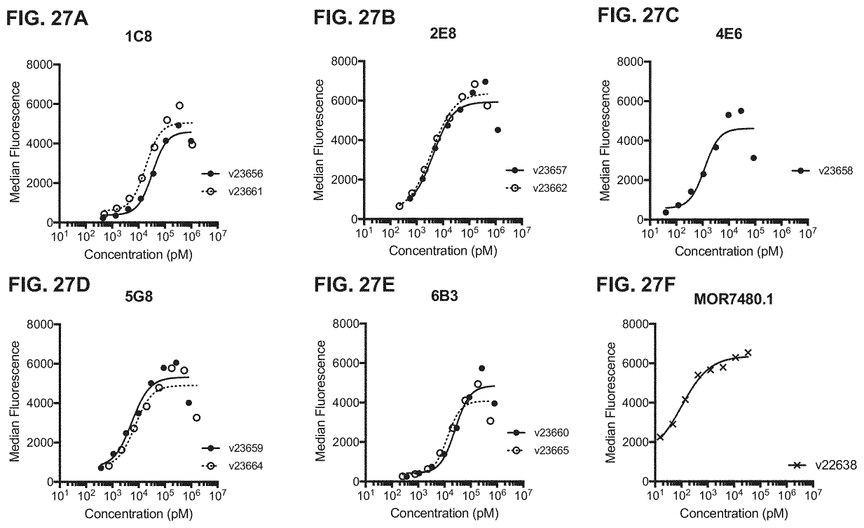

All variants except v23663 showed binding. Similar to the SPR results, the antibodies tested in this experiment showed lower affinity compared to v12592 (FIG. 27A to 27F). FIG. 27F shows the results for the control variant 22638, a 4-1BB×FRα bispecific antibody with MOR7480.1 (4.-1BB) and mirvetuximab (FRα) paratopes.

### Binding of 4-1BB×FRα Antibody Constructs to FRα Expressed on 293E Cells by Flow Cytometry

To examine binding of antibodies to FRα expressed on the cell surface, 293E cells were transiently transfected with full-length FRα (SEQ ID NO:80). Antibodies were diluted in 50 μl FB (PBS 2% FCS) 1:3 from stock in wells of a 96 V-well plate, and cells added on top. The cells were then left on ice for 30 minutes for the antibodies to bind. The cells were then washed twice in FB, and incubated in 50 μl FB containing 2 μg/ml goat anti-human Alexa647 antibody (Jackson Immunoresearch). The cells were then left on ice for a further 20 minutes, washed twice in FB, resuspended in 100 μl FB and analyzed on a BD Fortessa™ X20. The subsequent data files were analyzed using FlowJo™ and Prism 7 (GraphPad).

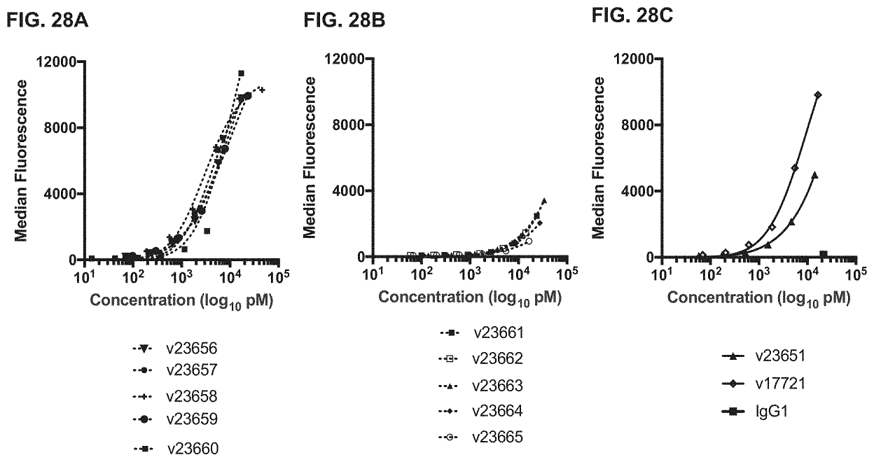

The results are shown in FIG. 28 and demonstrate that all antibodies showed binding to FRα. Sample containing the 8K22 scFv (FIG. 28A) showed higher binding than the 1H06 scFv (FIG. 28B), suggesting that it is of higher affinity as an scFv. Antibodies containing a mirvetuximab scFv (FIG. 28C) showed intermediate binding between 8K22 and 1H06, suggesting that its affinity is between the two.

### Example 22

### Activation of T Cells by 4-1BB×FRα Bispecific Antibodies

After confirmation of binding of 4-1BB×FRα antibodies to both 4-1BB and FRα, the bispecific antibodies were examined in a primary T cell activity assay. The experiment looked at the ability of 4-1BB to stimulate the production of IFNγ by T cells in the culture. Co-culture of T cells with tumour cells allowed the investigation of crosslinking of the 4-1BB antibodies by TAA on tumour cells. IGROV1. cells were chosen due to their high expression of FRα, and A549 for low expression of FRα.

The method used was similar to that used in Example 5. Bispecific antibodies, CD8+ T cells and either IGROV1 or A549 tumour cells were cultured together with aAPC/CHO-K1cells. After four days, supernatants were taken and IFNγ measured by HTRF.

### Results

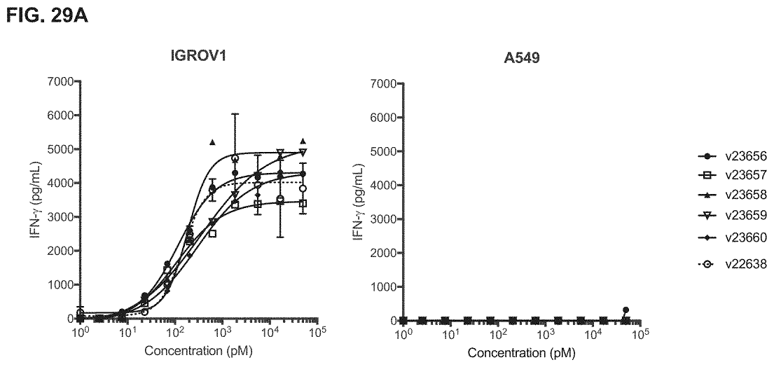

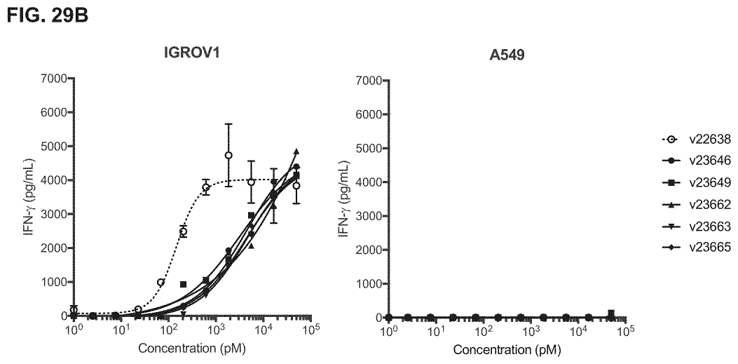

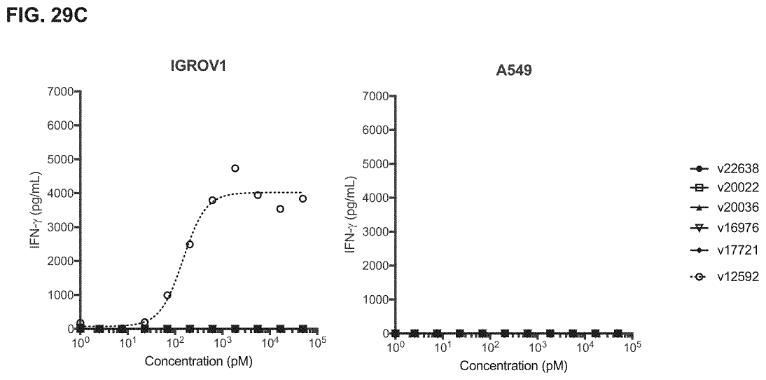

All 4-1BB×FRα antibodies stimulated IFNγ production by T cells when in co-culture with FRα^{high} IGROV1 cells (FIG. 29A and 29 B). In culture with FRα^{low} A549 cells, there was no effect of the 4-1BB×FRα antibodies seen on the T cells, suggesting that this cell line may not express FRα at a level sufficient for the 4-1BB×FRα antibodies to stimulate IFNγ production by T cells. In the absence of a tumour-targeting arm, 4-1BB monospecific antibodies v12592, v20022 and v20036 could not stimulate cytokine production when in culture with IGROV1 or A549 cells (FIG. 29C). v22368 acted as a positive control and comparator.

It was also found that 4-1BB antibody affinity did not affect the response of the T cells in this experiment. The difference in activity between the antibodies may be due to the difference in binding between the 1H06 and 8K22 scFvs (8K22 demonstrated greater binding to FRα by flow cytometry than did 1H06, and also showed greater activity in stimulating IFNγ production).

### Example 23

### Generation of Rabbit Antibodies that Bind Human FRα

Antibodies to Folate receptor alpha (FRα) were raised in rabbits immunized with soluble HIS tagged human folate receptor 1 antigen (FRα-HIS, AcroBiosystems Cat #FO1-F182E2). The 8K22. and 1H06 paratopes described in Example 20 were identified by the method described here.

Briefly, New Zealand white rabbits were given a primary injection followed by 4 additional boosts of the FRα-HIS antigen mixed with adjuvant. Each of the boosts were separated by 14 days. Anti human FRα antibodies titers were determined by FACs using transiently expressing human FRα CHO cells to choose which animal to harvest for B cells.

### Recovery of B Cells and Discovery of Anti-Human FRα Antibodies by SLAM:

Immunized rabbits with desired titers about 100,000 were sacrificed, and the spleens harvested. The lymphoid cells were dissociated by grinding in FACs buffer (PBS 2% FBS) to release the cells from the tissues. The cells were pelletal and then suspended for 1 min in 5 ml of BD Pharm Lyse to lyse red blood cells. Equal volume of FACs buffer was added to neutralize the Pharm Lyse and the resultant lymphocyte sample was pelleted and suspended in FACs buffer.

The lymphocyte suspension was then stained with anti-rabbit IgG Alexa-Fluor 647 to identify IgG+ B cells. After 30 min of staining, IgG+ B cells were sorted on a FACSAria (BD Biosciences) and counted. Using the Selected Lymphocyte Antibody Method (SLAM) (Proc Natl Acad Sci USA. 1996 Jul. 23; 93(15): 7843-7848. John Babcook et al), B cells were plated at different densities ranging from single cell up to 50 cells in a 384 well plate, expanded in culture for 7 days and the supernatants harvested to detect for anti-human FRα antibodies. The 384 well ;gates were frozen down in a −80 C freezer.

Supernatants were screened for human FRα specific monoclonal antibodies by ELISA. 384 well ELISA plates were coated with 25 μL/well of human FRα-HIS (2 μg/mL) in PBS, then incubated at 4° C. overnight. After incubation, the plates were washed with water 2 times. 90 μL/well Blocking Buffer (2% skim milk, PBS) were added and the plates incubated at room temperature for 1 hour. After incubation, the plates were washed and 12.5 μL/well of antibody containing supernatants+12.5 μl Blocking Buffer, and positive and negative controls were added and the plates incubated at room temperature for 2 hours.

After incubation, the plates were washed, 25 μl of 0.4 ug/ml goat anti-rabbit IgG Fc-HRP detection antibody was added to each well and the plates were incubated at room temperature for 1 hour. After the incubation, the plates were washed and 25 μl of TMB were added and the plates allowed to develop for about 10 minutes (until negative control wells barely started to show color). Then 25 μl stop solution (1N HCL) were added to each well and the plates read on an ELISA plate reader at wavelength 450 nm.

### Sequencing of Anti-Human FRα Monoclonal Antibodies:

Wells containing antibodies of desired characteristics were treated with RNA lysis buffer (Qiagen RNeasy) for molecular rescue of antibody heavy and light chains. Initial PCR of heavy and light chain antibody-coding sequences was performed using primers and methods modified from Babcook et al. (Proc Natl Acad Sci USA 1996 Jul. 23; 93(15): 7843) and von Boehmer et al. (Nat Protoc. 2016 October; 11(10): 1908), with cDNA as the nucleic acid template. PCR products were cloned into the pCRTOPO4 vector using the Zero Blunt™ TOPO™ PCR. Cloning kit (Thermofisher Scientific) and transformed into E. cloni™ cells (Lucigen). Antibiotic-resistant clones were sequenced and analyzed for unique antibody-coding sequences.

A nested PCR reaction was then performed on these unique sequences using V-segment family and J-segment family-specific primers. The resulting amplicons were then cloned into pTT5-based expression plasmids (National Research Council of Canada). Unique heavy chain sequences and light chain sequences emerging from a single well sample were co-expressed in HEK293-6E cells (National Research Council of Canada) in all possible combinations to determine the correct heavy and light chain pairing. Antibodies produced were assayed for binding to antigen that was transiently expressed on HEK293 cells.

### Example 24

### Humanization of Rabbit 8K22 VH and VL Sequences

A rabbit anti-human folate receptor alpha (anti-hFRα) antibody, 8K22, which was generated as described in Example 23, was humanized as described below.

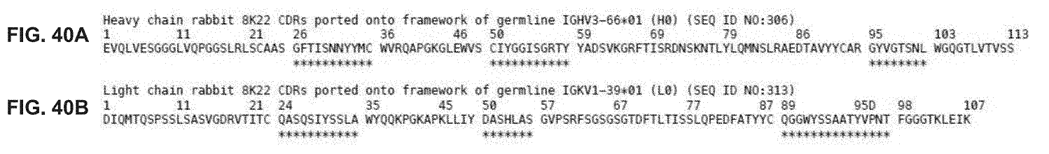

Sequence alignment of rabbit 8K22 VH and VL sequences to respective human germline sequences identified IGHV3-66*01 and IGKVI-39*01 as the closest, as well as frequent, human germline sequences. CDRs according to the AbM definition (<http://www.bioinf.org.uk/abs/#cdrdef>) were ported onto the framework of these selected human germline sequences as shown in FIG. 40. Back mutations to rabbit residues in the resultant sequences at positions judged likely to be important for the retention of binding affinity to antigen, hFRα, were included creating several humanized sequences in which generated sequences for the most part built on the previous sequence, and where the first humanized sequence (H1 and L1, Table 19) contained the minimal number of back mutations. None of the variants modified the CDRs of the 8K22 antibody as defined by the AbM method.

This process provided five variable heavy chain humanized sequences and five variable light chain humanized sequences. Full heavy chain sequences containing humanized heavy chain variable domain (VH) and hIgG1 heavy chain constant domains (CH1, hinge, CH2, CH3), and full light chain sequence containing humanized light chain variable domain (VL) and human kappa light chain constant domain (kappa CL) were assembled. Monoclonal antibody (mAb) variants were then assembled such that each of the humanized heavy chains was paired with each of the humanized light chains, providing a total of twenty-five humanized variants to be evaluated experimentally (Table 19).

### Example 25

### Humanized 8K22 Antibody Production

The humanized 8K22 antibodies described in Example 24 and Table 19 were prepared as follows.

Each of the humanized 8K22 constructs, as well as the parental 8K22 construct, were in the naturally occurring or FSA format, containing two identical full-length heavy chains and two identical kappa light chains. The amino acid sequences of each of the antibody variable heavy chains and variable light chains are provided in Table 20. Each of the humanized VH domain sequences (SEQ ID NOs: 307, 308, 309, 310 and 312) was appended to the human CH1-hinge-CH2-CH3 domain sequence of IGHG1*01 (SEQ ID NO: 318) to provide five humanized 8K22 full heavy chain sequences. Each of the humanized VL domain sequences (SEQ ID NOs: 313, 314, 315, 316 and 317) was appended to tie human kappa CL sequence of IGKC*01 (SEQ ID NO:67) to provide five humanized 8K22 light chain sequences. In a similar manner, 8K22 rabbit-human parental antibody chimera heavy and light chain sequences were assembled, with the difference that variable domain sequences were rabbit (SEQ ID NOs:298 (VH) and 299 (VL)) and constant domain sequences were human (SEQ ID NOs: 318 (CH1-hinge-CH2-CH3 chain) and 67 (CL sequence of IGKC*01)). These sequences were reverse translated to DNA, codon optimized for mammalian expression and gene synthesized. The humanized VH and VL sequences are provided in Table 20.

Heavy chain vector inserts comprising a signal peptide (artificially designed sequence: MRPTWAWWLFLVLLLALWAPARG (SEQ ID NO:1) (Barash er al., (2.002), Biochem and Biophys Res. Comm., 294:835-842)) and the heavy chain clone terminating at G446 (EU numbering) of CH3 were ligated into a pTT5 vector to produce heavy chain expression vectors. Light chain vector inserts comprising the same signal peptide were ligated into a pTT5 vector to produce light chain expression vectors. The resulting heavy and light chain expression vectors were sequenced to confirm correct reading frame and sequence of the coding DNA.

The heavy and light chains of the antibody variants were expressed in 400 ml cultures of CHO-3E7 cells. Briefly, CHO-3E7 cells, at a density of 1,7-2×10⁶ cells/ml, viability >95%, were cultured at 37° C. in FreeStyle™ F17 medium (ThermoFisher, Watham, Mass.) supplemented with 4 mM glutamine (GE Life Sciences, Marlborogh, Mass.) and 0.1% Pluronic® F-68 (Gibco, Life Technologies), A total volume of 400 ml was transfected with a total of 400 ug DNA (200 ug. of antibody DNA and 200 ug of GFP/AKT/stuffer DNA) using PEI-max (Polyscience, Philadelphia, Pa.) at a DNA:PEI ratio of 1:4 (W/W). Twenty-four hours after the addition of the DNA-PEI mixture, 0.5 mM valproic acid (final concentration)+1% w/v Tryptone (final concentration)+1× antibiotic/antimycotics (GE Life Sciences, Marlborogh, Mass.) were added to the cells, which were then transferred to 32° C. and incubated for 9 days prior to harvesting. The parental 8K22 rabbit-human antibody chimera was expressed in a similar manner in a 1 L culture.

Clarified supernatant samples were incubated in batch with mAb Select SuRe resin (GE Healthcare, Chicago, Ill.) cleaned-in-place (CIP'd) with NaOH and equilibrated in Dulbecco's PBS (DPBS). Resin was poured into CIP'd columns, the columns were washed with DPBS and protein eluted with 100 mM sodium citrate buffer pH 3,0. The eluted fractions were pH adjusted by adding 10% (v/v) 1M HEPES pH 8 to yield a final pH of 6-7. Samples were buffer exchanged into PBS and aseptically filtered. Protein was quantitated based on absorbance at 280 nm (A280 nm) (in instances where precipitation was present upon sample neutralization, these samples were centrifuged briefly prior to A280 nm measurements). Endotoxin levels were determined using the Endosafe® Portable system (Charles River, Wilmington, Mass.). Samples having endotoxin above 0.2 EU/mg underwent endotoxin removal with the NoEndo™ Spin columns (Viva Products Inc., Littleton, Mass.). Parental 8K22 rabbit-human antibody chimera variant was further purified by preparatory SEC chromatography (Superdex 200 26/60) in DPBS mobile phase following protein-A purification.

Following purification, purity of samples was assessed by non-reducing and reducing High Throughput Protein Express assay using Caliper LabChip® GXII (Perkin Elmer, Waltham, Mass.). Procedures were carried out according to HT Protein Express LabChip® User Guide version 2 with the following modifications. Antibody samples, at either 2 μl or 5 μl (concentration range 5-2000 ng/μl), were added to separate wells in 96 well plates (BioRad, Hercules, Calif. ) along with 7 μl of HI Protein Express Sample Buffer (Perkin Elmer #4760328). Antibody samples were then denatured at 70° C. for 15 mins. The LabChip® instrument was operated using the HT Protein Express Chip (Perkin Elmer, Waltham, Mass.) and the Ab-200 assay setting.

### Results

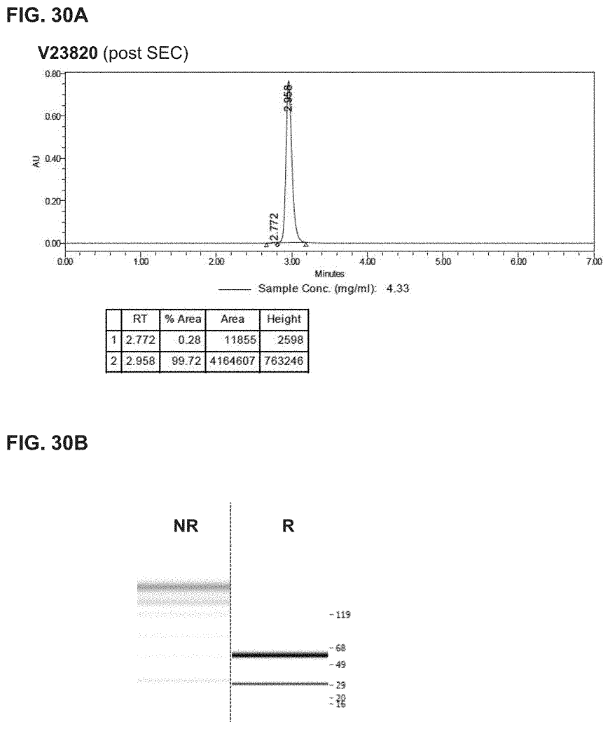

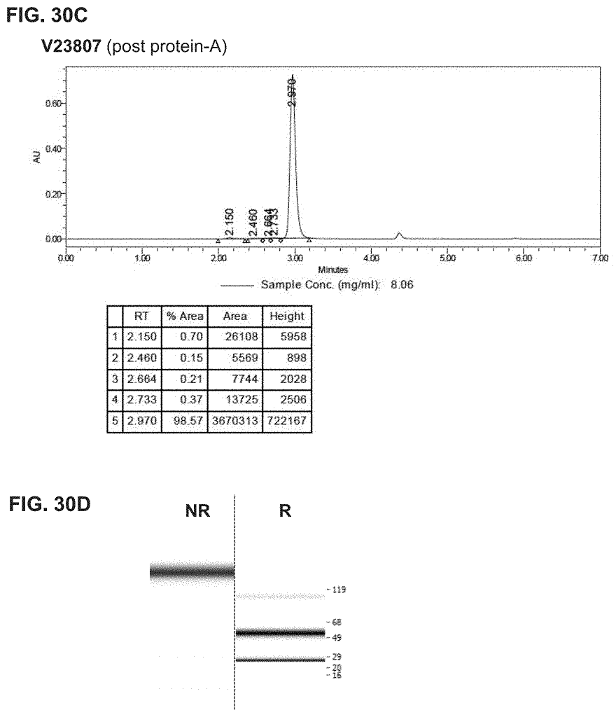

Yield post protein-A purification across the twenty-five humanized 8K22 antibody variants ranged from ˜10-30 mg (or 25-75 mg/L ). FIG. 30B and 30D shows the Caliper result for the parental chimeric antibody v23820 and a representative humanized variant, v23807. As shown in FIG. 30D, on a representative humanized antibody sample, non-reducing (NR) and reducing (R) Caliper reflected a single species corresponding to full-size antibody and intact heavy and light chains, which was the case with all humanized variants. Small levels of precipitation were observed upon sample neutralization following protein-A elution, for the following variants: 23804, 2805, 23807, 23808, 23814, 23816, 23817, 23818, including the parental chimera, v23820. Comparison with protein samples of similar titers that did not result in any precipitation, suggested that levels of precipitation observed were relatively negligible, since the resulting yields for these two types of samples were comparable. Some of the humanized 8K22 antibody samples required endotoxin removal following protein-A purification. Endotoxin removal was carried out for two out of twenty-five humanized 8K22 antibody samples and resulted in successful reduction of endotoxin levels to the necessary specifications.

### Example 26

### Quality Assessment of Purified Humanized 8K22 Antibodies

Samples of humanized 8K22 antibody variants were subjected to UPLC-SEC in order to assess species homogeneity following protein-A purification or following preparatory SEC purification in the case of parental chimera antibody, v23820.

UPLC-SEC was performed using a Waters Acquity BEH200 SEC column (2.5 mL, 4.6×150 mm, stainless steel, 1.7 μm particles) (Waters LTD, Mississauga, ON) set to 30° C. and mounted on a Waters Acquity UPLC H-Class Bio system with a photodiode array (PDA) detector. Run times consisted of 7 min and a total volume per injection of 2.8 mL with a running buffer of DPBS with 0.02% Tween 20 pH 7.4 at 0.4 mL/'min, Elution was monitored by UV absorbance in the range 210-500 nm, and chromatograms were extracted at 280 nm. Peak integration was performed using Waters Empower 3 software.

### Results

As shown in FIGS. 30A (for the parental chimera v23820) and 30C (for a representative humanized antibody), UPLC-SEC profile for the representative humanized antibody sample reflected high species homogeneity, comparable to the purified parental chimera antibody sample. The parental chimera contained higher molecular weight species following protein-A purification (not shown), which were removed by preparatory SEC. The rest of the humanized 8K22 antibody samples had similar profiles to those of the representative humanized antibody sample.

### Example 27

### Affinity Assessment of Humanized 8K22 Antibodies for hFRα

To determine whether the humanization process affected the affinity of the humanized variants for their target, the ability of the humanized 8K22 antibody variants to bind the hFRα antigen was assessed by Bio-layer interferometry (BLI).

Supernatant material post-harvest was screened for binding to the hFRα, followed by the binding assay repeat on the purified antibody samples for selected variants.

Antigen binding was assessed using the Octet RED96 system by cycling through the following steps: loading of antibodies (0.9 μg/mL) onto AHC biosensors over 200 s; stabilization of baseline for 60 s; association to recombinant His-tagged human FRα (Acrobiosystem) at multiple relevant concentrations spanning the expected KD for 500 s; dissociation was recorded for 1200 s; and regeneration was performed by cycling 3 times between 10 mM glycine pH 1.5 (15 s) and the assay buffer (15 s) before proceeding to the next antibody. The assay buffer used was KB buffer (kinetics buffer, composed of PBS pH 7.4, 0.1% BSA, 0.02% Tween 20, 0.05% sodium azide) supplemented with 0.06% Tween 20. The experiment was conducted at 25° C. with a shake speed of 1000 rpm.

Data analysis was performed using the Data analysis software 9.0′ (ForteBio). The reference-subtracted binding curves were globally fitted to the 1:1 interaction model to generate the binding kinetic parameters kₒₙ, k_{off}, and the dissociation constant KD.

### Results

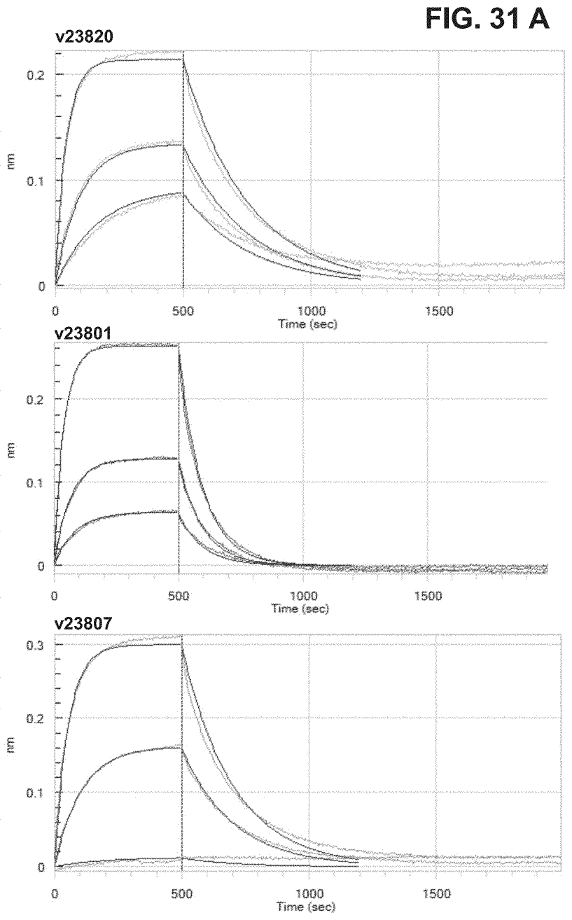

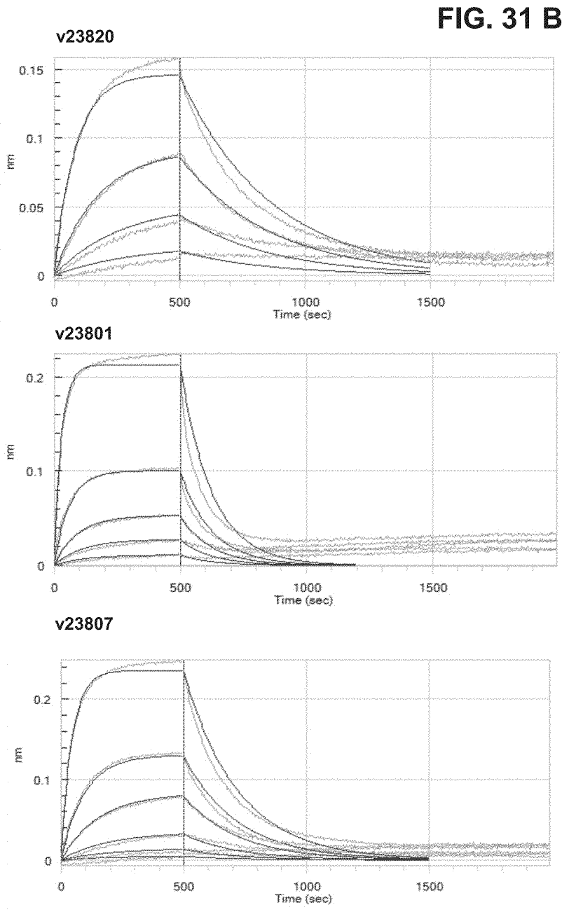

The results are shown in Table 21 and FIG. 31. FIG. 31A shows the BLI sensorgrams for the parental chimeric antibody v23820, and two representative humanized antibodies, v23801 and v23807 using supernatants. FIG. 31B shows the BLI sensorgrams for the parental chimeric antibody v23820, and two representative humanized antibodies, v23801 and v23807 using purified antibodies. Screening of antibody supernatants for binding to hFRα distinguished a top group (Group A) of humanized 8K22 antibody variants (variants 23798, 23804, 23806, 23807, 23809, 23814, 23816 and 23817) with minor reduction in affinity, within 2-fold, compared to that of the parental chimera antibody. Obtained KD values ranged from ˜14 nM to 9.3nM, with KD of the parental chimera antibody (variant 23820) determined to be 5.9 nM. The majority of the humanized 8K2.2 antibody variants were characterized by greater than 2-fold and up to 4-fold reduced affinity compared to that of the parental chimera mAb; these are referred to as Group B variants. Variants 23795, 23800, 23810, 23803 and 23813 exhibited a further decrease in affinity. ˜5-6 fold compared to the parental chimera mAb; these variants are referred to as Group C variants. Differences in determined KD values between humanized 8K22 antibody variants primarily stemmed from differences in K_{off} values.

BLI binding assay on purified antibody samples was consequently performed for the variants that exhibited up to ˜4-fold reduction in affinity as determined in the assay performed on the supernatant material. Absolute KD values obtained in this assay, performed on purified material, were systematically lower than those obtained in the assay performed on the supernatant material, due to higher Kₒₙ values (K_{off} values were largely comparable to those obtained in the assay performed on the supernatant material), however the relative ranking of 8K22 humanized variants was very similar. Differences in the placement within Groups A, B and C were observed for variants 23809 and 23816 (from Group A to Group B), as well as for the variants 23794 and 23818 (from Group B to Group A).

Variants 23804, 23806, 23807, 23814 and 23817 emerged as the top tier performing variants, with respect to the retention of the affinity to liFRα within 2-fold upon humanization, as determined by both binding assays, performed on supernatant and purified sample material. These variants have L3 or L5 humanized light chains in common, which differ fr©m the rest of the three humanized light chains by the presence of the two amino acid back substitutions to rabbit residues in the FR loop. Data obtained in these binding assays suggests that these two particular amino acid residues are important for retaining parental chimera-like antigen binding affinity in humanized variants. Secondary determinants of the top tier antibody variants that emerged are the presence of H1 or H4 humanized heavy chains.

### Example 28

### Thermal Stability Assessment of Humanized 8K22 Antibodies

The thermal stability of humanized 8K22 antibody variants was assessed by differential scanning calorimetry (DSC) as described below.

400 μL of purified samples primarily at concentrations of 0.4 mg/mL in PBS were used for DSC analysis with a VP-Capillary DSC (GE Healthcare, Chicago, Ill.). At the start of each DSC run, 5 buffer blank injections were performed to stabilize the baseline, and a buffer injection was placed before each sample injection for referencing. Each sample was scanned from 20° C. to 100° C. at a 60° C./hr rate, with low feedback, 8 sec filter, 3 min pre-scan thermostat, and 70 psi nitrogen pressure. The resulting thermograms were referenced and analyzed using Origin 7 software (OriginLab Corporation, Northampton, Mass.) to determine melting temperature (Tm) as an indicator of thermal stability.

### Results

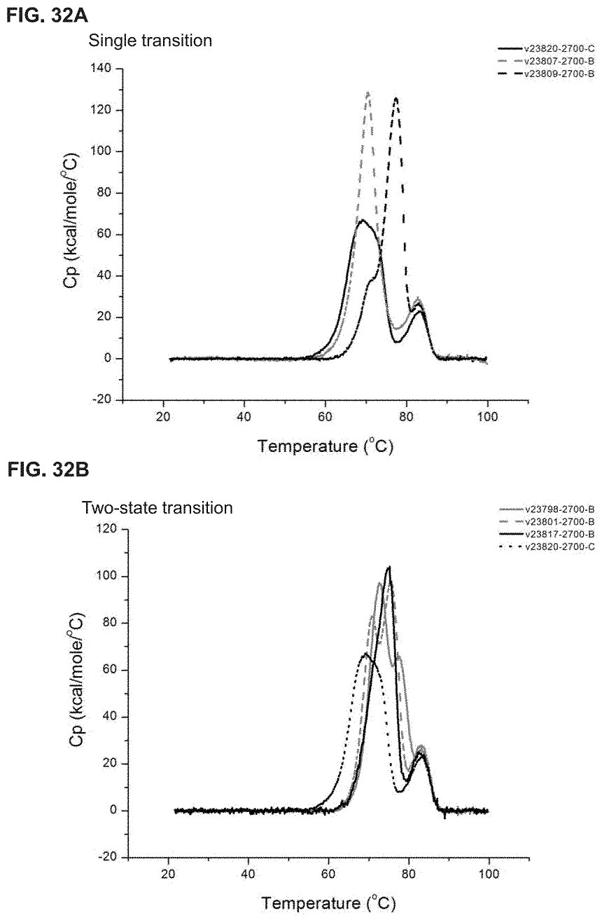

Fab Tm values were determined for the humanized 8K22 antibody variants that exhibited lower than ˜5-6-fold reduction in antigen affinity compared to the parental chimera antibody. Determined Fab Tm values for the characterized humanized variants, were comparable or up to 10° C. higher than that of the parental chimera antibody, ranging from ˜70° C. to 81.0° C. (Table 22). As can be seen in Table 22 and FIG. 32 (thermograms of representative variants), aside from commonly observed single transition profiles (FIG. 32A) humanized 8K22 antibody variants (variants 23796, 23798, 23801 and 23818) exhibited two-state transition profiles (FIG. 32B) and some exhibited weakly pronounced two-state transition profiles (variants 23802, 23814, 23815, 23816, 23817) (FIG. 32B). Whereas such profiles are not generally characteristic of kappa Fabs, they are sometimes Observed and are likely reflective of uncooperative melting of Fab domain, i.e. constant and variable domains unfolding separately.

Humanized variants with the lowest determined Fab Tm values have in common presence of the two amino acid back substitutions to rabbit residues in the FR loop of the humanized light chain, whereas the variants with the highest Tm values have in common the amino acid back substitution to rabbit residue at a position in the variable domain of the humanized light chain that is in contact with the constant domain of that chain. No particular trends were identified in terms of the particular heavy and light chain composition of the variants that could explain the differences in the transition profiles (single or two-state) observed between some of the variants.

### Example 29

### Purity Assessment of Humanized 8K22 Antibodies

The apparent purity of the antibody variants was assessed using mass spectrometry after protein A purification (Example 25) and non-denaturating deglycosylation.

As the antibody variant samples contained Fc N-linked glycans only, the samples were treated with only one enzyme, N-glycosidase F (PNGase-F). The purified samples were de-glycosylated with PNGaseF as follows: 0.1U PNGaseF/μg of antibody in 50 mM Tris-HCl pH 7.0, overnight incubation at 37° C., final protein concentration of 0.48 mg/mL. After deglycosylation, the samples were stored at 4° C. prior to LC-MS analysis.

The deglycosylated protein samples were analyzed by intact LC-MS using an Agilent 1100 HPLC system coupled to an LIQ-Orbitrap™ XL mass spectrometer (ThermoFisher, Waltham, Mass.) (tuned for optimal detection of larger proteins (>50 kDa)) via an Ion Max electrospray source. The samples were injected onto a 2.1×30 mm Koros R2 reverse phase column (Applied Biosystems) and resolved using a 0.1% formic acid aq/acetonitrile (degassed) linear gradient consisting of increasing concentration (20-90%) of acetonitrile. The column was heated to 82.5° C. and solvents were heated pre-column to 80° C. to improve protein peak shape. The cone voltage (source fragmentation setting) was approximately 40 V, the FT resolution setting was 7,500 and the scan range was m/z 400-4,000. The LC-MS system was evaluated for IgG sample analysis using a deglycosylated IgG standard (Waters IgG standard) as well as a deglycosylated mAb standard mix (25:75 half:full sized mAb). For each LC-MS analysis, the mass spectra acquired across the antibody peak (typically 3.6-4.3 minutes) were summed and the entire multiply charged ion envelope (m/z 1,400-4,000) was deconvoluted into a molecular weight profile using the MaxEnt 1 module of MassLynx, the instrument control and data analysis software (Waters, Milford, Mass.). The apparent amount of each antibody species in each sample was determined from peak heights in the resulting molecular weight profiles.

### Results

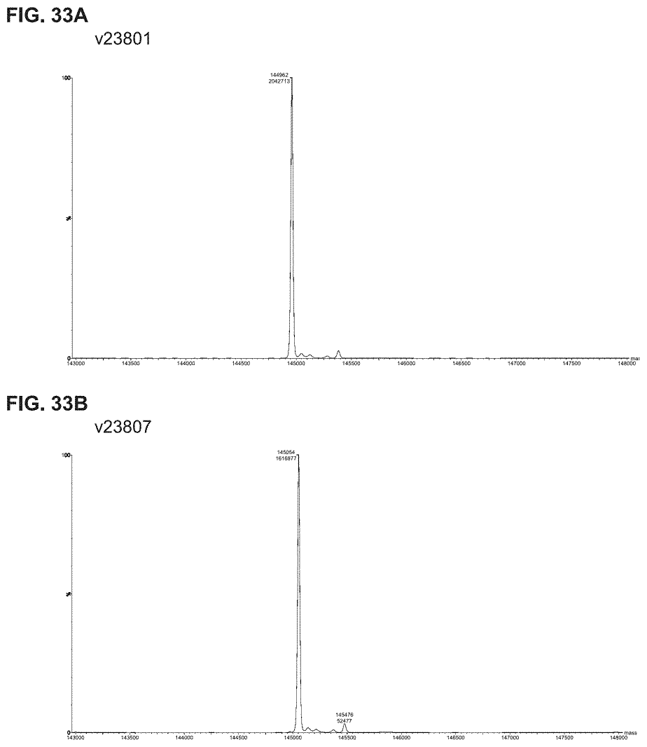

All characterized humanized 8K22 antibody variants were of 100% species purity, exemplified by the LC/MS profile of the two representative humanized antibody samples in FIG. 33. FIG. 33A depicts the LC/MS profile for v23801, while FIG. 33B depicts the LC/MS profile for v23807. In LC/MS profiles of all samples, a peak at ˜+422 Da was present. This peak also was observed in the standard sample run, suggesting it may be a system contaminant and not a sample contaminant.

### Example 30

### Conversion of Fab 8K22 to scFv

The VH and VL sequences of the humanized anti-human folate receptor alpha (anti-hFRα) antibody 8K22 variant 23807 (H4L3), described in Examples 24 and 25, was converted from Fab format to scFv format as described below. This was done to facilitate the production of anti-4-1BB×anti-FRα bispecific antibodies in the 2×1 format described in Example 1 and FIG. 2B.

Design of 8L22 scFvs

A number of 8K22 says were designed in which the order of the VH and VL domains was varied, the length of the linker between the two domains was varied, or the effect of including a stabilizing disulfide bridge was assessed. The 8K22 scFvs were prepared and tested in one-armed antibody format as described in Example 1 and FIG. 1C. For most designs, the 8K22 scFvs were fused to the C-terminus of the Fc, but in some cases the 8K22 scFvs were fused to the N-terminus of the Fc. A summary of the 8K22 scFvs designed is found in Table 23. The sequences for the 8K22 say portion of each variant is found in Table 27 in Example 32.

TABLE 23
scFv conversion

| | | | | Di- | Fc |
|---|---|---|---|---|---|
| Antibody | | Orien- | | sul- | Fu- |
| Variant | Description | tation | Linker | fide | sion |
| v29675 | N-term Parent Fab | Parent | | | N- |
| | | Fab | | | |
| v29686 | C-term Parent Fab | C-term | | | C- |
| | | Fab | | | |
| v29676 | N-term VL-(short)-VH | VL-VH | (G₄S)₃ | | N- |
| v29677 | N-term VL-(long)-VH | VL-VH | (G₄S)₄ | | N- |
| v29678 | C-term VL-(short)-VH | VL-VH | (G₄S)₃ | | C- |
| v29679 | C-term VH-(short)-VL | VH-VL | (G₄S)₃ | | C- |
| v29680 | C-term VL-(long)-VH | VL-VH | (G₄S)₄ | | C- |
| v29681 | C-term VH-(long)-VL | VH-VL | (G₄S)₄ | | C- |
| v29682 | C-term VL-(short)-VH + | VL-VH | (G₄S)₃ | Yes | C- |
| | disulphide | | | | |
| v29683 | C-term VH-(short)-VL + | VH-VL | (G₄S)₃ | Yes | C- |
| | disulphide | | | | |
| v29684 | C-term VL-(long)-VH + | VL-VH | (G₄S)₄ | Yes | C- |
| | disulphide | | | | |
| v29685 | C-term VH-(long)-VL + | VH-VL | (G₄S)₄ | Yes | C- |
| | disulphide | | | | |

In more detail, humanized 8K22 variable light (VL) and variable heavy (VH) domains were converted to an scFv as follows: The VL (SEQ ID NO: 316) and VH (SEQ ID NO: 310) amino acid sequences were generated according to Kabat definitions. The VL and VH sequences were combined as a single sequence separated by a short linker sequence. The linker sequence was either (G₄S)₃ (short, GGGGSGGGGSGGGGS. SEQ NO:320) or (G₄S)₄ (long, GGGGSGGGGSGGGGSGGGGS, SEQ NO:321). The order of the domains was either VL-linker-VH or VH-linker-VL (see “Orientation” column in Table 23) where VL-VH indicates the VL sequence precedes the VH sequence and is connected by a short linker. VH-VL indicates the VH sequence precedes the VL sequence and is connected by a short linker. A stabilization disulfide between the VL and VH was introduced in some variants at position VL-G100C and VH-G44C according to the Kabat numbering system. This is denoted in Table 23 under the Disulfide column with Yes. The scFv designs used are described in table 23. For example, v29683 C-term VH-(short)-VL+disulphide is fused to the C-terminus of the Fc by the VH domain, followed by a (G₄S)₃ linker, and VL domain. The VH and VL domain contain a disulfide bond at VL-G100C and VH-G44C. The variants were constructed in a One-armed format generated using the heterodimeric Fc design described in Example 17.

Each of the 8K22 scFv sequences described in Table 23 were fused to an Fc sequence having the Het FcA mutations, as described in Example 17, at either the N-terminus or C-terminus. If fused to the N-terminus of Het FcA a short Ala-Ala linker was included between the scFv and the hinge of Het FcA. If fused to the C-terminus of the Het FcA a short Gly-Gly-Gly-Gly (SEQ ID NO:336) linker was included between the Het FcA and the scFv. in all constructs the cysteine located in the upper hinge at position Kabat: 233 was mutated to SER. These sequences were reverse translated to DNA, codon optimized for mammalian expression, and gene synthesized.

All parent (humanized 8K22) and scFv converted sequences were preceded by the artificially designed signal peptide sequence MRPTWAWWLFLVLLLALWAPARG [SEQ ID NO:1] (ref: Barash S et al., Biochem and Biophys Res. Comm, 2002; 294, 835-842). For all parental and scFv converted chains, vector inserts were prepared as described in Example 1 and cloned into the pTT5 expression vector.

### Example 31

### Production of Fc-Fused 8K22 ScFv Variants

Variants described in Example 30 were prepared under transient mammalian expression conditions and subsequently purified and characterized for stability and antigen binding. Samples of Fc-fused 8K22 scFv variants post protein-A were subjected to UPLC-SEC to assess the amount of high molecular species. Further, thermal stability of 8K22 scFv in Fc-fused 8K22 scFv variants was assessed by differential scanning calorimetry (DSC) as described below. This was performed to identify the optimal design for an 8K22 scFv.

### Method

The two different heavy chains in Fc-fused scFv variants and heavy and light chains in Fab containing (29675 and 29686) antibody variants were co-expressed in 200 mL CHO cultures and purified as described in Example 1. Following protein-A purification, purity of samples was assessed by non-reducing and reducing High Throughput Protein Express assay (LabChip) as described in Example 1.

Post protein-A purification, samples were either buffer exchanged into DPBS (Dulbecco's PBS) and aseptically filtered or, depending on their homogeneity assessed by UPLC-SEC, subjected to SEC purification as described in Example 1. UPLC-SEC was performed as described in Example 1.

Final purified samples were analyzed with DSC to determine their thermal stability. DSC experiments were performed as described in Example 14.

### Results

Yields of variant post protein-A were in the range of 85-109 mg/L for the Fc-fused 8K22 scFv variants. Non-reducing and reducing LabChip post protein-A reflected a predominant species of the desired molecular weight.

As can be seen on Table 24, UPLC-SEC profiles showed a variety of homogeneity in the different variants. Some variants contained high molecular weight (HWM) species (ie dimers, trimers, higher order aggregates) of up to 56%, v29676. Variants with a (G₄S)₄ linker (v29677, v29680 and v29681) had the lowest measured HMW species, 21-34%. With v29680 having the lowest measured HMW species at 21.8% All samples had the HMW species removed with preparative SEC.

TABLE 24
High molecular weight species in Fc-fused 8K22 scFv samples

| Antibody | | Yield post | HWM species |
|---|---|---|---|
| Variant | Description | Protein A (mg/L) | Post Protein A |
| v29675 | N-term Parent Fab | 100.3 | 10.1% |
| v29676 | N-term VL-(short)-VH | 86 | 56.5% |
| v29677 | N-term VL-(long)-VH | 85.3 | 34.4% |
| v29678 | C-term VL-(short)-VH | 102.4 | 55.4% |
| v29679 | C-term VH-(short)-VL | 108.3 | 43.6% |
| v29680 | C-term VL-(long)-VH | 100.7 | 21.8% |
| v29681 | C-term VH-(long)-VL | 105.8 | 27.5% |
| v29682 | C-term VL-(short)-VH + | 98.4 | 48.5% |
| | disulphide | | |
| v29683 | C-term VH-(short)-VL + | 103.3 | 48.6% |
| | disulphide | | |
| v29684 | C-term VL-(long)-VH + | 107.4 | 40.2% |
| | disulphide | | |
| v29685 | C-term VH-(long)-VL + | 96.8 | 43.9% |
| | disulphide | | |

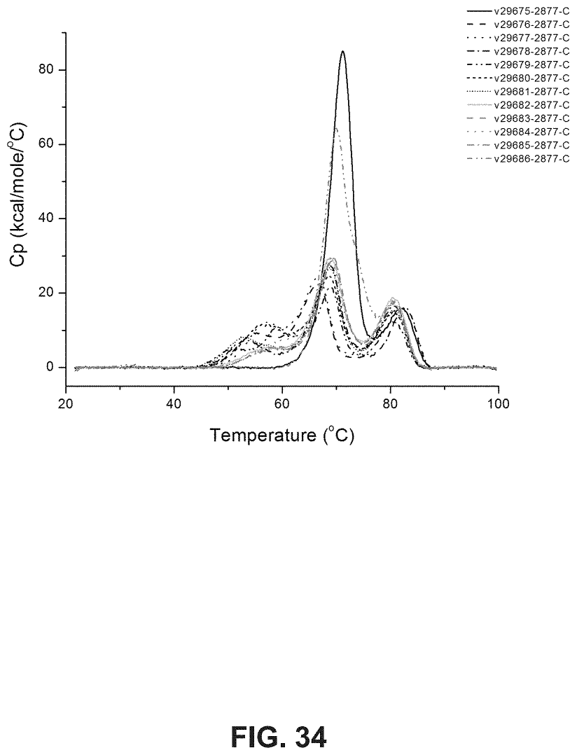

FIG. 34 shows the DSC thermograms of the Fc-fused 8K22 scFv antibodies tested. The Tm values, corresponding to the scFv portion of the Ab, determined from the thermograms are shown in Table 25 below.

TABLE 25
Thermal stability of 8K22 scFv in Fc-fused variants

| Antibody | | |
|---|---|---|
| Variant | Description | Tm (° C.) |
| v29675 | N-term Parent Fab | 71.5 |
| V29686 | C-term Parent Fab | 74.7 |
| v29676 | N-term VL-(short)-VH | 54.8 |
| v29677 | N-term VL-(long)-VH | 57.0 |
| v29678 | C-term VL-(short)-VH | 53.5 |
| v29679 | C-term VH-(short)-VL | 50.3 |
| v29680 | C-term VL-(long)-VH | 56.4 |
| v29681 | C-term VH-(long)-VL | 52.9 |
| v29682 | C-term VL-(short)-VH + | 58.4 |
| | disulfide | |
| v29683 | C-term VH-(short)-VL + | 60.4 |
| | disulfide | |
| v29684 | C-term VL-(long)-VH + | 60.0 |
| | disulfide | |
| v29685 | C-term VH-(long)-VL + | 59.3 |
| | disulfide | |

Each peak on the thermogram corresponds to a thermal transition. There are three expected thermal transitions: Fab/scFv, CH2 (˜71° C.) and CH3 (˜80° C.). The transition of the Fab reflects cooperative melting of the VH-VL and CH1-CL domains. The expected transition of the say would correspond to the melting of the VH-VL domains. Some scFvs do not undergo cooperative melting and two transitions were observed. In these cases the lower Tm is reported in Table 25. The 8K22 Fab transition overlaps with the CH2 domain transition and therefore only 2 transition peaks are observed in the thermogram for 8K22 parent Fab antibody variants. As can be seen in FIG. 34, three distinct transitions can be observed for all variants which contain a 8K22 scFv. The Tm of the 8K22 scFv was 10-15° C. lower than the parent Fab antibody. The engineered disulfide bond increases the Tm of the scFv between 1-6.5° C. Of the scFvs having a disulfide bond, the scFv of v29683 and v29684 had the highest thermal stability. Of the scFvs without a disulfide bond, the scFv of v29679 had the highest thermal stability.

### Example 32

### Binding of Fc-Fused 8K22 scFv Antibodies to Human FRα by Bio-Layer Interferometry

To assess the ability of the Fc-fused 8K22 scFv antibodies to retain Fab-like binding to human FRα, the affinity of scFv converted antibodies described in Example 31 were compared to the parental chimeric antibodies described in Examples 24 and 25 by Bio-layer interferometry (BLI).

Post SEC protein material described in Example 31 was assessed for binding to human FRα. Binding was measured by Bio-laver interferometry (BLI) using the Octet RED 96 (ForteBio) as described in Example 27. All parameters remained the same except for the dissociation phase which was recorded for 1500 s.

### Results

The KDs measured for each Fc-fused 8K22 scFv variant are provided in Table 26.

TABLE 26
Binding of Fc-fused 8K22 scFv variants to hFRα

| Antibody | | Binding Results (n = 2) | |
|---|---|---|---|
| Variant | Description | KD(M) | STDev KD (M) |
| v29675 | N-term Parent Fab | 4.1E−9 | 8E−11 |
| v29676 | N-term VL-(short)-VH | 3.9E−9 | 9E−11 |
| v29677 | N-term VL-(long)-VH | 5.0E−9 | 1E−10 |
| v29678 | C-term VL-(short)-VH | 5.1E−9 | 1E−10 |
| v29679 | C-term VH-(short)-VL | 3.9E−10 | 5E−11 |
| v29680 | C-term VL-(long)-VH | 1.8E−9 | 7E−11 |
| v29681 | C-term VH-(long)-VL | 2.6E−9 | 7E−11 |
| v29682 | C-term VL-(short)-VH + | 2.1E−9 | 8E−11 |
| | disulfide | | |
| v29683 | C-term VH-(short)-VL + | 1.1E−9 | 5E−11 |
| | disulfide | | |
| v29684 | C-term VL-(long)-VH + | 8.8E−10 | 1E−10 |
| | disulfide | | |
| v29685 | C-term VH-(long)-VL + | 4.2E−9 | 4E−10 |
| | disulfide | | |

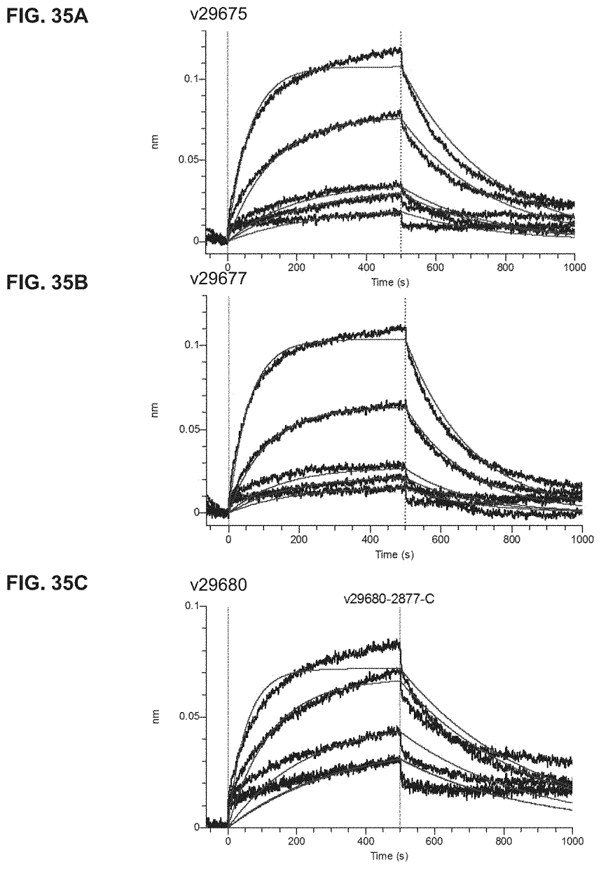

As can be seen from Table 26, the BLI binding assay performed on the Fc-fused 8K22 scFv variants revealed that all say variants bound to hFRα with an affinity that was within 2-fold of the Parent Fab antibody. FIG. 35 provides BLI sensorgrams for the Parent Fab antibody (FIG. 35A) and two representative Fc-fused scFv variants (FIG. 35B and 35C) that were able to bind human FRα. These results suggest that the conversion to the scFv format and addition of the disulfide bond did not impact the binding to the antigen and that the location of the scFv, N-term or C-term, also had no effect on binding.

TABLE 27
8K22 scFv amino acid sequences of variants

| SEQ | | |
|---|---|---|
| ID | | |
| NO: | Variant | 8K22 scFv Sequence |
| 334 | v29676, | DIQMTQSPSSLSASVGDRVTITCQASQSIYSSLAWYQQKP |
| | v29678 | GKAPKLLIYDASHLASGVPSRFSGSRYGTDFTLTISSVQP |
| | | EDFATYYCQGGWYSSAATYVPNTFGGGTKVEVKGGGGSGG |
| | | GGSGGGGSEVQLVESGGGLVQPGGSLRLSCAASGFTISNN |
| | | YYMCWVRQAPGKGLEWIACIYGGISGRTYYADSAKGRFTI |
| | | SKDSSNTVYLQMNSLRAEDTAVYYCVRGYVGTSNLWGQGT |
| | | LVTVSS |
| 335 | v29677, | DIQMTQSPSSLSASVGDRVTITCQASQSIYSSLAWYQQKP |
| | v29680 | GKAPKLLIYDASHLASGVPSRFSGSRYGTDFTLTISSVQP |
| | | EDFATYYCQGGWYSSAATYVPNTFGGGTKVEVKGGSGGGS |
| | | GGGSGGGSGGGSGEVQLVESGGGLVQPGGSLRLSCAASGF |
| | | TISNNYYMCWVRQAPGKGLEWIACIYGGISGRTYYADSAK |
| | | GRFTISKDSSNTVYLQMNSLRAEDTAVYYCVRGYVGTSNL |
| | | WGQGTLVTVSS |
| 337 | v29679 | EVQLVESGGGLVQPGGSLRLSCAASGFTISNNYYMCWVRQ |
| | | APGKGLEWIACIYGGISGRTYYADSAKGRFTISKDSSNTV |
| | | YLQMNSLRAEDTAVYYCVRGYVGTSNLWGQGTLVTVSSGG |
| | | GGSGGGGSGGGGSDIQMTQSPSSLSASVGDRVTITCQASQ |
| | | SIYSSLAWYQQKPGKAPKLLIYDASHLASGVPSRFSGSRY |
| | | GTDFTLTISSVQPEDFATYYCQGGWYSSAATYVPNTFGGG |
| | | TKVEVK |
| 339 | v29681 | EVQLVESGGGLVQPGGSLRLSCAASGFTISNNYYMCWVRQ |
| | | APGKGLEWIACIYGGISGRTYYADSAKGRFTISKDSSNTV |
| | | YLQMNSLRAEDTAVYYCVRGYVGTSNLWGQGTLVTVSSGG |
| | | SGGGSGGGSGGGSGGGSGDIQMTQSPSSLSASVGDRVTIT |
| | | CQASQSIYSSLAWYQQKPGKAPKLLIYDASHLASGVPSRF |
| | | SGSRYGTDFTLTISSVQPEDFATYYCQGGWYSSAATYVPN |
| | | TFGGGTKVEVK |
| 340 | v29682 | DIQMTQSPSSLSASVGDRVTITCQASQSIYSSLAWYQQKP |
| | | GKPKLLIYDASHLASGVPSRFSGSRYGTDFTLTISSVQPE |
| | | DFATYYCQGGWYSSAATYVPNTFGCGTKVEVKGGGGSGGG |
| | | GSGGGGSEVQLVESGGGLVQPGGSLRLSCAASGFTISNNY |
| | | YMCWVRQAPGKCLEWIACIYGGISGRTYYADSAKGRFTIS |
| | | KDSSNTVYLQMNSLRAEDTAVYYCVRGYVGTSNLWGQGTL |
| | | VTVSS |
| 341 | v29683 | EVQLVESGGGLVQPGGSLRLSCAASGFTISNNYYMCWVRQ |
| | | APGKCLEWIACIYGGISGRTYYADSAKGRFTISKDSSNTV |
| | | YLQMNSLRAEDTAVYYCVRGYVGTSNLWGQGTLVTVSSGG |
| | | GGSGGGGSGGGGSDIQMTQSPSSLSASVGDRVTITCQASQ |
| | | SIYSSLAWYQQKPGKAPKLLIYDASHLASGVPSRFSGSRY |
| | | GTDFTLTISSVQPEDFATYYCQGGWYSSAATYVPNTFGCG |
| | | TKVEVK |
| 342 | v29684 | DIQMTQSPSSLSASVGDRVTITCQASQSIYSSLAWYQQKP |
| | | GKAPKLLIYDASHLASGVPSRFSGSRYGTDFTLTISSVQP |
| | | EDFATYYCQGGWYSSAATYVPNTFGCGTKVEVKGGSGGGS |
| | | GGGSGGGSGGGSGEVQLVESGGGLVQPGGSLRLSCAASGF |
| | | TISNNYYMCWVRQAPGKCLEWIACIYGGISGRTYYADSAK |
| | | GRFTISKDSSNTVYLQMNSLRAEDTAVYYCVRGYVGTSNL |
| | | WGQGTLVTVSS |
| 343 | v29685 | EVQLVESGGGLVQPGGSLRLSCAASGFTISNNYYMCWVRQ |
| | | APGKCLEWIACIYGGISGRTYYADSAKGRFTISKDSSNTV |
| | | YLQMNSLRAEDTAVYYCVRGYVGTSNLWGQGTLVTVSSGG |
| | | SGGGSGGGSGGGSGGGSGDIQMTQSPSSLSASVGDRVTIT |
| | | CQASQSIYSSLAWYQQKPGKAPKLLIYDASHLASGVPSRF |
| | | SGSRYGTDFTLTISSVQPEDFATYYCQGGWYSSAATYVPN |
| | | TFGCGTKVEVK |

### Example 33

### Generation of 4-1BB×FRα Bispecific Antibodies Utilising Humanized Paratopes

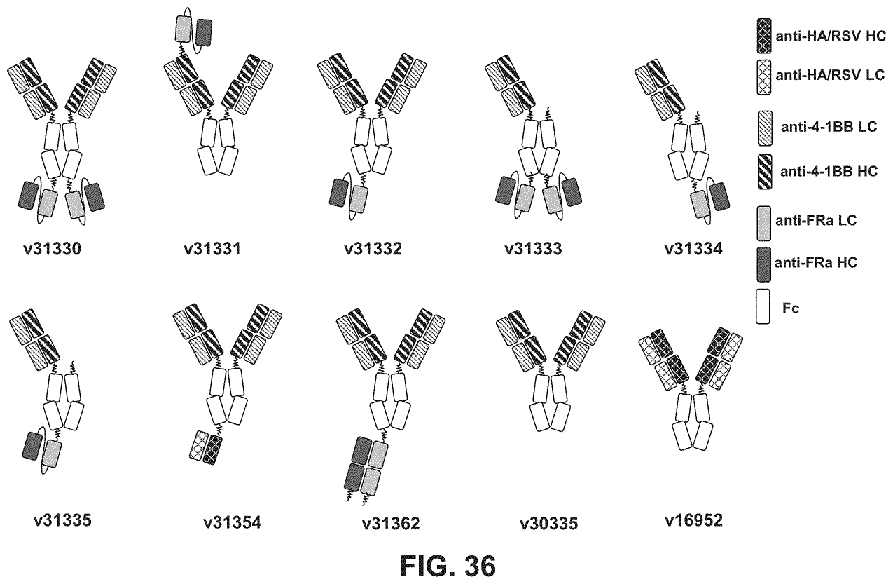

Additional 4-1BB×FRα antibody constructs (antibodies) v31330, v31331, v31332, v31333, v31334, and v31335 were designed to assess the effect of format on the ability of these 4-1BB×I FRα bispecific constructs to conditionally activate 4-1BB. FIG. 36 provides a representation of the formats of the antibodies tested in this example. These 4-1BB×FRα antibody constructs were prepared using the humanised anti-4-1BB paratope 1G1 corresponding to variant 28684 (H1L2), described in Example 10, and the anti-FRα humanised paratope 8K22 based on variant 23807 (H4L3) converted into an scFv, described in Example 30. Table X1 identifies the clones that make up each of the antibody constructs. The polypeptide sequences of each clone can be found in Table Y1.

Antibodies were expressed and purified as described in Example 1. Purified antibodies were then tested as described in subsequent examples.

### Example 34

### Activity of Humanised 4-1BB×FRα Bispecifics in a Primary T Cell Tumour Co-Culture Assay

The antibodies described in the previous example were tested in a primary T cell:tumour co-culture assay to assess their ability to activate 4-1:BB, according to methods previously described in Example 22. Briefly, CD8+ T cells were placed into wells of a 384-well plate with IGROV1 tumour cells and aAPC/CHO-K1 cells and samples of antibodies.

After four days, supernatants were taken and the concentration of IFNγ measured by MesoScale Discovery (MSD) U-Plex IFNγ 384-well assay kit (Meso Scale Diagnostics, Rockville, Md.). Prior to use, the MSD plates were blocked by adding 50 μl Diluent 100 (Meso Scale Diagnostics, Rockville, Md.) to wells of a MA6000 384-well SA plate for 30 minutes at room temperature. The blocking solution was then removed and 100 μl of capture antibody added to each well (228 μl of biotylated capture antibody diluted in 3.77 ml Diluent 100). The plate was left at 4° C. overnight, and then washed three times with PBS 0.05% Tween-20.

Tissue culture supernatant samples were diluted 1:20 with Diluent 43 (Meso Scale Diagnostics) and 5 μL of the diluted supernatant was placed into wells containing 5 μL of Diluent 43. The plate was left at room temperature for an hour to allow binding, then washed three times with PBS 0.05% Tween-20. SULFO-TAG IFNγ detection antibody (MesoScale Diagnostics) was diluted 100× in Diluent 3 and 10 μl of the resulting solution was added to each well, and the plate incubated for a further hour. The plate was then washed three times in PBS 0,05% Tween-20. 40 μl of MSD GOLD Read buffer was added to each well, and the plate read on a MesoSector 8600 instrument (MesoScale Diagonistics). The amount of IFNγ in each sample was calculated according to a standard curve generated from recombinant IFNγ (R&D System).

### Results

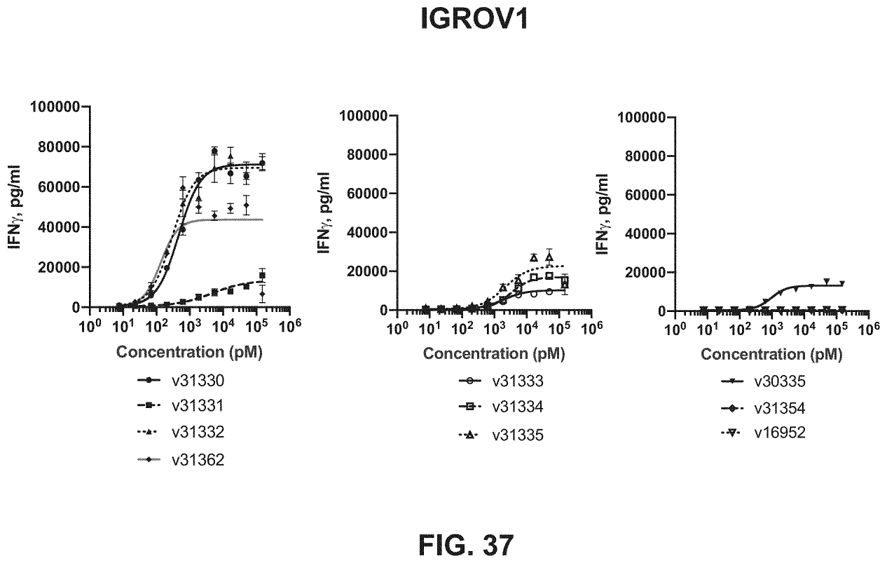

As can be seen from FIG. 37, 4-1BB×FRα bispecific antibodies showed induction of IFNγ in co-culture with IGROV1 cells. Samples v31332, v31362 and v31330 which have two 4-1BB binding domains showed both the highest potency and highest activity, as shown by the total production of IFNα, v31332, v31362 and v31330 also showed higher activity than v30335, a monospecific 4-1BB antibody that is not reliant on Fc-mediated crosslinking. Samples with only a single 4-1BB binding arm (v31333, v31334 and v31335) induced production of IFNγ in dose-dependent manner, but total production of cytokine was lower than seen for samples with two 4-1BB binding arms. v31331, despite having two 4-1BB binding domains, showed activity similar to antibodies with a single 4-1BB binding domain (v31333, v31334 and v31335), suggesting that the 4-1BB and FRα binding domains on the same arm of v31331 may result in a geometry that prevents engagement of both 4-1BB and FRα simultaneously, reducing activity to that seen with a single 4-1BB arm.

Two antibodies were used as control antibodies for non-specific activity. v16952, which does not bind mammalian proteins, and v31354, which contains the same 4-1BB binding domains and same format as v3I332 but does not bind FRα did not show any activity in this experiment, suggesting that the IFNg production seen in this experiment was due to 4-1BB costimulation and clustering of the 4-1BB antibody via FRα.

### Example 35: Activation of 44BB by 4-1BB×FRα Bispecific Antibodies in Co-Culture with Lung and Ovarian Cell Lines

The 4-1BB×FRα antibodies described in Example 33 were assessed for induction of 4-1BB signalling in co-culture with lung and ovarian cancer cell lines. The assay was carried out using the NFκB reporter gene assay described in Example 3, but with the tumour cell lines described in Table 28 below:

TABLE 28
Ovarian and lung cell lines

| Cell line | Source | Type | FRα level |
|---|---|---|---|
| IGROV1 | NCI | Ovarian endometrioid | FRα^{high} |
| | | Adenocarcinoma | |
| OVCAR3 | ATCC | Ovarian serous | FRα^{mid} |
| | | adenocarcinoma | |
| OVKATE | JCRB | Ovarian serous | FRα^{mid} |
| | | adenocarcinoma | |
| NCI-H441 | ATCC | Papillary lung | FRα^{mid} |
| | | adenocarcinoma | |
| NCI-H661 | ATCC | Large cell lung carcinoma | FRα^{low/neg} |
| NCI-H1975 | ATCC | Lung adenocarcinoma | FRα^{high} and FRα^{low} |
| | | | (heterogeneous) |
| HCC827 | ATCC | Lung adenocarcinoma | FRα^{low} |
| A431 | ATCC | Vulvar squamous cell | FRα^{low} |
| | | carcinoma | |

Cell lines were obtained from ATCC (Manassas, Va., USA), with the exception of OVKATE (Japanese Collection of Research Bioresources Cell Bank, Osaka, Japan) and IGROV1 (National Cancer institute, Bethesda, Md., USA). Cells were assigned as FRα^{high}, FRα^{mid} and FRα^{low} based on binding of v17717 directly conjugated to Alexa647 by flow cytometry, as described in Example 18.

### Results

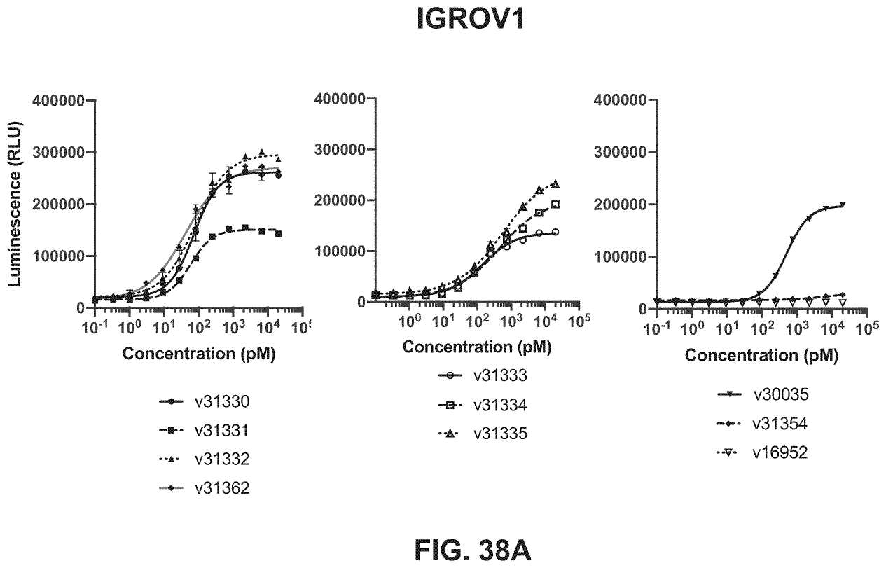

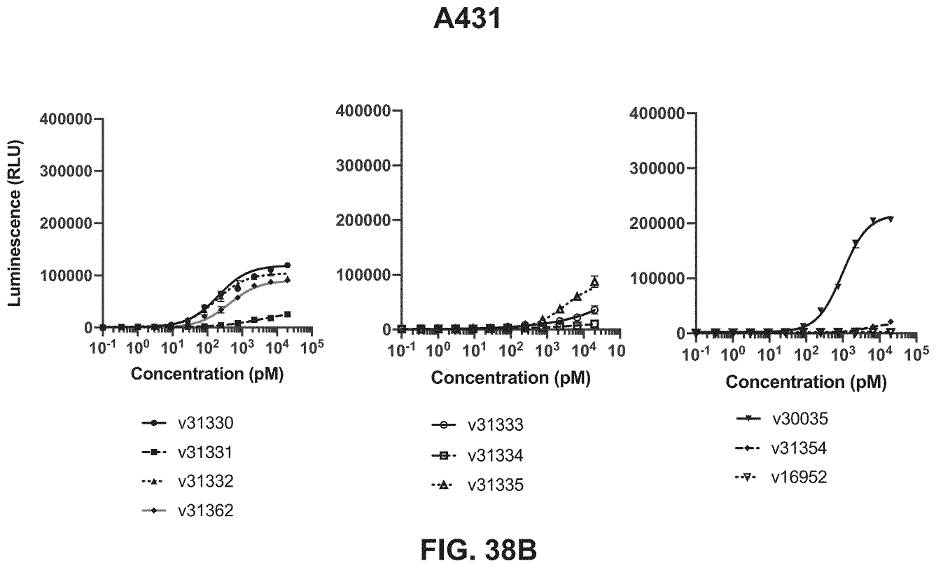

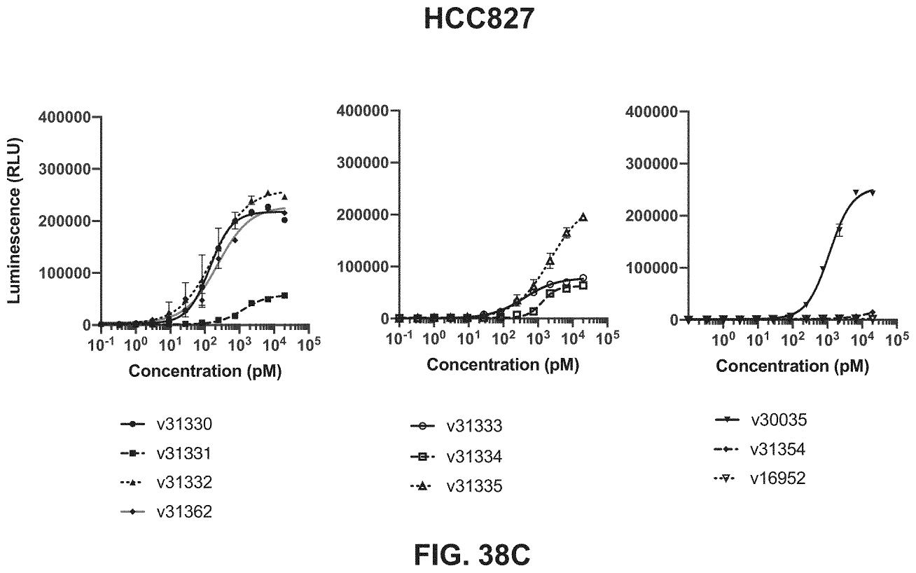

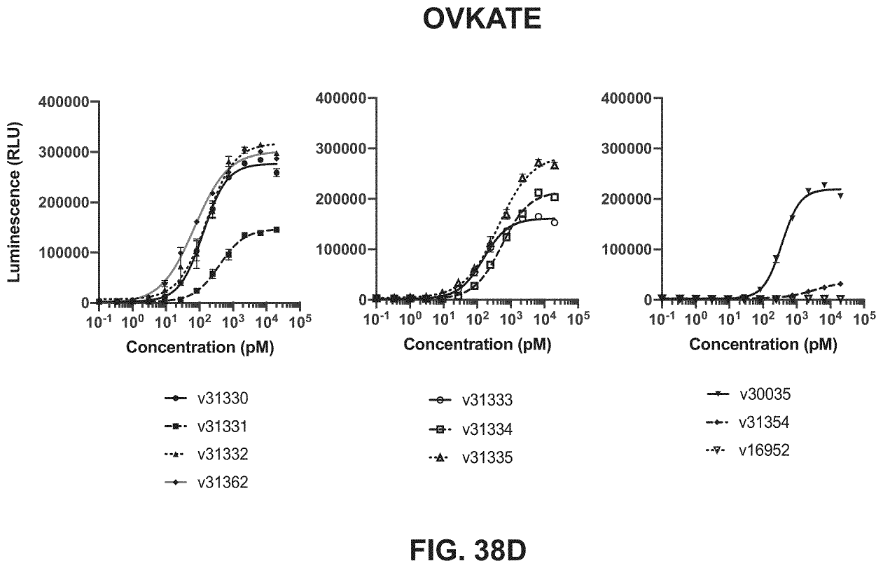

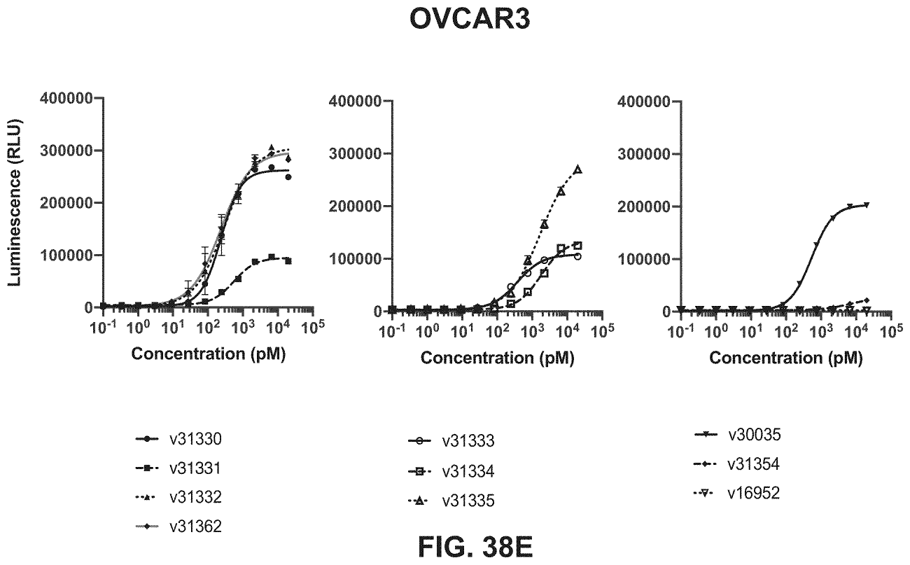

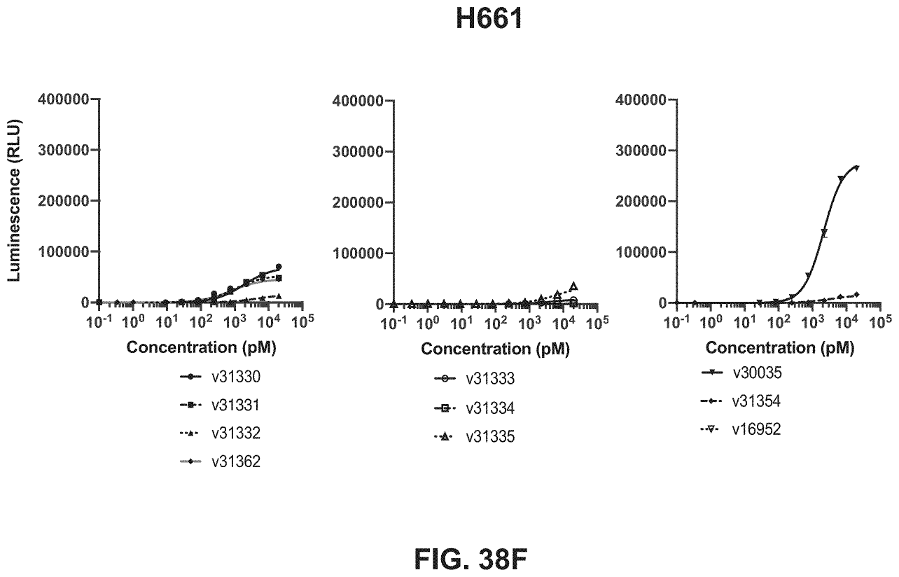

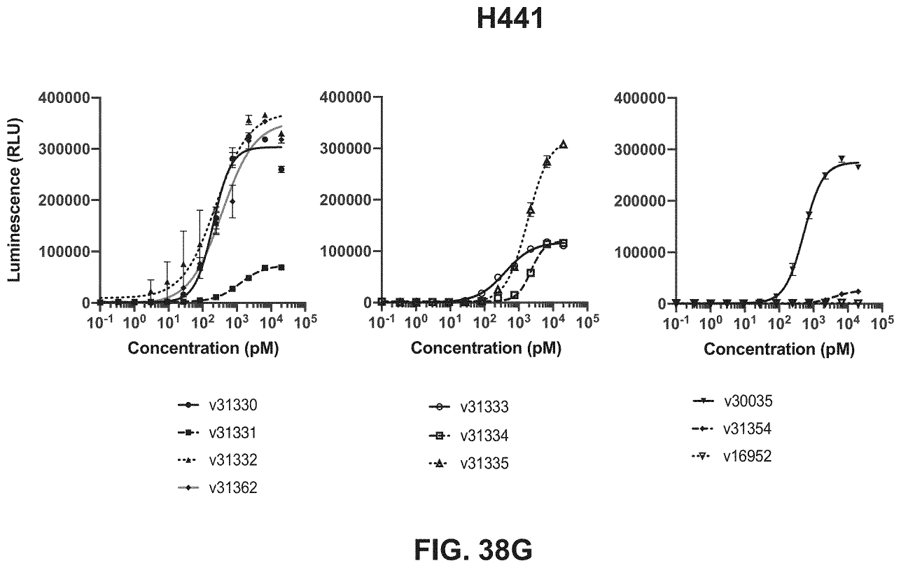

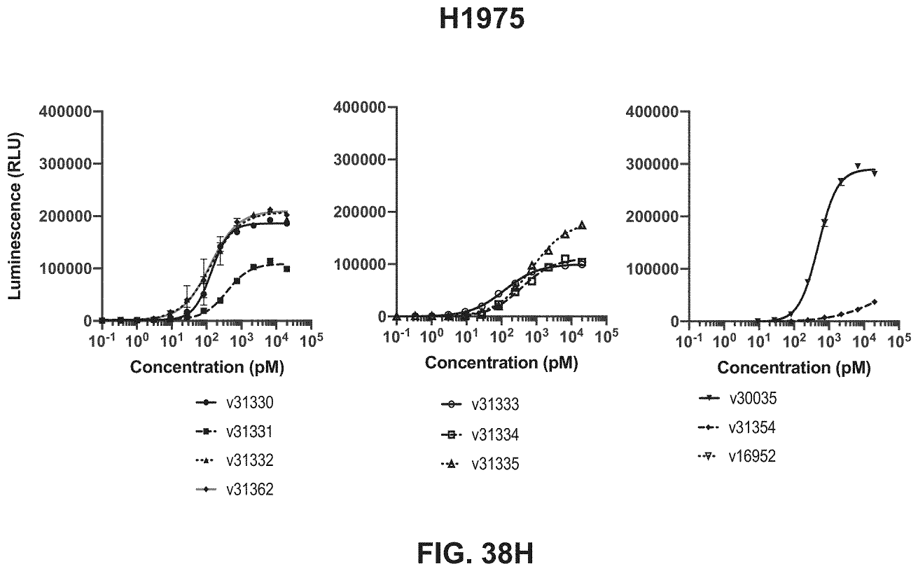

Similar to previous experiments, IGROV1 cells in co-culture with Jurkat T cells with a 4-1BB NFκB reporter system showed activation by 4-1BB×FRα antibodies in a dose-dependent manner (FIG. 38A). 4-1BB×FRα antibodies with two 4-1BB binding domains v31332, v31330 and v31362) showed greater activity than antibodies only having a single 4-1BB binding domain (v31333, v31334 and v31335) (FIG. 38A-H). The exception was v31331, which has two 4-1BB binding domains but had similar activity to v31333, v31334 and v31335. v31331 may only be able to engage a single 4-1BB on the Jurkat at the same time as the FRα on the tumour cell, potentially due to the proximity of the 4-1BB and FRα binding pockets.

Activity on cell lines derived from patients with either Ovarian or Lung cancers were also tested. These cell lines also expressed different levels of FRα, enabling the examination of the effect of FRα level on activity. The maximal activity and potency were higher when the antibodies were in co-culture with FRα^{high} cells and correlated with levels of FRα on the surface of the tumour cells (FIG. 38A-H). However, the relative activity on the antibodies did not change, with the highest potency and activity seen from v31332, v31330 and v31362 compared to the other antibodies. Activity was also seen in co-culture with FRα-positive Ovarian and Lung cancer cell lines of diverse origin. On FRα^{low} cell lines, activity could be seen but was lower than seen with the monospecific 4-1BB antibody v30335.

### Example 36

### Ability of Selected Anti-4-1BB Paratopes to Bind to 4-1BB from Cynomolgus (Cyno) Monkeys

The ability of select humanized antibodies to bind cyno 4-1BB by SPR was assessed and compared to that of the parent mouse paratopes 1C8, 1G1, and 5G8. Cyno cross-reactivity of these antibodies had been assessed using a homogeneous cell binding assay as described in Example 9; in this experiment cyno cross-reactivity was assessed using SPR. The SPR method used was similar to the one described in Example 2, except that SEC-purified Cyno 4-1BB-His (Acro Biosystems) was used in place of the human 4-1BB. The antibodies tested are described in Table 29 below:

TABLE 29
Antibodies tested

| Variant | Paratope | Description |
|---|---|---|
| 20022 | 1C8 | Mouse VH and VL |
| 20023 | 1G1 | sequences, Example 6, |
| 20036 | 5G8 | Table 13 |
| 28727 | 1C8 | Humanized VH and VL |
| 28684 | 1G1 | sequences, Example 10, |
| 28704 | 5G8 | Table 14 |

### Results

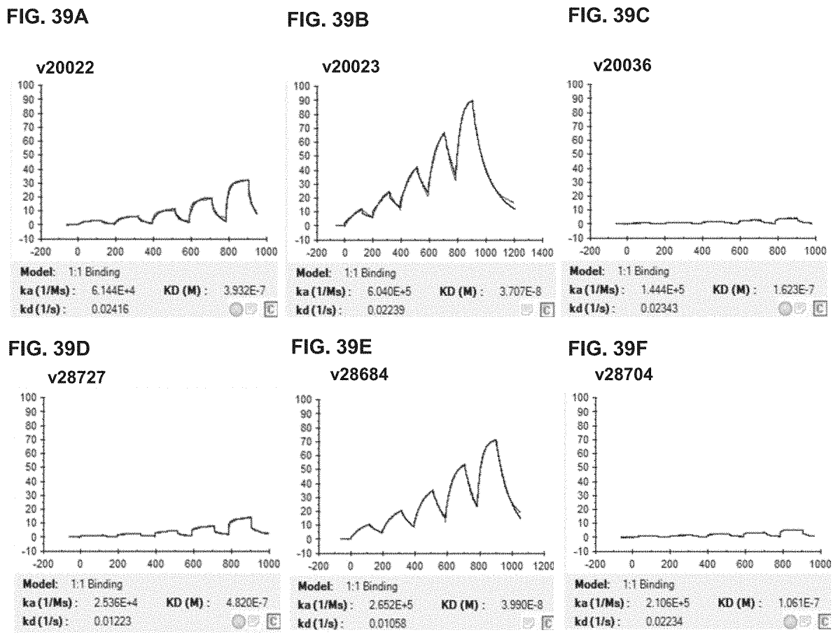

Binding of cyno 4-1BB to both v20023 and v28684 was similar, suggesting that the 1G1 paratope bound cyno 4-1BB before and after humanization. The 1C8 paratope, similar to seen with human 4-1BB, lost some binding after humanization as can be seen in the difference between v20022 and v28727. In contrast to FIG. 11A where v20036 appeared to bind cyno 4-1 BB, by SPR v20036 binds poorly to cyno 4-1BB. The discrepancy between FIG. 11A and the data in FIG. 39 is likely due to the inability of the method used in FIG. 11A to discriminate between antibodies which bind well and antibodies which bind poorly to cyno 4-1BB.

Tables 13-22

TABLE 13
Recovered anti-human 4-1BB antibody sequences

| | Anti- | | |
|---|---|---|---|
| Variant | body | VH | VL |
| 20020 | 1B2 | QVQLQQPGAELVKPGASVKLSCKASGYT | DVVMTQTPLSLPVSLGDQASISCRSSQ |
| | | FTSFWINWVKQRPGQGLEWIGNIYPDSS | SIVHSNGNTYLEWYLQKPGQSPKLLIY |
| | | STNYNEKFKNKATLTVDTSSSTAYMQLS | KVSNRFSGVPDRFSARGSGTDFSLKIS |
| | | SLTSDDSAVYFCARSLTFDYWGQGTTLT | RVEAEDLGVYYCFQGSHVPYTEGGGTK |
| | | VSS [SEQ ID NO: 3] | LEIK [SEQ ID NO: 4] |
| 20021 | 1C3 | QVQLKQSGPGLVQPSQSLSITCTVSGFS | DVVMTQTPLSLPVSLGDQASISCRSSQ |
| | | LTSYGVHWVRQSPGKGLEWLGVIWRGGS | SIVHSNGNTYLEWYLQKPGQSPKLLIY |
| | | TDYNAAFISRLSISKDNSKSQVFFKMNS | KVSNRFSGVPDRFSARGSGTDFSLKIS |
| | | LQADDTAIYYCARENYDYDEFAYWGQGT | RVEAEDLGVYYCFQGSHVPYTEGGGTK |
| | | LVTVSA [SEQ ID NO: 5] | LEIK [SEQ ID NO: 6] |
| 20022 | 1C8 | QVQLKESGPGLVAPSQSLSITCTVSGFS | DIQMTQSTSSLSASVGDRVTISCSASQ |
| | | LTTYAISWVRQPPGKGLEWLGIIWPGGG | GISNYLNWYQQKPDGTVKLLIYYTSSL |
| | | TNYNSALKSRLSISKDNSRSQVFLKMNS | HSGVPSRFSGSGSGTDYSLTISNLEPE |
| | | LQTDDTARYYCARGAGTWYFDVWGAGTT | DIATYYCQQYSKFPWTFGGGTKLEIK |
| | | VTVSS [SEQ ID NO: 7] | [SEQ ID NO: 8] |
| 20023 | 1G1 | EVQLVESGGGLVKPGGSRKLSCAASGFT | NIVMTQSPKSMSMSVGERVTLSCKASE |
| | | FSEFGMHWVRQAPDKGLKWVAYISSGGS | NVGSYVSWYOQKPEKSPKLLIYGASNR |
| | | TIYYADTVKGRFTISRDNAKNTLFLQMT | YTGVPDRFTGSGSATDFTLTISSVQAE |
| | | SLRSEDTAMYYCARDWVDYWGQGTTLTV | DLADYHCGQSYSYPLTFGAGTKLELK |
| | | SS [SEQ ID NO: 9] | [SEQ ID NO: 10] |
| 20024 | 2A7 | EVQLQQSGPELVKPGASVKMSCKASGYT | DIVMTQSHKFMSTSVGDRVSITCKASQ |
| | | FTSYVMHWVKQKPGQGLEWIGYINPYND | DVGTAVGWYQQKLGQSPKLLIYWASTR |
| | | GTKYNEKFKGKATLTSDKSSSTAYMELS | RTGVPDRFTGSGSGTDFTLTISNVQSE |
| | | SLTSEDSAVYYCARLGSRGTWFAYWGQG | DLADYFCQQYSSYPLTFGAGTKLELK |
| | | TLVTVSA [SEQ ID NO: 11] | [SEQ ID NO: 12] |
| 20025 | 2E8 | QVQLKQSGPGLVQPSQSLSITCTVSGFS | DIVMTQSQKFMSTSVGDRVSVTCKASQ |
| | | LTSYGVHWVRQSPGKGLEWLGVIWSGGS | NVGTNVAWYQQKPGQSRTVLIYSASYR |
| | | TDYNAAFISRLSISKDNSKSQVFFKMNS | HSGVPDRFTGSGSGTDFTLTISNVQSE |
| | | LQADDTAIYYCARNPLTATVMDYWGQGT | DLAEYFCQQYNSYPLTEGTGTKLELK |
| | | SVTVSS [SEQ ID NO: 13] | [SEQ ID NO: 14] |
| 20026 | 2H9 | QVQLQQPGAELVRPGASVKLSCKASGYP | DIVMTQAAFSNPVTLGTSASISCRSSK |
| | | FTSYWMSWVKQRPEQGLEWIGRIDPYDS | SLLHSYGITYLYWYLQKPGQSPQLLIY |
| | | ETHYNQKFKDKAILTVDKSSSTAYMQLS | QMSNLASGVPDRFSSSGSGTDFTLRIS |
| | | SLTSEDSAVYYCARTYYGNYDAMDYWGQ | RVEAEDVGVYYCAQNLELPLTFGAGTK |
| | | GTSVTVSS [SEQ ID NO: 15] | LELK [SEQ ID NO: 16] |
| 20027 | 3D7 | QVQLQQPGAELVRPGASVKLSCKASGYT | QIVLTQSPAIMSASPGEKVTMTCSASS |
| | | FTSYWINWVKQRPGQGLEWIGNIYPSDN | SVSYMHWYQQKSGTSPKRWVYDTSKLA |
| | | YTNYNQKFKDKATLTVDKSSSTAYMQLS | SGVPARFSGSGSGTSYSLTISSMEAED |
| | | SPTSEDSAVYYCTRGGGIYYENYFDYWG | VATYYCOQWSSNPPTFGGGTKLEIK |
| | | QGTTLTVSS [SEQ ID NO: 17] | [SEQ ID NO: 18] |
| 20028 | 3H1 | QVHLQQSGSELRIPGSSVKLSCKDFDSE | DVVMTQTPLSLPVSLGDQASISCRSSQ |
| | | VFPIAYMSWVRQKPGHGFEWIGDILPSI | SLVHSNGNTYLHWYLQKPGQSPKLLIY |
| | | GRTIYGEKFEDKATLDADTVSNTAYLDL | KVSNRFFGVPDRFSGSGSGTDFTLKIS |
| | | NSLTSEDSAIYYCSRGDYYYGSREYAMD | RVEAEDLGVYFCSQTTYVPLTFGAGTK |
| | | YWGQGTSVTVSS [SEQ ID NO: 19] | LELK [SEQ ID NO: 20] |
| 20029 | 3E7 | QVQLQESGGGLVKPGGSRKLSCAASGFT | NIVMTQSPKSMSMSVGERVTLSCKASE |
| | | FSEFGMHWVRQAPDKGLEWVAYISSGSS | NVGSYVSWYQQKPEKSPKLLIYGASNR |
| | | TIYYADTVKGRFTISRDNAKNTLFLQMT | YTGVPDRFTGSGSATDFTLTISSVQAE |
| | | SLRSEDTAMYYCARDWVDYWGQGTALTV | DLADYHCGQSYSYPLTFGAGTKLELK |
| | | SS [SEQ ID NO: 21] | [SEQ ID NO: 10] |
| 20030 | 3G4 | QVQLKQSGPGLVQPSQSLSITCTVSGFS | DIVMTQSPSSLAVTAGEKVTMRCKSSQ |
| | | LTSYGVHWVRQSPGKGLEWLGVIWSGGS | SLLMSVNQNNYLSWYQQKQGQPPKLLI |
| | | TDYNGAFISRLSISKDNSKSQVFFKMNS | YGASIRESWVPDRFTGSGSGTDFTLTI |
| | | LQADDTAMYFCARDRGGGFDYWGQGTTL | SNVHAEDLAVYYCQHNHGSFLPYTFGG |
| | | TVSS [SEQ ID NO: 23] | GTKLEIK [SEQ ID NO: 24] |
| 20031 | 4B11 | QVQLQQSGPELVKPGASVKISCKASGYV | DVVMTQTPLSLPVSLGDQASISCRSSQ |
| | | FSSYWNNWVKQRPGKGLEWIGRIYPGNG | SLVHSNGNTYLHWYLQKPGQSPKLLIY |
| | | DTNYNGKFKDKATLTADKFSSTAYMQLS | KVSNRFFGVPDRFSGSGSGTDFTLKIS |
| | | NLTSEDSAVYFCASYYELDYWGQGTSVT | RVEAEDLGVYFCSQTTYVPLTFGAGTK |
| | | VSS [SEQ ID NO: 25] | LELK [SEQ ID NO: 26] |
| 20032 | 4E6 | QVQLKESGPGLVAPSQSLSITCTVSGFS | DIQMTQTTSSLSASLGDRVTFSCSASQ |
| | | LTSYAINWVRQPPGKGLEWLGVLWPGGG | GISNYLNWYQQKPDGTVKLLIYYTSSL |
| | | TNYNSALKSRLSISKDNSKSQVFLKMNS | HLGVPSRFSGSGSGTDYSLTISNLEPE |
| | | LQTDDTARYYCARGSGTWYFDVWGAGTT | DIATYYCQQYSKLPWTFGGGTKLEIK |
| | | VTVSS [SEQ ID NO: 27] | [SEQ ID NO: 28] |
| 20033 | 4F9 | QVQLQQPGAELVKPGASVMLSCKASGYT | NIVMTQSPKSMSMSVGERVTLSCKASD |
| | | FTSYWMHWVKQRPGQGLEWIGEINLRNG | NVGISVSWYQQKPEQSPKLLIYGASNR |
| | | GTNYYEKFKTRATLTVDKSSSTAYMQLS | YTGVPDRFTGTGSATDFTLTISSVQAE |
| | | SLTSEDSAVYYCTILTSAPSYWGQGTLV | DLADYHCGQSYSYPFTFGSGTKLEIK |
| | | TVSA [SEQ ID NO: 29] | [SEQ ID NO: 30] |
| 20034 | 4G10 | QLQESGAELLRPGASVKLSCTASGFNIK | DIVMTQAAFSNPVTLGTSASISCRSNK |
| | | DDYLHWVKQRPEQGLEWIGWIDPENGDT | SLLHSDGITYLFWYLQRPGQSPQLLIY |
| | | EYASKFQGKATIKADTSSNTAYLQLSSL | RMSNLASGVPDRFSGSGSGTDFTLRIS |
| | | TSEDTAVYYCSTQGFACWGQGTLVTVSA | RVEAEDVGVYYCAQMVEFPRTFGGGTK |
| | | [SEQ ID NO: 31] | LEIK [SEQ ID NO: 32] |
| 20035 | 5E2 | EVKLVESEGGLVQPGSSMKLSCTASGFT | DVLMTQTPLSLPVSLGDQASISCRSSQ |
| | | FSDYYMAWVRQVPEKGLEWVAHINYDGS | SIVHSNGNTYLEWYLQKPGQSPKLLIY |
| | | GTYYLDSLKGRFIISRDNAKNILYLQMS | KVSNRFSGVPDRFSGSGSGTDFTLKIS |
| | | SLKSEDTATYYCARDCYGSSSYAVDYWG | RVEAEDLGVYYOFQGSHVPWTFGGGTK |
| | | QGTSVTVSS [SEQ ID NO: 33] | LEIK [SEQ ID NO: 34] |
| 20036 | 5G8 | QVQLQESGPGLVQPTQSLSITCTVSGFS | DIVMTQSQKFMSTSVGDRVSVTCKASQ |
| | | LISYGVHWVRQSPGKGLEWLGVIWSGGS | NVGTNVAWYQQKPGQSPKALIYSASYR |
| | | TDYNAAFISRLSISKDNSKSQVFFKMNS | DSGVPDRFTGSGSGTDFTLTISNVQSE |
| | | LQADDTAIYYCARNPLTATVMDYWGQGT | DLAEYLCQQYNSYPLTFGAGTKLELK |
| | | SVTVSS [SEQ ID NO: 35] | [SEQ ID NO: 36] |
| 20037 | 6B3 | QVQLKESGPGLVAPSQSLSITCTVSGFS | DIQMTQTTSSLSASLGDRVTISCSASQ |
| | | LTSYAISWVRQPPGKGLEWLGVIWPGGG | GISNYLNWYQQKPDGTVKLLIYYTSSL |
| | | TNYNSALKSRLSISKDNSKSQVFLKMNS | HSGVPSRFSGSGSGTDYSLTISNLEPE |
| | | LQTDDTARYYCARGTGTWYFDVWGAGTT | DIATYYCQQYSDLPWTFGGGTKLEIK |
| | | VTVSS [SEQ ID NO: 37] | [SEQ ID NO: 38] |

TABLE 14
Amino acid and DNA sequences of heavy and light chain humanized 1C8,
1G1 and 5G8

| | | |
|---|---|---|
| SEQ | | |
| ID | | |
| NO | DESCRIPTION | SEQUENCE |

Protein Sequences

| | | |
|---|---|---|
| 45 | Humanized 1C8 heavy chain | EVQLVESGGGLIQPGGSLRLSCAASGFSLTTYAISWV |
| | variable domain sequence 1 | RQAPGKGLEWVSIIWPGGGTNYADSVKGRFTISRDNS |
| | (H5): Heavy chain mouse 1C8 | KNTLYLQMNSLRAEDTAVYYCARGAGTWYFDVWG |
| | CDRs ported onto framework | QGTLVTVSS |
| | of germline IGHV3-66*03 | |
| 51 | Humanized 1C8 heavy chain | EVQLVESGGGLIQPGGSLRLSCAVSGFSLTTYAISWV |
| | variable domain sequence 2 | RQAPGKGLEWLGIIWPGGGTNYADSVKGRFTISRDNS |
| | (H6) | KNTLYLQMNSLRAEDTAVYYCARGAGTWYFDVWG |
| | | QGTLVTVSS |
| 52 | Humanized 1C8 heavy chain | EVQLVESGGGLIQPGGSLRLSCAVSGFSLTTYAISWV |
| | variable domain sequence 3 | RQAPGKGLEWLGIIWPGGGTNYADSVKGRFTISKDNS |
| | (H7) | KNTLYLQMNSLRAEDTAVYYCARGAGTWYFDVWG |
| | | QGTLVTVSS |
| 53 | Humanized 1C8 heavy chain | EVQLVESGGGLIQPGGSLRISCAVSGFSLTTYAISWVR |
| | variable domain sequence 4 | QAPGKGLEWLGIIWPGGGTNYADSLKGRLTISKDNSK |
| | (H8) | NTVLQMNSLRTEDTAVYYCARGAGTWYFDVWGQ |
| | | GTLVTVSS |
| 48 | Humanized 1C8 light chain | DIQMTQSPSSLSASVGDRVTITCSASQGISNYLNWVQ |
| | variable domain sequence 1 | QKPGKAPKLLIYYTSSLHSGVPSRFSGSGSGTDFTFTIS |
| | (L1): Light chain mouse 1C8 | SLQPEDIATYYCQQYSKFPWTGQGTKVEIK |
| | CDRs ported onto framework | |
| | of germline IGKV1D-33*01 | |
| 54 | Humanized 1C8 light chain | DIQMTQSPSSLSASVGDRVTITCSASQGISNYLNWYQ |
| | variable domain sequence 2 | QKPGKARKLLIYYTSSLHSGVPSRFSGSGSGTDYTLTI |
| | (L2) | SSLQPEDIATYYCQQYSKFPWTFGQGTKLEIK |
| 55 | Humanized 1C8 light chain | DIQMTQSPSSLSASVGDRVTITCSASQGISNYLNWYQ |
| | variable domain sequence 3 | QKPGKTVKLLIYYTSSLHSGVPSRFSGSGSGTDYTLTI |
| | (L3) | SSLQPEDIATYYCQQYSKFPWTFGQGTKLEIK |
| 46 | Humanized 1G1 heavy chain | EVQLVESGGGLVQPGGSLRLSCAASGFTFSEFGMHW |
| | variable domain sequence 1 | VRQAPGKGLEWVSYISSGGSTIYYADSVKGRFTISRD |
| | (H1): Heavy chain mouse 1G1 | NAKNSLYLQMNSLRAEDTAVYYCARDWVDYWGQG |
| | CDRs ported onto framework: | TLVTVSS |
| | of germline IGHV3-48*03 | |
| 56 | Humanized 1G1 heavy chain | EVQLVESGGGLVQPGGSLRLSCAASGFTFSEFGMHW |
| | variable domain sequence 2 | VRQAPGKGLEWVYAYISSGGSTIYYADSVKGRFTISRD |
| | (H2) | NAKNSLYLQMNSLRAEDTAVYYCARDWVDYWGQG |
| | | TLLTVSS |
| 57 | Humanized 1G1 heavy chain | EVQLVESGGGLVQPGGSLRLSCAASGFTFSEFGMHW |
| | variable domain sequence 3 | VRQAPGKGLEWVAYISSGGSTIYYADSVKGRFTISRD |
| | (H3) | NAKNSLYLQMNSLRAEDTAMYYCARDWVDYWGQG |
| | | TLLTVSS |
| 49 | Humanized 1G1 light chain | EIVLTQSPATLSLSPGERATLSCKASENVGSYVSWYQ |
| | variable domain sequence 1 | QKPGQAPRLLIYGASNRYTGIPARFSGSGSGTDFTLTI |
| | (L1): Light chain mouse 1G1 | SSLEPEDFAVYYCGQSYSYPLTFGGGTKVEIK |
| | CDRs ported onto framework | |
| | of germline IGKV3-11*01 | |
| 58 | Humanized 1G1 light chain | EIVLTQSPATLSLSPGERATLSCKASENVGSYVSWYQ |
| | variable domain sequence 2 | QKPGQAPRLLIYGASNRYTGVPARFSGSGSGTDFTLTI |
| | (L2) | SSLEPEDFAVYYCGQSYSYPLTFGGGTKVEIK |
| 59 | Humanized 1G1 light chain | EIVMTQSPATLSLSPGERVTLSCKASENVGSYVSWYQ |
| | variable domain sequence 3 | QKPGQAPRLLIYGASNRYTGVPARFSGSGSGTDFTLTI |
| | (L3) | SSVEPEDFAVYYCGQSYSYPLTFGGGTKLELK |
| 60 | Humanized 1G1 light chain | EIVMTQSPATLSLSPGERVTLSCKASENVGSYVSWYQ |
| | variable domain sequence 4 | QKPGQAPRLLIYGASNRYTGVPARFSGSGSGTDFTLTI |
| | (L4) | SSVEPEDLAVYYCGQSYSYPLTEGGGTKLELK |
| 47 | Humanized 5G8 heavy chain | QVQLQESGPGLVKPSETLSLTCTVSGFSLISYGVHWIR |
| | variable domain sequence 1 | QPPGKGLEWIGVIWSGGSTDYNPSLKSRVTISVDTSK |
| | (H1): Heavy chain mouse 5G8 | NQFSLKLSSVTAADTAVYYCARNPLTATVMDYWGQ |
| | CDRs ported onto framework | GTLVTVSS |
| | of germline 1GHV4-59*08 | |
| 61 | Humanized 5G8 heavy chain | QVQLQESGPGLVKPSETLSLTCTVSGFSLISYGVHWV |
| | variable domain sequence 2 | RQPPGKGLEWLGVIWSGGSTDYNPSLKSRVTISKDTS |
| | (H2) | KNQFSLKLSSVTAADTAVYYCARNPLTATVMDYWG |
| | | QGTLVTVSS |
| 62 | Humanized 5G8 heavy chain | QVQLQESGPGLVKPSETLSITCTVSGFSLISYGVHWVR |
| | variable domain sequence 3 | QPPGKGLEWLGVIWSGGSTDYNPSLKSRVTISKDTSK |
| | (H3) | NQVSFKLSSLTAADTAIYYCARNPLTATVMDYWGQG |
| | | TLVTVSS |
| 63 | Humanized 5G8 heavy chain | QVQLQESGPGLVKPSETLSITCTVSGFSLISYGVHWVR |
| | variable domain sequence 4 | QPPGKGLEWLGVIWSGGSTDYNPSLKSRVTISKDNSK |
| | (H4) | NQVSFKLSSLTAADTAIYYCARNPLTATVMDYWGQG |
| | | TLVTVSS |
| 50 | Humanized 5G8 light chain | DIQMTQSPSSLSASVGDRVTITCRASQNVGTNVAWFQ |
| | variable domain sequence 1 | QKPGKAPKSLIYSASYRDSGVPSRFSGSGSGTDFTLTI |
| | (L1): Light chain mouse 5G8 | SSLQPEDFATYYCQQYNSYPLTFGGGTKVEIK |
| | CDRs ported onto framework | |
| | of germline IGKV1-16*01 | |
| 64 | Humanized 5G8 light chain | DIQMTQSPSSLSASVGDRVTITCRASQNVGTNVAWY |
| | variable domain sequence 2 | QQKPGKAPKALIYSASYRDSGVPSRFSGSGSGTDFTL |
| | (L2) | TISSLQPEDFATYYCQQYNSYPLTFGGGTKVEIK |
| 65 | Humanized 5G8 light chain | DIQMTQSPSSLSASVGDRVTVTCRASQNVGTNVAWY |
| | variable domain sequence 3 | QQKPGKAPKALIYSASYRDSGVPSRFSGSGSGTDFTL |
| | (L3) | TISSVQPEDFATYYCQQYNSYPLTFGGGTKLEIK |
| 66 | Humanized 5G8 light chain | DIQMTQSPSSLSASVGDRVTVTCRASQNVGTNVAWY |
| | variable domain sequence 4 | QQKPGKAPKALIYSASYRDSGVPSRFSGSGSGTDFTL |
| | (L4) | TISSVQPEDLATYYCQQYNSYPLTFGGGTKLEIK |
| 67 | Ckappa domain of IGKC*01 | RTVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSFNRGEC |
| 68 | IgG1 CH1-hinge-CH2-CH3 | ASTKGPSVFPLAPSSKSTSGGTAALGCLVKDYFPEPVT |
| | | VSWNSGALTSGVHTFPAVLQSSGLYSLSSVVTVPSSS |
| | | LGTQTYICNVNHKPSNTKVDKKVEPKSCDKTHTCPPC |
| | | PAPEAAGGPSVFLFPPKPKDTLMISRTPEVTCVVVSVS |
| | | HEDPEVKFNWYVDGVEVHNAKTKPREEQYNSTYRV |
| | | VSVLTVLHQDWLNGKEYKCKVSNKALPAPIEKTISK |
| | | AKGQPREPQVYTLPPSRDELTKNQVSLTCLVKGFYPS |
| | | DIAVEWESNGQPENNYKTTPPVLDSDGSFFLYSKLTV |
| | | DKSRWQQGNVFSCSVMEALHNHYTQKSLSLSPG |
| 69 | IgG1 Fc sequence 231-446 | APEAAGGPSVFLFPPKPKDTLMISRTPEVTCVVVSVSH |
| | (EU-numbering), without | EDPEVKFNWYVDGVEVHNAKTKPREEQYNSTYRVV |
| | hinge | SVLTVLHQDWLNGKEYKCKVSNKALPAPIEKTISKA |
| | | KGQPREPQVYTLPPSRDELTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPG |

TABLE 15
Sequences used in the construction of 4-1BB × HER2 antibodies

| SEQ ID NO: | Paratope/domain | Target | Sequence |
|---|---|---|---|
| 71 | MOR7480.1 VH | 4-1BB | EVQLVQSGAEVKKPGESLRISCKGSGYSFSTYWI |
| | | | SWVRQMPGKGLEWMGKIYPGDSYTNYSPSFQG |
| | | | QVTISADKSISTAYLQWSSLKASDTAMYYCARG |
| | | | YGIFDYWGQGTLVTVSS |
| 72 | MOR7480.1 VL | 4-1BB | SYELTQPPSVSVSPGQTASITCSGDNIGDQYAHW |
| | | | YQQKPGQSPVLVIYQDKNRPSGIPERFSGSNSGN |
| | | | TATLTISGTQAMDEADYYCATYTGFGSLAVFGG |
| | | | GTKLTVL |
| 73 | Anti-HER2 scFv | HER2 | DIQMTQSPSSLSASVGDRVTITCRASQDVNTAVA |
| | | | WYQQKPGKAPKLLIYSASFLYSGVPSRFSGSRSG |
| | | | TDFTLTISSLQPEDFATYYCQQHYTTPPTFGQGT |
| | | | KVEIKGGSGGGSGGGSGGGSGGGSGEVQLVESG |
| | | | GGLVQPGGSLRLSCAASGFNIKDTYIHWVRQAP |
| | | | GKGLEWVARIYPTNGYTRYADSVKGRFTISADT |
| | | | SKNTAYLQMNSLRAEDTAVYYCSRWGGDGFYA |
| | | | MDYWGQGTLVTVSS |

TABLE 16
Sequences used in the preparation of additional 4-1BB × TAA
bispecific antibody constructs

| | Paratope | SEQ ID | |
|---|---|---|---|
| Target | Source | NO: | scFv Sequence |
| Mesothelin | RG7787 | 74 | QVQLVQSGAEVKKPGASVKVSCKASGYSFTGYTMNWVR |
| | | | QAPGQGLEWMGLITPYNGASSYNQKFRGKATMTVDTST |
| | | | STVYMELSSLRSEDTAVYYCARGGYDGRGFDYWGQGTL |
| | | | VTVSSGGGGSGGGGSGGGGSDIQMTQSPSSLSASVGDR |
| | | | VTITCSASSSVSYMHWYQQKSGKAPKLLIYDTSKLASG |
| | | | VPSRFSGSGSGTDFTLTISSLQPEDFATYYCQQWSKHP |
| | | | LTFGQGTKLEIK |
| Mesothelin | Anetumab | 75 | QVELVQSGAEVKKPGESLKISCKGSGYSFTSYWIGWVR |
| | | | QAPGKGLEWMGIIDPGDSRTRYSPSFQGQVTISADKSI |
| | | | STAYLQWSSLKASDTAMYYCARGQLYGGTYMDGWGQGT |
| | | | LVTVSSVEGGSGGSGGSGGSGGVDDIALTQPASVSGSP |
| | | | GQSITISCTGTSSDIGGYNSVSWYQQHPGKAPKLMIYG |
| | | | VNNRPSGVSNRFSGSKSGNTASLTISGLQAEDEADYYC |
| | | | SSYDIESATPVFGGGTKLTVL |
| NaPi2b | Lifastuzumab | 76 | EVQLVESGGGLVQPGGSLRLSCAASGFSFSDFAMSWVR |
| | | | QAPGKGLEWVATIGRVAFHTYYPDSMKGRFTISRDNSK |
| | | | NTLYLQMNSLRAEDTAVYYCARHRGFDVGHFDFWGQGT |
| | | | LVTVSSGGGGSGGGGSGGGGSDIQMTQSPSSLSASVGD |
| | | | RVTITCRSSETLVHSSGNTYLEWYQQKPGKAPKLLIYR |
| | | | VSNRFSGVPSRFSGSGSGTDFTLTISSLQPEDFATYYC |
| | | | FQGSFNPLTFGQGTKVEIK |
| NaPi2b | MX-35 | 77 | DIQMTQSPSSLSASVGDRVTITCSASQDIGNFLNWYQQ |
| | | | KPGKTVKVLIYYTSSLYSGVPSRFSGSGSGTDYTLTIS |
| | | | SLQPEDFATYYCQQYSKLPLTFGQGTKLELKGGGGSGG |
| | | | GGSGGGGSQVQLVQSGAEVVKPGASVKMSCKASGYTFT |
| | | | GYNIHWVKQAPGQGLEWIGAIYPGNGDTSYKQKFRGRA |
| | | | TLTADTSTSTVYMELSSLRSEDSAVYYCARGETARATF |
| | | | AYWGQGTLVTSS |
| FRα | Mirvetuximab | 78 | QVQLVQSGAEVVKPGASVKISCKASGYTFTGYFMNWVK |
| | | | QSPGQSLEWIGRIHPYDGDTFYNQKFQGKATLTVDKSS |
| | | | NTAHMELLSLTSEDFAVYYCTRYDGSRAMDYWGQGTTV |
| | | | TVSSGGGGSGGGGSGGGGSDIVLTQSPLSLAVSLGQPA |
| | | | IISCKASQSVSFAGTSLMHWYHQKPGQQPRLLIYRASN |
| | | | LEAGVPDRFSGSGSKTDFTLTISPVEAEDAATYYCQQS |
| | | | REYPYTFGGGTKLEIK |

TABLE 17
VH and VL sequences used in the preparation of constructs

| Name | Domain | Sequence |
|---|---|---|
| HER2 scFv having | VH | EVQLVESGGGLVQPGGSLRLSCAASGFNIKDTYIHWVRQAPGKGLEW |
| SEQ ID NO 73 | | VARIYPTNGYTRYADSVKGRFTISADTSKNTAYLQMNSLRAEDTAVY |
| | | YCSRWGGDGFYAMDYWGQGTLVTVSS (SEQ ID NO: 322) |
| | VL | DIQMTQSPSSLSASVGDRVTITCRASQDVNTAVAWYQQKPGKAPKLLI |
| | | YSASFLYSGVPSRFSGSRSGTDFTLTISSLQPEDFATYYCQQHYTTPPTF |
| | | GQGTKVEIK (SEQ ID NO: 323) |
| MSLN scFv having | VH | QVQLVQSGAEVKKPGASVKVSCKASGYSFTGYTMNWVRQAPGQGLE |
| SEQ ID NO: 74 | | WMGLITPYNGASSYNQKFRGKATMTVDTSTSTVYMELSSLRSEDTAV |
| (RG7787) | | YYCARGGYDGRGFDYWGQGTLVTVSS (SEQ ID NO: 324) |
| | VL | DIQMTQSPSSLSASVGDRVTITCSASSSVSYMHWYQQKSGKAPKLLIY |
| | | DTSKLASGVPSRFSGSGSGTDFTLTISSLQPEDFATYYCQQWSKHPLTF |
| | | GQGTKLEIK (SEQ ID NO: 325) |
| MSLN scFv having | VH | QVELVQSGAEVKKPGESLKISCKGSGYSFTSYWIGWVRQAPGKGLEW |
| SEQ ID NO: 75 | | MGIIDPGDSRTRYSPSFQGQVTISADKSISTAYLQWSSLKASDTAMYY |
| (anetumab) | | CARGQLYGGTYMDGWGQGTLVTVSS (SEQ ID NO: 326) |
| | VL | DIALTQPASVSGSPGQSITISCTGTSSDIGGYNSVSWYQQHPGKAPKLM |
| | | IYGVNNRPSGVSNRFSGSKSGNTASLTISGLQAEDEADYYCSSYDIESA |
| | | TPVFGGGTKLTVL (SEQ ID NO: 327) |
| NaPi2b scFv | VH | EVQLVESGGGLVQPGGSLRLSCAASGFSFSDFAMSWVRQAPGKGLEW |
| having SEQ ID | | VATIGRVAFHTYYPDSMKGRFTISRDNSKNTLYLQMNSLRAEDTAVY |
| NO: 76 | | YCARHRGFDVGHFDFWGQGTLVTVSS (SEQ ID NO: 328) |
| (lifastuzumab) | VL | DIQMTQSPSSLSASVGDRVTITCRSSETLVHSSGNTYLEWYQQKPGKA |
| | | PKLLIYRVSNRFSGVPSRFSGSGSGTDFTLTISSLQPEDFATYYCFQGSF |
| | | NPLTFGQGTKVEIK (SEQ ID NO: 329) |
| NaPi2b scFv | VH | QVQLVQSGAEVVKPGASVKMSCKASGYTFTGYNIHWVKQAPGQGLE |
| having SEQ ID | | WIGAIYPGNGDTSYKQKFRGRATLTADTSTSTVYMELSSLRSEDSAVY |
| NO: 77 (MX-35) | | YCARGETARATFAYWGQGTLVTVSS (SEQ ID NO: 330) |
| | VL | DIQMTQSPSSLSASVGDRVTITCSASQDIGNFLNWYQQKPGKTVKVLI |
| | | YYTSSLYSGVPSRFSGSGSGTDYTLTISSLQPEDFATYYCQQYSKLPLT |
| | | FGQGTKLELK (SEQ ID NO: 331) |
| FRα scFv having | VH | QVQLVQSGAEVVKPGASVKISCKASGYTFTGYFMNWVKQSPGQSLE |
| SEQ ID NO: 78 | | WIGRIHPYDGDTFYNQKFQGKATLTVDKSSNTAHMELLSLTSEDFAV |
| (mirvetuximab) | | YYCTRYDGSRAMDYWGQGTTVTVSS (SEQ ID NO: 332) |
| | VL | DIVLTQSPLSLAVSLGQPAIISCKASQSVSFAGTSLMHWYHQKPGQQP |
| | | RLLIYRASNLEAGVPDRFSGSGSKTDFTLTISPVEAEDAATYYCQQSRE |
| | | YPYTFGGGTKLEIK (SEQ ID NO: 333) |
| 8K22 | VH | QEQLVESGGGLVQPEGSLTLTCKASGFTISNNYYMCWVRQAPGKGLE |
| | | WIACIYGGISGRTYYASWAKGRFTISKTSSTTVTLQMTSLTAADTATY |
| | | FCVRGYVGTSNLWGPGTLVTVSS (SEQ ID NO: 298) |
| 8K22 | VL | DIVMTQTPASVEAAVGGTVTIKCQASQSIYSSLAWYQQKPGQSPKLLI |
| | | YDASHLASGVPSRFSGSRYGTEFTLTISGVQCDDAATYYCQGGWYSS |
| | | AATYVPNTFGGGTEVVVK (SEQ ID NO: 299) |
| 1H06 | VH | QEQLVESGGGLVQPEGSLTLTCTASKFSFSSLYYMCWVRQAPGKGLE |
| | | WIACVYGGSSGNTYYASWAKGRFTISKASSTTVTLQLTSLTAADTAT |
| | | YFCARFDVDGSGFNLWGPGTLVTVSS (SEQ ID NO: 300) |
| 1H06 | VL | DIVMTQTPSSVSAAVGGTVTIKCQASQTIGSSLAWYQQKPGQPPKLLI |
| | | YRASTLASGVSSRFRGSGSGTEYTLTISDLECADAATYYCQWTDYGYI |
| | | YIWAFGGGTEVVVK (SEQ ID NO: 301) |
| Pertuzumab | VH | EVQLVESGGGLVQPGGSLRLSCAASGFTFTDYTMDWVRQAPGKGLE |
| | | WVADVNPNSGGSIYNQRFKGRFTLSVDRSKNTLYLQMNSLRAEDTAV |
| | | YYCARNLGPSFYFDYWGQGTLVTVSS (SEQ ID NO: 302) |
| Pertuzumab | VL | DIQMTQSPSSLSASVGDRVTITCKASQDVSIGVAWYQQKPGKAPKLLI |
| | | YSASYRYTGVPSRFSGSGSGTDFTLTISSLQPEDFATYYCQQYYIYPYT |
| | | FGQGTKVEIK (SEQ ID NO: 303) |
| Farletuzumab | VH | EVQLVESGGGVVQPGRSLRLSCSASGFTFSGYGLSWVRQAPGKGLEW |
| | | VAMISSGGSYTYYADSVKGRFAISRDNAKNTLFLQMDSLRPEDTGVY |
| | | FCARHGDDPAWFAYWGQGTPVTVSS (SEQ ID NO: 304) |
| Farletuzumab | VL | DIQLTQSPSSLSASVGDRVTITCSVSSSISSNNLHWYQQKPGKAPKPWI |
| | | YGTSNLASGVPSRFSGSGSGTDYTFTISSLQPEDIATYYCQQWSSYPYM |
| | | YTFGQGTKVEIK (SEQ ID NO: 305) |

TABLE 18
CDR sequences

| SEQ NAME | Definition | HCDR1 | HCDR2 | HCDR3 | LCDR1 | LCDR2 | LCDR3 |
|---|---|---|---|---|---|---|---|
| 1H06_VH | Kabat | SLYYMC (SEQ ID | CVYGGSSGNTYYASWAKG | FDVDGSGFNL (SEQ ID | | | |
| | | NO: 470) | (SEQ ID NO: 471) | NO: 472) | | | |
| | Chothia | KFSFSSLY (SEQ | YGGSSGN (SEQ ID NO: 474) | FDVDGSGFNL (SEQ ID | | | |
| | | ID NO: 473) | | NO: 472) | | | |
| | IMGT | KFSFSSLYY (SEQ | VYGGSSGNT (SEQ ID NO: | ARFDVDGSGFNL (SEQ | | | |
| | | ID NO: 475) | 476) | ID NO: 477) | | | |
| | AbM | KFSFSSLYYMC | CVYGGSSGNTY (SEQ ID | FDVDGSGFNL (SEQ ID | | | |
| | | (SEQ ID NO: 478) | NO: 479) | NO: 472) | | | |
| | Contact | SSLYYMC (SEQ | WIACVYGGSSGNTY (SEQ | ARFDVDGSGFN (SEQ ID | | | |
| | | ID NO: 480) | ID NO: 481) | NO: 482) | | | |
| 1H06_VL | Kabat | | | | QASQTIGSSLA (SEQ ID | RASTLAS (SEQ | QWTDYGYIYIWA |
| | | | | | NO: 483) | ID NO: 484) | (SEQ ID NO: 485) |
| | Chothia | | | | QASQTIGSSLA (SEQ ID | RASTLAS (SEQ | QWTDYGYIYIWA |
| | | | | | NO: 483) | ID NO: 484) | (SEQ ID NO: 485) |
| | IMGT | | | | QTIGSS (SEQ ID NO: | RAS | QWTDYGYIYIWA |
| | | | | | 486) | | (SEQ ID NO: 485) |
| | AbM | | | | QASQTIGSSLA (SEQ ID | RASTLAS (SEQ | QWTDYGYIYIWA |
| | | | | | NO: 483) | ID NO: 484) | (SEQ ID NO: 485) |
| | Contact | | | | GSSLAWY (SEQ ID NO: | LLIYRASTLA | QWTDYGYIYIW (SEQ |
| | | | | | 487) | (SEQ ID NO: | ID NO: 489) |
| | | | | | | 488) | |
| 8K22_VH | Kabat | NNYYMC (SEQ ID | CIYGGISGRTYYASWAKG | GYVGTSNL (SEQ ID NO: | | | |
| | | NO: 490) | (SEQ ID NO: 491) | 492) | | | |
| | Chothia | GFTISNNY (SEQ | YGGISGR (SEQ ID NO: | GYVGTSNL (SEQ ID NO: | | | |
| | | ID NO: 493) | 494) | 492) | | | |
| | IMGT | GFTISNNYY (SEQ | IYGGISGRT (SEQ ID NO: | VRGYVGTSNL (SEQ ID | | | |
| | | ID NO: 495) | 496) | NO: 497) | | | |
| | AbM | GFTISNNYYMC | CIYGGISGRTY (SEQ ID NO: | GYVGTSNL (SEQ ID NO: | | | |
| | | (SEQ ID NO: 498) | 499) | 492) | | | |
| | Contact | SNNYYMC (SEQ | WIACIYGGISGRTY (SEQ ID | VRGYVGTSN (SEQ ID | | | |
| | | ID NO: 500) | NO: 501) | NO: 502) | | | |
| 8K22_VL | Kabat | | | | QASQSIYSSLA (SEQ ID | DASHLAS | QGGWYSSAATYVPNT |
| | | | | | NO: 503) | (SEQ ID NO: | (SEQ ID NO: 505) |
| | | | | | | 504) | |
| | Chothia | | | | QASQSIYSSLA (SEQ ID | DASHLAS | QGGWYSSAATYVPNT |
| | | | | | NO: 503) | (SEQ ID NO: | (SEQ ID NO: 505) |
| | | | | | | 504) | |
| | IMGT | | | | QSIYSS (SEQ ID NO: 506) | DAS | QGGWYSSAATYVPNT |
| | | | | | | | (SEQ ID NO: 505) |
| | AbM | | | | QASQSIYSSLA (SEQ ID | DASHLAS | QGGWYSSAATYVPNT |
| | | | | | NO: 503) | (SEQ ID NO: | (SEQ ID NO: 505) |
| | | | | | | 504) | |
| | Contact | | | | YSSLAWY (SEQ ID NO: | LLIYDASHLA | QGGWYSSAATYVPN |
| | | | | | 507) | (SEQ ID NO: | (SEQ ID NO: 509) |
| | | | | | | 508) | |
| Mouse 1G1 | Kabat | | | | KASENVGSYVS (SEQ ID | GASNRYT | GQSYSYPLT (SEQ ID |
| VL or mouse | | | | | NO: 510) | (SEQ ID NO: | NO: 512) |
| 3E7 VL | | | | | | 511) | |
| corresponding | Chothia | | | | KASENVGSYVS (SEQ ID | GASNRYT | GQSYSYPLT (SEQ ID |
| to | | | | | NO: 510) | (SEQ ID NO: | NO: 512) |
| SEQ ID | | | | | | 511) | |
| NO: 10 | IMGT | | | | ENVGSY (SEQ ID NO: | GAS | GQSYSYPLT (SEQ ID |
| | | | | | 513) | | NO: 512) |
| | AbM | | | | KASENVGSYVS (SEQ ID | GASNRYT | GQSYSYPLT (SEQ ID |
| | | | | | NO: 510) | (SEQ ID NO: | NO: 512) |
| | | | | | | 511) | |
| | Contact | | | | GSYVSWY (SEQ ID NO: | LLIYGASNRY | GQSYSYPL (SEQ ID |
| | | | | | 514) | (SEQ ID NO: | NO: 516) |
| | | | | | | 515) | |
| Mouse 2A7 | Kabat | SYVMH (SEQ ID | YINPYNDGTKYNEKFKG | LGSRGTWFAY (SEQ ID | | | |
| VH | | NO: 517) | (SEQ ID NO: 518) | NO: 519) | | | |
| corresponding | Chothia | GYTFTSY (SEQ ID | NPYNDG (SEQ ID NO: 521) | LGSRGTWFAY (SEQ ID | | | |
| to | | NO: 520) | | NO: 519) | | | |
| SEQ ID | IMGT | GYTFTSYV (SEQ | INPYNDGT (SEQ ID NO: | ARLGSRGTWFAY (SEQ | | | |
| NO: 11 | | ID NO: 522) | 523) | ID NO: 524) | | | |
| | Abm | GYTFTSYVMH | YINPYNDGTK (SEQ ID NO: | LGSRGTWFAY (SEQ ID | | | |
| | | (SEQ ID NO: 525) | 526) | NO: 519) | | | |
| | Contact | TSYVMH (SEQ ID | WIGYINPYNDGTK (SEQ ID | ARLGSRGTWFA (SEQ | | | |
| | | NO: 527) | NO: 528) | ID NO: 529) | | | |
| Mouse 2A7 | Kabat | | | | KASQDVGTAVG (SEQ | WASTRRT | QQYSSYPLT (SEQ ID |
| VL | | | | | ID NO: 530) | (SEQ ID NO: | NO: 532) |
| corresponding | | | | | | 531) | |
| to | Chothia | | | | KASQDVGTAVG (SEQ | WASTRRT | QQYSSYPLT (SEQ ID |
| SEQ ID | | | | | ID NO: 530) | (SEQ ID NO: | NO: 532) |
| NO: 12 | | | | | | 531) | |
| | IMGT | | | | QDVGTA (SEQ ID NO: | WAS | QQYSSYPLT (SEQ ID |
| | | | | | 533) | | NO: 532) |
| | AbM | | | | KASQDVGTAVG (SEQ | WASTRRT | QQYSSYPLT (SEQ ID |
| | | | | | ID NO: 530) | (SEQ ID NO: | NO: 532) |
| | | | | | | 531) | |
| | Contact | | | | GTAVGWY (SEQ ID NO: | LLIYWASTRR | QQYSSYPL (SEQ ID |
| | | | | | 534) | (SEQ ID NO: | NO: 536) |
| | | | | | | 535) | |
| Mouse 2E8 | Kabat | SYGVH (SEQ ID | VIWSGGSTDYNAAFIS | NPLTATVMDY (SEQ ID | | | |
| VH | | NO: 537) | (SEQ ID NO: 538) | NO: 539) | | | |
| corresponding | Chothia | GFSLTSY (SEQ ID | WSGGS (SEQ ID NO: 541) | NPLTATVMDY (SEQ ID | | | |
| to | | NO: 540) | | NO: 539) | | | |
| SEQ ID | IMGT | GFSLTSYG (SEQ | IWSGGST (SEQ ID NO: 543) | ARNPLTATVMDY (SEQ | | | |
| NO: 13 | | ID NO: 542) | | ID NO: 544) | | | |
| | AbM | GFSLTSYGVH | VIWSGGSTD (SEQ ID NO: | NPLTATVMDY (SEQ ID | | | |
| | | (SEQ ID NO: 545) | 546) | NO: 539) | | | |
| | Contact | TSYGVH (SEQ ID | WLGVIWSGGSTD (SEQ ID | ARNPLTATVMD (SEQ | | | |
| | | NO: 547) | NO: 548) | ID NO: 549) | | | |
| Mouse 2E8 | Kabat | | | | KASQNVGTNVA (SEQ | SASYRHS (SEQ | QQYNSYPLT (SEQ ID |
| VL | | | | | ID NO: 550) | ID NO: 551) | NO: 552) |
| corresponding | Chothia | | | | KASQNVGTNVA (SEQ | SASYRHS (SEQ | QQYNSYPLT (SEQ ID |
| to | | | | | ID NO: 550) | ID NO: 551) | NO: 552) |
| SEQ ID | IMGT | | | | QNVGTN (SEQ ID NO: | SAS | QQYNSYPLT (SEQ ID |
| NO: 14 | | | | | 553) | | NO: 552) |
| | AbM | | | | KASQNVGTNVA (SEQ | SASYRHS (SEQ | QQYNSYPLT (SEQ ID |
| | | | | | ID NO: 550) | ID NO: 551) | NO: 552) |
| | Contact | | | | GTNVAWY (SEQ ID NO: | VLIYSASYRH | QQYNSYPL (SEQ ID |
| | | | | | 554) | (SEQ ID NO: | NO: 556) |
| | | | | | | 555) | |
| Mouse 2H9 | Kabat | SYWMS (SEQ ID | RIDPYDSETHYNQKFKD | TYYGNYDAMDY (SEQ | | | |
| VH | | NO: 557) | (SEQ ID NO: 558) | ID NO: 559) | | | |
| corresponding | Chothia | GYPFTSY (SEQ ID | DPYDSE (SEQ ID NO: 561) | TYYGNYDAMDY (SEQ | | | |
| to | | NO: 560) | | ID NO: 559) | | | |
| SEQ ID | IMGT | GYPFTSYW (SEQ | IDPYDSET (SEQ ID NO: | ARTYYGNYDAMDY | | | |
| NO: 15 | | ID NO: 562) | 563) | (SEQ ID NO: 564) | | | |
| | AbM | GYPFTSYWMS | RIDPYDSETH (SEQ ID NO: | TYYGNYDAMDY (SEQ | | | |
| | | (SEQ ID NO: 565) | 566) | ID NO: 559) | | | |
| | Contact | TSYWMS (SEQ ID | WIGRIDPYDSETH (SEQ ID | ARTYYGNYDAMD (SEQ | | | |
| | | NO: 567) | NO: 568) | ID NO: 569) | | | |
| Mouse 2H9 | Kabat | | | | RSSKSLLHSYGITYLY | QMSNLAS | AQNLELPLT (SEQ ID |
| VL | | | | | (SEQ ID NO: 570) | (SEQ ID NO: | NO: 572) |
| corresponding | | | | | | 571) | |
| to | Chothia | | | | RSSKSLLHSYGITYLY | QMSNLAS | AQNLELPLT (SEQ ID |
| SEQ ID | | | | | (SEQ ID NO: 570) | (SEQ ID NO: | NO: 572) |
| NO: 16 | | | | | | 571) | |
| | IMGT | | | | KSLLHSYGITY (SEQ ID | QMS | AQNLELPLT (SEQ ID |
| | | | | | NO: 573) | | NO: 572) |
| | AbM | | | | RSSKSLLHSYGITYLY | QMSNLAS | AQNLELPLT (SEQ ID |
| | | | | | (SEQ ID NO: 570) | (SEQ ID NO: | NO: 572) |
| | | | | | | 571) | |
| | Contact | | | | LHSYGITYLYWY (SEQ | LLIYQMSNLA | AQNLELPL (SEQ ID |
| | | | | | ID NO: 574) | (SEQ ID NO: | NO: 576) |
| | | | | | | 575) | |
| Mouse 3D7 | Kabat | SYWIN (SEQ ID | NIYPSDNYTNYNQKFKD | GGGIYYENYFDY (SEQ | | | |
| VH | | NO: 577) | (SEQ ID NO: 578) | ID NO: 579) | | | |
| corresponding | Chothia | GYTFTSY (SEQ ID | YPSDNY (SEQ ID NO: 580) | GGGIYYENYFDY (SEQ | | | |
| to | | NO: 520) | | ID NO: 579) | | | |
| SEQ ID | IMGT | GYTFTSYW (SEQ | IYPSDNYT (SEQ ID NO: | TRGGGIYYENYFDY | | | |
| NO: 17 | | ID NO: 581) | 582) | (SEQ ID NO: 583) | | | |
| | Abm | GYTFTSYWIN | NIYPSDNYTN (SEQ ID NO: | GGGIYYENYFDY (SEQ | | | |
| | | (SEQ ID NO: 584) | 585) | ID NO: 579) | | | |
| | Contact | TSYWIN (SEQ ID | WIGNIYPSDNYTN (SEQ ID | TRGGGIYYENYFD (SEQ | | | |
| | | NO: 586) | NO: 587) | ID NO: 588) | | | |
| Mouse 3D7 | Kabat | | | | SASSSVSYMH (SEQ ID | DTSKLAS (SEQ | QQWSSNPPT (SEQ ID |
| VL | | | | | NO: 589) | ID NO: 590) | NO: 591) |
| corresponding | Chothia | | | | SASSSVSYMH (SEQ ID | DTSKLAS (SEQ | QQWSSNPPT (SEQ ID |
| to | | | | | NO: 589) | ID NO: 590) | NO: 591) |
| SEQ ID | IMGT | | | | SSVSY (SEQ ID NO: 592) | DTS | QQWSSNPPT (SEQ ID |
| NO: 18 | | | | | | | NO: 591) |
| | AbM | | | | SASSSVSYMH (SEQ ID | DTSKLAS (SEQ | QQWSSNTPT (SEQ ID |
| | | | | | NO: 589) | ID NO: 590) | NO: 591) |
| | Contact | | | | SYMHWY (SEQ ID NO: | RWVYDTSKL | QQWSSNPP (SEQ ID |
| | | | | | 593) | A (SEQ ID NO: | NO: 595) |
| | | | | | | 594) | |
| Mouse 3H1 | Kabat | PIAYMS (SEQ ID | DILPSIGRTIYGEKFED | GDYYYGSREYAMDY | | | |
| VH | | NO: 596) | (SEQ ID NO: 597) | (SEQ ID NO: 598) | | | |
| corresponding | Chothia | DSEVFPIA (SEQ | LPSIGR (SEQ ID NO: 600) | GDYYYGSREYAMDY | | | |
| to | | ID NO: 599) | | (SEQ ID NO: 598) | | | |
| SEQ ID | IMGT | DSEVFPIAY (SEQ | ILPSIGRT (SEQ ID NO: | SRGDYYYGSREYAMDY | | | |
| NO: 19 | | ID NO: 601) | 602) | (SEQ ID NO: 603) | | | |
| | AbM | DSEVFPIAYMS | DILPSIGRTI (SEQ ID NO: | GDYYYGSREYAMDY | | | |
| | | (SEQ ID NO: 604) | 605) | (SEQ ID NO: 598) | | | |
| | Contact | FPIAYMS (SEQ ID | WIGDILPSIGRTI (SEQ ID | SRGDYYYGSREYAMD | | | |
| | | NO: 606) | NO: 607) | (SEQ ID NO: 608) | | | |
| Mouse 3H1 | Kabat | | | | RSSQSLVHSNGNTYLH | KVSNRFF (SEQ | SQTTYVPLT (SEQ ID |
| VL | | | | | (SEQ ID NO: 609) | ID NO: 610) | NO: 611) |
| corresponding | Chothia | | | | RSSQSLVHSNGNTYLH | KVSNRFF (SEQ | SQTTYVPLT (SEQ ID |
| to | | | | | (SEQ ID NO: 609) | ID NO: 610) | NO: 611) |
| SEQ ID | IMGT | | | | QSLVHSNGNTY (SEQ ID | KVS | SQTTYVPLT (SEQ ID |
| NO: 20 | | | | | NO: 612) | | NO: 611) |
| | AbM | | | | RSSQSLVHSNGNTYLH | KVSNRFF (SEQ | SQTTYVPLT (SEQ ID |
| | | | | | (SEQ ID NO: 609) | ID NO: 610) | NO: 611) |
| | Contact | | | | VHSNGNTYLHWY (SEQ | LLIYKVSNRF | SQTTYVPL (SEQ ID |
| | | | | | ID NO: 613) | (SEQ ID NO: | NO: 615) |
| | | | | | | 614) | |
| Mouse 3E7 | Kabat | EFGMH (SEQ ID | YISSGSSTIYYADTVKG | DWVDY (SEQ ID NO: | | | |
| VH | | NO: 616) | (SEQ ID NO: 617) | 618) | | | |
| corresponding | Chothia | GFTFSEF (SEQ ID | SSGSST (SEQ ID NO: 620) | DWVDY (SEQ ID NO: | | | |
| to | | NO: 619) | | 618) | | | |
| SEQ ID | IMGT | GFTFSEFG (SEQ | ISSGSSTI (SEQ ID NO: | ARDWVDY (SEQ ID NO: | | | |
| NO: 21 | | ID NO: 621) | 622) | 623) | | | |
| | AbM | GFTFSEFGMH | YISSGSSTIY (SEQ ID NO: | DWVDY (SEQ ID NO: | | | |
| | | (SEQ ID NO: 624) | 625) | 618) | | | |
| | Contact | SEFGMH (SEQ ID | WVAYISSGSSTIY (SEQ ID | ARDWVD (SEQ ID NO: | | | |
| | | NO: 626) | NO: 627) | 628) | | | |
| Mouse 3G4 | Kabat | SYGVH (SEQ ID | VIWSGGSTDYNGAFIS | DRGGGFDY (SEQ ID | | | |
| VH | | NO: 537) | (SEQ ID NO: 629) | NO: 630) | | | |
| corresponding | Chothia | GFSLTSY (SEQ ID | WSGGS (SEQ ID NO: 541) | DRGGGFDY (SEQ ID | | | |
| to | | NO: 540) | | NO: 630) | | | |
| SEQ ID | IMGT | GFSLTSYG (SEQ | IWSGGST (SEQ ID NO: 543) | ARDPGGGFDY (SEQ ID | | | |
| NO: 23 | | ID NO: 542) | | NO: 631) | | | |
| | AbM | GFSLTSYGVH | VIWSGGSTD (SEQ ID NO: | DRGGGFDY (SEQ ID | | | |
| | | (SEQ ID NO: 545) | 546) | NO: 630) | | | |
| | Contact | TSYGVH (SEQ ID | WLGVIWSGGSTD (SEQ ID | ARDRGGGFD (SEQ ID | | | |
| | | NO: 547) | NO: 548) | NO: 632) | | | |
| Mouse 3G4 | Kabat | | | | KSSQSLLWSVNQNNYL | GASIRES (SEQ | QHNHGSFLPYT (SEQ |
| VL | | | | | S (SEQ ID NO: 633) | ID NO: 634) | ID NO: 635) |
| corresponding | Chothia | | | | KSSQSLLWSVNQNNYL | GASIRES (SEQ | QHNHGSFLPYT (SEQ |
| to | | | | | S (SEQ ID NO: 633) | ID NO: 634) | ID NO: 635) |
| SEQ ID | IMGT | | | | QSLLWSVNQNNY (SEQ | GAS | QHNHGSFLPYT (SEQ |
| NO: 24 | | | | | ID NO: 636) | | ID NO: 635) |
| SEQ ID | AbM | | | | KSSQSLLWSVNQNNYL | GASIRES (SEQ | QHNHGSFLPYT (SEQ |
| NO: 24 | | | | | S (SEQ ID NO: 633) | ID NO: 634) | ID NO: 635) |
| | Contact | | | | LWSVNQNNYLSWY | LLIYGASIRE | QHNHGSFLPY (SEQ |
| | | | | | (SEQ ID NO: 637) | (SEQ ID NO: | ID NO: 639) |
| | | | | | | 638) | |
| Mouse 4B11 | Kabat | SYWMN (SEQ ID | RIYPGNGDTNYNGKFKD | YYELDY (SEQ ID NO: | | | |
| VH | | NO: 640) | (SEQ ID NO: 641) | 642) | | | |
| corresponding | Chothia | GYVFSSY (SEQ ID | YPGNGD (SEQ ID NO: 644) | YYELDY (SEQ ID NO: | | | |
| to | | NO: 643) | | 642) | | | |
| SEQ ID | IMGT | GYVFSSYW (SEQ | IYPGNGDT (SEQ ID NO: | ASYYELDY (SEQ ID | | | |
| NO: 25 | | ID NO: 645) | 646) | NO: 647) | | | |
| | AbM | GYVFSSYWMN | RIYPGNGDTN (SEQ ID NO: | YYELDY (SEQ ID NO: | | | |
| | | (SEQ ID NO: 648) | 649) | 642) | | | |
| | Contact | SSYWMN (SEQ ID | WIGRIYPGNGDTN (SEQ ID | ASYYELD (SEQ ID NO: | | | |
| | | NO: 650) | NO: 651) | 652) | | | |
| Mouse 4B11 | Kabat | | | | RSSQSLVHSNGNTYLH | KVSNRFF (SEQ | SQTTYVPLT (SEQ ID |
| VL | | | | | (SEQ ID NO: 609) | ID NO: 610) | NO: 611) |
| corresponding | Chothia | | | | RSSQSLVHSNGNTYLH | KVSNRFF (SEQ | SQTTYVPLT (SEQ ID |
| to | | | | | (SEQ ID NO: 609) | ID NO: 610) | NO: 611) |
| SEQ ID | IMGT | | | | QSLVHSNGNTY (SEQ ID | KVS | SQTTYVPLT (SEQ ID |
| NO: 26 | | | | | NO: 612) | | NO: 611) |
| | AbM | | | | RSSQSLVHSNGNTYLH | KVSNRFF (SEQ | SQTTYVPLT (SEQ ID |
| | | | | | (SEQ ID NO: 609) | ID NO: 610) | NO: 611) |
| | Contact | | | | VHSNGNTYLHWY (SEQ | LLIYKVSNRF | SQTTYVPL (SEQ ID |
| | | | | | ID NO: 613) | (SEQ ID NO: | NO: 615) |
| | | | | | | 614) | |
| Mouse 4E6 | Kabat | SYAIN (SEQ ID | VLWPGGGTNYNSALKS | GSGTWYFDV (SEQ ID | | | |
| VH | | NO: 653) | (SEQ ID NO: 654) | NO: 655) | | | |
| corresponding | Chothia | GFSLTSY (SEQ ID | WPGGG (SEQ ID NO: 656) | GSGTWYFDV (SEQ ID | | | |
| to | | NO: 540) | | NO: 655) | | | |
| SEQ ID | IMGT | GFSLTSYA (SEQ | LWPGGGT (SEQ ID NO: | ARGSGTWYFDV (SEQ | | | |
| NO: 27 | | ID NO: 657) | 658) | ID NO: 659) | | | |
| | AbM | GFSLTSYAIN | VLWPGGGTN (SEQ ID NO: | GSGTWYFDV (SEQ ID | | | |
| | | (SEQ ID NO: 660) | 661) | NO: 655) | | | |
| | Contact | TSYAIN (SEQ ID | WLGVLWPGGGTN (SEQ ID | ARGSGTWYFD (SEQ ID | | | |
| | | NO: 662) | NO: 663) | NO: 664) | | | |
| Mouse 4E6 | Kabat | | | | SASQGISNYLN (SEQ ID | YTSSLHL (SEQ | QQYSKLPWT (SEQ ID |
| VL | | | | | NO: 665) | ID NO: 666) | NO: 667) |
| corresponding | Chothia | | | | SASQGISNYLN (SEQ ID | YTSSLHL (SEQ | QQYSKLPWT (SEQ ID |
| to | | | | | NO: 665) | ID NO: 666) | NO: 667) |
| SEQ ID | IMGT | | | | QGISNY (SEQ ID NO: | YTS | QQYSKLPWT (SEQ ID |
| NO: 28 | | | | | 668) | | NO: 667) |
| | AbM | | | | SASQGISNYLN (SEQ ID | YTSSLHL (SEQ | QQYSKLPWT (SEQ ID |
| | | | | | NO: 665) | ID NO: 666) | NO: 667) |
| | Contact | | | | SNYLNWY (SEQ ID NO: | LLIYYTSSLH | QQYSKLPW (SEQ ID |
| | | | | | 669) | (SEQ ID NO: | NO: 671) |
| | | | | | | 670) | |
| Mouse 4F9 | Kabat | SYWMH (SEQ ID | EINLRNGGTNYYEKFKT | LTSAPSY (SEQ ID NO: | | | |
| VH | | NO: 672) | (SEQ ID NO: 673) | 674) | | | |
| corresponding | Chothia | GYTFTSY (SEQ ID | NLRNGG (SEQ ID NO: 675) | LTSAPSY (SEQ ID NO: | | | |
| to | | NO: 520) | | 674) | | | |
| SEQ ID | IMGT | GYTFTSYW (SEQ | INLRNGGT (SEQ ID NO: | TILTSAPSY (SEQ ID NO: | | | |
| NO: 29 | | ID NO: 581) | 676) | 677) | | | |
| | AbM | GYTFTSYWMH | EINLRNGGTN (SEQ ID NO: | LTSAPSY (SEQ ID NO: | | | |
| | | (SEQ ID NO: 678) | 679) | 674) | | | |
| | Contact | TSYWMH (SEQ ID | WIGEINLRNGGTN (SEQ ID | TILTSAPS (SEQ ID NO: | | | |
| | | NO: 680) | NO: 681) | 682) | | | |
| Mouse 1B2 | Kabat | SFWIN (SEQ ID | NIYPDSSSTNYNEKFKN | SLTFDY (SEQ ID NO: | | | |
| VH | | NO: 683) | (SEQ ID NO: 684) | 685) | | | |
| corresponding | Chothia | GYTFTSF (SEQ ID | YPDSSS (SEQ ID NO: 687) | SLTFDY (SEQ ID NO: | | | |
| to | | NO: 686) | | 685) | | | |
| SEQ ID NO: 3 | IMGT | GYTFTSFW (SEQ | IYPDSSST (SEQ ID NO: | ARSLTFDY (SEQ ID NO: | | | |
| | | ID NO: 688) | 689) | 690) | | | |
| | AbM | GYIFTSFWIN | NIYPDSSSTN (SEQ ID NO: | SLIFDY (SEQ ID NO: | | | |
| | | (SEQ ID NO: 691) | 692) | 685) | | | |
| | Contact | TSFWIN (SEQ ID | WIGNIYPDSSSTN (SEQ ID | ARSLTFD (SEQ ID NO: | | | |
| | | NO: 693) | NO: 694) | 695) | | | |
| Mouse 4F9 | Kabat | | | | KASDNVGISVS (SEQ ID | GASNRYT | GQSYSYPFT (SEQ ID |
| VL | | | | | NO: 696) | (SEQ ID NO: | NO: 697) |
| corresponding | | | | | | 511) | |
| to | Chothia | | | | KASDNVGISVS (SEQ ID | GASNRYT | GQSYSYPFT (SEQ ID |
| SEQ ID | | | | | NO: 696) | (SEQ ID NO: | NO: 697) |
| NO: 30 | | | | | | 511) | |
| | IMGT | | | | DNVGIS (SEQ ID NO: | GAS | GQSYSYPFT (SEQ ID |
| | | | | | 698) | | NO: 697) |
| | AbM | | | | KASDNVGISVS (SEQ ID | GASNRYT | GQSYSYPFT (SEQ ID |
| | | | | | NO: 696) | (SEQ ID NO: | NO: 697) |
| | | | | | | 511) | |
| | Contact | | | | GISVSWY (SEQ ID NO: | LLIYGASNRY | GQSYSYPF (SEQ ID |
| | | | | | 699) | (SEQ ID NO: | NO: 700) |
| | | | | | | 515) | |
| Mouse 4G10 | Kabat | DDYLH (SEQ ID | WIDPENGDTEYASKFQG | QGFAC (SEQ ID NO: | | | |
| VH | | NO: 701) | (SEQ ID NO: 702) | 703) | | | |
| corresponding | Chothia | GFNIKDD (SEQ ID | DPENGD (SEQ ID NO: 705) | QGFAC (SEQ ID NO: | | | |
| to | | NO: 704) | | 703) | | | |
| SEQ ID | IMGT | GFNIKDDY (SEQ | IDPENGDT (SEQ ID NO: | STQGFAC (SEQ ID NO: | | | |
| NO: 31 | | ID NO: 706) | 707) | 708) | | | |
| | AbM | GFNIKDDYLH | WIDPENGDTE (SEQ ID NO: | QGFAC (SEQ ID NO: | | | |
| | | (SEQ ID NO: 709) | 710) | 703) | | | |
| | Contact | KDDYLH (SEQ ID | WIGWIDPENGDTE (SEQ ID | STQGFA (SEQ ID NO: | | | |
| | | NO: 711) | NO: 712) | 713) | | | |
| Mouse 4G10 | Kabat | | | | RSNKSLLHSDGITYLF | RMSNLAS | AQMVEFPRT (SEQ ID |
| VL | | | | | (SEQ ID NO: 714) | (SEQ ID NO: | NO: 716) |
| corresponding | | | | | | 715) | |
| to | Chothia | | | | RSNKSLLHSDGITYLF | RMSNLAS | AQMVEFPRT (SEQ ID |
| SEQ ID | | | | | (SEQ ID NO: 714) | (SEQ ID NO: | NO: 716) |
| NO: 32 | | | | | | 715) | |
| | IMGT | | | | KSLLHSDGITY (SEQ ID | RMS | AQMVEFPRT (SEQ ID |
| | | | | | NO: 717) | | NO: 716) |
| | AbM | | | | RSNKSLLHSDGITYLF | RMSNLAS | AQMVEFPRT (SEQ ID |
| | | | | | (SEQ ID NO: 714) | (SEQ ID NO: | NO: 716) |
| | | | | | | 715) | |
| | Contact | | | | LHSDGITYLFWY (SEQ | LLIYRMSNLA | AQMVEFPR (SEQ ID |
| | | | | | ID NO: 718) | (SEQ ID NO: | NO: 720) |
| | | | | | | 719) | |
| Mouse 5E2 | Kabat | DYYMA (SEQ ID | HINYDSGTYYLDSLKG | DCYGSSSYAVDY (SEQ | | | |
| VH | | NO: 721) | (SEQ ID NO: 722) | ID NO: 723) | | | |
| corresponding | Chothia | GFTFSDY (SEQ ID | NYDGSG (SEQ ID NO: 725) | DCYGSSSYAVDY (SEQ | | | |
| to | | NO: 724) | | ID NO: 723) | | | |
| SEQ ID | IMGT | GFTFSDYY (SEQ | INYDGSGT (SEQ ID NO: | ARDCYGSSSYAVDY | | | |
| NO: 33 | | ID NO: 726) | 727) | (SEQ ID NO: 728) | | | |
| | AbM | GFTFSDYYMA | HINYDGSGTY (SEQ ID NO: | DCYGSSSYAVDY (SEQ | | | |
| | | (SEQ ID NO: 729) | 730) | ID NO: 723) | | | |
| | Contact | SDYYMA (SEQ ID | WVAHINYDGSGTY (SEQ | ARDCYGSSSYAVD | | | |
| | | NO: 731) | ID NO: 732) | (SEQ ID NO: 733) | | | |
| Mouse 5E2 | Kabat | | | | RSSQSIVHSNGNTYLE | KVSNRFS (SEQ | FQGSHVPWT (SEQ ID |
| VL | | | | | (SEQ ID NO: 734) | ID NO: 735) | NO: 736) |
| corresponding | Chothia | | | | RSSQSIVHSNGNTYLE | KVSNRFS (SEQ | FQGSHVPWT (SEQ ID |
| to | | | | | (SEQ ID NO: 734) | ID NO: 735) | NO: 736) |
| SEQ ID | IMGT | | | | QSIVHSNGNTY (SEQ ID | KVS | FQGSHVPWT (SEQ ID |
| NO: 34 | | | | | NO: 737) | | NO: 736) |
| | AbM | | | | RSSQSIVHSNGNTYLE | KVSNRFS (SEQ | FQGSHVPWT (SEQ ID |
| | | | | | (SEQ ID NO: 734) | ID NO: 735) | NO: 736) |
| | Contact | | | | VHSNGNTYLEWY (SEQ | LLIYKVSNRF | FQGSHVPW (SEQ ID |
| | | | | | ID NO: 738) | (SEQ ID NO: | NO: 739) |
| | | | | | | 614) | |
| Mouse 5G8 | Kabat | SYGVH (SEQ ID | VIWSGGSTDYNAAFIS | NPLTATVMDY (SEQ ID | | | |
| VH | | NO: 537) | (SEQ ID NO: 538) | NO: 539) | | | |
| corresponding | Chothia | GFSLISY (SEQ ID | WSGGS (SEQ ID NO: 541) | NPLTATVMDY (SEQ ID | | | |
| to | | NO: 740) | | NO: 539) | | | |
| SEQ ID | IMGT | GFSLISYG (SEQ | IWSGGST (SEQ ID NO: 543) | ARNPLTATVMDY (SEQ | | | |
| NO: 35 | | ID NO: 741) | | ID NO: 544) | | | |
| | AbM | GFSLISYGVH | VIWSGGSTD (SEQ ID NO: | NPLTATVMDY (SEQ ID | | | |
| | | (SEQ ID NO: 742) | 546) | NO: 539) | | | |
| | Contact | ISYGVH (SEQ ID | WLGVIWSGGSTD (SEQ ID | ARNPLTATVMD (SEQ | | | |
| | | NO: 743) | NO: 548) | ID NO: 549) | | | |
| Mouse 5G8 | Kabat | | | | KASQNVGTNVA (SEQ | SASYRDS (SEQ | QQYNSYPLT (SEQ ID |
| VL | | | | | ID NO: 550) | ID NO: 744) | NO: 552) |
| corresponding | Chothia | | | | KASQNVGTNVA (SEQ | SASYRDS (SEQ | QQYNSYPLT (SEQ ID |
| to | | | | | ID NO: 550) | ID NO: 744) | NO: 552) |
| SEQ ID | IMGT | | | | QNVGTN (SEQ ID NO: | SAS | QQYNSYPLT (SEQ ID |
| NO: 36 | | | | | 553) | | NO: 552) |
| | AbM | | | | KASQNVGTNVA (SEQ | SASYRDS (SEQ | QQYNSYPLT (SEQ ID |
| | | | | | ID NO: 550) | ID NO: 744) | NO: 552) |
| | Contact | | | | GTNVAWY (SEQ ID NO: | ALIYSASYRD | QQYNSYPL (SEQ ID |
| | | | | | 554) | (SEQ ID NO: | NO: 556) |
| | | | | | | 745) | |
| Mouse 6B3 | Kabat | | | | SASQGISNYLN (SEQ ID | YTSSLHS (SEQ | QQYSDLPWT (SEQ ID |
| VL | | | | | NO: 665) | ID NO: 746) | NO: 747) |
| corresponding | Chothia | | | | SASQGISNYLN (SEQ ID | YTSSLHS (SEQ | QQYSDLPWT (SEQ ID |
| to | | | | | NO: 665) | ID NO: 746) | NO: 747) |
| SEQ ID | IMGT | | | | QGISNY (SEQ ID NO: | YTS | QQYSDLPWT (SEQ ID |
| NO: 38 | | | | | 668) | | NO: 747) |
| | AbM | | | | SASQGISNYLN (SEQ ID | YTSSLHS (SEQ | QQYSDLPWT (SEQ ID |
| | | | | | NO: 665) | ID NO: 746) | NO: 747) |
| | Contact | | | | SNYLNWY (SEQ ID NO: | LLIYYTSSLH | QQYSDLPW (SEQ ID |
| | | | | | 669) | (SEQ ID NO: | NO: 748) |
| | | | | | | 670) | |
| Mouse 1B2 | Kabat | | | | RSSQSIVHSNGNTYLE | KVSNRFS (SEQ | FQGSHVPYT (SEQ ID |
| VL | | | | | (SEQ ID NO: 734) | ID NO: 735) | NO: 749) |
| corresponding | Chothia | | | | RSSQSIVHSNGNTYLE | KVSNRFS (SEQ | FQGSHVPYT (SEQ ID |
| to | | | | | (SEQ ID NO: 734) | ID NO: 735) | NO: 749) |
| SEQ ID NO: 4 | IMGT | | | | QSIVHSNGNTY (SEQ ID | KVS | FQGSHVPYT (SEQ ID |
| | | | | | NO: 737) | | NO: 749) |
| | AbM | | | | RSSQSIVHSNGNTYLE | KVSNRFS (SEQ | FQGSHVPYT (SEQ ID |
| | | | | | (SEQ ID NO: 734) | ID NO: 735) | NO: 749) |
| | Contact | | | | VHSNGNTYLEWY (SEQ | LLIYKVSNRF | FQGSHVPY (SEQ ID |
| | | | | | ID NO: 738) | (SEQ ID NO: | NO: 750) |
| | | | | | | 614) | |
| Humanized | Kabat | TYAIS (SEQ ID | IIWPGGGTNYADSVKG | GAGTWYFDV (SEQ ID | | | |
| 1C8 VH H5 | | NO: 751) | (SEQ ID NO: 752) | NO: 753) | | | |
| corresponding | Chothia | GFSLTTY (SEQ ID | WPGGG (SEQ ID NO: 656) | GAGTWYFDV (SEQ ID | | | |
| to | | NO: 754) | | NO: 753) | | | |
| SEQ ID | IMGT | GFSLITYA (SEQ | IWPGGGT (SEQ ID NO: | ARGAGTWYFDV (SEQ | | | |
| NO: 45 | | ID NO: 755) | 756) | ID NO: 757) | | | |
| | AbM | GFSLTTYAIS | IIWPGGGTN (SEQ ID NO: | GAGTWYFDV (SEQ ID | | | |
| | | (SEQ ID NO: 758) | 759) | NO: 753) | | | |
| | Contact | TTYAIS (SEQ ID | WVSIIWPGGGTN (SEQ ID | ARGAGTWYFD (SEQ ID | | | |
| | | NO: 760) | NO: 761) | NO: 762) | | | |
| Humanized | Kabat | EFGMH (SEQ ID | YISSGGSTIYYADSVKG | DWVDY (SEQ ID NO: | | | |
| 1G1 VH H1 | | NO: 616) | (SEQ ID NO: 763) | 618) | | | |
| corresponding | Chothia | GFTFSEF (SEQ ID | SSGGST (SEQ ID NO: 764) | DWVDY (SEQ ID NO: | | | |
| to | | NO: 619) | | 618) | | | |
| SEQ ID | IMGT | GFTFSEFG (SEQ | ISSGGSTI (SEQ ID NO: | ARDWVDY (SEQ ID NO: | | | |
| NO: 46 | | ID NO: 621) | 765) | 623) | | | |
| | AbM | GFTFSEFGMH | YISSGGSTIY (SEQ ID NO: | DWVDY (SEQ ID NO: | | | |
| | | (SEQ ID NO: 624) | 766) | 618) | | | |
| | Contact | SEFGMH (SEQ ID | WVSYISSGGSTIY (SEQ ID | ARDWVD (SEQ ID NO: | | | |
| | | NO: 626) | NO: 767) | 628) | | | |
| Humanized | Kabat | SYGVH (SEQ ID | VIWSGGSTDYNPSLKS | NPLTATVMDY (SEQ ID | | | |
| 5G8 VH H1 | | NO: 537) | (SEQ ID NO: 768) | NO: 539) | | | |
| corresponding | Chothia | GFSLISY (SEQ ID | WSGGS (SEQ ID NO: 541) | NPLTATVMDY (SEQ ID | | | |
| to | | NO: 740) | | NO: 539) | | | |
| SEQ ID | IMGT | GFSLISYG (SEQ | IWSGGST (SEQ ID NO: | ARNPLTATVMDY (SEQ | | | |
| NO: 47 | | ID NO: 741) | 543) | ID NO: 544) | | | |
| | Abm | GFSLISYGVH | VIWSGGSTD (SEQ ID NO: | NPLTATVMDY (SEQ ID | | | |
| | | (SEQ ID NO: 742) | 546) | NO: 539) | | | |
| | Contact | ISYGVH (SEQ ID | WIGVIWSGGSTD (SEQ ID | ARNPLTATVMD (SEQ | | | |
| | | NO: 743) | NO: 769) | ID NO: 549) | | | |
| Humanized | Kabat | | | | SASQGISNYLN (SEQ ID | YTSSLHS (SEQ | QQYSKFPWT (SEQ ID |
| 1C8 VL L1 | | | | | NO: 665) | ID NO: 746) | NO: 770) |
| corresponding | Chothia | | | | SASQGISNYLN (SEQ ID | YTSSLHS (SEQ | QQYSKFPWT (SEQ ID |
| to | | | | | NO: 665) | ID NO: 746) | NO: 770) |
| SEQ ID | IMGT | | | | QGISNY (SEQ ID NO: | YTS | QQYSKFPWT (SEQ ID |
| NO: 48 | | | | | 668) | | NO: 770) |
| | AbM | | | | SASQGISNYLN (SEQ ID | YTSSLHS (SEQ | QQYSKFPWT (SEQ ID |
| | | | | | NO: 665) | ID NO: 746) | NO: 770) |
| | Contact | | | | SNYLNWY (SEQ ID NO: | LLIYYTSSLH | QQYSKFPW (SEQ ID |
| | | | | | 669) | (SEQ ID NO: | NO: 771) |
| | | | | | | 670) | |
| Humanized | Kabat | | | | KASENVGSYVS (SEQ ID | GASNRYT | GQSYSYPLT (SEQ ID |
| 1G1 VL L1 | | | | | NO: 510) | (SEQ ID NO: | NO: 512) |
| corresponding | | | | | | 511) | |
| to | Chothia | | | | KASENVGSYVS (SEQ ID | GASNRYT | GQSYSYPLT (SEQ ID |
| SEQ ID | | | | | NO: 510) | (SEQ ID NO: | NO: 512) |
| NO: 49 | | | | | | 511) | |
| | IMGT | | | | ENVGSY (SEQ ID NO: | GAS | GQSYSYPLT (SEQ ID |
| | | | | | 513) | | NO: 512) |
| | AbM | | | | KASENVGSYVS (SEQ ID | GASNRYT | GQSYSYPLT (SEQ ID |
| | | | | | NO: 510) | (SEQ ID NO: | NO: 512) |
| | | | | | | 511) | |
| | Contact | | | | GSYVSWY (SEQ ID NO: | LLIYGASNRY | GQSYSYPL (SEQ ID |
| | | | | | 514) | (SEQ ID NO: | NO: 516) |
| | | | | | | 515) | |
| Mouse 1C3 | Kabat | SYGVH (SEQ ID | VIWRGGSTDYNAAFIS | ENYDYDEFAY (SEQ ID | | | |
| VH | | NO: 537) | (SEQ ID NO: 772) | NO: 773) | | | |
| corresponding | Chothia | GFSLTSY (SEQ ID | WRGGS (SEQ ID NO: 774) | ENYDYDEFAY (SEQ ID | | | |
| to | | NO: 540) | | NO: 773) | | | |
| SEQ ID NO: 5 | IMGT | GFSLTSYG (SEQ | IWRGGST (SEQ ID NO: | ARENYDYDEFAY (SEQ | | | |
| | | ID NO: 542) | 775) | ID NO: 776) | | | |
| | AbM | GFSLTSYGVH | VIWRGGSTD (SEQ ID NO: | ENYDYDEFAY (SEQ ID | | | |
| | | (SEQ ID NO: 545) | 777) | NO: 773) | | | |
| | Contact | TSYGVH (SEQ ID | WLGVIWRGGSTD (SEQ ID | ARENYDYDEFA (SEQ | | | |
| | | NO: 547) | NO: 778) | ID NO: 779) | | | |
| Humanized | Kabat | | | | RASQNVGTNVA (SEQ | SASYRDS (SEQ | QQYNSYPLT (SEQ ID |
| 5G8 VL L1 | | | | | ID NO: 780) | ID NO: 744) | NO: 552) |
| corresponding | Chothia | | | | RASQNVGTNVA (SEQ | SASYRDS (SEQ | QQYNSYPLT (SEQ ID |
| to | | | | | ID NO: 780) | ID NO: 744) | NO: 552) |
| SEQ ID | IMGT | | | | QNVGTN (SEQ ID NO: | SAS | QQYNSYPLT (SEQ ID |
| NO: 50 | | | | | 553) | | NO: 552) |
| | AbM | | | | RASQNVGTNVA (SEQ | SASYRDS (SEQ | QQYNSYPLT (SEQ ID |
| | | | | | ID NO: 780) | ID NO: 744) | NO: 552) |
| | Contact | | | | GTNVAWF (SEQ ID NO: | SLIYSASYRD | QQYNSYPL (SEQ ID |
| | | | | | 781) | (SEQ ID NO: | NO: 556) |
| | | | | | | 782) | |
| Humanized | Kabat | TYAIS (SEQ ID | IIWPGGGTNYADSVKG | GAGTWYFDV (SEQ ID | | | |
| 1C8 VH H6 | | NO: 751) | (SEQ ID NO: 752) | NO: 753) | | | |
| corresponding | Chothia | GFSLTTY (SEQ ID | WPGGG (SEQ ID NO: 656) | GAGTWYFDV (SEQ ID | | | |
| to | | NO: 754) | | NO: 753) | | | |
| SEQ ID | IMGT | GFSLTTYA (SEQ | IWPGGGT (SEQ ID NO: | ARGAGTWYFDV (SEQ | | | |
| NO: 51 | | ID NO: 755) | 756) | ID NO: 757) | | | |
| | AbM | GFSLTTYAIS | IIWPGGGTN (SEQ ID NO: | GAGTWYFDV (SEQ ID | | | |
| | | (SEQ ID NO: 758) | 759) | NO: 753) | | | |
| | Contact | TTYAIS (SEQ ID | WLGIIWPGGGTN (SEQ ID | ARGAGTWYFD (SEQ ID | | | |
| | | NO: 760) | NO: 783) | NO: 762) | | | |
| Humanized | Kabat | TYAIS (SEQ ID | IIWPGGGTNYADSVKG | GAGTWYFDV (SEQ ID | | | |
| 1C8 VH H7 | | NO: 751) | (SEQ ID NO: 752) | NO: 753) | | | |
| corresponding | Chothia | GFSLTTY (SEQ ID | WPGGG (SEQ ID NO: 656) | GAGTWYFDV (SEQ ID | | | |
| to | | NO: 754) | | NO: 753) | | | |
| SEQ ID | IMGT | GFSLTTYA (SEQ | IWPGGGT (SEQ ID NO: | ARGAGTWYFDV (SEQ | | | |
| NO: 52 | | ID NO: 755) | 756) | ID NO: 757) | | | |
| | AbM | GFSLTTYAIS | IIWPGGGTN (SEQ ID NO: | GAGTWYFDV (SEQ ID | | | |
| | | (SEQ ID NO: 758) | 759) | NO: 753) | | | |
| | Contact | TTYAIS (SEQ ID | WLGIIWPGGGTN (SEQ ID | ARGAGTWYFD (SEQ ID | | | |
| | | NO: 760) | NO: 783) | NO: 762) | | | |
| Humanized | Kabat | TYAIS (SEQ ID | IIWPGGGTNYADSLKG | GAGTWYFDV (SEQ ID | | | |
| 1C8 VH H8 | | NO: 751) | (SEQ ID NO: 784) | NO: 753) | | | |
| corresponding | Chothia | GFSLTTY (SEQ ID | WPGGG (SEQ ID NO: 656) | GAGTWYFDV (SEQ ID | | | |
| to | | NO: 754) | | NO: 753) | | | |
| SEQ ID | IMGT | GFSLTTYA (SEQ | IWPGGGT (SEQ ID NO: | ARGAGTWYFDV (SEQ | | | |
| NO: 53 | | ID NO: 755) | 756) | ID NO: 757) | | | |
| | AbM | GFSLTTYAIS | IIWPGGGTN (SEQ ID NO: | GAGTWYFDV (SEQ ID | | | |
| | | (SEQ ID NO: 758) | 759) | NO: 753) | | | |
| | Contact | TTYAIS (SEQ ID | WLGIIWPGGGTN (SEQ ID | ARGAGTWYFD (SEQ ID | | | |
| | | NO: 760) | NO: 783) | NO: 762) | | | |
| Humanized | Kabat | | | | SASQGISNYLN (SEQ ID | YTSSLHS (SEQ | QQYSKFPWT (SEQ ID |
| 1C8 VL L2 | | | | | NO: 665) | ID NO: 746) | NO: 770) |
| corresponding | Chothia | | | | SASQGISNYLN (SEQ ID | YTSSLHS (SEQ | QQYSKFPWT (SEQ ID |
| to | | | | | NO: 665) | ID NO: 746) | NO: 770) |
| SEQ ID | IMGT | | | | QGISNY (SEQ ID NO: | YTS | QQYSKFPWT (SEQ ID |
| NO: 54 | | | | | 668) | | NO: 770) |
| | AbM | | | | SASQGISNYLN (SEQ ID | YTSSLHS (SEQ | QQYSKFPWT (SEQ ID |
| | | | | | NO: 665) | ID NO: 746) | NO: 770) |
| | Contact | | | | SNYLNWY (SEQ ID NO: | LLIYYTSSLH | QQYSKFPW (SEQ ID |
| | | | | | 669) | (SEQ ID NO: | NO: 771) |
| | | | | | | 670) | |
| Humanized | Kabat | | | | SASQGISNYLN (SEQ ID | YTSSLHS (SEQ | QQYSKFPWT (SEQ ID |
| 1C8 VL L3 | | | | | NO: 665) | ID NO: 746) | NO: 770) |
| corresponding | Chothia | | | | SASQGISNYLN (SEQ ID | YTSSLHS (SEQ | QQYSKFPWT (SEQ ID |
| to | | | | | NO: 665) | ID NO: 746) | NO: 770) |
| SEQ ID | IMGT | | | | QGISNY (SEQ ID NO: | YTS | QQYSKFPWT (SEQ ID |
| NO: 55 | | | | | 668) | | NO: 770) |
| | AbM | | | | SASQGISNYLN (SEQ ID | YTSSLHS (SEQ | QQYSKFPWT (SEQ ID |
| | | | | | 665) | ID NO: 746) | NO: 770) |
| | Contact | | | | SNYLNWY (SEQ ID NO: | LLIYYTSSLH | QQYSKFPW (SEQ ID |
| | | | | | 669) | (SEQ ID NO: | NO: 771) |
| | | | | | | 670) | |
| Humanized | Kabat | EFGMH (SEQ ID | YISSGGSTIYYADSVKG | DWVDY (SEQ ID NO: | | | |
| 1G1 VH H2 | | NO: 616) | (SEQ ID NO: 763) | 618) | | | |
| corresponding | Chothia | GFTFSEF (SEQ ID | SSGGST (SEQ ID NO: 764) | DWVDY (SEQ ID NO: | | | |
| to | | NO: 619) | | 618) | | | |
| SEQ ID | IMGT | GFTFSEFG (SEQ | ISSGGSTI (SEQ ID NO: | ARDWVDY (SEQ ID NO: | | | |
| NO: 56 | | ID NO: 621) | 765) | 623) | | | |
| | AbM | GFTFSEFGMH | YISSGGSTIY (SEQ ID NO: | DWVDY (SEQ ID NO: | | | |
| | | (SEQ ID NO: 624) | 766) | 618) | | | |
| | Contact | SEFGMH (SEQ ID | WVAYISSGGSTIY (SEQ ID | ARDWVD (SEQ ID NO: | | | |
| | | NO: 626) | NO: 785) | 628) | | | |
| Humanized | Kabat | EFGMH (SEQ ID | YISSGGSTIYYADSVKG | DWVDY (SEQ ID NO: | | | |
| 1G1 VH H3 | | NO: 616) | (SEQ ID NO: 763) | 618) | | | |
| corresponding | Chothia | GFTFSEF (SEQ ID | SSGGST (SEQ ID NO: 764) | DWVDY (SEQ ID NO: | | | |
| to | | NO: 619) | | 618) | | | |
| SEQ ID | IMGT | GFTFSEFG (SEQ | ISSGGSTI (SEQ ID NO: | ARDWVDY (SEQ ID NO: | | | |
| NO: 57 | | ID NO: 621) | 765) | 623) | | | |
| | AbM | GFTFSEFGMH | YISSGGSTIY (SEQ ID NO: | DWVDY (SEQ ID NO: | | | |
| | | (SEQ ID NO: 624) | 766) | 618) | | | |
| | Contact | SEFGMH (SEQ ID | WVAYISSGGSTIY (SEQ ID | ARDWVD (SEQ ID NO: | | | |
| | | NO: 626) | NO: 785) | 628) | | | |
| Humanized | Kabat | | | | KASENVGSYVS (SEQ ID | GASNRYT | GQSYSYPLT (SEQ ID |
| 1G1 VL L2 | | | | | NO: 510) | (SEQ ID NO: | NO: 512) |
| corresponding | | | | | | 511) | |
| to | Chothia | | | | KASENVGSYVS (SEQ ID | GASNRYT | GQSYSYPLT (SEQ ID |
| SEQ ID | | | | | NO: 510) | (SEQ ID NO: | NO: 512) |
| NO: 58 | | | | | | 511) | |
| | IMGT | | | | ENVGSY (SEQ ID NO: | GAS | GQSYSYPLT (SEQ ID |
| | | | | | 513) | | NO: 512) |
| | AbM | | | | KASENVGSYVS (SEQ ID | GASNRYT | GQSYSYPLT (SEQ ID |
| | | | | | NO: 510) | (SEQ ID NO: | NO: 512) |
| | | | | | | 511) | |
| | Contact | | | | GSYVSWY (SEQ ID NO: | LLIYGASNRY | GQSYSYPL (SEQ ID |
| | | | | | 514) | (SEQ ID NO: | NO: 516) |
| | | | | | | 515) | |
| Humanized | Kabat | | | | KASENVGSYVS (SEQ ID | GASNRYT | GQSYSYPLT (SEQ ID |
| 1G1 VL L3 | | | | | NO: 510) | (SEQ ID NO: | NO: 512) |
| corresponding | | | | | | 511) | |
| to | Chothia | | | | KASENVGSYVS (SEQ ID | GASNRYT | GQSYSYPLT (SEQ ID |
| SEQ ID | | | | | NO: 510) | (SEQ ID NO: | NO: 512) |
| NO: 59 | | | | | | 511) | |
| | IMGT | | | | ENVGSY (SEQ ID NO: | GAS | GQSYSYPLT (SEQ ID |
| | | | | | 513) | | NO: 512) |
| | AbM | | | | KASENVGSYVS (SEQ ID | GASNRYT | GQSYSYPLT (SEQ ID |
| | | | | | NO: 510) | (SEQ ID NO: | NO: 512) |
| | | | | | | 511) | |
| | Contact | | | | GSYVSWY (SEQ ID NO: | LLIYGASNRY | GQSYSYPL (SEQ ID |
| | | | | | 514) | (SEQ ID NO: | NO: 516) |
| | | | | | | 515) | |
| Mouse 1C3 | Kabat | | | | RSSQSIVHSNGNTYLE | KVSNRFS (SEQ | FQGSHVPYT (SEQ ID |
| VL | | | | | (SEQ ID NO: 734) | ID NO: 735) | NO: 749) |
| corresponding | Chothia | | | | RSSQSIVHSNGNTYLE | KVSNRFS (SEQ | FQGSHVPYT (SEQ ID |
| to | | | | | (SEQ ID NO: 734) | ID NO: 735) | NO: 749) |
| SEQ ID NO: 6 | IMGT | | | | QSIVHSNGNTY (SEQ ID | KVS | FQGSHVPYT (SEQ ID |
| | | | | | NO: 737) | | NO: 749) |
| | AbM | | | | RSSQSIVHSNGNTYLE | KVSNRFS (SEQ | FQGSHVPYT (SEQ ID |
| | | | | | (SEQ ID NO: 734) | ID NO: 735) | NO: 749) |
| | Contact | | | | VHSNGNTYLEWY (SEQ | LLIYKVSNRF | FQGSHVPY (SEQ ID |
| | | | | | ID NO: 738) | (SEQ ID NO: | NO: 750) |
| | | | | | | 614) | |
| Humanized | Kabat | | | | KASENVGSYVS (SEQ ID | GASNRYT | GQSYSYPLT (SEQ ID |
| 1G1 VL | | | | | NO: 510) | (SEQ ID NO: | NO: 512) |
| L4 | | | | | | 511) | |
| corresponding | Chothia | | | | KASENVGSYVS (SEQ ID | GASNRYT | GQSYSYPLT (SEQ ID |
| to | | | | | NO: 510) | (SEQ ID NO: | NO: 512) |
| SEQ ID | | | | | | 511) | |
| NO: 60 | IMGT | | | | ENVGSY (SEQ ID NO: | GAS | GQSYSYPLT (SEQ ID |
| | | | | | 513) | | NO: 512) |
| | AbM | | | | KASENVGSYVS (SEQ ID | GASNRYT | GQSYSYPLT (SEQ ID |
| | | | | | NO: 510) | (SEQ ID NO: | NO: 512) |
| | | | | | | 511) | |
| | Contact | | | | GSYVSWY (SEQ ID NO: | LLIYGASNRY | GQSYSYPL (SEQ ID |
| | | | | | 514) | (SEQ ID NO: | NO: 516) |
| | | | | | | 515) | |
| Humanized | Kabat | SYGVH (SEQ ID | VIWSGGSTDYNPSLKS | NPLTATVMDY (SEQ ID | | | |
| 5G8 VH H2 | | NO: 537) | (SEQ ID NO: 768) | NO: 539) | | | |
| corresponding | Chothia | GFSLISY (SEQ ID | WSGGS (SEQ ID NO: 541) | NPLTATVMDY (SEQ ID | | | |
| to | | NO: 740) | | NO: 539) | | | |
| SEQ ID | IMGT | GFSLISYG (SEQ | IWSGGST (SEQ ID NO: | ARNPLTATVMDY (SEQ | | | |
| NO: 61 | | ID NO: 741) | 543) | ID NO: 544) | | | |
| | AbM | GFSLISYGVH | VIWSGGSTD (SEQ ID NO: | NPLTATVMDY (SEQ ID | | | |
| | | (SEQ ID NO: 742) | 546) | NO: 539) | | | |
| | Contact | ISYGVH (SEQ ID | WLGVIWSGGSTD (SEQ ID | ARNPLTATVMD (SEQ | | | |
| | | NO: 743) | NO: 548) | ID NO: 549) | | | |
| Humanized | Kabat | SYGVH (SEQ ID | VIWSGGSTDYNPSLKS | NPLTATVMDY (SEQ ID | | | |
| 5G8 VH H3 | | NO: 537) | (SEQ ID NO: 768) | NO: 539) | | | |
| corresponding | Chothia | GFSLISY (SEQ ID | WSGGS (SEQ ID NO: 541) | NPLTATVMDY (SEQ ID | | | |
| to | | NO: 740) | | NO: 539) | | | |
| SEQ ID | IMGT | GFSLISYG (SEQ | IWSGGST (SEQ ID NO: | ARNPLTATVMDY (SEQ | | | |
| NO: 62 | | ID NO: 741) | 543) | ID NO: 544) | | | |
| | AbM | GFSLISYGVH | VIWSGGSTD (SEQ ID NO: | NPLTATVMDY (SEQ ID | | | |
| | | (SEQ ID NO: 742) | 546) | NO: 539) | | | |
| | Contact | ISYGVH (SEQ ID | WLGVIWSGGSTD (SEQ ID | ARNPLTATVMD (SEQ | | | |
| | | NO: 743) | NO: 548) | ID NO: 549) | | | |
| Humanized | Kabat | SYGVH (SEQ ID | VIWSGGSTDYNPSLKS | NPLTATVMDY (SEQ ID | | | |
| 5G8 VH H4 | | NO: 537) | (SEQ ID NO: 768) | NO: 539) | | | |
| corresponding | Chothia | GFSLISY (SEQ ID | WSGGS (SEQ ID NO: 541) | NPLTATVMDY (SEQ ID | | | |
| to | | NO: 740) | | NO: 539) | | | |
| SEQ ID | IMGT | GFSLISYG (SEQ | IWSGGST (SEQ ID NO: | ARNPLTATVMDY (SEQ | | | |
| NO: 63 | | ID NO: 741) | 543) | ID NO: 544) | | | |
| | AbM | GFSLISYGVH | VIWSGGSTD (SEQ ID NO: | NPLTATVMDY (SEQ ID | | | |
| | | (SEQ ID NO: 742) | 546) | NO: 539) | | | |
| | Contact | ISYGVH (SEQ ID | WLGVIWSGGSTD (SEQ ID | ARNPLTATVMD (SEQ | | | |
| | | NO: 743) | NO: 548) | ID NO: 549) | | | |
| Humanized | Kabat | | | | RASQNVGTNVA (SEQ | SASYRDS (SEQ | QQYNSYPLT (SEQ ID |
| 5G8 VL L2 | | | | | ID NO: 780) | ID NO: 744) | NO: 552) |
| corresponding | Chothia | | | | RASQNVGTNVA (SEQ | SASYRDS (SEQ | QQYNSYPLT (SEQ ID |
| to | | | | | ID NO: 780) | ID NO: 744) | NO: 552) |
| SEQ ID | IMGT | | | | QNVGTN (SEQ ID NO: | SAS | QQYNSYPLT (SEQ ID |
| NO: 64 | | | | | 553) | | NO: 552) |
| | AbM | | | | RASQNVGTNVA (SEQ | SASYRDS (SEQ | QQYNSYPLT (SEQ ID |
| | | | | | ID NO: 780) | ID NO: 744) | NO: 552) |
| | Contact | | | | GTNVAWY (SEQ ID NO: | ALIYSASYRD | QQYNSYPL (SEQ ID |
| | | | | | 554) | (SEQ ID NO: | NO: 556) |
| | | | | | | 745) | |
| Humanized | Kabat | | | | RASQNVGTNVA (SEQ | SASYRDS (SEQ | QQYNSYPLT (SEQ ID |
| 5G8 VL L3 | | | | | ID NO: 780) | ID NO: 744) | NO: 552) |
| corresponding | Chothia | | | | RASQNVGTNVA (SEQ | SASYRDS (SEQ | QQYNSYPLT (SEQ ID |
| to | | | | | ID NO: 780) | ID NO: 744) | NO: 552) |
| SEQ ID | IMGT | | | | QNVGTN (SEQ ID NO: | SAS | QQYNSYPLT (SEQ ID |
| NO: 65 | | | | | 553) | | NO: 552) |
| | AbM | | | | RASQNVGTNVA (SEQ | SASYRDS (SEQ | QQYNSYPLT (SEQ ID |
| | | | | | ID NO: 780) | ID NO: 744) | NO: 552) |
| | Contact | | | | GTNVAWY (SEQ ID NO: | ALIYSASYRD | QQYNSYPL (SEQ ID |
| | | | | | 554) | (SEQ ID NO: | NO: 556) |
| | | | | | | 745) | |
| Humanized | Kabat | | | | RASQNVGTNVA (SEQ | SASYRDS (SEQ | QQYNSYPLT (SEQ ID |
| 5G8 VL L4 | | | | | ID NO: 780) | ID NO: 744) | NO: 552) |
| corresponding | Chothia | | | | RASQNVGTNVA (SEQ | SASYRDS (SEQ | QQYNSYPLT (SEQ ID |
| to | | | | | ID NO: 780) | ID NO: 744) | NO: 552) |
| SEQ ID | IMGT | | | | QNVGTN (SEQ ID NO: | SAS | QQYNSYPLT (SEQ ID |
| NO: 66 | | | | | 553) | | NO: 552) |
| | AbM | | | | RASQNVGTNVA (SEQ | SASYRDS (SEQ | QQYNSYPLT (SEQ ID |
| | | | | | ID NO: 780) | ID NO: 744) | NO: 552) |
| | Contact | | | | GTNVAWY (SEQ ID NO: | ALIYSASYRD | QQYNSYPL (SEQ ID |
| | | | | | 554) | (SEQ ID NO: | NO: 556) |
| | | | | | | 745) | |
| Mouse 1C8 | Kabat | TYAIS (SEQ ID | IIWPGGGTNYNSALKS | GAGTWYFDV (SEQ ID | | | |
| VH | | NO: 751) | (SEQ ID NO: 786) | NO: 753) | | | |
| corresponding | Chothia | GFSLTTY (SEQ ID | WPGGG (SEQ ID NO: 656) | GAGTWYFDV (SEQ ID | | | |
| to | | NO: 754) | | NO: 753) | | | |
| SEQ ID NO: 7 | IMGT | GFSLTTYA (SEQ | IWPGGGT (SEQ ID NO: | ARGAGTWYFDV (SEQ | | | |
| | | ID NO: 755) | 756) | ID NO: 757) | | | |
| | AbM | GFSLTTYAIS | IIWPGGGTN (SEQ ID NO: | GAGTWYFDV (SEQ ID | | | |
| | | (SEQ ID NO: 758) | 759) | NO: 753) | | | |
| | Contact | TTYAIS (SEQ ID | WLGIIWPGGGTN (SEQ ID | ARGAGTWYFD (SEQ ID | | | |
| | | NO: 760) | NO: 783) | NO: 762) | | | |
| Mouse 1C8 | Kabat | | | | SASQGISNYLN (SEQ ID | YTSSLHS (SEQ | QQYSKFPWT (SEQ ID |
| VL | | | | | NO: 665) | ID NO: 746) | NO: 770) |
| corresponding | Chothia | | | | SASQGISNYLN (SEQ ID | YTSSLHS (SEQ | QQYSKFPWT (SEQ ID |
| to | | | | | NO: 665) | ID NO: 746) | NO: 770) |
| SEQ ID NO: 8 | IMGT | | | | QGISNY (SEQ ID NO: | YTS | QQYSKFPWT (SEQ ID |
| | | | | | 668) | | NO: 770) |
| | AbM | | | | SASQGISNYLN (SEQ ID | YTSSLHS (SEQ | QQYSKFPWT (SEQ ID |
| | | | | | NO: 665) | ID NO: 746) | NO: 770) |
| | Contact | | | | SNYLNWY (SEQ ID NO: | LLIYYTSSLH | QQYSKFPW (SEQ ID |
| | | | | | 669) | (SEQ ID NO: | NO: 771) |
| | | | | | | 670) | |
| Mouse 1G1 | Kabat | EFGMH (SEQ ID | YISSGGSTIYYADTVKG | DWVDY (SEQ ID NO: | | | |
| VH | | NO: 616) | (SEQ ID NO: 787) | 618) | | | |
| corresponding | Chothia | GFTFSEF (SEQ ID | SSGGST (SEQ ID NO: 764) | DWVDY (SEQ ID NO: | | | |
| to | | NO: 619) | | 618) | | | |
| SEQ ID NO: 9 | IMGT | GFTFSEFG (SEQ | ISSGGSTI (SEQ ID NO: | ARDWVDY (SEQ ID NO: | | | |
| | | ID NO: 621) | 765) | 623) | | | |
| | AbM | GFTFSEFGMH | YISSGGSTIY (SEQ ID NO: | DWVDY (SEQ ID NO: | | | |
| | | (SEQ ID NO: 624) | 766) | 618) | | | |
| | Contact | SEFGMH (SEQ ID | WVAYISSGGSTIY (SEQ ID | ARDWVD (SEQ ID NO: | | | |
| | | NO: 626) | NO: 785) | 628) | | | |

TABLE 19
VH and VL Composition of Humanized 8K22 antibody Variants

| Variant | | |
|---|---|---|
| No | Description | Composition |
| 23820 | 8K22 rabbit - human | HL |
| | parental chimera | |
| 23794 | Humanized 8K22 variant | H1L1 |
| 23795 | Humanized 8K22 variant | H2L1 |
| 23796 | Humanized 8K22 variant | H3L1 |
| 23797 | Humanized 8K22 variant | H4L1 |
| 23798 | Humanized 8K22 variant | H5L1 |
| 23799 | Humanized 8K22 variant | H1L2 |
| 23800 | Humanized 8K22 variant | H2L2 |
| 23801 | Humanized 8K22 variant | H3L2 |
| 23802 | Humanized 8K22 variant | H4L2 |
| 23803 | Humanized 8K22 variant | H5L2 |
| 23804 | Humanized 8K22 variant | H1L3 |
| 23805 | Humanized 8K22 variant | H2L3 |
| 23806 | Humanized 8K22 variant | H3L3 |
| 23807 | Humanized 8K22 variant | H4L3 |
| 23808 | Humanized 8K22 variant | H5L3 |
| 23809 | Humanized 8K22 variant | H1L4 |
| 23810 | Humanized 8K22 variant | H2L4 |
| 23811 | Humanized 8K22 variant | H3L4 |
| 23812 | Humanized 8K22 variant | H4L4 |
| 23813 | Humanized 8K22 variant | H5L4 |
| 23814 | Humanized 8K22 variant | H1L5 |
| 23815 | Humanized 8K22 variant | H2L5 |
| 23816 | Humanized 8K22 variant | H3L5 |
| 23817 | Humanized 8K22 variant | H4L5 |
| 23818 | Humanized 8K22 variant | H5L5 |

TABLE 20
Amino acid sequences of heavy and light chain humanized 8K22

| SEQ | | |
|---|---|---|
| ID | | |
| NO | DESCRIPTION | SEQUENCE |
| 298 | Rabbit 8K22 heavy chain | Provided in Table 17 |
| | variable domain sequence | |
| | (H) | |
| 306 | Heavy chain rabbit 8K22 | EVQLVESGGGLVQPGGSLRLSCAASGFTISNNYYMCWVRQAPGKGLEWVSCIYGGISGRTYYADSVKG |
| | CDRs ported onto framework | RFTISRDNSKNTLYLQMNSLRAEDTAVYYCARGYVGTSNLWGQGTLVTVSS |
| | of germline | |
| | IGHV3-66*01 (H0) | |
| | (also provided in FIG. 40) | |
| 307 | Humanized 8K22 heavy chain | EVQLVESGGGLVQPGGSLRLSCAASGFTISNNYYMCWVRQAPGKGLEWVSCIYGGISGRTYYADSVKG |
| | variable domain sequence 1 | RFTISKDNSKNTLYLQMNSLRAEDTAVYYCVRGYVGTSNLWGQGTLTVSS |
| | (H1) | |
| | (SEQ ID NO: 307) | |
| 308 | Humanized 8K22 heavy chain | EVQLVESGGGLVQPGGSLRLSCAASGFTISNNYYMCWVRQAPGKGLEWIACIYGGISGRTYYADSAKG |
| | variable domain sequence 2 | RFTISKDNSKNTVYLQMNSLRAEDTAVYYCVRGYVGTSNLWGQGTLVTVSS |
| | (H2) | |
| 309 | Humanized 8K22 heavy chain | EVQLVESGGLVQPGGSLRLSCAASGFTISNNYYMCWVRQAPGKGLEWIACIYGGISGRTYYADSAKG |
| | variable domain sequence 3 | RFTISKDSSKNTVYLQMNSLRAEDTAVYYCVRGYVGTSNLWGQGTLVTVSS |
| | (H3) | |
| 310 | Humanized 8K22 heavy chain | EVQLVESGGGLVQPGGSLRLSCAASGFTISNNYYMCWVRQAPGKGLEWIACIYGGISGRTYYADSAKG |
| | variable domain sequence 4 | RFTISKDSSNTVYLQMNSLRAEDTAVYYCVRGYVGTSNLWGQGTLVTVSS |
| | (H4) | |
| 311 | Humanized 8K22 heavy chain | EVQLVESGGGLVQPGGSLRLSCAASGFTISNNYYMCWVRQAPGKGLEWIACIYGGISGRTYYADWAK |
| | variable domain sequence 5 | GRFTISKDSSKNTVYLQMNSLRAEDTAVYYCVRGYVGTSNLWGQGTLVTVSS |
| | (H5) | |
| 299 | Rabbit 8K22 light chain | Provided in Table 17 |
| | variable domain sequence | |
| | (L) | |
| 312 | Light chain rabbit 8K22 | DIQMTQSPSSLSASVGDRVTITCQASQSIYSSLAWYQQKPGKAPKLLIYDASHLASGVPSRFSGSGSGTD |
| | CDRs ported onto | FTLTISSLQPEDFATYYCQGGWYSSAATYVPNTFGGGTKLEIK |
| | framework of germline | |
| | IGKV1-39*01 (L0) | |
| | (also provided in FIG. 40) | |
| 313 | Humanized 8K22 light chain | DIQMTQSPSSLSASVGDRVTITCQASQSIYSSLAWYQQKPGKAPKLLIYDASHLASGVPSRFSGSGSGTD |
| | variable domain sequence 1 | FTLTISSVQPEDFATYYCQGGWYSSAATYVPNTFGGGTKVEIK |
| | (L1)) | |
| 314 | Humanized 8K22 light chain | DIQMTQSPSSLSASVGDRVTITCQASQSIYSSLAWYQQKPGKAPKLLIYDASHLASGVPSRFSGSGSGTD |
| | variable domain sequence | FTLTISSVQPEDFATYYCQGGWYSSAATYVPNTFGGGTKVEVK |
| | 2(L2) | |
| 315 | Humanized 8K22 light chain | DIQMTQSPSSLSASVGDRVTITCQASQSIYSSLAWYQQKPGKAPKLLIYDASHLASGVPSRFSGSRYGTD |
| | variable domain sequence 3 | FTLTISSVQPEDFATYYCQGGWYSSAATYVPNTFGGGTKVEVK |
| | (L3) | |
| 316 | Humanized 8K22 light chain | DIQMTQSPSSLSASVGDRVTITCQASQSIYSSLAWYQQKPGKAPKLLIYDASHLASGVPSRFSGSGSGTD |
| | variable domain sequence 4 | FTLTISSVQPEDAATYYCQGGWYSSAATYVPNTFGGGTKVEVK |
| | (L4) | |
| 317 | Humanized 8K22 light chain | DIQMTQSPSSLSASVGDRVTITCQASQSIYSSLAWYQQKPGKAPKLLIYDASHLASGVPSRFSGSRYGTD |
| | variable domain sequence 5 | FTLTISSVQPEDAATYYCQGGWYSSAATYVPNTFGGGTKVEVK |
| | (L5) | |
| 67 | Ckappa domain of IGKC*01 | RTVAAPSVFIFPPSDEQLKSGTASVVCLLNNFYPREAKVQWKVDNALQSGNSQESVTEQDSKDSTYSLS |
| | | STLTLSKADYEKHKVYACEVTHQGLSSPVTKSFNRGEC |
| 318 | IgG1 CH1-hinge-CH2-CH3 | ASTKGPSVFPLAPSSKSTSGGTAALGCLVKDYFPEPVTVSWNSGALTSGVHTFPAVLQSSGLYSLSSVVT |
| | (Domain boundaries);, D1- | VPSSSLGTQTYICNVNHKPSNTKVDKKVEPKSCDKTHTCPPCPAPELLGGPSVFLFPPKPKDTLMISRTPE |
| | P10, CH2; A11-K120, CH3; | VTCVVVDVSHEDPEVKFNWYVDGVEVHNAKTKPREEQYNSTYRVVSVLTVLHQDWLNGKEYKCKVSN |
| | G121-G226), includes | KALPAPIEKTISKAKGQPREPQVYTLPPSRDELTKNQVSLTCLVKGFYPSDIAVEWESNGQPENNYKTTP |
| | L234A, L235A, D265S | PVLDSDGSFFLYSKLTVDKSRWQQGNVFSCSVMHEALHNHYTQKSLSLSPG |
| | substitutions | |
| 319 | IgG1 Fc sequence 231-446 | APELLGGPSVFLFPPKPKDTLMISRTPEVTCVVVDVSHEDPEVKFNWYVDGVEVHNAKTKPREEQYNST |
| | (EU-numbering), without | YRVVSVLTVLHQDWLNGKEYKCKVSNKALPAPIEKTISKAKGQPREPQVYTLPPSRDELTKNQVSLTCLV |
| | hinge, includes L234A, | KGFYPSDIAVEWESNGQPENNYKTTPPVLDSDGSFFLYSKLTVDKSRWQQGNVFSCSVMHEALHNHY |
| | L235A, D265S substitutions | TQKSLSLSPG |

TABLE 21
Antigen binding assessment of the humanized
8K22 antibody variants by Octet

| | | Supernatant | Purified (n = 2) | |
|---|---|---|---|---|
| Variant | | (n = 1) | Average | STDev |
| No | Description | KD (M) | KD (M) | KD (M) |
| 23820 | 8K22 rabbit-human | 5.94E−09 | 4.11E−09 | 3.08E−10 |
| | parental chimera | | | |
| 23794 | Humanized 8K22 variant | 1.51E−08 | 7.36E−09 | 6.17E−10 |
| 32795 | Humanized 8K22 variant | 3.57E−08 | ND | ND |
| 23796 | Humanized 8K22 variant | 2.22E−08 | 1.12E−08 | 3.88E−09 |
| 23797 | Humanized 8K22 variant | 1.49E−08 | 1.19E−08 | 2.89E−09 |
| 23798 | Humanized 8K22 variant | 1.19E−08 | 6.85E−09 | 2.42E−09 |
| 23799 | Humanized 8K22 variant | 1.38E−08 | 4.8E−09 | 9.85E−10 |
| 23800 | Humanized 8K22 variant | 3.65E−08 | ND | ND |
| 23801 | Humanized 8K22 variant | 2.21E−08 | 8.66E−09 | 7.19E−10 |
| 23802 | Humanized 8K22 variant | 1.79E−08 | 8.53E−09 | 2.62E−09 |
| 23803 | Humanized 8K22 variant | 3.17E−08 | ND | ND |
| 23804 | Humanized 8K22 variant | 9.29E−09 | 4.66E−09 | 1.5E−09 |
| 23805 | Humanized 8K22 variant | 1.62E−08 | 1.02E−08 | 2.64E−09 |
| 23806 | Humanized 8K22 variant | 1.15E−08 | 7.00E−09 | 4.55E−10 |
| 23807 | Humanized 8K22 variant | 1.09E−08 | 7.07E−09 | 7.64E−10 |
| 23808 | Humanized 8K22 variant | 1.93E−08 | 1.31E−08 | 7.78E−10 |
| 23809 | Humanized 8K22 variant | 1.21E−08 | 9.03E−09 | 3.63E−10 |
| 23810 | Humanized 8K22 variant | 3.5E−08 | ND | ND |
| 23811 | Humanized 8K22 variant | 2.25E−08 | 8.83E−09 | 5.69E−09 |
| 23812 | Humanized 8K22 variant | 1.95E−08 | 8.74E−09 | 3.42E−09 |
| 23813 | Humanized 8K22 variant | 3.46E−08 | ND | ND |
| 23814 | Humanized 8K22 variant | 1.01E−08 | 6.31E−09 | 2.64E−10 |
| 23815 | Humanized 8K22 variant | 1.67E−08 | 1.65E−08 | 5.11E−09 |
| 23816 | Humanized 8K22 variant | 1.04E−08 | 1.06E−08 | 2.98E−09 |
| 23817 | Humanized 8K22 variant | 9.5E−09 | 6.41E−09 | 1.23E−09 |
| 23818 | Humanized 8K22 variant | 1.74E−08 | 6.13E−09 | 2.61E−09 |

ND—not determined

TABLE 22
Thermal stability assessment of the humanized
8K22 antibody variants by DSC

| Variant | | |
|---|---|---|
| No. | Description | Fab Tm (° C.) |
| 23820 | 8K22 rabbit-human | 69.16 |
| | parental chimera | |
| 23794 | Humanized 8K22 variant | 74.21 |
| 23795 | Humanized 8K22 variant | ND |
| 23796 | Humanized 8K22 variant | 71.0, 76.61* |
| 23797 | Humanized 8K22 variant | 75.51 |
| 23798 | Humanized 8K22 variant | 72.61, 77.5* |
| 23799 | Humanized 8K22 variant | 72.99 |
| 23800 | Humanized 8K22 variant | ND |
| 23801 | Humanized 8K22 variant | 70.0, 75.56* |
| 23802 | Humanized 8K22 variant | 74.56 |
| 23803 | Humanized 8K22 variant | ND |
| 23804 | Humanized 8K22 variant | 69.88 |
| 23805 | Humanized 8K22 variant | 71.37 |
| 23806 | Humanized 8K22 variant | 71.2 |
| 23807 | Humanized 8K22 variant | 70.46 |
| 23808 | Humanized 8K22 variant | 69.55 |
| 23809 | Humanized 8K22 variant | 77.35 |
| 23810 | Humanized 8K22 variant | ND |
| 23811 | Humanized 8K22 variant | 81.07 |
| 23812 | Humanized 8K22 variant | 79.18 |
| 23813 | Humanized 8K22 variant | ND |
| 23814 | Humanized 8K22 variant | 73.91 |
| 23815 | Humanized 8K22 variant | 75.84 |
| 23816 | Humanized 8K22 variant | 75.48 |
| 23817 | Humanized 8K22 variant | 75.19 |
| 23818 | Humanized 8K22 variant | 71.41, 75.0* |

ND—not determined,
*exhibited two-state transition

TABLE X
Variant Clone Composition

| Variant | H1 clone # | L1 clone # | H2 clone # | L2 clone # |
|---|---|---|---|---|
| 12592 | 2871 | 2872 | 4667 | 2872 |
| 13725 | 8656 | 8653 | 8659 | 8653 |
| 16976 | 10619 | 8653 | 10620 | 8653 |
| 19353 | 13401 | 11150 | 13401 | 11150 |
| 20021 | 14385 | 14402 | 14385 | 14402 |
| 20022 | 14386 | 14471 | 14386 | 14471 |
| 20023 | 14387 | 14409 | 14387 | 14409 |
| 20024 | 14388 | 14404 | 14388 | 14404 |
| 20025 | 14389 | 14405 | 14389 | 14405 |
| 20026 | 14390 | 14406 | 14390 | 14406 |
| 20027 | 14391 | 14407 | 14391 | 14407 |
| 20028 | 14392 | 14408 | 14392 | 14408 |
| 20029 | 14393 | 14409 | 14393 | 14409 |
| 20030 | 14394 | 14410 | 14394 | 14410 |
| 20031 | 14395 | 14408 | 14395 | 14408 |
| 20032 | 14396 | 14411 | 14396 | 14411 |
| 20033 | 14397 | 14412 | 14397 | 14412 |
| 20034 | 14398 | 14413 | 14398 | 14413 |
| 20035 | 14399 | 14414 | 14399 | 14414 |
| 20036 | 14400 | 14415 | 14400 | 14415 |
| 20037 | 14401 | 14416 | 14401 | 14416 |
| 22639 | 8021 | 8022 | 16833 | 8022 |
| 23646 | 18509 | 14471 | 18519 | 14471 |
| 23649 | 18512 | 14415 | 18522 | 14415 |
| 23651 | 18524 | 14471 | 18519 | 14471 |
| 23656 | 18552 | 14471 | 18519 | 14471 |
| 23657 | 18553 | 14405 | 18520 | 14405 |
| 23658 | 18554 | 14411 | 18521 | 14411 |
| 23659 | 18555 | 14415 | 18522 | 14415 |
| 23660 | 18556 | 14416 | 18523 | 14416 |
| 23661 | 18557 | 14471 | 18519 | 14471 |
| 23662 | 18558 | 14405 | 18520 | 14405 |
| 23663 | 18559 | 14411 | 18521 | 14411 |
| 23664 | 18560 | 14415 | 18522 | 14415 |
| 23665 | 18561 | 14416 | 18523 | 14416 |
| 28683 | 20894 | 20897 | 20894 | 20897 |
| 28684 | 20894 | 20898 | 20894 | 20898 |
| 28685 | 20894 | 20899 | 20894 | 20899 |
| 28686 | 20894 | 20900 | 20894 | 20900 |
| 28687 | 20895 | 20897 | 20895 | 20897 |
| 28688 | 20895 | 20898 | 20895 | 20898 |
| 28689 | 20895 | 20899 | 20895 | 20899 |
| 28690 | 20895 | 20900 | 20895 | 20900 |
| 28691 | 20896 | 20897 | 20896 | 20897 |
| 28692 | 20896 | 20898 | 20896 | 20898 |
| 28693 | 20896 | 20899 | 20896 | 20899 |
| 28694 | 20896 | 20900 | 20896 | 20900 |
| 28695 | 20901 | 20905 | 20901 | 20905 |
| 28696 | 20901 | 20906 | 20901 | 20906 |
| 28697 | 20901 | 20907 | 20901 | 20907 |
| 28698 | 20901 | 20908 | 20901 | 20908 |
| 28699 | 20902 | 20905 | 20902 | 20905 |
| 28700 | 20902 | 20906 | 20902 | 20906 |
| 28701 | 20902 | 20907 | 20902 | 20907 |
| 28702 | 20902 | 20908 | 20902 | 20908 |
| 28703 | 20903 | 20905 | 20903 | 20905 |
| 28704 | 20903 | 20906 | 20903 | 20906 |
| 28705 | 20903 | 20907 | 20903 | 20907 |
| 28706 | 20903 | 20908 | 20903 | 20908 |
| 28707 | 20904 | 20905 | 20904 | 20905 |
| 28711 | 20904 | 20906 | 20904 | 20906 |
| 28712 | 20904 | 20907 | 20904 | 20907 |
| 28713 | 20904 | 20908 | 20904 | 20908 |
| 28717 | 21112 | 20891 | 21112 | 20891 |
| 28719 | 21112 | 20892 | 21112 | 20892 |
| 28720 | 21112 | 20893 | 21112 | 20893 |
| 28721 | 21113 | 20891 | 21113 | 20891 |
| 28722 | 21113 | 20892 | 21113 | 20892 |
| 28723 | 21113 | 20893 | 21113 | 20893 |
| 28724 | 21114 | 20891 | 21114 | 20891 |
| 28725 | 21114 | 20892 | 21114 | 20892 |
| 28726 | 21114 | 20893 | 21114 | 20893 |
| 28727 | 21115 | 20891 | 21115 | 20891 |
| 28728 | 21115 | 20892 | 21115 | 20892 |
| 28730 | 21115 | 20893 | 21115 | 20893 |
| 16675 | 10443 | 8022 | 8021 | 8022 |
| 16679 | 10445 | 8022 | 8021 | 8022 |
| 22630 | 16870 | 8022 | 8021 | 8022 |
| 22635 | 16840 | 8022 | 8021 | 8022 |
| 22636 | 16841 | 8022 | 8021 | 8022 |
| 22638 | 16843 | 8022 | 8021 | 8022 |
| 22329 | 16855 | 8022 | 16833 | 8022 |
| 22341 | 16861 | 8022 | 16839 | 8022 |
| 22345 | 16863 | 8022 | 16841 | 8022 |
| 22353 | 16866 | 8022 | 16870 | 8022 |

TABLE XI
Variant Clone Composition

| Variant | H1 clone # | L1 clone # | H2 clone # | L2 clone # | L3 clone # |
|---|---|---|---|---|---|
| 31330 | 22776 | 20898 | 22777 | 20898 | |
| 31331 | 22775 | 20898 | 21709 | 20898 | |
| 31332 | 22776 | 20898 | 21709 | 20898 | |
| 31333 | 22776 | 20898 | 22782 | | |
| 31334 | 22043 | 20898 | 22782 | | |
| 31335 | 22776 | 20898 | 12153 | | |
| 31354 | 22789 | 20898 | 21709 | 20898 | |
| 31362 | 22791* | 20898 | 21709 | 20898 | 21663** |
| 30035 | 21708 | 20891 | 21828 | | |

*N-terminus heavy chain attached to a C-terminus light chain
**This clone is a heavy chain Fab. It should pair with the C-terminus light chain of the H1 clone

TABLE Y
Sequences

| SEQ | | |
|---|---|---|
| ID | Description or | |
| NO: | clone number | Sequence |
| 70 | Human 4-1BB_Fc | GLQDPCSNCPAGTFCDNNRNQICSPCPPNSFSSAGGQRTCDICRQCKGVFRT |
| | | RKECSSTSNAECDCTPGFHCLGAGCSMCEQDCKQGQELTKKGCKDCCFGT |
| | | FNDQKRGICRPWTNCSLDGKSVLVNGTKERDVVCGPSPADLSPGASSVTPP |
| | | APAREPGHSPQDIEGRMDPKSCDKTHTCPPCPAPELLGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGIPREPQVYT |
| | | LPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGKGGHH |
| | | HHHHHHHH |
| 79 | human 4-1BB | MGNSCYNIVATLLLVLNFERTRSLQDPCSNCPAGTFCDNNRNQICSPCPPNS |
| | polypeptide | FSSAGGQRTCDICRQCKGVFRTRKECSSTSNAECDCTPGFHCLGAGCSMCE |
| | (Uniprot | QDCKQGQELTKKGCKDCCFGTFNDQKRGICRPWTNCSLDGKSVLVNGTK |
| | Accession No. | ERDVVCGPSPADLSPGASSVTPPAPAREPGHSPQIISFFLALTSTALLFLLFFL |
| | Q07011) | TLRFSVVKRGRKKLLYIFKQPFMRPVQTTQEEDGCSCRFPEEEEGGCEL |
| 80 | human FRα | MAQRMTTQLLLLLVWVVAVVGEAQTRIAWARTELLNVCMNAKHHKEKPG |
| | polypeptide | PEDKLHEQCRPWRKNACCSTNTSQEAHKDVSYLYRFNWNHCGEMAPACK |
| | (UniProt | RHFIQDTCLYECSPNLGPWIQQVDQSWRKERVLNVPLCKEDCEQWWEDCR |
| | Accession No. | TSYTCKSNWHKGWNWTSGFNKCAVGAACQPFHFYFPTPTVLCNEIWTHS |
| | P15328) | YKVSNYSRGSGRCIQMWFDPAQGNPNEEVARFYAAAMSGAGPWAAWPFL |
| | | LSLALMLLWLLS |
| 81 | human NaPi2b | MAPWPELGDAQPNPDKYLEGAAGQQPTAPDKSKETNKTDNTEAPVTKIEL |
| | polypeptide | LPSYSTATLIDEPTEVDDPWNLPTLQDSGIKWSERDTKGKILCFFQGIGRLIL |
| | (UniProt | LLGFLYFFVCSLDILSSAFQLVGGKMAGQFFSNSSIMSNPLLGLVIGVLVTV |
| | Accession No. | LVQSSSTSTSIVVSMVSSSLLTVRAAIPIIMGANIGTSITNTIVALMQVGDRSE |
| | O95436) | FRRAFAGATVHDFFNWLSVLVLLPVEVATHYLEIITQLIVESFHFKNGEDAP |
| | | DLLKVITKPFTKLIVQLDKKVISQIAMNDEKAKNKSLVKIWCKTFTNKTQIN |
| | | VTVPSTANCTSPSLCWTDGIQNWTMKNVTYKENIAKCQHIFVNFHLPDLA |
| | | VGTILLILSLLVLCGCLIMIVKILGSVLKGQVATVIKKTINTDFPFPFAWLTG |
| | | YLAILVGAGMTFIVQSSSVFTSALTPLIGIGVITIERAYPLTLGSNIGTTTTAIL |
| | | AALASPGNALRSSLQIALCHFFFNISGILLWYPIPFTRLPIRMAKGLGNISAK |
| | | YRWFAVFYLIIFFFLIPLTVFGLSLAGWRVLVGVGVPVVFIIILVLCLRLLQS |
| | | RCPRVLPKKLQNWNFLPLWMRSLKPWDAVVSKFTGCFQMRCCCCCRVCC |
| | | RACCLLCDCPKCCRCSKCCEDLEEAQEGQDVPVKAPETFDNITISREAQGE |
| | | VPASDSKTECTAL |
| 82 | human HER2 | MELAALCRWGLLLALLPPGAASTQVCTGTDMKLRLPASPETHLDMLRHLY |
| | polypeptide | QGCQVVQGNLELTYLPTNASLSFLQDIQEVQGYVLIAHNQVRQVPLQRLRI |
| | (UniProt | VRGTQLFEDNYALAVLDNGDPLNNTTPVTGASPGGLRELQLRSLTEILKGG |
| | Accession No. | VLIQRNPQLCYQDTILWKDIFHKNNQLALTLIDTNRSRACHPCSPMCKGSR |
| | P04626) | CWGESSEDCQSLTRTVCAGGCARCGPLPTDCCHEQCAAGCTGPKHSDCL |
| | | ACLHFNHSGICELHCPALVTYNTDTFESMPNPEGRYTFGASCVTACPYNYL |
| | | STDVGSCTLVCPLHNQEVTAEDGTQRCEKCSKPCARVCYGLGMEHLREVR |
| | | AVTSANIQEFAGCKKIFGSLAFLPESFDGDPASNTAPLQPEQLQVFETLEEIT |
| | | GYLYISAWPDSLPDLSVFQNLQVIRGRILHNGAYSLTLQGLGISWLGLRSLR |
| | | ELGSGLALIHHNTHLCFVHTVPWDQLFRNPHQALLHTANRPEDECVGEGL |
| | | ACHQLCARGHCWGPGPTQCVNCSQFLRGQECVEECRVLQGLPREYVNAR |
| | | HCLPCHPECQPQNGSVTCFGPEADQCVACAHYKDPPFCVARCPSGVKPDLS |
| | | YMPIWKFPDEEGACQPCPINCTHSCVDLDDKGCPAEQRASPLTSIISAVVGI |
| | | LLVVVLGVVFGILIKRRQQKIRKYTMRRLLQETELVEPLTPSGAMPNQAQM |
| | | RILKETELRKVKVLGSGAFGTVYKGIWIPDGENVKIPVAIKVLRENTSPKAN |
| | | KEILDEAYVMAGVGSPYVSRLLGICLTSTVQLVTQLMPYGCLLDHVRENR |
| | | GRLGSQDLLNWCMQIAKGMSYLEDVRLVHRDLAARNVLVKSPNHVKITD |
| | | FGLARLLDIDETEYHADGGKVPIKWMALESILRRRFTHQSDVWSYGVTVW |
| | | ELMTFGAKPYDGIPAREIPDLLEKGERLPQPPICTIDVYMIMVKCWMIDSEC |
| | | RPRFRELVSEFSRMARDPQRFVVIQNEDLGPASPLDSTFYRSLLEDDDMGD |
| | | LVDAEEYLVPQQGFFCPDPAPGAGGMVHHRHRSSSTRSGGGDLTLGLEPSE |
| | | EEAPRSPLAPSEGAGSDVFDGDLGMGAAKGLQSLPTHDPSPLQRYSEDPTV |
| | | PLPSETDGYVAPLTCSPQPEYVNQPDVRPQPPSPREGPLPAARPAGATLERP |
| | | KTLSPGKNGVVKDVAFGGAVENPEYLTPQGGAAPQPHPPPAFSPAFDNLY |
| | | YWDQDPPERGAPPSTFKGTPTAENPEYLGLDVPV |
| 83 | human LIV-1 | MARKLSVILILTFALSVTNPLHELKAAAFPQTTEKISPNWESGINVDLAISTR |
| | polypeptide | QYHLQQLFYRYGENNSLSVEGFRKLLQNIGIDKIKRIHIHHDHDHHSDHEH |
| | (UniProt | HSDHERHSDHEHHSEHEHHSDHDHHSHHNHAASGKNKRKALCPDHDSDS |
| | Accession No. | SGKDPRNSQGKGAHRPEHASGRRNVKDSVSASEVTSTVYNTVSEGTHFLE |
| | Q13433) | TIETPRPGKLFPKDVSSSTPPSVTSKSRVSRLAGRKTNESVSEPRKGFMYSRN |
| | | TNENPQECFNASKLLTSHGMGIQVPLNATEFNYLCPAIINQIDARSCLIHTSE |
| | | KKAEIPPKTYSLQIAWVGGFIAISIISFLSLLGVILVPLMNRVFFKFLLSFLVA |
| | | LAVGTLSGDAFLHLLPHSHASHHHSHSHEEPAMEMKRGPLFSHLSSQNIEE |
| | | SAYFDSTWKGLTALGGLYFMFLVEHVLTLIKQFKDKKKKNQKKPENDDD |
| | | VEIKKQLSKYESQLSTNEEKVDTDDRTEGYLRADSQEPSHFDSQQPAVLEE |
| | | EEVMIAHAHPQEVYNEYVPRGCKNKCHSHFHDTLGQSDDLIHHHHDYHHI |
| | | LHHHHHQNHHPHSHSQRYSREELKDAGVATLAWMVIMGDGLHNFSDGLA |
| | | IGAAFTEGLSSGLSTSVAVFCHELPHELGDFAVLLKAGMTVKQAVLYNALS |
| | | AMLAYLGMATGIFIGHYAENVSMWIFALTAGLFMYVALVDMVPEMLHND |
| | | ASDHGCSRWGYFFLQNAGMLLGFGIMLLISIFEHKIVFRINF |
| 84 | Mesothelin | MALPTARPLLGSCGTPALGSLLFLLFSLGWVQPSRTLAGETGQEAAPLDGV |
| | polypeptide | LANPPNISSLSPRQLLGFPCAEVSGLSTERVRELAVALAQKNVKLSTEQLRC |
| | (UniProt | LAHRLSEPPEDLDALPLDLLLFLNPDAFSGPQACTRFFSRITKANVDLLPRG |
| | Accession No. | APERQRLLPAALACWGVRGSLLSEADVRALGGLACDLPGRFVAESAEVLL |
| | Q13421) | PRLVSCPGPLDQDQQEAARAALQGGGPPYGPPSTWSVSTMDALRGLLPVL |
| | | GQPIIRSIPQGIVAAWRQRSSRDPSWRQPERTILRPRFRREVEKTACPSGKKA |
| | | REIDESLIFYKKWELEACVDAALLATQMDRVNAIPFTYEQLDVLKHKLDEL |
| | | YPQGYPESVIQHLGYLFLKMSPEDIRKWNVTSLETLKALLEVNKGHEMSPQ |
| | | APRRPLPQVATLIDRFVKGRGQLDKDTLDTLTAFYPGYLCSLSPEELSSVPP |
| | | SSIWAVRPQDLDTCDPRQLDVLYPKARLAFQNMNGSEYFVKIQSFLGGAPT |
| | | EDLKALSQQNVSMDLATFMKLRTDAVLPLTVAEVQKLLGPHVEGLKAEER |
| | | HRPVRDWILRQRQDDLDTLGLGLQGGIPNGYLVLDLSMQEALSGTPCLLGP |
| | | GPVLTVLALLLASTLA |
| 86 | 13401 | EVQLVESGGGLVQPGGSLRLSCAASGFNIKDTYIHWVRQAPGKGLEWVAR |
| | | IYPTNGYTRYADSVKGRFTISADTSKNTAYLQMNSLRAEDTAVYYCSRWG |
| | | GDGFYAMDYWGQGTLVTVSSASTKGPSVFPLAPCSRSTSESTAALGCLVK |
| | | DYFPEPVTVSWNSGALTSGVHTFPAVLQSSGLYSLSSVVTVPSSSLGTKTYT |
| | | CNVDHKPSNTKVDKRVESKYGPPCPPCPAPEAAGGPSVFLFPPKPKDTLMIS |
| | | RTPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDSDGSFF |
| | | LYSRLTVDKSRWQEGNVFSCSVMHEALHNHYTQKSLSLSLGKGGGGSGG |
| | | GGSGGGGSQDSTSDLIPAPPLSKVPLQQNFQDNQFHGKWYVVGQAGNIRL |
| | | REDKDPIKMMATIYELKEDKSYDVTMVKFDDKKCMYDIWTFVPGSQPGEF |
| | | TLGKIKSFPGHTSSLVRVVSTNYNQHAMVFFKFVFQNREEFYITLYGRTKEL |
| | | TSELKENFIRFSKSLGLPENHIVFPVPIDQCIDG |
| 87 | 13401 | GAGGTGCAGCTGGTGGAGAGCGGCGGCGGCCTGGTGCAGCCCGGCGGC |
| | | TCTCTGCGGCTGAGCTGCGCCGCCTCCGGCTTTAACATCAAGGACACAT |
| | | ACATCCACTGGGTGCGGCAGGCCCCCGGCAAGGGCCTGGAGTGGGTGG |
| | | CCAGAATCTATCCTACCAATGGCTACACACGGTATGCCGACTCCGTGAA |
| | | GGGCAGATTCACCATCTCTGCCGATACCAGCAAGAACACAGCCTACCTG |
| | | CAGATGAACAGCCTGCGGGCCGAGGATACAGCCGTGTACTATTGTTCTC |
| | | GCTGGGGCGGCGACGGCTTTTACGCCATGGATTATTGGGGCCAGGGCAC |
| | | CCTGGTGACAGTGAGCTCCGCTAGCACAAAAGGACCCTCTGTCTTTCCA |
| | | CTGGCACCCTGCTCACGATCAACCTCTGAATCAACCGCCGCTCTGGGAT |
| | | GTCTGGTCAAGGACTACTTCCCCGAGCCTGTGACCGTGTCTTGGAACAG |
| | | CGGGGCCCTGACCAGCGGAGTGCACACCTTTCCCGCCGTGCTGCAGAGC |
| | | TCCGGCCTGTACTCTCTGTCTAGCGTGGTGACAGTGCCTTCCTCTAGCCT |
| | | GGGCACCAAGACATATACCTGCAACGTGGACCACAAGCCAAGCAATAC |
| | | CAAGGTCGACAAGCGGGTGGAGTCCAAGTACGGACCACCTTGCCCACC |
| | | ATGTCCGGCGCCAGAGGCCGCCGGAGGACCTAGCGTGTTCCTGTTTCCT |
| | | CCAAAGCCAAAGGACACACTGATGATCAGCAGAACACCAGAGGTGACC |
| | | TGCGTGGTGGTGGACGTGTCCCAGGAGGACCCCGAGGTGCAGTTCAACT |
| | | GGTACGTGGATGGCGTGGAGGTGCACAATGCCAAGACCAAGCCCAGAG |
| | | AGGAGCAGTTTAATAGCACATACAGAGTGGTGTCCGTGCTGACCGTGCT |
| | | GCACCAGGACTGGCTGAACGGCAAGGAGTATAAGTGCAAGGTGTCTAA |
| | | TAAGGGCCTGCCTTCCTCTATCGAGAAGACAATCAGCAAGGCCAAGGG |
| | | CCAGCCTCGCGAACCACAGGTGTACACCCTGCCCCCTTCTCAGGAGGAG |
| | | ATGACAAAGAACCAGGTGAGCCTGACCTGTCTGGTGAAGGGCTTCTATC |
| | | CCTCCGACATCGCCGTGGAGTGGGAGTCTAATGGCCAGCCTGAGAACA |
| | | ATTACAAGACCACACCACCCGTGCTGGACTCCGATGGCTCTTTCTTTCTG |
| | | TATTCTAGGCTGACAGTGGATAAGAGCCGCTGGCAGGAGGGCAACGTG |
| | | TTTTCTTGCAGCGTGATGCACGAGGCCCTGCACAATCACTACACCCAGA |
| | | AGTCCCTGAGCTTAAGCCTGGGCAAGGGAGGAGGAGGCAGCCTGCCTGAG |
| | | GAGGCTCCGGCGGCGGCGGCTCTCAGGACTCCACCTCTGATCTGATCCC |
| | | AGCCCCTCCACTGTCCAAGGTGCCCCTGCAGCAGAACTTCCAGGACAAT |
| | | CAGTTTCACGGCAAGTGGTACGTGGTGGGCCAGGCCGGAAACATCCGG |
| | | CTGAGAGAGGACAAGGACCCCATCAAGATGATGGCCACAATCTACGAG |
| | | CTGAAGGAGGACAAGAGCTATGATGTGACCATGGTGAAGTTCGACGAT |
| | | AAGAAGTGTATGTACGATATCTGGACATTTGTGCCAGGCTCCCAGCCTG |
| | | GAGAGTTCACCCTGGGCAAGATCAAGTCTTTTCCTGGCCACACAAGCTC |
| | | CCTGGTGAGGGTGGTGTCCACCAACTATAATCAGCACGCCATGGTGTTC |
| | | TTTAAGTTCGTGTTTCAGAACAGGGAGGAGTTCTACATCACCCTGTATG |
| | | GCCGCACAAAGGAGCTGACCAGCGAGCTGAAGGAGAATTTCATCCGCT |
| | | TTAGCAAGTCCCTGGGGCTGCCAGAGAACCACATTGTCTTTCCAGTGCC |
| | | TATTGACCAGTGTATTGATGGG |
| 88 | 14385 | QVQLKQSGPGLVQPSQSLSITCTVSGFSLTSYGVHWVRQSPGKGLEWLGVI |
| | | WRGGSTDYNAAFISRLSISKDNSKSQVFFKMNSLQADDTAIYYCARENYDY |
| | | DEFAYWGQGTLVTVSAASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 89 | 14385 | CAGGTGCAGCTGAAGCAGAGCGGCCCCGGCCTGGTGCAGCCTAGCCAG |
| | | TCCCTGTCTATCACCTGCACAGTGTCCGGCTTCTCTCTGACCAGCTACGG |
| | | AGTGCACTGGGTGCGGCAGTCCCCAGGCAAGGGCCTGGAGTGGCTGGG |
| | | CGTGATCTGGAGGGGAGGCTCCACAGACTATAACGCCGCCTTTATCTCT |
| | | AGACTGAGCATCTCCAAGGATAACTCTAAGAGCCAGGTGTTCTTTAAGA |
| | | TGAACAGCCTGCAGGCCGACGATACAGCCATCTACTATTGTGCCAGGGA |
| | | GAATTACGACTATGATGAGTTTGCCTACTGGGGCCAGGGCACCCTGGTG |
| | | ACAGTGTCCGCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTGTACACTCTGCCTCCATCTCGGGAC |
| | | GAGCTGACTAAGAACCAGGTCAGTCTGACCTGTCTGGTGAAAGGATTCT |
| | | ATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 90 | 14386 | QVQLKESGPGLVAPSQSLSITCTVSGFSLTTYAISWVRQPPGKGLEWLGIIW |
| | | PGGGTNYNSALKSRLSISKDNSRSQVFLKMNSLQTDDTARYYCARGAGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 91 | 14386 | CAGGTGCAGCTGAAGGAGTCCGGACCAGGACTGGTGGCCCCCTCTCAG |
| | | AGCCTGTCCATCACCTGCACAGTGAGCGGCTTTTCCCTGACCACATACG |
| | | CAATCTCTTGGGTGCGGCAGCCACCTGGCAAGGGACTGGAGTGGCTGG |
| | | GAATCATCTGGCCAGGAGGAGGCACAAACTATAATTCTGCCCTGAAGA |
| | | GCAGGCTGTCTATCAGCAAGGACAACTCCCGCTCTCAGGTGTTCCTGAA |
| | | GATGAACAGCCTGCAGACCGACGATACAGCAAGGTACTATTGTGCCCG |
| | | GGGGGCAGGGACCTGGTACTTTGACGTGTGGGGGGCAGGGACCACAGT |
| | | GACAGTGAGCTCCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAACCACAGGTGTACACTCTGCCTCCATCTCGGGA |
| | | CGAGCTGACTAAGAACCAGGTCAGTCTGACCTGTCTGGTGAAAGGATTC |
| | | TATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 92 | 14387 | EVQLVESGGGLVKPGGSRKLSCAASGFTFSEFGMHWVRQAPDKGLKWVA |
| | | YISSGGSTIYYADTVKGRFTISRDNAKNTLFLQMTSLRSEDTAMYYCARDW |
| | | VDYWGQGTTLTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSK |
| | | LTVDKSRWQQGNVFSCSVMHEALHNHYTQKSLSLSPG |
| 93 | 14387 | GAGGTGCAGCTGTGGAGTCTGGAGGAGGACTGGTGAAGCCAGGAGGC |
| | | AGCAGAAAGCTGTCCTGCGCAGCCTCTGGCTTCACCTTTAGCGAGTTTG |
| | | GCATGCACTGGGTGAGACAGGCCCCCGACAAGGGCCTGAAGTGGGTGG |
| | | CCTACATCAGCTCCGGCGGCAGCACCATCTACTATGCCGACACAGTGAA |
| | | GGGCCGGTTCACCATCTCCAGAGATAACGCCAAGAATACACTGTTTCTG |
| | | CAGATGACCTCCCTGAGGTCTGAGGATACAGCCATGTACTATTGTGCCC |
| | | GCGACTGGGTGGATTATTGGGGCCAGGGCACCACACTGACCGTGTCTAG |
| | | CGCTAGCACAAAGGGCCCCTCCGTGTTTCCTCTGGCCCCATCCTCTAAG |
| | | TCCACCTCTGGAGGAACAGCCGCCCTGGGCTGTCTGGTGAAGGATTACT |
| | | TCCCTGAGCCAGTGACCGTGTCCTGGAACTCTGGGGCCCTGACCAGCGG |
| | | AGTGCACACATTTCCCGCCGTGCTGCAGAGCTCCGGACTGTACTCCCTG |
| | | TCTAGCGTGGTGACCGTGCCTTCCTCTAGCCTGGGCACCCAGACATATA |
| | | TCTGCAACGTGAATCACAAGCCTTCCAATACAAAGGTCGACAAGAAGG |
| | | TGGAGCCAAAGTCTTGTGATAAGACCCACACATGCCCACCTTGTCCGGC |
| | | GCCAGAGGCCGCCGGAGGACCAAGCGTGTTCCTGTTTCCACCCAAGCCC |
| | | AAGGACACCCTGATGATCTCCCGGACCCCAGAGGTGACATGCGTGGTG |
| | | GTGAGCGTGTCCCACGAGGACCCCGAGGTGAAGTTTAACTGGTACGTG |
| | | GATGGCGTGGAGGTGCACAATGCCAAGACAAAGCCCCGGGAGGAGCAG |
| | | TACAATTCTACCTATAGAGTGGTGAGCGTGCTGACAGTGCTGCACCAGG |
| | | ATTGGCTGAACGGCAAGGAGTATAAGTGTAAGGTGAGCAATAAGGCCC |
| | | TGCCAGCCCCCATCGAGAAGACCATCTCCAAGGCCAAGGGCCAGCCTC |
| | | GCGAACCACAGGTGTACACTCTGCCTCCATCTCGGGACGAGCTGACTAA |
| | | GAACCAGGTCAGTCTGACCTGTCTGGTGAAAGGATTCTATCCCAGCGAT |
| | | ATCGCTGTGGAGTGGGAATCCAATGGCCAGCCTGAGAACAATTACAAG |
| | | ACCACACCCCCTGTGCTGGACTCTGATGGCAGTTTCTTTCTGTATAGTAA |
| | | GCTGACCGTCGATAAATCACGATGGCAGCAGGGGAACGTGTTCAGCTG |
| | | TTCAGTGATGCACGAAGCCCTGCACAACCATTACACCCAGAAGAGCCTG |
| | | AGCCTGTCTCCCGGC |
| 94 | 14388 | EVQLQQSGPELVKPGASVKMSCKASGYTFTSYVMHWVKQKPGQGLEWIG |
| | | YINPYNDGTKYNEKFKGKATLTSDKSSSTAYMELSSLTSEDSAVYYCARLG |
| | | SRGTWFAYWGQGTLVTVSAASTKGPSVFPLAPSSKSTSGGTAALGCLVKD |
| | | YFPEPVTVSWNSGALTSGVHTFPAVLQSSGLYSLSVVTVPSSSLGTQTYIC |
| | | NVNHKPSNTKVDKKVEPKSCDKTHTCPPCPAPEAAGGPSVFLFPPKPKDTL |
| | | MISRTPEVTCVVVSVSHEDPEVKFNWYVDGVEVHNAKTKPREEQYNSTYR |
| | | VVSVLTVLHQDWLNGKEYKCKVSNKALPAPIEKTISKAKGQPREPQVYTLP |
| | | PSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 95 | 14388 | GAGGTGCAGCTGCAGCAGAGCGGACCAGAGCTGGTGAAGCCTGGGGCC |
| | | AGCGTGAAGATGTCTTGCAAGGCCAGCGGCTACACCTTCACATCCTATG |
| | | TGATGCACTGGGTGAAGCAGAAGCCAGGCCAGGGCCTGGAGTGGATCG |
| | | GCTACATCAACCCCTATAATGACGGCACCAAGTACAACGAGAAGTTTA |
| | | AGGGCAAGGCCACCCTGACATCTGATAAGAGCTCCTCTACCGCCTATAT |
| | | GGAGCTGAGCTCCCTGACATCTGAGGACAGCGCCGTGTACTATTGTGCC |
| | | CGGCTGGGCTCCAGAGGCACATGGTTTGCCTACTGGGGCCAGGGCACCC |
| | | TGGTGACAGTGTCTGCCGCTAGCACAAAGGGCCCCTCCGTGTTTCCTCT |
| | | GGCCCCATCCTCTAAGTCCACCTCTGGAGGAACAGCCGCCCTGGGCTGT |
| | | CTGGTGAAGGATTACTTCCCTGAGCCAGTGACCGTGTCCTGGAACTCTG |
| | | GGGCCCTGACCAGCGGAGTGCACACATTTCCCGCCGTGCTGCAGAGCTC |
| | | CGGACTGTACTCCCTGTCTAGCGTGGTGACCGTGCCTTCCTCTAGCCTGG |
| | | GCACCCAGACATATATCTGCAACGTGAATCACAAGCCTTCCAATACAAA |
| | | GGTCGACAAGAAGGTGGAGCCAAAGTCTTGTGATAAGACCCACACATG |
| | | CCCACCTTGTCCGGCGCCAGAGGCCGCCGGAGGACCAAGCGTGTTCCTG |
| | | TTTCCACCCAAGCCCAAGGACACCCTGATGATCTCCCGGACCCCAGAGG |
| | | TGACATGCGTGGTGGTGAGCGTGTCCCACGAGGACCCCGAGGTGAAGT |
| | | TTAACTGGTACGTGGATGGCGTGGAGGTGCACAATGCCAAGACAAAGC |
| | | CCCGGGAGGAGCAGTACAATTCTACCTATAGAGTGGTGAGCGTGCTGA |
| | | CAGTGCTGCACCAGGATTGGCTGAACGGCAAGGAGTATAAGTGTAAGG |
| | | TGAGCAATAAGGCCCTGCCAGCCCCCATCGAGAAGACCATCTCCAAGG |
| | | CCAAGGGCCAGCCTCGCGAACCACAGGTGTACACTCTGCCTCCATCTCG |
| | | GGACGAGCTGACTAAGAACCAGGTCAGTCTGACCTGTCTGGTGAAAGG |
| | | ATTCTATCCCAGCGATATCGCTGTGGAGTGGGAATCCAATGGCCAGCCT |
| | | GAGAACAATTACAAGACCACACCCCCTGTGCTGGACTCTGATGGCAGTT |
| | | TCTTTCTGTATAGTAAGCTGACCGTCGATAAATCACGATGGCAGCAGGG |
| | | GAACGTGTTCAGCTGTTCAGTGATGCACGAAGCCCTGCACAACCATTAC |
| | | ACCCAGAAGAGCCTGAGCCTGTCTCCCGGC |
| 96 | 14389 | QVQLKQSGPGLVQPSQSLSITCTVSGFSLTSYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 97 | 14389 | CAGGTGCAGCTGAAGCAGTCCGGACCAGGACTGGTGCAGCCTTCTCAG |
| | | AGCCTGTCCATCACCTGCACAGTGAGCGGCTTCTCCCTGACCTCTTACG |
| | | GCGTGCACTGGGTGAGGCAGTCTCCTGGCAAGGGACTGGAGTGGCTGG |
| | | GCGTGATCTGGAGCGGAGGCTCCACAGACTATAACGCCGCCTTTATCTC |
| | | TCGCCTGTCTATCAGCAAGGATAACTCCAAGTCTCAGGTGTTCTTTAAG |
| | | ATGAATAGCCTGCAGGCCGACGATACAGCCATCTACTATTGTGCCCGGA |
| | | ATCCCCTGACCGCCACAGTGATGGACTACTGGGGCCAGGGCACCAGCG |
| | | TGACAGTGAGCTCCGCTAGCACAAAGGGCCCCTCCGTGTTTCCTCTGGC |
| | | CCCATCCTCTAAGTCCACCTCTGGAGGAACAGCCGCCCTGGGCTGTCTG |
| | | GTGAAGGATTACTTCCCTGAGCCAGTGACCGTGTCCTGGAACTCTGGGG |
| | | CCCTGACCAGCGGAGTGCACACATTTCCCGCCGTGCTGCAGAGCTCCGG |
| | | ACTGTACTCCCTGTCTAGCGTGGTGACCGTGCCTTCCTCTAGCCTGGGCA |
| | | CCCAGACATATATCTGCAACGTGAATCACAAGCCTTCCAATACAAAGGT |
| | | CGACAAGAAGGTGGAGCCAAAGTCTTGTGATAAGACCCACACATGCCC |
| | | ACCTTGTCCGGCGCCAGAGGCCGCCGGAGGACCAAGCGTGTTCCTGTTT |
| | | CCACCCAAGCCCAAGGACACCCTGATGATCTCCCGGACCCCAGAGGTG |
| | | ACATGCGTGGTGGTGAGCGTGTCCCACGAGGACCCCGAGGTGAAGTTT |
| | | AACTGGTACGTGGATGGCGTGGAGGTGCACAATGCCAAGACAAAGCCC |
| | | CGGGAGGAGCAGTACAATTCTACCTATAGAGTGGTGAGCGTGCTGACA |
| | | GTGCTGCACCAGGATTGGCTGAACGGCAAGGAGTATAAGTGTAAGGTG |
| | | AGCAATAAGGCCCTGCCAGCCCCCATCGAGAAGACCATCTCCAAGGCC |
| | | AAGGGCCAGCCTCGCGAACCACAGGTGTACACTCTGCCTCCATCTCGGG |
| | | ACGAGCTGACTAAGAACCAGGTCAGTCTGACCTGTCTGGTGAAAGGATT |
| | | CTATCCCAGCGATATCGCTGTGGAGTGGGAATCCAATGGCCAGCCTGAG |
| | | AACAATTACAAGACCACACCCCCTGTGCTGGACTCTGATGGCAGTTTCT |
| | | TTCTGTATAGTAAGCTGACCGTCGATAAATCACGATGGCAGCAGGGGA |
| | | ACGTGTTCAGCTGTTCAGTGATGCACGAAGCCCTGCACAACCATTACAC |
| | | CCAGAAGAGCCTGAGCCTGTCTCCCGGC |
| 98 | 14390 | QVQLQQPGAELVRPGASVKLSCKASGYPFTSYWMSWVKQRPEQGLEWIG |
| | | RIDPYDSETHYNQKFKDKAILTVDKSSSTAYMQLSSLTSEDSAVYYCARTY |
| | | YGNYDAMDYWGQGTSVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKDT |
| | | LMISRTPEVTCVVVSVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| 99 | 14390 | CAGGTGCAGCTGCAGCAGCCAGGAGCCGAGCTGGTGCGCCCTGGGGCC |
| | | AGCGTGAAGCTGTCCTGCAAGGCCTCTGGCTACCCCTTCACCAGCTATT |
| | | GGATGTCCTGGGTGAAGCAGCGGCCAGAGCAGGGACTGGAGTGGATCG |
| | | GCAGAATCGACCCCTACGATTCTGAGACACACTATAACCAGAAGTTTAA |
| | | GGACAAGGCCATCCTGACCGTGGATAAGAGCTCCTCTACAGCCTACATG |
| | | CAGCTGAGCTCCCTGACCTCCGAGGACTCTGCCCGTACTATTGTGCCA |
| | | GGACATACTATGGCAATTACGACGCCATGGATTATTGGGGCCAGGGCA |
| | | CCTCCGTGACAGTGTCTAGCGCTAGCACAAAGGGCCCCTCCGTTTCC |
| | | TCTGGCCCCATCCTCTAAGTCCACCTCTGGAGGAACAGCCGCCCTGGGC |
| | | TGTCTGGTGAAGGATTACTTCCCTGAGCCAGTGACCGTGTCCTGGAACT |
| | | CTGGGGCCCTGACCAGCGGAGTGCACACATTTCCCGCCGTGCTGCAGAG |
| | | CTCCGGACTGTACTCCCTGTCTAGCGTGGTGACCGTGCCTTCCTCTAGCC |
| | | TGGGCACCCAGACATATATCTGCAACGTGAATCACAAGCCTTCCAATAC |
| | | AAAGGTCGACAAGAAGGTGGAGCCAAAGTCTTGTGATAAGACCCACAC |
| | | ATGCCCACCTTGTCCGGCGCCAGAGGCCGCCGGAGGACCAAGCGTGTTC |
| | | CTGTTTCCACCCAAGCCCAAGGACACCCTGATGATCTCCCGGACCCCAG |
| | | AGGTGACATGCGTGGTGGTGAGCGTGTCCCACGAGGACCCCGAGGTGA |
| | | AGTTTAACTGGTACGTGGATGGCGTGGAGGTGCACAATGCCAAGACAA |
| | | AGCCCCGGGAGGAGCAGTACAATTCTACCTATAGAGTGGTGAGCGTGC |
| | | TGACAGTGCTGCACCAGGATTGGCTGAACGGCAAGGAGTATAAGTGTA |
| | | AGGTGAGCAATAAGGCCCTGCCAGCCCCCATCGAGAAGACCATCTCCA |
| | | AGGCCAAGGGCCAGCCTCGCGAACCACAGGTGTACACTCTGCCTCCATC |
| | | TCGGGACGAGCTGACTAAGAACCAGGTCAGTCTGACCTGTCTGGTGAA |
| | | AGGATTCTATCCCAGCGATATCGCTGTGGAGTGGGAATCCAATGGCCAG |
| | | CCTGAGAACAATTACAAGACCACACCCCCTGTGCTGGACTCTGATGGCA |
| | | GTTTCTTTCTGTATAGTAAGCTGACCGTCGATAAATCACGATGGCAGCA |
| | | GGGGAACGTGTTCAGCTGTTCAGTGATGCACGAAGCCCTGCACAACCAT |
| | | TACACCCAGAAGAGCCTGAGCCTGTCTCCCGGC |
| 100 | 14391 | QVQLQQPGAELVRPGASVKLSCKASGYTFTSYWINWVKQRPGQGLEWIGN |
| | | IYPSDNYTNYNQKFKDKATLTVDKSSSTAYMQLSSPTSEDSAVYYCTRGG |
| | | GIYYENYFDYWGQGTTLTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNKPSNTKVDKKVEPKSCDKTHTCPPCPAPEAAGGPSVFLFPPKPKDT |
| | | LMISRTPEVTCVVVSVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSRDELTKNQVSLTCLVKGFYRSDIAVEWESNGQPENNYKTTPPVLDSD |
| | | GSFFLYSKLTVDKSRWQQGNVFSCSVMHEALHNHYTQKSLSLSPG |
| 101 | 14391 | CAGGTGCAGCTGCAGCAGCCAGGAGCCGAGCTGGTGAGACCTGGGGCC |
| | | AGCGTGAAGCTGTCCTGCAAGGCCTCTGGCTACACCTTCACAAGCTATT |
| | | GGATCAACTGGGTGAAGCAGAGGCCAGGACAGGGACTGGAGTGGATCG |
| | | GCAACATCTACCCCTCCGACAACTACACAAACTACAACCAGAAGTTTAA |
| | | GGACAAGGCCACCCTGACAGTGGATAAGAGCTCCTCTACCGCCTATATG |
| | | CAGCTGAGCTCCCCCACATCCGAGGACTCTGCCGTGTACTATTGTACCC |
| | | GGGGCGGCGGCATCTACTACGAGAACTACTTCGATTACTGGGGCCAGG |
| | | GCACCACACTGACAGTGTCTAGCGCTAGCACAAAGGGCCCCTCCGTGTT |
| | | TCCTCTGGCCCCATCCTCTAAGTCCACCTCTGGAGGAACAGCCGCCCTG |
| | | GGCTGTCTGGTGAAGGATTACTTCCCTGAGCCAGTGACCGTGTCCTGGA |
| | | ACTCTGGGGCCCTGACCAGCGGAGTGCACACATTTCCCGCCGTGCTGCA |
| | | GAGCTCCGGACTGTACTCCCTGTCTAGCGTGGTGACCGTGCCTTCCTCTA |
| | | GCCTGGGCACCCAGACATATATCTGCAACGTGAATCACAAGCCTTCCAA |
| | | TACAAAGGTCGACAAGAAGGTGGAGCCAAAGTCTTGTGATAAGACCCA |
| | | CACATGCCCACCTTGTCCGGCGCCAGAGGCCGCCGGAGGACCAAGCGT |
| | | GTTCCTGTTTCCACCCAAGCCCAAGGACACCCTGATGATCTCCCGGACC |
| | | CCAGAGGTGACATGCGTGGTGGTGAGCGTGTCCCACGAGGACCCCGAG |
| | | GTGAAGTTTAACTGGTACGTGGATGGCGTGGAGGTGCACAATGCCAAG |
| | | ACAAAGCCCCGGGAGGAGCAGTACAATTCTACCTATAGAGTGGTGAGC |
| | | GTGCTGACAGTGCTGCACCAGGATTGGCTGAACGGCAAGGAGTATAAG |
| | | TGTAAGGTGAGCAATAAGGCCCTGCCAGCCCCCATCGAGAAGACCATC |
| | | TCCAAGGCCAAGGGCCAGCCTCGCGAACCACAGGTGTACACTCTGCCTC |
| | | CATCTCGGGACGAGCTGACTAAGAACCAGGTCAGTCTGACCTGTCTGGT |
| | | GAAAGGATTCTATCCCAGCGATATCGCTGTGGAGTGGGAATCCAATGGC |
| | | CAGCCTGAGAACAATTACAAGACCACACCCCCTGTGCTGGACTCTGATG |
| | | GCAGTTTCTTTCTGTATAGTAAGCTGACCGTCGATAAATCACGATGGCA |
| | | GCAGGGGAACGTGTTCAGCTGTTCAGTGATGCACGAAGCCCTGCACAA |
| | | CCATTACACCCAGAAGAGCCTGAGCCTGTCTCCCGGC |
| 102 | 14392 | QVHLQQSGSELRIPGSSVKLSCKDFDSEVFPIAYMSWVRQKPGHGFEWIGDI |
| | | LPSIGRTIYGEKFEDKATLDADTVSNTAYLDLNSLTSEDSAIYYCSRGDYYY |
| | | GSREYAMDYWGQGTSVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKDT |
| | | LMISRTPEVTCVVVSVSHEDPEVKFNWYNDGVEVHNAKTKPREEQYNSTY |
| | | RVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| 103 | 14392 | CAGGTGCACCTGCAGCAGTCCGGCTCTGAGCTGCGCATCCCTGGCAGCT |
| | | CCGTGAAGCTGAGCTGCAAGGACTTCGATTCCGAGGTGTTTCCCATCGC |
| | | CTATATGTCCTGGGTGCGGCAGAAGCCTGGACACGGATTCGAGTGGATC |
| | | GGCGACATCCTGCCCTCTATCGGCAGGACCATCTATGGCGAGAAGTTTG |
| | | AGGATAAGGCCACACTGGACGCCGATACCGTGTCCAACACAGCCTACC |
| | | TGGACCTGAATAGCCTGACATCCGAGGATTCTGCCATCTACTATTGTTCT |
| | | CGGGGCGACTACTATTACGGCAGCAGAGAGTATGCCATGGATTACTGG |
| | | GGCCAGGGCACCAGCGTGACAGTGTCTAGCGCTAGCACAAAGGGCCCC |
| | | TCCGTGTTTCCTCTGGCCCCATCCTCTAAGTCCACCTCTGGAGGAACAGC |
| | | CGCCCTGGGCTGTCTGGTGAAGGATTACTTCCCTGAGCCAGTGACCGTG |
| | | TCCTGGAACTCTGGGGCCCTGACCAGCGGAGTGCACACATTTCCCGCCG |
| | | TGCTGCAGAGCTCCGGACTGTACTCCCTGTCTAGCGTGGTGACCGTGCC |
| | | TTCCTCTAGCCTGGGCACCCAGACATATATCTGCAACGTGAATCACAAG |
| | | CCTTCCAATACAAAGGTCGACAAGAAGGTGGAGCCAAAGTCTTGTGAT |
| | | AAGACCCACACATGCCCACCTTGTCCGGCGCCAGAGGCCGCCGGAGGA |
| | | CCAAGCGTGTTCCTGTTTCCACCCAAGCCCAAGGACACCCTGATGATCT |
| | | CCCGGACCCCAGAGGTGACATGCGTGGTGGTGAGCGTGTCCCACGAGG |
| | | ACCCCGAGGTGAAGTTTAACTGGTACGTGCATGGCGTGGAGGTGCACA |
| | | ATGCCAAGACAAAGCCCCGGGAGGAGCAGTACAATTCTACCTATAGAG |
| | | TGGTGAGCGTGCTGACAGTGCTGCACCAGGATTGGCTGAACGGCAAGG |
| | | AGTATAAGTGTAAGGTGAGCAATAAGGCCCTGCCAGCCCCCATCGAGA |
| | | AGACCATCTCCAAGGCCAAGGGCCAGCCTCGCGAACCACAGGTGTACA |
| | | CTCTGCCTCCATCTCGGGACGAGCTGACTAAGAACCAGGTCAGTCTGAC |
| | | CTGTCTGGTGAAAGGATTCTATCCCAGCGATATCGCTGTGGAGTGGGAA |
| | | TCCAATGGCCAGCCTGAGAACAATTACAAGACCACACCCCCTGTGCTGG |
| | | ACTCTGATGGCAGTTTCTTTCTGTATAGTAAGCTGACCGTCGATAAATC |
| | | ACGATGGCAGCAGGGGAACGTGTTCAGCTGTTCAGTGATGCACGAAGC |
| | | CCTGCACAACCATTACACCCAGAAGAGCCTGAGCCTGTCTCCCGGC |
| 104 | 14393 | QVQLQESGGGLVKPGGSRKLSCAASGFTFSEFGMHWVRQAPDKGLEWVA |
| | | YISSGSSTIYYADTVKGRFTISRDNAKNTLFLQMTSLRSEDTAMYYCARDW |
| | | VDYWGQGTALTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVEVHNAKTKPREEQYNSTYRVVSVLT |
| | | VLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSK |
| | | LTVDKSRWQQGNVFSCSVMHEALHNHYTQKSLSLSPG |
| 105 | 14393 | CAGGTGCAGCTGCAGGAGTCTGGAGGAGGACTGGTGAAGCCAGGAGGC |
| | | TCTAGAAAGCTGAGCTGCGCCGCCTCCGGCTTCACCTTTAGCGAGTTTG |
| | | GCATGCACTGGGTGAGACAGGCCCCCGACAAGGGACTGGAGTGGGTGG |
| | | CCTACATCAGCTCCGGCTCTAGCACCATCTACTATGCCGACACAGTGAA |
| | | GGGCCGGTTCACCATCTCCAGAGATAACGCCAAGAATACACTGTTTCTG |
| | | CAGATGACCAGCCTGAGGTCCGAGGATACAGCCATGTACTATTGTGCCC |
| | | GCGACTGGGTGGATTATTGGGGACAGGGAACCGCCCTGACAGTGTCCTC |
| | | TGCTAGCACAAAGGGCCCCTCCGTGTTTCCTCTGGCCCCATCCTCTAAGT |
| | | CCACCTCTGGAGGAACAGCCGCCCTGGGCTGTCTGGTGAAGGATTACTT |
| | | CCCTGAGCCAGTGACCGTGTCCTGGAACTCTGGGGCCCTGACCAGCGGA |
| | | GTGCACACATTTCCCGCCGTGCTGCAGAGCTCCGGACTGTACTCCCTGT |
| | | CTAGCGTGGTGACCGTGCCTTCCTCTAGCCTGGGCACCCAGACATATAT |
| | | CTGCAACGTGAATCACAAGCCTTCCAATACAAAGGTCGACAAGAAGGT |
| | | GGAGCCAAAGTCTTGTGATAAGACCCACACATGCCCACCTTGTCCGGCG |
| | | CCAGAGGCCGCCGGAGGACCAAGCGTGTTCCTGTTTCCACCCAAGCCCA |
| | | AGGACACCCTGATGATCTCCCGGACCCCAGAGGTGACATGCGTGGTGGT |
| | | GAGCGTGTCCCACGAGGACCCCGAGGTGAAGTTTAACTGGTACGTGGA |
| | | TGGCGTGGAGGTGCACAATGCCAAGACAAAGCCCCGGGAGGAGCAGTA |
| | | CAATTCTACCTATAGAGTGGTGAGCGTGCTGACAGTGCTGCACCAGGAT |
| | | TGGCTGAACGGCAAGGAGTATAAGTGTAAGGTGAGCAATAAGGCCCTG |
| | | CCAGCCCCCATCGAGAAGACCATCTCCAAGGCCAAGGGCCAGCCTCGC |
| | | GAACCACAGGTGTACACTCTGCCTCCATCTCGGGACGAGCTGACTAAGA |
| | | ACCAGGTCAGTCTGACCTGTCTGGTGAAAGGATTCTATCCCAGCGATAT |
| | | CGCTGTGGAGTGGGAATCCAATGGCCAGCCTGAGAACANTTACAAGAC |
| | | CACACCCCCTGTGCTGGACTCTGATGGCAGTTTCTTTCTGTATAGTAAGC |
| | | TGACCGTCGATAAATCACGATGGCAGCAGGGGAACGTGTTCAGCTGTTC |
| | | AGTGATGCACGAAGCCCTGCACAACCATTACACCCAGAAGAGCCTGAG |
| | | CCTGTCTCCCGGC |
| 106 | 14394 | QVQLKQSGPGLVQPSQSLSITCTVSGFSLTSYGVHWVRQSPGKGLEWLGVI |
| | | WSGGSTDYNGAFISRLSISKDNSKSQVFFKMNSLQADDTAMYFCARDRGG |
| | | GFDYWGQGTTLTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 107 | 14394 | CAGGTGCAGCTGAAGCAGTCCGGCCCCGGCCTGGTGCAGCCTTCTCAGA |
| | | GCCTGTCCATCACCTGCACAGTGAGCGGCTTCTCCCTGACCTCTTACGG |
| | | AGTGCACTGGGTGCGGCAGTCTCCAGGCAAGGGACTGGAGTGGCTGGG |
| | | CGTGATCTGGAGCGGAGGCTCCACAGACTATAACGGGGCCTTCATCTCT |
| | | AGGCTGTCTATCAGCAAGGATAACTCCAAGTCTCAGGTGTTCTTTAAGA |
| | | TGAATAGCCTGCAGGCCGACGATACCGCCATGTACTTCTGTGCCCGGGA |
| | | CAGAGGCGGCGGCTTTGATTATTGGGGCCAGGGCACCACACTGACAGT |
| | | GAGCTCCGCTAGCACAAAGGGCCCCTCCGTGTTTCCTCTGGCCCCATCC |
| | | TCTAAGTCCACCTCTGGAGGAACAGCCGCCCTGGGCTGTCTGGTGAAGG |
| | | ATTACTTCCCTGAGCCAGTGACCGTGTCCTGGAACTCTGGGGCCCTGAC |
| | | CAGCGGAGGCACACATTTCCCGCCGTGCTGCAGAGCTCCGGACTGTAC |
| | | TCCCTGTCTAGCGTGGTGACCGTGCCTTCCTCTAGCCTGGGCACCCAGA |
| | | CATATATCTGCAACGTGAATCACAAGCCTTCCAATACAAAGGTCGACAA |
| | | GAAGGTGGAGCCAAAGTCTTGTGATAAGACCCACACATGCCCACCTTGT |
| | | CCGGCGCCAGAGGCCGCCGGAGGACCAAGCGTGTTCCTGTTTCCACCCA |
| | | AGCCCAAGGACACCCTGATGATCTCCCGGACCCCAGAGGTGACATGCG |
| | | TGGTGGTGAGCGTGTCCCACGAGGACCCCGAGGTGAAGTTTAACTGGTA |
| | | CGTGGATGGCGTGGAGGTGCACAATGCCAAGACAAAGCCCCGGGAGGA |
| | | GCAGTACAATTCTACCTATAGAGTGGTGAGCGTGCTGACAGTGCTGCAC |
| | | CAGGATTGGCTGAACGGCAAGGAGTATAAGTGTAAGGTGAGCAATAAG |
| | | GCCCTGCCAGCCCCCATCGAGAAGACCATCTCCAAGGCCAAGGGCCAG |
| | | CCTCGCGAACCACAGGTGTACACTCTGCCTCCATCTCGGGACGAGCTGA |
| | | CTAAGAACCAGGTCAGTCTGACCTGTCTGGTGAAAGGATTCTATCCCAG |
| | | CGATATCGCTGTGGAGTGGGAATCCAATGGCCAGCCTGAGAACAATTA |
| | | CAAGACCACACCCCCTGTGCTGGACTCTGATGGCAGTTTCTTTCTGTATA |
| | | GTAAGCTGACCGTCGATAAATCACGATGGCAGCAGGGGAACGTGTTCA |
| | | GCTGTTCAGTGATGCACGAAGCCCTGCACAACCATTACACCCAGAAGA |
| | | GCCTGAGCCTGTCTCCCGGC |
| 108 | 4667 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPCSRSTSESTAALGCLVKDYFPEPVT |
| | | VSWNSGALTSGVHTFPAVLQSSGLYSLSSVVTVPSSNFGTQTYTCNVDHKP |
| | | SNTKVDKTVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPG |
| 109 | 4667 | GAAGTCCAGCTGGTCCAGTCCGGAGCCGAGGTGAAGAAACCCGGCGAA |
| | | TCACTGCGAATCAGCTGCAAGGGCAGCGGCTACAGCTTCTCCACTTATT |
| | | GGATTAGCTGGGTGCGGCAGATGCCTGGGAAGGGACTGGAGTGGATGG |
| | | GGAAAATCTACCCCGGAGATAGCTACACCAACTATTCTCCTAGTTTTCA |
| | | GGGCCAAGTGACTATCTCCGCCGACAAGTCAATTAGCACCGCTTATCTG |
| | | CAGTGGAGCTCCCTGAAAGCTAGCGATACAGCAATGTACTATTGTGCCA |
| | | GAGGCTACGGGATTTTCGACTATTGGGGACAGGGCACTCTGGTCACCGT |
| | | GTCTAGTGCTTCTACTAAGGGGCCCAGTGTGTTTCCACTGGCACCCTGCT |
| | | CCAGGTCTACAAGTGAATCAACTGCCGCTCTGGGATGTCTGGTGAAAGA |
| | | TTACTTCCCAGAGCCCGTCACAGTGAGCTGGAACTCCGGCGCACTGACT |
| | | TCTGGGGTCCACACCTTTCCTGCCGTGCTGCAGTCAAGCGGCCTGTACA |
| | | GCCTGTCCTCTGTGGTCACCGTGCCAAGTTCAAATTTCGGGACCCAGAC |
| | | ATATACTTGCAACGTGGACCACAAGCCTTCTAATACAAAGGTCGATAAA |
| | | ACTGTGGAACCAAAGAGTTGTGACAAAACCCATACATGCCCCCCTTGTC |
| | | CTGCACCAGAGCTGCTGGGAGGACCATCCGTGTTCCTGTTTCCACCCAA |
| | | GCCCAAAGATACACTGATGATCAGCCGCACCCCAGAAGTCACATGCGT |
| | | GGTCGTGGACGTGTCCCACGAGGACCCCGAAGTCAAGTTTAACTGGTAC |
| | | GTGGACGGCGTCGAGGTGCATAATGCTAAGACCAAACCACGGGAGGAA |
| | | CAGTACAATTCAACCTATAGAGTCGTGAGCGTCCTGACAGTGCTGCACC |
| | | AGGATTGGCTGAACGGCAAAGAGTATAAGTGCAAAGTGTCTAATAAGG |
| | | CACTGCCCGCCCCTATCGAGAAAACCATTAGCAAGGCCAAAGGGCAGC |
| | | CTAGGGAACCACAGGTCTACGTGCTGCCTCCAAGCCGCGATGAGCTGAC |
| | | AAAGAACCAGGTCTCCCTGCTGTGTCTGGTGAAAGGGTTCTATCCCAGT |
| | | GACATTGCTGTGGAGTGGGAATCAAATGGACAGCCTGAAAACAATTAC |
| | | CTGACATGGCCCCCTGTGCTGGACTCTGATGGAAGTTTCTTTCTGTATTC |
| | | CAAGCTGACTGTGGACAAATCTCGATGGCAGCAGGGCAACGTCTTTAGC |
| | | TGTTCCGTGATGCATGAGGCCCTGCACAATCATTACACCCAGAAGTCTC |
| | | TGAGTCTGTCACCTGGC |
| 110 | 14396 | QVQLKESGPGLVAPSQSLSITCTVSGFSLTSYAINWVRQPPGKGLEWLGVL |
| | | WPGGGTNYNSALKSRLSISKDNSKSQVFLKMNSLQTDDTARYYCARGSGT |
| | | WYFDVWGAGTTVTSSASTKGPSVFLPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 111 | 14396 | CAGGTGCAGCTGAAGGAGAGCGGACCAGGACTGGTGGCCCCCTCTCAG |
| | | AGCCTGTCCATCACCTGCACAGTGAGCGGCTTTTCCCTGACCTCTTACGC |
| | | CATCAACTGGGTGCGGCAGCCACCTGGCAAGGGACTGGAGTGGCTGGG |
| | | CGTGCTGTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAACAGCCTGCAGACCGACGATACAGCCCGGTACTATTGTGCCAGA |
| | | GGCTCCGGCACCTGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCCTAGAAGACCATCTCCAAGGCCAA |
| | | GGGCCAGCCTCGCGAACCACAGGTGTACACTCTGCCTCCATCTCGGGAC |
| | | GAGCTGACTAAGAACCAGGTCAGTCTGACCTGTCTGGTGAAAGGATTCT |
| | | ATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 112 | 14397 | QVQLQQPGAELVKPGASVMLSCKASGYTFTSYWMHWVKQRPGQGLEWI |
| | | GEINLRNGGTNYYEKFKTRATLTVDKSSSTAYMQLSSLTSEDSAVYYCTIL |
| | | TSAPSYWGQGTLVTVSAASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPEAAGGPSVFLFPPKPKDTLMISR |
| | | TPEVTCVVVSVSHEDPEVKFNWYVDGVEVHNAKTKPREEQYNSTYRVVS |
| | | VLTVLHQDWLNGKEYKCKVSNKALPAPIEKTISKAKGQPREPQVYTLPPSR |
| | | DELTKNQVSLTCLVKGFYPSDIAVEWESNGQRENNYKTTPPVLDSDGSFFL |
| | | YSKLTVDKSRWQQGNVFSCSVMHEALHNHYTQKSLSLSPG |
| 113 | 14397 | CAGGTGCAGCTGCAGCAGCCAGGAGCCGAGCTGGTGAAGCCTGGGGCC |
| | | AGCGTGATGCTGTCCTGCAAGGCCTCTGGCTACACCTTCACAAGCTATT |
| | | GGATGCACTGGGTGAAGCAGCGGCCTGGACAGGGACTGGAGTGGATCG |
| | | GCGAGATCAACCTGCGGAATGGCGGCACCAACTACTATGAGAAGTTTA |
| | | AGACAAGAGCCACCCTGACAGTGGACAAGAGCTCCTCTACCGCCTACA |
| | | TGCAGCTGAGCTCCCTGACATCTGAGGATAGCGCCGTGTACTATTGTAC |
| | | CATCCTGACATCCGCCCCCTCTTATTGGGGACAGGGCACCCTGGTGACA |
| | | GTGTCCGCCGCTAGCACAAAGGGCCCCTCCGTGTTTCCTCTGGCCCCAT |
| | | CCTCTAAGTCCACCTCTGGAGGAACAGCCGCCCTGGGCTGTCTGGTGAA |
| | | GGATTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCCAATACAAAGGTCGAC |
| | | AAGAAGGTGGAGCCAAAGTCTTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCCGGACCCCAGAGGTGACAT |
| | | GCGTGGTGGTGAGCGTGTCCCACGAGGACCCCGAGGTGAAGTTTAACT |
| | | GGTACGTGGATGGCGTGGAGGTGCACAATGCCAAGACAAAGCCCCGGG |
| | | AGGAGCAGTACAATTCTACCTATAGAGTGGTGAGCGTGCTGACAGTGCT |
| | | GCACCAGGATTGGCTGAACGGCAAGGAGTATAAGTGTAAGGTGAGCAA |
| | | TAAGGCCCTGCCAGCCCCCATCGAGAAGACCATCTCCAAGGCCAAGGG |
| | | CCAGCCTCGCGAACCACAGGTGTACACTCTGCCTCCATCTCGGGACGAG |
| | | CTGACTAAGAACCAGGTCAGTCTGACCTGTCTGGTGAAAGGATTCTATC |
| | | CCAGCGATATCGCTGTGGAGTGGGAATCCAATGGCCAGCCTGAGAACA |
| | | ATTACAAGACCACACCCCCTGTGCTGGACTCTGATGGCAGTTTCTTTCTG |
| | | TATAGTAAGCTGACCGTCGATAAATCACGATGGCAGCAGGGGAACGTG |
| | | TTCAGCTGTTCAGTGATGCACGAAGCCCTGCACAACCATTACACCCAGA |
| | | AGAGCCTGAGCCTGTCTCCCGGC |
| 114 | 14398 | QLQESGAELLRPGASVKLSCTASGFNIKDDYLHWVKQRPEQGLEWIGWIDP |
| | | ENGDTEYASKFQGKATIKADTSSNTAYLQLSSLTSEDTAVYYCSTQGFACW |
| | | GQGTLVTVSAASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTK |
| | | VDKKVEPKSCDKTHTCPPCPAPEAAGGPSVFLFPPKPKDTLMISRTPEVTCV |
| | | VVSVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLTVD |
| | | KSRWQQGNVFSCSVMHEALHNHYTQKSLSLSPG |
| 115 | 14398 | CAGCTGCAGGAGTCTGGAGCCGAGCTGCTGAGACCCGGGGCCAGCGTG |
| | | AAGCTTGTCTTGCACAGCCAGCGGCTTCAACATCAAGGACGATTACCTGC |
| | | ACTGGGTGAAGCAGAGACCCGAGCAGGGACTGGAGTGGATCGGATGGA |
| | | TCGACCCTGAGAACGGCGATACCGAGTACGCCAGCAAGTTTCAGGGCA |
| | | AGGCCACAATCAAGGCCGACACCAGCTCCAATACAGCCTATCTGCAGCT |
| | | GTCTAGCCTGACCTCCGAGGATACAGCCGTGTACTATTGCTCTACCCAG |
| | | GGATTCGCATGTTGGGGACAGGGCACCCTGGTGACAGTGTCCGCCGCTA |
| | | GCACAAAGGGCCCCTCCGTGTTTCCTCTGGCCCCATCCTCTAAGTCCAC |
| | | CTCTGGAGGAACAGCCGCCCTGGGCTGTCTGGTGAAGGATTACTTCCCT |
| | | GAGCCAGTGACCGTGTCCTGGAACTCTGGGGCCCTGACCAGCGGAGTG |
| | | CACACATTTCCCGCCGTGCTGCAGAGCTCCGGACTGTACTCCCTGTCTA |
| | | GCGTGGTGACCGTGCCTTCCTCTAGCCTGGGCACCCAGACATATATCTG |
| | | CAACGTGAATCACAAGCCTTCCAATACAAAGGTCGACAAGAAGGTGGA |
| | | GCCAAAGTCTTGTGATAAGACCCACACATGCCCACCTTGTCCGGCGCCA |
| | | GAGGCCGCCGGAGGACCAAGCGTGTTCCTGTTTCCACCCAAGCCCAAG |
| | | GACACCCTGATGATCTCCCGGACCCCAGAGGTGACATGCGTGGTGGTGA |
| | | GCGTGTCCCACGAGGACCCCGAGGTGAAGTTTAACTGGTACGTGGATG |
| | | GCGTGGAGGTGCACAATGCCAAGACAAAGCCCCGGGAGGAGCAGTACA |
| | | ATTCTACCTATAGAGTGGTGAGCGTGCTGACAGTGCTGCACCAGGATTG |
| | | GCTGAACGGCAAGGAGTATAAGTGTAAGGTGAGCAATAAGGCCCTGCC |
| | | AGCCCCCATCGAGAAGACCATCTCCAAGGCCAAGGGCCAGCCTCGCGA |
| | | ACCACAGGTGTACACTCTGCCTCCATCTCGGGACGAGCTGACTAAGAAC |
| | | CAGGTCAGTCTGACCTGTCTGGTGAAAGGATTCTATCCCAGCGATATCG |
| | | CTGTGGAGTGGGAATCCAATGGCCAGCCTGAGAACAATTACAAGACCA |
| | | CACCCCCTGTGCTGGACTCTGATGGCAGTTTCTTTCTGTATAGTAAGCTG |
| | | ACCGTCGATAAATCACGATGGCAGCAGGGGAACGTGTTCAGCTGTTCA |
| | | GTGATGCACGAAGCCCTGCACAACCATTACACCCAGAAGAGCCTGAGC |
| | | CTGTCTCCCGGC |
| 116 | 14399 | EVKLVESEGGLVQPGSSMKLSCTASGFTFSDYYMAWVRQVPEKGLEWVA |
| | | HINYDGSGTYYLDSLKGRFIISRDNAKNILYLQMSSLKSEDTATYYCARDC |
| | | YGSSSYAVDYWGQGTSVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKDT |
| | | LMISRTPEVTCVVVSVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| 117 | 14399 | GAGGTGAAGCTGGTGGAGAGCGAGGGAGGACTGGTGCAGCCAGGCAG |
| | | CTCCATGAAGCTGAGCTGCACCGCCTCCGGCTTCACATTTTCCGACTACT |
| | | ATATGGCATGGGTGCGGCAGGTGCCAGAGAAGGGACTGGAGTGGGTGG |
| | | CCCACATCAACTACGACGGCTCTGGCACATACTATCTGGATAGCCTGAA |
| | | GGGCCGGTTTATCATCTCCAGAGACAACGCCAAGAATATCCTGTACCTG |
| | | CAGATGTCTAGCCTGAAGTCTGAGGACACCGCCACATACTATTGCGCCA |
| | | GGGATTGTTATGGCTCCTCTAGCTACGCCGTGGATTATTGGGGCCAGGG |
| | | CACCTCTGTGACAGTGTCCTCTGCTAGCACAAAGGGCCCCTCCGTGTTT |
| | | CCTCTGGCCCCATCCTCTAAGTCCACCTCTGGAGGAACAGCCGCCCTGG |
| | | GCTGTCTGGTGAAGGATTACTTCCCTGAGCCAGTGACCGTGTCCTGGAA |
| | | CTCTGGGGCCCTGACCAGCGGAGTGCACACATTTCCCGCCGTGCTGCAG |
| | | AGCTCCGGACTGTACTCCCTGTCTAGCGTGGTGACCGTGCCTTCCTCTAG |
| | | CCTGGGCACCCAGACATATATCTGCAACGTGAATCACAAGCCTTCCAAT |
| | | ACAAAGGTCGACAAGAAGGTGGAGCCAAAGTCTTGTGATAAGACCCAC |
| | | ACATGCCCACCTTGTCCGGCGCCAGAGGCCGCCGGAGGACCAAGCGTG |
| | | TTCCTGTTTCCACCCAAGCCCAAGGACACCCTGATGATCTCCCGGACCC |
| | | CAGAGGTGACATGCGTGGTGGTGAGCGTGTCCCACGAGGACCCCGAGG |
| | | TGAAGTTTAACTGGTACGTGGATGGCGTGGAGGTGCACAATGCCAAGA |
| | | CAAAGCCCCGGGAGGAGCAGTACAATTCTACCTATAGAGTGGTGAGCG |
| | | TGCTGACAGTGCTGCACCAGGATTGGCTGAACGGCAAGGAGTATAAGT |
| | | GTAAGGTGAGCAATAAGGCCCTGCCAGCCCCCATCGAGAAGACCATCT |
| | | CCAAGGCCAAGGGCCAGCCTCGCGAACCACAGGTGTACACTCTGCCTCC |
| | | ATCTCGGGACGAGCTGACTAAGAACCAGGTCAGTCTGACCTGTCTGGTG |
| | | AAAGGATTCTATCCCAGCGATATCGCTGTGGAGTGGGAATCCAATGGCC |
| | | AGCCTGAGAACAATTACAAGACCACACCCCCTGTGCTGGACTCTGATGG |
| | | CAGTTTCTTTCTGTATAGTAAGCTGACCGTCGATAAATCACGATGGCAG |
| | | CAGGGGAACGTGTTCAGCTGTTCAGTGATGCACGAAGCCCTGCACAACC |
| | | ATTACACCCAGAAGAGCCTGAGCCTGTCTCCCGGC |
| 118 | 14400 | QVQLQESGPGLVQPTQSLSITCTVSGFSLISYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 119 | 14400 | CAGGTGCAGCTGCAGGAGAGCGGACCAGGACTGGTGCAGCCTACACAG |
| | | TCTCTGAGCATCACCTGCACAGTGTCTGGCTTCAGCCTGATCTCCTACGG |
| | | AGTGCACTGGGTGAGGCAGTCCCCTGGCAAGGGACTGGAGTGGCTGGG |
| | | CGTGATCTGGTCTGGCGGCAGCACCGACTATAACGCCGCCTTTATCTCC |
| | | CGCCTGTCCATCTCTAAGGATAACAGCAAGTCCCAGGTGTTCTTTAAGA |
| | | TGAACAGCCTGCAGGCCGACGATACAGCCATCTACTATTGTGCCCGGAA |
| | | TCCCCTGACCGCCACAGTGATGGACTACTGGGGCCAGGGCACCTCTGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTGTACACTCTGCCTCCATCTCGGGAC |
| | | GAGCTGACTAAGAACCAGGTCAGTCTGACCTGTCTGGTGAAAGGATTCT |
| | | ATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 120 | 14401 | QVQLKESGPGLVAPSQSLSITCTVSGFSLTSYAISWVRQPPGKGLEWLGVIW |
| | | PGGGTNYNSALKSRLSISKDNSKSQVFLKMNSLQTDDTARYYCARGTGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 121 | 14401 | CAGGTGCAGCTGAAGGAGTCTGGACCAGGACTGGTGGCCCCCTCTCAG |
| | | AGCCTGTCCATCACCTGCACAGTGAGCGGCTTTTCCCTGACCTCTTACGC |
| | | AATCTCCTGGGTGCGGCAGCCACCTGGCAAGGGACTGGAGTGGCTGGG |
| | | CGTGATCTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAATAGCCTGCAGACCGACGATACAGCCCGGTACTATTGTGCCAGA |
| | | GGCACCGGCACATGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTGTACACTCTGCCTCCATCTCGGGAC |
| | | GAGCTGACTAAGAACCAGGTCAGTCTGACCTGTCTGGTGAAAGGATTCT |
| | | ATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 122 | 14402 | DVVMTQTPLSLPVSLGDQASISCRSSQSIVHSNGNTYLEWYLQKPGQSPKL |
| | | LIYKVSNRFSGVPDRFSARGSGTDFSLKISRVEAEDLGVYYCFQGSHVPYTF |
| | | GGGTKLEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQ |
| | | GLSSPVTKSFNRGEC |
| 123 | 14402 | GACGTGGTCATGACCCAGACACCCCTGTCCCTGCCCGTGAGCCTGGGCG |
| | | ACCAGGCCTCTATCAGCTGCAGGAGCTCCCAGAGCATCGTGCACTCCAA |
| | | CGGCAATACCTACCTGGAGTGGTATCTGCAGAAGCCAGGCCAGTCTCCC |
| | | AAGCTGCTGATCTACAAGGTGTCTAACCGGTTCAGCGGAGTGCCAGACC |
| | | GGTTTTCCGCCAGAGGCTCTGGCACAGACTTCAGCCTGAAGATCTCCAG |
| | | AGTGGAGGCCGAGGACCTGGGCGTGTACTATTGTTTCCAGGGCTCCCAC |
| | | GTGCCTTATACCTTTGGCGGCGGCACAAAGCTGGAGATCAAGAGAACA |
| | | GTGGCGGCGCCCAGTGTCTTCATTTTTCCCCCTAGCGACGAACAGCTGA |
| | | AGTCTGGGACAGCCAGTGTGGTCTGTCTGCTGAACAACTTCTACCCTAG |
| | | AGAGGCTAAAGTGCAGTGGAAGGTCGATAACGCACTGCAGTCCGGAAA |
| | | TTCTCAGGAGAGTGTGACTGAACAGGACTCAAAAGATAGCACCTATTCC |
| | | CTGTCAAGCACACTGACTCTGAGCAAGGCCGACTACGAGAAGCATAAA |
| | | GTGTATGCTTGTGAAGTCACCCACCAGGGGCTGAGTTCACCAGTCACAA |
| | | AATCATTCAACAGAGGGGAGTGC |
| 124 | 14404 | DIVMTQSHKFMSTSVGDRVSITCKASQDVGTAVGWYQQKLGQSPKLLIYW |
| | | ASTRRTGVPDRFTGSGSGTDFTLTISNVQSEDLADYFCQQYSSYPLTFGAGT |
| | | KLELKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |
| 125 | 14404 | GACATCGTGATGACCCAGAGCCACAAGTTTATGTCTACAAGCGTGGGCG |
| | | ACCGCGTGTCTATCACCTGCAAGGCCAGCCAGGATGTGGGAACAGCCG |
| | | TGGGCTGGTACCAGCAGAAGCTGGGCCAGTCCCCCAAGCTGCTGATCTA |
| | | TTGGGCCTCTACCCGGAGAACAGGAGTGCCTGACCGGTTCACCGGCTCC |
| | | GGCTCTGGCACAGACTTCACCCTGACAATCAGCAACGTGCAGTCCGAGG |
| | | ACCTGGCCGATTACTTCTGTCAGCAGTACAGCTCCTATCCCCTGACCTTC |
| | | GGGGCAGGGACAAAGCTGGAGCTGAAGAGGACAGTGGCGGCGCCCAG |
| | | TGTCTTCATTTTTCCCCCTAGCGACGAACAGCTGAAGTCTGGGACAGCC |
| | | AGTGTGGTCTGTCTGCTGAACAACTTCTACCCTAGAGAGGCTAAAGTGC |
| | | AGTGGAAGATCGATAACGCACTGCAGTCCGGAAATTCTCAGGAGAGTG |
| | | TGACTGAACAGGACTCAAAAGATAGCACCTATTCCCTGTCAAGCACACT |
| | | GACTCTGAGCAAGGCCGACTACGAGAAGCATAAAGTGTATGCTTGTGA |
| | | AGTCACCCACCAGGGGCTGAGTTCACCAGTCACAAAATCATTCAACAG |
| | | AGGGGAGTGC |
| 126 | 14405 | DIVMTQSQKFMSTSVGDRVSVTCKASQNVGTNVAWYQQKPGQSRTVLIYS |
| | | ASYRHSGVPDRFTGSGSGTDFTLTISNVQSEDLAEYFCQQYNSYPLTFGTGT |
| | | KLELKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |
| 127 | 14405 | GACATCGTGATGACCCAGAGCCAGAAGTTTATGAGCACATCCGTGGGC |
| | | GATCGCGTGTCTGTGACCTGCAAGGCCAGCCAGAACGTGGGCACAAAT |
| | | GTGGCCTGGTACCAGCAGAAGCCAGGCCAGTCCCGGACAGTGCTGATC |
| | | TACTCTGCCAGCTATCGGCACTCTGGAGTGCCAGACAGATTCACCGGCT |
| | | CCGGCTCTGGCACAGACTTCACCCTGACAATCAGCAACGTGCAGTCCGA |
| | | GGACCTGGCCGAGTATTTCTGTCAGCAGTACAATTCCTATCCCCTGACC |
| | | TTTGGCACCGGCACAAAGCTGGAGCTGAAGAGGACAGTGGCGGCGCCC |
| | | AGTGTCTTCATTTTTCCCCCTAGCGACGAACAGCTGAAGTCTGGGACAG |
| | | CCAGTGTGGTCTGTCTGCTGAACAACTTCTACCCTAGAGAGGCTAAAGT |
| | | GCAGTGGAAGGTCGATAACGCACTGCAGTCCGGAAATTCTCAGGAGAG |
| | | TGTGACTGAACAGGACTCAAAAGATAGCACCTATTCCCTGTCAAGCACA |
| | | CTGACTCTGAGCAAGGCCGACTACGAGAAGCATAAAGTGTATGCTTGTG |
| | | AAGTCACCCACCAGGGGCTGAGTTCACCAGTCACAAAATCATTCAACA |
| | | GAGGGGAGTGC |
| 128 | 14406 | DIVMTQAAFSNPVTLGTSASISCRSSKSLLHSYGITYLYWYLQKPGQSPQLLI |
| | | YQMSNLASGVPDRFSSSGSGTDFTLRISRVEAEDVGVYYCAQNLELPLTFG |
| | | AGTKLELKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| 129 | 14406 | GACATCGTGATGACACAGGCCGCCTTTTCTAACCCCGTGACCCTGGGCA |
| | | CATCTGCCAGCATCTCCTGCCGGAGCTCCAAGTCTCTGCTGCACAGCTA |
| | | TGGCATCACCTACCTGTATTGGTACCTGCAGAAGCCCGGCCAGTCCCCT |
| | | CAGCTGCTGATCTACCAGATGTCCAACCTGGCCTCTGGCGTGCCTGACA |
| | | GGTTCTCTAGCTCCGGCAGCGGAACCGACTTCACCCTGCGGATCTCCAG |
| | | AGTGGAGGCCGAGGATGTGGGCGTGTACTATTGTGCCCAGAATCTGGA |
| | | GCTGCCACTGACCTTCGGGGCAGGGACAAAGCTGGAGCTGAAGCGGAC |
| | | AGTGGCGGCGCCCAGTGTCTTCATTTTTCCCCCTAGCGACGAACAGCTG |
| | | AAGTCTGGGACAGCCAGTGTGGTCTGTCTGCTGAACAACTTCTACCCTA |
| | | GAGAGGCTAAAGTGCAGTGGAAGGTCGATAACGCACTGCAGTCCGGAA |
| | | ATTCTCAGGAGAGTGTGACTGAACAGGACTCAAAAGATAGCACCTATTC |
| | | CCTGTCAAGCACACTGACTCTGAGCAAGGCCGACTACGAGAAGCATAA |
| | | AGTGTATGCTTGTGAAGTCACCCACCAGGGGCTGAGTTCACCAGTCACA |
| | | AAATCATTCAACAGAGGGGAGTGC |
| 130 | 14407 | QIVLTQSPAIMSASPGEKVTMTCSASSSVSYMHWYQQKSGTSPKRWVYDT |
| | | SKLASGVPARFSGSGSGTSYSLTISSMEAEDVATYYCQQWSSNPPTFGGGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |
| 131 | 14407 | CAGATCGTGCTGACACAGAGCCCAGCCATCATGTCCGCCTCTCCAGGAG |
| | | AGAAGGTGACCATGACATGCAGCGCCAGCTCCAGCGTGAGCTACATGC |
| | | ACTGGTATCAGCAGAAGTCTGGCACCAGCCCTAAGCGGTGGGTGTACG |
| | | ACACATCCAAGCTGGCCTCTGGAGTGCCAGCCAGATTCAGCGGCTCCGG |
| | | CTCTGGCACCAGCTATTCCCTGACAATCAGCTCCATGGAGGCCGAGGAT |
| | | GTGGCCACCTACTATTGTCAGCAGTGGTCTAGCAACCCCCCTACCTTTG |
| | | GCGGCGGCACAAAGCTGGAGATCAAGCGGACAGTGGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTCAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 132 | 14408 | DVVMTQTPLSLPVSLGDQASISCRSSQSLVHSNGNTYLHWYLQKPGQSPKL |
| | | LIYKVSNRFFGVPDRFSGSGSGTDFTLKISRVEAEDLGVYFCSQTTYVPLTF |
| | | GAGTKLELKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQ |
| | | GLSSPVTKSFNRGEC |
| 133 | 14408 | GACGTGGTCATGACCCAGACACCCCTGTCCCTGCCCGTGAGCCTGGGCG |
| | | ACCAGGCCTCTATCAGCTGCCGGAGCTCCCAGAGCCTGGTGCACTCCAA |
| | | CGGCAATACATACCTGCACTGGTATCTGCAGAAGCCAGGCCAGTCTCCC |
| | | AAGCTGCTGATCTACAAGGTGAGCAACCGGTTCTTTGGCGTGCCCGACA |
| | | GATTCTCCGGCTCTGGCAGCGGAACCGACTTCACCCTGAAGATCTCCCG |
| | | GGTGGAGGCAGAGGACCTGGGCGTGTACTTCTGTTCTCAGACCACATAT |
| | | GTGCCTCTGACCTTCGGGGCAGGGACAAAGCTGGAGCTGAAGAGGACC |
| | | GTGGCGGCGCCCAGTGTCTTCATTTTTCCCCCTAGCGACGAACAGCTGA |
| | | AGTCTGGGACAGCCAGTGTGGTCTGTCTGCTGAACAACTTCTACCCTAG |
| | | AGAGGCTAAAGTGCAGTGGAAGGTCGATAACGCACTGCAGTCCGGAAA |
| | | TTCTCAGGAGAGTGTGACTGAACAGGACTCAAAAGATAGCACCTATTCC |
| | | CTGTCAAGCACACTGACTCTGAGCAAGGCCGACTACGAGAAGCATAAA |
| | | GTGTATGCTTGTGAAGTCACCCACCAGGGGCTGAGTTCACCAGTCACAA |
| | | AATCATTCAACAGAGGGGAGTGC |
| 134 | 14409 | NIVMTQSPKSMSMSVGERVTLSCKASENVGSYVSWYQQKPEKSPKLLIYG |
| | | ASNRYTGVPDRFTGSGSATDFTLTISSVQAEDLADYHCGQSYSYPLTFGAG |
| | | TKLELKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLSS |
| | | PVTKSFNRGEC |
| 135 | 14409 | AACATCGTGATGACCCAGTCCCCCAAGTCTATGAGCATGTCCGTGGGCG |
| | | AGCGCGTGACACTGTCTTGCAAGGCCAGCGAGAACGTGGGCAGCTACG |
| | | TGTCCTGGTATCAGCAGAAGCCCGAGAAGTCCCCTAAGCTGCTGATCTA |
| | | CGGGGCCAGCAATCGGTATACCGGCGTGCCTGACAGATTCACCGGCTCT |
| | | GGCAGCGCCACAGACTTCACCCTGACAATCAGCTCCGTGCAGGCAGAG |
| | | GACCTGGCAGATTACCACTGTGGCCAGTCCTACTCTTATCCACTGACCTT |
| | | CGGGGCAGGGACAAAGCTGGAGCTGAAGAGGACAGTGGCGGCGCCCA |
| | | GTGTCTTCATTTTTCCCCCTAGCGACGAACAGCTGAAGTCTGGGACAGC |
| | | CAGTGTGGTCTGTCTGCTGAACAACTTCTACCCTAGAGAGGCTAAAGTG |
| | | CAGTGGAAGGTCGATAACGCACTGCAGTCCGGAAATTCTCAGGAGAGT |
| | | GTGACTGAACAGGACTCAAAAGATAGCACCTATTCCCTGTCAAGCACAC |
| | | TGACTCTGAGCAAGGCCGACTACGAGAAGCATAAAGTGTATGCTTGTG |
| | | AAGTCACCCACCAGGGGCTGAGTTCACCAGTCACAAAATCATTCAACA |
| | | GAGGGGAGTGC |
| 136 | 14410 | DIVMTQSPSSLAVTAGEKVTMRCKSSQSLLWSVNQNNYLSWYQQKQGQP |
| | | PKLLIYGASIRESWVPDRFTGSGSGTDFTLTISNVHAEDLAVYYCQHNHGSF |
| | | LPYTFGGGTKLEIKRTVAAPSVFIFPPSDEQLKSGTASVVCLLNNFYPREAK |
| | | VQWKVDNALQSGNSQESVTEQESKDSTYSLSSTLTLSKADYEKHKVYACE |
| | | VTHQGLSSPVTKSFNRGEC |
| 137 | 14410 | GACATCGTGATGACACAGAGCCCAAGCTCCCTGGCCGTGACCGCCGGG |
| | | GAGAAGGTGACAATGCGGTGCAAGTCTAGCCAGAGCCTGCTGTGGTCC |
| | | GTGAACCAGAACAATTACCmTCCTGGTATCAGCAGAAGCAGGGCCAG |
| | | CCCCCTAAGCTGCTGATCTACGGGGCCAGCATCCGGGAGAGCTGGGTGC |
| | | CTGACAGATTCACCGGCTCCGGCTCTGGCACAGACTTCACCCTGACAAT |
| | | CTCCAACGTGCACGCCGAGGATCTGGCCGTGTACTATTGTCAGCACAAT |
| | | CACGGCTCTTTCCTGCCATATACCTTTGGCGGCGGCACAAAGCTGGAGA |
| | | TCAAGAGGACCGTGGCGGCGCCCAGTGTCTTCATTTTTCCCCCTAGCGA |
| | | CGAACAGCTGAAGTCTGGGACAGCCAGTGTGGTCTGTCTGCTGAACAAC |
| | | TTCTACCCTAGAGAGGCTAAAGTGCAGTGGAAGGTCGATAACGCACTG |
| | | CAGTCCGGAAATTCTCAGGAGAGTGTGACTGAACAGGACTCAAAAGAT |
| | | AGCACCTATTCCCTGTCAAGCACACTGACTCTGAGCAAGGCCGACTACG |
| | | AGAAGCATAAAGTGTATGCTTGTGAAGTCACCCACCAGGGGCTGAGTTC |
| | | ACCAGTCACAAAATCATTCAACAGAGGGGAGTGC |
| 138 | 14411 | DIQMTQTTSSLSASLGDRVTFSCSASQGISNYLNWYQQKPDGTVKLLIYYTS |
| | | SLHLGVPSRFSGSGSGTDYSLTISNLEPEDIATYYCQQYSKLPWTFGGGTKL |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 139 | 14411 | GACATCCAGATGACCCAGACCACAAGCTCCCTGTCCGCCTCTCTGGGCG |
| | | ATAGAGTGACCTTCAGCTGCTCCGCCTCTCAGGGCATCTCTAACTACCT |
| | | GAATTGGTATCAGCAGAAGCCTGACGGCACCGTGAAGCTGCTGATCTAC |
| | | TATACATCTAGCCTGCACCTGGGCGTGCCATCCAGGTTCAGCGGCTCCG |
| | | GCTCTGGAACCGACTACAGCCTGACAATCTCCAACCTGGAGCCCGAGG |
| | | ATATCGCCACCTACTATTGTCAGCAGTATAGCAAGCTGCCTTGGACCTT |
| | | TGGCGGCGGCACAAAGCTGGAGATCAAGCGCACAGTGGCGGCGCCCAG |
| | | TGTCTTCATTTTTCCCCCTAGCGACGAACAGCTGAAGTCTGGGACAGCC |
| | | AGTGTGGTCTGTCTGCTGAACAACTTCTACCCTAGAGAGGCTAAAGTGC |
| | | AGTGGAAGGTCGATAACGCACTGCAGTCCGGAAATTCTCAGGAGAGTG |
| | | TGACTGAACAGGACTCAAAAGATAGCACCTATTCCCTGTCAAGCACACT |
| | | GACTCTGAGCAAGGCCGACTACGAGAAGCATAAAGTGTATGCTTGTGA |
| | | AGTCACCCACCAGGGGCTGAGTTCACCAGTCACAAAATCATTCAACAG |
| | | AGGGGAGTGC |
| 140 | 14412 | NIVMTQSPKSMSMSVGERVTLSCKASDNVGISVSWYQQKPEQSPKLLIYGA |
| | | SNRYTGVPDRFTGTGSATDFTLTISSVQAEDLADYHCGQSYSYPFTFGSGTK |
| | | LEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| 141 | 14412 | AACATCGTGATGACCCAGTCCCCCAAGTCTATGAGCATGTCCGTGGGCG |
| | | AGCGCGTGACACTGAGCTGCAAGGCCTCCGACAACGTGGGCATCTCTGT |
| | | GAGCTGGTACCAGCAGAAGCCCGAGCAGTCTCCTAAGCTGCTGATCTAC |
| | | GGGGCCAGCAATCGGTATACCGGCGTGCCTGACAGATTCACCGGCACA |
| | | GGCTCCGCCACAGACTTCACCCTGACAATCAGCTCCGTGCAGGCAGAGG |
| | | ACCTGGCAGATTATCACTGTGGCCAGTCCTACTCTTATCCATTCACCTTT |
| | | GGCTCTGGCACAAAGCTGGAGATCAAGAGGACAGTGGCGGCGCCCAGT |
| | | GTCTTCATTTTTCCCCCTAGCGACGAACAGCTGAAGTCTGGGACAGCCA |
| | | GTGTGGTCTGTCTGCTGAACAACTTCTACCCTAGAGAGGCTAAAGTGCA |
| | | GTGGAAGGTCGATAACGCACTGCAGTCCGGAAATTCTCAGGAGAGTGT |
| | | GACTGAACAGGACTCAAAAGATAGCACCTATTCCCTGTCAAGCACACTG |
| | | ACTCTGAGCAAGGCCGACTACGAGAAGCATAAAGRTTATGCTTGTGAA |
| | | GTCACCCACCAGGGGCTGAGTTCACCAGTCACAAAATCATTCAACAGA |
| | | GGGGAGTGC |
| 142 | 18509 | QVQLKESGPGLVAPSQSLSITCTVSGFSLTTYAISWVRQPPGKGLEWLGIIW |
| | | PGGGTNYNSALKSRLSISKDNSRSQVFLKMNSLQTDDTARYYCARGAGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGQEQLVES |
| | | GGGLVQPEGSLTLTCTASKFSFSSLYYMCWVRQAPGKGLEWIACVYGGSS |
| | | GNTYYASWAKGRFTISKASSTTVTLQLTSLTAADTATYFCARFDVDGSGFN |
| | | LWGPGTLVTVSSGGGGSGGGGSGGGGSDIVMTQTPSSVSAAVGGTVTIKC |
| | | QASQTIGSSLAWYQQKPGQPPKLLIYRASTLASGVSSRFRGSGSGTEYTLTIS |
| | | DLECADAATYYCQWTDYGYIYIWAFGGGTEVVVK |
| 143 | 18509 | CAGGTGCAGGGAAGGAGTCCGGACCAGGACTGGTGGCCCCCTCTCAG |
| | | AGCCTGTCCATCACCTGCACAGTGAGCGGCTTTTCCCTGACCACATACG |
| | | CAATCTCTTGGGTGCGGCAGCCACCTGGCAAGGGACTGGAGTGGCTGG |
| | | GAATCATCTGGCCAGGAGGAGGCACAAACTATAATTCTGCCCTGAAGA |
| | | GCAGGCTGTCTATCAGCAAGGACAACTCCCGCTCTCAGGTGTTCCTGAA |
| | | GATGAACAGCCTGCAGACCGACGATACAGCAAGGTACTATTGTGCCCG |
| | | GGGGGCAGGGACCTGGTACTTTGACGTGTGGGGGGCAGGGACCACAGT |
| | | GACAGTGAGCTCCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAGCCCCAGGTGTACGTGTATCCCCCTAGCAGAG |
| | | ACGAGCTGACAAAGAACCAGGTGTCCCTGACCTGCCTGGTGAAGGGCT |
| | | TCTATCCCTCTGATATCGCCGTGGAGTGGGAGAGCAATGGCCAGCCTGA |
| | | GAACAATTACAAGACCACACCACCCGTGCTGGACAGCGATGGCTCCTTC |
| | | GCCCTGGTGAGCAAGCTGACAGTGGACAAGTCCAGGTGGCAGCAGGGC |
| | | AACGTGTTTTCTTGTAGCGTGATGCACGAGGCCCTGCACAATCACTATA |
| | | CCCAGAAGTCCCTGTCTCTGAGCCCAGGAGGAGGAGGAGGACAGGAGC |
| | | AGCTGGTGGAGTCTGGCGGCGGCCTGGTGCAGCCAGAGGGCTCCCTGA |
| | | CCCTGACATGCACCGCCTCTAAGTTCAGCTTTAGCTCCCTGTACTATATG |
| | | TGCTGGGTGAGGCAGGCCCCCGGCAAGGGACTGGAGTGGATCGCCTGC |
| | | GTGTATGGCGGCTCTAGCGGCAACACCTACTATGCCTCCTGGGCCAAGG |
| | | GCCGCTTCACAATCTCTAAGGCCTCCTCTACCACAGTGACCCTGCAGCT |
| | | GACAAGCCTGACCGCCGCCGACACAGCCACCTACTTCTGTGCCCGGTTT |
| | | GACGTGGATGGCTCCGGCTTTAATCTGTGGGGCCCTGGCACACTGGTGA |
| | | CCGTGAGCTCCGGAGGAGGAGGCAGCGGAGGAGGAGGCTCCGGCGGC |
| | | GGCGGCTCTGATATCGTGATGACACAGACCCCATCTAGCGTGAGCGCCG |
| | | CCGTGGGAGGCACAGTGACCATCAAGTGCCAGGCCTCCCAGACCATCG |
| | | GCTCCTCTCTGGCCTGGTATCAGCAGAAGCCTGGCCAGCCTCCAAAGCT |
| | | GCTGATCTACAGAGCCTCCACACTGGCCTCTGGCGTGAGCTCCCGGTTC |
| | | AGAGGCTCCGGCTCTGGAACCGAGTACACACTGACCATCAGCGACCTG |
| | | GAGTGCGCAGATGCAGCAACATACTATTGTCAGTGGACCGATTACGGCT |
| | | ATATCTACATCTGGGCCTTTGGCGGAGGAACCGAGGTGGTGGTGAAG |
| 144 | 14414 | DVLMTQTPLSLPVSLGDQASISCRSSQSIVHSNGNTYLEWYLQKPGQSPKLL |
| | | IYKVSNRFSGVPDRFSGSGSGTDFTLKISRVEAEDLGVYYCFQGSHVPWTF |
| | | GGGTKLEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQ |
| | | GLSSPVTKSFNRGEC |
| 145 | 14414 | GACGTGCTGATGACCCAGACACCCCTGTCCCTGCCCGTGAGCCTGGGCG |
| | | ACCAGGCCTCTATCAGCTGCCGGAGCTCCCAGAGCATCGTGCACTCCAA |
| | | CGGCAATACATACCTGGAGTGGTATCTGCAGAAGCCAGGCCAGTCCCCC |
| | | AAGCTGCTGATCTACAAGGTGTCTAACCGGTTCAGCGGCGTGCCCGACA |
| | | GATTTTCCGGCTCTGGCAGCGGAACCGACTTCACCCTGAAGATCTCCCG |
| | | GGTGGAGGCAGAGGACCTGGGCGTGTACTATTGTTTCCAGGGCTCTCAC |
| | | GTGCCTTGGACCTTTGGCGGCGGCACAAAGCTGGAGATCAAGAGGACC |
| | | GTGGCGGCGCCCAGTGTCTTCATTTTTCCCCCTAGCGACGAACAGCTGA |
| | | AGTCTGGGACAGCCAGTGTGGTCTGTCTGCTGAACAACTTCTACCCTAG |
| | | AGAGGCTAAAGTGCAGTGGAAGGTCGATAACGCACTGCAGTCCGGAAA |
| | | TTCTCAGGAGAGTGTGACTGAACAGGACTCAAAAGATAGCACCTATTCC |
| | | CTGTCAAGCACACTGACTCTGAGCAAGGCCGACTACGAGAAGCATAAA |
| | | GTGTATGCTTGTGAAGTCACCCACCAGGGGCTGAGTTCACCAGTCACAA |
| | | AATCATTCAACAGAGGGGAGTGC |
| 146 | 14415 | DIVMTQSQKFMSTSVGDRVSVTCKASQNVGTNVAWYQQKPGQSPKALIYS |
| | | ASYRDSGVPDRFTGSGSGTDFTLTISNVQSEDLAEYLCQQYNSYPLTFGAG |
| | | TKLELKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLSS |
| | | PVTKSFNRGEC |
| 147 | 14415 | GACATCGTGATGACCCAGAGCCAGAAGTTTATGAGCACATCCGTGGGC |
| | | GATCGCGTGTCTGTGACCTGCAAGGCCAGCCAGAACGTGGGCACAAAT |
| | | GTGGCCTGGTACCAGCAGAAGCCCGGCCAGTCCCCTAAGGCCCTGATCT |
| | | ACTCTGCCAGCTATCGGGACTCTGGCGTGCCTGATAGATTCACCGGCTC |
| | | CGGCTCTGGCACAGACTTTACCCTGACAATCAGCAACGTGCAGTCCGAG |
| | | GATCTGGCCGAGTACCTGTGCCAGCAGTACAATTCCTATCCCCTGACCT |
| | | TCGGGGCAGGGACAAAGCTGGAGCTGAAGAGGACAGTGGCGGCGCCCA |
| | | GTGTCTTCATTTTTCCCCCTAGCGACGAACAGCTGAAGTCTGGGACAGC |
| | | CAGTGTGGTCTGTCTGCTGAACAACTTCTACCCTAGAGAGGCTAAAGTG |
| | | CAGTGGAAGGTCGATAACGCACTGCAGTCCGGAAATTCTCAGGAGAGT |
| | | GTGACTGAACAGGACTCAAAAGATAGCACCTATTCCCTGTCAAGCACAC |
| | | TGACTCTGAGCAAGGCCGACTACGAGAAGCATAAAGTGTATGCTTGTG |
| | | AAGTCACCCACCAGGGGCTGAGTTCACCAGTCACAAAATCATTCAACA |
| | | GAGGGGAGTGC |
| 148 | 14416 | DIQMTQTTSSLSASLGDRVTISCSASQGISNYLNWYQQKPDGTVKLLIYYTS |
| | | SLHSGVPSRFSGSGSGTDYSLTISNLEPEDIATYYCQQYSDLPWTFGGGTKL |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 149 | 14416 | GACATCCAGATGACCCAGACCACAAGCTCCCTGTCCGCCTCTCTGGGCG |
| | | ATAGAGTGACAATCAGCTGCTCCGCCTCTCAGGGCATCTCCAACTACCT |
| | | GAATTGGTATCAGCAGAAGCCTGACGGCACCGTGAAGCTGCTGATCTAC |
| | | TATACATCTAGCCTGCACAGCGGAGTGCCATCCAGGTTCAGCGGCTCCG |
| | | GCTCTGGAACCGATTACTCTCTGACAATCAGCAACCTGGAGCCCGAGGA |
| | | CATCGCCACCTACTATTGTCAGCAGTATTCTGATCTGCCTTGGACCTTTG |
| | | GCGGCGGCACAAAGCTGGAGATCAAGCGCACAGTGGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 150 | 18519 | QVQLKESGPGLVAPSQSLSITCTVSGFSLTTYAISWVRQPPGKGLEWLGIIW |
| | | PGGGTNYNSALKSRLSISKDNSRSQVFLKMNSLQTDDTARYYCARGAGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPFKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVLPPSRD |
| | | ELTKNQVSLLCLVKGFYPSDIAVEWESNGQPENNYLTWPPVLDSDGSFFLY |
| | | SKLTVDKSRWQQGNVFSCSVMHEALHNHYTQKSLSLSPG |
| 151 | 18519 | CAGGTGCAGCTGAAGGAGTCCGGACCAGGACTGGTGGCCCCCTCTCAG |
| | | AGCCTGTCCATCACCTGCACAGTGAGCGGCTTTTCCCTGACCACATACG |
| | | CAATCTCTTGGGTGCGGCAGCCACCTGGCAAGGGACTGGAGTGGCTGG |
| | | GAATCATCTGGCCAGGAGGAGGCACAAACTATAATTCTGCCCTGAAGA |
| | | GCAGGCTGTCTATCAGCAAGGACAACTCCCGCTCTCAGGTGTTCCTGAA |
| | | GATGAACAGCCTGCAGACCGACGATACAGCAAGGTACTATTGTGCCCG |
| | | GGGGGCAGGGACCTGGTACTTTGACGTGTGGGGGGCAGGGACCACAGT |
| | | GACAGTGAGCTCCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAACCACAGGTCTACGTGCTGCCCCCTAGCCGCGA |
| | | CGAACTGACTAAAAATCAGGTCTCTCTGCTGTGTCTGGTCAAAGGATTC |
| | | TACCCTTCCGACATCGCCGTGGAGTGGGAAAGTAACGGCCAGCCCGAG |
| | | AACAATTACCTGACCTGGCCCCCTGTGCTGGACTCTGATGGGAGTTTCT |
| | | TTCTGTATTCAAAGCTGACAGTCGATAAAAGCCGGTGGCAGCAGGGCA |
| | | ATGTGTTCAGCTGCTCCGTCATGCACGAAGCACTGCACAACCATTACAC |
| | | TCAGAAGTCCCTGTCCCTGTCACCTGGC |
| 152 | 18520 | QVQLKQSGPGLVQPSQSLSITCTVSGFSLTSYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVLPPSRD |
| | | ELTKNQVSLLCLVKGFYPSDIAVEWESNGQPENNYLTWPPVLDSDGSFFLY |
| | | SKLTVDKSRWQQGNVFSCSVMHEALHNHYTQKSLSLSPG |
| 153 | 18520 | CAGGTGCAGCTGAAGCAGTCCGGACCAGGACTGGTGCAGCCTTCTCAG |
| | | AGCCTGTCCATCACCTGCACAGTGAGCGGCTTCTCCCTGACCTCTTACG |
| | | GCGTGCACTGGGTGAGGCAGTCTCCTGGCAAGGGACTGGAGTGGCTGG |
| | | GCGTGATCTGGAGCGGAGGCTCCACAGACTATAACGCCGCCTTTATCTC |
| | | TCGCCTGTCTATCAGCAAGGATAACTCCAAGTCTCAGGTGTTCTTTAAG |
| | | ATGAATAGCCTGCAGGCCGACGATACAGCCATCTACTATTGTGCCCGGA |
| | | ATCCCCTGACCGCCACAGTGATGGACTACTGGGGCCAGGGCACCAGCG |
| | | TGACAGTGAGCTCCGCTAGCACAAAGGGCCCCTCCGTGTTTCCTCTGGC |
| | | CCCATCCTCTAAGTCCACCTCTGGAGGAACAGCCGCCCTGGGCTGTCTG |
| | | GTGAAGGATTACTTCCCTGAGCCAGTGACCGTGTCCTGGAACTCTGGGG |
| | | CCCTGACCAGCGGAGTGCACACATTTCCCGCCGTGCTGCAGAGCTCCGG |
| | | ACTGTACTCCCTGTCTAGCGTGGTGACCGTGCCTTCCTCTAGCCTGGGCA |
| | | CCCAGACATATATCTGCAACGTGAATCACAAGCCTTCCAATACAAAGGT |
| | | CGACAAGAAGGTGGAGCCAAAGTCTTGTGATAAGACCCACACATGCCC |
| | | ACCTTGTCCGGCGCCAGAGGCCGCCGGAGGACCAAGCGTGTTCCTGTTT |
| | | CCACCCAAGCCCAAGGACACCCTGATGATCTCCCGGACCCCAGAGGTG |
| | | ACATGCGTGGTGGTGAGCGTGTCCCACGAGGACCCCGAGGTGAAGTTT |
| | | AACTGGTACGTGGATGGCGTGGAGGTGCACAATGCCAAGACAAAGCCC |
| | | CGGGAGGAGCAGTACAATTCTACCTATAGAGTGGTGAGCGTGCTGACA |
| | | GTGCTGCACCAGGATTGGCTGAACGGCAAGGAGTATAAGTGTAAGGTG |
| | | AGCAATAAGGCCCTGCCAGCCCCCATCGAGAAGACCATCTCCAAGGCC |
| | | AAGGGCCAGCCTCGCGAACCACAGGTCTACGTGCTGCCCCCTAGCCGCG |
| | | ACGAACTGACTAAAAATCAGGTCTCTCTGCTGTGTCTGGTCAAAGGATT |
| | | CTACCCTTCCGACATCGCCGTGGAGTGGGAAAGTAACGGCCAGCCCGA |
| | | GAACAATTACCTGACCTGGCCCCCTGTGCTGGACTCTGATGGGAGTTTC |
| | | TTTCTGTATTCAAAGCTGACAGTCGATAAAAGCCGGTGGCAGCAGGGCA |
| | | ATGTGTTCAGCTGCTCCGTCATGCACGAAGCACTGCACAACCATTACAC |
| | | TCAGAAGTCCCTGTCCCTGTCACCTGGC |
| 154 | 18521 | QVQLKESGPGLVAPSQSLSITCTVSGFSLTSYAINWVRQPPGKGLEWLGVL |
| | | WPGGGTNYNSALKSRLSISKDNSKSQVFLKMNSLQTDDTARYYCARGSGT |
| | | WYFDVWGAGTTVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVLPPSRD |
| | | ELTKNQVSLLCLVKGFYPSDIAVEWESNGQPENNYLTWPPVLDSDGSFFLY |
| | | SKLTVDKSRWQQGNVFSCSVMHEALHNHYTQKSLSLSPG |
| 155 | 18521 | CAGGTGCAGCTGAAGGAGAGCGGACCAGGACTGGTGGCCCCCTCTCAG |
| | | AGCCTGTCCATCACCTGCACAGTGAGCGGCTTTTCCCTGACCTCYTACGC |
| | | CATCAACTGGGTGCGGCAGCCACCTGGCAAGGGACTGGAGTGGCTGGG |
| | | CGTGCTGTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAACAGCCTGCAGACCGACGATACAGCCCGGTACTATTGTGCCAGA |
| | | GGCTCCGGCACCTGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTCTACGTGCTGCCCCCTAGCCGCGAC |
| | | GAACTGACTAAAAATCAGGTCTCTCTGCTGTGTCTGGTCAAAGGATTCT |
| | | ACCCTTCCGACATCGCCGTGGAGTGGGAAAGTAACGGCCAGCCCGAGA |
| | | ACAATTACCTGACCTGGCCCCCTGTGCTGGACTCTGATGGGAGTTTCTTT |
| | | CTGTATTCAAAGCTGACAGTCGATAAAAGCCGGTGGCAGCAGGGCAAT |
| | | GTGTTCAGCTGCTCCGTCATGCACGAAGCACTGCACAACCATTACACTC |
| | | AGAAGTCCCTGTCCCTGTCACCTGGC |
| 156 | 18522 | QVQLQESGPGLVQPTQSLSITCTVSGFSLISYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVLPPSRD |
| | | ELTKNQVSLLCLVKGFYPSDIAVEWESNGQPENNYLTWPPVLDSDGSFFLY |
| | | SKLTVDKSRWQQGNVFSCSVMHEALHNHYTQKSLSLSPG |
| 157 | 18522 | CAGGTGCAGCTGCAGGAGAGCGGACCAGGACTGGTGCAGCCTACACAG |
| | | TCTCTGAGCATCACCTGCACAGTGTCTGGCTTCAGCCTGATCTCCTACGG |
| | | AGTGCACTGGGTGAGGCAGTCCCCTGGCAAGGGACTGGAGTGGCTGGG |
| | | CGTGATCTGGTCTGGCGGCAGCACCGACTATAACGCCGCCTTTATCTCC |
| | | CGCCTGTCCATCTCTAAGGATAACAGCAAGTCCCAGGTGTTCTTTAAGA |
| | | TGAACAGCCTGCAGGCCGACGATACAGCCATCTACTATTGTGCCCGGAA |
| | | TCCCCTGACCGCCACAGTGATGGACTACTGGGGCCAGGGCACCTCTGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCACCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTCTACGTGCTGCCCCCTAGCCGCGAC |
| | | GAACTGACTAAAAATCAGGTCTCTCTGCTGTGTCTGGTCAAAGGATTCT |
| | | ACCCTTCCGACATCGCCGTGGAGTGGGAAAGTAACGGCCAGCCCGAGA |
| | | ACAATTACCTGACCTGGCCCCCTGTGCTGGACTCTGATGGGAGTTTCTTT |
| | | CTGTATTCAAAGCTGACAGTCGATAAAAGCCGGTGGCAGCAGGGCAAT |
| | | GTGTTCAGCTGCTCCGTCATGCACGAAGCACTGCACAACCATTACACTC |
| | | AGAAGTCCCTGTCCCTGTCACCTGGC |
| 158 | 18523 | QVQLKESGPGLVAPSQSLSITCTVSGFSLTSYAISWVRQPPGKGLEWLGVIW |
| | | PGGGTNYNSALKSRLSISKDNSKSQVFLKMNSLQTDDTARYYCARGTGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVLPPSRD |
| | | ELTKNQVSLLCLVKGFYPSDIAVEWESNGQPENNYLTWPPVLDSDGSFFLY |
| | | SKLTVDKSRWQQGNVFSCSVMHEALHNHYTQKSLSLSPG |
| 159 | 18523 | CAGGTGCAGCTGAAGGAGTCTGGACCAGGACTGGTGGCCCCCTCTCAG |
| | | AGCCTGTCCATCACCTGCACAGTGAGCGGCTTTTCCCTGACCTCTTACGC |
| | | AATCTCCTGGGTGCGGCAGCCACCTGGCAAGGGACTGGAGTGGCTGGG |
| | | CGTGATCTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAATAGCCTGCAGACCGACGATACAGCCCGGTACTATTGTGCCAGA |
| | | GGCACCGGCACATGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTCTACGTGCTGCCCCCTAGCCGCGAC |
| | | GAACTGACTAAAAATCAGGTCTCTCTGCTGTGTCTGGTCAAAGGATTCT |
| | | ACCCTTCCGACATCGCCGTGGAGTGGGAAAGTAACGGCCAGCCCGAGA |
| | | ACAATTACCTGACCTGGCCCCCTGTGCTGGACTCTGATGGGAGTTTCTTT |
| | | CTGTATTCAAAGCTGACAGTCGATAAAAGCCGGTGGCAGCAGGGCAAT |
| | | GTGTTCAGCTGCTCCGTCATGCACGAAGCACTGCACAACCATTACACTC |
| | | AGAAGTCCCTGTCCCTGTCACCTGGC |
| 160 | 18524 | QVQLKESGPGLVAPSQSLSITCTVSGFSLTTYAISWVRQPPGKGLEWLGIIW |
| | | PGGGTNYNSALKSRLSISKDNSRSQVFLKMNSLQTDDTARYYCARGAGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGQVQLVQ |
| | | SGAEVVKPGASVKISCKASGYTFTGYFMNWVKQSPGQSLEWIGRIHPYDG |
| | | DTFYNQKFQGKATLTVDKSSNTAHMELLSLTSEDFAVYYCTRYDGSRAMD |
| | | YWGQGTTVTVSSGGGGSGGGGSGGGGSDIVLTQSPLSLAVSLGQPAIISCK |
| | | ASQSVSFAGTSLMHWYHQKPGQQPRLLIYRASNLEAGVPDRFSGSGSKTDF |
| | | TLTISPVEAEDAATYYCQQSREYPYTFGGGTKLEIK |
| 161 | 18524 | CAGGTGCAGCTGAAGGAGTCCGGACCAGGACTGGTGGCCCCCTCTCAG |
| | | AGCCTGTCCATCACCTGCACAGTGAGCGGCTTTTCCCTGACCACATACG |
| | | CAATCTCTTGGGTGCGGCAGCCACCTGGCAAGGGACTGGAGTGGCTGG |
| | | GAATCATCTGGCCAGGAGGAGGCACAAACTATAATTCTGCCCTGAAGA |
| | | GCAGGCTGTCTATCAGCAAGGACAACTCCCGCTCTCAGGTGTTCCTGAA |
| | | GATGAACAGCCTGCAGACCGACGATACAGCAAGGTACTATTGTGCCCG |
| | | GGGGGCAGGGACCTGGTACTTTGACGTGTGGGGGGCAGGGACCACAGT |
| | | GACAGTGAGCTCCGCTAGCACAAAGGGCCCAAGCGTGTTTCCTCTGGCC |
| | | CCATCCTCTAAGAGCACCTCCGGAGGAACAGCCGCCCTGGGCTGTCTGG |
| | | TGAAGGACTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCTAATACAAAGGTC |
| | | GACAAGAAGGTGGAGCCAAAGAGCTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCAGGACCCCTGAGGTGA |
| | | CATGCGTGGTGGTGTCTGTGAGCCACGAGGACCCAGAGGTGAAGTTCA |
| | | ACTGGTACGTGGATGGCGTGGAGGTGCACAATGCCAAGACAAAGCCCA |
| | | GAGAGGAGCAGTACAATTCCACCTATCGCGTGGTGTCTGTGCTGACAGT |
| | | GCTGCACCAGGATTGGCTGAACGGCAAGGAGTATAAGTGCAAGGTGTC |
| | | CAATAAGGCCCTGCCAGCCCCCATCGAGAAGACCATCTCTAAGGCCAA |
| | | GGGCCAGCCTCGCGAACCTCAGGTGTACGTGTATCCTCCAAGCAGAGAC |
| | | GAGCTGACCAAGAACCAGGTGTCCCTGACATGTCTGGTGAAGGGCTTTT |
| | | ACCCCTCTGATATCGCCGTGGAGTGGGAGAGCAATGGCCAGCCTGAGA |
| | | ACAATTATAAGACCACACCCCCTGTGCTGGACAGCGATGGCTCCTTCGC |
| | | CCTGGTGAGCAAGCTGACCGTGGACAAGTCCAGGTGGCAGCAGGGCAA |
| | | CGTGTTTTCCTGCTCTGTGATGCACGAGGCCCTGCACAATCACTACACA |
| | | CAGAAGAGCCTGAGCTTAAGCCCAGGAGGAGGAGGAGGACAGGTGCA |
| | | GCTGGTGCAGAGCGGAGCCGAGGTGGTGAAGCCTGGGGCCAGCGTGAA |
| | | GATCAGCTGCAAGGCCTCCGGCTACACCTTCACAGGCTACTTCATGAAC |
| | | TGGGTGAAGCAGTCTCCTGGCCAGAGCCTGGAGTGGATCGGCAGAATC |
| | | CACCCATACGACGGCGATACCTTCTATAACCAGAAGTTTCAGGGCAAGG |
| | | CCACCCTGACAGTGGACAAGAGCTCCAATACCGCCCACATGGAGCTGCT |
| | | GTCCCTGACATCTGAGGATTTCGCCGTGTACTATTGCACCCGGTACGAC |
| | | GGCTCCAGAGCCATGGATTATTGGGGCCAGGGCACCACAGTGACAGTG |
| | | TCTAGCGGAGGAGGAGGCTCCGGAGGAGGAGGCTCTGGCGGCGGCGGC |
| | | AGCGACATCGTGCTGACCCAGAGCCCACTGTCCCTGGCCGTGTCCCTGG |
| | | GCCAGCCCGCCATCATCTCTTGTAAGGCCTCCCAGAGCGTGAGCTTCGC |
| | | CGGGACCAGCCTGATGCACTGGTACCACCAGAAGCCCGGCCAGCAGCC |
| | | CAGACTGCTGATCTATAGGGCCTCCAATCTGGAGGCCGGAGTGCCAGAC |
| | | CGGTTCTCCGGCTCTGGCAGCAAGACCGACTTCACCCTGACAATCAGCC |
| | | CTGTGGAGGCAGAGGATGCAGCAACATACTATTGTCAGCAGTCCAGGG |
| | | AGTACCCATATACCTTTGGCGGCGGCACAAAGCTGGAGATCAAG |
| 162 | 18552 | QVQLKESGPGLVAPSQSLSITCTVSGFSLTTYAISWVRQPPGKGLEWLGIIW |
| | | PGGGTNYNSALKSRLSISKDNSRSQVFLKMNSLQTDDTARYYCARGAGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPASVEAAVGGTVTIKCQASQSIYSSLAWYQQKPGQSPKLLIYDASHLASG |
| | | VPSRFSGSRYGTEFTLTISGVQCDDAATYYCQGGWYSSAATYVPNTFGGGT |
| | | EVVVKGGGGSGGGGSGGGGSQEQLVESGGGLVQPEGSLTLTCKASGFTIS |
| | | NNYYMCWVRQAPGKGLEWIACIYGGISGRTYYASWAKGRFTISKTSSTTV |
| | | TLQMTSLTAADTATYFCVRGYVGTSNLWGPGTLVTVSS |
| 163 | 18552 | CAGGTGCAGCTGAAGGAGTCCGGACCAGGACTGGTGGCCCCCTCTCAG |
| | | AGCCTGTCCATCACCTGCACAGTGAGCGGCTTTTCCCTGACCACATACG |
| | | CAATCTCTTGGGTGCGGCAGCCACCTGGCAAGGGACTGGAGTGGCTGG |
| | | GAATCATCTGGCCAGGAGGAGGCACAAACTATAATTCTGCCCTGAAGA |
| | | GCAGGCTGTCTATCAGCAAGGACAACTCCCGCTCTCAGGTGTTCCTGAA |
| | | GATGAACAGCCTGCAGACCGACGATACAGCAAGGTACTATTGTGCCCG |
| | | GGGGGCAGGGACCTGGTACTTTGACGTGTGGGGGGCAGGGACCACAGT |
| | | GACAGTGAGCTCCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAGCCACAGGTGTACGTGTATCCCCCTAGCAGGG |
| | | ACGAGCTGACAAAGAACCAGGTGTCCCTGACCTGCCTGGTGAAGGGCT |
| | | TCTACCCCTCCGATATCGCCGTGGAGTGGGAGTCTAATGGCCAGCCTGA |
| | | GAACAATTATAAGACCACACCACCCGTGCTGGACTCTGATGGCAGCTTC |
| | | GCCCTGGTGAGCAAGCTGACAGTGGACAAGTCCAGATGGCAGCAGGGC |
| | | AACGTGTTTTCTTGCAGCGTGATGCACGAGGCCCTGCACAATCACTACA |
| | | CCCAGAAGTCCCTGTCTCTGAGCCCAGGAGGAGGAGGAGGCGATATCG |
| | | TGATGACCCAGACACCCGCCTCCGTGGAGGCCGCCGTGGGAGGAACCG |
| | | TGACAATCAAGTGTCAGGCCTCCCAGTCTATCTACAGCTCCCTGGCCTG |
| | | GTATCAGCAGAAGCCTGGCCAGAGCCCAAAGCTGCTGATCTACGACGC |
| | | CTCCCACCTGGCCTCTGGAGTGCCAAGCCGGTTCAGCGGCTCCAGATAT |
| | | GGCACAGAGTTTACCCTGACAATCTCCGGAGTGCAGTGCGACGATGCA |
| | | GCAACCTACTATTGTCAGGGAGGATGGTACTCTAGCGCCGCCACCTATG |
| | | TGCCTAACACATTCGGCGGCGGCACCGAGGTGGTGGTGAAGGGAGGAG |
| | | GAGGCTCCGGCGGAGGAGGCTCTGGCGGCGGCGGCAGCCAGGAGCAGC |
| | | TGGTGGAGTCTGGAGGAGGACTGGTGCAGCCTGAGGGCAGCCTGACCC |
| | | TGACATGCAAGGCCTCCGGCTTTACCATCTCTAACAATTACTATATGTG |
| | | CTGGGTGCGGCAGGCCCCAGGCAAGGGACTGGAGTGGATCGCCTGCAT |
| | | CTACGGCGGCATCTCTGGCAGGACATACTATGCCAGCTGGGCCAAGGG |
| | | CCGCTTCACCATCTCCAAGACATCCTCTACCACAGTGACCCTGCAGATG |
| | | ACCTCTCTGACAGCCGCCGATACCGCCACATACTTTTGCGTGCGGGGCT |
| | | ATGTGGGCACCAGCAATCTGTGGGGCCCTGGCACCCTGGTGACAGTGA |
| | | GCTCC |
| 164 | 18553 | QVQLKQSGPGLVQPSQSLSITCTVSGFSLTSYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTYDKSRWQQGNVFSCSVMHEALHNHYTQKSLSLSPGGGGGDIVMTQ |
| | | TPASVEAAVGGTVTIKCQASQSIYSSLAWYQQKPGQSPKLLIYDASHLASG |
| | | VPSRFSGSRYGTEFTLTISGVQCDDAATYYCQGGWYSSAATYVPNTFGGGT |
| | | EVVVKGGGGSGGGGSGGGGSQEQLVESGGGLVQPEGSLTLTCKASGFTIS |
| | | NNYYMCWVRQAPGKGLEWIACIYGGISGRTYYASWAKGRFTISKTSSTTV |
| | | TLQMTSLTAADTATYFCVRGYVGTSNLWGPGTLVTVSS |
| 165 | 18553 | CAGGTGCAGCTGAAGCAGTCCGGACCAGGACTGGTGCAGCCTTCTCAG |
| | | AGCCTGTCCATCACCTGCACAGTGAGCGGCTTCTCCCTGACCTCTTACG |
| | | GCGTGCACTGGGTGAGGCAGTCTCCTGGCAAGGGACTGGAGTGGCTGG |
| | | GCGTGATCTGGAGCGGAGGCTCCACAGACTATAACGCCGCCTTTATCTC |
| | | TCGCCTGTCTATCAGCAAGGATAACTCCAAGTCTCAGGTGTTCTTTAAG |
| | | ATGAATAGCCTGCAGGCCGACGATACAGCCATCTACTATTGTGCCCGGA |
| | | ATCCCCTGACCGCCACAGTGATGGACTACTGGGGCCAGGGCACCAGCG |
| | | TGACAGTGAGCTCCGCTAGCACAAAGGGCCCCTCCGTGTTTCCTCTGGC |
| | | CCCATCCTCTAAGTCCACCTCTGGAGGAACAGCCGCCCTGGGCTGTCTG |
| | | GTGAAGGATTACTTCCCTGAGCCAGTGACCGTGTCCTGGAACTCTGGGG |
| | | CCCTGACCAGCGGAGTGCACACATTTCCCGCCGTGCTGCAGAGCTCCGG |
| | | ACTGTACTCCCTGTCTAGCGTGGTGACCGTGCCTTCCTCTAGCCTGGGCA |
| | | CCCAGACATATATCTGCAACGTGAATCACAAGCCTTCCAATACAAAGGT |
| | | CGACAAGAAGGTGGAGCCAAAGTCTTGTGATAAGACCCACACATGCCC |
| | | ACCTTGTCCGGCGCCAGAGGCCGCCGGAGGACCAAGCGTGTTCCTGTTT |
| | | CCACCCAAGCCCAAGGACACCCTGATGATCTCCCGGACCCCAGAGGTG |
| | | ACATGCGTGGTGGTGAGCGTGTCCCACGAGGACCCCGAGGTGAAGTTT |
| | | AACTGGTACGTGGATGGCGTGGAGGTGCACAATGCCAAGACAAAGCCC |
| | | CGGGAGGAGCAGTACAATTCTACCTATAGAGTGGTGAGCGTGCTGACA |
| | | GTGCTGCACCAGGATTGGCTGAACGGCAAGGAGTATAAGTGTAAGGTG |
| | | ACTCAATAAGGCCCTGCCAGCCCCCATCGAGAAGACCATCTCCAAGGCC |
| | | AAGGGCCAGCCTCGCGAGCCACAGGTGTACGTGTATCCCCCTAGCAGG |
| | | GACGAGCTGACAAAGAACCAGGTGTCCCTGACCTGCCTGGTGAAGGGC |
| | | TTCTACCCCTCCGATATCGCCGTGGAGTGGGAGTCTAATGGCCAGCCTG |
| | | AGAACAATTATAAGACCACACCACCCGTGCTGGACTCTGATGGCAGCTT |
| | | CGCCCTGGTGAGCAAGCTGACAGTGGACAAGTCCAGATGGCAGCAGGG |
| | | CAACGTGTTTTCTTGCAGCGTGATGCACGAGGCCCTGCACAATCACTAC |
| | | ACCCAGAAGTCCCTGTCTCTGAGCCCAGGAGGAGGAGGAGGCGATATC |
| | | GTGATGACCCAGACACCCGCCTCCGTGGAGGCCGCCGTGGGAGGAACC |
| | | GTGACAATCAAGTGTCAGGCCTCCCAGTCTATCTACAGCTCCCTGGCCT |
| | | GGTATCAGCAGAAGCCTGGCCAGAGCCCAAAGCTGCTGATCTACGACG |
| | | CCTCCCACCTGGCCTCTGGAGTGCCAAGCCGGTTCAGCGGCTCCAGATA |
| | | TGGCACAGAGTTTACCCTGACAATCTCCGGAGTGCAGTGCGACGATGCA |
| | | GCAACCTACTATTGTCAGGGAGGATGGTACTCTAGCGCCGCCACCTATG |
| | | TGCCTAACACATTCGGCGGCGGCACCGAGGTGGTGGTGAAGGGAGGAG |
| | | GAGGCTCCGGCGGAGGAGGCTCTGGCGGCGGCGGCAGCCAGGAGCAGC |
| | | TGGTGGAGTCTGGAGGAGGACTGGTGCAGCCTGAGGGCAGCCTGACCC |
| | | TGACATGCAAGGCCTCCGGCTTTACCATCTCTAACAATTACTATATGTG |
| | | CTGGGTGCGGCAGGCCCCAGGCAAGGGACTGGAGTGGATCGCCTGCAT |
| | | CTACGGCGGCATCTCTGGCAGGACATACTATGCCAGCTGGGCCAAGGG |
| | | CCGCTTCACCATCTCCAAGACATCCTCTACCACAGTGACCCTGCAGATG |
| | | ACCTCTCTGACAGCCGCCGATACCGCCACATACTTTTGCGTGCGGGGCT |
| | | ATGTGGGCACCAGCAATCTGTGGGGCCCTGGCACCCTGGTGACAGTGA |
| | | GCTCC |
| 166 | 18554 | QVQLKESGPGLVAPSQSLSITCTVSGFSLTSYAINWVRQPPGKGLEWLGVL |
| | | WPGGGYNYNSALKSRLSISKDNSKSQVFLKMNSLQTDDTARYYCARGSGT |
| | | WYFDVWGAGTTVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPASVEAAVGGTVTIKCQASQSIYSSLAWYQQKPGQSPKLLIYDASHLASG |
| | | VPSRFSGSRYGTEFTLTISGVQCDDAATYYCQGGWYSSAATYVPNTFGGGT |
| | | EVVVKGGGGSGGGGSGGGGSQEQLVESGGGLVQPEGSLTLTCKASGFTIS |
| | | NNYYMCWVRQAPGKGLEWIACIYGGISGRTYYASWAKGRFTISKTSSTTV |
| | | TLQMTSLTAADTATYFCVRGYVGTSNLWGPGTLVTVSS |
| 167 | 18554 | CAGGTGCAGCTGAAGGAGAGCGGACCAGGACTGGTGGCCCCCTCTCAG |
| | | AGCCTGTCCATCACCTGCACAGTGAGCGGCTTTTCCCTGACCTCTTACGC |
| | | CATCAACTGGGTGCGGCAGCCACCTGGCAAGGGACTGGAGTGGCTGGG |
| | | CGTGCTGTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAACAGCCTGCAGACCGACGATACAGCCCGGTACTATTGTGCCAGA |
| | | GGCTCCGGCACCTGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAGCCACAGGTGTACGTGTATCCCCCTAGCAGGGA |
| | | CGAGCTGACAAAGAACCAGGTGTCCCTGACCTGCCTGGTGAAGGGCTTC |
| | | TACCCCTCCGATATCGCCGTGGAGTGGGAGTCTAATGGCCAGCCTGAGA |
| | | ACAATTATAAGACCACACCACCCGTGCTGGACTCTGATGGCAGCTTCGC |
| | | CCTGGTGAGCAAGCTGACAGTGGACAAGTCCAGATGGCAGCAGGGCAA |
| | | CGTGTTTTCTTGCAGCGTGATGCACGAGGCCCTGCACAATCACTACACC |
| | | CAGAAGTCCCTGTCTCTGAGCCCAGGAGGAGGAGGAGGCGATATCGTG |
| | | ATGACCCAGACACCCGCCTCCGTGGAGGCCGCCGTGGGAGGAACCGTG |
| | | ACAATCAAGTGTCAGGCCTCCCAGTCTATCTACAGCTCCCTGGCCTGGT |
| | | ATCAGCAGAAGCCTGGCCAGAGCCCAAAGCTGCTGATCTACGACGCCT |
| | | CCCACCTGGCCTCTGGAGTGCCAAGCCGGETCAGCGGCTCCAGATATGG |
| | | CACAGAGTTTACCCTGACAATCTCCGGAGTGCAGTGCGACGATGCAGCA |
| | | ACCTACTATTGTCAGGGAGGATGGTACTCTAGCGCCGCCACCTATGTGC |
| | | CTAACACATTCGGCGGCGGCACCGAGGTGGTGGTGAAGGGAGGAGGAG |
| | | GCTCCGGCGGAGGAGGCTCTGGCGGCGGCGGCAGCCAGGAGCAGCTGG |
| | | TGGAGTCTGGAGGAGGACTGGTGCAGCCTGAGGGCAGCCTGACCCTGA |
| | | CATGCAAGGCCTCCGGCTTTACCATCTCTAACAATTACTATATGTGCTG |
| | | GGTGCGGCAGGCCCCAGGCAAGGGACTGGAGTGGATCGCCTGCATCTA |
| | | CGGCGGCATCTCTGGCAGGACATACTATGCCAGCTGGGCCAAGGGCCG |
| | | CTTCACCATCTCCAAGACATCCTCTACCACAGTGACCCTGCAGATGACC |
| | | TCTCTGACAGCCGCCGATACCGCCACATACTTTTGCGTGCGGGGCTATG |
| | | TGGGCACCAGCAATCTGTGGGGCCCTGGCACCCTGGTGACAGTGAGCTC |
| | | C |
| 168 | 18555 | QVQLQESGPGLVQPTQSLSITCTVSGFSLISYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPASVEAAVGGTVTIKCQASQSIYSSLAWYQQKPGQSPKLLIYDASHLASG |
| | | VPSRFSGSRYGTEFTLTISGVQCDDAATYYCQGGWYSSAATYVPNTFGGGT |
| | | EVVVKGGGGSGGGGSGGGGSQEQLVESGGGLVQPEGSLTLTCKASGFTIS |
| | | NNYYMCWVRQAPGKGLEWIACIYGGISGRTYYASWAKGRFTISKTSSTTV |
| | | TLQMTSLTAADTATYFCVRGYVGTSNLWGPGTLVTVSS |
| 169 | 18555 | CAGGTGCAGCTGCAGGAGAGCGGACCAGGACTGGTGCAGCCTACACAG |
| | | TCTCTGAGCATCACCTGCACAGTGTCTGGCTTCAGCCTGATCTCCTACGG |
| | | AGTGCACTGGGTGAGGCAGTCCCCTGGCAAGGGACTGGAGTGGCTGGG |
| | | CGTGATCTGGTCTGGCGGCAGCACCGACTATAACGCCGCCTTTATCTCC |
| | | CGCCTGTCCATCTCTAAGGATAACAGCAAGTCCCAGGTGTTCTTTAAGA |
| | | TGAACAGCCTGCAGGCCGACGATACAGCCATCTACTATTGTGCCCGGAA |
| | | TCCCCTGACCGCCACAGTGATGGACTACTGGGGCCAGGGCACCTCTGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAGCCACAGGTGTACGTGTATCCCCCTAGCAGGGA |
| | | CGAGCTGACAAAGAACCAGGTGTCCCTGACCTGCCTGGTGAAGGGCTTC |
| | | TACCCCTCCGATATCGCCGTGGAGTGGGAGTCTAATGGCCAGCCTGAGA |
| | | ACAATTATAAGACCACACCACCCGTGCTGGACTCTGATGGCAGCTTCGC |
| | | CCTGGTGAGCAAGCTGACAGTGGACAAGTCCAGATGGCAGCAGGGCAA |
| | | CGTGTTTTCTTGCAGCGTGATGCACGAGGCCCTGCACAATCACTACACC |
| | | CAGAAGTCCCTGTCTCTGAGCCCAGGAGGAGGAGGAGGCGATATCGTG |
| | | ATGACCCAGACACCCGCCTCCGTGGAGGCCGCCGTGGGAGGAACCGTG |
| | | ACAATCAAGTGTCAGGCCTCCCAGTCTATCTACAGCTCCCTGGCCTGGT |
| | | ATCAGCAGAAGCCTGGCCAGAGCCCAAAGCTGCTGATCTACGACGCCT |
| | | CCCACCTGGCCTCTGGAGTGCCAAGCCGGTTCAGCGGCTCCAGATATGG |
| | | CACAGAGTTTACCCTGACAATCTCCGGAGTGCAGTGCGACGATGCAGCA |
| | | ACCTACTATTGTCAGGGAGGATGGTACTCTAGCGCCGCCACCTATGTGC |
| | | CTAACACATTCGGCGGCGGCACCGAGGTGGTGGTGAAGGGAGGAGGAG |
| | | GCTCCGGCGGAGGAGGCTCTGGCGGCGGCGGCAGCCAGGAGCAGCTGG |
| | | TGGAGTCTGGAGGAGGACTGGTGCAGCCTGAGGGCAGCCTGACCCTGA |
| | | CATGCAAGGCCTCCGGCTTTACCATCTCTAACAATTACTATATGTGCTG |
| | | GGTGCGGCAGGCCCCAGGCAAGGGACTGGAGTGGATCGCCTGCATCTA |
| | | CGGCGGCATCTCTGGCAGGACATACTATGCCAGCTGGGCCAAGGGCCG |
| | | CTTCACCATCTCCAAGACATCCTCTACCACAGTGACCCTGCAGATGACC |
| | | TCTCTGACAGCCGCCGATACCGCCACATACTTTTGCGTGCGGGGCTATG |
| | | TGGGCACCAGCAATCTGTGGGGCCCTGGCACCCTGGTGACAGTGAGCTC |
| | | C |
| 170 | 5244 | DIQMTQSPSSLSASVGDRVTITCRASQDVNTAVAWYQQKPGKAPKLLIYSA |
| | | SFLYSGVPSRFSGSRSGTDFTLTISSLQPEDFATYYCQQHYTTPPTFGQGTKV |
| | | EIKGGSGGGSGGGSGGGSGGGSGEVQLVESGGGLVQPGGSLRLSCAASGF |
| | | NIKDTYIHWVRQAPGKGLEWVARIYPTNGYTRYADSVKGRFTISADTSKNT |
| | | AYLQMNSLRAEDTAVYYCSRWGGDGFYAMDYWGQGTLVTVSSAAEPKS |
| | | SDKTHTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKEY |
| | | KCKVSNKALPAPIEKTISKAKGQPREPQVYVLPPSRDELTKNQVSLLCLVK |
| | | GFYPSDIAVEWESNGQPENNYLTWPPVLDSDGSFELYSKLTVDKSRWQQG |
| | | NVFSCSVMHEALHNHYTQKSLSLSPG |
| 171 | 5244 | GACATTCAGATGACACAGAGCCCCAGCTCCCTGAGTGCTTCAGTCGGCG |
| | | ACAGGGTGACTATCACCTGCCGCGCATCCCAGGATGTCAACACCGCTGT |
| | | GGCATGGTACCAGCAGAAGCCTGGAAAAGCCCCAAAGCTGCTGATCTA |
| | | CAGCGCTTCCTTCCTGTATTCTGGCGTGCCAAGTCGGTTTTCTGGAAGTA |
| | | GATCAGGCACTGACTTCACACTGACTATCTCTAGTCTGCAGCCCGAAGA |
| | | TTTTGCCACCTACTATTGCCAGCAGCACTATACCACACCCCCTACATTCG |
| | | GACAGGGCACTAAAGTGGAGATTAAGGGCGGGTCAGGCGGAGGGAGC |
| | | GGAGGAGGGTCCGGAGGAGGGTCTGGAGGAGGGAGTGGAGAGGTCCA |
| | | GCTGGTGGAATCTGGAGGAGGACTGGTGCAGCCTGGAGGCTCACTGCG |
| | | ACTGAGCTGTGCCGCTTCCGGCTTTAACATCAAAGACACATACATTCAT |
| | | TGGGTCAGGCAGGCACCAGGGAAGGGACTGGAATGGGTGGCCCGCATC |
| | | TATCCCACAAATGGGTACACTCGATATGCCGACAGCGTGAAAGGACGG |
| | | TTTACCATTTCTGCTGATACCAGTAAGAACACAGCATACCTGCAGATGA |
| | | ACAGCCTGCGCGCAGAGGATACAGCCGTGTACTATTGCAGTCGATGGG |
| | | GGGGAGACGGCTTCTACGCCATGGATTATTGGGGCCAGGGGACTCTGGT |
| | | CACCGTGTCAAGCGCAGCCGAACCTAAATCCTCTGACAAGACCCACAC |
| | | ATGCCCACCCTGTCCTGCTCCAGAGCTGCTGGGAGGACCATCCGTGTTC |
| | | CTGTTTCCTCCAAAGCCTAAAGATACACTGATGATTAGCCGCACTCCCG |
| | | AAGTCACCTGTGTGGTCGTGGACGTGTCCCACGAGGACCCCGAAGTCAA |
| | | GTTCAACTGGTACGTGGACGGCGTCGAGGTGCATAATGCCAAGACTAA |
| | | ACCAAGAGAGGAACAGTACAATTCAACCTATAGGGTCGTGAGCGTCCT |
| | | GACAGTGCTGCATCAGGATTGGCTGAACGGCAAGGAGTATAAGTGCAA |
| | | AGTGTCTAACAAGGCCCTGCCCGCTCCTATCGAGAAGACTATTAGCAAG |
| | | GCAAAAGGGCAGCCACGGGAACCCCAGGTCTACGTGCTGCCCCCTAGC |
| | | AGAGACGAGCTGACCAAAAACCAGGTCTCCCTGCTGTGTCTGGTGAAG |
| | | GGCTTTTATCCTAGTGATATCGCTGTGGAGTGGGAATCAAATGGGCAGC |
| | | CAGAAAACAATTACCTGACATGGCCACCCGTGCTGGACAGCGATGGGT |
| | | CCTTCTTTCTGTATTCCAAACTGACTGTGGACAAGTCTAGATGGCAGCA |
| | | GGGAAACGTCTTCAGCTGTTCCGTGATGCACGAGGCCCTGCACAATCAT |
| | | TACACCCAGAAGTCTCTGAGTCTGTCACCCGGC |
| 172 | 18557 | QVQLKESGPGLVAPSQSLSITCTVSGFSLTTYAISWVRQPPGKGLEWLGIIW |
| | | PGGGTNYNSALKSRLSISKDNSRSQVFLKMNSLQTDDTARYYCARGAGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPSSVSAAVGGTVTIKCQASQTIGSSLAWYQQKPGQPPKLLIYRASTLASGV |
| | | SSRFRGSGSGTEYTLTISDLECADAATYYCQWTDYGYIYIWAFGGGTEVVV |
| | | KGGGGSGGGGSGGGGSQEQLVESGGGLVQPEGSLTLTCTASKFSFSSLYY |
| | | MCWVRQAPGKGLEWIACVYGGSSGNTYYASWAKGRFTISKASSITVTLQL |
| | | TSLTAADTATYFCARFDVDGSGFNLWGPGTLVTVSS |
| 173 | 18557 | CAGGTGCAGCTGAAGGAGTCCGGACCAGGACTGGTGGCCCCCTCTCAG |
| | | AGCCTGTCCATCACCTGCACAGTGAGCGGCTTTTCCCTGACCACATACG |
| | | CAATCTCTTGGGTGCGGCAGCCACCTGGCAAGGGACTGGAGTGGCTGG |
| | | GAATCATCTGGCCAGGAGGAGGCACAAACTATAATTCTGCCCTGAAGA |
| | | GCAGGCTGTCTATCAGCAAGGACAACTCCCGCTCTCAGGTGTTCCTGAA |
| | | GATGAACAGCCTGCAGACCGACGATACAGCAAGGTACTATTGTGCCCG |
| | | GGGGGCAGGGACCTGGTACTTTGACGTGTGGGGGGCAGGGACCACAGT |
| | | GACAGTGAGCTCCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAGCCCCAGGTGTACGTGTATCCCCCTAGCAGAG |
| | | ACGAGCTGACAAAGAACCAGGTGTCCCTGACCTGCCTGGTGAAGGGCT |
| | | TCTATCCCTCTGATATCGCCGTGGAGTGGGAGAGCAATGGCCAGCCTGA |
| | | GAACAATTACAAGACCACACCACCCGTGCTGGACAGCGATGGCTCCTTC |
| | | GCCCTGGTGAGCAAGCTGACAGTGGACAAGTCCAGGTGGCAGCAGGGC |
| | | AACGTGTTTTCTTGCAGCGTGATGCACGAGGCCCTGCACAATCACTATA |
| | | CCCAGAAGTCCCTGTCTCTGAGCCCAGGAGGAGGAGGAGGCGATATCG |
| | | TGATGACCCAGACACCAAGCTCCGTGAGCGCCGCCGTGGGAGGAACCG |
| | | TGACAATCAAGTGTCAGGCCTCCCAGACCATCGGCTCTAGCCTGGCCTG |
| | | GTATCAGCAGAAGCCTGGCCAGCCTCCAAAGCTGCTGATCTACAGAGCC |
| | | TCCACACTGGCCTCTGGCGTGTCCTCTCGGYTCAGAGGCTCCGGCTCTG |
| | | GCACCGAGTACACCCTGACAATCAGCGACCTGGAGTGCGCAGATGCAG |
| | | CAACATACTATTGTCAGTGGACCGACTACGGCTATATCTACATCTGGGC |
| | | CTTTGGCGGAGGAACCGAGGTGGTGGTGAAGGGAGGAGGAGGCAGCG |
| | | GCGGCGGAGGCTCCGGCGGCGGCGGCTCTCAGGAGCAGCTGGTGGAGT |
| | | CTGGAGGAGGACTGGTGCAGCCTGAGGGCTCCCTGACCCTGACATGCA |
| | | CCGCCTCTAAGTTCAGCTTTAGCTCCCTGTACTATATGTGCTGGGTGAGG |
| | | CAGGCCCCAGGCAAGGGACTGGAGTGGATCGCCTGCGTGTATGGCGGC |
| | | TCTAGCGGCAACACCTACTATGCCTCCTGGGCCAAGGGCCGCTTCACAA |
| | | TCTCTAAGGCCTCCTCTACCACAGTGACCCTGCAGCTGACAAGCCTGAC |
| | | CGCCGCCGATACAGCCACCTACTTCTGTGCCCGGTTTGACGTGGATGGC |
| | | TCCGCCTTTAATCTGTGGGGACCAGGCACACTGGTGACCGTGAGCTCC |
| 174 | 18558 | QVQLKQSGPGLVQPSQSLSITCTVSGFSLTSYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPSSVSAAVGGTVTIKCQASQTIGSSLAWYQQKPGQPPKLLIYRASTLASGV |
| | | SSRFRGSGSGTEYTLTISDLECADAATYYCQWTDYGYIYIWAFGGGTEVVV |
| | | KGGGGSGGGGSGGGGSQEQLVESGGGLVQPEGSLILTCTASKFSFSSLYY |
| | | MCWVRQAPGKGLEWIACVYGGSSGNTYYASWAKGRFTISKASSTTVTLQL |
| | | TSLTAADTATYFCARFDVDGSGFNLWGPGTLVTVSS |
| 175 | 18558 | CAGGTGCAGCTGAAGCAGTCCGGACCAGGACTGGTGCAGCCTTCTCAG |
| | | AGCCTGTCCATCACCTGCACAGTGAGCGGCTTCTCCCTGACCTCTTACG |
| | | GCGTGCACTGGGTGAGGCAGTCTCCTGGCAAGGGACTGGAGTGGCTGG |
| | | GCGTGATCTGGAGCGGAGGCTCCACAGACTATAACGCCGCCTTTATCTC |
| | | TCGCCTGTCTATCAGCAAGGATAACTCCAAGTCTCAGGTGTTCTTTAAG |
| | | ATGAATAGCCTGCAGGCCGACGATACAGCCATCTACTATTGTGCCCGGA |
| | | ATCCCCTGACCGCCACAGTGATGGACTACTGGGGCCAGGGCACCAGCG |
| | | TGACAGTGAGCTCCGCTAGCACAAAGGGCCCCTCCGTGTTTCCTCTGGC |
| | | CCCATCCTCTAAGTCCACCTCTGGAGGAACAGCCGCCCTGGGCTGTCTG |
| | | GTGAAGGATTACTTCCCTGAGCCAGTGACCGTGTCCTGGAACTCTGGGG |
| | | CCCTGACCAGCGGAGTGCACACATTTCCCGCCGTGCTGCAGAGCTCCGG |
| | | ACTGTACTCCCTGTCTAGCGTGGTGACCGTGCCTTCCTCTAGCCTGGGCA |
| | | CCCAGACATATATCTGCAACGTGAATCACAAGCCTTCCAATACAAAGGT |
| | | CGACAAGAAGGTGGAGCCAAAGTCTTGTGATAAGACCCACACATGCCC |
| | | ACCTTGTCCGGCGCCAGAGGCCGCCGGAGGACCAAGCGTGTTCCTGTTT |
| | | CCACCCAAGCCCAAGGACACCCTGATGATCTCCCGGACCCCAGAGGTG |
| | | ACATGCGTGGTGGTGAGCGTGTCCCACGAGGACCCCGAGGTGAAGTTT |
| | | AACTGGTACGTGGATGGCGTGGAGGTGCACAATGCCAAGACAAAGCCC |
| | | CGGGAGGAGCAGTACAATTCTACCTATAGAGTGGTGAGCGTGCTGACA |
| | | GTGCTGCACCAGGATTGGCTGAACGGCAAGGAGTATAAGTGTAAGGTG |
| | | AGCAATAAGGCCCTGCCAGCCCCCATCGAGAAGACCATCTCCAAGGCC |
| | | AAGGGCCAGCCTCGCGAGCCCCAGGTGTACGTGTATCCCCCTAGCAGA |
| | | GACGAGCTGACAAAGAACCAGGTGTCCCTGACCTGCCTGGTGAAGGGC |
| | | TTCTATCCCTCTGATATCGCCGTGGAGTGGGAGAGCAATGGCCAGCCTG |
| | | AGAACAATTACAAGACCACACCACCCGTGCCGGACAGCGATGGCTCCTT |
| | | CGCCCTGGTGAGCAAGCTGACAGTGGACAAGTCCAGGTGGCAGCAGGG |
| | | CAACGTGTTTTCTTGCAGCGTGATGCACGAGGCCCTGCACAATCACTAT |
| | | ACCCAGAAGTCCCTGTCTCTGAGCCCAGGAGGAGGAGGAGGCGATATC |
| | | GTGATGACCCAGACACCAAGCTCCGTGAGCGCCGCCGTGGGAGGAACC |
| | | GTGACAATCAAGTGTCAGGCCTCCCAGACCATCGGCTCTAGCCTGGCCT |
| | | GGTATCAGCAGAAGCCTGGCCAGCCTCCAAAGCTGCTGATCTACAGAG |
| | | CCTCCACACTGGCCTCTGGCGTGTCCTCTCGGTTCAGAGGCTCCGGCTCT |
| | | GGCACCGAGTACACCCTGACAATCAGCGACCTGGAGTGCGCAGATGCA |
| | | GCAACATACTATTGTCAGTGGACCGACTACGGCTATATCTACATCTGGG |
| | | CCTTTGGCGGAGGAACCGAGGTGGTGGTGAAGGGAGGAGGAGGCAGCG |
| | | GCGGCGGAGGCTCCGGCGGCGGCGGCTCTCAGGAGCAGCTGGTGGAGT |
| | | CTGGAGGAGGACTGGTGCAGCCTGAGGGCTCCCTGACCCTGACATGCA |
| | | CCGCCTCTAAGTTCAGCTTTAGCTCCCTGTACTATATGTGCTGGGTGAGG |
| | | CAGGCCCCAGGCAAGGGACTGGAGTGGATCGCCTGCGTGTATGGCGGC |
| | | TCTAGCGGCAACACCTACTATGCCTCCTGGGCCAAGGGCCGCTTCACAA |
| | | TCTCTAAGGCCTCCTCTACCACAGTGACCCTGCAGCTGACAAGCCTGAC |
| | | CGCCGCCGATACAGCCACCTACTTCTGTGCCCGGTTTGACGTGGATGGC |
| | | TCCGGCTTTAATCTGTGGGGACCAGGCACACTGGTGACCGTGAGCTCC |
| 176 | 18559 | QVQLKESGPGLVAPSQSLSITCTVSGFSLTSYAINWVRQPPGKGLEWLGVL |
| | | WPGGGTNYNSALKSRLSISKDNSKSQVFLKMNSLQTDDTARYYCARGSGT |
| | | WYFDVWGAGTTVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPSSVSAAVGGTVTIKCQASQTIGSSLAWYQQKPGQPPKLLIYRASTLASGV |
| | | SSRFRGSGSGTEYTLTISDLECADAATYYCQWTDYGYIYIWAFGGGTEVVV |
| | | KGGGGSGGGGSGGGGSQEQLVESGGGLVQPEGSLTLTCTASKFSFSSLYY |
| | | MCWVRQAPGKGLEWIACVYGGSSGNTYYASWAKGRFTISKASSTTVTLQL |
| | | TSLTAADTATYFCARFDVDGSGFNLWGPGTLVTVSS |
| 177 | 18559 | CAGGTGCAGCTGAAGGAGAGCGGACCAGGACTGGTGGCCCCCTCTCAG |
| | | AGCCTGTCCATCACCTGCACAGTGAGCGGCTTTTCCCTGACCTCTTACGC |
| | | CATCAACTGGGTGCGGCAGCCACCTGGCAAGGGACTGGAGTGGCTGGG |
| | | CGTGCTGTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAACAGCCTGCAGACCGACGATACAGCCCGGTACTATTGTGCCAGA |
| | | GGCTCCGGCACCTGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAGCCCCAGGTGTACGTGTATCCCCCTAGCAGAGAC |
| | | GAGCTGACAAAGAACCAGGTGTCCCTGACCTGCCTGGTGAAGGGCTTCT |
| | | ATCCCTCTGATATCGCCGTGGAGTGGGAGAGCAATGGCCAGCCTGAGA |
| | | ACAATTACAAGACCACACCACCCGTGCTGGACAGCGATGGCTCCTTCGC |
| | | CCTGGTGAGCAAGCTGACAGTGGACAAGTCCAGGTGGCAGCAGGGCAA |
| | | CGTGTTTTCTTGCAGCGTGATGCACGAGGCCCTGCACAATCACTATACC |
| | | CAGAAGTCCCTGTCTCTGAGCCCAGGAGGAGGAGGAGGCGATATCGTG |
| | | ATGACCCAGACACCAAGCTCCGTGAGCGCCGCCGTGGGAGGAACCGTG |
| | | ACAATCAAGTGTCAGGCCTCCCAGACCATCGGCTCTAGCCTGGCCTGGT |
| | | ATCAGCAGAAGCCTGGCCAGCCTCCAAAGCTGCTGATCTACAGAGCCTC |
| | | CACACTGGCCTCTGGCGTGTCCTCTCGGTTCAGAGGCTCCGGCTCTGGC |
| | | ACCGAGTACACCCTGACAATCAGCGACCTGGAGTGCGCAGATGCAGCA |
| | | ACATACTATTGTCAGTGGACCGACTACGGCTATATCTACATCTGGGCCT |
| | | TTGGCGGAGGAACCGAGGTGGTGGTGAAGGGAGGAGGAGGCAGCGGC |
| | | GGCGGAGGCTCCGGCGGCGGCGGCTCTCAGGAGCAGCTGGTGGAGTCT |
| | | GGAGGAGGACTGGTGCAGCCTGAGGGCTCCCTGACCCTGACATGCACC |
| | | GCCTCTAAGTTCAGCTTTAGCTCCCTGTACTATATGTGCTGGGTGAGGC |
| | | AGGCCCCAGGCAAGGGACTGGAGTGGATCGCCTGCGTGTATGGCGGCT |
| | | CTAGCGGCAACACCTACTATGCCTCCTGGGCCAAGGGCCGCTTCACAAT |
| | | CTCTAAGGCCTCCTCTACCACAGTGACCCTGCAGCTGACAAGCCTGACC |
| | | GCCGCCGATACAGCCACCTACTTCTGTGCCCGGTTTGACGTGGATGGCT |
| | | CCGGCTTTAATCTGTGGGGACCAGGCACACTGGTGACCGTGAGCTCC |
| 178 | 18560 | QVQLQESGPGLVQPTQSLSITCTVSGFSLISYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPSSVSAAVGGTVTIKCQASQTIGSSLAWYQQKPGQPPKLLIYRASTLASGV |
| | | SSRFRGSGSGTEYTLTISDLECADAATYYCQWTDYGYIYIWAFGGGTEVVV |
| | | KGGGGSGGGGSGGGGSQEQLVESGGGLVQPEGSLTLTCTASKFSFSSLYY |
| | | MCWVRQAPGKGLEWIACVYGGSSGNTYYASWAKGRFTISKASSTTVTLQL |
| | | TSLTAADTATYFCARFDVDGSGFNLWGPGTLNTVSS |
| 179 | 18560 | CAGGTGCAGCTGCAGGAGAGCGGACCAGGACTGGTGCAGCCTACACAG |
| | | TCTCTGAGCATCACCTGCACAGTGTCTGGCTTCAGCCTGATCTCCTACGG |
| | | AGTGCACTGGGTGAGGCAGTCCCCTGGCAAGGGACTGGAGTGGCTGGG |
| | | CGTGATCTGGTCTGGCGGCAGCACCGACTATAACGCCGCCTTTATCTCC |
| | | CGCCTGTCCATCTCTAAGGATAACAGCAAGTCCCAGGTGTTCTTTAAGA |
| | | TGAACAGCCTGCAGGCCGACGATACAGCCATCTACTATTGTGCCCGGAA |
| | | TCCCCTGACCGCCACAGTGATGGACTACTGGGGCCAGGGCACCTCTGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAGCCCCAGGTGTACGTGTATCCCCCTAGCAGAGAC |
| | | GAGCTGACAAAGAACCAGGTGTCCCTGACCTGCCTGGTGAAGGGCTTCT |
| | | ATCCCTCTGATATCGCCGTGGAGTGGGAGAGCAATGGCCAGCCTGAGA |
| | | ACAATTACAAGACCACACCACCCGTGCTGGACAGCGATGGCTCCTTCGC |
| | | CCTGGTGAGCAAGCTGACAGTGGACAAGTCCAGGTGGCAGCAGGGCAA |
| | | CGTGTTTTCTTGCAGCGTGATGCACGAGGCCCTGCACAATCACTATACC |
| | | CAGAAGTCCCTGTCTCTGAGCCCAGGAGGAGGAGGAGGCGATATCGTG |
| | | ATGACCCAGACACCAAGCTCCGTGAGCGCCGCCGTGGGAGGAACCGTG |
| | | ACAATCAAGTGTCAGGCCTCCCAGACCATCGGCTCTAGCCTGGCCTGGT |
| | | ATCAGCAGAAGCCTGGCCAGCCTCCAAAGCTGCTGATCTACAGAGCCTC |
| | | CACACTGGCCTCTGGCGTGTCCTCTCGGTTCAGAGGCTCCGGCTCTGGC |
| | | ACCGAGTACACCCTGACAATCAGCGACCTGGAGTGCGCAGATGCAGCA |
| | | ACATACTATTGTCAGTGGACCGACTACGGCTATATCTACATCTGGGCCT |
| | | TTGGCGGAGGAACCGAGGTGGTGGTGAAGGGAGGAGGAGGCAGCGGC |
| | | GGCGGAGGCTCCGGCGGCGGCGGCTCTCAGGAGCAGCTGGTGGAGTCT |
| | | GGAGGAGGACTGGTGCAGCCTGAGGGCTCCCTGACCCTGACATGCACC |
| | | GCCTCTAAGTTCAGCTCTAGCTCCCTGTACTATATGTGCTGGGTGAGGC |
| | | AGGCCCCAGGCAAGGGACTGGAGTGGATCGCCTGCGTGTATGGCGGCT |
| | | CTAGCGGCAACACCTACTATGCCTCCTGGGCCAAGGGCCGCTTCACAAT |
| | | CTCTAAGGCCTCCTCTACCACAGTGACCCTGCAGCTGACAAGCCTGACC |
| | | GCCGCCGATACAGCCACCTACTTCTGTGCCCGGTTTGACGTGGATGGCT |
| | | CCGGCTTTAATCTGTGGGGACCAGGCACACTGGTGACCGTGAGCTCC |
| 180 | 18561 | QVQLKESGPGLVAPSQSLSITCTVSGFSLTSYAISWVRQPPGKGLEWLGVIW |
| | | PGGGTNYNSALKSRLSISKDNSKSQVFLKMNSLQTDDTARYYCARGTGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPSSVSAAVGGTVTIKCQASQTIGSSLAWYQQKPGQPPKLLIYRASTLASGV |
| | | SSRFRGSGSGTEYTLTISDLECADAATYYCQWTDYGYIYIWAFGGGTEVVV |
| | | KGGGGSGGGGSGGGGSQEQLVESGGGLVQPEGSLTLTCTASKFSFSSLYY |
| | | MCWVRQAPGKGLEWIACVYGGSSGNTYYASWAKGRFTISKASSTTVTLQL |
| | | TSLTAADTATYFCARFDVDGSGFNLWGPGTLNTVSS |
| 181 | 18561 | CAGGTGCAGCTGAAGGAGTCTGGACCAGGACTGGTGGCCCCCTCTCAG |
| | | AGCCTGTCCATCACCTGCACAGTGAGCGGCTTTTCCCTGACCTCTTACGC |
| | | AATCTCCTGGGTGCGGCAGCCACCTGGCAAGGGACTGGAGTGGCTGGG |
| | | CGTGATCTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAATAGCCTGCAGACCGACGATACAGCCCGGTACTATTGTGCCAGA |
| | | GGCACCGGCACATGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAGCCCCAGGTGTACGTGTATCCCCCTAGCAGAGAC |
| | | GAGCTGACAAAGAACCAGGTGTCCCTGACCTGCCTGGTGAAGGGCTTCT |
| | | ATCCCTCTGATATCGCCGTGGAGTGGGAGAGCAATGGCCAGCCTGAGA |
| | | ACAATTACAAGACCACACCACCCGTGCTGGACAGCGATGGCTCCTTCGC |
| | | CCTGGTGAGCAAGCTGACAGTGGACAAGTCCAGGTGGCAGCAGGGCAA |
| | | CGTGTTTTCTTGCAGCGTGATGCACGAGGCCCTGCACAATCACTATACC |
| | | CAGAAGTCCCTGTCTCTGAGCCCAGGAGGAGGAGGAGGCGATATCGTG |
| | | ATGACCCAGACACCAAGCTCCGTGAGCGCCGCCGTGGGAGGAACCGTG |
| | | ACAATCAAGTGTCAGGCCTCCCAGACCATCGGCTCTAGCCTGGCCTGGT |
| | | ATCAGCAGAAGCCTGGCCAGCCTCCAAAGCTGCTGATCTACAGAGCCTC |
| | | CACACTGGCCTCTGGCGTGTCCTCTCGGTTCAGAGGCTCCGGCTCTGGC |
| | | ACCGAGTACACCCTGACAATCAGCGACCTGGAGTGCGCAGATGCAGCA |
| | | ACATACTATTGTCAGTGGACCGACTACGGCTATATCTACATCTGGGCCT |
| | | TTGGCGGAGGAACCGAGGTGGTGGTGAAGGGAGGAGGAGGCAGCGGC |
| | | GGCGGAGGCTCCGGCGGCGGCGGCTCTCAGGAGCAGCTGGTGGAGTCT |
| | | GGAGGAGGACTGGTGCAGCCTGAGGGCTCCCTGACCCTGACATGCACC |
| | | GCCTCTAAGTTCAGCTCTAGCTCCCTGTACTATATGTGCTGGGTGAGGC |
| | | AGGCCCCAGGCAAGGGACTGGAGTGGATCGCCTGCGTGTATGGCGGCT |
| | | CTAGCGGCAACACCTACTATGCCTCCTGGGCCAAGGGCCGCTTCACAAT |
| | | CTCTAAGGCCTCCTCTACCACAGTGACCCTGCAGCTGACAAGCCTGACC |
| | | GCCGCCGATACAGCCACCTACTTCTGTGCCCGGTTTGACGTGGATGGCT |
| | | CCGGCTTTAATCTGTGGGGACCAGGCACACTGGTGACCGTGAGCTCC |
| 182 | 14471 | DIQMTQSTSSLSASVGDRVTISCSASQGISNYLNWYQQKPDGTVKLLIYYTS |
| | | SLHSGVPSRFSGSGSGTDYSLTISNLEPEDIATYYCQQYSKFPWTFGGGTKL |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 183 | 14471 | GACATCCAGATGACCCAGTCCACAAGCTCCCTGTCCGCCTCTGTGGGCG |
| | | ATAGAGTGACAATCAGCTGCTCCGCCTCTCAGGGCATCTCTAACTACCT |
| | | GAATTGGTATCAGCAGAAGCCTGACGGCACCGTGAAGCTGCTGATCTAC |
| | | TATACATCTAGCCTGCACAGCGGAGTGCCATCCAGGTTTAGCGGCTCCG |
| | | GCTCTGGCACCGACTACTCTCTGACAATCAGCAACCTGGAGCCCGAGGA |
| | | TATCGCCACCTACTATTGTCAGCAGTATAGCAAGTTCCCTTGGACCTTTG |
| | | GCGGCGGCACAAAGCTGGAGATCAAGCGCACAGTGGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTCAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 184 | 4553 | EVQLVESGGGLVQPGGSLRLSCAASGFNIKDTYIHWVRQAPGKGLEWVAR |
| | | IYPTNGYTRYADSVKGRFTISADTSKNTAYLQMNSLRAEDTAVYYCSRWG |
| | | GDGFYAMDYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKKVEPKSCDKTHTCPPCPAPELLGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYV |
| | | YPPSRDELTKNQVSLTCLVKGFYPSDIAVEWESNGQPENNYKTTPPVLDSD |
| | | GSFALVSKLTVDKSRWQQGNVFSCSVMHEALHNHYTQKSLSLSPGK |
| 185 | 4553 | GAAGTCCAGCTGGTCGAAAGCGGAGGAGGACTGGTGCAGCCAGGAGGG |
| | | TCTCTGCGACTGAGTTGCGCCGCTTCAGGCTTCAACATCAAGGACACCT |
| | | ACATTCACTGGGTGCGCCAGGCTCCTGGAAAAGGCCTGGAGTGGGTGG |
| | | CACGAATCTATCCAACTAATGGATACACCCGGTATGCAGACAGCGTGA |
| | | AGGGCCGGTTCACCATTAGCGCAGATACATCCAAAAACACTGCCTACCT |
| | | GCAGATGAACAGCCTGCGAGCCGAAGATACTGCTGTGTACTATTGCAGT |
| | | CGGTGGGGAGGCGACGGCTTCTACGCTATGGATTATTGGGGGCAGGGA |
| | | ACCCTGGTCACAGTGAGCTCCGCATCTACAAAGGGGCCTAGTGTGTTTC |
| | | CACTGGCCCCCTCTAGTAAATCCACCTCTGGGGGAACAGCAGCCCTGGG |
| | | ATGTCTGGTGAAGGACTATTTCCCAGAGCCCGTCACTGTGAGTTGGAAC |
| | | TCAGGCGCCCTGACATCCGGGGTCCATACTTTTCCTGCTGTGCTGCAGTC |
| | | AAGCGGCCTGTACTCTCTGTCCTCTGTGGTCACCGTGCCAAGTTCAAGC |
| | | CTGGGGACTCAGACCTATATCTGCAACGTGAATCACAAGCCAAGCAAT |
| | | ACAAAAGTCGACAAGAAAGTGGAACCCAAGAGCTGTGATAAAACACAT |
| | | ACTTGCCCCCCTTGTCCTGCACCAGAGCTGCTGGGAGGACCATCCGTGT |
| | | TCCTGTTTCCACCCAAGCCTAAAGACACCCTGATGATTTCCAGGACTCC |
| | | AGAAGTCACCTGCGTGGTCGTGGACGTGTCTCACGAGGACCCCGAAGTC |
| | | AAGTTCAACTGGTACGTGGATGGCGTCGAGGTGCATAATGCCAAGACA |
| | | AAACCCAGGGAGGAACAGTACAACTCAACTTATCGCGTCGTGAGCGTC |
| | | CTGACCGTGCTGCACCAGGACTGGCTGAACGGCAAGGAGTATAAGTGC |
| | | AAAGTGAGCAATAAGGCTCTGCCCGCACCTATCGAGAAAACCATTAGC |
| | | AAGGCCAAAGGGCAGCCTAGAGAACCACAGGTCTACGTGTATCCTCCA |
| | | AGCAGGGACGAGCTGACCAAGAACCAGGTCTCCCTGACATGTCTGGTG |
| | | AAAGGGTTTTACCCCAGTGATATCGCTGTGGAGTGGGAATCAAATGGAC |
| | | AGCCTGAAAACAATTATAAGACCACACCCCCTGTGCTGGACAGCGATG |
| | | GCAGCTTCGCTCTGGTCTCCAAGCTGACTGTGGATAAATCTCGGTGGCA |
| | | GCAGGGCAACGTCTTTAGTTGTTCAGTGATGCATGAGGCACTGCACAAT |
| | | CATTACACCCAGAAGAGCCTGTCCCTGTCTCCCGGCAAA |
| 186 | 10443 | DIQMTQSPSSLSASVGDRVTITCRASQDVNTAVAWYQQKPGKAPKLLIYSA |
| | | SFYSGVPSRFSGSRSGTDFTLTISSLQPEDFATYYCQQHYTTPPTFGQGTKV |
| | | EIKGGSGGGSGGGSGGGSGGGSGEVQLVESGGGLVQPGGSLRLSCAASGF |
| | | NIKDTYIHWVRQAPGKGLEWVARIYPTNGYTRYADSVKGRFTISADTSKNT |
| | | AYLQMNSLRAEDTAVYYCSRWGGDGFYAMDYWGQGTLVTVSSGGEVQL |
| | | VQSGAEVKKPGESLRISCKGSGYSFSTYWISWVRQMPGKGLEWMGKIYPG |
| | | DSYTNYSPSFQGQVTISADKSISTAYLQWSSLKASDTAMYYCARGYGIFDY |
| | | WGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISRTPEVTC |
| | | VVVSVSHEDPEVKFNWYVDGVEVHNAKTKPREEQYNSTYRVVSVLTVLH |
| | | QDWLNGKEYKCKVSNKALPAPIEKTISKAKGQPREPQVYVLPPSRDELTKN |
| | | QVSLLCLVKGFYPSDIAVEWESNGQPENNYLTWPPVLDSDGSFFLYSKLTV |
| | | DKSRWQQGNVFSCSVMHEALHNHYTQKSLSLSPG |
| 187 | 10443 | GACATCCAGATGACACAGTCCCCAAGCTCCCTGTCCGCCTCTGTGGGCG |
| | | ACAGGGTGACCATCACATGCAGGGCCAGCCAGGATGTGAACACCGCCG |
| | | TGGCCTGGTACCAGCAGAAGCCAGGCAAGGCCCCCAAGCTGCTGATCT |
| | | ACAGCGCCTCCTTCCTGTATTCTGGCGTGCCAAGCCGGTTTTCTGGCAGC |
| | | AGATCCGGCACCGACTTCACCCTGACAATCTCTAGCCTGCAGCCCGAGG |
| | | ATTTTGCCACATACTATTGCCAGCAGCACTACACCACACCCCCTACCTTC |
| | | GGCCAGGGCACAAAGGTGGAGATCAAGGGCGGCTCCGGCGGCGGCTCT |
| | | GGAGGAGGCAGCGGAGGAGGCTCCGGCGGAGGCTCTGGCGAGGTGCA |
| | | GCTGGTGGAGAGCGGAGGAGGCCTGGTGCAGCCAGGAGGCAGCCTGAG |
| | | ACTGTCCTGTGCCGCCTCTGGCTTTAACATCAAGGACACCTACATCCAC |
| | | TGGGTGCGGCAGGCCCCCGGCAAGGGCCTGGAGTGGGTGGCCAGAATC |
| | | TATCCTACCAATGGCTACACACGGTATGCCGACTCCGTGAAGGGCCGGT |
| | | TCACCATCAGCGCCGATACCTCCAAGAACACAGCCTACCTGCAGATGAA |
| | | CAGCCTGCGGGCCGAGGATACAGCCGTGTACTATTGCAGCCGCTGGGG |
| | | AGGCGACGGCTTCTACGCCATGGATTATTGGGGCCAGGGCACCCTGGTG |
| | | ACAGTGTCCTCTGGCGGAGAAGTGCAGCTGGTGCAGTCTGGAGCCGAG |
| | | GTGAAGAAGCCCGGCGAGTCTCTGAGGATCAGCTGTAAGGGCTCCGGC |
| | | TACTCTTTTAGCACCTATTGGATCTCCTGGGTGCGCCAGATGCCTGGCA |
| | | AGGGCCTGGAATGGATGGGCAAGATCTACCCTGGCGACTCCTACACAA |
| | | ATTATTCCCCATCTTTCCAGGGCCAGGTGACCATCTCTGCCGATAAGAG |
| | | CATCTCCACAGCCTATCTGCAGTGGAGCTCCCTGAAGGCCAGCGACACC |
| | | GCCATGTACTATTGTGCCAGAGGCTACGGCATCTTCGACTACTGGGGAC |
| | | AGGGCACCCTGGTCACAGTGTCTAGCGCTAGCACTAAGGGGCCTTCCGT |
| | | GTTTCCACTGGCTCCCTCTAGTAAATCCACCTCTGGAGGCACAGCTGCA |
| | | CTGGGATGTCTGGTGAAGGATTACTTCCCTGAACCAGTCACAGTGAGTT |
| | | GGAACTCAGGGGCTCTGACAAGTGGAGTCCATACTTTTCCCGCAGTGCT |
| | | GCAGTCAAGCGGACTGTACTCCCTGTCCTCTGTGGTCACCGTGCCTAGT |
| | | TCAAGCCTGGGCACCCAGACATATATCTGCAACGTGAATCACAAGCCAT |
| | | CAAATACAAAAGTCGACAAGAAAGTGGAGCCCAAGAGCTGTGATAAAA |
| | | CTCATACCTGCCCACCTTGTCCGGCGCCAGAGGCAGCAGGAGGACCAA |
| | | GCGTGTTCCTGTTTCCACCCAAGCCCAAAGACACCCTGATGATTAGCCG |
| | | AACCCCTGAAGTCACATGCGTGGTCGTGTCCGTGTCTCACGAGGACCCA |
| | | GAAGTCAAGTTCAACTGGTACGTGGATGGCGTCGAGGTGCATAATGCC |
| | | AAGACAAAACCCCGGGAGGAACAGTACAACAGCACCTATAGAGTCGTG |
| | | TCCGTCCTGACAGTGCTGCACCAGGATTGGCTGAACGGCAAGGAATATA |
| | | AGTGCAAAGTGTCCAATAAGGCCCTGCCCGCTCCTATCGAGAAAACCAT |
| | | TTCTAAGGCAAAAGGCCAGCCTCGCGAACCACAGGTCTACGTGCTGCCT |
| | | CCATCCCGGGACGAGCTGACAAAGAACCAGGTCTCTCTGCTGTGCCTGG |
| | | TGAAAGGCTTCTATCCATCAGATATTGCTGTGGAGTGGGAAAGCAATGG |
| | | GCAGCCCGAGAACAATTACCTGACTTGGCCCCCTGTGCTGGACTCTGAT |
| | | GGGAGTTTCTTTCTGTATTCTAAGCTGACCGTGGATAAAAGTAGGTGGC |
| | | AGCAGGGAAATGTCTTTAGTTGTTCAGTGATGCATGAAGCCCTGCATAA |
| | | CCACTACACCCAGAAAAGCCTGTCCCTGTCCCCCGGA |
| 188 | 10445 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNYNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGDIQMTQSPS |
| | | SLSASVGDRVTITCRASQDVNTAVAWYQQKPGKAPKLLIYSASFLYSGVPS |
| | | RFSGSRSGTDFTLTISSLQPEDFATYYCQQHYTTPPTFGQGTKVEIKGGSGG |
| | | GSGGGSGGGSGGGSGEVQLVESGGGLVQPGGSLRLSCAASGFNIKDTYIH |
| | | WVRQAPGKGLEWVARIYPTNGYTRYADSVKGRFTISADTSKNTAYLQMN |
| | | SLRAEDTAVYYCSRWGGDGFYAMDWGQGTLVTVSS |
| 189 | 10445 | GAGGTGCAGCTGGTGCAGTCCGGAGCAGAGGTGAAGAAGCCTGGCGAG |
| | | AGCCTGAGGATCTCCTGCAAGGGCTCTGGCTACTCCTTTTCTACCTATTG |
| | | GATCAGCTGGGTGCGCCAGATGCCAGGCAAGGGCCTGGAGTGGATGGG |
| | | CAAGATCTACCCTGGCGACTCCTACACAAACTATAGCCCATCCTTCCAG |
| | | GGCCAGGTGACCATCTCTGCCGATAAGTCTATCAGCACAGCCTATCTGC |
| | | AGTGGAGCTCCCTGAAGGCCTCCGACACCGCCATGTACTATTGCGCCAG |
| | | AGGCTACGGCATCTTTGATTATTGGGGCCAGGGCACCCTGGTGACAGTG |
| | | TCTAGCGCTAGCACAAAGGGCCCTTCCGTGTTCCCTCTGGCCCCATCCTC |
| | | TAAGTCCACCTCTGGAGGAACAGCCGCCCTGGGCTGTCTGGTGAAGGAC |
| | | TACTTTCCTGAGCCAGTGACCGTGTCTTGGAACAGCGGGGCCCTGACCA |
| | | GCGGAGTGCACACATTCCCCGCCGTGCTGCAGAGCTCCGGCCTGTACTC |
| | | CCTGTCTAGCGTGGTGACCGTGCCTTCCTCTAGCCTGGGCACCCAGACA |
| | | TATATCTGCAACGTGAATCACAAGCCATCCAATACAAAGGTCGACAAG |
| | | AAGGTGGAGCCCAAGTCTTGTGATAAGACCCACACATGCCCACCTTGTC |
| | | CGGCGCCAGAGGCCGCCGGAGGACCAAGCGTGTTCCTGTTTCCACCCAA |
| | | GCCTAAGGACACCCTGATGATCTCCAGGACCCCTGAGGTGACATGCGTG |
| | | GTGGTGAGCGTGTCCCACGAGGACCCAGAGGTGAAGTTTAACTGGTAC |
| | | GTGGATGGCGTGGAGGTGCACAATGCCAAGACCAAGCCCAGGGAGGAG |
| | | CAGTACAACTCTACCTATCGCGTGGTGAGCGTGCTGACAGTGCTGCACC |
| | | AGGATTGGCTGAACGGCAAGGAGTATAAGTGCAAGGTGTCTAATAAGG |
| | | CCCTGCCAGCCCCCATCGAGAAGACCATCAGCAAGGCCAAGGGCCAGC |
| | | CTCGCGAACCTCAGGTGTACGTGCTGCCTCCATCTAGAGACGAGCTGAC |
| | | AAAGAACCAGGTGAGCCTGCTGTGCCTGGTGAAGGGCTTTTATCCCAGC |
| | | GATATCGCCGTGGAGTGGGAGTCCAATGGCCAGCCTGAGAACAATTAC |
| | | CTGACCTGGCCCCCTGTGCTGGACTCCGATGGCTCTTTCTTTCTGTATTC |
| | | CAAGCTGACAGTGGACAAGTCTCGCTGGCAGCAGGGCAACGTGTTCTCT |
| | | TGCAGCGTGATGCACGAGGCCCTGCACAATCACTACACCCAGAAGTCCC |
| | | TGAGCTTAAGCCCAGGAGGAGGAGGAGGCGATATCCAGATGACACAGA |
| | | GCCCATCCTCTCTGTCCGCCTCTGTGGGCGACAGGGTGACCATCACATG |
| | | TCGCGCCTCCCAGGATGTGAATACCGCCGTGGCCTGGTATCAGCAGAAG |
| | | CCAGGCAAGGCCCCCAAGCTGCTGATCTACAGCGCCTCCTTCCTGTATA |
| | | GCGGCGTGCCATCCCGGTTTTCTGGCAGCAGATCCGGCACCGACTTCAC |
| | | CCTGACAATCAGCTCCCTGCAGCCCGAGGATTTTGCCACATACTATTGC |
| | | CAGCAGCACTACACCACACCACCCACCTTCGGCCAGGGCACAAAGGTG |
| | | GAGATCAAGGGAGGCTCTGGAGGAGGCAGCGGAGGAGGCTCCGGAGG |
| | | AGGCTCTGGCGGCGGCAGCGGCGAGGTGCAGCTGGTGGAGAGCGGCGG |
| | | CGGCCTGGTGCAGCCCGGCGGCTCCCTGCGGCTGTCTTGTGCCGCCAGC |
| | | GGCTTCAACATCAAGGACACCTACATCCACTGGGTGCGGCAGGCACCTG |
| | | GCAAGGGCCTGGAATGGGTGGCCAGAATCTATCCAACCAATGGCTACA |
| | | CACGGTATGCCGACAGCGTGAAGGGCCGGTTCACCATCTCCGCCGATAC |
| | | CTCTAAGAACACAGCCTACCTGCAGATGAATAGCCTGAGGGCCGAGGA |
| | | TACAGCCGTGTACTATTGCTCCAGATGGGGAGGCGACGGCTTCTACGCC |
| | | ATGGACTACTGGGGACAGGGCACCCTGGTCACAGTGTCTAGC |
| 190 | 21112 | EVQLVESGGGLIQPGGSLRLSCAASGFSLTTYAISWVRQAPGKGLEWYSIIW |
| | | PGGGTNYADSVKGRFTISRDNSKNTLYLQMNSLRAEDTAVYYCARGAGT |
| | | WYFDVWGQGTLVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 191 | 21112 | GAGGTGCAGCTGGTGGAGTCCGGAGGAGGACTGATCCAGCCAGGAGGC |
| | | TCCCTGCGGCTGTCTTGCGCCGCCTCTGGCTTTAGCCTGACCACATACGC |
| | | CATCTCTTGGGTGAGACAGGCCCCCGGCAAGGGACTGGAGTGGGTGTCT |
| | | ATCATCTGGCCTGGCGGCGGCACAAACTACGCCGACAGCGTGAAGGGC |
| | | CGGTTCACCATCAGCAGAGATAACTCCAAGAATACACTGTATCTGCAGA |
| | | TGAATAGCCTGAGGGCCGAGGACACCGCCGTGTACTATTGTGCCCGGG |
| | | GGGCAGGGACATGGTATTTTGACGTGTGGGGCCAGGGCACCCTGGTGA |
| | | CAGTGAGCTCCGCTAGCACAAAGGGCCCCTCCGTGTTTCCTCTGGCCCC |
| | | ATCCTCTAAGTCCACCTCTGGAGGAACAGCCGCCCTGGGCTGTCTGGTG |
| | | AAGGATTACTTCCCTGAGCCAGTGACCGTGTCCTGGAACTCTGGGGCCC |
| | | TGACCAGCGGAGTGCACACATTTCCCGCCGTGCTGCAGAGCTCCGGACT |
| | | GTACTCCCTGTCTAGCGTGGTGACCGTGCCTTCCTCTAGCCTGGGCACCC |
| | | AGACATATATCTGCAACGTGAATCACAAGCCTTCCAATACAAAGGTCGA |
| | | CAAGAAGGTGGAGCCAAAGTCTTGTGATAAGACCCACACATGCCCACC |
| | | TTGTCCGGCGCCAGAGGCCGCCGGAGGACCAAGCGTGTTCCTGTTTCCA |
| | | CCCAAGCCCAAGGACACCCTGATGATCTCCCGGACCCCAGAGGTGACA |
| | | TGCGTGGTGGTGAGCGTGTCCCACGAGGACCCCGAGGTGAAGTTTAACT |
| | | GGTACGTGGATGGCGTGGAGGTGCACAATGCCAAGACAAAGCCCCGGG |
| | | AGGAGCAGTACAATTCTACCTATAGAGTGGTGAGCGTGCTGACAGTGCT |
| | | GCACCAGGATTGGCTGAACGGCAAGGAGTATAAGTGTAAGGTGAGCAA |
| | | TAAGGCCCTGCCAGCCCCCATCGAGAAGACCATCTCCAAGGCCAAGGG |
| | | CCAGCCTCGCGAACCACAGGTGTACACTCTGCCTCCATCTCGGGACGAG |
| | | CTGACTAAGAACCAGGTCAGTCTGACCTGTCTGGTGAAAGGATTCTATC |
| | | CCAGCGATATCGCTGTGGAGTGGGAATCCAATGGCCAGCCTGAGAACA |
| | | ATTACAAGACCACACCCCCTGTGCTGGACTCTGATGGCAGTTTCTTTCTG |
| | | TATAGTAAGCTGACCGTCGATAAATCACGATGGCAGCAGGGGAACGTG |
| | | TTCAGCTGTTCAGTGATGCACGAAGCCCTGCACAACCATTACACCCAGA |
| | | AGAGCCTGAGCCTGTCTCCCGGC |
| 192 | 21113 | EVQLVESGGGLIQPGGSLRLSCAVSGFSLTTYAISWVRQAPGKGLEWLGII |
| | | WPGGGTNYADSVKGRFTISRDNSKNTLYLQMNSLRAEDTAVYYCARGAG |
| | | TWYFDVWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPEAAGGPSVFLFPPKPKDTLMISR |
| | | TPEVTCVVVSVSHEDPEVKFNWYVDGVEVHNAKTKPREEQYNSTYRVVS |
| | | VLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFL, |
| | | YSKLTVDKSRWQQGNVFSCSVMHEALHNHYTQKSLSLSPG |
| 193 | 21113 | GAGGTGCAGCTGGTGGAGTCCGGAGGAGGACTGATCCAGCCAGGAGGC |
| | | TCCCTGCGGCTGTCTTGCGCCGTGTCTGGCTTTAGCCTGACCACATACGC |
| | | CATCTCTTGGGTGAGACAGGCCCCCGGCAAGGGACTGGAGTGGCTGGG |
| | | AATCATCTGGCCTGGAGGAGGCACAAACTACGCCGACTCTGTGAAGGG |
| | | CCGGTTCACCATCAGCAGAGATAACTCCAAGAATACACTGTATCTGCAG |
| | | ATGAATAGCCTGAGGGCCGAGGACACCGCCGTGTACTATTGTGCCCGG |
| | | GGGGCAGGGACATGGTATTTTGACGTGTGGGGCCAGGGCACCCTGGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTGTACACTCTGCCTCCATCTCGGGAC |
| | | GAGCTGACTAAGAACCAGGTCAGTCTGACCTGTCTGGTGAAAGGATTCT |
| | | ATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 194 | 21114 | EVQLVESGGGLIQPGGSLRLSCAVSGFSLTTYAISWVRQAPGKGLEWLGII |
| | | WPGGGTNYADSVKGRFTISKDNSKNTLYLQMNSLRAEDTAVYYCARGAG |
| | | TWYFDVWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPEAAGGPSVFLFPPKPKDTLMISR |
| | | TPEVTCVVVSVSHEDPEVKFNWYVDGVEVHNAKTKPREEQYNSTYRVVS |
| | | VLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFL |
| | | YSKLTVDKSRWQQGNVFSCSVMHEALHNHYTQKSLSLSPG |
| 195 | 21114 | GAGGTGCAGCTGGTGGAGTCCGGCGGCGGCCTGATCCAGCCAGGAGGC |
| | | TCCCTGAGGCTGTCTTGCGCCGTGTCTGGCTTTAGCCTGACCACATACGC |
| | | AATCTCTTGGGTGCGCCAGGCCCCCGGCAAGGGACTGGAGTGGCTGGG |
| | | AATCATCTGGCCTGGAGGAGGCACAAACTACGCCGACTCTGTGAAGGG |
| | | CCGGTTCACCATCAGCAAGGATAACTCCAAGAATACACTGTATCTGCAG |
| | | ATGAATAGCCTGCGGGCAGAGGACACCGCCGTGTACTATTGTGCCCGG |
| | | GGGGCAGGGACATGGTATTTTGACGTGTGGGGCCAGGGCACCCTGGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAACCACAGGTGTACACTCTGCCTCCATCTCGGGAC |
| | | GAGCTGACTAAGAACCAGGTCAGTCTGACCTGTCTGGTGAAAGGATTCT |
| | | ATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 196 | 4560 | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLN |
| | | GKEYKCKVSNKALPAPIEKTISKAKGQPREPQVYVLPPSRDELTKNQVSLL |
| | | CLVKGFYPSDIAVEWESNGQPENNYLTWPPVLDSDGSFFLYSKLTVDKSR |
| | | WQQGNVFSCSVMHEALHNHYTQKSLSLSPGK |
| 197 | 4560 | GAACCTAAAAGCAGCGACAAGACCCACACATGCCCCCCTTGTCCAGCTC |
| | | CAGAACTGCTGGGAGGACCAAGCGTGTTCCTGTTTCCACCCAAGCCCAA |
| | | AGATACACTGATGATCAGCCGAACTCCCGAGGTCACCTGCGTGGTCGTG |
| | | GACGTGTCCCACGAGGACCCCGAAGTCAAGTTCAACTGGTACGTGGAC |
| | | GGCGTCGAAGTGCATAATGCAAAGACTAAACCACGGGAGGAACAGTAC |
| | | AACTCTACATATAGAGTCGTGAGTGTCCTGACTGTGCTGCATCAGGATT |
| | | GGCTGAACGGCAAAGAGTATAAGTGCAAAGTGTCTAATAAGGCCCTGC |
| | | CTGCTCCAATCGAGAAAACTATTAGTAAGGCAAAAGGGCAGCCCAGGG |
| | | AACCTCAGGTCTACGTGCTGCCTCCAAGTCGCGACGAGCTGACCAAGAA |
| | | CCAGGTCTCACTGCTGTGTCTGGTGAAAGGATTCTATCCTTCCGATATTG |
| | | CCGTGGAGTGGGAATCTAATGGCCAGCCAGAGAACAATTACCTGACCT |
| | | GGCCCCCTGTGCTGGACAGCGATGGGTCCTTCTTTCTGTATTCAAAGCT |
| | | GACAGTGGACAAAAGCAGATGGCAGCAGGGAAACGTCTTTAGCTGTTC |
| | | CGTGATGCACGAAGCCCTGCACAATCATTACACCCAGAAGTCTCTGAGT |
| | | CTGTCACCTGGCAAA |
| 198 | 21115 | EVQLVESGGGLIQPGGSLRISCAVSGFSLTTYAISWVRQAPGKGLEWLGIIW |
| | | PGGGTNYADSLKGRLTISKDNSKNTVYLQMNSLRTEDTAVYYCARGAGT |
| | | WYFDVWGQGTLVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 199 | 21115 | GAGGTGCAGCTGGTGGAGTCCGGCGGCGGCCTGATCCAGCCAGGAGGC |
| | | TCCCTGAGGATCTCTTGCGCCGTGTCTGGCTTTAGCCTGACCACATACGC |
| | | AATCTCTTGGGTGCGCCAGGCCCCCGGCAAGGGACTGGAGTGGCTGGG |
| | | AATCATCTGGCCTGGAGGAGGAACCAACTACGCAGACTCTCTGAAGGG |
| | | CAGACTGACCATCAGCAAGGATAACTCCAAGAATACAGTGTATCTGCA |
| | | GATGAATAGCCTGCGGACCGAGGACACAGCCGTGTACTATTGTGCCCG |
| | | GGGGGCAGGGACATGGTATTTCGACGTGTGGGGACAGGGCACCCTGGT |
| | | GACAGTGAGCTCCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAACCACAGGTGTACACTCTGCCTCCATCTCGGGA |
| | | CGAGCTGACTAAGAACCAGGTCAGTCTGACCTGTCTGGTGAAAGGATTC |
| | | TATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 200 | 18556 | QVQLKESGPGLVAPSQSLSITCTVSGFSLTSYAISWVRQPPGKGLEWLGVIW |
| | | PGGGTNYNSALKSRLSISKDNSKSQVFLKMNSLQTDDTARYYCARGTGTW |
| | | YFDVWGAGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGDIVMTQ |
| | | TPASVEAAVGGTVTIKCQASQSIYSSLAWYQQKPGQSPKLLIYDASHLASG |
| | | VPSRFSGSRYGTEFTLTISGVQCDDAATYYCQGGWYSSAATYVPNTFGGGT |
| | | EVVVKGGGGSGGGGSGGGGSQEQLVESGGGLVQPEGSLTLTCKASGFTIS |
| | | NNYYMCWVRQAPGKGLEWIACIYGGISGRTYYASWAKGRFTISKTSSTTV |
| | | TLQMTSLTAADTATYFCVRGYVGTSNLWGPGTLVTVSS |
| 201 | 18556 | CAGGTGCAGCTGAAGGAGTCTGGACCAGGACTGGTGGCCCCCTCTCAG |
| | | AGCCTGTCCATCACCTGCACAGTGAGCGGCTTTTCCCTGACCTCTTACGC |
| | | AATCTCCTGGGTGCGGCAGCCACCTGGCAAGGGACTGGAGTGGCTGGG |
| | | CGTGATCTGGCCAGGAGGAGGCACAAACTATAATAGCGCCCTGAAGTC |
| | | CAGGCTGTCTATCAGCAAGGACAACTCCAAGTCTCAGGTGTTCCTGAAG |
| | | ATGAATAGCCTGCAGACCGACGATACAGCCCGGTACTATTGTGCCAGA |
| | | GGCACCGGCACATGGTACTTTGACGTGTGGGGGGCAGGGACCACAGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAGCCACAGGTGTACGTGTATCCCCCTAGCAGGGA |
| | | CGAGCTGACAAAGAACCAGGTGTCCCTGACCTGCCTGGTGAAGGGCTTC |
| | | TACCCCTCCGATATCGCCGTGGAGTGGGAGTCTAATGGCCAGCCTGAGA |
| | | ACAATTATAAGACCACACCACCCGTGCTGGACTCTGATGGCAGCTTCGC |
| | | CCTGGTGAGCAAGCTGACAGTGGACAAGTCCAGATGGCAGCAGGGCAA |
| | | CGTGTTTTCTTGCAGCGTGATGCACGAGGCCCTGCACAATCACTACACC |
| | | CAGAAGTCCCTGTCTCTGAGCCCAGGAGGAGGAGGAGGCGATATCGTG |
| | | ATGACCCAGACACCCGCCTCCGTGGAGGCCGCCGTGGGAGGAACCGTG |
| | | ACAATCAAGTGTCAGGCCTCCCAGTCTATCTACAGCTCCCTGGCCTGGT |
| | | ATCAGCAGAAGCCTGGCCAGAGCCCAAAGCTGCTGATCTACGACGCCT |
| | | CCCACCTGGCCTCTGGAGTGCCAAGCCGGTTCAGCGGCTCCAGATATGG |
| | | CACAGAGTTTACCCTGACAATCTCCGGAGTGCAGTGCGACGATGCAGCA |
| | | ACCTACTATTGTCAGGGAGGATGGTACTCTAGCGCCGCCACCTATGTGC |
| | | CTAACACATTCGGCGGCGGCACCGAGGTGGTGGTGAAGGGAGGAGGAG |
| | | GCTCCGGCGGAGGAGGCTCTGGCGGCGGCGGCAGCCAGGAGCAGCTGG |
| | | TGGAGTCTGGAGGAGGACTGGTGCAGCCTGAGGGCAGCCTGACCCTGA |
| | | CATGCAAGGCCTCCGGCTTTACCATCTCTAACAATTACTATATGTGCTG |
| | | GGTGCGGCAGGCCCCAGGCAAGGGACTGGAGTGGATCGCCTGCATCTA |
| | | CGGCGGCATCTCTGGCAGGACATACTATGCCAGCTGGGCCAAGGGCCG |
| | | CTTCACCATCTCCAAGACATCCTCTACCACAGTGACCCTGCAGATGACC |
| | | TCTCTGACAGCCGCCGATACCGCCACATACTTTTGCGTGCGGGGCTATG |
| | | TGGGCACCAGCAATCTGTGGGGCCCTGGCACCCTGGTGACAGTGAGCTC |
| | | C |
| 202 | 11011 | QVTLRESGPALVKPTQTLTLTCTFSGFSLSTSGMSVGWIRQPPGKALEWLA |
| | | DIWWDDKKDYNPSLKSRLTISKDTSKNQVVLKVTNMDPADTATYYCARS |
| | | MITNWYFDVWGAGTTVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKRKDT |
| | | LMISRTPEVTCVVVSVSHEDPEVKFNWYVDGVEVHNAKTKPREEQYNSTY |
| | | RVVSVLTVLHQDWLNGKEYKCKVSNKALPAPIEKTISKAKGQPREPQVYV |
| | | YPPSRDELTKNQVSLTCLVKGFYPSDIAVEWESNGQPENNYKTTPPVLDSD |
| | | GSFALVSKLTVDKSRWQQGNVFSCSVMHEALHNHYTQKSLSLSPG |
| 203 | 11011 | CAGGTGACACTGAGGGAGAGCGGACCAGCCCTGGTGAAGCCAACCCAG |
| | | ACACTGACCCTGACATGCACCTTCTCCGGCTTTAGCCTGTCCACATCTGG |
| | | CATGTCTGTGGGCTGGATCAGACAGCCACCTGGCAAGGCCCTGGAGTG |
| | | GCTGGCCGACATCTGGTGGGACGATAAGAAGGATTACAACCCTAGCCT |
| | | GAAGTCCAGACTGACAATCTCTAAGGACACCAGCAAGAACCAGGTGGT |
| | | GCTGAAGGTGACCAATATGGACCCCGCCGATACAGCCACCTACTATTGT |
| | | GCCCGGTCCATGATTACTAACTGGTATTTTGATGTCTGGGGGGCAGGAA |
| | | CAACCGTGACCGTCTCTTCTGCTAGCACTAAGGGGCCTTCCGTGTTTCCA |
| | | CTGGCTCCCTCTAGTAAATCCACCTCTGGAGGCACAGCTGCACTGGGAT |
| | | GTCTGGTGAAGGATTACTTCCCTGAACCAGTCACAGTGAGTTGGAACTC |
| | | AGGGGCTCTGACAAGTGGAGTCCATACTTTTCCCGCAGTGCTGCAGTCA |
| | | AGCGGACTGTACTCCCTGTCCTCTGTGGTCACCGTGCCTAGTTCAAGCCT |
| | | GGGCACCCAGACATATATCTGCAACGTGAATCACAAGCCATCAAATAC |
| | | AAAAGTCGACAAGAAAGTGGAGCCCAAGAGCTGTGATAAAACTCATAC |
| | | CTGCCCACCTTGTCCGGCGCCAGAGGCTGCAGGAGGACCAAGCGTGTTC |
| | | CTGTTTCCACCCAAGCCTAAAGACACACTGATGATTTCCCGAACCCCCG |
| | | AAGTCACATGCGTGGTCGTGTCTGTGAGTCACGAGGACCCTGAAGTCAA |
| | | GTTCAACTGGTACGTGGATGGCGTCGAGGTGCATAATGCCAAGACTAA |
| | | ACCTAGGGAGGAACAGTACAACTCAACCTATCGCGTCGTGAGCGTCCTG |
| | | ACAGTGCTGCACCAGGATTGGCTGAACGGCAAAGAATATAAGTGCAAA |
| | | GTGAGCAATAAGGCCCTGCCCGCTCCTATCGAGAAAACCATTTCCAAGG |
| | | CTAAAGGGCAGCCTCGCGAACCACAGGTCTACGTGTATCCTCCAAGCCG |
| | | GGACGAGCTGACAAAGAACCAGGTCTCCCTGACTTGTCTGGTGAAAGG |
| | | GTTTTACCCTAGTGATATCGCTGTGGAGTGGGAATCAAATGGACAGCCA |
| | | GAGAACAATTATAAGACTACCCCCCCTGTGCTGGACAGTGATGGGTCAT |
| | | TCGCACTGGTCTCCAAGCTGACAGTGGACAAATCTCGGTGGCAGCAGG |
| | | GAAATGTCTTTTCATGTAGCGTGATGCATGAAGCACTGCACAACCATTA |
| | | CACCCAGAAGTCACTGTCACTGTCACCAGGA |
| 204 | 2871 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPCSRSTSESTAALGCLVKDYFPEPVT |
| | | VSWNSGALTSGVHTFPAVLQSSGLYSLSSVVTVPSSNFGTQTYTCNVDHKP |
| | | SNTKVDKTVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGK |
| 205 | 2871 | GAAGTGCAGCTGGTCCAGTCCGGAGCCGAGGTGAAGAAACCCGGCGAA |
| | | TCACTGCGAATCAGCTGCAAGGGCAGCGGCTACAGCTTCTCCACATATT |
| | | GGATTAGCTGGGTGCGGCAGATGCCTGGGAAGGGACTGGAGTGGATGG |
| | | GGAAAATCTACCCCGGAGATAGCTACACTAACTATTCTCCTAGTTTTCA |
| | | GGGCCAGGTGACCATCTCCGCCGACAAGTCAATTAGCACAGCTTATCTG |
| | | CAGTGGAGCTCCCTGAAAGCTAGCGATACCGCAATGTACTATTGTGCCA |
| | | GAGGCTACGGGATTTTCGACTATTGGGGACAGGGCACACTGGTCACTGT |
| | | GTCTAGTGCTTCTACAAAGGGGCCCAGTGTGTTTCCACTGGCACCCTGC |
| | | TCCAGGTCTACCAGTGAATCAACAGCCGCTCTGGGATGTCTGGTGAAAG |
| | | ATTACTTCCCAGAGCCCGTCACTGTGAGCTGGAACTCCGGCGCACTGAC |
| | | ATCTGGGGTCCACACTTTTCCTGCCGTGCTGCAGTCAAGCGGCCTGTAC |
| | | AGCCTGTCCTCTGTGGTCACCGTGCCAAGTTCAAATTTCGGGACTCAGA |
| | | CCTATACATGCAACGTGGACCACAAGCCTTCTAATACCAAGGTCGATAA |
| | | AACAGTGGAACCAAAGAGTTGTGACAAAACTCATACCTGCCCCCCTTGT |
| | | CCTGCTCCAGAGCTGCTGGGAGGACCATCCGTGTTCCTGTTTCCACCCA |
| | | AGCCCAAAGATACACTGATGATCAGCCGCACTCCAGAAGTGACCTGCG |
| | | TGGTCGTGGACGTGTCCCACGAGGACCCCGAAGTCAAGTTTAACTGGTA |
| | | CGTGGACGGCGTCGAGGTGCATAATGCCAAGACCAAACCACGGGAGGA |
| | | ACAGTACAATTCAACATATAGAGTCGTGAGCGTCCTGACTGTGCTGCAC |
| | | CAGGATTGGCTGAACGGCAAGGAGTATAAGTGCAAAGTGTCTAATAAG |
| | | GCACTGCCCGCCCCTATCGAGAAAACCATTAGCAAGGCAAAAGGGCAG |
| | | CCTAGGGAACCACAGGTCTACGTGTATCCTCCAAGCCGCGATGAGCTGA |
| | | CTAAGAACCAGGTCTCCCTGACCTGTCTGGTGAAAGGGTTCTACCCCAG |
| | | TGACATTGCCGTGGAGTGGGAATCAAATGGACAGCCTGAAAACAATTA |
| | | TAAGACCACACCCCCTGTGCTGGACTCTGATGGAAGTTTCGCCCTGGTC |
| | | TCCAAGCTGACTGTGGACAAATCTCGATGGCAGCAGGGCAACGTCTTTA |
| | | GCTGTTCCGTGATGCATGAGGCTCTGCACAATCATTACACCCAGAAGTC |
| | | TCTGAGTCTGTCACCTGGCAAA |
| 206 | 2872 | SYELTQPPSVSVSPGQTASITCSGDNIGDQYAHWYQQKPGQSPVLVIYQDK |
| | | NRPSGIPERFSGSNSGNTATLTISGTQAMDEADYYCATYTGFGSLAVFGGG |
| | | TKLTVLGQPKAAPSVTLFPPSSEELQANKATLYCLISDFYPGAVTVAWKAD |
| | | SSPVKAGVETTTPSKQSNNKYAASSYLSITPEQWKSHRSYSCQVTHEGSTV |
| | | EKTVAPTECS |
| 207 | 2872 | TCCTATGAACTGACCCAGCCCCCTTCCGTGTCTGTCAGTCCAGGGCAGA |
| | | CAGCCTCCATCACTTGCTCTGGAGACAACATTGGCGATCAGTACGCTCA |
| | | CTGGTATCAGCAGAAGCCTGGCCAGTCTCCAGTGCTGGTCATCTACCAG |
| | | GACAAAAATCGGCCCAGTGGGATTCCTGAGAGATTCTCAGGGAGCAAC |
| | | TCCGGAAATACCGCCACACTGACTATCAGCGGGACACAGGCAATGGAC |
| | | GAAGCCGATTACTATTGCGCTACCTATACAGGCTTCGGGTCCCTGGCAG |
| | | TGTTTGGCGGGGGAACTAAGCTGACCGTGCTGGGACAGCCAAAAGCCG |
| | | CTCCCTCTGTGACCCTGTTCCCACCCAGCTCCGAGGAACTGCAGGCTAA |
| | | CAAGGCAACCCTGTACTGTCTGATCAGCGACTTTTATCCTGGAGCAGTG |
| | | ACAGTCGCCTGGAAGGCTGATTCTAGTCCTGTGAAAGCTGGCGTCGAGA |
| | | CCACAACTCCATCAAAGCAGAGCAACAACAAGTACGCAGCCTCAAGCT |
| | | ATCTGAGTATTACTCCCGAACAGTGGAAGTCACACAGGTCTTACAGTTG |
| | | CCAGGTGACCCATGAGGGCAGCACCGTGGAAAAAACAGTCGCCCCTAC |
| | | TGAGTGTTCC |
| 208 | 11074 | DIQMTQSPSTLSASVGDRVTITCKCQLSVGYMHWYQQKPGKAPKLLIYDTS |
| | | KLASGVPSRFSGSGSGTEFTLTISSLQPDDFATYYCFQGSGYPFTFGGGTKLE |
| | | IKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTK |
| | | SFNRGEC |
| 209 | 11074 | GATATTCAGATGACCCAGTCTCCCAGCACACTGTCCGCCTCTGTGGGCG |
| | | ACCGGGTGACCATCACATGCAAGTGTCAGCTGAGCGTGGGCTACATGC |
| | | ACTGGTATCAGCAGAAGCCCGGCAAGGCCCCTAAGCTGCTGATCTACG |
| | | ATACCAGCAAGCTGGCCTCCGGCGTGCCATCTAGATTCAGCGGCTCCGG |
| | | CTCTGGCACCGAGTTTACCCTGACAATCAGCTCCCTGCAGCCCGACGAT |
| | | TTCGCCACATACTATTGCTTTCAGGGGAGCGGCTACCCATTCACATTCG |
| | | GAGGGGGAACTAAACTGGAAATCAAGAGGACCGTCGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTCAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 210 | 11082 | QVTLRESGPALVKPTQTLTLTCTFSGFSLSTSGMSVGWIRQPPGKALEWLA |
| | | DIWWDDKKDYNPSLKSRLTISKDTSKNQVVLKVTNMDPADTATYYCARS |
| | | MITNWYFDVWGAGTTVTVSSVEGGSGGSGGSGGSGGVDDIQMTQSPSTLS |
| | | ASVGDRVTITCKCQLSVGYMHWYQQKPGKAPKLLIYDTSKLASGVPSRFS |
| | | GSGSGTEFTLTISSLQPDDFATYYCFQGSGYPFTFGGGTKLEIKAAEPKSSDK |
| | | THTCPPCPAPEAAGGPSVFLFPPKPKDTLMISRTPEVTCVVVSVSHEDPEVK |
| | | FNWYVDGVEVHNAKTKPREEQYNSTYRVVSVLTVLHQDWLNGKEYKCK |
| | | VSNKALPAPIEKTISKAKGQPREPQVYVLPPSRDELTKNQVSLLCLVKGFYP |
| | | SDIAVEWESNGQPENNYLTWPPVLDSDGSFFLYSKLTVDKSRWQQGNVFS |
| | | CSVMHEALHNHYTQKSLSLSPG |
| 211 | 11082 | CAGGTGACCCTGAGAGAGAGCGGACCCGCCCTGGTGAAGCCTACCCAG |
| | | ACACTGACCCTGACATGCACCTTCAGCGGCTTTAGCCTGTCCACCTCTG |
| | | GCATGTCCGTGGGATGGATCAGGCAGCCACCTGGCAAGGCCCTGGAGT |
| | | GGCTGGCCGACATCTGGTGGGACGATAAGAAGGATTACAACCCTTCCCT |
| | | GAAGTCTCGCCTGACAATCTCCAAGGACACCTCTAAGAACCAGGTGGTG |
| | | CTGAAGGTGACCAATATGGACCCAGCCGATACAGCCACCTACTATTGTG |
| | | CCCGGTCCATGATCACAAATTGGTATTTCGACGTGTGGGGAGCCGGAAC |
| | | CACAGTGACCGTGAGCTCCGTGGAGGGAGGCAGCGGAGGCTCCGGAGG |
| | | CTCTGGAGGCAGCGGAGGAGTGGACGATATCCAGATGACACAGAGCCC |
| | | CTCCACCCTGTCTGCCAGCGTGGGCGACCGGGTGACAATCACCTGCAAG |
| | | TGTCAGCTGTCCGTGGGCTACATGCACTGGTATCAGCAGAAGCCTGGCA |
| | | AGGCCCCAAAGCTGCTGATCTACGATACCAGCAAGCTGGCCTCCGGCGT |
| | | GCCTTCTAGGTTCTCCGGCTCTGGCAGCGGCACAGAGTTTACACTGACC |
| | | ATCTCTAGCCTGCAGCCAGACGATTTCGCCACCTACTATTGCTTTCAGG |
| | | GCAGCGGCTATCCCTTCACATTTGGCGGCGGCACCAAGCTGGAGATCAA |
| | | GGCCGCCGAGCCTAAGTCCTCTGACAAGACACACACCTGCCCACCCTGT |
| | | CCGGCGCCAGAGGCAGCAGGAGGACCAAGCGTGTTCCTGTTTCCACCC |
| | | AAGCCCAAAGACACCCTGATGATTAGCCGAACCCCTGAAGTCACATGC |
| | | GTGGTCGTGTCCGTGTCTCACGAGGACCCAGAAGTCAAGTTCAACTGGT |
| | | ACGTGGATGGCGTCGAGGTGCATAATGCCAAGACAAAACCCCGGGAGG |
| | | AACAGTACAACAGCACCTATAGAGTCGTGTCCGTCCTGACAGTGCTGCA |
| | | CCAGGATTGGCTGAACGGCAAGGAATATAAGTGCAAAGTGTCCAATAA |
| | | GGCCCTGCCCGCTCCTATCGAGAAAACCATTTCTAAGGCAAAAGGCCAG |
| | | CCTCGCGAACCACAGGTCTACGTGCTGCCTCCATCCCGGGACGAGCTGA |
| | | CAAAGAACCAGGTCTCTCTGCTGTGCCTGGTGAAAGGCTTCTATCCATC |
| | | AGATATTGCTGTGGAGTGGGAAAGCAATGGGCAGCCCGAGAACAATTA |
| | | CCTGACTTGGCCCCCTGTGCTGGACTCTGATGGGAGTTTCTTTCTGTATT |
| | | CTAAGCTGACCGTGGATAAAAGTAGGTGGCAGCAGGGAAATGTCTTTA |
| | | GTTGTTCAGTGATGCATGAAGCCCTGCATAACCACTACACCCAGAAAAG |
| | | CCTGTCCCTGTCCCCCGGA |
| 212 | 8021 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLIMSRTPE |
| | | VTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPG |
| 213 | 8021 | GAGGTCCAGCTGGTGCAGAGTGGCGCTGAGGTCAAGAAACCAGGGGAA |
| | | AGTCTGCGGATCTCATGCAAGGGCAGCGGGTACTCTTTCAGTACTTATT |
| | | GGATTTCTTGGGTGAGACAGATGCCCGGAAAGGGCCTGGAGTGGATGG |
| | | GGAAAATCTACCCCGGAGACTCCTACACCAACTATTCACCTAGCTTTCA |
| | | GGGCCAGGTCACCATCTCTGCAGACAAGTCCATTTCTACAGCCTATCTG |
| | | CAGTGGAGCTCCCTGAAAGCCAGCGATACAGCTATGTACTATTGTGCAA |
| | | GAGGATACGGCATTTTCGATTATTGGGGCCAGGGCACCCTGGTCACCGT |
| | | CTCATCTGCTAGCACTAAGGGGCCTTCCGTGTTTCCACTGGCTCCCTCTA |
| | | GTAAATCCACCTCTGGAGGCACAGCTGCACTGGGATGTCTGGTGAAGG |
| | | ATTACTTCCCTGAACCAGTCACAGTGAGTTGGAACTCAGGGGCTCTGAC |
| | | AAGTGGAGTCCATACTTTTCCCGCAGTGCTGCAGTCAAGCGGACTGTAC |
| | | TCCCTGTCCTCTGTGGTCACCGTGCCTAGTTCAAGCCTGGGCACCCAGA |
| | | CATATATCTGCAACGTGAATCACAAGCCATCAAATACAAAAGTCGACA |
| | | AGAAAGTGGAGCCCAAGAGCTGTGATAAAACTCATACCTGCCCACCTT |
| | | GTCCGGCGCCAGAACTGCTGGGAGGACCAAGCGTGTTCCTGTTTCCACC |
| | | CAAGCCTAAAGACACCCTGATGATTTCCCGGACTCCTGAGGTCACCTGC |
| | | GTGGTCGTGGACGTGTCTCACGAGGACCCCGAAGTCAAGTTCAACTGGT |
| | | ACGTGGATGGCGTCGAAGTGCATAATGCCAAGACCAAACCCCGGGAGG |
| | | AACAGTACAACTCTACCTATAGAGTCGTGAGTGTCCTGACAGTGCTGCA |
| | | CCAGGACTGGCTGAATGGGAAGGAGTATAAGTGTAAAGTGAGCAACAA |
| | | AGCCCTGCCCGCCCCAATCGAAAAAACAATCTCTAAAGCAAAAGGACA |
| | | GCCTCGCGAACCACAGGTCTACGTCTACCCCCCATCAAGAGATGAACTG |
| | | ACAAAAAATCAGGTCTCTCTGACATGCCTGGTCAAAGGATTCTACCCTT |
| | | CCGACATCGCCGTGGAGTGGGAAAGTAACGGCCAGCCCGAGAACAATT |
| | | ACAAGACCACACCCCCTGTCCTGGACTCTGATGGGAGTTTCGCTCTGGT |
| | | GTCAAAGCTGACCGTCGATAAAAGCCGGTGGCAGCAGGGCAATGTGTT |
| | | TAGCTGCTCCGTCATGCACGAAGCCCTGCACAATCACTACACACAGAAG |
| | | TCCCTGAGCCTGAGCCCTGGC |
| 214 | 8022 | SYELTQPPSVSVSPGQTASITCSGDNIGDQYAHWYQQKPGQSPVLVIYQDK |
| | | NRPSGIPERFSGSNSGNTATLTISGTQAMDEADYYCATYTGFGSLAVFGGG |
| | | TKLTVLGQPKAAPSVTLFPPSSEELQANKATLVCLISDFYPGAVTVAWKAD |
| | | SSPVKAGVETTTPSKQSNNKYAASSYLSLTPEQWKSHRSYSCQVTHEGSTV |
| | | EKTVAPTECS |
| 215 | 8022 | AGCTATGAACTGACACAGCCACCTAGCGTGTCCGTCTCTCCAGGACAGA |
| | | CTGCATCCATCACCTGCTCTGGCGACAACATTGGGGATCAGTACGCCCA |
| | | CTGGTATCAGCAGAAGCCAGGGCAGAGTCCCGTGCTGGTCATCTACCAG |
| | | GACAAAAATCGGCCTTCCGGCATCCCCGAGAGATTCAGTGGATCAAAC |
| | | AGCGGCAATACCGCTACACTGACTATCAGCGGCACCCAGGCCATGGAC |
| | | GAAGCTGATTACTATTGTGCCACCTATACAGGGTTTGGAAGTCTGGCCG |
| | | TCTTCGGCGGCGGCACCAAACTGACCGTCCTGGGGCAGCCAAAAGCGG |
| | | CGCCCAGTGTCACACTGTTTCCCCCTAGCTCCGAGGAACTGCAGGCTAA |
| | | CAAAGCAACACTGGTGTGTCTGATCAGCGACTTCTACCCTGGAGCTGTG |
| | | ACTGTCGCCTGGAAGGCTGATTCTAGTCCAGTGAAAGCAGGCGTCGAG |
| | | ACCACAACTCCCTCTAAGCAGAGTAACAACAAGTACGCAGCCTCAAGC |
| | | TATCTGTCACTGACCCCAGAACAGTGGAAGAGCCACCGGAGCTATTCCT |
| | | GCCAGGTCACTCACGAAGGCTCCACTGTCGAGAAAACCGTCGCTCCCAC |
| | | CGAATGTTCA |
| 216 | 14395 | QVQLQQSGPELVKPGASVKISCKASGYVFSSYWMNWVKQRPGKGLEWIG |
| | | RIYPGNGDTNYNGKFKDKATLTADKFSSTAYMQLSNLTSEDSAVYFCASY |
| | | YELDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTIPPVLDSDGSFFLY |
| | | SKLTVDKSRWQQGNVFSCSVMHEALHNHYTQKSLSLSPG |
| 217 | 14395 | CAGGTGCAGCTGCAGCAGTCCGGACCAGAGCTGGTGAAGCCTGGGGCC |
| | | AGCGTGAAGATCAGCTGCAAGGCCTCCGGCTACGTGTTTAGCTCCTATT |
| | | GGATGAACTGGGTGAAGCAGCGGCCAGGCAAGGGACTGGAGTGGATCG |
| | | GCAGAATCTACCCCGGCAATGGCGACACAAACTATAATGGCAAGTTCA |
| | | AGGACAAGGCCACCCTGACAGCCGATAAGTTTTCTAGCACCGCCTACAT |
| | | GCAGCTGTCTAACCTGACAAGCGAGGACTCCGCCGTGTACTTCTGTGCC |
| | | TCTTACTATGAGCTGGATTATTGGGGCCAGGGCACCAGCGTGACAGTGT |
| | | CCTCTGCTAGCACAAAGGGCCCCTCCGTGTTTCCTCTGGCCCCATCCTCT |
| | | AAGTCCACCTCTGGAGGAACAGCCGCCCTGGGCTGTCTGGTGAAGGATT |
| | | ACTTCCCTGAGCCAGTGACCGTGTCCTGGAACTCTGGGGCCCTGACCAG |
| | | CGGAGTGCACACATTTCCCGCCGTGCRTCAGAGCTCCGGACTGTACTCC |
| | | CTGTCTAGCGTGGTGACCGTGCCTTCCTCTAGCCTGGGCACCCAGACAT |
| | | ATATCTGCAACGTGAATCACAAGCCTTCCAATACAAAGGTCGACAAGA |
| | | AGGTGGAGCCAAAGTCTTGTGATAAGACCCACACATGCCCACCTTGTCC |
| | | GGCGCCAGAGGCCGCCGGAGGACCAAGCGTGTTCCTGTTTCCACCCAA |
| | | GCCCAAGGACACCCTGATGATCTCCCGGACCCCAGAGGTGACATGCGT |
| | | GGTGGTGAGCGTGTCCCACGAGGACCCCGAGGTGAAGTTTAACTGGTA |
| | | CGTGGATGGCGTGGAGGTGCACAATGCCAAGACAAAGCCCCGGGAGGA |
| | | GCAGTACAATTCTACCTATAGAGTGGTGAGCGTGCTGACAGTGCTGCAC |
| | | CAGGATTGGCTGAACGGCAAGGAGTATAAGTGTAAGGTGAGCAATAAG |
| | | GCCCTGCCAGCCCCCATCGAGAAGACCATCTCCAAGGCCAAGGGCCAG |
| | | CCTCGCGAACCACAGGTGTACACTCTGCCTCCATCTCGGGACGAGCTGA |
| | | CTAAGAACCAGGTCAGTCTGACCTGTCTGGTGAAAGGATTCTATCCCAG |
| | | CGATATCGCTGTGGAGTGGGAATCCAATGGCCAGCCTGAGAACAATTA |
| | | CAAGACCACACCCCCTGTGCTGGACTCTGATGGCAGTTTCTTTCTGTATA |
| | | GTAAGCTGACCGTCGATAAATCACGATGGCAGCAGGGGAACGTGTTCA |
| | | GCTGTTCAGTGATGCACGAAGCCCTGCACAACCATTACACCCAGAAGA |
| | | GCCTGAGCCTGTCTCCCGGC |
| 218 | 1380 | EPKSSDKTHTCPPCPAPEAAGGPSVFLFPPKPKDTLMISRTPEVTCVVVSVS |
| | | HEDPEVKFNWYVDGVEVHNAKTKPREEQYNSTYRVVSVLTVLHQDWLN |
| | | GKEYKCKVSNKALPAPIEKTISKAKGQPREPQVYVYPPSRDELTKNQVSLT |
| | | CLVKGFYPSDIAVEWESNGQPENNYKTTPPVLDSDGSFALVSKLTVDKSR |
| | | WQQGNVFSCSVMHEALHNHYTQKSLSLSPGK |
| 219 | 1380 | GAACCTAAGAGCAGCGACAAGACTCACACCTGCCCACCTTGTCCAGCA |
| | | CCAGAAGCCGCCGGAGGACCAAGCGTGTTCCTGTTTCCACCCAAGCCCA |
| | | AAGATACCCTGATGATCAGCCGAACACCCGAAGTGACTTGCGTGGTCGT |
| | | GAGCGTGTCCCACGAGGACCCCGAAGTCAAGTTCAACTGGTACGTGGA |
| | | CGGCGTCGAAGTGCATAATGCTAAGACAAAACCACGGGAGGAACAGTA |
| | | CAACTCTACTTATAGAGTCGTGAGTGTCCTGACCGTGCTGCATCAGGAT |
| | | TGGCTGAACGGCAAAGAGTATAAGTGCAAAGTGTCTAATAAGGCCCTG |
| | | CCTGCTCCAATCGAGAAAACCATTAGTAAGGCTAAAGGGCAGCCCAGG |
| | | GAACCTCAGGTCTACGTGTATCCTCCAAGTCGCGACGAGCTGACCAAGA |
| | | ACCAGGTCTCACTGACATGTCTGGTGAAAGGATTTTACCCTTCCGATAT |
| | | TGCAGTGGAGTGGGAATCTAATGGCCAGCCAGAGAACAATTATAAGAC |
| | | CACACCCCCTGTGCTGGACAGCGATGGGTCCTTCGCACTGGTCTCAAAG |
| | | CTGACAGTGGACAAAAGCAGATGGCAGCAGGGAAACGTCTTTAGCTGT |
| | | TCCGTGATGCACGAAGCCCTGCACAATCATTACACTCAGAAGTCTCTGA |
| | | GTCTGTCACCTGGCAAA |
| 220 | 8056 | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLN |
| | | GKEYKCKVSNKALPAPIEKTISKAKGQPREPQVYVYPPSRDELTKNQVSLT |
| | | CLVKGFYPSDIAVEWESNGQPENNYKTTPPVLDSDGSFALVSKLTVDKSR |
| | | WQQGNVFSCSVMHEALHNHYTQKSLSLSPG |
| 221 | 8056 | GAACCTAAATCATCCGATAAAACTCACACCTGTCCACCCTGTCCGGCGC |
| | | CAGAACTGCTGGGGGGGCCCTCCGTCTTCCTGTTTCCCCCTAAGCCAAA |
| | | AGACACCCTGATGATCTCTAGGACTCCCGAAGTCACCTGCGTGGTCGTG |
| | | GACGTGAGTCACGAGGACCCCGAAGTCAAGTTCAACTGGTACGTGGAT |
| | | GGCGTCGAGGTGCATAATGCCAAGACCAAACCCCGGGAGGAACAGTAC |
| | | AACAGCACCTATAGAGTCGTGTCCGTCCTGACAGTGCTGCACCAGGACT |
| | | GGCTGAACGGAAAGGAGTATAAGTGCAAAGTGTCAAATAAGGCCCTGC |
| | | CCGCTCCTATCGAGAAAACCATTAGCAAGGCTAAAGGCCAGCCTCGCG |
| | | AACCCCAGGTCTACGTGTATCCCCCTAGCCGCGACGAGCTGACAAAGA |
| | | ACCAGGTCTCCCTGACTTGTCTGGTGAAAGGGTTTTACCCTAGTGATAT |
| | | CGCAGTGGAGTGGGAATCAAATGGACAGCCAGAAAACAATTATAAGAC |
| | | CACACCACCCGTGCTGGACAGCGATGGCTCCTTCGCACTGGTCTCCAAG |
| | | CTGACTGTGGATAAATCTCGATGGCAGCAGGGGAACGTCTTTAGCTGCT |
| | | CCGTGATGCACGAGGCCCTGCACAATCATTACACACAGAAGTCTCTGAG |
| | | CTTAAGCCCTGGA |
| 222 | 10619 | QVQLVQSGAEVKKPGASVRVSCRASGYIFTESGITWVRQAPGQGLEWMG |
| | | WISGYSGDTKYAQKLQGRVTMTKDTSTTTAYMELRSLRYDDTAVYYCAR |
| | | DVQYSGSYLGAYYFDYWSPGTLVTVSSASTKGPSVFPLAPSSKSTSGGTAA |
| | | LGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPEAAGGPSVFLFP |
| | | PKPKDTLMISRTPEVTCVVVSVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYVYPPSRDELTKNQVSLTCLVKGFYPSDIAVEWESNGQPENNYKTTP |
| | | PVLDSDGSFALVSKLTVDKSRWQQGNVFSCSVMHEALHNHYTQKSLSLSP |
| | | G |
| 223 | 10619 | CAGGTGCAGCTGGTCCAGTCCGGGGCTGAAGTGAAAAAACCTGGGGCA |
| | | TCCGTGCGGGTGTCATGTCGGGCAAGCGGGTATATCTTTACTGAGTCTG |
| | | GAATCACCTGGGTGAGGCAGGCTCCCGGACAGGGACTGGAATGGATGG |
| | | GATGGATTTCTGGATACAGTGGCGACACAAAGTATGCACAGAAACTGC |
| | | AGGGCCGCGTCACCATGACAAAGGATACTTCAACCACAACTGCCTACAT |
| | | GGAGCTGCGGAGCCTGAGATATGACGATACAGCCGTGTACTATTGCGCC |
| | | CGGGACGTGCAGTACAGCGGGTCCTACCTGGGGGCATACTACTTCGATT |
| | | ACTGGTCACCTGGAACTCTGGTCACCGTCTCTTCAGCTAGCACTAAGGG |
| | | GCCTTCCGTGTTTCCACTGGCTCCCTCTAGTAAATCCACCTCTGGAGGCA |
| | | CAGCTGCACTGGGATGTCTGGTGAAGGATTACTTCCCTGAACCAGTCAC |
| | | AGTGAGTTGGAACTCAGGGGCTCTGACAAGTGGAGTCCATACTTTTCCC |
| | | GCAGTGCTGCAGTCAAGCGGACTGTACTCCCTGTCCTCTGTGGTCACCG |
| | | TGCCTAGTTCAAGCCTGGGCACCCAGACATATATCTGCAACGTGAATCA |
| | | CAAGCCATCAAATACAAAAGTCGACAAGAAAGTGGAGCCCAAGAGCTG |
| | | TGATAAAACTCATACCTGCCCACCTTGTCCGGCGCCAGAGGCTGCAGGA |
| | | GGACCAAGCGTGTCCCTGTTTCCACCCAAGCCTAAAGACACACTGATGA |
| | | TTTCCCGAACCCCCGAAGTCACATGCGTGGTCGTGTCTGTGAGTCACGA |
| | | GGACCCTGAAGTCAAGTTCAACTGGTACGTGGATGGCGTCGAGGTGCAT |
| | | AATGCCAAGACTAAACCTAGGGAGGAACAGTACAACTCAACCTATCGC |
| | | GTCGTGAGCGTCCTGACAGTGCTGCACCAGGATTGGCTGAACGGCAAA |
| | | GAATATAAGTGCAAAGTGAGCAATAAGGCCCTGCCCGCTCCTATCGAG |
| | | AAAACCATTTCCAAGGCTAAAGGGCAGCCTCGCGAACCACAGGTCTAC |
| | | GTGTATCCTCCAAGCCGGGACGAGCTGACAAAGAACCAGGTCTCCCTG |
| | | ACTTGTCTGGTGAAAGGGTTTTACCCTAGTGATATCGCTGTGGAGTGGG |
| | | AATCAAATGGACAGCCAGAGAACAATTATAAGACTACCCCCCCTGTGCT |
| | | GGACAGTGATGGGTCATTCGCACTGGTCTCCAAGCTGACAGTGGACAA |
| | | ATCTCGGTGGCAGCAGGGAAATGTCTTTTCATGTAGCGTGATGCATGAA |
| | | GCACTGCACAACCATTACACCCAGAAGTCACTGTCACTGTCACCAGGA |
| 224 | 10620 | QVQLVQSGAEVKKPGASVRVSCRASGYIFTESGITWVRQAPGQGLEWMG |
| | | WISGYSGDTKYAQKLQGRVTMTKDTSTTTAYMELRSLRYDDTAVYYCAR |
| | | DVQYSGSYLGAYYFDYWSPGTLVTVSSASTKGPSVFPLAPSSKSTSGGTAA |
| | | LGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPEAAGGPSVFLFP |
| | | PKPKDTLMISRTPEVTCVVVSVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYVLPPSRDELTKNQVSLLCLVKGFYPSDIAVEWESNGQPENNYLTWP |
| | | PVLDSDGSFFLYSKLTVDKSRWQQGNVFSCSVMHEALHNHYTQKSLSLSP |
| | | G |
| 225 | 10620 | CAGGTGCAGCTGGTCCAGTCCGGGGCTGAAGTGAAAAAACCTGGGGCA |
| | | TCCGTGCGGGTGTCATGTCGGGCAAGCGGGTATATCTTTACTGAGTCTG |
| | | GAATCACCTGGGTGAGGCAGGCTCCCGGACAGGGACTGGAATGGATGG |
| | | GATGGATTTCTGGATACAGTGGCGACACAAAGTATGCACAGAAACTGC |
| | | AGGGCCGCGTCACCATGACAAAGGATACTTCAACCACAACTGCCTACAT |
| | | GGAGCTGCGGAGCCTGAGATATGACGATACAGCCGTGTACTATTGCGCC |
| | | CGGGACGTGCAGTACAGCGGGTCCTACCTGGGGGCATACTACTTCGATT |
| | | ACTGGTCACCTGGAACTCTGGTCACCGTCTCTTCAGCTAGCACTAAGGG |
| | | GCCTTCCGTGTTTCCACTGGCTCCCTCTAGTAAATCCACCTCTGGAGGCA |
| | | CAGCTGCACTGGGATGTCTGGTGAAGGATTACTTCCCTGAACCAGTCAC |
| | | AGTGAGTTGGAACTCAGGGGCTCTGACAAGTGGAGTCCATACTTTTCCC |
| | | GCAGTGCTGCAGTCAAGCGGACTGTACTCCCTGTCCTCTGTGGTCACCG |
| | | TGCCTAGTTCAAGCCTGGGCACCCAGACATATATCTGCAACGTGAATCA |
| | | CAAGCCATCAAATACAAAAGTCGACAAGAAAGTGGAGCCCAAGAGCTG |
| | | TGATAAAACTCATACCTGCCCACCTTGTCCGGCGCCAGAGGCAGCAGGA |
| | | GGACCAAGCGTGTTCCTGTTTCCACCCAAGCCCAAAGACACCCTGATGA |
| | | TTAGCCGAACCCCTGAAGTCACATGCGTGGTCGTGTCCGTGTCTCACGA |
| | | GGACCCAGAAGTCAAGTTCAACTGGTACGTGGATGGCGTCGAGGTGCA |
| | | TAATGCCAAGACAAAACCCCGGGAGGAACAGTACAACAGCACCTATAG |
| | | AGTCGTGTCCGTCCTGACAGTGCTGCACCAGGATTGGCTGAACGGCAAG |
| | | GAATATAAGTGCAAAGTGTCCAATAAGGCCCTGCCCGCTCCTATCGAGA |
| | | AAACCATTTCTAAGGCAAAAGGCCAGCCTCGCGAACCACAGGTCTACG |
| | | TGCTGCCTCCATCCCGGGACGAGCTGACAAAGAACCAGGTCTCTCTGCT |
| | | GTGCCTGGTGAAAGGCTTCTATCCATCAGATATTGCTGTGGAGTGGGAA |
| | | AGCAATGGGCAGCCCGAGAACAATTACCTGACTTGGCCCCCTGTGCTGG |
| | | ACTCTGATGGGAGTTTCTTTCTGTATTCTAAGCTGACCGTGGATAAAAG |
| | | TAGGTGGCAGCAGGGAAATGTCTTTAGTTGTTCAGTGATGCATGAAGCC |
| | | CTGCATAACCACTACACCCAGAAAAGCCTGTCCCTGTCCCCCGGA |
| 226 | 11150 | DIQMTQSPSSLSASVGDRVTITCRASQDVNTAVAWYQQKPGKAPKLLIYSA |
| | | SFLYSGVPSRFSGSRSGTDFTLTISSLQPEDFATYYCQQHYTTPPTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 227 | 11150 | GACATCCAGATGACACAGTCCCCAAGCTCCCTGTCCGCCTCTGTGGGCG |
| | | ACAGGGTGACCATCACATGCCGCGCCTCTCAGGATGTGAACACCGCCGT |
| | | GGCCTGGTACCAGCAGAAGCCAGGCAAGGCCCCCAAGCTGCTGATCTA |
| | | CAGCGCCTCCTTCCTGTATTCTGGCGTGCCCAGCCGGTTTTCTGGCAGCA |
| | | GATCCGGCACCGACTTCACCCTGACAATCTCTAGCCTGCAGCCTGAGGA |
| | | TTTTGCCACATACTATTGTCAGCAGCACTATACCACACCCCCTACCTTCG |
| | | GCCAGGGCACAAAGGTGGAGATCAAGCGGACAGTGGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTCAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 228 | 20891 | DIQMTQSPSSLSASVGDRVTITCSASQGISNYLNWYQQKPGKAPKLLIYYTS |
| | | SLHSGVPSRFSGSGSGTDFTFTISSLQPEDIATYYCQQYSKFPWTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 229 | 20891 | GACATCCAGATGACACAGAGCCCAAGCTCCCTGAGCGCCTCCGTGGGC |
| | | GATAGAGTGACCATCACATGCTCTGCCAGCCAGGGCATCTCCAACTACC |
| | | TGAATTGGTATCAGCAGAAGCCCGGCAAGGCCCCTAAGCTGCTGATCTA |
| | | CTATACCTCTAGCCTGCACTCCGGCGTGCCTTCTAGGTTTTCCGGCTCTG |
| | | GCAGCGGCACCGACTTCACCTTTACAATCTCCTCTCTGCAGCCTGAGGA |
| | | TATCGCCACATACTATTGTCAGCAGTACTCTAAGTTCCCATGGACCTTTG |
| | | GCCAGGGCACAAAGGTGGAGATCAAGCGCACAGTGGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTCAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 230 | 20892 | DIQMTQSPSSLSASVGDRVTITCSASQGISNYLNWYQQKPGKAPKLLIYYTS |
| | | SLHSGVPSRFSGSGSGTDYTLTISSLQPEDIATYYCQQYSKFPWTFGQGTKL |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 231 | 20892 | GACATCCAGATGACACAGAGCCCAAGCTCCCTGAGCGCCTCCGTGGGC |
| | | GATAGAGTGACCATCACATGCTCTGCCAGCCAGGGCATCTCCAACTACC |
| | | TGAATTGGTATCAGCAGAAGCCCGGCAAGGCCCCTAAGCTGCTGATCTA |
| | | CTATACCTCTAGCCTGCACTCCGGCGTGCCTTCTAGGTTTTCCGGCTCTG |
| | | GCAGCGGAACCGACTACACCCTGACAATCTCCTCTCTGCAGCCTGAGGA |
| | | TATCGCCACATACTATTGTCAGCAGTATTCTAAGTTCCCATGGACCTTTG |
| | | GCCAGGGCACAAAGCTGGAGATCAAGCGCACAGTGGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTCAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 232 | 20893 | DIQMTQSPSSLSASVGDRVTITCSASQGISNYLNWYQQKPGKTVKLLIYYTS |
| | | SLHSGVPSRFSGSGSGTDYTLTISSLQPEDIATYYCQQYSKFPWTFGQGTKL |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVIHQGLSSPVT |
| | | KSFNRGEC |
| 233 | 20893 | GACATCCAGATGACACAGAGCCCTAGCTCCCTGAGCGCCTCCGTGGGCG |
| | | ATAGAGTGACCATCACATGCTCTGCCAGCCAGGGCATCTCCAACTACCT |
| | | GAATTGGTATCAGCAGAAGCCTGGCAAGACCGTGAAGCTGCTGATCTA |
| | | CTATACATCTAGCCTGCACTCCGGCGTGCCATCTAGGTTTTCCGGCTCTG |
| | | GCAGCGGAACCGACTACACCCTGACAATCTCCTCTCTGCAGCCAGAGGA |
| | | TATCGCCACATACTATTGTCAGCAGTATTCTAAGTTCCCCTGGACCTTTG |
| | | GCCAGGGCACAAAGCTGGAGATCAAGCGCACCGTGGCGGCGCCCAGTG |
| | | TCTTCATTTTTCCCCCTAGCGACGAACAGCTGAAGTCTGGGACAGCCAG |
| | | TGTGGTCTGTCTGCTGAACAACTTCTACCCTAGAGAGGCTAAAGTGCAG |
| | | TGGAAGGTCGATAACGCACTGCAGTCCGGAAATTCTCAGGAGAGTGTG |
| | | ACTGAACAGGACTCAAAAGATAGCACCTATTCCCTGTCAAGCACACTGA |
| | | CTCTGAGCAAGGCCGACTACGAGAAGCATAAAGTGTATGCTTGTGAAG |
| | | TCACCCACCAGGGGCTGAGTTCACCAGTCACAAAATCATTCAACAGAG |
| | | GGGAGTGC |
| 234 | 20894 | EVQLVESGGGLVQPGGSLRLSCAASGFTFSEFGMHWVRQAPGKGLEWVSY |
| | | ISSGGSTIYYADSVKGRFTISRDNAKNSLYLQMNSLRAEDTAVYYCARDW |
| | | VDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNXALPAPIEKTISKAKGQPREPQVYTLPPSRDEL |
| | | TKNQVSLTCLVKGFYPSDIAVEWESNGQPENNYKTTPPVLDSDGSFFLYSK |
| | | LTVDKSRWQQGNVFSCSVMHEALHNHYTQKSLSLSPG |
| 235 | 20894 | GAGGTGCAGCTGGTGGAGTCCGGAGGAGGACTGGTGCAGCCAGGAGGC |
| | | TCCCTGCGGCTGTCTTGCGCAGCCAGCGGCTTCACATTTTCTGAGTTCGG |
| | | CATGCACTGGGTGAGACAGGCCCCCGGCAAGGGACTGGAGTGGGTGTC |
| | | CTACATCAGCTCCGGCGGCTCTACCATCTACTATGCCGACAGCGTGAAG |
| | | GGCCGGTTTACAATCTCCAGAGATAACGCCAAGAACAGCCTGTACCTGC |
| | | AGATGAACAGCCTGAGGGCCGAGGACACCGCCGTGTACTATTGTGCCC |
| | | GCGACTGGGTGGATTATTGGGGCCAGGGCACCCTGGTGACAGTGTCTAG |
| | | CGCTAGCACAAAGGGCCCCTCCGTGTTTCCTCTGGCCCCATCCTCTAAG |
| | | TCCACCTCTGGAGGAACAGCCGCCCTGGGCTGTCTGGTGAAGGATTACT |
| | | TCCCTGAGCCAGTGACCGTGTCCTGGAACTCTGGGGCCCTGACCAGCGG |
| | | AGTGCACACATTTCCCGCCGTGCTGCAGAGCTCCGGACTGTACTCCCTG |
| | | TCTAGCGTGGTGACCGTGCCTTCCTCTAGCCTGGGCACCCAGACATATA |
| | | TCTGCAACGTGAATCACAAGCCTTCCAATACAAAGGTCGACAAGAAGG |
| | | TGGAGCCAAAGTCTTGTGATAAGACCCACACATGCCCACCTTGTCCGGC |
| | | GCCAGAGGCCGCCGGAGGACCAAGCGTGTTCCTGTTTCCACCCAAGCCC |
| | | AAGGACACCCTGATGATCTCCCGGACCCCAGAGGTGACATGCGTGGTG |
| | | GTGAGCGTGTCCCACGAGGACCCCGAGGTGAAGTTTAACTGGTACGTG |
| | | GATGGCGTGGAGGTGCACAATGCCAAGACAAAGCCCCGGGAGGAGCAG |
| | | TACAATTCTACCTATAGAGTGGTGAGCGTGCTGACAGTGCTGCACCAGG |
| | | ATTGGCTGAACGGCAAGGAGTATAAGTGTAAGGTGAGCAATAAGGCCC |
| | | TGCCAGCCCCCATCGAGAAGACCATCTCCAAGGCCAAGGGCCAGCCTC |
| | | GCGAACCACAGGTGTACACTCTGCCTCCATCTCGGGACGAGCTGACTAA |
| | | GAACCAGGTCAGTCTGACCTGTCTGGTGAAAGGATTCTATCCCAGCGAT |
| | | ATCGCTGTGGAGTGGGAATCCAATGGCCAGCCTGAGAACAATTACAAG |
| | | ACCACACCCCTGTGCTGGACTCTGATGGCAGTTTCTTTCTGTATAGTAA |
| | | GCTGACCGTCGATAAATCACGATGGCAGCAGGGGAACGTGTTCAGCTG |
| | | TTCAGTGATGCACGAAGCCCTGCACAACCATTACACCCAGAAGAGCCTG |
| | | AGCCTGTCTCCCGGC |
| 236 | 20895 | EVQLVESGGGLVQPGGSLRLSCAASGFTFSEFGMHWVRQAPGKGLEWVA |
| | | YISSGGSTIYYADSVKGRFTISRDNAKNSLYLQMNSLRAEDTAVYYCARD |
| | | WVDYWGQGTLLTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 237 | 20895 | GAGGTGCAGCTGGTGGAGAGCGGAGGAGGACTGGTGCAGCCAGGAGG |
| | | CTCCCTGCGGCTGTCTTGCGCAGCCAGCGGCTTCACATTTTCCGAGTTCG |
| | | GCATGCACTGGGTGAGACAGGCCCCCGGCAAGGGACTGGAGTGGGTGG |
| | | CCTACATCAGCTCCGGCGGCTCCACCATCTACTATGCCGACTCTGTGAA |
| | | GGGCCGGTTTACAATCAGCAGAGATAACGCCAAGAACAGCCTGTACCT |
| | | GCAGATGAACTCTCTGAGGGCCGAGGACACCGCCGTGTACTATTGTGCC |
| | | CGCGACTGGGTGGATTATTGGGGCCAGGGCACCCTGCTGACAGTGTCTA |
| | | GCGCTAGCACAAAGGGCCCCTCCGTGTTTCCTCTGGCCCCATCCTCTAA |
| | | GTCCACCTCTGGAGGAACAGCCGCCCTGGGCTGTCTGGTGAAGGATC |
| | | TTCCCTGAGCCAGTGACCGTGTCCTGGAACTCTGGGGCCCTGACCAGCG |
| | | GAGTGCACACATTTCCCGCCGTGCTGCAGAGCTCCGGACTGTACTCCCT |
| | | GTCTAGCGTGGTGACCGTGCCTTCCTCTAGCCTGGGCACCCAGACATAT |
| | | ATCTGCAACGTGAATCACAAGCCTTCCAATACAAAGGTCGACAAGAAG |
| | | GTGGAGCCAAAGTCTTGTGATAAGACCCACACATGCCCACCTTGTCCGG |
| | | CGCCAGAGGCCGCCGGAGGACCAAGCGTGTTCCTGTTTCCACCCAAGCC |
| | | CAAGGACACCCTGATGATCTCCCGGACCCCAGAGGTGACATGCGTGGT |
| | | GGTGAGCGTGTCCCACGAGGACCCCGAGGTGAAGTTTAACTGGTACGT |
| | | GGATGGCGTGGAGGTGCACAATGCCAAGACAAAGCCCCGGGAGGAGCA |
| | | GTACAATTCTACCTATAGAGTGGTGAGCGTGCTGACAGTGCTGCACCAG |
| | | GATTGGCTGAACGGCAAGGAGTATAAGTGTAAGGTGAGCAATAAGGCC |
| | | CTGCCAGCCCCCATCGAGAAGACCATCTCCAAGGCCAAGGGCCAGCCT |
| | | CGCGAACCACAGGTGTACACTCTGCCTCCATCTCGGGACGAGCTGACTA |
| | | AGAACCAGGTCAGTCTGACCTGTCTGGTGAAAGGATTCTATCCCAGCGA |
| | | TATCGCTGTGGAGTGGGAATCCAATGGCCAGCCTGAGAACAATTACAA |
| | | GACCACACCCCCTGTGCTGGACTCTGATGGCAGTTTCTTTCTGTATAGTA |
| | | AGCTGACCGTCGATAAATCACGATGGCAGCAGGGGAACGTGTTCAGCT |
| | | GTTCAGTGATGCACGAAGCCCTGCACAACCATTACACCCAGAAGAGCCT |
| | | GAGCCTGTCTCCCGGC |
| 238 | 20896 | EVQLVESGGGLVQPGGSLRLSCAASGFTFSEFGMHWVRQAPGKGLEWVA |
| | | YISSGGSTIYYADSVKGRFTISRDNAKNSLYLQMNSLRAEDTAMYYCARD |
| | | WVDYWGQGTLLTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 239 | 20896 | GAGGTGCAGCTGGTGGAGAGCGGAGGAGGACTGGTGCAGCCAGGAGG |
| | | CTCCCTGCGGCTGTCTTGCGCAGCCAGCGGCTTCACATTTTCCGAGTTCG |
| | | GCATGCACTGGGTGAGACAGGCCCCCGGCAAGGGACTGGAGTGGGTGG |
| | | CCTACATCAGCTCCGGCGGCTCCACCATCTACTATGCCGACTCTGTGAA |
| | | GGGCCGGTTTACAATCAGCAGAGATAACGCCAAGAACAGCCTGTACCT |
| | | GCAGATGAACTCTCTGAGGGCCGAGGACACCGCCATGTACTATTGTGCC |
| | | CGCGACTGGGTGGATTATTGGGGCCAGGGCACCCTGCTGACAGTGTCTA |
| | | GCGCTAGCACAAAGGGCCCCTCCGTGTTTCCTCTGGCCCCATCCTCTAA |
| | | GTCCACCTCTGGAGGAACAGCCGCCCTGGGCTGTCTGGTGAAGGATTAC |
| | | TTCCCTGAGCCAGTGACCGTGTCCTGGAACTCTGGGGCCCTGACCAGCG |
| | | GAGTGCACACATTTCCCGCCGTGCTGCAGAGCTCCGGACTGTACTCCCT |
| | | GTCTAGCGTGGTGACCGTGCCTTCCTCTAGCCTGGGCACCCAGACATAT |
| | | ATCTGCAACGTGAATCACAAGCCTTCCAATACAAAGGTCGACAAGAAG |
| | | GTGGAGCCAAAGTCTTGTGATAAGACCCACACATGCCCACCTTGTCCGG |
| | | CGCCAGAGGCCGCCGGAGGACCAAGCGTGTTCCTGTTTCCACCCAAGCC |
| | | CAAGGACACCCTGATGATCTCCCGGACCCCAGAGGTGACATGCGTGGT |
| | | GGTGAGCGTGTCCCACGAGGACCCCGAGGTGAAGTTTAACTGGTACGT |
| | | GGATGGCGTGGAGGTGCACAATGCCAAGACAAAGCCCCGGGAGGAGCA |
| | | GTACAATTCTACCTATAGAGTGGTGAGCGTGCTGACAGTGCTGCACCAG |
| | | GATTGGCTGAACGGCAAGGAGTATAAGTGTAAGGTGAGCAATAAGGCC |
| | | CTGCCAGCCCCCATCGAGAAGACCATCTCCAAGGCCAAGGGCCAGCCT |
| | | CGCGAACCACAGGTGTACACTCTGCCTCCATCTCGGGACGAGCTGACTA |
| | | AGAACCAGGTCAGTCTGACCTGTCTGGTGAAAGGATTCTATCCCAGCGA |
| | | TATCGCTGTGGAGTGGGAATCCAATGGCCAGCCTGAGAACAATTACAA |
| | | GACCACACCCCCTGTGCTGGACTCTGATGGCAGTTTCTTTCTGTATAGTA |
| | | AGCTGACCGTCGATAAATCACGATGGCAGCAGGGGAACGTGTTCAGCT |
| | | GTTCAGTGATGCACGAAGCCCTGCACAACCATTACACCCAGAAGAGCCT |
| | | GAGCCTGTCTCCCGGC |
| 240 | 20897 | EIVLTQSPATLSLSPGERATLSCKASENVGSYVSWYQQKPGQAPRLLIYGAS |
| | | NRYTGIPARFSGSGSGTDFTLTISSLEPEDFAVYYCGQSYSYPLTFGGGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 241 | 20897 | GAGATCGTGCTGACCCAGTCCCCAGCCACACTGTCTCTGAGCCCAGGAG |
| | | AGAGGGCCACACTGTCTTGCAAGGCCAGCGAGAACGTGGGCAGCTACG |
| | | TGTCCTGGTATCAGCAGAAGCCAGGACAGGCCCCTCGGCTGCTGATCTA |
| | | CGGGGCCAGCAATCGGTATACCGGCATCCCCGCAAGATTCTCCGGCTCT |
| | | GGCAGCGGCACAGACTTTACCCTGACAATCAGCTCCCTGGAGCCTGAGG |
| | | ATTTCGCCGTGTACTATTGTGGCCAGTCCTACTCTTATCCACTGACCTTT |
| | | GGCGGCGGCACAAAGGTGGAGATCAAGAGGACCGTGGCGGCGCCCAGT |
| | | GTCTTCATTTTTCCCCCTAGCGACGAACAGCTGAAGTCTGGGACAGCCA |
| | | GTGTGGTCTGTCTGCTGAACAACTTCTACCCTAGAGAGGCTAAAGTGCA |
| | | GTGGAAGGTCGATAACGCACTGCAGTCCGGAAATTCTCAGGAGAGTGT |
| | | GACTGAACAGGACTCAAAAGATAGCACCTATTCCCTGTCAAGCACACTG |
| | | ACTCTGAGCAAGGCCGACTACGAGAAGCATAAAGTGTATGCTTGTGAA |
| | | GTCACCCACCAGGGGCTGAGTTCACCAGTCACAAAATCATTCAACAGA |
| | | GGGGAGTGC |
| 242 | 20898 | EIVLTQSPATLSLSPGERATLSCKASENVGSYVSWYQQKPGQAPRLLIYGAS |
| | | NRYTGVPARFSGSGSGTDFTLTISSLEPEDFAVYYCGQSYSYPLTFGGGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 243 | 20898 | GAGATCGTGCTGACCCAGTCCCCAGCCACACTGTCTCTGAGCCCAGGAG |
| | | AGAGGGCCACACTGTCTTGCAAGGCCAGCGAGAACGTGGGCAGCTACG |
| | | TGTCCTGGTATCAGCAGAAGCCAGGACAGGCCCCTCGGCTGCTGATCTA |
| | | CGGGGCCAGCAATCGGTATACCGGAGTGCCCGCCAGATTCTCCGGCTCT |
| | | GGCAGCGGCACAGACTTTACCCTGACAATCAGCTCCCTGGAGCCTGAGG |
| | | ATTTCGCCGTGTACTATTGTGGCCAGTCCTACTCTTATCCACTGACCTTT |
| | | GGCGGCGGCACAAAGGTGGAGATCAAGAGGACCGTGGCGGCGCCCAGT |
| | | GTCTTCATTTTTCCCCCTAGCGACGAACAGCTGAAGTCTGGGACAGCCA |
| | | GTGTGGTCTGTCTGCTGAACAACTTCTACCCTAGAGAGGCTAAAGTGCA |
| | | GTGGAAGGTCGATAACGCACTGCAGTCCGGAAATTCTCAGGAGAGTGT |
| | | GACTGAACAGGACTCAAAAGATAGCACCTATTCCCTGTCAAGCACACTG |
| | | ACTCTGAGCAAGGCCGACTACGAGAAGCATAAAGTGTATGCTTGTGAA |
| | | GTCACCCACCAGGGGCTGAGTTCACCAGTCACAAAATCATTCAACAGA |
| | | GGGGAGTGC |
| 244 | 20899 | EIVMTQSPATLSLSPGERVTLSCKASENVGSYVSWYQQKPGQAPRLLIYGA |
| | | SNRYTGVPARFSGSGSGTDFTLTISSVEPEDFAVYYCGQSYSYPLTFGGGTK |
| | | LELKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| 245 | 20899 | GAGATCGTGATGACCCAGTCCCCAGCCACACTGTCTCTGAGCCCAGGAG |
| | | AGAGGGTGACACTGTCTTGCAAGGCCAGCGAGAACGTGGGCAGCTACG |
| | | TGTCCTGGTATCAGCAGAAGCCAGGACAGGCCCCTCGGCTGCTGATCTA |
| | | CGGGGCCAGCAATCGGTATACCGGAGTGCCCGCCAGATTCTCCGGCTCT |
| | | GGCAGCGGCACAGACTTTACCCTGACAATCAGCTCCGTGGAGCCTGAG |
| | | GATTTCGCCGTGTACTATTGTGGCCAGTCCTACTCTTATCCACTGACCTT |
| | | TGGCGGCGGCACAAAGCTGGAGCTGAAGAGGACCGTGGCGGCGCCCAG |
| | | TGTCTTCATTTTTCCCCCTAGCGACGAACAGCTGAAGTCTGGGACAGCC |
| | | AGTGTGGTCTGTCTGCTGAACAACTTCTACCCTAGAGAGGCTAAAGTGC |
| | | AGTGGAAGGTCGATAACGCACTGCAGTCCGGAAATTCTCAGGAGAGTG |
| | | TGACTGAACAGGACTCAAAAGATAGCACCTATTCCCTGTCAAGCACACT |
| | | GACTCTGAGCAAGGCCGACTACGAGAAGCATAAAGTGTATGCTTGTGA |
| | | AGTCACCCACCAGGGGCTGAGTTCACCAGTCACAAAATCATTCAACAG |
| | | AGGGGAGTGC |
| 246 | 20900 | EIVMTQSPATLSLSPGERVTLTSCKASENVGSYVSWYQQKPGQAPRLLIYGA |
| | | SNRYTGVPARFSGSGSGTDFTLTISSVEPEDLAVYYCGQSYSYPLTFGGGTK |
| | | LELKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| 247 | 20900 | GAGATCGTGATGACCCAGTCCCCAGCCACACTGTCTCTGAGCCCAGGAG |
| | | AGAGGGTGACACTGTCTTGCAAGGCCAGCGAGAACGTGGGCAGCTACG |
| | | TGTCCTGGTATCAGCAGAAGCCAGGACAGGCCCCTCGGCTGCTGATCTA |
| | | CGGGGCCAGCAATCGGTATACCGGAGTGCCCGCCAGATTCTCCGGCTCT |
| | | GGCAGCGGCACAGACTTTACCCTGACAATCAGCTCCGTGGAGCCTGAG |
| | | GATCTGGCCGTGTACTATTGTGGCCAGTCCTACTCTTATCCACTGACCTT |
| | | TGGCGGCGGCACAAAGCTGGAGCTGAAGAGGACCGTGGCGGCGCCCAG |
| | | TGTCTTCATTTTTCCCCCTAGCGACGAACAGCTGAAGTCTGGGACAGCC |
| | | AGTGTGGTCTGTCTGCTGAACAACTTCTACCCTAGAGAGGCTAAAGTGC |
| | | AGTGGAAGGTCGATAACGCACTGCAGTCCGGAAATTCTCAGGAGAGTG |
| | | TGACTGAACAGGACTCAAAAGATAGCACCTATTCCCTGTCAAGCACACT |
| | | GACTCTGAGCAAGGCCGACTACGAGAAGCATAAAGTGTATGCTTGTGA |
| | | AGTCACCCACCAGGGGCTGAGTTCACCAGTCACAAAATCATTCAACAG |
| | | AGGGGAGTGC |
| 248 | 20901 | QVQLQESGPGLVKPSETLSLTCTVSGFSLISYGVHWIRQPPGKGLEWIGVIW |
| | | SGGSTDYNPSLKSRVTISVDTSKNQFSLKLSSVTAADTAVYYCARNPLTAT |
| | | VMDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 249 | 20901 | CAGGTGCAGCTGCAGGAGTCCGGACCAGGACTGGTGAAGCCATCTGAG |
| | | ACACTGAGCCTGACCTGCACAGTGTCCGGCTTTTCTCTGATCAGCTACG |
| | | GAGTGCACTGGATCAGGCAGCCACCTGGCAAGGGACTGGAGTGGATCG |
| | | GCGTGATCTGGTCCGGCGGCTCTACCGACTATAACCCCAGCCTGAAGTC |
| | | CCGCGTGACCATCTCCGTGGATACATCTAAGAATCAGTTCAGCCTGAAG |
| | | CTGAGCTCCGTGACCGCCGCCGACACAGCCGTGTACTATTGTGCCCGGA |
| | | ACCCTCTGACCGCCACAGTGATGGATTACTGGGGCCAGGGCACCCTGGT |
| | | GACAGTGTCTAGCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAACCACAGGTGTACACTCTGCCTCCATCTCGGGA |
| | | CGAGCTGACTAAGAACCAGGTCAGTCTGACCTGTCTGGTGAAAGGATTC |
| | | TATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 250 | 20902 | QVQLQESGPGLVKPSETLSLTCTVSGFSLISYGVHWVRQPPGKGLEWLGVI |
| | | WSGGSTDYNPSLKSRVTISKDTSKNQFSLKLSSVTAADTAVYYCARNPLTA |
| | | TVMDYWGQGTLVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 251 | 20902 | CAGGTGCAGCTGCAGGAGTCCGGACCAGGACTGGTGAAGCCATCTGAG |
| | | ACACTGAGCCTGACCTGCACAGTGTCCGGCTTTTCTCTGATCAGCTACG |
| | | GAGTGCACTGGGTGAGGCAGCCACCTGGCAAGGGACTGGAGTGGCTGG |
| | | GCGTGATCTGGTCCGGAGGCTCTACCGACTATAACCCCAGCCTGAAGTC |
| | | CCGCGTGACCATCTCCAAGGATACATCTAAGAATCAGTTCAGCCTGAAG |
| | | CTGAGCTCCGTGACCGCCGCCGACACAGCCGTGTACTATTGTGCCCGGA |
| | | ACCCTCTGACCGCCACAGTGATGGATTACTGGGGCCAGGGCACCCTGGT |
| | | GACAGTGTCTAGCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAACCACAGGTGTACACTCTGCCTCCATCTCGGGA |
| | | CGAGCTGACTAAGAACCAGGTCAGTCTGACCTGTCTGGTGAAAGGATTC |
| | | TATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 252 | 20903 | QVQLQESGPGLVKPSETLSITCTVSGFSLISYGVHWVRQPPGKGLEWLGVI |
| | | WSGGSTDYNPSLKSRVTISKDTSKNQVSFKLSSLTAADTAIYYCARNPLTAT |
| | | VMDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 253 | 20903 | CAGGTGCAGCTGCAGGAGTCCGGACCAGGACTGGTGAAGCCATCTGAG |
| | | ACACTGAGCATCACCTGCACAGTGTCCGGCTTTTCTCTGATCAGCTACG |
| | | GAGTGCACTGGGTGAGGCAGCCACCTGGCAAGGGACTGGAGTGGCTGG |
| | | GCGTGATCTGGTCCGGAGGCTCTACCGACTATAACCCCAGCCTGAAGTC |
| | | CCGCGTGACCATCTCCAAGGATACATCTAAGAATCAGGTGAGCTTCAAG |
| | | CTGAGCTCCCTGACCGCCGCCGACACAGCCATCTACTATTGTGCCCGGA |
| | | ACCCTCTGACCGCCACAGTGATGGATTACTGGGGCCAGGGCACCCTGGT |
| | | GACAGTGTCTAGCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAACCACAGGTGTACACTCTGCCTCCATCTCGGGA |
| | | CGAGCTGACTAAGAACCAGGTCAGTCTGACCTGTCTGGTGAAAGGATTC |
| | | TATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 254 | 20904 | QVQLQESGPGLVKPSETLSITCTVSGFSLISYGVHWVRQPPGKGLEWLGVI |
| | | WSGGSTDYNPSLKSRVTISKDNSKNQVSFKLSSLTAADTAIYYCARNPLTA |
| | | TVMDYWGQGTLVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 255 | 20904 | CAGGTGCAGCTGCAGGAGTCCGGACCAGGACTGGTGAAGCCATCTGAG |
| | | ACACTGAGCATCACCTGCACAGTGTCCGGCTTTTCTCTGATCAGCTACG |
| | | GAGTGCACTGGGTGAGGCAGCCACCTGGCAAGGGACTGGAGTGGCTGG |
| | | GCGTGATCTGGTCCGGAGGCTCTACCGACTATAACCCCAGCCTGAAGTC |
| | | CCGCGTGACAATCTCCAAGGATAACTCTAAGAATCAGGTGAGCTTCAAG |
| | | CTGAGCTCCCTGACCGCCGCCGACACAGCCATCTACTATTGTGCCCGGA |
| | | ATCCTCTGACCGCCACAGTGATGGATTACTGGGGCCAGGGCACCCTGGT |
| | | GACAGTGTCTAGCGCTAGCACAAAGGGCCCCTCCGTGTTTCCTCTGGCC |
| | | CCATCCTCTAAGTCCACCTCTGGAGGAACAGCCGCCCTGGGCTGTCTGG |
| | | TGAAGGATTACTTCCCTGAGCCAGTGACCGTGTCCTGGAACTCTGGGGC |
| | | CCTGACCAGCGGAGTGCACACATTTCCCGCCGTGCTGCAGAGCTCCGGA |
| | | CTGTACTCCCTGTCTAGCGTGGTGACCGTGCCTTCCTCTAGCCTGGGCAC |
| | | CCAGACATATATCTGCAACGTGAATCACAAGCCTTCCAATACAAAGGTC |
| | | GACAAGAAGGTGGAGCCAAAGTCTTGTGATAAGACCCACACATGCCCA |
| | | CCTTGTCCGGCGCCAGAGGCCGCCGGAGGACCAAGCGTGTTCCTGTTTC |
| | | CACCCAAGCCCAAGGACACCCTGATGATCTCCCGGACCCCAGAGGTGA |
| | | CATGCGTGGTGGTGAGCGTGTCCCACGAGGACCCCGAGGTGAAGTTTA |
| | | ACTGGTACGTGGATGGCGTGGAGGTGCACAATGCCAAGACAAAGCCCC |
| | | GGGAGGAGCAGTACAATTCTACCTATAGAGTGGTGAGCGTGCTGACAG |
| | | TGCTGCACCAGGATTGGCTGAACGGCAAGGAGTATAAGTGTAAGGTGA |
| | | GCAATAAGGCCCTGCCAGCCCCCATCGAGAAGACCATCTCCAAGGCCA |
| | | AGGGCCAGCCTCGCGAACCACAGGTGTACACTCTGCCTCCATCTCGGGA |
| | | CGAGCTGACTAAGAACCAGGTCAGTCTGACCTGTCTGGTGAAAGGATTC |
| | | TATCCCAGCGATATCGCTGTGGAGTGGGAATCCAATGGCCAGCCTGAGA |
| | | ACAATTACAAGACCACACCCCCTGTGCTGGACTCTGATGGCAGTTTCTT |
| | | TCTGTATAGTAAGCTGACCGTCGATAAATCACGATGGCAGCAGGGGAA |
| | | CGTGTTCAGCTGTTCAGTGATGCACGAAGCCCTGCACAACCATTACACC |
| | | CAGAAGAGCCTGAGCCTGTCTCCCGGC |
| 256 | 20905 | DIQMTQSPSSLSASVGDRVTITCRASQNVGTNVAWFQQKPGKAPKSLIYSA |
| | | SYRDSGVPSRFSGSGSGTDFTLTISSLQPEDFATYYCQQYNSYPLTFGGGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| 257 | 20905 | GACATCCAGATGACACAGTCCCCAAGCTCCCTGTCCGCCTCTGTGGGCG |
| | | ATAGGGTGACCATCACATGCAGAGCCTCTCAGAACGTGGGCACCAATG |
| | | TGGCCTGGTTTCAGCAGAAGCCCGGCAAGGCCCCTAAGTCCCTGATCTA |
| | | CAGCGCCTCCTATAGGGACTCTGGAGTGCCTAGCCGCTTCTCTGGCAGC |
| | | GGCTCCGGAACCGACTTTACCCTGACAATCTCTAGCCTGCAGCCTGAGG |
| | | ATTTCGCCACATACTATTGTCAGCAGTACAACAGCTATCCACTGACCTTT |
| | | GGCGGCGGCACAAAGGTGGAGATCAAGCGGACAGTGGCGGCGCCCAGT |
| | | GTCTTCATTTTTCCCCCTAGCGACGAACAGCTGAAGTCTGGGACAGCCA |
| | | GTGTGGTCTGTCTGCTGAACAACTTCTACCCTAGAGAGGCTAAAGTGCA |
| | | GTGGAAGGTCGATAACGCACTGCAGTCCGGAAATTCTCAGGAGAGTGT |
| | | GACTGAACAGGACTCAAAAGATAGCACCTATTCCCTGTCAAGCACACTG |
| | | ACTCTGAGCAAGGCCGACTACGAGAAGCATAAAGTGTATGCTTGTGAA |
| | | GTCACCCACCAGGGGCTGAGTTCACCAGTCACAAAATCATTCAACAGA |
| | | GGGGAGTGC |
| 258 | 20906 | DIQMTQSPSSLSASVGDRVTITCRASQNVGTNVAWYQQKPGKAPKALIYSA |
| | | SYRDSGVPSRFSGSGSGTDFTLTISSLQPEDFATYYCQQYNSYPLTFGGGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| 259 | 20906 | GACATCCAGATGACACAGTCCCCAAGCTCCCTGTCCGCCTCTGTGGGCG |
| | | ATAGGGTGACCATCACATGCAGAGCCTCTCAGAACGTGGGCACCAATG |
| | | TGGCCTGGTACCAGCAGAAGCCCGGCAAGGCCCCTAAGGCCCTGATCT |
| | | ACAGCGCCTCCTATAGGGACTCTGGAGTGCCTAGCCGCTTCTCTGGCAG |
| | | CGGCTCCGGAACCGACTTTACCCTGACAATCTCTAGCCTGCAGCCTGAG |
| | | GATTTCGCCACATACTATTGTCAGCAGTACAACTCCTATCCACTGACCTT |
| | | TGGCGGCGGCACAAAGGTGGAGATCAAGCGGACAGTGGCGGCGCCCAG |
| | | TGTCTTCATTTTTCCCCCTAGCGACGAACAGCTGAAGTCTGGGACAGCC |
| | | AGTGTGGTCTGTCTGCTGAACAACTTCTACCCTAGAGAGGCTAAAGTGC |
| | | AGTGGAAGGTCGATAACGCACTGCAGTCCGGAAATTCTCAGGAGAGTG |
| | | TGACTGAACAGGACTCAAAAGATAGCACCTATTCCCTGTCAAGCACACT |
| | | GACTCTGAGCAAGGCCGACTACGAGAAGCATAAAGTGTATGCTTGTGA |
| | | AGTCACCCACCAGGGGCTGAGTTCACCAGTCACAAAATCATTCAACAG |
| | | AGGGGAGTGC |
| 260 | 20907 | DIQMTQSPSSLSASVGDRVTVTCRASQNVGTNVAWYQQKPGKAPKALIYS |
| | | ASYRDSGVPSRFSGSGSGTDFTLTISSVQPEDFATYYCQQYNSYPLTFGGGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |
| 261 | 20907 | GACATCCAGATGACACAGTCCCCAAGCTCCCTGTCCGCCTCTGTGGGCG |
| | | ATAGGGTGACCGTGACATGCAGAGCCTCTCAGAACGTGGGCACCAATG |
| | | TGGCCTGGTACCAGCAGAAGCCCGGCAAGGCCCCTAAGGCCCTGATCT |
| | | ACAGCGCCTCCTATAGGGACTCTGGAGTGCCTAGCCGCTTCTCTGGCAG |
| | | CGGCTCCGGAACCGACTTTACCCTGACAATCTCTAGCGTGCAGCCTGAG |
| | | GATTTCGCCACATACTATTGTCAGCAGTACAACTCCTATCCACTGACCTT |
| | | TGGCGGCGGCACAAAGCTGGAGATCAAGCGGACAGTGGCGGCGCCCAG |
| | | TGTCTTCATTTTTCCCCCTAGCGACGAACAGCTGAAGTCTGGGACAGCC |
| | | AGTGTGGTCTGTCTGCTGAACAACTTCTACCCTAGAGAGGCTAAAGTGC |
| | | AGTGGAAGGTCGATAACGCACTGCAGTCCGGAAATTCTCAGGAGAGTG |
| | | TGACTGAACAGGACTCAAAAGATAGCACCTATTCCCTGTCAAGCACACT |
| | | GACTCTGAGCAAGGCCGACTACGAGAAGCATAAAGTGTATGCTTGTGA |
| | | AGTCACCCACCAGGGGCTGAGTTCACCAGTCACAAAATCATTCAACAG |
| | | AGGGGAGTGC |
| 262 | 20908 | DIQMTQSPSSLSASVGDRVTVTCRASQNVGTNVAWYQQKPGKAPKALIYS |
| | | ASYRDSGVPSRFSGSGSGTDFTLTISSVQPEDLATYYCQQYNSYPLTFGGGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |
| 263 | 20908 | GACATCCAGATGACACAGTCCCCAAGCTCCCTGTCCGCCTCTGTGGGCG |
| | | ATAGGGTGACCGTGACATGCAGAGCCTCTCAGAACGTGGGCACCAATG |
| | | TGGCCTGGTACCAGCAGAAGCCCGGCAAGGCCCCTAAGGCCCTGATCT |
| | | ACAGCGCCTCCTATAGGGACTCTGGAGTGCCTAGCCGCTTCTCTGGCAG |
| | | CGGCTCCGGAACCGACTTTACCCTGACAATCTCTAGCGTGCAGCCTGAG |
| | | GATCTGGCCACATACTATTGTCAGCAGTACAACTCCTATCCACTGACCT |
| | | TTGGCGGCGGCACAAAGCTGGAGATCAAGCGGACAGTGGCGGCGCCCA |
| | | GTGTCTTCATTTTTCCCCCTAGCGACGAACAGCTGAAGTCTGGGACAGC |
| | | CAGTGTGGTCTGTCTGCTGAACAACTTCTACCCTAGAGAGGCTAAAGTG |
| | | CAGTGGAAGGTCGATAACGCACTGCAGTCCGGAAATTCTCAGGAGAGT |
| | | GTGACTGAACAGGACTCAAAAGATAGCACCTATTCCCTGTCAAGCACAC |
| | | TGACTCTGAGCAAGGCCGACTACGAGAAGCATAAAGTGTATGCTTGTG |
| | | AAGTCACCCACCAGGGGCTGAGTTCACCAGTCACAAAATCATTCAACA |
| | | GAGGGGAGTGC |
| 264 | 16833 | EVQLVQSGAEVKKPGESLRISCKGSGYSFSTYWISWVRQMPGKGLEWMGK |
| | | IYPGDSYTNYSPSFQGQVTISADKSISTAYLQSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGQVQLVQSG |
| | | AEVKKPGASVKVSCKASGYSFTGYTMNWVRQAPGQGLEWMGLITPYNGA |
| | | SSYNQKFRGKATMTVDTSTSTVYMELSSLRSEDTAVYYCARGGYDGRGFD |
| | | YWGQGTLVTVSSGGGGSGGGGSGGGGSDIQMTQSPSSLSASVGDRVTITCS |
| | | ASSSYSYMHWYQQKSGKAPKLLIYDTSKLASGVPSRFSGSGSGTDFTLTISS |
| | | LQPEDFATYYCQQWSKHPLTFGQGTKLEIK |
| 265 | 16833 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCACAGGTGTACGTGCTGCCCCCTTCCAGGGACGAG |
| | | CTGACCAAGAACCAGGTGTCTCTGCTGTGCCTGGTGAAGGGCTTCTATC |
| | | CCTCTGATATCGCCGTGGAGTGGGAGAGCAATGGCCAGCCTGAGAACA |
| | | ATTACCTGACATGGCCACCCGTGCTGGACTCCGATGGCTCTTTCTTTCTG |
| | | TATAGCAAGCTGACCGTGGACAAGTCCCGCTGGCAGCAGGGCAACGTG |
| | | TTTTCCTGCTCTGTGATGCACGAGGCCCTGCACAATCACTACACCCAGA |
| | | AGAGCCTGAGCTTAAGCCCAGGAGGAGGAGGAGGACAGGTGCAGCTGG |
| | | TGCAGTCCGGAGCCGAGGTGAAGAAGCCTGGGGCCAGCGTGAAGGTGA |
| | | GCTGTAAGGCCAGCGGCTACTCCTTCACAGGCTATACCATGAACTGGGT |
| | | GCGGCAGGCCCCTGGACAGGGACTGGAGTGGATGGGCCTGATCACACC |
| | | ATACAACGGGGCCAGCTCCTATAATCAGAAGTTTAGGGGCAAGGCCAC |
| | | CATGACAGTGGACACCAGCACATCCACCGTGTATATGGAGCTGTCTAGC |
| | | CTGCGCAGCGAGGATACCGCCGTGTACTATTGCGCCAGAGGCGGATAC |
| | | GACGGCAGAGGCTTCGATTATTGGGGCCAGGGCACACTGGTGACCGTG |
| | | TCCTCTGGAGGAGGAGGCTCCGGCGGAGGAGGCTCTGGCGGCGGCGGC |
| | | AGCGACATCCAGATGACACAGTCCCCTAGCTCCCTGTCTGCCAGCGTGG |
| | | GCGATAGGGTGACAATCACCTGTTCCGCCTCTAGCTCCGTGTCTTACAT |
| | | GCACTGGTATCAGCAGAAGAGCGGCAAGGCCCCAAAGCTGCTGATCTA |
| | | CGATACCTCCAAGCTGGCCTCTGGCGTGCCCAGCAGATTCTCCGGCTCT |
| | | GGCAGCGGCACAGACTTTACACTGACCATCTCTAGCCTGCAGCCTGAGG |
| | | ATTTCGCCACCTACTATTGTCAGCAGTGGAGCAAGCACCCACTGACATT |
| | | TGGCCAGGGCACCAAGCTGGAGATCAAG |
| 266 | 16839 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGQVQLVQSG |
| | | AEVVKPGASVKMSCKASGYTFTGYNIHWVKQAPGQGLEWIGAIYPGNGD |
| | | TSYKQKFRGRATLTADTSTSTVYMELSSLRSEDSAVYYCARGETARATFAY |
| | | WGQGTLVTVSSGGGGSGGGGSGGGGSDIQMTQSPSSLSASVGDRVTITCSA |
| | | SQDIGNFLNWYQQKPGKTVKVLIYYTSSLYSGVPSRFSGSGSGTDYTLTISS |
| | | LQPEDFATYYCQQYSKLPLTFGQGTKLELK |
| 267 | 16839 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCACAGGTGTACGTGCTGCCCCCTTCCAGGGACGAG |
| | | CTGACCAAGAACCAGGTGTCTCTGCTGTGCCTGGTGAAGGGCTTCTATC |
| | | CCTCTGATATCGCCGTGGAGTGGGAGAGCAATGGCCAGCCTGAGAACA |
| | | ATTACCTGACATGGCCACCCGTGCTGGACAGCGATGGCTCCTTCTTTCT |
| | | GTATAGCAAGCTGACCGTGGACAAGTCCCGCTGGCAGCAGGGCAACGT |
| | | GTTTTCCTGCTCTGTGATGCACGAGGCCCTGCACAATCACTACACACAG |
| | | AAGAGCCTGAGCTTAAGCCCTGGAGGAGGAGGAGGACAGGTGCAGCTG |
| | | GTGCAGAGCGGAGCCGAGGTGGTGAAGCCAGGGGCCAGCGTGAAGATG |
| | | TCTTGTAAGGCCAGCGGCTACACCTTCACAGGCTATAACATCCACTGGG |
| | | TGAAGCAGGCCCCTGGACAGGGACTGGAGTGGATCGGAGCCATCTACC |
| | | CAGGCAATGGCGACACCTCCTATAAGCAGAAGTTTCGGGGCAGAGCCA |
| | | CCCTGACAGCCGATACCAGCACATCCACCGTGTACATGGAGCTGAGCTC |
| | | CCTGCGGTCTGAGGACAGCGCCGTGTACTATTGCGCCAGGGGCGAGAC |
| | | AGCAAGGGCCACCTTCGCCTATTGGGGACAGGGCACACTGGTGACCGT |
| | | GTCTAGCGGAGGAGGAGGCAGCGGAGGAGGAGGCTCCGGCGGCGGCG |
| | | GCTCTGATATCCAGATGACCCAGAGCCCTTCCTCTCTGTCTGCCAGCGT |
| | | GGGCGACAGGGTGACAATCACCTGTTCCGCCTCTCAGGATATCGGCAAC |
| | | TTCCTGAATTGGTACCAGCAGAAGCCCGGCAAGACAGTGAAGGTGCTG |
| | | ATCTACTATACCAGCTCCCTGTACTCCGGAGTGCCTAGCCGGTTCAGCG |
| | | GCTCCGGCTCTGGAACAGACTATACACTGACCATCTCTAGCCTGCAGCC |
| | | AGAGGATTTCGCCACCTACTATTGTCAGCAGTATTCCAAGCTGCCCCTG |
| | | ACATTTGGCCAGGGCACCAAGCTGGAGCTGAAG |
| 268 | 16840 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGDIQMTQSPS |
| | | SLSASVGDRVTITCSASQDIGNFLNWYQQKPGKTVKVLIYYTSSLYSGVPSR |
| | | FSGSGSGTDYTLTISSLQPEDFATYYCQQYSKLPLTFGQGTKLELKGGGGSG |
| | | GGGSGGGGSQVQLVQSGAEVVKPGASVKMSCKASGYTFTGYNIHWVKQA |
| | | PGQGLEWIGAIYPGNGDTSYKQKFRGRATLTADTSTSTVYMELSSLRSEDS |
| | | AVYYCARGETARATFAYWGQGTLVTVSS |
| 269 | 16840 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCACAGGTGTACGTGCTGCCCCCTTCCAGGGACGAG |
| | | CTGACCAAGAACCAGGTGTCTCTGCTGTGCCTGGTGAAGGGCTTCTATC |
| | | CCTCTGATATCGCCGTGGAGTGGGAGAGCAATGGCCAGCCTGAGAACA |
| | | ATTACCTGACATGGCCACCCGTGCTGGACAGCGATGGCTCCTTCTTTCT |
| | | GTATAGCAAGCTGACCGTGGACAAGTCCCGCTGGCAGCAGGGCAACGT |
| | | GTTTTCCTGCTCTGTGATGCACGAGGCCCTGCACAATCACTACACACAG |
| | | AAGAGCCTGAGCTTAAGCCCAGGAGGAGGAGGAGGCGATATCCAGATG |
| | | ACCCAGAGCCCAAGCTCCCTGAGCGCCTCCGTGGGCGACCGGGTGACC |
| | | ATCACATGTTCTGCCAGCCAGGATATCGGCAACTTCCTGAATTGGTATC |
| | | AGCAGAAGCCCGGCAAGACAGTGAAGGTGCTGATCTACTATACCTCTA |
| | | GCCTGTACTCCGGCGTGCCTTCTAGATTTTCCGGCTCTGGCAGCGGCAC |
| | | AGACTATACCCTGACAATCTCCTCTCTGCAGCCTGAGGATTTCGCCACC |
| | | TACTATTGCCAGCAGTACAGCAAGCTGCCACTGACATTTGGCCAGGGCA |
| | | CCAAGCTGGAGCTGAAGGGAGGAGGAGGCAGCGGCGGCGGAGGCTCC |
| | | GGCGGCGGCGGCTCTCAGGTGCAGCTGGTGCAGTCCGGAGCCGAGGTG |
| | | GTGAAGCCTGGGGCCAGCGTGAAGATGTCCTGTAAGGCCTCTGGCTACA |
| | | CCTTCACAGGCTATAACATCCACTGGGTGAAGCAGGCCCCTGGACAGG |
| | | GACTGGAGTGGATCGGAGCCATCTACCCAGGCAATGGCGACACCTCCT |
| | | ATAAGCAGAAGTTTCGGGGCAGAGCCACCCTGACAGCCGATACCTCCA |
| | | CATCTACCGTGTACATGGAGCTGAGCTCCCTGCGGTCTGAGGACAGCGC |
| | | CGTGTACTATTGTGCCAGGGGCGAGACAGCAAGGGCCACCTTCGCCTAT |
| | | TGGGGACAGGGCACACTGGTGACCGTGTCTAGC |
| 270 | 16841 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGEVQLVESG |
| | | GGLVQPGGSLRLSCAASGFSFSDFAMSWVRQAPGKGLEWVATIGRVAFHT |
| | | YYPDSMKGRFTISRDNSKNTLYLQMNSLRAEDTAVYYCARHRGFDVGHFD |
| | | FWGQGTLVTVSSGGGGSGGGGSGGGGSDIQMTQSPSSLSASVGDRVTITCR |
| | | SSETLVHSSGNTYLEWYQQKPGKAPKLLIYRVSNRFSGVPSRFSGSGSGTDF |
| | | TLTISSLQPEDFATYYCFQGSFNPLTFGQGTKVEIK |
| 271 | 16841 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCTCAGGTGTACGTGCTGCCACCTAGCCGGGACGAG |
| | | CTGACCAAGAACCAGGTGTCCCTGCTGTGCCTGGTGAAGGGCTTCTATC |
| | | CCTCCGATATCGCCGTGGAGTGGGAGTCTAATGGCCAGCCTGAGAACA |
| | | ATTACCTGACCTGGCCACCCGTGCTGGACTCCGATGGCTCTTTCTTTCTG |
| | | TATTCTAAGCTGACAGTGGACAAGAGCAGGTGGCAGCAGGGCAACGTG |
| | | TTTTCCTGCTCTGTGATGCACGAGGCCCTGCACAATCACTACACCCAGA |
| | | AGAGCCTGAGCTTAAGCCCTGGAGGAGGAGGAGGAGAGGTGCAGCTGG |
| | | TGGAGAGCGGCGGCGGCCTGGTGCAGCCAGGCGGCAGCCTGCGCCTGT |
| | | CCTGTGCCGCCTCTGGCTTCAGCTTTTCCGACTTCGCCATGAGCTGGGTG |
| | | CGGCAGGCCCCCGGCAAGGGACTGGAGTGGGTGGCCACCATCGGCAGA |
| | | GTGGCCTTCCACACATACTATCCTGACTCTATGAAGGGCCGGTTTACCA |
| | | TCAGCAGAGATAACTCCAAGAATACACTGTATCTGCAGATGAACAGCCT |
| | | GCGGGCCGAGGATACCGCCGTGTACTATTGCGCAAGGCACAGAGGCTT |
| | | TGACGTGGGACACTTCGATTTTTGGGGCCAGGGCACCCTGGTGACAGTG |
| | | AGCTCCGGCGGCGGCGGCTCTGGAGGAGGAGGCAGCGGCGGAGGAGG |
| | | CTCCGACATCCAGATGACACAGTCCCCATCTAGCCTGTCTGCCAGCGTG |
| | | GGCGATAGGGTGACCATCACATGTCGCTCCTCTGAGACCCTGGTGCACA |
| | | GCTCCGGCAACACATACCTGGAGTGGTATCAGCAGAAGCCTGGCAAGG |
| | | CCCCAAAGCTGCTGATCTACAGGGTGTCTAATCGCTTCAGCGGCGTGCC |
| | | CTCCAGATTTTCCGGCTCTGGCAGCGGAACCGACTTCACCCTGACAATC |
| | | TCTAGCCTGCAGCCAGAGGATTTTGCCACATACTATTGTTTCCAGGGCA |
| | | GCTTTAATCCCCTGACCTTCGGCCAGGGCACAAAGGTGGAGATCAAG |
| 272 | 16843 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGQVQLVQSG |
| | | AEVVKPGASVKISCKASGYTFTGYFMNWVKQSPGQSLEWIGRIHPYDGDT |
| | | FYNQKFQGKATLTVDKSSNTAHMELLSLTSEDFAVYYCTRYDGSRAMDY |
| | | WGQGTTVTVSSGGGGSGGGGSGGGGSDIVLTQSPLSLAVSLGQPAIISCKA |
| | | SQSVSFAGTSLMHWYHQKPGQQPRLLIYRASNLEAGVPDRFSGSGSKTDFT |
| | | LTISPVEAEDAATYYCQQSREYPYTFGGGTKLEIK |
| 273 | 16843 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCCCAGGTGTACGTGCTGCCCCCTTCTAGGGACGAG |
| | | CTGACCAAGAACCAGGTGAGCCTGCTGTGCCTGGTGAAGGGCTTCTATC |
| | | CTTCCGATATCGCCGTGGAGTGGGAGTCTAATGGCCAGCCAGAGAACA |
| | | ATTACCTGACCTGGCCACCCGTGCTGGACAGCGATGGCTCCTTCTTTCTG |
| | | TATTCTAAGCTGACAGTGGACAAGAGCCGCTGGCAGCAGGGCAACGTG |
| | | TTTTCCTGCTCTGTGATGCACGAGGCCCTGCACAATCACTACACCCAGA |
| | | AGAGCCTGAGCTTAAGCCCAGGAGGAGGAGGAGGACAGGTGCAGGGG |
| | | TGCAGTCCGGAGCCGAGGTGGTGAAGCCTGGGGCCAGCGTGAAGATCT |
| | | CCTGTAAGGCCTCTGGCTACACCTTCACAGGCTACTTCATGAACTGGGT |
| | | GAAGCAGAGCCCAGGCCAGTCCCTGGAGTGGATCGGCAGAATCCACCC |
| | | CTACGACGGCGATACATTCTATAACCAGAAGTTTCAGGGCAAGGCCACC |
| | | CTGACCGTGGACAAAAGCTCCAATACCGCCCACATGGAGCTGCTGTCTC |
| | | TGACAAGCGAGGATTTCGCCGTGTACTATTGCACCCGGTACGACGGCAG |
| | | CAGAGCCATGGATTATTGGGGCCAGGGCACCACAGTGACAGTGTCTAG |
| | | CGGCGGCGGCGGCTCTGGAGGAGGAGGCAGCGGCGGAGGAGGCTCCG |
| | | ACATCGTGCTGACCCAGTCCCCACTGTCTCTGGCCGTGAGCCTGGGCCA |
| | | GCCTGCCATCATCTCCTGTAAGGCCAGCCAGAGCGTGAGCTTCGCCGGG |
| | | ACCAGCCTGATGCACTGGTACCACCAGAAGCCTGGCCAGCAGCCAAGG |
| | | CTGCTGATCTATAGGGCCAGCAATCTGGAGGCCGGAGTGCCAGACCGG |
| | | TTCAGCGGCTCCGGCTCTAAGACCGACTTCACCCTGACAATCTCCCCTG |
| | | TGGAGGCAGAGGATGCAGCAACATACTATTGTCAGCAGTCTAGAGAGT |
| | | ACCCATATACCTTTGGCGGCGGCACAAAGCTGGAGATCAAG |
| 274 | 8653 | QSVLTQPPSASGTPGQRVTISCSGSSSNIGTNYVYWYQQFPGTAPKLLIYRS |
| | | YQRPSGVPDRFSGSKSGSSASLAISGLQSEDEADYYCATWDDSLDGWVFG |
| | | GGTKLTVLRQPKAAPSVTLFPPSSEELQANKATLVCLISDFYPGAVTVAWK |
| | | ADSSPVKAGVETTTPSKQSNNKYAASSYLSLTPEQWKSHRSYSCQVTHEGS |
| | | TVEKTVAPTECS |
| 275 | 8653 | CAGAGCGTCCTGACTCAGCCTCCCTCCGCCTCCGGAACACCTGGGCAGA |
| | | GAGTGACTATCTCCTGTAGCGGATCAAGCTCAAACATTGGAACCAACTA |
| | | CGTGTATTGGTACCAGCAGTTCCCCGGCACAGCTCCTAAGCTGCTGATC |
| | | TATCGGAGCTACCAGAGACCAAGCGGGGTCCCCGACAGGTTTTCTGGCA |
| | | GTAAATCAGGGAGCTCCGCCAGCCTGGCTATTTCCGGCCTGCAGTCTGA |
| | | GGACGAAGCAGATTACTATTGCGCCACCTGGGACGATTCCCTGGATGGA |
| | | TGGGTCTTCGGCGGCGGCACAAAACTGACCGTCCTGAGGCAGCCAAAG |
| | | GCGGCGCCCAGTGTCACACTGTTTCCCCCTAGCTCCGAGGAACTGCAGG |
| | | CTAACAAAGCAACACTGGTGTGTCTGATCAGCGACTTCTACCCTGGAGC |
| | | TGTGACTGTCGCCTGGAAGGCTGATTCTAGTCCAGTGAAAGCAGGCGTC |
| | | GAGACCACAACTCCCTCTAAGCAGAGTAACAACAAGTACGCAGCCTCA |
| | | AGCTATCTGTCACTGACCCCAGAACAGTGGAAGAGCCACCGGAGCTATT |
| | | CCTGCCAGGTCACTCACGAAGGCTCCACTGTCGAGAAAACCGTCGCTCC |
| | | CACCGAATGTTCA |
| 276 | 14413 | DIVMTQAAFSNPVTLGTSASISCRSNKSLLHSDGITYLQRPGQSPQLLI |
| | | YRMSNLASGVPDRFSGSGSGTDFTLRISRVEAEDVGVYYCAQMVEFPRTFG |
| | | GGTKLEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| 277 | 14413 | GACATCGTGATGACACAGGCCGCCTTTAGCAACCCCGTGACCCTGGGCA |
| | | CAAGCGCCTCCATCTCTTGCCGGAGCAATAAGTCCCTGCTGCACTCCGA |
| | | TGGCATCACCTACCTGTTCTGGTATCTGCAGAGGCCCGGCCAGTCTCCT |
| | | CAGCTGCTGATCTACCGCATGTCTAACCTGGCCAGCGGAGTGCCTGACC |
| | | GGTTCAGCGGCTCCGGCTCTGGAACCGACTTCACCCTGCGGATCTCCAG |
| | | AGTGGAGGCCGAGGACGTGGGCGTGTACTATTGTGCCCAGATGGTGGA |
| | | GTTCCCAAGAACCTTTGGCGGCGGCACAAAGCTGGAGATCAAGAGGAC |
| | | AGTGGCGGCGCCCAGTGTCTTCATTTTTCCCCCTAGCGACGAACAGCTG |
| | | AAGTCTGGGACAGCCAGTGTGGTCTGTCTGCTGAACAACTTCTACCCTA |
| | | GAGAGGCTAAAGTGCAGTGGAAGGTCGATAACGCACTGCAGTCCGGAA |
| | | ATTCTCAGGAGAGTGTGACTGAACAGGACTCAAAAGATAGCACCTATTC |
| | | CCTGTCAAGCACACTGACTCTGAGCAAGGCCGACTACGAGAAGCATAA |
| | | AGTGTATGCTTGTGAAGTCACCCACCAGGGGCTGAGTTCACCAGTCACA |
| | | AAATCATTCAACAGAGGGGAGTGC |
| 278 | 8656 | QVQLVQSGAEVKKPGASVRVSCRASGYIFTESGITWVRQAPGQGLEWMG |
| | | WISGYSGDTKYAQKLQGRVTMTKDTSTTTAYMELRSLRYDDTAVYYCAR |
| | | DVQYSGSYLGAYYFDYWSPGTLVTVSSASTKGPSVFPLAPSSKSTSGGTAA |
| | | LGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPRE |
| | | PQVYVYPPSRDELTKNQVSLTCLVKGFYPSDIAVEWESNGQPENNYKTTPP |
| | | VLDSDGSFALVSKLTVDKSRWQQGNVFSCSVMHEALHNHYTQKSLSLSPG |
| 279 | 8656 | CAGGTGCAGCTGGTCCAGTCCGGGGCTGAAGTGAAAAAACCTGGGGCA |
| | | TCCGTGCGGGTGTCATGTCGGGCAAGCGGGTATATCTTTACTGAGTCTG |
| | | GAATCACCTGGGTGAGGCAGGCTCCCGGACAGGGACTGGAATGGATGG |
| | | GATGGATTTCTGGATACAGTGGCGACACAAAGTATGCACAGAAACTGC |
| | | AGGGCCGCGTCACCATGACAAAGGATACTTCAACCACAACTGCCTACAT |
| | | GGAGCTGCGGAGCCTGAGATATGACGATACAGCCGTGTACTATTGCGCC |
| | | CGGGACGTGCAGTACAGCGGGTCCTACCTGGGGGCATACTACTTCGATT |
| | | ACTGGTCACCTGGAACTCTGGTCACCGTCTCTTCAGCTAGCACCAAGGG |
| | | CCCTTCTGTGTTTCCACTGGCACCCTCAAGCAAAAGCACCTCCGGAGGA |
| | | ACAGCAGCACTGGGATGTCTGGTCAAGGACTATTTCCCCGAGCCTGTGA |
| | | CCGTCTCATGGAATAGCGGCGCACTGACTAGTGGGGTGCACACCTTTCC |
| | | CGCCGTCCTGCAGTCCTCTGGGCTGTACAGCCTGAGTTCAGTGGTCACA |
| | | GTGCCAAGCTCCTCTCTGGGAACTCAGACCTATATCTGCAACGTCAATC |
| | | ATAAACCCAGCAACACAAAGGTCGACAAGAAAGTGGAGCCCAAGAGCT |
| | | GTGATAAAACTCATACCTGCCCACCTTGTCCGGCGCCAGAACTGCTGGG |
| | | AGGACCAAGCGTGTTCCTGTTTCCACCCAAGCCTAAAGACACCCTGATG |
| | | ATTTCCCGGACTCCTGAGGTCACCTGCGTGGTCGTGGACGTGTCTCACG |
| | | AGGACCCCGAAGTCAAGTTCAACTGGTACGTGGATGGCGTCGAAGTGC |
| | | ATAATGCCAAGACCAAACCCCGGGAGGAACAGTACAACTCTACCTATA |
| | | GAGTCGTGAGTGTCCTGACAGTGCTGCACCAGGACTGGCTGAATGGGA |
| | | AGGAGTATAAGTGTAAAGTGAGCAACAAAGCCCTGCCCGCCCCAATCG |
| | | AAAAAACAATCTCTAAAGCAAAAGGACAGCCTCGCGAACCACAGGTCT |
| | | ACGTCTACCCCCCATCAAGAGATGAACTGACAAAAAATCAGGTCTCTCT |
| | | GACATGCCTGGTCAAAGGATTCTACCCTTCCGACATCGCCGTGGAGTGG |
| | | GAAAGTAACGGCCAGCCCGAGAACAATTACAAGACCACACCCCCTGTC |
| | | CTGGACTCTGATGGGAGTTTCGCTCTGGTGTCAAAGCTGACCGTCGATA |
| | | AAAGCCGGTGGCAGCAGGGCAATGTGTTTAGCTGCTCCGTCATGCACGA |
| | | AGCCCTGCACAATCACTACACACAGAAGTCCCTGAGCCTGAGCCCTGGC |
| 280 | 4561 | DIQMTQSPSSLSASVGDRVTITCRASQDVNTAVAWYQQKPGKAPKLLIYSA |
| | | SFLYSGVPSRFSGSRSGTDFTLTISSLQPEDFATYYCQQHYTTPPTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 281 | 4561 | GATATTCAGATGACCCAGTCCCCTAGCTCCCTGTCCGCTTCTGTGGGCG |
| | | ACAGGGTCACTATCACCTGCCGCGCATCTCAGGATGTGAACACCGCAGT |
| | | CGCCTGGTACCAGCAGAAGCCTGGGAAAGCTCCAAAGCTGCTGATCTA |
| | | CAGTGCATCATTCCTGTATTCAGGAGTGCCCAGCCGGTTTAGCGGCAGC |
| | | AGATCTGGCACCGACTTCACACTGACTATCTCTAGTCTGCAGCCTGAGG |
| | | ATTTTGCCACATACTATTGCCAGCAGCACTATACCACACCCCCTACTTTC |
| | | GGCCAGGGGACCAAAGTGGAGATCAAGCGAACTGTGGCCGCTCCAAGC |
| | | GTCTTCATTTTTCCACCCAGCGACGAACAGCTGAAATCCGGCACAGCTT |
| | | CTGTGGTCTGTCTGCTGAACAACTTCTACCCCAGAGAGGCCAAAGTGCA |
| | | GTGGAAGGTCGATAACGCTCTGCAGAGTGGCAACAGCCAGGAGAGCGT |
| | | GACAGAACAGGACTCCAAAGATTCTACTTATAGTCTGTCAAGCACCCTG |
| | | ACACTGAGCAAGGCAGACTACGAAAAGCATAAAGTGTATGCCTGTGAG |
| | | GTGACCCATCAGGGGCTGTCTTCTCCCGTGACCAAGTCTTTCAACCGAG |
| | | GCGAATGT |
| 282 | 8659 | QVQLVQSGAEVKKPGASVRVSCRASGYIFTESGITWVRQAPGQGLEWMG |
| | | WISGYSGDTKYAQKLQGRVTMTKDTSTTTAYMELRSLRYDDTAVYYCAR |
| | | DVQYSGSYLGAYYFDYWSPGTLVTVSSASTKGPSVFPLAPSSKSTSGGTAA |
| | | LGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPRE |
| | | PQVYVLPPSRDELTKNQVSLLCLVKGFYPSDIAVEWESNGQPENNYLTWPP |
| | | VLDSDGSFFLYSKLTVDKSRWQQGNVFSCSVMHEALHNHYTQKSLSLSPG |
| 283 | 8659 | CAGGTGCAGCTGGTCCAGTCCGGGGCTGAAGTGAAAAAACCTGGGGCA |
| | | TCCGTGCGGGTGTCATGTCGGGCAAGCGGGTATATCTTTACTGAGTCTG |
| | | GAATCACCTGGGTGAGGCAGGCTCCCGGACAGGGACTGGAATGGATGG |
| | | GATGGATTTCTGGATACAGTGGCGACACAAAGTATGCACAGAAACTGC |
| | | AGGGCCGCGTCACCATGACAAAGGATACTTCAACCACAACTGCCTACAT |
| | | GGAGCTGCGGAGCCTGAGATATGACGATACAGCCGTGTACTATTGCGCC |
| | | CGGGACGTGCAGTACAGCGGGTCCTACCTGGGGGCATACTACTTCGATT |
| | | ACTGGTCACCTGGAACTCTGGTCACCGTCTCTTCAGCTAGCACCAAGGG |
| | | CCCTTCTGTGTTTCCACTGGCACCCTCAAGCAAAAGCACCTCCGGAGGA |
| | | ACAGCAGCACTGGGATGTCTGGTCAAGGACTATTTCCCCGAGCCTGTGA |
| | | CCGTCTCATGGAATAGCGGCGCACTGACTAGTGGGGTGCACACCTTTCC |
| | | CGCCGTCCTGCAGTCCTCTGGGCTGTACAGCCTGAGTTCAGTGGTCACA |
| | | GTGCCAAGCTCCTCTCTGGGAACTCAGACCTATATCTGCAACGTCAATC |
| | | ATAAACCCAGCAACACAAAGGTCGACAAGAAAGTGGAGCCCAAGAGCT |
| | | GTGATAAAACTCATACCTGCCCACCTTGTCCGGCGCCAGAACTGCTGGG |
| | | AGGACCAAGCGTGTTCCTGTTTCCACCCAAGCCTAAAGACACCCTGATG |
| | | ATTTCCCGGACTCCTGAGGTCACCTGCGTGGTCGTGGACGTGTCTCACG |
| | | AGGACCCCGAAGTCAAGTTCAACTGGTACGTGGATGGCGTCGAAGTGC |
| | | ATAATGCCAAGACCAAACCCCGGGAGGAACAGTACAACTCTACCTATA |
| | | GAGTCGTGAGTGTCCTGACAGTGCTGCACCAGGACTGGCTGAATGGGA |
| | | AGGAGTATAAGTGTAAAGTGAGCAACAAAGCCCTGCCCGCCCCAATCG |
| | | AAAAAACAATCTCTAAAGCAAAAGGACAGCCTCGCGAACCACAGGTCT |
| | | ACGTGCTGCCCCCTAGCCGCGACGAACTGACTAAAAATCAGGTCTCTCT |
| | | GCTGTGTCTGGTCAAAGGATTCTACCCTTCCGACATCGCCGTGGAGTGG |
| | | GAAAGTAACGGCCAGCCCGAGAACAATTACCTGACCTGGCCCCCTGTG |
| | | CTGGACTCTGATGGGAGTTTCTTTCTGTATTCAAAGCTGACAGTCGATA |
| | | AAAGCCGGTGGCAGCAGGGCAATGTGTTCAGCTGCTCCGTCATGCACG |
| | | AAGCACTGCACAACCATTACACTCAGAAGTCCCTGTCCCTGTCACCTGG |
| | | C |
| 284 | 16855 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGGGGGQVQLVQSG |
| | | AEVKKPGASVKVSCKASGYSFTGYTMNWVRQAPGQGLEWMGLITPYNGA |
| | | SSYNQKFRGKATMTVDTSTSTVYMELSSLRSEDTAVYYCARGGYDGRGFD |
| | | YWGQGTLVTVSSGGGGSGGGGSGGGGSDIQMTQSPSSLSASVGDRVTITCS |
| | | ASSSVSYMHWYQQKSGKAPKLLIYDTSKLASGVPSRFSGSGSGTDFTLTISS |
| | | LQPEDFATYYCQQWSKHPLTFGQGTKLEIK |
| 285 | 16855 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCTCAGGTGTACGTGTATCCTCCAAGCAGAGACGAG |
| | | CTGACCAAGAACCAGGTGTCCCTGACATGTCTGGTGAAGGGCTTTTACC |
| | | CCTCTGATATCGCCGTGGAGTGGGAGAGCAATGGCCAGCCTGAGAACA |
| | | ATTATAAGACCACACCCCCTGTGCTGGACAGCGATGGCTCCTTCGCCCT |
| | | GGTGAGCAAGCTGACCGTGGACAAGTCCAGGTGGCAGCAGGGCAACGT |
| | | GTTTTCCTGCTCTGTGATGCACGAGGCCCTGCACAATCACTACACACAG |
| | | AAGAGCCTGAGCTTAAGCCCAGGAGGAGGAGGAGGACAGGTGCAGCTG |
| | | GTGCAGAGCGGAGCCGAGGTGAAGAAGCCTGGGGCCAGCGTGAAGGTG |
| | | TCTTGCAAGGCCTCTGGCTACAGCTTCACAGGCTATACCATGAACTGGG |
| | | TGCGGCAGGCCCCCGGACAGGGACTGGAGTGGATGGGCCTGATCACAC |
| | | CTTACAACGGGGCCAGCTCCTATAATCAGAAGTTTAGGGGCAAGGCCA |
| | | CCATGACAGTGGACACCTCCACATCTACCGTGTACATGGAGCTGTCTAG |
| | | CCTGCGCTCCGAGGATACCGCCGTGTACTATTGCGCCAGAGGCGGATAC |
| | | GACGGCAGAGGCTTCGATTATTGGGGCCAGGGCACACTGGTGACCGTG |
| | | TCCTCTGGAGGAGGAGGCAGCGGCGGAGGAGGCTCCGGCGGCGGCGGC |
| | | TCTGACATCCAGATGACACAGTCTCCAAGCTCCCTGAGCGCCTCCGTGG |
| | | GCGATCGGGTGACAATCACCTGTAGCGCCTCTAGCTCCGTGTCCTACAT |
| | | GCACTGGTATCAGCAGAAGTCCGGCAAGGCCCCAAAGCTGCTGATCTAT |
| | | GACACCAGCAAGCTGGCCTCCGGAGTGCCATCTAGGTTCTCTGGCAGCG |
| | | GCTCCGGCACAGACTTTACACTGACCATCTCTAGCCTGCAGCCTGAGGA |
| | | TTTCGCCACCTACTATTGTCAGCAGTGGAGCAAGCACCCACTGACATTT |
| | | GGCCAGGGCACCAAGCTGGAGATCAAG |
| 286 | 16861 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGGGGGQVQLVQSG |
| | | AEVVKPGASVKMSCKASGYTFTGYNIHWVKQAPGQGLEWIGAIYPGNGD |
| | | TSYKQKFRGRATLTADTSTSTVYMELSSLRSEDSAVYYCARGETARATFAY |
| | | WGQGTLVTVSSGGGGSGGGGSGGGGSDIQMTQSPSSLSASVGDRVTITCSA |
| | | SQDIGNFLNWYQQKPGKTVKVLIYYTSSLYSGVPSRFSGSGSGTDYTLTISS |
| | | LQPEDFATYYCQQYSKLPLTFGQGTKLELK |
| 287 | 16861 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAACCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCTCAGGTGTACGTGTATCCTCCAAGCAGAGACGAG |
| | | CTGACCAAGAACCAGGTGTCCCTGACATGTCTGGTGAAGGGCTTTTACC |
| | | CCTCTGATATCGCCGTGGAGTGGGAGAGCAATGGCCAGCCTGAGAACA |
| | | ATTATAAGACCACACCCCCTGTGCTGGACAGCGATGGCTCCTTCGCCCT |
| | | GGTGAGCAAGCTGACCGTGGACAAGTCCAGGTGGCAGCAGGGCAACGT |
| | | GTTTTCCTGCTCTGTGATGCACGAGGCCCTGCACAATCACTACACACAG |
| | | AAGAGCCTGAGCTTAAGCCCTGGAGGAGGAGGAGGACAGGTGCAGCTG |
| | | GTGCAGTCTGGAGCCGAGGTGGTGAAGCCAGGGGCCAGCGTGAAGATG |
| | | TCCTGCAAGGCCTCTGGCTACACCTTCACAGGCTATAACATCCACTGGG |
| | | TGAAGCAGGCCCCCGGACAGGGACTGGAGTGGATCGGAGCCATCTACC |
| | | CTGGCAATGGCGACACAAGCTATAAGCAGAAGTTTCGGGGCAGAGCCA |
| | | CCCTGACAGCCGATACCTCCACATCTACCGTGTACATGGAGCTGAGCTC |
| | | CCTGCGGTCCGAGGACTCTGCCGTGTACTATTGCGCCAGGGGCGAGACA |
| | | GCAAGGGCCACCTTCGCCTATTGGGGACAGGGCACACTGGTGACCGTGT |
| | | CTAGCGGCGGCGGCGGCTCTGGAGGAGGAGGCAGCGGCGGAGGAGGCT |
| | | CCGATATCCAGATGACCCAGTCCCCATCCTCTCTGAGCGCCTCCGTGGG |
| | | CGACCGGGTGACAATCACCTGTTCTGCCAGCCAGGATATCGGCAACTTC |
| | | CTGAATTGGTACCAGCAGAAGCCCGGCAAGACAGTGAAGGTGCTGATC |
| | | TACTATACCAGCTCCCTGTACAGCGGCGTGCCTTCCAGGTTTTCCGGCTC |
| | | TGCAGCGGCACAGACTATACACTGACCATCTCTAGCCTGCAGCCAGAG |
| | | GATTTCGCCACCTACTATTGTCAGCAGTACAGCAAGCTGCCCCTGACAT |
| | | TTGGCCAGGGCACCAAGCTGGAGCTGAAG |
| 288 | 16863 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGGGGGEVQLVESG |
| | | GGLVQPGGSLRLSCAASGFSFSDFAMSWVRQAPGKGLEWVATIGRVAFHT |
| | | YYPDSMKGRFTISRDNSKNTLYLQMNSLRAEDTAVYYCARHRGFDVGHFD |
| | | FWGQGTLVTVSSGGGGSGGGGSGGGGSDIQMTQSPSSLSASVGDRVTITCR |
| | | SSETLVHSSGNTYLEWYQQKPGKAPKLLIYRVSNRFSGVPSRFSGSGSGTDF |
| | | TLTISSLQPEDFATYYCFQGSFNPLTFGQGTKVEIK |
| 289 | 16863 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCTCAGGTGTACGTGTATCCTCCAAGCAGAGACGAG |
| | | CTGACCAAGAACCAGGTGTCCCTGACATGTCTGGTGAAGGGCTTTTACC |
| | | CCTCTGATATCGCCGTGGAGTGGGAGAGCAATGGCCAGCCTGAGAACA |
| | | ATTATAAGACCACACCCCCTGTGCTGGACAGCGATGGCTCCTTCGCCCT |
| | | GGTGAGCAAGCTGACCGTGGACAAGTCCAGGTGGCAGCAGGGCAACGT |
| | | GTTTTCCTGCTCTGTGATGCACGAGGCCCTGCACAATCACTACACACAG |
| | | AAGAGCCTGAGCTTAAGCCCTGGAGGAGGAGGAGGAGAGGTGCAGCTG |
| | | GTGGAGAGCGGCGGCGGCCTGGTGCAGCCAGGAGGCTCTCTGAGGCTG |
| | | AGCTGCGCAGCCTCCGGCTTCTCCTTTTCTGACTTCGCCATGTCCTGGGT |
| | | GCGGCAGGCCCCCGGCAAGGGACTGGAGTGGGTGGCCACCATCGGCAG |
| | | AGTGGCCTTCCACACATACTATCCTGACTCCATGAAGGGCCGGTTTACC |
| | | ATCTCTAGAGATAACAGCAAGAATACACTGTACCTGCAGATGAACTCCC |
| | | TGCGCGCCGAGGATACCGCCGTGTACTATTGCGCAAGGCACAGAGGCTT |
| | | TGACGTGGGACACTTCGATTTTTGGGGCCAGGGCACCCTGGTGACAGTG |
| | | AGCAGCGGAGGAGGAGGCTCCGGCGGCGGAGGCTCTGGAGGAGGAGG |
| | | CAGCGACATCCAGATGACCCAGAGCCCATCTAGCCTGAGCGCCTCCGTG |
| | | GGCGATAGGGTGACCATCACATGTCGCTCCTCTGAGACCCTGGTGCACA |
| | | GCTCCGGCAACACATACCTGGAGTGGTATCAGCAGAAGCCCGGCAAGG |
| | | CCCCTAAGCTGCTGATCTATAGGGTGTCCAATCGCTTCTCTGGAGTGCC |
| | | AAGCCGGTTTTCTGGCAGCGGCTCCGGAACCGACTTCACCCTGACAATC |
| | | TCTAGCCTGCAGCCAGAGGATTTTGCCACATACTATTGTTTCCAGGGCT |
| | | CCTTTAATCCCCTGACCTTCGGCCAGGGCACAAAGGTGGAGATCAAG |
| 290 | 18512 | QVQLQESGPGLVQPTQSLSITCTVSGFSLISYGVHWVRQSPGKGLEWLGVI |
| | | WSGGSTDYNAAFISRLSISKDNSKSQVFFKMNSLQADDTAIYYCARNPLTA |
| | | TVMDYWGQGTSVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVYPPSRD |
| | | ELTKNQVSLTCLVKGFYPSDIAVEWESNGQPENNYKTTPPVLDSDGSFALV |
| | | SKLTVDKSRWQQGNVFSCSVMHEALHNHYTQKSLSLSPGGGGGQEQLVES |
| | | GGGLVQPEGSLTLTCTASKFSFSSLYYMCWVRQAPGKGLEWIACVYGGSS |
| | | GNTYYASWAKGRFTISKASSTTVTLQLTSLTAADTATYFCARFDVDGSGFN |
| | | LWGPGTLVTVSSGGGGSGGGGSGGGGSDIVMTQTPSSVSAAVGGTVTIKC |
| | | QASQTIGSSLAWYQQKPGQPPKLLIYRASTLASGVSSRFRGSGSGTEYTLTIS |
| | | DLECADAATYYCQWTDYGYIYIWAFGGGTEVVVK |
| 291 | 18512 | CAGGTGCAGCTGCAGGAGAGCGGACCAGGACTGGTGCAGCCTACACAG |
| | | TCTCTGAGCATCACCTGCACAGTGTCTGGCTTCACCCTGATCTCCTACGG |
| | | AGTGCACTGGGTGAGGCAGTCCCCTGGCAAGGGACTGGAGTGGCTGGG |
| | | CGTGATCTGGTCTGGCGGCACCACCGACTATAACGCCGCCTTTATCTCC |
| | | CGCCTGTCCATCTCTAAGGATAACAGCAAGTCCCAGGTGTTCTTTAAGA |
| | | TGAACAGCCTGCAGGCCGACGATACAGCCATCTACTATTGTGCCCGGAA |
| | | TCCCCTGACCGCCACAGTGATGGACTACTGGGGCCAGGGCACCTCTGTG |
| | | ACAGTGAGCTCCGCTAGCACAAAGGGCCCCTCCGTGTTTCCTCTGGCCC |
| | | CATCCTCTAAGTCCACCTCTGGAGGAACAGCCGCCCTGGGCTGTCTGGT |
| | | GAAGGATTACTTCCCTGAGCCAGTGACCGTGTCCTGGAACTCTGGGGCC |
| | | CTGACCAGCGGAGTGCACACATTTCCCGCCGTGCTGCAGAGCTCCGGAC |
| | | TGTACTCCCTGTCTAGCGTGGTGACCGTGCCTTCCTCTAGCCTGGGCACC |
| | | CAGACATATATCTGCAACGTGAATCACAAGCCTTCCAATACAAAGGTCG |
| | | ACAAGAAGGTGGAGCCAAAGTCTTGTGATAAGACCCACACATGCCCAC |
| | | CTTGTCCGGCGCCAGAGGCCGCCGGAGGACCAAGCGTGTTCCTGTTTCC |
| | | ACCCAAGCCCAAGGACACCCTGATGATCTCCCGGACCCCAGAGGTGAC |
| | | ATGCGTGGTGGTGAGCGTGTCCCACGAGGACCCCGAGGTGAAGTTTAA |
| | | CTGGTACGTGGATGGCGTGGAGGTGCACAATGCCAAGACAAAGCCCCG |
| | | GGAGGAGCAGTACAATTCTACCTATAGAGTGGTGAGCGTGCTGACAGT |
| | | GCTGCACCAGGATTGGCTGAACGGCAAGGAGTATAAGTGTAAGGTGAG |
| | | CAATAAGGCCCTGCCAGCCCCCATCGAGAAGACCATCTCCAAGGCCAA |
| | | GGGCCAGCCTCGCGAGCCCCAGGTGTACGTGTATCCCCCTAGCAGAGAC |
| | | GAGCTGACAAAGAACCAGGTGTCCCTGACCTGCCTGGTGAAGGGCTTCT |
| | | ATCCCTCTGATATCGCCGTGGAGTGGGAGAGCAATGGCCAGCCTGAGA |
| | | ACAATTACAAGACCACACCACCCGTGCTGGACAGCGATGGCTCCTTCGC |
| | | CCTGGTGAGCAAGCTGACAGTGGACAAGTCCAGGTGGCAGCAGGGCAA |
| | | CGTGTTTTCTTGTAGCGTGATGCACGAGGCCCTGCACAATCACTATACC |
| | | CAGAAGTCCCTGTCTCTGAGCCCAGGAGGAGGAGGAGGACAGGAGCAG |
| | | CTGGTGGAGTCTGGCGGCGGCCTGCAGCAGCCAGAGGGCTCCCTGACCC |
| | | TGACATGCACCGCCTCTAAGTTCAGCTTTAGCTCCCTGTACTATATGTGC |
| | | TGGGTGAGGCAGGCCCCCGGCAAGGGACTGGAGTGGATCGCCTGCGTG |
| | | TATGGCGGCTCTAGCGGCAACACCTACTATGCCTCCTGGGCCAAGGGCC |
| | | GCTTCACAATCTCTAAGGCCTCCTCTACCACAGTGACCCTGCAGCTGAC |
| | | AAGCCTGACCGCCGCCGACACAGCCACCTACTTCTGTGCCCGGTTTGAC |
| | | GTGGATGGCTCCGGCTTTAATCTGTGGGGCCCTGGCACACTGGTGACCG |
| | | TGAGCTCCGGAGGAGGAGGCACCGGAGGAGGAGGCTCCGGCGGCGGC |
| | | GGCTCTGATATCGTGATGACACAGACCCCATCTAGCGTGAGCGCCGCCG |
| | | TGGGAGGCACAGTGACCATCAAGTGCCAGGCCTCCCAGACCATCGGCT |
| | | CCTCTCTGGCCTGGTATCAGCAGAAGCCTGGCCAGCCTCCAAAGCTGCT |
| | | GATCTACAGAGCCTCCACACTGGCCTCTGGCGTGAGCTCCCGGTTCAGA |
| | | GGCTCCGGCTCTGGAACCGAGTACACACTGACCATCAGCGACCTGGAGT |
| | | GCGCAGATGCAGCAACATACTATTGTCAGTGGACCGATTACGCCTATAT |
| | | CTACATCTGGGCCTTTGGCGGAGGAACCGAGGTGGTGGTGAAG |
| 292 | 16866 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGGGGGQVELVQSG |
| | | AEVKKPGESLKISCKGSGYSFTSYWIGWVRQAPGKGLEWMGIIDPGDSRTR |
| | | YSPSFQGQVTISADKSISTAYLQWSSLKASDTAMYYCARGQLYGGTYMDG |
| | | WGQGTLVTVSSVEGGSGGSGGSGGSGGVDDIALTQPASVSGSPGQSITISCT |
| | | GTSSDIGGYNSVSWYQQHPGKAPKLMIYGVNNRPSGVSNRFSGSKSGNTA |
| | | SLTISGLQAEDEADYYCSSYDIESATPVFGGGTKLTVL |
| 293 | 16866 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCTCAGGTGTACGTGTATCCTCCAAGCAGAGACGAG |
| | | CTGACCAAGAACCAGGTGTCCCTGACATGTCTGGTGAAGGGCTTTTACC |
| | | CCTCTGATATCGCCGTGGAGTGGGAGAGCAATGGCCAGCCTGAGAACA |
| | | ATTATAAGACCACACCCCCTGTGCTGGACAGCGATGGCTCCTTCGCCCT |
| | | GGTGAGCAAGCTGACCGTGGACAAGTCCAGGTGGCAGCAGGGCAACGT |
| | | GTTTTCCTGCTCTGTGATGCACGAGGCCCTGCACAATCACTACACACAG |
| | | AAGAGCCTGAGCTTAAGCCCAGGAGGAGGAGGAGGACAGGTGGAGCT |
| | | GGTGCAGTCCGGAGCCGAGGTGAAGAAGCCTGGCGAGTCTCTGAAGAT |
| | | CAGCTGCAAGGGCTCTGGCTACAGCTTCACCTCCTATTGGATCGGATGG |
| | | GTGCGGCAGGCCCCTGGCAAGGGACTGGAGTGGATGGGCATCATCGAC |
| | | CCTGGCGATTCTCGGACAAGATACTCTCCAAGCTTTCAGGGCCAGGTGA |
| | | CCATCAGCGCCGACAAGTCCATCTCTACAGCCTATCTGCAGTGGAGCTC |
| | | CCTGAAGGCCAGCGATACCGCCATGTACTATTGCGCCAGGGGCCAGCTG |
| | | TACGGAGGAACATATATGGACGGATGGGGACAGGGCACCCTGGTGACA |
| | | GTGTCTAGCGTGGAGGGAGGCTCTGGAGGCAGCGGAGGCTCCGGAGGC |
| | | TCTGGAGGAGTGGACGATATCGCCCTGACCCAGCCAGCCAGCGTGTCCG |
| | | GCTCTCCAGGCCAGTCCATCACAATCTCTTGTACCGGCACATCCTCTGAT |
| | | ATCGGCGGCTACAACAGCGTGTCCTGGTATCAGCAGCACCCCGGCAAG |
| | | GCCCCTAAGCTGATGATCTACGGCGTGAACAATAGGCCAAGCGGCGTG |
| | | TCCAACCGCTTCTCTGGCAGCAAGTCCGGCAATACCGCCAGCCTGACAA |
| | | TCTCCGGACTGCAGGCAGAGGACGAGGCAGATTACTATTGTAGCTCCTA |
| | | TGACATCGAGTCCGCCACCCCCGTGTTTGGAGGAGGCACCAAGCTGACA |
| | | GTGCTG |
| 294 | 16870 | EVQLVQSGAEVKKPGESLRISCKGSGYSFSTYWISWVRQMPGKGLEWMGK |
| | | IYPGDSYTNYSPSFQGQVTISADKSISTAYLQWSSLKASDTAMYYCARGYGI |
| | | FDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGQVELVQSG |
| | | AEVKKPGESLKISCKGSGYSFTSYWIGWVRQAPGKGLEWMGIIDPGDSRTR |
| | | YSPSFQGQVTISADKSISTAYLQWSSLKASDTAMYYCARGQLYGGTYMDG |
| | | WGQGTLVTVSSVEGGSGGSGGSGGSGGVDDIALTQPASVSGSPGQSITISCT |
| | | GTSSDIGGYNSVSWYQQHPGKAPKLMIYGVNNRPSGVSNRFSGSKSGNTA |
| | | SLTISGLQAEDEADYYCSSYDIESATPVFGGGTKLTVL |
| 295 | 16870 | GAGGTGCAGCTGGTGCAGAGCGGAGCAGAGGTGAAGAAGCCAGGCGA |
| | | GTCCCTGAGGATCTCTTGCAAGGGCAGCGGCTACAGCTTCTCCACCTAT |
| | | TGGATCTCCTGGGTGCGCCAGATGCCTGGCAAGGGACTGGAGTGGATG |
| | | GGCAAGATCTACCCCGGCGACAGCTACACAAACTATTCTCCTAGCTTTC |
| | | AGGGCCAGGTGACCATCTCCGCCGATAAGTCCATCTCTACAGCCTATCT |
| | | GCAGTGGAGCTCCCTGAAGGCCAGCGACACCGCCATGTACTATTGCGCC |
| | | AGAGGCTACGGCATCTTCGATTATTGGGGCCAGGGCACCCTGGTGACAG |
| | | TGTCTAGCGCTAGCACAAAGGGCCCAAGCGTGTTTCCTCTGGCCCCATC |
| | | CTCTAAGAGCACCTCCGGAGGAACAGCCGCCCTGGGCTGTCTGGTGAA |
| | | GGACTACTTCCCTGAGCCAGTGACCGTGTCCTGGAACTCTGGGGCCCTG |
| | | ACCAGCGGAGTGCACACATTTCCCGCCGTGCTGCAGAGCTCCGGACTGT |
| | | ACTCCCTGTCTAGCGTGGTGACCGTGCCTTCCTCTAGCCTGGGCACCCA |
| | | GACATATATCTGCAACGTGAATCACAAGCCTTCTAATACAAAGGTCGAC |
| | | AAGAAGGTGGAGCCAAAGAGCTGTGATAAGACCCACACATGCCCACCT |
| | | TGTCCGGCGCCAGAGGCCGCCGGAGGACCAAGCGTGTTCCTGTTTCCAC |
| | | CCAAGCCCAAGGACACCCTGATGATCTCCAGGACCCCTGAGGTGACAT |
| | | GCGTGGTGGTGTCTGTGAGCCACGAGGACCCAGAGGTGAAGTTCAACT |
| | | GGTACGTGGATGGCGTGGAGGTGCACAATGCCAAGACAAAGCCCAGAG |
| | | AGGAGCAGTACAATTCCACCTATCGCGTGGTGTCTGTGCTGACAGTGCT |
| | | GCACCAGGATTGGCTGAACGGCAAGGAGTATAAGTGCAAGGTGTCCAA |
| | | TAAGGCCCTGCCAGCCCCCATCGAGAAGACCATCTCTAAGGCCAAGGG |
| | | CCAGCCTCGCGAACCTCAGGTGTACGTGCTGCCCCCTTCCAGGGACGAG |
| | | CTGACCAAGAACCAGGTGTCTCTGCTGTGCCTGGTGAAGGGCTTCTATC |
| | | CTTCTGATATCGCCGTGGAGTGGGAGAGCAATGGCCAGCCAGAGAACA |
| | | ATTACCTGACCTGGCCACCCGTGCTGGACTCCGATGGCTCTTTCTTTCTG |
| | | TATAGCAAGCTGACAGTGGACAAGTCCAGATGGCAGCAGGGCAACGTG |
| | | TTTTCTTGCAGCGTGATGCACGAGGCCCTGCACAATCACTACACACAGA |
| | | AGTCCCTGAGCTTAAGCCCAGGAGGAGGAGGAGGACAGGTGGAGCTGG |
| | | TGCAGAGCGGAGCCGAGGTGAAGAAGCCTGGCGAGAGCCTGAAGATCT |
| | | CCTGTAAGGGCAGCGGCTACTCCTTCACCTCTTATTGGATCGGATGGGT |
| | | GCGGCAGGCCCCCGGCAAGGGCCTGGAGTGGATGGGCATCATCGACCC |
| | | AGGCGATAGCCGGACAAGATACTCCCCCTCTTTTCAGGGCCAGGTGACC |
| | | ATCTCCGCCGACAAGAGCATCTCCACAGCCTATCTGCAGTGGAGCTCCC |
| | | TGAAGGCCTCCGATACCGCCATGTACTATTGCGCCAGAGGCCAGCTGTA |
| | | CGGCGGCACATATATGGACGGATGGGGACAGGGCACCCTGGTGACAGT |
| | | GTCTAGCGTGGAGGGAGGCAGCGGAGGCTCCGGAGGCTCTGGAGGCAG |
| | | CGGAGGAGTGGACGATATCGCCCTGACCCAGCCCGCCTCTGTGAGCGG |
| | | CTCCCCTGGCCAGTCTATCACAATCAGCTGTACCGGCACATCCTCTGAT |
| | | ATCGGCGGCTACAACTCTGTGAGCTGGTATCAGCAGCACCCTGGCAAGG |
| | | CCCCAAAGCTGATGATCTACGGCGTGAACAATAGGCCATCCGGCGTGTC |
| | | TAACCGCTTCTCCGGCTCTAAGAGCGGCAATACCGCCTCCCTGACAATC |
| | | TCTGGACTGCAGGCAGAGGACGAGGCAGATTACTATTGTAGCTCCTATG |
| | | ATATCGAGAGCGCCACCCCCGTGTTTGGAGGAGGCACCAAGCTGACAG |
| | | TGCTG |
| 296 | 11761 | QVQLVQSGAEVVKPGASVKISCKASGYTFTGYFMNWVKQSPGQSLEWIGR |
| | | IHPYDGDTFYNQKFQGKATLTVDKSSNTAHMELLSLTSEDFAVYYCTRYD |
| | | GSRAMDYWGQGTTVTVSSGGGGSGGGGSGGGGSDIVLTQSPLSLAVSLGQ |
| | | PAIISCKASQSVSFAGTSLMHWYHQKPGQQPRLLIYRASNLEAGVPDRFSGS |
| | | GSKTDFTLTISPVEAEDAATYYCQQSREYPYTFGGGTKLEIKAAEPKSSDKT |
| | | HTCPPCPAPELLGGPSVFLEPPKPKDTLMISRTPEVTCVVVDVSHEDPEVKF |
| | | NWYVDGVEVHNAKTKPREEQYNSTYRVVSVLTVLHQDWLNGKEYKCKV |
| | | SNKALPAPIEKTISKAKGQPREPQVYVLPPSRDELTKNQVSLLCLVKGFYPS |
| | | DIAVEWESNGQPENNYLTWPPVLDSDGSFFLYSKLTVDKSRWQQGNVFSC |
| | | SVMHEALHNHYTQKSLSLSPG |
| 297 | 11761 | CAGGTGCAGCTGGTGCAGAGCGGAGCCGAGGTGGTGAAGCCAGGGGCC |
| | | AGCGTGAAGATCAGCTGCAAGGCCTCCGGCTACACCTTCACAGGCTACT |
| | | TCATGAACTGGGTGAAGCAGTCTCCTGGCCAGAGCCTGGAGTGGATCG |
| | | GCAGAATCCACCCATACGACGGCGATACCTTCTATAACCAGAAGTTTCA |
| | | GGGCAAGGCCACCCTGACAGTGGACAAGAGCTCCAATACCGCCCACAT |
| | | GGAGCTGCTGTCCCTGACATCTGAGGATTTCGCCGTGTACTATTGCACC |
| | | CGGTACGACGGCTCCAGAGCCATGGATTATTGGGGCCAGGGCACCACA |
| | | GTGACAGTGTCTAGCGGAGGAGGAGGCTCCGGAGGAGGAGGCTCTGGC |
| | | GGCGGCGGCAGCGACATCGTGCTGACCCAGAGCCCACTGTCCCTGGCC |
| | | GTGTCCCTGGGCCAGCCCGCCATCATCTCTTGTAAGGCCTCCCAGAGCG |
| | | TGAGCTTCGCCGGGACCAGCCTGATGCACTGGTACCACCAGAAGCCCG |
| | | GCCAGCAGCCCAGACTGCTGATCTATAGGGCCTCCAATCTGGAGGCCGG |
| | | AGTGCCAGACCGGTTCTCCGGCTCTGGCAGCAAGACCGACTTCACCCTG |
| | | ACAATCAGCCCTGTGGAGGCAGAGGATGCAGCAACATACTATTGTCAG |
| | | CAGTCCAGGGAGTACCCATATACCTTTGGCGGCGGCACAAAGCTGGAG |
| | | ATCAAGGCGGCCGAGCCTAAAAGTAGCGATAAAACCCATACCTGCCCC |
| | | CCCTGCCCGGCGCCAGAACTGCTGGGAGGACCAAGCGTGTTCCTGTTTC |
| | | CACCCAAGCCTAAAGACACCCTGATGATTTCCCGGACTCCTGAGGTCAC |
| | | CTGCGTGGTCGTGGACGTGTCTCACGAGGACCCCGAAGTCAAGTTCAAC |
| | | TGGTACGTGGATGGCGTCGAAGTGCATAATGCCAAGACCAAACCCCGG |
| | | GAGGAACAGTACAACTCTACCTATAGAGTCGTGAGTGTCCTGACAGTGC |
| | | TGCACCAGGACTGGCTGAATGGGAAGGAGTATAAGTGTAAAGTGAGCA |
| | | ACAAAGCCCTGCCCGCCCcAATcGAAAAAACAATCTCTAAAGCAAAAG |
| | | GACAGCCTCGCGAACCACAGGTCTACGTGCTGCCCCCTAGCCGCGACGA |
| | | ACTGACTAAAAATCAGGTCTCTCTGCTGTGTCTGGTCAAAGGATTCTAC |
| | | CCTTCCGACATCGCCGTGGAGTGGGAAAGTAACGGCCAGCCCGAGAAC |
| | | AATTACCTGACCTGGCCCCCTGTGCTGGACTCTGATGGGAGTTTCTTTCT |
| | | GTATTCAAAGCTGACAGTCGATAAAAGCCGGTGGCAGCAGGGCAATGT |
| | | GTTCAGCTGCTCCGTCATGCACGAAGCACTGCACAACCATTACACTCAG |
| | | AAGTCCCTGTCCCTGTCACCTGGC |

TABLE Y1
Sequences

| SEQ | Description | |
|---|---|---|
| ID | or clone | |
| NO: | number | Sequence |
| 344 | 12153 | EPKSSDKTHTCPPCPAPEAAGGPSVFLFPPKPKDTLMISRTPEVTCVVVSVS |
| | | HEDPEVKFNWYVDGVEVHNAKTKPREEQYNSTYRVVSVLTVLHQDWLN |
| | | GKEYKCKVSNKALPAPIEKTISKAKGQPREPQVYVLPPSRDELTKNQVSLL |
| | | CLVKGFYPSDIAVEWESNGQPENNYLTWPPVLDSDGSFFLYSKLTVDKSR |
| | | WQQGNVFSCSVMHEALHNHYTQKSLSLSPG |
| 345 | 20891 | DIQMTQSPSSLSASVGDRVTITCSASQGISNYLNWYQQKPGKAPKLLIYYTS |
| | | SLHSGVPSRFSGSGSGTDFTFTISSLQPEDIATYYCQQYSKFPWTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 346 | 20898 | EIVLTQSPATLSLSPGERATLSCKASENVGSYVSWYQQKPGQAPRLLIYGAS |
| | | NRYTGVPARFSGSGSGTDFTLTISSLEPEDFAVYYCGQSYSYPLTFGGGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 347 | 21663 | EVQLVESGGGLVQPGGSLRLSCAASGFTISNNYYMCWVRQAPGKGLEWIA |
| | | CIYGGISGRTYYADSAKGRFTISKDSSNTVYLQMNSLRAEDTAVYYCVRGY |
| | | VGTSNLWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHT |
| 348 | 21708 | EVQLVESGGGLIQPGGSLRISCAVSGFSLTTYAISWVRQAPGKGLEWLGIIW |
| | | PGGGTNYADSLKGRLTISKDNSKNTVYLQMNSLRTEDTAVYYCARGAGT |
| | | WYFDVWGQGTLVTVSSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTLMISRT |
| | | PEVTCVVVSVSHEDPEVKFNWYVDGVEVHNAKTKPREEQYNSTYRVVSV |
| | | LTVLHQDWLNGKEYKCKVSNKALPAPIEKTISKAKGQPREPQVYVLPPSRD |
| | | ELTKNQVSLLCLVKGFYPSDIAVEWESNGQPENNYLTWPPVLDSDGSFFLY |
| | | SKLTVDKSRWQQGNVFSCSVMHEALHNHYTQKSLSLSPG |
| 349 | 21709 | EVQLVESGGGLVQPGGSLRLSCAASGFTFSEFGMHWVRQAPGKGLEWVSY |
| | | ISSGGSTIYYADSVKGRFTISRDNAKNSLYLQMNSLRAEDTAVYYCARDW |
| | | VDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPG |
| 350 | 21828 | DIVMTQTPASVEAAVGGTVTIKCQASQSIYSSLAWYQQKPGQSPKLLIYDA |
| | | SHLASGVPSRFSGSRYGTEFTLTISGVQSDDAATYYCQGGWYSSAATYVPN |
| | | TFGGGTEVVVKGGGGSGGGGSGGGGSQEQLVESGGGLVQPEGSLTLTCKA |
| | | SGFTISNNYYMCWVRQAPGKGLEWIACIYGGISGRTYYASWAKGRFTISKT |
| | | SSTTVTLQMTSLTAADTATYFCVRGYVGTSNLWGPGTLVTVSSEPKSSDKT |
| | | HTCPPCPAPEAAGGPSVFLFPPKPKDTLMISRTPEVTCVVVSVSHEDPEVKF |
| | | NWYVDGVEVHNAKTKPREEQYNSTYRVVSVLTVLHQDWLNGKEYKCKV |
| | | SNKALPAPIEKTISKAKGQPREPQVYVYPPSRDELTKNQVSLTCLVKGFYPS |
| | | DIAVEWESNGQPENNYKTTPPVLDSDGSFALVSKLTVDKSRWQQGNVFSC |
| | | SVMHEALHNHYTQKSLSLSPG |
| 351 | 22043 | EVQLVESGGGLVQPGGSLRLSCAASGFTFSEFGMHWVRQAPGKGLEWVSY |
| | | ISSGGSTIYYADSVKGRFTISRDNAKNSLYLQMNSLRAEDTAVYYCARDW |
| | | VDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPG |
| 352 | 22775 | DIQMTQSPSSLSASVGDRVTITCQASQSIYSSLAWYQQKPGKAPKLLIYDAS |
| | | HLASGVPSRFSGSRYGTDFTLTISSVQPEDFATYYCQGGWYSSAATYVPNT |
| | | FGGGTKVEVKGGSGGGSGGGSGGGSGGGSGEVQLVESGGGLVQPGGSLR |
| | | LSCAASGFTISNNYYMCWVRQAPGKGLEWIACIYGGISGRTYYADSAKGR |
| | | FTISKDSSNTVYLQMNSLRAEDTAVYYCYRGYVGTSNLWGQGTLVTVSSG |
| | | GGGEVQLVESGGGLVQPGGSLRLSCAASGFTFSEFGMHWVRQAPGKGLE |
| | | WVSYISSGGSTIYYADSVKGRFTISRDNAKNSLYLQMNSLRAEDTAVYYCA |
| | | RDWVDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNV |
| | | NHKPSNTKVDKKVEPKSCDKTHTCPPCPAPEAAGGPSVFLFPPKPKDTLMIS |
| | | RTPEVTCVVVSVSHEDPEVKFNWYVDGVEVHNAKTKPREEQYNSTYRVV |
| | | SVLTVLHQDWLNGKEYKCKVSNKALPAPIEKTISKAKGQPREPQVYVYPPS |
| | | RDELTKNQVSLTCLVKGFYPSDIAVEWESNGQPENNYKTTPPVLDSDGSFA |
| | | LVSKLTVDKSRWQQGNVFSCSVMHEALHNHYTQKSLSLSPG |
| 353 | 22776 | EVQLVESGGGLVQPGGSLRLSCAASGFTFSEFGMHWVRQAPGKGLEWVSY |
| | | ISSGGSTIYYADSVKGRFTISRDNAKNSLYLQMNSLRAEDTAVYYCARDW |
| | | VDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGGGGGDIQMTQSPS |
| | | SLSASVGDRVTITCQASQSIYSSLAWYQQKPGKAPKLLIYDASHLASGVPSR |
| | | FSGSRYGTDFTLTISSVQPEDFATYYCQGGWYSSAATYVPNTFGGGTKVEV |
| | | KGGSGGGSGGGSGGGSGGGSGEVQLVESGGGLVQPGGSLRLSCAASGFTIS |
| | | NNYYMCWVRQAPGKGLEWIACIYGGISGRTYYADSAKGRFTISKDSSNTV |
| | | YLQMNSLRAEDTAVYYCVRGYVGTSNLWGQGTLVTVSS |
| 354 | 22777 | EVQLVESGGGLVQPGGSLRLSCAASGFTFSEFGMHWVRQAPGKGLEWVSY |
| | | ISSGGSTIYYADSVKGRFTISRDNAKNSLYLQMNSLRAEDTAVYYCARDW |
| | | VDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDEL |
| | | TKNQVSLLCLVKGFYPSDIAVEWESNGQPENNYLTWPPVLDSDGSFFLYSK |
| | | LTVDKSRWQQGNVFSCSVMHEALHNHYTQKSLSLSPGGGGGDIQMTQSPS |
| | | SLSASVGDRVTITCQASQSIYSSLAWYQQKPGKAPKLLIYDASHLASGVPSR |
| | | FSGSRYGTDFTLTISSVQPEDFATYYCQGGWYSSAATYVPNTFGGGTKVEV |
| | | KGGSGGGSGGGSGGGSGGGSGEVQLVESGGGLVQPGGSLRLSCAASGFTIS |
| | | NNYYMCWVRQAPGKGLEWIACIYGGISGRTYYADSAKGRFTISKDSSNTV |
| | | YLQMNSLRAEDTAVYYCVRGYVGTSNLWGQGTLVTVSS |
| 355 | 22782 | EPKSSDKTHTCPPCPAPEAAGGPSVFLFPPKPKDTLMISRTPEVTCVVVSVS |
| | | HEDPEVKFNWYVDGVEVHNAKTKPREEQYNSTYRVVSVLTVLHQDWLN |
| | | GKEYKCKVSNKALPAPIEKTISKAKGQPREPQVYVLPPSRDELTKNQVSLL |
| | | CLVKGFYPSDIAVEWESNGQPENNYLTWPPVLDSDGSFFLYSKLTVDKSR |
| | | WQQGNVFSCSVMHEALHNHYTQKSLSLSPGGGGGDIQMTQSPSSLSASVG |
| | | DRVTITCQASQSIYSSLAWYQQKPGKAPKLLIYDASHLASGVPSRFSGSRYG |
| | | TDFTLTISSVQPEDFATYYCQGGWYSSAATYVPNTFGGGTKVEVKGGSGG |
| | | GSGGGSGGGSGGGSGEVQLVESGGGLVQPGGSLRLSCAASGFTISNNYYM |
| | | CWVRQAPGKGLEWIACIYGGISGRTYYADSAKGRFTISKDSSNTVYLQMNS |
| | | LRAEDTAVYYCVRGYVGTSNLWGQGTLVTVSS |
| 356 | 22789 | EVQLVESGGGLVQPGGSLRLSCAASGFTFSEFGMHWVRQAPGKGLEWVSY |
| | | ISSGGSTIYYADSVKGRFTISRDNAKNSLYLQMNSLRAEDTAVYYCARDW |
| | | VDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGGGGGQVQLVQSG |
| | | AEVKKPGASVRVSCRASGYIFTESGITWVRQAPGQGLEWMGWISGYSGDT |
| | | KYAQKLQGRVTMTKDTSTTTAYMELRSLRYDDTAVYYCARDVQYSGSYL |
| | | GAYYFDYWSPGTLVTVSSGGGGSGGGGSGGGGSGGGQSVLTQPPSASGTP |
| | | GQRVTISCSGSSSNIGTNYVYWYQQFPGTAPKLLIYRSYQRPSGVPDRFSGS |
| | | KSGSSASLAISGLQSEDEADYYCATWDDSLDGWVFGGGTKLTVL |
| 357 | 22791 | EVQLVESGGGLVQPGGSLRLSCAASGFTFSEFGMHWVRQAPGKGLEWVSY |
| | | ISSGGSTIYYADSVKGRFTISRDNAKNSLYLQMNSLRAEDTAVYYCARDW |
| | | VDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPEAAGGPSVFLFPPKPKDTLMISRTPE |
| | | VTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVYPPSRDEL |
| | | TKNQVSLTCLVKGFYPSDIAVEWESNGQPENNYKTTPPVLDSDGSFALVSK |
| | | LTVDKSRWQQGNVFSCSVMHEALHNHYTQKSLSLSPGGGGGDIQMTQSPS |
| | | SLSASVGDRVTITCQASQSIYSSLAWYQQKPGKAPKLLIYDASHLASGVPSR |
| | | FSGSRYGTDFTLTISSVQPEDFATYYCQGGWYSSAATYVPNTFGGGTKVEV |
| | | KRTVAAPSVFIFPPSDEQLKSGTASVVCLLNNFYPREAKVQWKVDNALQSG |
| | | NSQESVTEQDSKDSTYSLSSTLTLSKADYEKVYACEVTHQGLSSPVTKS |
| | | FNRGEC |

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

1. An antibody construct comprising:
a) a first 4-1BB antigen-binding domain that binds to a 4-1BB extracellular domain (4-1BB ECD) and is derived from an agonistic anti-4-1BB antibody, and
b) a first tumor-associated antigen (TAA) antigen binding domain (TAA antigen-binding domain) that binds to a TAA,
wherein the first 4-1BB antigen-binding domain and the first TAA antigen-binding domain are linked directly or indirectly to a scaffold.

2.-4.(canceled)

5. The antibody construct according to claim 1, wherein the first 4-1BB antigen-binding domain comprises: a) a heavy chain variable domain comprising the three heavy chain CDRs of antibody 1C3, and a light chain variable domain comprising the three light chain CDRs of antibody 1C3; b) a heavy chain variable domain comprising the three heavy chain CDRs of antibody 1C8, and a light chain variable domain comprising the three light chain CDRs of antibody 1C8; c) a heavy chain variable domain comprising the three heavy chain CDRs of antibody 1G1, and a light chain variable domain comprising the three light chain CDRs of antibody 1G1; d) a heavy chain variable domain comprising the three heavy chain CDRs of antibody 2E8, and a light chain variable domain comprising the three light chain CDRs of antibody 2E8; e) a heavy chain variable domain comprising the three heavy chain CDRs of antibody 3E7, and a light chain variable domain comprising the three light chain CDRs of antibody 3E7; and f) a heavy chain variable domain comprising the three heavy chain CDRs of antibody 4E6, and a light chain variable domain comprising the three light chain CDRs of antibody 4E6; g) a heavy chain variable domain comprising the three heavy chain CDRs of antibody 5G8, and a light chain variable domain comprising the three light chain CDRs of antibody 5G8; or h) a heavy chain variable domain comprising the three heavy chain CDRs of antibody 6B3, and a light chain variable domain comprising the three light chain CDRs of antibody 6B3.

6. The antibody construct according to claim 5, wherein the first 4-1BB antigen-binding domain is a human or humanized antigen-binding domain.

7. The antibody construct according to claim 6, wherein the first 4-1BB antigen-binding domain comprises:
a) a heavy chain variable domain (VH) sequence that is at least 85% identical to the VH sequence of v28726 and a light chain variable domain (VL) sequence that is at least 85% identical to the VL sequence of v28726;
b) a heavy chain variable domain (VH) sequence that is at least 85% identical to the VH sequence of v28727 and a light chain variable domain (VL) sequence that is at least 85% identical to the VL sequence of v28727;
c) a heavy chain variable domain (VH) sequence that is at least 85% identical to the VH sequence of v28728 and a light chain variable domain (VL) sequence that is at least 85% identical to the VL sequence of v28728;
d) a heavy chain variable domain (VH) sequence that is at least 85% identical to the VH sequence of v28730 and a light chain variable domain (VL) sequence that is at least 85% identical to the VL sequence of v28730;
e) a heavy chain variable domain (VH) sequence that is at least 85% identical to the VH sequence of v28700 and a light chain variable domain (VL) sequence that is at least 85% identical to the VL sequence of v28700;
f) a heavy chain variable domain (VH) sequence that is at least 85% identical to the VH sequence of v28704 and a light chain variable domain (VL) sequence that is at least 85% identical to the VL sequence of v28704;
g) a heavy chain variable domain (VH) sequence that is at least 85% identical to the VH sequence of v28705 and a light chain variable domain (VL) sequence that is at least 85% identical to the VL sequence of v28705;
h) a heavy chain variable domain (VH) sequence that is at least 85% identical to the VH sequence of v28706 and a light chain variable domain (VL) sequence that is at least 85% identical to the VL sequence of v28706;
i) a heavy chain variable domain (VH) sequence that is at least 85% identical to the VH sequence of v28711 and a light chain variable domain (VL) sequence that is at least 85% identical to the VL sequence of v28711;
j) a heavy chain variable domain (VH) sequence that is at least 85% identical to the VH sequence of v28712 and a light chain variable domain (VL) sequence that is at least 85% identical to the VL sequence of v28712;
k) a heavy chain variable domain (VH) sequence that is at least 85% identical to the VH sequence of v28713 and a light chain variable domain (VL) sequence that is at least 85% identical to the VL sequence of v28713;
l) a heavy chain variable domain (VH) sequence that is at least 85% identical to the VH sequence of v28696 and a light chain variable domain (VL) sequence that is at least 85% identical to the VL sequence of v28696;
m) a heavy chain variable domain (VH) sequence that is at least 85% identical to the VH sequence of v28697 and a light chain variable domain (VL) sequence that is at least 85% identical to the VL sequence of v28697;
n) a heavy chain variable domain (VH) sequence that is at least 85% identical to the VH sequence of v28698 and a light chain variable domain (VL) sequence that is at least 85% identical to the VL sequence of v28698;
o) a heavy chain variable domain (VH) sequence that is at least 85% identical to the VH sequence of v28701 and a light chain variable domain (VL) sequence that is at least 85% identical to the VL sequence of v28701;
p) a heavy chain variable domain (VH) sequence that is at least 85% identical to the VH sequence of v28702 and a light chain variable domain (VL) sequence that is at least 85% identical to the VL sequence of v28702;
q) a heavy chain variable domain (VH) sequence that is at least 85% identical to the VH sequence of v28703 and a light chain variable domain (VL) sequence that is at least 85% identical to the VL sequence of v28703;
r) a heavy chain variable domain (VH) sequence that is at least 85% identical to the VH sequence of v28707 and a light chain variable domain (VL) sequence that is at least 85% identical to the VL sequence of v28707;
s) a heavy chain variable domain (VH) sequence that is at least 85% identical to the VH sequence of v28683 and a light chain variable domain (VL) sequence that is at least 85% identical to the VL sequence of v28683;
t) a heavy chain variable domain (VH) sequence that is at least 85% identical to the VH sequence of v28684 and a light chain variable domain (VL) sequence that is at least 85% identical to the VL sequence of v28684;
u) a heavy chain variable domain (VH) sequence that is at least 85% identical to the VH sequence of v28685 and a light chain variable domain (VL) sequence that is at least 85% identical to the VL sequence of v28685;
v) a heavy chain variable domain (VH) sequence that is at least 85% identical to the VH sequence of v28686 and a light chain variable domain (VL) sequence that is at least 85% identical to the VL sequence of v28686;
w) a heavy chain variable domain (VH) sequence that is at least 85% identical to the VH sequence of v28687 and a light chain variable domain (VL) sequence that is at least 85% identical to the VL sequence of v28687;
x) a heavy chain variable domain (VH) sequence that is at least 85% identical to the VH sequence of v28688 and a light chain variable domain (VL) sequence that is at least 85% identical to the VL sequence of v28688;
y) a heavy chain variable domain (VH) sequence that is at least 85% identical to the VH sequence of v28689 and a light chain variable domain (VL) sequence that is at least 85% identical to the VL sequence of v28689;
z) a heavy chain variable domain (VH) sequence that is at least 85% identical to the VH sequence of v28690 and a light chain variable domain (VL) sequence that is at least 85% identical to the VL sequence of v28690;
aa) a heavy chain variable domain (VH) sequence that is at least 85% identical to the VH sequence of v28691 and a light chain variable domain (VL) sequence that is at least 85% identical to the VL sequence of v28691;
bb) a heavy chain variable domain (VH) sequence that is at least 85% identical to the VH sequence of v28692 and a light chain variable domain (VL) sequence that is at least 85% identical to the VL sequence of v28692;
cc) a heavy chain variable domain (VH) sequence that is at least 85% identical to the VH sequence of v28694 and a light chain variable domain (VL) sequence that is at least 85% identical to the VL sequence of v28694;
dd) a heavy chain variable domain (VH) sequence that is at least 85% identical to the VH sequence of v28695 and a light chain variable domain (VL) sequence that is at least 85% identical to the VL sequence of v28695; or
ee) a heavy chain variable domain (VH) sequence that is at least 85% identical to the VH sequence of v28693 and a light chain variable domain (VL) sequence that is at least 85% identical to the VL sequence of v28693.

8. The antibody construct according to claim 7, further comprising a second 4-1BB antigen binding domain.

9. (canceled)

10. The antibody construct according to claim 8, wherein the second 4-1BB antigen-binding domain is the same as the first 4-1BB antigen-binding domain.

11. The antibody construct according to claim 10, wherein the first 4-1BB antigen-binding domain and/or the second 4-1BB antigen-binding domain are in a Fab format.

12. The antibody construct according to claim 11, wherein the first TAA antigen-binding domain is a folate receptor-α (FRα) antigen-binding domain, a Solute Carrier Family 34 Member 2 (NaPi2b) antigen-binding domain, a HER2 antigen-binding domain, a mesothelin antigen-binding domain, or a Solute Carrier Family 39 Member 6 (LIV-1) antigen-binding domain.

13. The antibody construct according to claim 12, wherein the antibody construct comprises a second TAA antigen-binding domain.

14. The antibody construct according to claim 13, wherein the first and second TAA antigen-binding domain bind to the same TAA.

15. The antibody construct according to claim 12, wherein the first TAA antigen-binding domain is a FRα antigen-binding domain.

16. The antibody construct according to claim 15, wherein the FRα antigen-binding domain comprises: a) a heavy chain variable domain comprising the three heavy chain CDRs of antibody 8K22, and a light chain variable domain comprising the three light chain CDRs of antibody 8K22; or b) heavy chain variable domain comprising the three heavy chain CDRs of antibody 1H06, and a light chain variable domain comprising the three light chain CDRs of antibody 1H06.

17. The antibody construct according to claim 16, wherein the FRα antigen-binding domain is a human or humanized antigen-binding domain.

18. The antibody construct according to claim 17, wherein the FRα antigen-binding domain comprises:
a) a heavy chain variable domain (VH) sequence that is at least 85% identical to the VH sequence of v23794 and a light chain variable domain (VL) sequence that is at least 85% identical to the VL sequence of v23794;
b) a heavy chain variable domain (VH) sequence that is at least 85% identical to the VH sequence of v23795 and a light chain variable domain (VL) sequence that is at least 85% identical to the VL sequence of v23795;
c) a heavy chain variable domain (VH) sequence that is at least 85% identical to the VH sequence of v23796 and a light chain variable domain (VL) sequence that is at least 85% identical to the VL sequence of v23796;
d) a heavy chain variable domain (VH) sequence that is at least 85% identical to the VH sequence of v23797 and a light chain variable domain (VL) sequence that is at least 85% identical to the VL sequence of v23797;
e) a heavy chain variable domain (VH) sequence that is at least 85% identical to the VH sequence of v23798 and a light chain variable domain (VL) sequence that is at least 85% identical to the VL sequence of v23798;
f) a heavy chain variable domain (VH) sequence that is at least 85% identical to the VH sequence of v23799 and a light chain variable domain (VL) sequence that is at least 85% identical to the VL sequence of v23799;
g) a heavy chain variable domain (VH) sequence that is at least 85% identical to the VH sequence of v23800 and a light chain variable domain (VL) sequence that is at least 85% identical to the VL sequence of v23800;
h) a heavy chain variable domain (VH) sequence that is at least 85% identical to the VH sequence of v23801 and a light chain variable domain (VL) sequence that is at least 85% identical to the VL sequence of v23801;
i) a heavy chain variable domain (VH) sequence that is at least 85% identical to the VH sequence of v23802 and a light chain variable domain (VL) sequence that is at least 85% identical to the VL sequence of v23802;
j) a heavy chain variable domain (VH) sequence that is at least 85% identical to the VH sequence of v23803 and a light chain variable domain (VL) sequence that is at least 85% identical to the VL sequence of v23803;
k) a heavy chain variable domain (VH) sequence that is at least 85% identical to the VH sequence of v23804 and a light chain variable domain (VL) sequence that is at least 85% identical to the VL sequence of v23804;
l) a heavy chain variable domain (VH) sequence that is at least 85% identical to the VH sequence of v23805 and a light chain variable domain (VL) sequence that is at least 85% identical to the VL sequence of v23805;
m) a heavy chain variable domain (VH) sequence that is at least 85% identical to the VH sequence of v23806 and a light chain variable domain (VL) sequence that is at least 85% identical to the VL sequence of v23806;
n) a heavy chain variable domain (VH) sequence that is at least 85% identical to the VH sequence of v23807 and a light chain variable domain (VL) sequence that is at least 85% identical to the VL sequence of v23807;
o) a heavy chain variable domain (VH) sequence that is at least 85% identical to the VH sequence of v23808 and a light chain variable domain (VL) sequence that is at least 85% identical to the VL sequence of v23808;
p) a heavy chain variable domain (VH) sequence that is at least 85% identical to the VH sequence of v23809 and a light chain variable domain (VL) sequence that is at least 85% identical to the VL sequence of v23809;
q) a heavy chain variable domain (VH) sequence that is at least 85% identical to the VH sequence of v23810 and a light chain variable domain (VL) sequence that is at least 85% identical to the VL sequence of v23810;
r) a heavy chain variable domain (VH) sequence that is at least 85% identical to the VH sequence of v23811 and a light chain variable domain (VL) sequence that is at least 85% identical to the VL sequence of v23811;
s) a heavy chain variable domain (VH) sequence that is at least 85% identical to the VH sequence of v23812 and a light chain variable domain (VL) sequence that is at least 85% identical to the VL sequence of v23812;
t) a heavy chain variable domain (VH) sequence that is at least 85% identical to the VH sequence of v23813 and a light chain variable domain (VL) sequence that is at least 85% identical to the VL sequence of v23813;
u) a heavy chain variable domain (VH) sequence that is at least 85% identical to the VH sequence of v23814 and a light chain variable domain (VL) sequence that is at least 85% identical to the VL sequence of v23814;
v) a heavy chain variable domain (VH) sequence that is at least 85% identical to the VH sequence of v23815 and a light chain variable domain (VL) sequence that is at least 85% identical to the VL sequence of v23815;
w) a heavy chain variable domain (VH) sequence that is at least 85% identical to the VH sequence of v23816 and a light chain variable domain (VL) sequence that is at least 85% identical to the VL sequence of v23816;
x) a heavy chain variable domain (VH) sequence that is at least 85% identical to the VH sequence of v23817 and a light chain variable domain (VL) sequence that is at least 85% identical to the VL sequence of v23817; or
y) a heavy chain variable domain (VH) sequence that is at least 85% identical to the VH sequence of v23818 and a light chain variable domain (VL) sequence that is at least 85% identical to the VL sequence of v23818.

19. The antibody construct according to claim 12, wherein the TAA antigen-binding domain is in an scFv format.

20. The antibody construct according to claim 12, wherein the TAA antigen-binding domain is in a Fab format.

21. The antibody construct according to claim 12, wherein the scaffold is a dimeric Fc construct having a first Fc polypeptide and a second Fc polypeptide, each Fc polypeptide comprising a CH3 sequence, or wherein the scaffold is a linker or an albumin polypeptide.

22. The antibody construct according to claim 21, wherein the scaffold is a heterodimeric Fc construct having a first Fc polypeptide that is different from the second Fc polypeptide, and wherein the CH3 sequences of the first Fc polypeptide and the second Fc polypeptide comprise amino acid substitutions that promote the formation of a heterodimeric Fc.

23. The antibody construct according to claim 22, wherein:
a) one Fc polypeptide comprises the amino acid substitutions T350V_L351Y_F405A_Y407V and the other Fc polypeptide comprises the amino acid substitutions T350V_T366L_K392L_T394W;
b) one Fc polypeptide comprises the amino acid substitutions T350V_T366L_K392M_T394W and the other Fc polypeptide comprises the amino acid substitutions T350V_L351Y_F405A_Y407V;
c) one Fc polypeptide comprises the amino acid substitutions L351Y_F405A_Y407V and the other Fc polypeptide comprises the amino acid substitutions T366L_K392M_T394W;
d) one Fc polypeptide comprises the amino acid substitutions L351Y_F405A_Y407V and the other Fc polypeptide comprises the amino acid substitutions T366L_K392L_T394W; or e) one Fc polypeptide comprises the amino acid substitutions L351Y_S400E_F405A_Y407V and the other Fc polypeptide comprises the amino acid substitutions T366I_N390R_K392M_T394W,
wherein the numbering of residues is according to the EU numbering system.

24. The antibody construct according to claim 21, further comprising one or more amino acid modifications that reduce effector function.

25. (canceled)

26. The antibody construct according to claim 1, wherein the first 4-1BB antigen-binding domain is linked to the N terminus of the first Fc polypeptide, and the first TAA antigen-binding domain is linked to the C terminus of the first Fc polypeptide.

27. The antibody construct according to claim 1, wherein the first 4-1BB antigen-binding domain is linked to the N terminus of the first Fc polypeptide, and the first TAA antigen-binding domain is linked to the C terminus of the second Fc polypeptide

28. The antibody construct according to claim 8, wherein the first 4-1BB antigen-binding domain is linked to the N terminus of the first Fc polypeptide, and the first TAA antigen-binding domain is linked to the C terminus of the first Fc polypeptide, and the second 4-1BB antigen-binding domain is linked to the N terminus of the second Fc polypeptide.

29. (canceled)

30. The antibody construct according to claim 13, wherein the first 4-1BB antigen-binding domain is linked to the N terminus of the first Fc polypeptide or to the N terminus of the second Fc polypeptide, the first TAA antigen-binding domain is linked to the C terminus of the first Fc polypeptide and a second TAA antigen-binding domain is linked to the C terminus of the second Fc polypeptide.

31. The antibody construct according to claim 15, wherein the first and second 4-1BB antigen-binding domains comprise a heavy chain variable domain comprising the three heavy chain CDRs of antibody 1G1, and a light chain variable domain comprising the three light chain CDRs of antibody 1G1, and the first and/or second FRα antigen-binding domain comprises a heavy chain variable domain comprising the three heavy chain CDRs of antibody 8K22, and a light chain variable domain comprising the three light chain CDRs of antibody 8K22.

32. The antibody construct according to claim 31, wherein the first and second 4-1BB antigen-binding domain comprises a heavy chain variable domain (VH) sequence that is at least 85% identical to the VH sequence of v28684 and a light chain variable domain (VL) sequence that is at least 85% identical to the VL sequence of v28684, and the first and/or second FRα antigen-binding domain comprises a heavy chain variable domain (VH) sequence that is at least 85% identical to the VH sequence of v23807 and a light chain variable domain (VL) sequence that is at least 85% identical to the VL sequence of v23807.

33. The antibody construct according to claim 32, comprising a first heavy chain polypeptide sequence as set forth in SEQ ID NO:353, a second heavy chain polypeptide sequence as set forth in SEQ ID NO:349, and a light chain polypeptide sequence as set forth in SEQ ID NO:346.

34. The antibody construct according to claim 1, conjugated to a drug.

35. A pharmaceutical composition comprising the antibody construct of claim 1.

36. One or more nucleic acids encoding the antibody construct according to claim 1.

37. One or more vectors comprising the one or more nucleic acids according to claim 36.

38. An isolated cell comprising the one or more nucleic acid according to claim 36.

39. A method of preparing the antibody construct according to claim 1, comprising culturing an isolated cell of claim 38 comprising one or more nucleic acids encoding the antibody construct under conditions suitable for expressing the antibody construct, and purifying the antibody construct.

40. A method of treating a subject having a cancer, comprising administering to the subject an effective amount of the antibody construct according to claim 1.

41.-43. (canceled)

44. An antibody construct or antigen-binding fragment thereof, that specifically binds to 4-1BB, comprising: a heavy chain variable sequence comprising three heavy chain CDRs and a light chain variable sequence comprising three light chain CDRs, wherein the heavy chain CDRs and the light chain CDRs are from any one of antibodies 1G1, 1B2, 1C3, 1C8, 2A7, 2E8, 2H9, 3D7, 3H1, 3E7, 3G4, 4B11, 4E6, 4F9, 4G10, 5E2, 5G8, or 6B3.

45.-64. (canceled)

65. An antibody construct or antigen-binding fragment thereof, that specifically binds to FRα, comprising: a heavy chain variable (VH) sequence comprising three CDRs and a light chain variable (VL) sequence comprising three CDRs, wherein the heavy chain CDRs and the light chain CDRs are from antibody 8K22 or 1H06.

66.-83. (canceled)
